SEQUENCE LISTING (18 sequences)

SEQ ID NO: 1 - protein, 403 aa, Fusarium oxysporum
  P450 Nor, crystal structure 1rom
MASGAPSFPFSRASGPEPPAEFAKLRATNPVSQVKLFDGSLAWLVTKHKDVCFVATSEKLSKVRTRQGFPELSASGKQAAKAKPTFVDMDPPEHMHQRSMVEPTFTPEAVKNLQPYIQRTVDDLLEQMKQKGCANGPVDLVKEFALPVPSYIIYTLLGVPFNDLEYLTQQNAIRTNGSSTAREASAANQELLDYLAILVEQRLVEPKDDIISKLCTEQVKPGNIDKSDAVQIAFLLLVAGNATMVNMIALGVATLAQHPDQLAQLKANPSLAPQFVEELCRYHTASALAIKRTAKEDVMIGDKLVRANEGIIASNQSANRDEEVFENPDEFNMNRKWPPQDPLGFGFGDHRCIAEHLAKAELTTVFSTLYQKFPDLKVAVPLGKINYTPLNRDVGIVDLPVIF

SEQ ID NO: 2 - protein, 403 aa, Saccharopolyspora erythraea
  P450 EryF, crystal structure 1oxa
ATVPDLESDSFHVDWYSTYAELRETAPVTPVRFLGQDAWLVTGYDEAKAALSDLRLSSDPKKKYPGVEVEFPAYLGFPEDVRNYFATNMGTSDPPTHTRLRKLVSQEFTVRRVEAMRPRVEQITAELLDEVGDSGVVDIVDRFAHPLPIKVICELLGVDEAARGAFGRWSSEILVMDPERAEQRGQAAREVVNFILDLVERRRTEPGDDLLSALISVQDDDDGRLSADELTSIALVLLLAGFEASVSLIGIGTYLLLTHPDQLALVRADPSALPNAVEEILRYIAPPETTTRFAAEEVEIGGVAIPQYSTVLVANGAANRDPSQFPDPHRFDVTRDTRGHLSFGQGIHFCMGRPLAKLEGEVALRALFGRFPALSLGIDADDVVWRRSLLLRGIDHLPVRLDG

SEQ ID NO: 3 - protein, 412 aa, Pseudomonas sp.
  P450 Terp, crystal structure 1cpt
MDARATIPEHIARTVILPQGYADDEVIYPAFKWLRDEQPLAMAHIEGYDPMWIATKHADVMQIGKQPGLFSNAEGSEILYDQNNEAFMRSISGGCPHVIDSLTSMDPPTHTAYRGLTLNWFQPASIRKLEENIRRIAQASVQRLLDFDGECDFMTDCALYYPLHVVMTALGVPEDDEPLMLKLTQDFFGVEAARRFHETIATFYDYFNGFTVDRRSCPKDDVMSLLANSKLDGNYIDDKYINAYYVAIATAGHDTTSSSSGGAIIGLSRNPEQLALAKSDPALIPRLVDEAVRWTAPVKSFMRTALADTEVRGQNIKRGDRIMLSYPSANRDEEVFSNPDEFDITRFPNRHLGFGWGAHMCLGQHLAKLEMKIFFEELLPKLKSVELSGPPRLVATNFVGGPKNVPIRFTKA

SEQ ID NO: 4 - protein, 414 aa, Pseudomonas putida
  P450 Cam, crystal structure 3cpp
TTETIQSNANLAPLPPHVPEHLVFDFDMYNPSNLSAGVQEAWAVLQESNVPDLVWTRCNGGHWIATRGQLIREAYEDYRHFSSECPFIPREAGEAYDFIPTSMDPPEQRQFRALANQVVGMPVVDKLENRIQELACSLIESLRPQGQCNFTEDYAEPFPIRIFMLLAGLPEEDIPHLKYLTDQMTRPDGSMTFAEAKEALYDYLIPIIEQRRQKPGTDAISIVANGQVNGRPITSDEAKRMCGLLLVGGLDTVVNFLSFSMEFLAKSPEHRQELIERPERIPAACEELLRRFSLVADGRILTSDYEFHGVQLKKGDQILLPQMLSGLDERENACPMHVDFSRQKVSHTTFGHGSHLCLGQHLARREIIVTLKEWLTRIPDFSIAPGAQIQHKSGIVSGVQALPLVWDPATTKAV

SEQ ID NO: 5 - protein, 471 aa, Bacillus megaterium
  P450 BM3, crystal structure 2hpd
TIKEMPQPKTFGELKNLPLLNTDKPVQALMKIADELGEIFKFEAPGRVTRYLSSQRLIKEACDESRFDKNLSQALKFVRDFAGDGLFTSWTHEKNWKKAHNILLPSFSQQAMKGYHAMMVDIAVQLVQKWERLNADEHIEVPEDMTRLTLDTIGLCGFNYRFNSFYRDQPHPFITSMVRALDEAMNKLQRANPDDPAYDENKRQFQEDIKVMNDLVDKIIADRKASGEQSDDLLTHMLNGKDPETGEPLDDENIRYQIITFLIAGHETTSGLLSFALYFLVKNPHVLQKAAEEAARVLVDPVPSYKQVKQLKYVGMVLNEALRLWPTAPAFSLYAKEDTVLGGEYPLEKGDELMVLIPQLHRDKTIWGDDVEEFRPERFENPSAIPQHAFKPFGNGQRACIGQQFALHEATLVLGMMLKHFDFEDHTNYELDIKETLTLKPEGFVVKAKSKKIPLGGIPSPSTEQSAKKVR

SEQ ID NO: 6 - protein, 473 aa, Rabbit
  P450 2C5, crystal structure 1dt6
MAKKTSSKGKLPPGPTPFPIIGNILQIDAKDISKSLTKFSECYGPVFTVYLGMKPTVVLHGYEAVKEALVDLGEEFAGRGSVPILEKVSKGLGIAFSNAKTWKEMRRFSLMTLRNFGMGKRSIEDRIQEEARCLVEELRKTNASPCDPTFILGCAPCNVICSVIFHNRFDYKDEEFLKLMESLHENVELLGTPWLQVYNNFPALLDYFPGIHKTLLKNADYIKNFIMEKVKEHQKLLDVNNPRDFIDCFLIKMEQENNLEFTLESLVIAVSDLFGAGTETTSTTLRYSLLLLLKHPEVAARVQEEIERVIGRHRSPCMQDRSRMPYTDAVIHEIQRFIDLLPTNLPHAVTRDVRFRNYFIPKGTDIITSLTSVLHDEKAFPNPKVFDPGHFLDESGNFKKSDYFMPFSAGKRMCVGEGLARMELFLFLTSILQNFKLQSLVEPKDLDITAVVNGFVSVPPSYQLCFIPIHHHH

SEQ ID NO: 7 - protein, 487 aa, Rabbit
  P450 2C5
MDPVVVLVLGLCCLLLLSIWKQNSGRGKLPPGPTPFPIIGNILQIDAKDISKSLTKFSECYGPVFTVYLGMKPTVVLHGYEAVKEALVDLGEEFAGTGSVPILEKVSKGLGIAFSNAKTWKEMRRFSLMTLRNFGMGKRSIEDRIQEEARCLVEELRKTNASPCDPTFILGCAPCNVICSVIFHNRFDYKDEEFLKLMESLNENVRILSSPWLQVYNNFPALLDYFPGIHKTLLKNADYIKNFIMEKVKEHEKLLDVNNPRDFIDCFLIKMEQENNLEFTLESLVIAVSDLFGAGTETTSTTLRYSLLLLLKHPEVAARVQEEIERVIGRHRSPCMQDRSRMPYTDAVIHEIQRFIDLLPTNLPHAVTRDVRFRNYFIPKGTDIITSLTSVLHDEKAFPNPKVFDPGHFLDESGNFKKSDYFMPFSAGKRMCVGEGLARMELFLFLTSILQNFKLQSLVEPKDLDITAVVNGFVSVPPSYQLCFIPI

SEQ ID NO: 8 - protein, 455 aa, Mycobacterium tuberculosis
  CYP51, crystal structure 1e9x
MSAVALPRVSGGHDEHGHLEEFRTDPIGLMQRVRDECGDVGTFQLAGKQVVLLSGSHANEFFFRAGDDDLDQAKAYPFMTPIFGEGVVFDASPERRKEMLHNAALRGEQMKGHAATIEDQVRRMIADWGEAGEIDLLDFFAELTIYTSSACLIGKKFRDQLDGRFAKLYHELERGTDPLAYVDPYLPIESFRRRDEARNGLVALVADIMNGRIANPPTDKSDRDMLDVLIAVKAETGTPRFSADEITGMFISMMFAGHHTSSGTASWTLIELMRHRDAYAAVIDELDELYGDGRSVSFHALRQIPQLENVLKETLRLHPPLIILMRVAKGEFEVQGHRIHEGDLVAASPAISNRIPEDFPDPHDFVPARYEQPRQEDLLNRWTWIPFGAGRHRCVGAAFAIMQIKAIFSVLLREYEFEMAQPPESYRNDHSKMVVQLAQPACVRYRRRTGVHHHH

SEQ ID NO: 9 - protein, 504 aa, Rat
  CYP3A1
MDLLSALTLETWVLLAVVLVLLYGFGTRTHGLFKKQGIPGPKPLPFFGTVLNYYMGLWKFDVECHKKYGKIWGLFDGQMPLFAITDTEMIKNVLVKECFSVFTNRRDFGPVGIMGKAVSVAKDEEWKRYRALLSPTFTSGRLKEMFPIIEQYGDILVKYLKQEAETGKPVTMKKVFGAYSMDVITSTSFGVNVDSLNNPKDPFVEKTKKLLRFDFFDPLFLSVVLFPFLTPIYEMLNICMFPKDSIEFFKKFVYRMKETRLDSVQKHRVDFLQLMMNAHNDSKDKESHTALSDMEITAQSIIFIFAGYEPTSSTLSFVLHSLATHPDTQKKLQEEIDRALPNKAPPTYDTVMEMEYLDMVLNETLRLYPIGNRLERVCKKDVEINGVFMPKGSVVMIPSYALHRDPQHWPEPEEFRPERFSKENKGSIDPYVYLPFGNGPRNCIGMRFALMNMKLALTKVLQNFSFQPCKETQIPLKLSRQGLLQPTKPIILKVVPRDEIITGS

SEQ ID NO: 10 - protein, 503 aa, Homo sapiens
  CYP3A3
ALIPDLAMETWLLLAVSLVLLYLYGTHSHGLFKKLGIPGPTPLPFLGNILSYHKGFCMFDMECHKKYGKVWGFYDGQQPVLAITDPDMIKLVLVKECYSVFTNREPFGPVGFMKSAISIAEDEEWKRLRSLLSPTFTSGKLKEMVPIIAQYGDVLVRNLRRERETGKPVTLKDVFGAYSMDVITSSSFGVNVDSLNNPQDPLVENTKKLLRFDFLDPFFLSITVFPFLIPILEVLNICVFPREVTNFLRKAVKRMKESRLEDTQKHRVDFLQLMIDSHKNSKETESHKALSDLELVAQSIIFIFAGYETTSSVLSFIMYELATHPDVQQKLQEEIDAVLPNKAPPTYDTVLQMEYLDMVVNETLRLFPIAMRLERVCKKDVEINGMFIPKGWVVMIPSYALHRDPKYWTEPEKFLPERFSKKNKDNIDPYIYTPFGSGPRNCIGMRFALMNMKLALIRVLQNFSFKPCKETQIPLKLSLGGLLQPEKPVVLKVESRDGTVSGA

SEQ ID NO: 11 - protein, 502 aa, Homo sapiens
  CYP3A4
ALIPDLAMETWLLLAVSLVLLYLYGTHSHGLFKKLGIPGPTPLPFLGNILSYHKGFCMFDMECHKKYGKVWGFYDGQQPVLAITDPDMIKTVLVKECYSVFTNRRPFGPVGFMKSAISIAEDEEWKRLRSLLSPTFTSGKLKEMVPIIAQYGDVLVRNLRREAETGKPVTLKDVFGAYSMDVITSTSFGVNIDSLNNPQDPFVENTKKLLRFDFLDPFFLSITVFPFLIPILEVLNICVFPREVTNFLRKSVKRMKESRLEDTQKHRVDFLQLMIDSQNSKETESHKALSDLELVAQSIIFIFAGYETTSSVLSFIMYELATHPVDQQKLQEEIDAVLPNKAPPTYDTVLQMEYLDMVVNETLRLFPIAMRLERVCKKDVEINGMFIPKGWVVMIPSYALHRDPKYMTEPEKFLPERFSKKNKDNIDPYIYTPFGSGPRNCIGMRFALMNMKLALIRVLQNFSFKPCKETQIPLKLSLGGLLQPEKPVVLKVESRDGTVSGA

SEQ ID NO: 12 - protein, 502 aa, Homo sapiens
  CYP3A5
MDLIPNLAVETWLLLAVSLVLLYLYGTRTHGLFKRLGIPGPTPLPLLGNVLSYRQGLWKFDTECYKKYGKMWGTYEGQLPVLAITDPDVIRTVLVKECYSVFTNRRSLGPVGFMKSAISLAEDEEWKRIRSLLSPTFTSGKLKEMFPIIAQYGDVLVRNLRREAEKGKPVTLKDIFGAYSMDVITGTSFGVNIDSLNNPQDPFVESTKKFLKFGFLDPLFLSIILFPFLTPVFEALNVSLFPKDTINFLSKSVNRMKKSRLNDKQKHRLDFLQLMIDSQNSKETESHKALSDLELAAQSIIFIFAGYETTSSVLSFTLYELATHPDVQQKLQKEIDAVLPNKAPPTYDAVVQMEYLDMVVNETLRLFPVAIRLERTCKKDVEINGVFIPKGSMVVIPTYALHHDPKYWTEPEEFRPERFSKKKDSIDPYIYTPFGTGPRNCIGMRFALMNMKLALIRVLQNFSFKPCKETQIPLKLDTQGLLQPEKPIVLKVDSRDGTLSGE

SEQ ID NO: 13 - protein, 503 aa, Homo sapiens
  CYP3A43
MDLIPNFAMETWVLVATSLVLLYIYGTHSHKLFKKLGIPGPTPLPFLGTILFYLRGLWNFDRECNEKYGEMWGLYEGQQPMLVIMDPDMIKTVLVKECYSVFTNQMPLGPMGFLKSALSFAEDEEWKRIRTLLSPAFTSVKFKEMVPIISQCGDMLVRSLRQEAENSKSINLKDFFGAYTMDVITGTLFGVNLDSLNNPQDPFLKNMKKLLKLDFLDPFLLLISLFPFLTPVFEALNIGLFPKDVTHFLKNSIERMKESRLKDKQKHRVDFFQQMIDSQNSKETKSHKALSDLELVAQSIIIIFAAYDTTSTTLPFIMYELATHPDVQQKLQEEIDAVLPNKAPVTYDALVQMEYLDMVVNETLRLFPVVSRVTRVCKKDIEINGVFIPKGLAVMVPIYALHHDPKYWTEPEKFCPERFSKKNKDSIDLYRYIPFGAGPRNCIGMRFALTNIKLAVIRALQNFSFKPCKETQIPLKLDNLPILQPEKPIVLKVHLRDGITSGP

SEQ ID NO: 14 - protein, 501 aa, Rabbit
  CYP3A6
MDLIFSLETWVLLAASLVLLYLYGTSTHGLFKKMGIPGPTPLPFIGTILEYRKGIWDFDIECRKKYGKMWGLFDGRQPLMVITDPDMIKTVLVKECYSVFTNRRSFGPVGFMKKAVSISEDEDWKRVRTLLSPTFTSGKLKEMLPIIAQYGDVLVKNLRQEAEKGKPVDLKEIFGAYSMDVITGTSFGVNIDSLRNPQDPFVKNVRRLLKFSFFDPLLLSITLFPFLTPIFEALHISMFPKDVMDFLKTSVEKIKDDRLKDKQKRRVDFLQLMINSQNSKEIDSHKALDDIEVVAQSIIILFAGYETTSSTLSFIMHLLATHPDVQQKLQEEIDTLLPNKELATYDTLVKMEYLDMVVNETLRLYPIAGRLERVCKKDVDINGTFIPKGTIVMMPTYALHRDPQHWTEPDEFRPERFSKKNKDNINPYIYHPFGAGPRNCLGMRFALMNIKLALVRLMQNFSFKLCKETQVPLKLGKQGLLQPEKPIVLKVVSRDGIIRGA

SEQ ID NO: 15 - protein, 503 aa, Homo sapiens
  CYP3A7
MDLIPNLAVETWLLLAVSLILLYLYGTRTHGLFKKLGIPGPTPLPFLGNALSFRKGYWTFDMECYKKYRKVWGIYDCQQPMLAITDPDMIKTVLVKECYSVFTNRRPFGPVGFMKNAISIAEDEEWKRIRSLLSPTFTSGKLKEMVPIIAQYGDVLVRNLRREAETGKPVTLKHVFGAYSMDVITSTSFGVSIDSLNNPQDPFVENTKKLLRFNPLDPFVLSIKVFPFLTPILEALNITVFPRKVISFLTKSVKQIKEGRLKETQKHRVDFLQLMIDSQNSKDSETHKALSDLELMAQSIIFIFAGYETTSSVLSFIIYELATHPDVQQKVQKEIDTVLPNKAPPTYDTVLQLEYLDMVVNETLRLFPVAMRLERVCKKDVEINGMFIPKGVVVMIPSYVLHHDPKYWTEPEKFLPERFSKKNKDNIDPYIYTPFGSGPRNCIGMRFALVNMKLALVRVLQNFSFKPCKETQIPLKLRFGGLLLTEKPIVLKAESRDETVSGA

SEQ ID NO: 16 - protein, 503 aa, Dog
  CYP3A12
MDLIPSFSTETWLLLAISLVLLYLYGTYTHGIFRKLGIPGPTPLPFVGTALGYRNGFYVFDMKCFSKYGRMWGFYDGRQPVLAITDPDMIKTVLVKECYSVFTNRRTLGPVGFMKSAISLSEDEEWKRMRTLLSPTFTTGKLKEMFPIIGQYGDVLVNNLRKEAEKGKAINLKDVFGAYSMDVITSTSFGVNIDSLNHPQDPFVENTKKLLKFDFLDPFFFSILLFPFLTPVFEILNIWLFPKKVTDFFRKSVERMKESRLKDKQKHRVDFLQLMINSQNSKEMDTHKALSDLELVAQSIIFIFAGYETTSTSLSFLMYELATHPDVQQKLQEEIDATFPNKALPTYDALVQMEYLDMVLNETLRLYPIAGRLERVCKKDVEISGVFIPKGTVVMVPTFTLHRDQSLWPEPEEFRPERFSRKNKDSINPYTYLPFGTGPRNCIGMRFAIMNMKLALVRVLQNFSFKPCKETQIPLKLNAQGIIQPEKPIVLKVEPRDGSVNGA

SEQ ID NO: 17 - protein, 503 aa, Pig
  CYP3A29
MDLIPGFSTETWVLLATSLVLLYLYGTYSHGLFKKLGIPGPRPLPYFGNILGYRKGVDHFDKKCFQQYGKMWGVYDGRQPLLAVTDPNMIKSVLVKECYSVFTNRRSFGPLGAMRNALSLAEDEEWKRIRTLLSPTFTSGKLKEMFPIISHYGDLLVSNLRKEAEKGKPVTMKDIFGAYSMDVITSTAFGVNIDSLNNPQDPFVENSKKLLKFSFFDPFLLSLIFFPFLTPIFEVLNITLFPKSSVNFFTKSVKRMKESRLTDQQKRRVDLLQLMINSQNSKEMDPHKSLSNEELVAQGIIFIFAGYETTSSALSLLAYELATHPDVQQKLQEEIEATFPNKAPPTYDALAQMEYLDMVVNETLRLYPIAARLERACKKDVEIHGVFVPKGTVVVVPVFVLHRDPDLWPEPEEFRPERFSKKHKDTINPYTYLPFGTGPRNCIGMRFALMNMKLALVRVLQNFSFKPCKETQIPLKLTTQGLTQPEKPVVLKILPRDGTVSGA

SEQ ID NO: 18 - protein, 503 aa, Mouse
  CYP3A13
MDLIPNFSMETWMLLATSLVLLYLYGTHSHGIFKKLGIPGPKPLPFLGTILAYQKGFWECDIQCHKKYGKMWGLYDGRQPVLAITDPDIIKTVLVKECYSTFTNRRRFGPVGILKKAISISENEEWKRIRALLSPTFTSGRLKEMFPIINQFTDVLVRNMRQGLGEGKPTSMKDIFGAYSMDVITATSFGVNIDSLNNPQDPFVEKIKKLLKFDIFDPLFLSVTLFPFLTPVFDALNVSLFPRDVISFFTTSVERMKENRMKEKEKQRVDFLQLMINSQNYKTKESHKALSDVEIVAQSVIFIFAGYETTSSALSFALYLLAIHPDVQKKLQDEIDAALPNKAPATYDTLLQMEYLDMVVNETLRLYPIAGRLERVCKTDVEINGLFIPKGTVVMIPTFALHKDPKYWPEPEEFRPERFSKKNQDSINPYMYLPFGSGPRNCIGMRFALINMKVALVRVLQNFTVQPCKETEIPLKLSKQGLLQPENPLLLKVVSRDETVSDE

(19) United States
(12) Patent Application Publication
(10) Pub. No.: US 2006/0099667 A1
(43) Pub. Date: May 11, 2006

(54) Method for performing restrained dynamics docking of one or multiple substrates on multi-specific enzymes

(76) Inventors: Francois Andre, Montigny-Le-Bretonneux (FR); Marcel Delaforge, Evry (FR); Nicolas Loiseau, Colomiers (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 10/532,868

(22) PCT Filed: Oct. 28, 2003
(86) PCT No.: PCT/IB03/05134

§ 371 Date: Apr. 28, 2005

Related U.S. Application Data
(60) Provisional application No. 60/421,569, filed on Oct. 28, 2002.

(51) Int. Cl. G06F 19/00; C12Q 1/26; C12N 9/02

(52) U.S. Cl. 435/25; 435/189; 702/19

(57) ABSTRACT

The present invention relates to a method for performing restrained dynamics docking of one or several substrates having allosteric or synergistic effect on enzymes presenting multipspecific and flexible active site. It also concerns a method for determining the 3D-substrates, which is the case for multispecific enzymes such as cytochrome P450, and specifically to cytochrome P450 3A4 and P450 3A7.

The present invention relates to a method for performing restrained dynamics docking of one or several substrates having allosteric or synergistic effect on enzymes presenting multispecific and flexible active site. It also concerns a method for determining the 3D-structure of active sites that are flexible and can adapt to different substrates, which is the case for multispecific enzymes such as cytochrome P450.

As of today, various computer graphics systems allow to generate molecular models of large molecules such as proteins from the PDB structural data obtained using X-ray crystallography and NMR. We can cite for example MODELLER, COMPOSER, MATCHMAKER (Tripos), or 3D graphical environments for molecular modeling such as SYBYL (Tripos) or INSIGHT II (Accelrys).

Substrates as well as inhibitors or agonists often act by binding to particular regions of an enzyme or receptor referred as the active site. In industry, the purpose of using these 3D models is to assess the main features of the molecules which are involved in the binding to the active site. New molecules that fit the active site can be designed.

Biological interactions are not possible without flexibility and motion. One of the principal tools in the theoretical study of motion in biological molecules is the method of molecular dynamics simulations (MD). This computational method calculates the time dependent behavior of a molecular system (Karplus and McCammon, 2002). MD simulations have provided detailed information on the fluctuations and conformational changes of proteins and nucleic acids. These methods are now routinely used to investigate the structure, dynamics and thermodynamics of biological molecules and their complexes. They are also used in the determination of structures from x-ray crystallography and from NMR experiments. The molecular dynamics simulations can be used to recreate the successive events in the binding process of a molecule, and thermodynamic parameters implicated in such process can therefore be derived, which is of great interest in the design of active molecules.

Nevertheless, the methods proposed in the art are based on a relatively low level of calculations of few parameters. It relies only on the molecule energy constrained with a fixed geometry. It relies only on the interaction energy between the molecule and the active site frozen in a fixed geometry.

Consequently, there is a need for a model replicating in silico the natural process of molecular interactions.

The method according to the invention provides both minimizations and molecular dynamics calculations. More specifically, it provides a new approach which is more appropriate to flexible structures, hereafter referred as “restrained dynamics docking” or “soft-restrained restrained dynamics docking”. This technique employs constrained dynamics simulations, where the only constraints are active site-substrate distances.

For example, to explain and predict drug metabolism in organisms, in which the cytochrome P450 (CYP) superfamily of haem-thiolate enzymes plays a central role, it is of large interest to dispose of a molecular picture of the binding sites responsible for the biotransformation. Efficiency of the prediction is then directly related to the molecular precision of the model, which resolution must be obtained at the atomic level to exploit the model for further docking studies.

In mammalian, hepatic cytochrome P450s constitute the major enzymes involved in the metabolism of exogenic compounds. Among them, isozymes of the CYP3 family (such as CYP3A1 and 3A2 in rat, and CYP3A4, CYP3A5, CYP3A7, CYP 3A43 in human) are known to metabolize the majority of drugs in clinical use. These are multi-specific enzymes, able to metabolize a large variety of structurally diverse chemicals or substrates including steroids, linear or cyclized peptides (Delaforge et al. 1997, Delaforge et al. 2001, Aninat et al. 2001), generally fairly lipophilic, within a broad range of molecular sizes from testosterone (Mw 288) to cyclosporin A (Mw 1203).

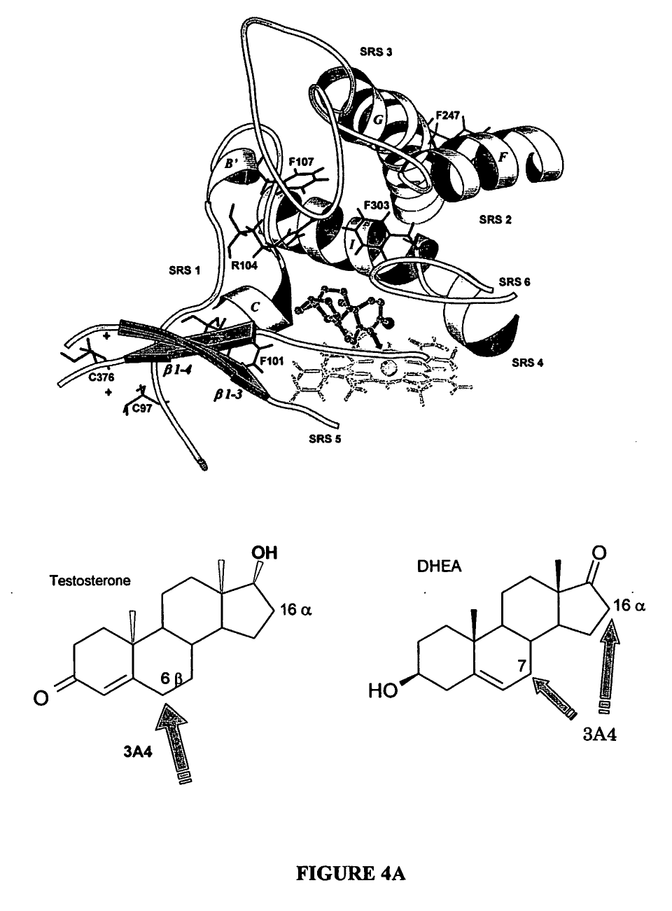

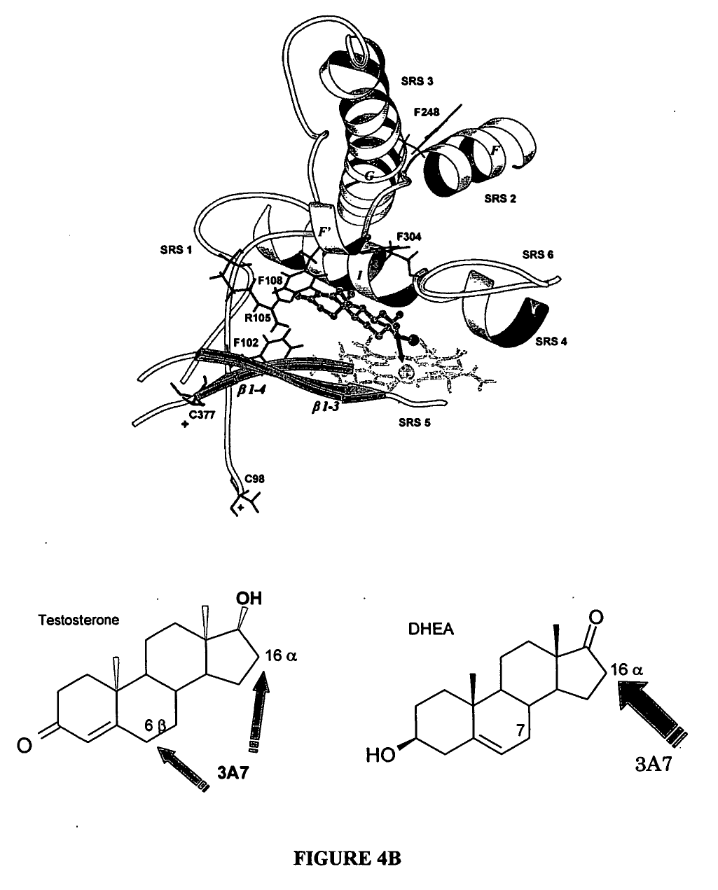

The inventory of known substrates for CYP 3A contains a large variety of different molecules having apparently no common structural factors. Actually it can be estimated that more than five hundred utilized drugs can be recognized and metabolized by CYP 3A (Guengerich 1995, Wrighton et al. 2000, Lewis 2001). Closer inspection of the precise transformations catalyzed by CYP 3A indicates that there is an important regio- and stereo-selectivity for each substrate. The active site can accommodate relatively rigid substrates such as aflatoxin derivatives or steroids, that are oxidized almost exclusively at a precise position. Thus CYP 3A4 catalyzes the testosterone oxidation exclusively at the 6β position, whereas CYP 3A7 oxidizes dehydroepiandrosterone (DHEA) or its 3 sulfate conjugate exclusively on the 16α position (see FIGS. 4A and 4B). In addition to such small substrates, CYP 3A metabolize also large molecules such as cyclosporin A (MW 1202), macrolide antibiotics (MW around 600) or ergot derivatives (MW from 500 to 700).

The recognized substrates can have endogenous origin such as steroids or can be drugs or compounds found in food. For example, grapefruit juice contains bergamottin derivatives having specific CYP 3A inhibitory activities (Schmiedlin-Ren et al. 1997). Linear peptides (Delaforge et al. 2001, Hosea et al. 2000) or cyclized peptides (Delaforge et al. 1997) containing from 2 aminoacids (called diketopiperazine, Delaforge et al. 2001, Aninat et al. 2001) to 11 amino-acids (e.g. cyclosporin) are also recognized.

Following this wide range substrate recognition, a tentative subclassification was established leading to a multi-site hypothesis (Hosea et al. 2000, Ekins et al. 2003) consisting of at least 2 or 3 binding zones in the active site. This hypothesis has been established on the facts that CYP 3A shows often atypical hyperbolic kinetic constants and is thus unable to reach saturation. In addition, the presence in the active site of a second substrate having a different molecular nature lead to either no modification or increased metabolism of both substrates. Such allosteric effects have been clearly described in the case of simultaneous metabolism of steroids such as testosterone and α-natphtoflavone.

Consequently, any molecular model describing correctly the multiple substrate specificity (that takes into account large variations in molecular size and chemical structures), and substrate cooperativity effects within the active site (when two or more drugs interact), is of considerable scientific and industrial interest. Such a molecular model must be able to rationalize the binding of the diverse known substrates, and the orientations of the molecules in the binding site that account for their known positions of metabolism (such as N-demethylations, benzylic hydroxylations etc.). CYP3A4 is considered as the main hepatic form and is found in a wide variety of human organs such as intestine, brain or skin. CYP 3A5 is also present in liver and is the major 3A form present in the kidney. The 3A5 isoform is subject to genetic polymorphism. CYP 3A7 is the major 3A isoform present in the foetus whereas CYP3A43 is mainly located in adult prostate or testis. These isoforms share amino acid identities higher than 70%. (Westlind-Johnsson et al. 2003, Gellner et al. 2001, Koch et al. 2002). It is currently accepted that CYP3A4 is the most active isoform for classical P450 3A substrates whereas recent data (Williams et al. 2002) demonstrate equal or slightly reduced activity for CYP3A5 and a significantly lower metabolism capability for CYP3A7 as compared to CYP3A4. Additionally, differences have been observed in term of oxidative regioselectivity of the CYP3A7 compared to other isoforms. As an example, CYP3A7 metabolizes intensively DHEA and especially its sulfate conjugate derivative whereas CYP3A4 is a poor metabolizer. The oxidation by CYP3A7 occurs mostly in the 16α position of DHEA. In contrast, CYP3A7 metabolizes testosterone in both 6β and 16α position whereas CYP3A4 or 3A5 metabolize it almost exclusively in the 6β position (Inoue et al. 2000).

At the contrary of the P450 3A subfamily, other P450 isoforms have more rigid active site, as suggested by the narrow range of recognized substrates or inhibitors. These P450 isoforms recognize generally a small number of substrates or inhibitors having in common the same shape (i.e. P450 1A isoforms), or the same charge (i.e. CYP 2B, 2C or 2D isoforms), or the same chemical nature such as steroids (i.e. CYP19 or CYP21 isoforms) or lipids (i.e. CYP 4 family).

As no high-resolution 3D structure of CYP3A is today publicly available, due to continuing difficulties in promoting crystallization of intrinsic membrane proteins or due to an unusual confornational flexibility that would explain how CYP3A can accommodate various substrates, it is necessary to rebuild a 3D model structure, integrating the known biochemical data of CYP3A and the structural data of other members of the CYP superfamily. X-ray crystallographic determinations of several bacterial P450 enzymes in the 1990s (see Table 1 for a summary of structural data) have stimulated numerous attempts in modeling microsomal P450S such as human CYP3A4. The chapter 6 of the book “Guide to Cytochromes P450: structure and function” written by David F. V. Lewis reviews the current status of structural and modeling investigations of the P450 family (Lewis 2001). This review was however written just before the release of the first mammalian P450 structure (2C5), still today the only one mammalian template available.

TABLE 1

| CYP isoform | PDB code | | | No of | |
|---|---|---|---|---|---|
| crystallized | (resol.) | Organism | Function | residues | Reference |
| P450 cam | 3cpp | Pseudomonas | Camphor | 414 | (Poulos et al. |
| (complexed by | (1.9 Å) | Putida | Monooxygenase | | 1985) |
| CO + camphor) | | | | | (Raag and |
| | | | | | Poulos |
| | | | | | 1989) |
| P450 terp | 1cpt | Pseudomonas | Alpha-terpineol | 412 | (Hasemann et al. |
| | (2.3 Å) | sp. | hydroxylation | | 1994) |
| P450 BM3 | 2hpd | Bacillus megaterium | Fatty acid | 471 | (Ravichandran |
| | (2 Å) | | monooxygenase | | et al. 1993) |
| P450 cryF | 1oxa | Saccharopolyspora | Erythromycin | 403 | (Cupp-Vickery |
| (6-deoxyerythro- | (2.1 Å) | erythraea | biosynthesis 6S- | | and |
| nolide B bound) | | | hydroxylation of 6- | | Poulos 1995) |
| | | | deoxyerythronolide | | |
| | | | B | | |
| P450 nor | 1rom | Fusarium oxysporum | Nitric Oxide | 403 | (Park et al. 1997) |
| | (2 Å) | (denitrifying | Reductase | | |
| | | fungus) | | | |
| P450 2C5 | 1dt6 | (membrane-type | Progesterone 21- | 473 | (Williams et al. |
| | (3 Å) | Mammalian) | Hydroxylase | (487) | 2000) |
| | | Rabbit | | | |
| P450 CYP119 | 1f4t | Sulfobolus | unknown | 368 | (Yano et al. |
| 4-Phenylimidazole | (1.93 Å) | Solfactaricus | | | 2000) |
| Bound | | Thermophilic bact. | | | |
| P450 CYP51 | 1e9x | Mycobacterium | 14 α-sterol | 455 | (Podust et al. |
| 4-Phenylimidazole | (2.1 Å) | Tuberculosis | demethylase | (451) | 2001) |
| Bound | | | | | |

Table 1: the eight X-ray crystal structures of P450s available in 2002: six bacterial, one fungal (P450 nor), one mammalian (CYP2C5). The P450_{cam}, P450ₜₑᵣₚ, P450_{eryF}, P450ₙₒᵣ belong to class I P450s enzymes, whereas P450_{BM3} belongs to class II enzymes, like microsomal enzymes CYP2C5 and 3A. P450_{BM3} structure is therefore a priori more relevant to rebuilding a structural model of CYP3A, but since the CYP2CS X-Ray structure has been released, it became obvious that the structural homology between the other bacterial enzymes and microsomal enzymes was better than expected from the poor homology of primary structure (<25% identity). Then, the relevance of using class I and class II structures together for rebuilding models of class 11 P450s was no more questionable. In the two examples described in the present invention, the structural model of human CYP3A4 was rebuilt using the six first structures listed above, with no preference in the structural alignment, and the structural model of human CYP3A7 was rebuilt using four structures among those listed above with again no preference in the structural alignment, i.e. P450_{BM3}, P450 EryF, P450 2C5 and CYP51, one of the last published structural sets. CYP119 was not incorporated into the modeling process.

All the proposed models of CYP3A4 obtained by homology modeling are thus so far based on bacterial crystal structure templates: the first was proposed by Ferenczy and Morris and used the X-ray structure of bacterial P450_{cam}, as unique template structure (Ferenczy and Morris 1989). Another model was built later by David F. V. Lewis, using also a unique template structure, the P450_{BM3} structure, which was supposed to be more relevant as a template since this P450 was the only one class II enzyme with known three-dimensional structure (Lewis et al. 1996). A third model, based on a multiple structure template, was built by Szklarz and Halpert, using the four first X-ray crystal structures available P450_{cam}, P450ₜₑᵣₚ, P450_{eryF}, and P⁴⁵⁰_{BM3}. This four-bacterial template approach strategy is closer to our rebuilding strategy, but was still missing some relevance in the absence of a mammalian template. In our hands, the incorporation of the mammalian 2C5 crystal structure into rebuilding steps of models of cytochrome P450 3A proved to be decisive. Inclusion of 2C5 crystal structure had indeed a profound effect on the structural alignment with the five non-mammalian structures, resulting in a different topology of the active site and a marked divergence between the model and each individual template. The advantage of our multiple-template approach resides essentially in the availability of a final template that can be used to rebuild various mammalian cytochromes P450. Up to now there is no available crystal structure or structural model of human CYP3A5, CYP 3A7, CYP3A43 or other mammalian CYP3A.

More recently, two new bacterial P450 crystal structures emerged in the literature (Table 1): CYP51 (PDB code le9x), from Mycobacterium tuberculosis, that catalyzes the oxidative removal of 14α-methyl group from sterol precursors in sterol biosynthesis in yeast and fungi (ergosterol), plants (phytosterol) and mammals (cholesterol), for its potential in the design of antifungal agents (Podust et al. 2001). And CYP119 (PDB code lf4t), from the thermophilic archaeon Sulfolobus solfataricus, the first P450 identified in Archaea, for its interest in understanding the enhanced thermal stability of the structure, especially in the region of the active site (Yano et al. 2000). Those two structures have been shown to exhibit the typical bacterial P450 fold, with some exceptions in the topology. They have not been included as structural templates in the modeling steps of the CYP3A4 model described in example 1. The names of newly discovered P450s follow the now accepted nomenclature of David R. Nelson (Nelson 1999).

The protein databank (Brookhaven Protein Databank, http://www.rcsb.org/pdb/) currently indicates that there are 76 separate crystal structures available for the eight crystallized P450s, plus 7 crystal structures on hold (Sep. 1, 2002), the majority of which containing either bound substrates or inhibitors. Table 1 provides the relevant information about the structural templates used for human CYP3A model rebuilding. The idea behind homology modeling is that proteins belonging to the same functional class and showing a strong sequence identity, adopt a similar fold (review in (Hilbert et al. 1993)). Known analogous structures are then used to generate a template or parent structure for the unknown protein to be modeled. The reliability of the various methods employed depend mostly on the number of experimental 3D structures that can be aligned. Knowing that for pairs of distantly related proteins (with residue identity of about 20%) the regions having the same fold will represent less than half of each molecule, the regions where the folds differ will predominate, and the divergence of sequence must be compensated by a higher number of homologous proteins to align (Chothia and Lesk 1986). Below 50% of sequence identity, the deviation in structurally not conserved regions becomes significant, and loop regions are difficult to predict. It is generally accepted that below 20% of sequence identity, the prediction turns out to be hazardous, and fold assignment methods are best replaced by ab inilio methods, that ideally attempt to predict the native structure only from the primary sequence of the protein to be modeled. But produced models so far had the correct fold for only a few small protein domains (Sanchez et al. 2000).

The strategy of model rebuilding in the P450 family is strongly driven by the low degree of homology between bacterial and mammal cytochrome P450s (Table 2).

TABLE 2

| PDB | Swiss-Prot | CP37_{—} | CP34_{—} | CP51_{—} | CPXW_{—} | CPC5_{—} | CPXA_{—} | CPXL_{—} | CPXB_{—} | CPXJ_{—} | NOR_{—} |
|---|---|---|---|---|---|---|---|---|---|---|---|
| code | entry name | HUMAN | HUMAN | MYCTU | SULSO | RABIT | PSEPU | PSESP | BACME | SACER | FUSOX |
| n.s. | CP37_HUMAN | | | | | | | | | | |
| n.s. | CP34_HUMAN | | | | | | | | | | |
| 1E9X | CP51_MYCTU | 27.7% | 26.9% | | | | | | | | |
| | (CYP51) | 372 aa | 405 aa | | | | | | | | |
| 1F4T | CPXW_SULSO | 24.5% | 25.4% | 25.7% | | | | | | | |
| | (CYP119) | 330 aa | 410 aa | 385 aa | | | | | | | |
| 1DT6 | CPC5_RABIT | 27.9% | 28.4% | 23.4% | 23.5% | | | | | | |
| | (CYP2C5) | 481 aa | 497 aa | 427 aa | 344 aa | | | | | | |
| 3CPP | CPXA_PSEPU | 23.3% | 21.3% | 21.9% | 26.6% | 24.2% | | | | | |
| | (P450 cam) | 335 aa | 399 aa | 407 aa | 387 aa | 480 aa | | | | | |
| 1CPT | CPXL_PSESP | 24.8% | 24.4% | 27.8% | 29.3% | 24.4% | 27.4% | | | | |
| | (P450 terp) | 452 aa | 356 aa | 446 aa | 409 aa | 451 aa | 398 aa | | | | |
| 2HPD-A | CPXB_BACME | 31.8% | 29.9% | 27.1% | 24.5% | 22.7% | 23.1% | 24.0% | | | |
| | (P450 BM3) | 409 aa | 445 aa | 443 aa | 396 aa | 480 aa | 485 aa | 363 aa | | | |
| 1OXA | CPXJ_SACER | 25.5% | 26.0% | 27.7% | 30.8% | 24.4% | 24.0% | 28.6% | 22.6% | | |
| | (P450 eryF) | 415 aa | 334 aa | 423 aa | 396 aa | 443 aa | 391 aa | 420 aa | 389 aa | | |
| 1ROM | NOR_FUSOX | 23.7% | 22.9% | 23.1% | 27.7% | 21.4% | 29.0% | 31.5% | 23.0% | 32.7% | |
| | (P450 nor) | 354 aa | 415 aa | 442 aa | 379 aa | 351 aa | 379 aa | 409 aa | 265 aa | 395 aa | |

Table 2: Sequence identities between the various crystallized cytochrome P450s and human CYP3A4 and CYP3A7 using BLOSUM 62 matrix (source LALIGN, http://www.infobiogen.fr/services/analyseq/cgi-bin/lfastap_in.pl, algorithm of Huang and Miller LALIGN that finds the best local alignments between two sequences, version 2.1u03 April 2000, published in Adv. Appl. Math. 1991, 12: 373-381). The P450 BM3 structure, Swissprot code name CPXB_BACME, corresponds to the structure of a fusion protein of P450 and a reductase domain, so that it displays twice the number of residues.

Our global scheme, which steps are described hereafter, is founded on a combination of methods developed in the literature for different purposes in protein structure determination studies. The principle of the primary steps, until the generation of a correct alignment of P450 primary sequences, is described in Jean et al. 1997. The last steps are summarized in Loiseau 2002.

Therefore, in a first object, the invention relates to a method for designing a 3-dimentional (3-D) model of a protein, the 3-D representation of at least three family members has already been experimentally obtained, [said 3-D representation presenting similarities], comprising the steps of:

a. identification of common structural blocks (CSBs) among said members of said family,

b. alignment of the amino-acids primary sequence of said family members according to said structural similarities, represented by said CSBs, in order to obtain a first alignment,

c. alignment of said protein as compared on said first alignment, in order to obtain a second alignment, wherein:
i. alignment of said protein is performed in order to optimize the amino-acids alignment between said protein and said first alignment,
   when one or more consensus amino-acid exists in said aligned CSBs in said first alignment, and in the amino-acid sequence of said protein, said consensus amino-acids are anchors of said second alignment,
ii. no insertion or deletion of amino-acids can be performed in the aligned CSBs, wherein insertion or deletions are possible in out-of-block regions, if better to align the primary amino-acids sequences,

d. definition of the 3-D structure of CSBs of said protein, according to the 3-D structure of the CSBs of said family members,

e. definition of the global constraints (distance and angular constraints) derived from the comparisons of the structural templates in CSBs, and definition of the local constraints (distance and angular constraints) for the atoms of residues that are not structurally determined after step d. (that are not in the CSBs),

f. selection of rotamers,

g. determination of a family of 3-D model structures of said protein, taking into account said 3-D structure of CSBs obtained in step d., said global and local constraints defined in step e., and said rotamers defined in step f.,

h. optimization of said family of 3-D models obtained in step g., by

i discarding structures that present topological defects, and

ii recalculating 3-D structures by taking electrostatic forces into account, and performing the method again from step c. downward, with modifications in the alignment between the primary sequence of said protein and said first alignment, when the obtained model structures do not satisfactorily account for known mutations having biological effects.

In the present invention, the term “backbone atoms” refers to the C, N, Cα, and O atoms of a protein that are common to all amino acid building blocks or involved in the peptide linkage. When the protein structure is described as a trajectory in internal coordinates such as α, τ angles, or is a low-resolution crystallographic structure, backbone atoms stand only for Cα atoms of each residue.

In the present invention, the term “similarities” is used in the search for structural fragments conserved between the template proteins, that is fragments that have similar local trajectories in the backbone internal coordinate space. Two protein fragments have “similar” local trajectories when they are matched according to two adjustable parameters, the mesh and the margin (Jean et al. 1997).

In the present invention, the term “common structural blocks (CSB)” define the protein fragments of equal length that are found similar between all the template proteins in the internal coordinate representation.

In the present invention, the term “first alignment” refers to the alignment imposed by the CSBs, that is the structural alignment between template proteins defined by CSBs sequences. This alignment is totally independent on the primary sequence of the template proteins.

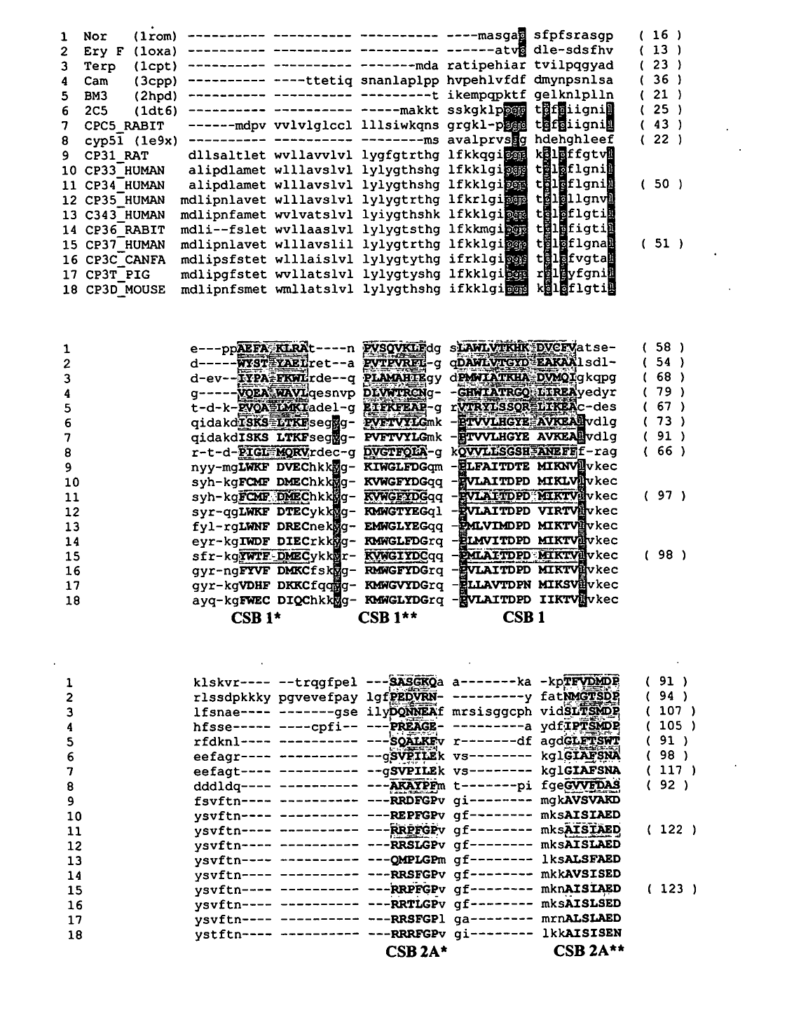

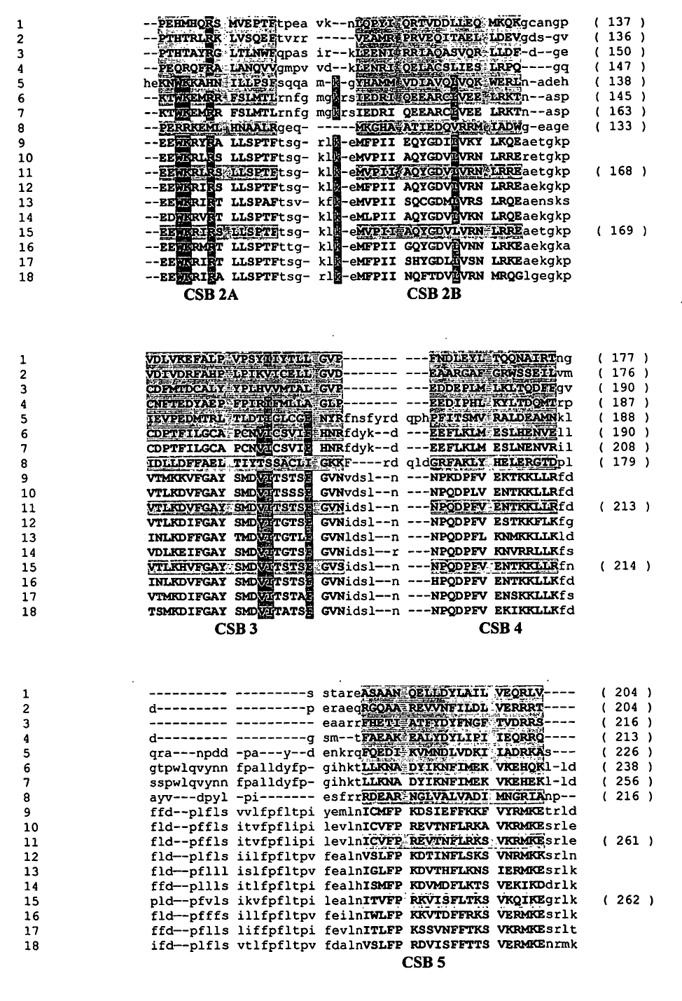

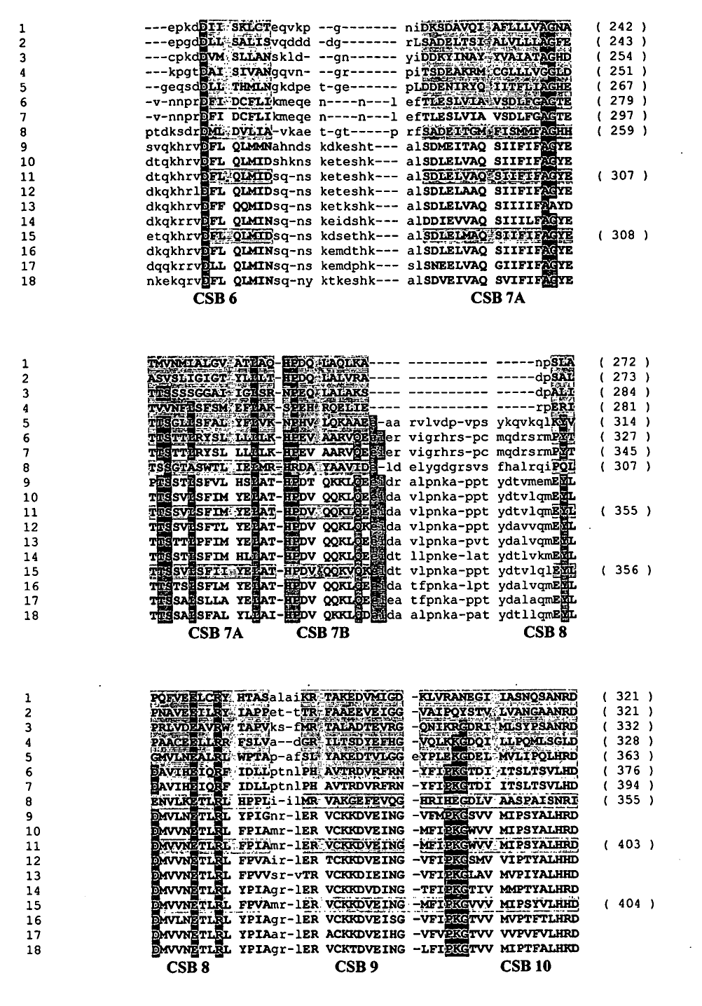

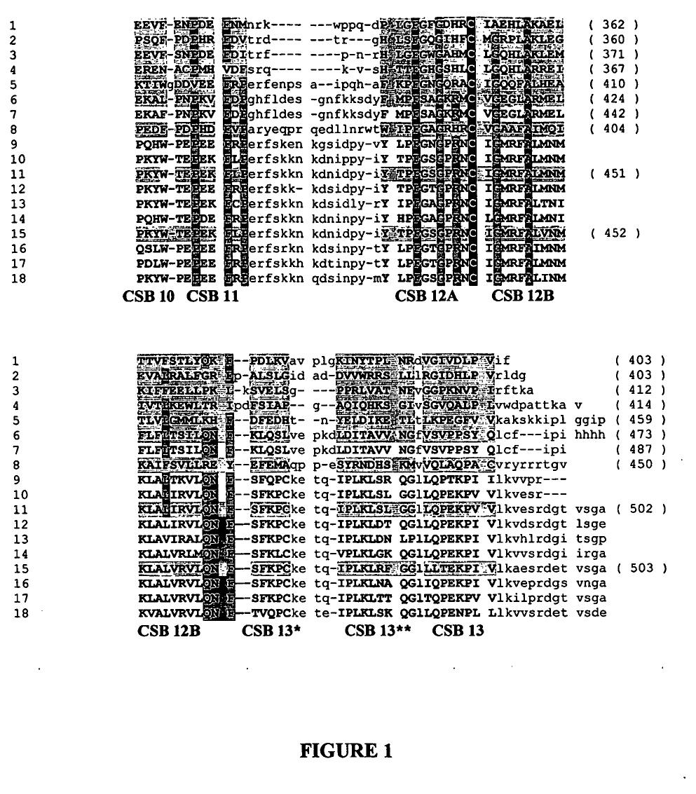

In the present invention, the term “out-of-block regions” designates all other protein fragments located out of and between the CSBs, i.e. that are not structurally conserved in the internal coordinate space. There is no information of sequence alignment for these regions (see in FIG. 1 regions that are not colored), since they are not relevant for structural conservation. Out-of-block regions are passively reconstructed with the rest of the structure during the calculation steps.

In the present invention, the term “global constraints” refers to geometric constraints that are assigned to atoms of residues from CSBs, and that can be derived by computing all distance or angle information available within CSBs or between CSB.

In the present invention, the term “local constraints” refers to loose structural constraints that are assigned to residues of out-of-block regions, in order to restrict their backbone conformation to allowed regions of the Ramachandran diagram.

In the present invention, the term “rotamers” defines the low energy side-chain conformations of residues. The use of a library of rotarners allows determining or modeling a structure with the most likely side-chain conformations, saving time and producing a structure that is more likely to be correct.

For identification of CSBs between all selected 3D structures:

CSBs define the common local folds found similar in the template proteins, and are used as building blocks to set up the fold of the model (results in Loiseau 2002). The non conserved regions, that can be parts of secondary structures or non-structured regions as loops, will be rebuilt with no initial structural information. For multiple alignment of crystalline P450s, on the basis of CSBs determination: Once the structurally conserved elements are identified, a first structural alignment between the template proteins is derived. The following step involves the localization of these elements in the target sequence. Sequence pairwise comparisons between selected crystal structures and CYP3A (Table 2) show low sequence identity, so that online tools of multiple alignment such as CLUSTALW or PHD (Heidelberg) fail to produce an clear-cut alignment. Instead, local alignment tools, such as that described in Jean et al. 1997, were used to match the CSB profile to the target sequence, where a matrix is slid along the sequence and a score of similarity (based on a standard matrix such as BLOSUM62) is calculated for each position. Online tools of multiple alignment such as CLUSTALW 1.8 can be further used for assessment of accuracy.

The target sequence of human cytochrome P450 3A is thus aligned against the multiple alignment obtained from the CSBs. This produces the key sequence alignment which allows the generation of the template structure used for the rebuilding of the various CYP3A models. Following steps involve:

1) Generation of distance and dihedral angles constraints.

2) Selection of rotamers for side chains in CSBs.

3) Calculation of a set of structures using DYANA software. Loops are rebuilt between CSBs.

4) Structure optimization under XPLOR software (Brünger 1992).

In a preferred embodiment, said 3-D representation of family members has been obtained by crystallography or NMR.

The alignment of said common structural blocks in steps b. and c. can be performed by use of the GOK software as described in Jean et al., 1997.

In addition, step d. is preferably performed according to the following rules:
i. at a given position, when residues are identical between all the template structures and the target sequence, the 3D coordinates of the reference residues are purely assigned to the target residue,
ii. When residues differ, only the coordinates of the backbone atoms are assigned (Cα), and sometimes Cβ or Cγ when they exist.

The definition of rebuilding global constraints in step e. is performed by using all available geometrical information intra- and inter-CSB (distances and angles), issued from the comparisons of the structural templates, each geometric constraint being defined as an interval. On another hand, the definition of local constraints for out-of-blocks residues is performed by analysis of the allowed regions in Ramachandran diagram.

Furthermore, distances and angles defining global constraints are preferably selected in step e. by the following rules:
i. all distances for which the lower boundary is less than 8 Å,
ii. all the distances involving at least one side-chain atom, to preserve the spatial arrangement between CSBs
iii. all the distances involving atoms of any active group such as an heme group, to fix as much as possible the neighborhood of said active group, such as an iron atom.

The distance of 8 Å is chosen in order to reduce drastically the total number of constraints to take into account in the computation, and to allow to excessively constrain the model.

Angular constraints are preferably selected in step e. by the following rule:
i. dihedral angles φ and ψ of all residues located in CSBs are defined as constraints, given by the average values of corresponding φ, ψ angles in said family members ± the calculated standard deviation.

To practice the method of the invention, rotamers in step f. can be selected from the couples according to the tables of Dunbrack and Karplus and step g. can be performed with the DYANA software, as described in Güntert et al, 1997.

In addition, the optimization in step h. comprises the use of the X-Plor software, as described in A. T. Brünger, X-PLOR, version 3.1.

The method according to the invention is particularly applicable to a cytochrome P450 subfamily 3A comprising mammal and human cytochromes P450 3A]

In a preferred embodiment, said mammal cytochrome P450 3A is selected from the group comprising CYP3A6 (SEQ ID N^{o}14), CYP3A12 (SEQ ID N^{o}16), CYP3A29 (SEQ ID N^{o}17) and CYP3A13 (SEQ ID N^{o}18).

In another preferred embodiment, said human cytochrome P450 subfamily 3A is selected from the group comprising CYP3A4 (SEQ ID N^{o}11), CYP3A7 (SEQ ID N^{o}15), CYP3A5 (SEQ ID N^{o}12) and CYP3A43 (SEQ ID N^{o}13).

The method is applicable as well to human cytochrome of the subfamily P450 3A4, wherein said family members that are used for performing said first alignment for designing a 3-D model of CYP3A4 are chosen from Nor (SEQ ID N^{o} 1), Ery F (SEQ ID N^{o} 2), terp (SEQ ID N^{o} 3), Cam (SEQ ID N^{o} 4), BM3 (SEQ ID N^{o} 5) and 2C5 (SEQ ID N^{o} 6).

The method is applicable as well to human cytochrome of the subfamily 3A7, wherein family members that are used for performing said first alignment for designing a 3-D model of CYP3A7 are chosen from Ery F (SEQ ID N^{o} 2), BM3 (SEQ ID N^{o} 5), CYP51 (SEQ ID N^{o} 8) and 2C5 (SEQ ID N^{o} 6).

The method is applicable as well to other mammalian cytochrome P450 3A isoforms.

In a second object, the invention is directed to 3-D structure model of a protein, obtained by the method as described above.

In a preferred embodiment, the protein is a cytochrome P450 subfamily 3A comprising mammal and human cytochromes P450 3A

In another preferred embodiment, the protein is selected from the group comprising CYP3A6 (SEQ ID N^{o}14), CYP3A12 (SEQ ID N^{o}16), CYP3A29 (SEQ ID N^{o}17) and CYP3A 13 (SEQ ID N^{o} 18).

In still another preferred embodiment, the protein is a human cytochrome P450 subfamily 3A selected from the group comprising CYP3A4 (SEQ ID N^{o} 11), CYP3A7 (SEQ ID N^{o} 15), CYP3A5 (SEQ ID N^{o} 12) and CYP3A43 (SEQ ID N^{o}13).

In still another preferred embodiment, the protein is a human cytochrome P450 3A4 or 3A7.

Regarding the rebuilt P450 3A4 model, the main residues involved in the recognition of the substrate are C97; R104; F101; F107; F247; F303 and C376.

More specifically, C97 and C376 are found in positions compatible with the formation of a disufide bridge allowing limited or enhanced flexibility of corresponding protein domains, while R104 is involved in the capture of the substrate that is close to the entrance site, and allows to accompany it to the active site. F303 is involved in the recognition of the substrate in the active site. F107; F247 and F303 are involved in the recognition at the modulation site responsible for positive regulation. Role of F303 in the active site has already been suggested by studies of Domanski et al. 1998 in the SRS 4 region (mutants 1300, F303, A304, and T308).

Features of this model comprise the 3-D atomic coordinates of Table 3.

Table 3

In a preferred embodiment, the residues C97; R104; F101; F107; F247; F303 and C376 are involved in the CYP 3A4 for the recognition and uptake of the substrate at the entry site, and its binding into the active site having the 3-D atomic coordinates of Table 3.

Regarding the P450 3A7 model, features comprise the 3-D atomic coordinates of Table 4.

Table 4

In a preferred embodiment the residues Q79; F102; R105; R106; F108; F248; F304 and E374 are involved in the CYP 3A7 for the recognition and uptake of the substrate at the entry site, and its binding into the active having the 3-D atomic coordinates of Table 4.

In a third object, the invention contemplates a method for designing a protein, biological functions of which are altered, comprising:

a) obtaining a 3-D model of said protein by the method as depicted above,

b) analyzing said model of step a., and determining the amino-acids that are putatively involved in the biological functions of said protein,

c) changing said amino-acids by mutating the corresponding nucleotides on the nucleic acid sequence coding for said protein, in order to obtain a mutated protein having altered properties.

In the present invention, the term “altered properties” means that the generated protein is altered in its enzymatic properties, such as the substrate recognition, the movements associated to the entrance or the exit of the substrate, the multiple binding at the active site, the allosteric behaviour, the electron transfer, the coupling to the P450 reductase.

In another object, the invention relates to a computer-assisted method for performing restrained dynamics docking of a substrate on an enzyme, a 3-D structure of which is available, comprising the steps:

j. determining a force field, and independently simulating the presence of said enzyme in said force field,

k. minimizing the potential energy (Ep) linked to said force field of said 3-D structure, wherein the spatial position of some atoms of said enzyme is fixed, and wherein the other atoms are mobile, by allowing mobility of the mobile atoms, by

i. simulating an increase in temperature (in order to give kinetic energy),

ii. and minimizing the potential energy by re-specifying the temperature as 0 Kelvin (K)

l. optionally repeating step k in order to obtain other Ep minima, wherein said Ep minima are such that the structure of the protein remains folded,

m. minimizing Ep in said force field of said 3-D structure, wherein all the atoms of the protein are mobile, by

i. simulating an increase in temperature (in order to give kinetic energy), and

ii. minimizing the potential energy by re-specifying the temperature as 0 Kelvin (K)

n. simulating, at 0 K the presence of said substrate next to said enzyme,

o. optionally generating a molecular dynamics simulation on said substrate and enzyme (simulating an increase in temperature, in order to allow mobility of the atoms)

p. generating some constraints to said substrate, in order to impose that it has interaction with said enzyme,

q. generating a molecular dynamics simulation on said substrate and enzyme, with said constraints imposed in step p.,

r. optionally, generating a molecular dynamics simulation on said substrate and enzyme without said constraints of step p.

In the present invention, the term “restrained dynamics docking” means a procedure by which the docking of the substrate is simulated using molecular dynamics (MD) simulations under constraints that are specified by the user.

In the present invention, the term “soft-restrained dynamics docking” refers to a restrained dynamics docking in which the substrate-protein distance constraints are loose, with force field parameters associated to the constraints as low as 1 or 2 Kcal/mol.

20 In the present invention, the term “constraints” when applied to substrate docking refers to a distance imposed between atoms of the protein, generally from the active site (such as atoms of the heme group), and atoms of the substrate. These distance restraints are defined as intervals, where the distance range is large enough to allow the free movement of the substrate within the active site.

In a preferred embodiment of this method for performing restrained dynamics docking, said fixed atoms in step k. are the backbone atoms N—Ca—CO in the first minimization step and only Ca in subsequent minimization steps.

In another preferred embodiment of this method, kinetic energy is simulated by temperature increase to about 100 K for about 5-20 ns.

The force field in step j. comprises forces linked to:

a. the distance between atoms,

b. the angles of valence,

c. the dihedral angles,

d. the deformation with regard to planar geometry,

e. the electrostatic field,

f. the Van der Waals forces,

g. hydrogen bonds.

The constraints in step p. are attraction constraints to force said substrate in the active site, and wherein said constraints are not prejudiced to the exact spatial conformation of the substrate in the active site. These constraints are final distance constraints between some atoms of said substrate and some atoms of amino-acids present in said active site.

In the present invention, the term “final distance constraints”, when applied to substrate docking, means distances imposed between atoms from the heme group (such as the iron atom), and atoms of the substrate. These distance contraints are defined as intervals, and are related to the final position of the substrate in the vicinity of the heme group before metabolization.

Preferably, step o. is performed with a simulated temperature of between about 15 and 50 K, step q. is performed with a simulated temperature of between about 15 and 50 K, and step r. is performed with a simulated temperature of between about 200 and 350 K.

This method is particularly suited for multispecific protein such as a cytochrome 36 P450 subfamily 3A comprising mammal and human cytochromes.

The cytochrome can be cytochrome P450 3A4 or any of all other P450 from the 3A subfamily, and said structure can be the structure obtained by the method of the invention described above, in particular the model structures which atomic coordinates are listed in Tables 3 and 4 for CYP3A4 and CYP3A7.

The substrate can be a small organic compound which size can range for example from MW 288 (testosterone) to MW 1203 (cyclosporine A).

In a preferred embodiment said substrate is testosterone.

In another object, the invention is aimed at a computer-assisted method for performing restrained dynamics docking of at least two substrates on an enzyme, a 3-D structure of which is available, consisting of performing the steps j, k, l, m, n, o, p, q and r depicted above with a first substrate and repeating said steps with a second substrate when the first substrate reaches an unconstrained state after molecular dynamics simulations.

The first and second substrates can be the same molecule or different molecules.

The first and second substrates can display either allosteric or synergistic effect.

This method can be practiced with substrates that are inhibitors (competitive, uncompetitive, non competitive) or display an inhibitor-base mechanism. It can also be practiced with an agonist and any molecule interfering with the biological function of the protein.

In preferred embodiments:
the first and second substrates are the same molecule.
the first and second substrates are different molecules.
the first and second substrates display an allosteric effect.
the first and second substrates display a synergistic effect.
at least one of the substrates is an inhibitor or display an inhibitor-based mechanism.
at least one of the substrates is an agonist.

In another embodiment, this method also embraces a successive repeat of the steps j, k, l, m, n, o, p, q and r depicted above with a 3^{rd}, 4^{th} or 5^{th} substrate, some of them being the same or different molecules.

In this method for performing restrained dynamics docking, said fixed atoms in step k. are the backbone atoms N—Cα-CO in the first minimization step and only Cα in subsequent minimization steps.

In addition, kinetic energy is simulated by temperature increase to about 100 K for about 5-20 ns.

The force field in step j. comprises preferably forces linked to

a. the distance between atoms,

b. the angles of valence,

c. the dihedral angles,

d. the deformation with regard to planar geometry,

e. the electrostatic field,

f. the Van der Waals forces,

g. hydrogen bonds.

The constraints in step p. are preferable attraction constraints to force said substrate in the active site, and wherein said constraints are not prejudiced to the exact spatial conformation of the substrate in the active site. These constraints are final distance constraints between some atoms of said substrate and some atoms of amino-acids present in said active site.

Preferably, step o. is performed with a simulated temperature of between about 15 and 50 K, step q. is performed with a simulated temperature of between about 15 and 50 K, and step r. is performed with a simulated temperature of between about 200 and 350 K.

This method is particularly suited for multispecific protein such as a cytochrome P450. The cytochrome can be cytochrome P450 3A4, or any of all other P450 of the 3A subfamily and said structure can be the structure obtained by the method of the invention described above, in particular the model structures which atomic coordinates are listed in Tables 3 and 4 for CYP3A4 and CYP3A7.

In a preferred embodiment:
said cytochrome is cytochrome P450 3A4, and said structure is the structure obtained by the above-described method, in particular the above-described model structure,
said first and second substrates are small organic compounds which size can range from MW 288 (testosterone) to MW 1203 (cyclosporine A),
said substrate is testosterone.

The invention is also directed to the use of the method for designing a 3-D model of a protein and to the computer-assisted method for performing restrained dynamics docking as mentioned above for screening, designing or identifying natural, unnatural substrates or substrate analogs, as well as inhibitors, activators or modulators of said enzyme.

Another object of the invention is the use of these methods for determining the effect of a first substrate on a second substrate, which can also be applied to pharmaceutical products.

The invention contemplates the use of these methods for determining the effect of a first bound testosterone molecule on the access of a second testosterone molecule as well as for determining the mutual effect of a testosterone molecule with alpha-naphtoflavone (αNF) molecule.

The invention is also directed to:
The use of the above described computer-assisted methods for determining the oxidative modification of the substrate according to the proximity to the heme of a part of the substrate to give rise to metabolite.
   The oxidized or reduced molecule derived from a given substrate modified after positioning at the right distance to the heme is called metabolite.
The use of the above described computer-assisted methods, for performing dynamic docking of the said metabolite, either in the absence or in the presence of the second substrate in the calculation.
The use of the above described computer-assisted methods for dynamic docking to compare the energy of the bound metabolite relatively to the energy of its parent substrate bound, in order to determine if the exit of the given metabolite from the enzyme is favored or not.
The use of the above described computer-assisted methods for dynamic docking to study the different exit pathways that are accessible to the metabolite, either in the absence or in the presence of the second substrate in the calculation.

The distance and angular constraints derived from CSBs common to the crystallized cytochromes P450 used as structural templates, are applied to conserved atoms of CSBs of the target protein. The DYANA software (Güntert et al. 1997) allows to rebuild directly the whole structure of the target protein on the basis of its primary sequence, by taking into account these geometric constraints. Out-of-blocks residues are rebuilt ab initio by selecting the most favorable solutions in terms of minimal global potential energy. As examples, actual tables 3 and 4 display the atomic coordinates of structural models obtained by applying DYANA calculation to target protein sequences CYP3A4 and CYP3A7 respectively.

TABLE 3
Providing the coordinates of the CYP3A4 model

| HEADER | CYP3A4 | | | | | | | | | | | | | | |
|---|---|---|---|---|---|---|---|---|---|---|---|---|---|---|---|
| TITLE | MODEL OF HUMAN CYTOCHROME P450 CYP3A4 | | | | | | | | | | | | | | |
| AUTHOR | N. LOISEAU, F. ANDRE, C. MINOLETTI, M. DELAFORGE | | | | | | | | | | | | | | |
| SEQRES | 1 | 452 | SER | TYR | HIS | LYS | GLY | PHE | CYS | MET | PHE | ASP | MET | GLU | CYS |
| SEQRES | 2 | 452 | HIS | LYS | LYS | TYR | GLY | LYS | VAL | TRP | GLY | PHE | TYR | ASP | GLY |
| SEQRES | 3 | 452 | GLN | GLN | PRO | VAL | LEU | ALA | ILE | THR | ASP | PRO | ASP | MET | ILE |
| SEQRES | 4 | 452 | LYS | THR | VAL | LEU | VAL | LYS | GLU | CYS | TYR | SER | VAL | PHE | THR |
| SEQRES | 5 | 452 | ASN | ARG | ARG | PRO | PHE | GLY | PRO | VAL | GLY | PHE | MET | LYS | SER |
| SEQRES | 6 | 452 | ALA | ILE | SER | ILE | ALA | GLU | ASP | GLU | GLU | TRP | LYS | ARG | LEU |
| SEQRES | 7 | 452 | ARG | SER | LEU | LEU | SER | PRO | THR | PHE | THR | SER | GLY | LYS | LEU |
| SEQRES | 8 | 452 | LYS | GLU | MET | VAL | PRO | ILE | ILE | ALA | GLN | TYR | GLY | ASP | VAL |
| SEQRES | 9 | 452 | LEU | VAL | ARG | ASN | LEU | ARG | ARG | GLU | ALA | GLU | THR | GLY | LYS |
| SEQRES | 10 | 452 | PRO | VAL | THR | LEU | LYS | ASP | VAL | PHE | GLY | ALA | TYR | SER | MET |
| SEQRES | 11 | 452 | ASP | VAL | ILE | THR | SER | THR | SER | PHE | GLY | VAL | ASN | ILE | ASP |
| SEQRES | 12 | 452 | SER | LEU | ASN | ASN | PRO | GLN | ASP | PRO | PHE | VAL | GLU | ASN | THR |
| SEQRES | 13 | 452 | LYS | LYS | LEU | LEU | ARG | PHE | ASP | PHE | LEU | ASP | PRO | PHE | PHE |
| SEQRES | 14 | 452 | LEU | SER | ILE | THR | VAL | PHE | PRO | PHE | LEU | ILE | PRO | ILE | LEU |
| SEQRES | 15 | 452 | GLU | VAL | LEU | ASN | ILE | CYS | VAL | PHE | PRO | ARG | GLU | VAL | THR |
| SEQRES | 16 | 452 | ASN | PHE | LEU | ARG | LYS | SER | VAL | LYS | ARG | MET | LYS | GLU | SER |
| SEQRES | 17 | 452 | ARG | LEU | GLU | ASP | THR | GLN | LYS | HIS | ARG | VAL | ASP | PHE | LEU |
| SEQRES | 18 | 452 | GLN | LEU | MET | ILE | ASP | SER | GLN | ASN | SER | LYS | GLU | THR | GLU |
| SEQRES | 19 | 452 | SER | HIS | LYS | ALA | LEU | SER | ASP | LEU | GLU | LEU | VAL | ALA | GLN |
| SEQRES | 20 | 452 | SER | ILE | ILE | PHE | ILE | PHE | ALA | GLY | TYR | GLU | THR | THR | SER |
| SEQRES | 21 | 452 | SER | VAL | LEU | SER | PHE | ILE | MET | TYR | GLU | LEU | ALA | THR | HIS |
| SEQRES | 22 | 452 | PRO | ASP | VAL | GLN | GLN | LYS | LEU | GLN | GLU | GLU | ILE | ASP | ALA |
| SEQRES | 23 | 452 | VAL | LEU | PRO | ASN | LYS | ALA | PRO | PRO | THR | TYR | ASP | THR | VAL |
| SEQRES | 24 | 452 | LEU | GLN | MET | GLU | TYR | LEU | ASP | MET | VAL | VAL | ASN | GLU | THR |
| SEQRES | 25 | 452 | LEU | ARG | LEU | PHE | PRO | ILE | ALA | MET | ARG | LEU | GLU | ARG | VAL |
| SEQRES | 26 | 452 | CYS | LYS | LYS | ASP | VAL | GLU | ILE | ASN | GLY | MET | PHE | ILE | PRO |
| SEQRES | 27 | 452 | LYS | GLY | TRP | VAL | VAL | MET | ILE | PRO | SER | TYR | ALA | LEU | HIS |
| SEQRES | 28 | 452 | ARG | ASP | PRO | LYS | TYR | TRP | THR | GLU | PRO | GLU | LYS | PHE | LEU |
| SEQRES | 29 | 452 | PRO | GLU | ARG | PHE | SER | LYS | LYS | ASN | LYS | ASP | ASN | ILE | ASP |
| SEQRES | 30 | 452 | PRO | TYR | ILE | TYR | THR | PRO | PHE | GLY | SER | GLY | PRO | ARG | ASN |
| SEQRES | 31 | 452 | CYS | ILE | GLY | MET | ARG | PHE | ALA | LEU | MET | ASN | MET | LYS | LEU |
| SEQRES | 32 | 452 | ALA | LEU | ILE | ARG | VAL | LEU | GLN | ASN | PHE | SER | PHE | LYS | PRO |
| SEQRES | 33 | 452 | CYS | LYS | GLU | THR | GLN | ILE | PRO | LEU | LYS | LEU | SER | LEU | GLY |
| SEQRES | 34 | 452 | GLY | LEU | LEU | GLN | PRO | GLU | LYS | PRO | VAL | VAL | LEU | LYS | VAL |
| SEQRES | 35 | 452 | GLU | SER | ARG | ASP | GLY | THR | VAL | SER | GLY | ALA | | | |
| HET | HEM | 600 | | | | | | | | | | | | | |

| HETNAM | HEM | HEME | | | | |
|---|---|---|---|---|---|---|
| HETSYN | HEM | 3,7,12,17-TETRAMETHYL-8,13-DIVINYL-2,18-PORPHINEDIPROPIONIC ACID | | | | |
| FORMUL | HEM | C34 | H34 | N4 | O4 | FE1 |

| | | | | | | | | | | |
|---|---|---|---|---|---|---|---|---|---|---|
| ATOM | 1 | N | SER | 51 | 9.999 | −1.760 | −4.543 | 1.00 | 0.00 | 3A4 |
| ATOM | 2 | CA | SER | 51 | 10.718 | −0.477 | −4.293 | 1.00 | 0.00 | 3A4 |
| ATOM | 3 | CB | SER | 51 | 9.949 | 0.731 | −4.939 | 1.00 | 0.00 | 3A4 |
| ATOM | 4 | OG | SER | 51 | 8.601 | 0.876 | −4.493 | 1.00 | 0.00 | 3A4 |
| ATOM | 5 | C | SER | 51 | 10.962 | −0.281 | −2.815 | 1.00 | 0.00 | 3A4 |
| ATOM | 6 | O | SER | 51 | 10.277 | −0.855 | −1.969 | 1.00 | 0.00 | 3A4 |
| ATOM | 7 | N | TYR | 52 | 11.974 | 0.569 | −2.482 | 1.00 | 0.00 | 3A4 |
| ATOM | 8 | CA | TYR | 52 | 12.433 | 0.860 | −1.131 | 1.00 | 0.00 | 3A4 |
| ATOM | 9 | CB | TYR | 52 | 13.983 | 0.783 | −0.979 | 1.00 | 0.00 | 3A4 |
| ATOM | 10 | CG | TYR | 52 | 14.459 | −0.572 | −1.430 | 1.00 | 0.00 | 3A4 |
| ATOM | 11 | CD1 | TYR | 52 | 14.143 | −1.732 | −0.699 | 1.00 | 0.00 | 3A4 |
| ATOM | 12 | CD2 | TYR | 52 | 15.210 | −0.701 | −2.611 | 1.00 | 0.00 | 3A4 |
| ATOM | 13 | CE1 | TYR | 52 | 14.574 | −2.994 | −1.138 | 1.00 | 0.00 | 3A4 |
| ATOM | 14 | CE2 | TYR | 52 | 15.651 | −1.954 | −3.052 | 1.00 | 0.00 | 3A4 |
| ATOM | 15 | CZ | TYR | 52 | 15.334 | −3.105 | −2.314 | 1.00 | 0.00 | 3A4 |
| ATOM | 16 | OH | TYR | 52 | 15.784 | −4.370 | −2.751 | 1.00 | 0.00 | 3A4 |
| ATOM | 17 | C | TYR | 52 | 11.971 | 2.246 | −0.755 | 1.00 | 0.00 | 3A4 |
| ATOM | 18 | O | TYR | 52 | 12.178 | 3.208 | −1.494 | 1.00 | 0.00 | 3A4 |
| ATOM | 19 | N | HIS | 53 | 11.321 | 2.350 | 0.429 | 1.00 | 0.00 | 3A4 |
| ATOM | 20 | CA | HIS | 53 | 10.747 | 3.568 | 0.953 | 1.00 | 0.00 | 3A4 |
| ATOM | 21 | ND1 | HIS | 53 | 7.857 | 1.850 | 1.039 | 1.00 | 0.00 | 3A4 |
| ATOM | 22 | CG | HIS | 53 | 8.485 | 2.636 | 0.097 | 1.00 | 0.00 | 3A4 |
| ATOM | 23 | CB | HIS | 53 | 9.322 | 3.861 | 0.386 | 1.00 | 0.00 | 3A4 |
| ATOM | 24 | NE2 | HIS | 53 | 7.438 | 0.923 | −0.936 | 1.00 | 0.00 | 3A4 |
| ATOM | 25 | CD2 | HIS | 53 | 8.216 | 2.053 | −1.104 | 1.00 | 0.00 | 3A4 |
| ATOM | 26 | CE1 | HIS | 53 | 7.247 | 0.842 | 0.368 | 1.00 | 0.00 | 3A4 |
| ATOM | 27 | C | HIS | 53 | 10.751 | 3.386 | 2.448 | 1.00 | 0.00 | 3A4 |
| ATOM | 28 | O | HIS | 53 | 9.715 | 3.183 | 3.080 | 1.00 | 0.00 | 3A4 |
| ATOM | 29 | N | LYS | 54 | 11.973 | 3.461 | 3.043 | 1.00 | 0.00 | 3A4 |
| ATOM | 30 | CA | LYS | 54 | 12.265 | 3.262 | 4.451 | 1.00 | 0.00 | 3A4 |
| ATOM | 31 | CB | LYS | 54 | 13.401 | 2.217 | 4.659 | 1.00 | 0.00 | 3A4 |
| ATOM | 32 | CG | LYS | 54 | 13.053 | 0.819 | 4.120 | 1.00 | 0.00 | 3A4 |
| ATOM | 33 | CD | LYS | 54 | 14.220 | −0.183 | 4.148 | 1.00 | 0.00 | 3A4 |
| ATOM | 34 | CE | LYS | 54 | 14.793 | −0.500 | 5.540 | 1.00 | 0.00 | 3A4 |
| ATOM | 35 | NZ | LYS | 54 | 13.763 | −1.087 | 6.431 | 1.00 | 0.00 | 3A4 |
| ATOM | 36 | C | LYS | 54 | 12.660 | 4.603 | 5.025 | 1.00 | 0.00 | 3A4 |
| ATOM | 37 | O | LYS | 54 | 13.829 | 4.851 | 5.317 | 1.00 | 0.00 | 3A4 |
| ATOM | 38 | N | GLY | 55 | 11.656 | 5.510 | 5.181 | 1.00 | 0.00 | 3A4 |
| ATOM | 39 | CA | GLY | 55 | 11.819 | 6.901 | 5.560 | 1.00 | 0.00 | 3A4 |
| ATOM | 40 | C | GLY | 55 | 11.464 | 7.113 | 7.003 | 1.00 | 0.00 | 3A4 |
| ATOM | 41 | O | GLY | 55 | 10.305 | 7.002 | 7.400 | 1.00 | 0.00 | 3A4 |
| ATOM | 42 | N | PHE | 56 | 12.502 | 7.439 | 7.817 | 1.00 | 0.00 | 3A4 |
| ATOM | 43 | CA | PHE | 56 | 12.451 | 7.714 | 9.250 | 1.00 | 0.00 | 3A4 |
| ATOM | 44 | CB | PHE | 56 | 13.646 | 7.051 | 10.010 | 1.00 | 0.00 | 3A4 |
| ATOM | 45 | CG | PHE | 56 | 13.966 | 5.681 | 9.448 | 1.00 | 0.00 | 3A4 |
| ATOM | 46 | CD1 | PHE | 56 | 15.250 | 5.415 | 8.926 | 1.00 | 0.00 | 3A4 |
| ATOM | 47 | CD2 | PHE | 56 | 12.987 | 4.669 | 9.370 | 1.00 | 0.00 | 3A4 |
| ATOM | 48 | CE1 | PHE | 56 | 15.543 | 4.185 | 8.320 | 1.00 | 0.00 | 3A4 |
| ATOM | 49 | CE2 | PHE | 56 | 13.271 | 3.445 | 8.747 | 1.00 | 0.00 | 3A4 |
| ATOM | 50 | CZ | PHE | 56 | 14.550 | 3.205 | 8.224 | 1.00 | 0.00 | 3A4 |
| ATOM | 51 | C | PHE | 56 | 12.446 | 9.211 | 9.542 | 1.00 | 0.00 | 3A4 |
| ATOM | 52 | O | PHE | 56 | 11.957 | 9.670 | 10.570 | 1.00 | 0.00 | 3A4 |
| ATOM | 53 | N | CYS | 57 | 12.971 | 10.054 | 8.624 | 1.00 | 0.00 | 3A4 |
| ATOM | 54 | CA | CYS | 57 | 13.048 | 11.500 | 8.782 | 1.00 | 0.00 | 3A4 |
| ATOM | 55 | CB | CYS | 57 | 13.902 | 12.161 | 7.665 | 1.00 | 0.00 | 3A4 |
| ATOM | 56 | SG | CYS | 57 | 15.312 | 11.121 | 7.176 | 1.00 | 0.00 | 3A4 |
| ATOM | 57 | C | CYS | 57 | 11.705 | 12.171 | 8.760 | 1.00 | 0.00 | 3A4 |
| ATOM | 58 | O | CYS | 57 | 11.446 | 13.160 | 9.434 | 1.00 | 0.00 | 3A4 |
| ATOM | 59 | N | MET | 58 | 10.778 | 11.585 | 7.975 | 1.00 | 0.00 | 3A4 |
| ATOM | 60 | CA | MET | 58 | 9.402 | 12.000 | 7.870 | 1.00 | 0.00 | 3A4 |
| ATOM | 61 | CB | MET | 58 | 8.715 | 11.289 | 6.694 | 1.00 | 0.00 | 3A4 |
| ATOM | 62 | CG | MET | 58 | 9.360 | 11.641 | 5.343 | 1.00 | 0.00 | 3A4 |
| ATOM | 63 | SD | MET | 58 | 8.469 | 11.004 | 3.887 | 1.00 | 0.00 | 3A4 |
| ATOM | 64 | CE | MET | 58 | 8.922 | 9.256 | 4.080 | 1.00 | 0.00 | 3A4 |
| ATOM | 65 | C | MET | 58 | 8.612 | 11.706 | 9.122 | 1.00 | 0.00 | 3A4 |
| ATOM | 66 | O | MET | 58 | 7.740 | 12.480 | 9.487 | 1.00 | 0.00 | 3A4 |
| ATOM | 67 | N | PHE | 59 | 8.963 | 10.612 | 9.844 | 1.00 | 0.00 | 3A4 |
| ATOM | 68 | CA | PHE | 59 | 8.394 | 10.230 | 11.122 | 1.00 | 0.00 | 3A4 |
| ATOM | 69 | CB | PHE | 59 | 8.732 | 8.752 | 11.449 | 1.00 | 0.00 | 3A4 |
| ATOM | 70 | CG | PHE | 59 | 7.865 | 8.152 | 12.538 | 1.00 | 0.00 | 3A4 |
| ATOM | 71 | CD1 | PHE | 59 | 6.477 | 8.002 | 12.350 | 1.00 | 0.00 | 3A4 |
| ATOM | 72 | CD2 | PHE | 59 | 8.430 | 7.747 | 13.764 | 1.00 | 0.00 | 3A4 |
| ATOM | 73 | CE1 | PHE | 59 | 5.671 | 7.462 | 13.364 | 1.00 | 0.00 | 3A4 |
| ATOM | 74 | CE2 | PHE | 59 | 7.627 | 7.208 | 14.780 | 1.00 | 0.00 | 3A4 |
| ATOM | 75 | CZ | PHE | 59 | 6.247 | 7.065 | 14.579 | 1.00 | 0.00 | 3A4 |
| ATOM | 76 | C | PHE | 59 | 8.881 | 11.119 | 12.255 | 1.00 | 0.00 | 3A4 |
| ATOM | 77 | O | PHE | 59 | 8.110 | 11.479 | 13.139 | 1.00 | 0.00 | 3A4 |
| ATOM | 78 | N | ASP | 60 | 10.162 | 11.568 | 12.226 | 1.00 | 0.00 | 3A4 |
| ATOM | 79 | CA | ASP | 60 | 10.690 | 12.515 | 13.200 | 1.00 | 0.00 | 3A4 |
| ATOM | 80 | CB | ASP | 60 | 12.225 | 12.651 | 13.131 | 1.00 | 0.00 | 3A4 |
| ATOM | 81 | CG | ASP | 60 | 12.906 | 11.313 | 13.450 | 1.00 | 0.00 | 3A4 |
| ATOM | 82 | OD1 | ASP | 60 | 12.613 | 10.736 | 14.532 | 1.00 | 0.00 | 3A4 |
| ATOM | 83 | OD2 | ASP | 60 | 13.743 | 10.859 | 12.624 | 1.00 | 0.00 | 3A4 |
| ATOM | 84 | C | ASP | 60 | 10.102 | 13.900 | 13.031 | 1.00 | 0.00 | 3A4 |
| ATOM | 85 | O | ASP | 60 | 9.841 | 14.603 | 14.000 | 1.00 | 0.00 | 3A4 |
| ATOM | 86 | N | MET | 61 | 9.809 | 14.318 | 11.780 | 1.00 | 0.00 | 3A4 |
| ATOM | 87 | CA | MET | 61 | 9.130 | 15.564 | 11.481 | 1.00 | 0.00 | 3A4 |
| ATOM | 88 | CB | MET | 61 | 9.224 | 15.881 | 9.987 | 1.00 | 0.00 | 3A4 |
| ATOM | 89 | CG | MET | 61 | 10.590 | 16.349 | 9.497 | 1.00 | 0.00 | 3A4 |
| ATOM | 90 | SD | MET | 61 | 10.659 | 16.502 | 7.686 | 1.00 | 0.00 | 3A4 |
| ATOM | 91 | CE | MET | 61 | 12.223 | 17.411 | 7.689 | 1.00 | 0.00 | 3A4 |
| ATOM | 92 | C | MET | 61 | 7.653 | 15.548 | 11.870 | 1.00 | 0.00 | 3A4 |
| ATOM | 93 | O | MET | 61 | 7.073 | 16.551 | 12.274 | 1.00 | 0.00 | 3A4 |
| ATOM | 94 | N | GLU | 62 | 7.012 | 14.358 | 11.819 | 1.00 | 0.00 | 3A4 |
| ATOM | 95 | CA | GLU | 62 | 5.653 | 14.159 | 12.272 | 1.00 | 0.00 | 3A4 |
| ATOM | 96 | CB | GLU | 62 | 5.077 | 12.770 | 11.933 | 1.00 | 0.00 | 3A4 |
| ATOM | 97 | CG | GLU | 62 | 4.640 | 12.657 | 10.462 | 1.00 | 0.00 | 3A4 |
| ATOM | 98 | CD | GLU | 62 | 4.351 | 11.190 | 10.120 | 1.00 | 0.00 | 3A4 |
| ATOM | 99 | OE1 | GLU | 62 | 3.429 | 10.603 | 10.748 | 1.00 | 0.00 | 3A4 |
| ATOM | 100 | OE2 | GLU | 62 | 5.048 | 10.637 | 9.227 | 1.00 | 0.00 | 3A4 |
| ATOM | 101 | C | GLU | 62 | 5.522 | 14.338 | 13.751 | 1.00 | 0.00 | 3A4 |
| ATOM | 102 | O | GLU | 62 | 4.508 | 14.819 | 14.225 | 1.00 | 0.00 | 3A4 |
| ATOM | 103 | N | CYS | 63 | 6.605 | 14.104 | 14.522 | 1.00 | 0.00 | 3A4 |
| ATOM | 104 | CA | CYS | 63 | 6.664 | 14.441 | 15.921 | 1.00 | 0.00 | 3A4 |
| ATOM | 105 | CB | CYS | 63 | 7.902 | 13.805 | 16.588 | 1.00 | 0.00 | 3A4 |
| ATOM | 106 | SG | CYS | 63 | 7.977 | 11.995 | 16.382 | 1.00 | 0.00 | 3A4 |
| ATOM | 107 | C | CYS | 63 | 6.526 | 15.952 | 16.211 | 1.00 | 0.00 | 3A4 |
| ATOM | 108 | O | CYS | 63 | 6.277 | 16.322 | 17.335 | 1.00 | 0.00 | 3A4 |
| ATOM | 109 | N | HIS | 64 | 6.436 | 16.853 | 15.182 | 1.00 | 0.00 | 3A4 |
| ATOM | 110 | CA | HIS | 64 | 5.487 | 17.970 | 15.236 | 1.00 | 0.00 | 3A4 |
| ATOM | 111 | ND1 | HIS | 64 | 4.140 | 20.440 | 16.744 | 1.00 | 0.00 | 3A4 |
| ATOM | 112 | CG | HIS | 64 | 5.232 | 20.460 | 15.904 | 1.00 | 0.00 | 3A4 |
| ATOM | 113 | CB | HIS | 64 | 6.151 | 19.284 | 15.666 | 1.00 | 0.00 | 3A4 |
| ATOM | 114 | NE2 | HIS | 64 | 4.280 | 22.506 | 15.939 | 1.00 | 0.00 | 3A4 |
| ATOM | 115 | CD2 | HIS | 64 | 5.301 | 21.731 | 15.420 | 1.00 | 0.00 | 3A4 |
| ATOM | 116 | CE1 | HIS | 64 | 3.608 | 21.687 | 16.726 | 1.00 | 0.00 | 3A4 |
| ATOM | 117 | C | HIS | 64 | 4.780 | 18.138 | 13.901 | 1.00 | 0.00 | 3A4 |
| ATOM | 118 | O | HIS | 64 | 3.812 | 17.438 | 13.610 | 1.00 | 0.00 | 3A4 |
| ATOM | 119 | N | LYS | 65 | 5.240 | 19.107 | 13.073 | 1.00 | 0.00 | 3A4 |
| ATOM | 120 | CA | LYS | 65 | 4.699 | 19.426 | 11.766 | 1.00 | 0.00 | 3A4 |
| ATOM | 121 | CB | LYS | 65 | 3.264 | 20.052 | 11.761 | 1.00 | 0.00 | 3A4 |
| ATOM | 122 | CG | LYS | 65 | 2.994 | 21.171 | 12.783 | 1.00 | 0.00 | 3A4 |
| ATOM | 123 | CD | LYS | 65 | 1.557 | 21.704 | 12.722 | 1.00 | 0.00 | 3A4 |
| ATOM | 124 | CE | LYS | 65 | 1.220 | 22.723 | 13.820 | 1.00 | 0.00 | 3A4 |
| ATOM | 125 | NZ | LYS | 65 | 2.052 | 23.944 | 13.700 | 1.00 | 0.00 | 3A4 |
| ATOM | 126 | C | LYS | 65 | 5.705 | 20.365 | 11.146 | 1.00 | 0.00 | 3A4 |
| ATOM | 127 | O | LYS | 65 | 5.958 | 20.315 | 9.944 | 1.00 | 0.00 | 3A4 |
| ATOM | 128 | N | LYS | 66 | 6.332 | 21.227 | 12.000 | 1.00 | 0.00 | 3A4 |
| ATOM | 129 | CA | LYS | 66 | 7.517 | 22.020 | 11.730 | 1.00 | 0.00 | 3A4 |
| ATOM | 130 | CB | LYS | 66 | 7.373 | 23.548 | 12.037 | 1.00 | 0.00 | 3A4 |
| ATOM | 131 | CG | LYS | 66 | 6.519 | 24.352 | 11.039 | 1.00 | 0.00 | 3A4 |
| ATOM | 132 | CD | LYS | 66 | 5.001 | 24.175 | 11.175 | 1.00 | 0.00 | 3A4 |
| ATOM | 133 | CE | LYS | 66 | 4.191 | 25.119 | 10.278 | 1.00 | 0.00 | 3A4 |
| ATOM | 134 | NZ | LYS | 66 | 2.736 | 24.881 | 10.442 | 1.00 | 0.00 | 3A4 |
| ATOM | 135 | C | LYS | 66 | 8.551 | 21.370 | 12.620 | 1.00 | 0.00 | 3A4 |
| ATOM | 136 | O | LYS | 66 | 9.100 | 20.332 | 12.253 | 1.00 | 0.00 | 3A4 |
| ATOM | 137 | N | TYR | 67 | 8.772 | 21.934 | 13.838 | 1.00 | 0.00 | 3A4 |
| ATOM | 138 | CA | TYR | 67 | 9.441 | 21.263 | 14.936 | 1.00 | 0.00 | 3A4 |
| ATOM | 139 | CB | TYR | 67 | 11.005 | 21.348 | 14.926 | 1.00 | 0.00 | 3A4 |
| ATOM | 140 | CG | TYR | 67 | 11.555 | 20.265 | 14.028 | 1.00 | 0.00 | 3A4 |
| ATOM | 141 | CD1 | TYR | 67 | 11.325 | 18.909 | 14.336 | 1.00 | 0.00 | 3A4 |
| ATOM | 142 | CD2 | TYR | 67 | 12.250 | 20.569 | 12.847 | 1.00 | 0.00 | 3A4 |
| ATOM | 143 | CE1 | TYR | 67 | 11.798 | 17.884 | 13.506 | 1.00 | 0.00 | 3A4 |
| ATOM | 144 | CE2 | TYR | 67 | 12.720 | 19.550 | 12.006 | 1.00 | 0.00 | 3A4 |
| ATOM | 145 | CZ | TYR | 67 | 12.507 | 18.207 | 12.342 | 1.00 | 0.00 | 3A4 |
| ATOM | 146 | OH | TYR | 67 | 13.010 | 17.184 | 11.509 | 1.00 | 0.00 | 3A4 |
| ATOM | 147 | C | TYR | 67 | 8.880 | 21.880 | 16.194 | 1.00 | 0.00 | 3A4 |
| ATOM | 148 | O | TYR | 67 | 8.905 | 23.092 | 16.404 | 1.00 | 0.00 | 3A4 |
| ATOM | 149 | N | GLY | 68 | 8.343 | 20.969 | 17.043 | 1.00 | 0.00 | 3A4 |
| ATOM | 150 | CA | GLY | 68 | 7.620 | 21.161 | 18.278 | 1.00 | 0.00 | 3A4 |
| ATOM | 151 | C | GLY | 68 | 8.251 | 20.139 | 19.164 | 1.00 | 0.00 | 3A4 |
| ATOM | 152 | O | GLY | 68 | 8.314 | 18.954 | 18.839 | 1.00 | 0.00 | 3A4 |
| ATOM | 153 | N | LYS | 69 | 8.864 | 20.642 | 20.260 | 1.00 | 0.00 | 3A4 |
| ATOM | 154 | CA | LYS | 69 | 10.301 | 20.592 | 20.404 | 1.00 | 0.00 | 3A4 |
| ATOM | 155 | CB | LYS | 69 | 10.795 | 21.997 | 20.802 | 1.00 | 0.00 | 3A4 |
| ATOM | 156 | CG | LYS | 69 | 10.332 | 23.112 | 19.844 | 1.00 | 0.00 | 3A4 |
| ATOM | 157 | CD | LYS | 69 | 10.770 | 24.527 | 20.225 | 1.00 | 0.00 | 3A4 |
| ATOM | 158 | CE | LYS | 69 | 9.876 | 25.161 | 21.300 | 1.00 | 0.00 | 3A4 |
| ATOM | 159 | NZ | LYS | 69 | 10.236 | 26.582 | 21.514 | 1.00 | 0.00 | 3A4 |
| ATOM | 160 | C | LYS | 69 | 10.811 | 19.541 | 21.360 | 1.00 | 0.00 | 3A4 |
| ATOM | 161 | O | LYS | 69 | 11.914 | 19.033 | 21.177 | 1.00 | 0.00 | 3A4 |
| ATOM | 162 | N | VAL | 70 | 10.033 | 19.200 | 22.410 | 1.00 | 0.00 | 3A4 |
| ATOM | 163 | CA | VAL | 70 | 10.427 | 18.227 | 23.408 | 1.00 | 0.00 | 3A4 |
| ATOM | 164 | CB | VAL | 70 | 10.984 | 18.837 | 24.696 | 1.00 | 0.00 | 3A4 |
| ATOM | 165 | CG1 | VAL | 70 | 12.504 | 18.835 | 24.571 | 1.00 | 0.00 | 3A4 |
| ATOM | 166 | CG2 | VAL | 70 | 10.344 | 20.206 | 25.037 | 1.00 | 0.00 | 3A4 |
| ATOM | 167 | C | VAL | 70 | 9.212 | 17.394 | 23.695 | 1.00 | 0.00 | 3A4 |
| ATOM | 168 | O | VAL | 70 | 8.176 | 17.901 | 24.111 | 1.00 | 0.00 | 3A4 |
| ATOM | 169 | N | TRP | 71 | 9.337 | 16.067 | 23.484 | 1.00 | 0.00 | 3A4 |
| ATOM | 170 | CA | TRP | 71 | 8.255 | 15.133 | 23.636 | 1.00 | 0.00 | 3A4 |
| ATOM | 171 | CB | TRP | 71 | 7.614 | 14.787 | 22.260 | 1.00 | 0.00 | 3A4 |
| ATOM | 172 | CG | TRP | 71 | 6.390 | 13.874 | 22.229 | 1.00 | 0.00 | 3A4 |
| ATOM | 173 | CD2 | TRP | 71 | 5.135 | 14.145 | 22.880 | 1.00 | 0.00 | 3A4 |
| ATOM | 174 | CD1 | TRP | 71 | 6.251 | 12.660 | 21.615 | 1.00 | 0.00 | 3A4 |
| ATOM | 175 | NE1 | TRP | 71 | 4.996 | 12.146 | 21.846 | 1.00 | 0.00 | 3A4 |
| ATOM | 176 | CE2 | TRP | 71 | 4.292 | 13.040 | 22.624 | 1.00 | 0.00 | 3A4 |
| ATOM | 177 | CE3 | TRP | 71 | 4.695 | 15.227 | 23.643 | 1.00 | 0.00 | 3A4 |
| ATOM | 178 | CZ2 | TRP | 71 | 2.995 | 12.995 | 23.131 | 1.00 | 0.00 | 3A4 |
| ATOM | 179 | CZ3 | TRP | 71 | 3.388 | 15.180 | 24.153 | 1.00 | 0.00 | 3A4 |
| ATOM | 180 | CH2 | TRP | 71 | 2.551 | 14.080 | 23.902 | 1.00 | 0.00 | 3A4 |
| ATOM | 181 | C | TRP | 71 | 8.828 | 13.911 | 24.261 | 1.00 | 0.00 | 3A4 |
| ATOM | 182 | O | TRP | 71 | 9.989 | 13.584 | 24.059 | 1.00 | 0.00 | 3A4 |
| ATOM | 183 | N | GLY | 72 | 7.987 | 13.188 | 25.027 | 1.00 | 0.00 | 3A4 |
| ATOM | 184 | CA | GLY | 72 | 8.345 | 11.936 | 25.647 | 1.00 | 0.00 | 3A4 |
| ATOM | 185 | C | GLY | 72 | 7.812 | 10.809 | 24.804 | 1.00 | 0.00 | 3A4 |
| ATOM | 186 | O | GLY | 72 | 6.679 | 10.850 | 24.336 | 1.00 | 0.00 | 3A4 |
| ATOM | 187 | N | PHE | 73 | 8.645 | 9.773 | 24.586 | 1.00 | 0.00 | 3A4 |
| ATOM | 188 | CA | PHE | 73 | 8.334 | 8.591 | 23.820 | 1.00 | 0.00 | 3A4 |
| ATOM | 189 | CB | PHE | 73 | 9.278 | 8.443 | 22.586 | 1.00 | 0.00 | 3A4 |
| ATOM | 190 | CG | PHE | 73 | 8.893 | 7.320 | 21.647 | 1.00 | 0.00 | 3A4 |
| ATOM | 191 | CD1 | PHE | 73 | 9.691 | 6.162 | 21.549 | 1.00 | 0.00 | 3A4 |
| ATOM | 192 | CD2 | PHE | 73 | 7.717 | 7.398 | 20.877 | 1.00 | 0.00 | 3A4 |
| ATOM | 193 | CE1 | PHE | 73 | 9.320 | 5.105 | 20.707 | 1.00 | 0.00 | 3A4 |
| ATOM | 194 | CE2 | PHE | 73 | 7.344 | 6.343 | 20.030 | 1.00 | 0.00 | 3A4 |
| ATOM | 195 | CZ | PHE | 73 | 8.146 | 5.196 | 19.947 | 1.00 | 0.00 | 3A4 |
| ATOM | 196 | C | PHE | 73 | 8.515 | 7.451 | 24.788 | 1.00 | 0.00 | 3A4 |
| ATOM | 197 | O | PHE | 73 | 9.257 | 7.543 | 25.757 | 1.00 | 0.00 | 3A4 |
| ATOM | 198 | N | TYR | 74 | 7.826 | 6.328 | 24.540 | 1.00 | 0.00 | 3A4 |
| ATOM | 199 | CA | TYR | 74 | 7.884 | 5.174 | 25.397 | 1.00 | 0.00 | 3A4 |
| ATOM | 200 | CB | TYR | 74 | 6.631 | 5.032 | 26.304 | 1.00 | 0.00 | 3A4 |
| ATOM | 201 | CG | TYR | 74 | 5.298 | 5.291 | 25.625 | 1.00 | 0.00 | 3A4 |
| ATOM | 202 | CD1 | TYR | 74 | 4.515 | 4.223 | 25.146 | 1.00 | 0.00 | 3A4 |
| ATOM | 203 | CD2 | TYR | 74 | 4.797 | 6.603 | 25.491 | 1.00 | 0.00 | 3A4 |
| ATOM | 204 | CE1 | TYR | 74 | 3.279 | 4.456 | 24.524 | 1.00 | 0.00 | 3A4 |
| ATOM | 205 | CE2 | TYR | 74 | 3.564 | 6.844 | 24.870 | 1.00 | 0.00 | 3A4 |
| ATOM | 206 | CZ | TYR | 74 | 2.804 | 5.769 | 24.384 | 1.00 | 0.00 | 3A4 |
| ATOM | 207 | OH | TYR | 74 | 1.560 | 6.006 | 23.757 | 1.00 | 0.00 | 3A4 |
| ATOM | 208 | C | TYR | 74 | 8.100 | 4.007 | 24.486 | 1.00 | 0.00 | 3A4 |
| ATOM | 209 | O | TYR | 74 | 7.256 | 3.682 | 23.658 | 1.00 | 0.00 | 3A4 |
| ATOM | 210 | N | ASP | 75 | 9.272 | 3.357 | 24.603 | 1.00 | 0.00 | 3A4 |
| ATOM | 211 | CA | ASP | 75 | 9.664 | 2.238 | 23.778 | 1.00 | 0.00 | 3A4 |
| ATOM | 212 | CB | ASP | 75 | 11.110 | 2.479 | 23.220 | 1.00 | 0.00 | 3A4 |
| ATOM | 213 | CG | ASP | 75 | 11.483 | 1.556 | 22.044 | 1.00 | 0.00 | 3A4 |
| ATOM | 214 | OD1 | ASP | 75 | 10.755 | 1.577 | 21.015 | 1.00 | 0.00 | 3A4 |
| ATOM | 215 | OD2 | ASP | 75 | 12.500 | 0.822 | 22.166 | 1.00 | 0.00 | 3A4 |
| ATOM | 216 | C | ASP | 75 | 9.580 | 1.007 | 24.657 | 1.00 | 0.00 | 3A4 |
| ATOM | 217 | O | ASP | 75 | 9.275 | 1.091 | 25.845 | 1.00 | 0.00 | 3A4 |
| ATOM | 218 | N | GLY | 76 | 9.925 | −0.191 | 24.123 | 1.00 | 0.00 | 3A4 |
| ATOM | 219 | CA | GLY | 76 | 10.101 | −1.403 | 24.908 | 1.00 | 0.00 | 3A4 |
| ATOM | 220 | C | GLY | 76 | 11.420 | −1.335 | 25.643 | 1.00 | 0.00 | 3A4 |
| ATOM | 221 | O | GLY | 76 | 12.463 | −1.562 | 25.039 | 1.00 | 0.00 | 3A4 |
| ATOM | 222 | N | GLN | 77 | 11.349 | −0.928 | 26.946 | 1.00 | 0.00 | 3A4 |
| ATOM | 223 | CA | GLN | 77 | 12.393 | −0.521 | 27.882 | 1.00 | 0.00 | 3A4 |
| ATOM | 224 | CB | GLN | 77 | 13.760 | −1.300 | 27.835 | 1.00 | 0.00 | 3A4 |
| ATOM | 225 | CG | GLN | 77 | 14.891 | −0.783 | 26.903 | 1.00 | 0.00 | 3A4 |
| ATOM | 226 | CD | GLN | 77 | 15.977 | −1.843 | 26.760 | 1.00 | 0.00 | 3A4 |
| ATOM | 227 | OE1 | GLN | 77 | 16.121 | −2.718 | 27.611 | 1.00 | 0.00 | 3A4 |
| ATOM | 228 | NE2 | GLN | 77 | 16.759 | −1.765 | 25.648 | 1.00 | 0.00 | 3A4 |
| ATOM | 229 | C | GLN | 77 | 12.593 | 0.983 | 27.782 | 1.00 | 0.00 | 3A4 |
| ATOM | 230 | O | GLN | 77 | 12.935 | 1.504 | 26.719 | 1.00 | 0.00 | 3A4 |
| ATOM | 231 | N | GLN | 78 | 12.365 | 1.707 | 28.918 | 1.00 | 0.00 | 3A4 |
| ATOM | 232 | CA | GLN | 78 | 12.698 | 3.105 | 29.188 | 1.00 | 0.00 | 3A4 |
| ATOM | 233 | CB | GLN | 78 | 14.220 | 3.411 | 28.944 | 1.00 | 0.00 | 3A4 |
| ATOM | 234 | CG | GLN | 78 | 14.774 | 4.789 | 29.381 | 1.00 | 0.00 | 3A4 |
| ATOM | 235 | CD | GLN | 78 | 14.622 | 5.043 | 30.889 | 1.00 | 0.00 | 3A4 |
| ATOM | 236 | OE1 | GLN | 78 | 14.409 | 4.138 | 31.694 | 1.00 | 0.00 | 3A4 |
| ATOM | 237 | NE2 | GLN | 78 | 14.757 | 6.336 | 31.296 | 1.00 | 0.00 | 3A4 |
| ATOM | 238 | C | GLN | 78 | 11.820 | 4.135 | 28.453 | 1.00 | 0.00 | 3A4 |
| ATOM | 239 | O | GLN | 78 | 11.794 | 4.125 | 27.222 | 1.00 | 0.00 | 3A4 |
| ATOM | 240 | N | PRO | 79 | 11.120 | 5.089 | 29.129 | 1.00 | 0.00 | 3A4 |
| ATOM | 241 | CA | PRO | 79 | 10.604 | 6.330 | 28.550 | 1.00 | 0.00 | 3A4 |
| ATOM | 242 | CD | PRO | 79 | 10.730 | 4.946 | 30.533 | 1.00 | 0.00 | 3A4 |
| ATOM | 243 | CB | PRO | 79 | 9.535 | 6.798 | 29.557 | 1.00 | 0.00 | 3A4 |
| ATOM | 244 | CG | PRO | 79 | 10.001 | 6.244 | 30.911 | 1.00 | 0.00 | 3A4 |
| ATOM | 245 | C | PRO | 79 | 11.734 | 7.327 | 28.383 | 1.00 | 0.00 | 3A4 |
| ATOM | 246 | O | PRO | 79 | 12.582 | 7.447 | 29.260 | 1.00 | 0.00 | 3A4 |
| ATOM | 247 | N | VAL | 80 | 11.782 | 8.012 | 27.228 | 1.00 | 0.00 | 3A4 |
| ATOM | 248 | CA | VAL | 80 | 12.870 | 8.876 | 26.835 | 1.00 | 0.00 | 3A4 |
| ATOM | 249 | CB | VAL | 80 | 13.698 | 8.283 | 25.679 | 1.00 | 0.00 | 3A4 |
| ATOM | 250 | CG1 | VAL | 80 | 14.441 | 7.026 | 26.173 | 1.00 | 0.00 | 3A4 |
| ATOM | 251 | CG2 | VAL | 80 | 12.828 | 7.928 | 24.440 | 1.00 | 0.00 | 3A4 |
| ATOM | 252 | C | VAL | 80 | 12.250 | 10.173 | 26.392 | 1.00 | 0.00 | 3A4 |
| ATOM | 253 | O | VAL | 80 | 11.117 | 10.204 | 25.939 | 1.00 | 0.00 | 3A4 |
| ATOM | 254 | N | LEU | 81 | 13.003 | 11.285 | 26.466 | 1.00 | 0.00 | 3A4 |
| ATOM | 255 | CA | LEU | 81 | 12.603 | 12.575 | 25.948 | 1.00 | 0.00 | 3A4 |
| ATOM | 256 | CB | LEU | 81 | 12.958 | 13.700 | 26.945 | 1.00 | 0.00 | 3A4 |
| ATOM | 257 | CG | LEU | 81 | 12.302 | 15.079 | 26.699 | 1.00 | 0.00 | 3A4 |
| ATOM | 258 | CD1 | LEU | 81 | 10.822 | 15.078 | 27.116 | 1.00 | 0.00 | 3A4 |
| ATOM | 259 | CD2 | LEU | 81 | 13.057 | 16.197 | 27.437 | 1.00 | 0.00 | 3A4 |
| ATOM | 260 | C | LEU | 81 | 13.352 | 12.751 | 24.647 | 1.00 | 0.00 | 3A4 |
| ATOM | 261 | O | LEU | 81 | 14.529 | 12.427 | 24.569 | 1.00 | 0.00 | 3A4 |
| ATOM | 262 | N | ALA | 82 | 12.695 | 13.249 | 23.585 | 1.00 | 0.00 | 3A4 |
| ATOM | 263 | CA | ALA | 82 | 13.260 | 13.415 | 22.267 | 1.00 | 0.00 | 3A4 |
| ATOM | 264 | CB | ALA | 82 | 12.433 | 12.689 | 21.183 | 1.00 | 0.00 | 3A4 |
| ATOM | 265 | C | ALA | 82 | 13.288 | 14.890 | 21.990 | 1.00 | 0.00 | 3A4 |
| ATOM | 266 | O | ALA | 82 | 12.251 | 15.546 | 22.002 | 1.00 | 0.00 | 3A4 |
| ATOM | 267 | N | ILE | 83 | 14.496 | 15.436 | 21.733 | 1.00 | 0.00 | 3A4 |
| ATOM | 268 | CA | ILE | 83 | 14.742 | 16.837 | 21.486 | 1.00 | 0.00 | 3A4 |
| ATOM | 269 | CB | ILE | 83 | 16.032 | 17.330 | 22.124 | 1.00 | 0.00 | 3A4 |
| ATOM | 270 | CG2 | ILE | 83 | 15.944 | 18.846 | 22.217 | 1.00 | 0.00 | 3A4 |
| ATOM | 271 | CG1 | ILE | 83 | 16.359 | 16.727 | 23.506 | 1.00 | 0.00 | 3A4 |
| ATOM | 272 | CD | ILE | 83 | 15.380 | 17.044 | 24.634 | 1.00 | 0.00 | 3A4 |
| ATOM | 273 | C | ILE | 83 | 14.831 | 17.000 | 19.986 | 1.00 | 0.00 | 3A4 |
| ATOM | 274 | O | ILE | 83 | 15.706 | 16.418 | 19.358 | 1.00 | 0.00 | 3A4 |
| ATOM | 275 | N | THR | 84 | 13.915 | 17.773 | 19.367 | 1.00 | 0.00 | 3A4 |
| ATOM | 276 | CA | THR | 84 | 13.796 | 17.851 | 17.920 | 1.00 | 0.00 | 3A4 |
| ATOM | 277 | CB | THR | 84 | 12.384 | 17.503 | 17.457 | 1.00 | 0.00 | 3A4 |
| ATOM | 278 | OG1 | THR | 84 | 11.371 | 18.330 | 18.025 | 1.00 | 0.00 | 3A4 |
| ATOM | 279 | CG2 | THR | 84 | 12.085 | 16.027 | 17.808 | 1.00 | 0.00 | 3A4 |
| ATOM | 280 | C | THR | 84 | 14.259 | 19.175 | 17.344 | 1.00 | 0.00 | 3A4 |
| ATOM | 281 | O | THR | 84 | 14.500 | 19.282 | 16.144 | 1.00 | 0.00 | 3A4 |
| ATOM | 282 | N | ASP | 85 | 14.371 | 20.231 | 18.176 | 1.00 | 0.00 | 3A4 |
| ATOM | 283 | CA | ASP | 85 | 14.595 | 21.594 | 17.732 | 1.00 | 0.00 | 3A4 |
| ATOM | 284 | CB | ASP | 85 | 13.703 | 22.550 | 18.577 | 1.00 | 0.00 | 3A4 |
| ATOM | 285 | CG | ASP | 85 | 13.371 | 23.873 | 17.872 | 1.00 | 0.00 | 3A4 |
| ATOM | 286 | OD1 | ASP | 85 | 12.666 | 23.828 | 16.828 | 1.00 | 0.00 | 3A4 |
| ATOM | 287 | OD2 | ASP | 85 | 13.808 | 24.942 | 18.373 | 1.00 | 0.00 | 3A4 |
| ATOM | 288 | C | ASP | 85 | 16.050 | 21.928 | 17.888 | 1.00 | 0.00 | 3A4 |
| ATOM | 289 | O | ASP | 85 | 16.660 | 21.452 | 18.843 | 1.00 | 0.00 | 3A4 |
| ATOM | 290 | N | PRO | 86 | 16.683 | 22.732 | 17.024 | 1.00 | 0.00 | 3A4 |
| ATOM | 291 | CA | PRO | 86 | 18.101 | 23.035 | 17.076 | 1.00 | 0.00 | 3A4 |
| ATOM | 292 | CD | PRO | 86 | 16.102 | 23.257 | 15.807 | 1.00 | 0.00 | 3A4 |
| ATOM | 293 | CB | PRO | 86 | 18.421 | 23.751 | 15.747 | 1.00 | 0.00 | 3A4 |
| ATOM | 294 | CG | PRO | 86 | 17.079 | 24.309 | 15.294 | 1.00 | 0.00 | 3A4 |
| ATOM | 295 | C | PRO | 86 | 18.425 | 23.863 | 18.284 | 1.00 | 0.00 | 3A4 |
| ATOM | 296 | O | PRO | 86 | 19.466 | 23.647 | 18.892 | 1.00 | 0.00 | 3A4 |
| ATOM | 297 | N | ASP | 87 | 17.511 | 24.756 | 18.711 | 1.00 | 0.00 | 3A4 |
| ATOM | 298 | CA | ASP | 87 | 17.636 | 25.559 | 19.902 | 1.00 | 0.00 | 3A4 |
| ATOM | 299 | CB | ASP | 87 | 16.417 | 26.494 | 20.029 | 1.00 | 0.00 | 3A4 |
| ATOM | 300 | CG | ASP | 87 | 16.305 | 27.442 | 18.820 | 1.00 | 0.00 | 3A4 |
| ATOM | 301 | OD1 | ASP | 87 | 17.357 | 27.851 | 18.258 | 1.00 | 0.00 | 3A4 |
| ATOM | 302 | OD2 | ASP | 87 | 15.148 | 27.764 | 18.439 | 1.00 | 0.00 | 3A4 |
| ATOM | 303 | C | ASP | 87 | 17.724 | 24.745 | 21.171 | 1.00 | 0.00 | 3A4 |
| ATOM | 304 | O | ASP | 87 | 18.546 | 25.018 | 22.032 | 1.00 | 0.00 | 3A4 |
| ATOM | 305 | N | MET | 88 | 16.902 | 23.678 | 21.253 | 1.00 | 0.00 | 3A4 |
| ATOM | 306 | CA | MET | 88 | 16.847 | 22.776 | 22.375 | 1.00 | 0.00 | 3A4 |
| ATOM | 307 | CB | MET | 88 | 15.536 | 22.000 | 22.396 | 1.00 | 0.00 | 3A4 |
| ATOM | 308 | CG | MET | 88 | 14.362 | 22.948 | 22.644 | 1.00 | 0.00 | 3A4 |
| ATOM | 309 | SD | MET | 88 | 12.947 | 22.177 | 23.469 | 1.00 | 0.00 | 3A4 |
| ATOM | 310 | CE | MET | 88 | 13.722 | 21.914 | 25.087 | 1.00 | 0.00 | 3A4 |
| ATOM | 311 | C | MET | 88 | 18.007 | 21.812 | 22.436 | 1.00 | 0.00 | 3A4 |
| ATOM | 312 | O | MET | 88 | 18.524 | 21.506 | 23.506 | 1.00 | 0.00 | 3A4 |
| ATOM | 313 | N | ILE | 89 | 18.500 | 21.349 | 21.264 | 1.00 | 0.00 | 3A4 |
| ATOM | 314 | CA | ILE | 89 | 19.637 | 20.450 | 21.170 | 1.00 | 0.00 | 3A4 |
| ATOM | 315 | CB | ILE | 89 | 19.679 | 19.800 | 19.793 | 1.00 | 0.00 | 3A4 |
| ATOM | 316 | CG2 | ILE | 89 | 21.088 | 19.217 | 19.442 | 1.00 | 0.00 | 3A4 |
| ATOM | 317 | CG1 | ILE | 89 | 18.596 | 18.681 | 19.910 | 1.00 | 0.00 | 3A4 |
| ATOM | 318 | CD | ILE | 89 | 18.390 | 17.678 | 18.773 | 1.00 | 0.00 | 3A4 |
| ATOM | 319 | C | ILE | 89 | 20.916 | 21.161 | 21.538 | 1.00 | 0.00 | 3A4 |
| ATOM | 320 | O | ILE | 89 | 21.722 | 20.655 | 22.310 | 1.00 | 0.00 | 3A4 |
| ATOM | 321 | N | LYS | 90 | 21.096 | 22.421 | 21.089 | 1.00 | 0.00 | 3A4 |
| ATOM | 322 | CA | LYS | 90 | 22.214 | 23.250 | 21.481 | 1.00 | 0.00 | 3A4 |
| ATOM | 323 | CB | LYS | 90 | 22.225 | 24.577 | 20.719 | 1.00 | 0.00 | 3A4 |
| ATOM | 324 | CG | LYS | 90 | 23.531 | 25.387 | 20.790 | 1.00 | 0.00 | 3A4 |
| ATOM | 325 | CD | LYS | 90 | 23.546 | 26.569 | 19.811 | 1.00 | 0.00 | 3A4 |
| ATOM | 326 | CE | LYS | 90 | 24.875 | 27.339 | 19.774 | 1.00 | 0.00 | 3A4 |
| ATOM | 327 | NZ | LYS | 90 | 25.167 | 27.975 | 21.082 | 1.00 | 0.00 | 3A4 |
| ATOM | 328 | C | LYS | 90 | 22.221 | 23.564 | 22.961 | 1.00 | 0.00 | 3A4 |
| ATOM | 329 | O | LYS | 90 | 23.248 | 23.567 | 23.612 | 1.00 | 0.00 | 3A4 |
| ATOM | 330 | N | THR | 91 | 21.031 | 23.752 | 23.561 | 1.00 | 0.00 | 3A4 |
| ATOM | 331 | CA | THR | 91 | 20.855 | 23.981 | 24.981 | 1.00 | 0.00 | 3A4 |
| ATOM | 332 | CB | THR | 91 | 19.434 | 24.380 | 25.293 | 1.00 | 0.00 | 3A4 |
| ATOM | 333 | OG1 | THR | 91 | 19.170 | 25.637 | 24.681 | 1.00 | 0.00 | 3A4 |
| ATOM | 334 | CG2 | THR | 91 | 19.109 | 24.554 | 26.810 | 1.00 | 0.00 | 3A4 |
| ATOM | 335 | C | THR | 91 | 21.198 | 22.782 | 25.817 | 1.00 | 0.00 | 3A4 |
| ATOM | 336 | O | THR | 91 | 21.850 | 22.889 | 26.851 | 1.00 | 0.00 | 3A4 |
| ATOM | 337 | N | VAL | 92 | 20.849 | 21.578 | 25.307 | 1.00 | 0.00 | 3A4 |
| ATOM | 338 | CA | VAL | 92 | 21.125 | 20.315 | 25.945 | 1.00 | 0.00 | 3A4 |
| ATOM | 339 | CB | VAL | 92 | 20.299 | 19.178 | 25.353 | 1.00 | 0.00 | 3A4 |
| ATOM | 340 | CG1 | VAL | 92 | 21.025 | 18.247 | 24.370 | 1.00 | 0.00 | 3A4 |
| ATOM | 341 | CG2 | VAL | 92 | 19.643 | 18.370 | 26.498 | 1.00 | 0.00 | 3A4 |
| ATOM | 342 | C | VAL | 92 | 22.600 | 20.047 | 26.089 | 1.00 | 0.00 | 3A4 |
| ATOM | 343 | O | VAL | 92 | 22.991 | 19.412 | 27.041 | 1.00 | 0.00 | 3A4 |
| ATOM | 344 | N | LEU | 93 | 23.483 | 20.706 | 25.311 | 1.00 | 0.00 | 3A4 |
| ATOM | 345 | CA | LEU | 93 | 24.914 | 20.851 | 25.543 | 1.00 | 0.00 | 3A4 |
| ATOM | 346 | CB | LEU | 93 | 25.432 | 21.742 | 24.407 | 1.00 | 0.00 | 3A4 |
| ATOM | 347 | CG | LEU | 93 | 26.456 | 21.108 | 23.525 | 1.00 | 0.00 | 3A4 |
| ATOM | 348 | CD1 | LEU | 93 | 25.796 | 19.892 | 22.820 | 1.00 | 0.00 | 3A4 |
| ATOM | 349 | CD2 | LEU | 93 | 26.955 | 22.238 | 22.601 | 1.00 | 0.00 | 3A4 |
| ATOM | 350 | C | LEU | 93 | 25.419 | 21.473 | 26.847 | 1.00 | 0.00 | 3A4 |
| ATOM | 351 | O | LEU | 93 | 24.654 | 21.997 | 27.655 | 1.00 | 0.00 | 3A4 |
| ATOM | 352 | N | VAL | 94 | 26.774 | 21.385 | 27.035 | 1.00 | 0.00 | 3A4 |
| ATOM | 353 | CA | VAL | 94 | 27.599 | 21.729 | 28.196 | 1.00 | 0.00 | 3A4 |
| ATOM | 354 | CB | VAL | 94 | 27.328 | 23.082 | 28.879 | 1.00 | 0.00 | 3A4 |
| ATOM | 355 | CG1 | VAL | 94 | 28.368 | 23.358 | 30.001 | 1.00 | 0.00 | 3A4 |
| ATOM | 356 | CG2 | VAL | 94 | 27.427 | 24.201 | 27.813 | 1.00 | 0.00 | 3A4 |
| ATOM | 357 | C | VAL | 94 | 27.515 | 20.540 | 29.137 | 1.00 | 0.00 | 3A4 |
| ATOM | 358 | O | VAL | 94 | 26.875 | 20.578 | 30.188 | 1.00 | 0.00 | 3A4 |
| ATOM | 359 | N | LYS | 95 | 28.141 | 19.421 | 28.690 | 1.00 | 0.00 | 3A4 |
| ATOM | 360 | CA | LYS | 95 | 28.019 | 18.101 | 29.261 | 1.00 | 0.00 | 3A4 |
| ATOM | 361 | CB | LYS | 95 | 27.404 | 17.078 | 28.259 | 1.00 | 0.00 | 3A4 |
| ATOM | 362 | CG | LYS | 95 | 27.757 | 17.284 | 26.778 | 1.00 | 0.00 | 3A4 |
| ATOM | 363 | CD | LYS | 95 | 27.133 | 16.199 | 25.898 | 1.00 | 0.00 | 3A4 |
| ATOM | 364 | CE | LYS | 95 | 27.017 | 16.559 | 24.413 | 1.00 | 0.00 | 3A4 |
| ATOM | 365 | NZ | LYS | 95 | 28.338 | 16.819 | 23.809 | 1.00 | 0.00 | 3A4 |
| ATOM | 366 | C | LYS | 95 | 29.379 | 17.675 | 29.736 | 1.00 | 0.00 | 3A4 |
| ATOM | 367 | O | LYS | 95 | 30.123 | 16.990 | 29.040 | 1.00 | 0.00 | 3A4 |
| ATOM | 368 | N | GLU | 96 | 29.703 | 18.085 | 30.988 | 1.00 | 0.00 | 3A4 |
| ATOM | 369 | CA | GLU | 96 | 30.926 | 17.783 | 31.698 | 1.00 | 0.00 | 3A4 |
| ATOM | 370 | CB | GLU | 96 | 31.795 | 19.051 | 31.950 | 1.00 | 0.00 | 3A4 |
| ATOM | 371 | CG | GLU | 96 | 31.039 | 20.290 | 32.486 | 1.00 | 0.00 | 3A4 |
| ATOM | 372 | CD | GLU | 96 | 32.012 | 21.467 | 32.599 | 1.00 | 0.00 | 3A4 |
| ATOM | 373 | OE1 | GLU | 96 | 31.790 | 22.493 | 31.901 | 1.00 | 0.00 | 3A4 |
| ATOM | 374 | OE2 | GLU | 96 | 32.990 | 21.357 | 33.388 | 1.00 | 0.00 | 3A4 |
| ATOM | 375 | C | GLU | 96 | 30.517 | 17.078 | 32.969 | 1.00 | 0.00 | 3A4 |
| ATOM | 376 | O | GLU | 96 | 30.732 | 17.576 | 34.073 | 1.00 | 0.00 | 3A4 |
| ATOM | 377 | N | CYS | 97 | 29.924 | 15.869 | 32.763 | 1.00 | 0.00 | 3A4 |
| ATOM | 378 | CA | CYS | 97 | 29.436 | 14.865 | 33.693 | 1.00 | 0.00 | 3A4 |
| ATOM | 379 | CB | CYS | 97 | 29.558 | 15.141 | 35.235 | 1.00 | 0.00 | 3A4 |
| ATOM | 380 | SG | CYS | 97 | 29.186 | 13.706 | 36.313 | 1.00 | 0.00 | 3A4 |
| ATOM | 381 | C | CYS | 97 | 28.000 | 14.633 | 33.277 | 1.00 | 0.00 | 3A4 |
| ATOM | 382 | O | CYS | 97 | 27.076 | 15.253 | 33.800 | 1.00 | 0.00 | 3A4 |
| ATOM | 383 | N | TYR | 98 | 27.817 | 13.703 | 32.305 | 1.00 | 0.00 | 3A4 |
| ATOM | 384 | CA | TYR | 98 | 26.556 | 13.216 | 31.792 | 1.00 | 0.00 | 3A4 |
| ATOM | 385 | CB | TYR | 98 | 26.197 | 13.814 | 30.380 | 1.00 | 0.00 | 3A4 |
| ATOM | 386 | CG | TYR | 98 | 25.205 | 14.939 | 30.543 | 1.00 | 0.00 | 3A4 |
| ATOM | 387 | CD1 | TYR | 98 | 25.573 | 16.198 | 31.050 | 1.00 | 0.00 | 3A4 |
| ATOM | 388 | CD2 | TYR | 98 | 23.862 | 14.731 | 30.197 | 1.00 | 0.00 | 3A4 |
| ATOM | 389 | CE1 | TYR | 98 | 24.626 | 17.224 | 31.198 | 1.00 | 0.00 | 3A4 |
| ATOM | 390 | CE2 | TYR | 98 | 22.901 | 15.740 | 30.351 | 1.00 | 0.00 | 3A4 |
| ATOM | 391 | CZ | TYR | 98 | 23.286 | 16.993 | 30.849 | 1.00 | 0.00 | 3A4 |
| ATOM | 392 | OH | TYR | 98 | 22.323 | 18.015 | 30.999 | 1.00 | 0.00 | 3A4 |
| ATOM | 393 | C | TYR | 98 | 26.800 | 11.735 | 31.683 | 1.00 | 0.00 | 3A4 |
| ATOM | 394 | O | TYR | 98 | 27.629 | 11.292 | 30.891 | 1.00 | 0.00 | 3A4 |
| ATOM | 395 | N | SER | 99 | 26.097 | 10.928 | 32.531 | 1.00 | 0.00 | 3A4 |
| ATOM | 396 | CA | SER | 99 | 26.414 | 9.536 | 32.838 | 1.00 | 0.00 | 3A4 |
| ATOM | 397 | CB | SER | 99 | 25.870 | 9.141 | 34.247 | 1.00 | 0.00 | 3A4 |
| ATOM | 398 | OG | SER | 99 | 26.515 | 7.992 | 34.791 | 1.00 | 0.00 | 3A4 |
| ATOM | 399 | C | SER | 99 | 25.931 | 8.568 | 31.771 | 1.00 | 0.00 | 3A4 |
| ATOM | 400 | O | SER | 99 | 24.885 | 8.764 | 31.156 | 1.00 | 0.00 | 3A4 |
| ATOM | 401 | N | VAL | 100 | 26.759 | 7.507 | 31.524 | 1.00 | 0.00 | 3A4 |
| ATOM | 402 | CA | VAL | 100 | 26.647 | 6.426 | 30.542 | 1.00 | 0.00 | 3A4 |
| ATOM | 403 | CB | VAL | 100 | 25.344 | 5.612 | 30.576 | 1.00 | 0.00 | 3A4 |
| ATOM | 404 | CG1 | VAL | 100 | 25.483 | 4.356 | 29.677 | 1.00 | 0.00 | 3A4 |
| ATOM | 405 | CG2 | VAL | 100 | 25.062 | 5.167 | 32.031 | 1.00 | 0.00 | 3A4 |
| ATOM | 406 | C | VAL | 100 | 26.977 | 6.962 | 29.152 | 1.00 | 0.00 | 3A4 |
| ATOM | 407 | O | VAL | 100 | 28.088 | 6.758 | 28.673 | 1.00 | 0.00 | 3A4 |
| ATOM | 408 | N | PHE | 101 | 26.002 | 7.682 | 28.517 | 1.00 | 0.00 | 3A4 |
| ATOM | 409 | CA | PHE | 101 | 26.131 | 8.581 | 27.372 | 1.00 | 0.00 | 3A4 |
| ATOM | 410 | CB | PHE | 101 | 27.487 | 9.383 | 27.359 | 1.00 | 0.00 | 3A4 |
| ATOM | 411 | CG | PHE | 101 | 27.503 | 10.546 | 26.402 | 1.00 | 0.00 | 3A4 |
| ATOM | 412 | CD1 | PHE | 101 | 26.587 | 11.609 | 26.522 | 1.00 | 0.00 | 3A4 |
| ATOM | 413 | CD2 | PHE | 101 | 28.432 | 10.563 | 25.350 | 1.00 | 0.00 | 3A4 |
| ATOM | 414 | CE1 | PHE | 101 | 26.580 | 12.640 | 25.573 | 1.00 | 0.00 | 3A4 |
| ATOM | 415 | CE2 | PHE | 101 | 28.435 | 11.597 | 24.418 | 1.00 | 0.00 | 3A4 |
| ATOM | 416 | CZ | PHE | 101 | 27.492 | 12.629 | 24.516 | 1.00 | 0.00 | 3A4 |
| ATOM | 417 | C | PHE | 101 | 25.860 | 7.863 | 26.049 | 1.00 | 0.00 | 3A4 |
| ATOM | 418 | O | PHE | 101 | 25.613 | 8.506 | 25.030 | 1.00 | 0.00 | 3A4 |
| ATOM | 419 | N | THR | 102 | 25.881 | 6.507 | 26.048 | 1.00 | 0.00 | 3A4 |
| ATOM | 420 | CA | THR | 102 | 25.468 | 5.674 | 24.939 | 1.00 | 0.00 | 3A4 |
| ATOM | 421 | CE | THR | 102 | 26.561 | 5.456 | 23.875 | 1.00 | 0.00 | 3A4 |
| ATOM | 422 | OG1 | THR | 102 | 26.134 | 4.618 | 22.803 | 1.00 | 0.00 | 3A4 |
| ATOM | 423 | CG2 | THR | 102 | 27.920 | 4.971 | 24.441 | 1.00 | 0.00 | 3A4 |
| ATOM | 424 | C | THR | 102 | 24.949 | 4.448 | 25.660 | 1.00 | 0.00 | 3A4 |
| ATOM | 425 | O | THR | 102 | 25.680 | 3.511 | 25.976 | 1.00 | 0.00 | 3A4 |
| ATOM | 426 | N | ASN | 103 | 23.629 | 4.491 | 25.991 | 1.00 | 0.00 | 3A4 |
| ATOM | 427 | CA | ASN | 103 | 22.973 | 3.634 | 26.965 | 1.00 | 0.00 | 3A4 |
| ATOM | 428 | CB | ASN | 103 | 22.145 | 4.482 | 28.000 | 1.00 | 0.00 | 3A4 |
| ATOM | 429 | CG | ASN | 103 | 21.193 | 5.519 | 27.373 | 1.00 | 0.00 | 3A4 |
| ATOM | 430 | OD1 | ASN | 103 | 21.530 | 6.702 | 27.345 | 1.00 | 0.00 | 3A4 |
| ATOM | 431 | ND2 | ASN | 103 | 19.993 | 5.093 | 26.893 | 1.00 | 0.00 | 3A4 |
| ATOM | 432 | C | ASN | 103 | 22.171 | 2.529 | 26.295 | 1.00 | 0.00 | 3A4 |
| ATOM | 433 | O | ASN | 103 | 22.526 | 2.061 | 25.214 | 1.00 | 0.00 | 3A4 |
| ATOM | 434 | N | ARG | 104 | 21.099 | 2.084 | 27.018 | 1.00 | 0.00 | 3A4 |
| ATOM | 435 | CA | ARG | 104 | 20.255 | 0.904 | 26.922 | 1.00 | 0.00 | 3A4 |
| ATOM | 436 | CB | ARG | 104 | 20.057 | 0.305 | 25.521 | 1.00 | 0.00 | 3A4 |
| ATOM | 437 | CG | ARG | 104 | 19.266 | 1.224 | 24.571 | 1.00 | 0.00 | 3A4 |
| ATOM | 438 | CD | ARG | 104 | 19.318 | 0.794 | 23.096 | 1.00 | 0.00 | 3A4 |
| ATOM | 439 | NE | ARG | 104 | 20.722 | 1.001 | 22.589 | 1.00 | 0.00 | 3A4 |
| ATOM | 440 | CZ | ARG | 104 | 21.086 | 0.812 | 21.280 | 1.00 | 0.00 | 3A4 |
| ATOM | 441 | NH1 | ARG | 104 | 22.383 | 1.027 | 20.912 | 1.00 | 0.00 | 3A4 |
| ATOM | 442 | NH2 | ARG | 104 | 20.184 | 0.403 | 20.340 | 1.00 | 0.00 | 3A4 |
| ATOM | 443 | C | ARG | 104 | 20.801 | −0.116 | 27.892 | 1.00 | 0.00 | 3A4 |
| ATOM | 444 | O | ARG | 104 | 21.996 | −0.173 | 28.177 | 1.00 | 0.00 | 3A4 |
| ATOM | 445 | N | ARG | 105 | 19.899 | −0.953 | 28.439 | 1.00 | 0.00 | 3A4 |
| ATOM | 446 | CA | ARG | 105 | 20.180 | −1.924 | 29.470 | 1.00 | 0.00 | 3A4 |
| ATOM | 447 | CB | ARG | 105 | 18.917 | −2.306 | 30.269 | 1.00 | 0.00 | 3A4 |
| ATOM | 448 | CG | ARG | 105 | 18.288 | −1.109 | 31.012 | 1.00 | 0.00 | 3A4 |
| ATOM | 449 | CD | ARG | 105 | 17.273 | −0.267 | 30.201 | 1.00 | 0.00 | 3A4 |
| ATOM | 450 | NE | ARG | 105 | 16.999 | 1.019 | 30.940 | 1.00 | 0.00 | 3A4 |
| ATOM | 451 | CZ | ARG | 105 | 17.736 | 2.168 | 30.769 | 1.00 | 0.00 | 3A4 |
| ATOM | 452 | NH1 | ARG | 105 | 17.471 | 3.253 | 31.550 | 1.00 | 0.00 | 3A4 |
| ATOM | 453 | NH2 | ARG | 105 | 18.734 | 2.262 | 29.841 | 1.00 | 0.00 | 3A4 |
| ATOM | 454 | C | ARG | 105 | 20.851 | −3.183 | 28.984 | 1.00 | 0.00 | 3A4 |
| ATOM | 455 | O | ARG | 105 | 21.862 | −3.545 | 29.576 | 1.00 | 0.00 | 3A4 |
| ATOM | 456 | N | PRO | 106 | 20.439 | −3.879 | 27.908 | 1.00 | 0.00 | 3A4 |
| ATOM | 457 | CA | PRO | 106 | 21.118 | −5.066 | 27.409 | 1.00 | 0.00 | 3A4 |
| ATOM | 458 | CD | PRO | 106 | 19.166 | −3.707 | 27.212 | 1.00 | 0.00 | 3A4 |
| ATOM | 459 | CB | PRO | 106 | 20.292 | −5.535 | 26.208 | 1.00 | 0.00 | 3A4 |
| ATOM | 460 | CG | PRO | 106 | 19.354 | −4.383 | 25.861 | 1.00 | 0.00 | 3A4 |
| ATOM | 461 | C | PRO | 106 | 22.531 | −4.785 | 26.960 | 1.00 | 0.00 | 3A4 |
| ATOM | 462 | O | PRO | 106 | 23.412 | −5.580 | 27.239 | 1.00 | 0.00 | 3A4 |
| ATOM | 463 | N | PHE | 107 | 22.789 | −3.612 | 26.347 | 1.00 | 0.00 | 3A4 |
| ATOM | 464 | CA | PHE | 107 | 24.092 | −3.149 | 25.939 | 1.00 | 0.00 | 3A4 |
| ATOM | 465 | CB | PHE | 107 | 23.996 | −1.854 | 25.067 | 1.00 | 0.00 | 3A4 |
| ATOM | 466 | CG | PHE | 107 | 23.377 | −2.185 | 23.730 | 1.00 | 0.00 | 3A4 |
| ATOM | 467 | CD1 | PHE | 107 | 24.194 | −2.404 | 22.604 | 1.00 | 0.00 | 3A4 |
| ATOM | 468 | CD2 | PHE | 107 | 21.982 | −2.301 | 23.578 | 1.00 | 0.00 | 3A4 |
| ATOM | 469 | CE1 | PHE | 107 | 23.633 | −2.730 | 21.360 | 1.00 | 0.00 | 3A4 |
| ATOM | 470 | CE2 | PHE | 107 | 21.415 | −2.640 | 22.342 | 1.00 | 0.00 | 3A4 |
| ATOM | 471 | CZ | PHE | 107 | 22.243 | −2.850 | 21.231 | 1.00 | 0.00 | 3A4 |
| ATOM | 472 | C | PHE | 107 | 24.996 | −2.874 | 27.117 | 1.00 | 0.00 | 3A4 |
| ATOM | 473 | O | PHE | 107 | 26.183 | −3.178 | 27.071 | 1.00 | 0.00 | 3A4 |
| ATOM | 474 | N | GLY | 108 | 24.445 | −2.352 | 28.235 | 1.00 | 0.00 | 3A4 |
| ATOM | 475 | CA | GLY | 108 | 25.169 | −2.115 | 29.465 | 1.00 | 0.00 | 3A4 |
| ATOM | 476 | C | GLY | 108 | 25.703 | −3.372 | 30.122 | 1.00 | 0.00 | 3A4 |
| ATOM | 477 | O | GLY | 108 | 26.825 | −3.365 | 30.623 | 1.00 | 0.00 | 3A4 |
| ATOM | 478 | N | PRO | 109 | 24.957 | −4.476 | 30.112 | 1.00 | 0.00 | 3A4 |
| ATOM | 479 | CA | PRO | 109 | 25.476 | −5.825 | 30.348 | 1.00 | 0.00 | 3A4 |
| ATOM | 480 | CD | PRO | 109 | 24.065 | −4.305 | 31.286 | 1.00 | 0.00 | 3A4 |
| ATOM | 481 | CB | PRO | 109 | 24.191 | −6.637 | 30.615 | 1.00 | 0.00 | 3A4 |
| ATOM | 482 | CG | PRO | 109 | 23.330 | −5.658 | 31.433 | 1.00 | 0.00 | 3A4 |
| ATOM | 483 | C | PRO | 109 | 26.330 | −6.541 | 29.318 | 1.00 | 0.00 | 3A4 |
| ATOM | 484 | O | PRO | 109 | 27.456 | −6.908 | 29.658 | 1.00 | 0.00 | 3A4 |
| ATOM | 485 | N | VAL | 110 | 25.774 | −6.864 | 28.119 | 1.00 | 0.00 | 3A4 |
| ATOM | 486 | CA | VAL | 110 | 26.331 | −7.778 | 27.133 | 1.00 | 0.00 | 3A4 |
| ATOM | 487 | CB | VAL | 110 | 25.389 | −8.936 | 26.768 | 1.00 | 0.00 | 3A4 |
| ATOM | 488 | CG1 | VAL | 110 | 25.412 | −9.943 | 27.938 | 1.00 | 0.00 | 3A4 |
| ATOM | 489 | CG2 | VAL | 110 | 23.943 | −8.492 | 26.450 | 1.00 | 0.00 | 3A4 |
| ATOM | 490 | C | VAL | 110 | 26.744 | −6.971 | 25.923 | 1.00 | 0.00 | 3A4 |
| ATOM | 491 | O | VAL | 110 | 25.981 | −6.165 | 25.392 | 1.00 | 0.00 | 3A4 |
| ATOM | 492 | N | GLY | 111 | 28.021 | −7.174 | 25.497 | 1.00 | 0.00 | 3A4 |
| ATOM | 493 | CA | GLY | 111 | 28.732 | −6.403 | 24.496 | 1.00 | 0.00 | 3A4 |
| ATOM | 494 | C | GLY | 111 | 29.773 | −5.622 | 25.245 | 1.00 | 0.00 | 3A4 |
| ATOM | 495 | O | GLY | 111 | 29.468 | −4.588 | 25.839 | 1.00 | 0.00 | 3A4 |
| ATOM | 496 | N | PHE | 112 | 31.035 | −6.136 | 25.266 | 1.00 | 0.00 | 3A4 |
| ATOM | 497 | CA | PHE | 112 | 32.105 | −5.711 | 26.158 | 1.00 | 0.00 | 3A4 |
| ATOM | 498 | CB | PHE | 112 | 32.907 | −6.929 | 26.720 | 1.00 | 0.00 | 3A4 |
| ATOM | 499 | CG | PHE | 112 | 31.979 | −7.800 | 27.529 | 1.00 | 0.00 | 3A4 |
| ATOM | 500 | CD1 | PHE | 112 | 31.631 | −9.095 | 27.096 | 1.00 | 0.00 | 3A4 |
| ATOM | 501 | CD2 | PHE | 112 | 31.424 | −7.318 | 28.732 | 1.00 | 0.00 | 3A4 |
| ATOM | 502 | CE1 | PHE | 112 | 30.746 | −9.883 | 27.847 | 1.00 | 0.00 | 3A4 |
| ATOM | 503 | CE2 | PHE | 112 | 30.537 | −8.104 | 29.480 | 1.00 | 0.00 | 3A4 |
| ATOM | 504 | CZ | PHE | 112 | 30.195 | −9.387 | 29.035 | 1.00 | 0.00 | 3A4 |
| ATOM | 505 | C | PHE | 112 | 33.050 | −4.749 | 25.472 | 1.00 | 0.00 | 3A4 |
| ATOM | 506 | O | PHE | 112 | 34.190 | −5.078 | 25.148 | 1.00 | 0.00 | 3A4 |
| ATOM | 507 | N | MET | 113 | 32.562 | −3.489 | 25.295 | 1.00 | 0.00 | 3A4 |
| ATOM | 508 | CA | MET | 113 | 33.296 | −2.314 | 24.869 | 1.00 | 0.00 | 3A4 |
| ATOM | 509 | CB | MET | 113 | 32.602 | −1.505 | 23.728 | 1.00 | 0.00 | 3A4 |
| ATOM | 510 | CG | MET | 113 | 32.554 | −2.210 | 22.356 | 1.00 | 0.00 | 3A4 |
| ATOM | 511 | SD | MET | 113 | 31.429 | −3.639 | 22.184 | 1.00 | 0.00 | 3A4 |
| ATOM | 512 | CE | MET | 113 | 29.851 | −2.787 | 22.471 | 1.00 | 0.00 | 3A4 |
| ATOM | 513 | C | MET | 113 | 33.386 | −1.481 | 26.125 | 1.00 | 0.00 | 3A4 |
| ATOM | 514 | O | MET | 113 | 32.371 | −1.027 | 26.652 | 1.00 | 0.00 | 3A4 |
| ATOM | 515 | N | LYS | 114 | 34.630 | −1.321 | 26.660 | 1.00 | 0.00 | 3A4 |
| ATOM | 516 | CA | LYS | 114 | 34.932 | −0.910 | 28.025 | 1.00 | 0.00 | 3A4 |
| ATOM | 517 | CB | LYS | 114 | 36.207 | −1.643 | 28.544 | 1.00 | 0.00 | 3A4 |
| ATOM | 518 | CG | LYS | 114 | 36.129 | −3.170 | 28.392 | 1.00 | 0.00 | 3A4 |
| ATOM | 519 | CD | LYS | 114 | 37.411 | −3.890 | 28.832 | 1.00 | 0.00 | 3A4 |
| ATOM | 520 | CE | LYS | 114 | 37.355 | −5.417 | 28.667 | 1.00 | 0.00 | 3A4 |
| ATOM | 521 | NZ | LYS | 114 | 37.190 | −5.806 | 27.244 | 1.00 | 0.00 | 3A4 |
| ATOM | 522 | C | LYS | 114 | 35.074 | 0.600 | 28.136 | 1.00 | 0.00 | 3A4 |
| ATOM | 523 | O | LYS | 114 | 34.485 | 1.339 | 27.348 | 1.00 | 0.00 | 3A4 |
| ATOM | 524 | N | SER | 115 | 35.857 | 1.092 | 29.141 | 1.00 | 0.00 | 3A4 |
| ATOM | 525 | CA | SER | 115 | 35.955 | 2.486 | 29.566 | 1.00 | 0.00 | 3A4 |
| ATOM | 526 | CB | SER | 115 | 36.419 | 2.597 | 31.048 | 1.00 | 0.00 | 3A4 |
| ATOM | 527 | OG | SER | 115 | 35.505 | 1.921 | 31.901 | 1.00 | 0.00 | 3A4 |
| ATOM | 528 | C | SER | 115 | 36.891 | 3.307 | 28.696 | 1.00 | 0.00 | 3A4 |
| ATOM | 529 | O | SER | 115 | 38.106 | 3.302 | 28.890 | 1.00 | 0.00 | 3A4 |
| ATOM | 530 | N | ALA | 116 | 36.300 | 4.041 | 27.709 | 1.00 | 0.00 | 3A4 |
| ATOM | 531 | CA | ALA | 116 | 36.990 | 4.902 | 26.770 | 1.00 | 0.00 | 3A4 |
| ATOM | 532 | CB | ALA | 116 | 36.806 | 4.440 | 25.323 | 1.00 | 0.00 | 3A4 |
| ATOM | 533 | C | ALA | 116 | 36.445 | 6.277 | 26.942 | 1.00 | 0.00 | 3A4 |
| ATOM | 534 | O | ALA | 116 | 35.934 | 6.634 | 27.995 | 1.00 | 0.00 | 3A4 |
| ATOM | 535 | N | ILE | 117 | 36.498 | 7.116 | 25.886 | 1.00 | 0.00 | 3A4 |
| ATOM | 536 | CA | ILE | 117 | 36.054 | 8.497 | 25.985 | 1.00 | 0.00 | 3A4 |
| ATOM | 537 | CB | ILE | 117 | 36.818 | 9.363 | 24.999 | 1.00 | 0.00 | 3A4 |
| ATOM | 538 | CG2 | ILE | 117 | 36.637 | 8.922 | 23.524 | 1.00 | 0.00 | 3A4 |
| ATOM | 539 | CG1 | ILE | 117 | 36.647 | 10.857 | 25.212 | 1.00 | 0.00 | 3A4 |
| ATOM | 540 | CD | ILE | 117 | 37.122 | 11.262 | 26.574 | 1.00 | 0.00 | 3A4 |
| ATOM | 541 | C | ILE | 117 | 34.556 | 8.659 | 25.822 | 1.00 | 0.00 | 3A4 |
| ATOM | 542 | O | ILE | 117 | 33.924 | 9.474 | 26.492 | 1.00 | 0.00 | 3A4 |
| ATOM | 543 | N | SER | 118 | 33.923 | 7.843 | 24.946 | 1.00 | 0.00 | 3A4 |
| ATOM | 544 | CA | SER | 118 | 32.533 | 8.033 | 24.588 | 1.00 | 0.00 | 3A4 |
| ATOM | 545 | CB | SER | 118 | 32.205 | 7.525 | 23.146 | 1.00 | 0.00 | 3A4 |
| ATOM | 546 | OG | SER | 118 | 32.499 | 6.147 | 22.946 | 1.00 | 0.00 | 3A4 |
| ATOM | 547 | C | SER | 118 | 31.585 | 7.455 | 25.606 | 1.00 | 0.00 | 3A4 |
| ATOM | 548 | O | SER | 118 | 30.395 | 7.686 | 25.524 | 1.00 | 0.00 | 3A4 |
| ATOM | 549 | N | ILE | 119 | 32.084 | 6.735 | 26.630 | 1.00 | 0.00 | 3A4 |
| ATOM | 550 | CA | ILE | 119 | 31.251 | 6.157 | 27.657 | 1.00 | 0.00 | 3A4 |
| ATOM | 551 | CB | ILE | 119 | 31.480 | 4.637 | 27.705 | 1.00 | 0.00 | 3A4 |
| ATOM | 552 | CG2 | ILE | 119 | 32.895 | 4.296 | 28.225 | 1.00 | 0.00 | 3A4 |
| ATOM | 553 | CG1 | ILE | 119 | 30.373 | 3.794 | 28.400 | 1.00 | 0.00 | 3A4 |
| ATOM | 554 | CD | ILE | 119 | 29.071 | 3.681 | 27.605 | 1.00 | 0.00 | 3A4 |
| ATOM | 555 | C | ILE | 119 | 31.475 | 6.827 | 29.005 | 1.00 | 0.00 | 3A4 |
| ATOM | 556 | O | ILE | 119 | 30.894 | 6.426 | 30.013 | 1.00 | 0.00 | 3A4 |
| ATOM | 557 | N | ALA | 120 | 32.350 | 7.862 | 29.050 | 1.00 | 0.00 | 3A4 |
| ATOM | 558 | CA | ALA | 120 | 32.876 | 8.420 | 30.276 | 1.00 | 0.00 | 3A4 |
| ATOM | 559 | CB | ALA | 120 | 34.429 | 8.417 | 30.275 | 1.00 | 0.00 | 3A4 |
| ATOM | 560 | C | ALA | 120 | 32.403 | 9.821 | 30.537 | 1.00 | 0.00 | 3A4 |
| ATOM | 561 | O | ALA | 120 | 32.153 | 10.624 | 29.642 | 1.00 | 0.00 | 3A4 |
| ATOM | 562 | N | GLU | 121 | 32.338 | 10.132 | 31.847 | 1.00 | 0.00 | 3A4 |
| ATOM | 563 | CA | GLU | 121 | 31.987 | 11.402 | 32.421 | 1.00 | 0.00 | 3A4 |
| ATOM | 564 | CB | GLU | 121 | 31.189 | 11.213 | 33.742 | 1.00 | 0.00 | 3A4 |
| ATOM | 565 | CG | GLU | 121 | 29.884 | 10.420 | 33.613 | 1.00 | 0.00 | 3A4 |
| ATOM | 566 | CD | GLU | 121 | 30.124 | 8.902 | 33.618 | 1.00 | 0.00 | 3A4 |
| ATOM | 567 | OE1 | GLU | 121 | 29.800 | 8.241 | 32.594 | 1.00 | 0.00 | 3A4 |
| ATOM | 568 | OE2 | GLU | 121 | 30.631 | 8.385 | 34.649 | 1.00 | 0.00 | 3A4 |
| ATOM | 569 | C | GLU | 121 | 33.282 | 12.104 | 32.738 | 1.00 | 0.00 | 3A4 |
| ATOM | 570 | O | GLU | 121 | 34.307 | 11.460 | 32.947 | 1.00 | 0.00 | 3A4 |
| ATOM | 571 | N | ASP | 122 | 33.267 | 13.450 | 32.868 | 1.00 | 0.00 | 3A4 |
| ATOM | 572 | CA | ASP | 122 | 34.366 | 14.255 | 33.384 | 1.00 | 0.00 | 3A4 |
| ATOM | 573 | CB | ASP | 122 | 33.940 | 15.762 | 33.320 | 1.00 | 0.00 | 3A4 |
| ATOM | 574 | CG | ASP | 122 | 35.057 | 16.786 | 33.606 | 1.00 | 0.00 | 3A4 |
| ATOM | 575 | OD1 | ASP | 122 | 34.911 | 17.556 | 34.593 | 1.00 | 0.00 | 3A4 |
| ATOM | 576 | OD2 | ASP | 122 | 36.057 | 16.817 | 32.842 | 1.00 | 0.00 | 3A4 |
| ATOM | 577 | C | ASP | 122 | 34.615 | 13.879 | 34.870 | 1.00 | 0.00 | 3A4 |
| ATOM | 578 | O | ASP | 122 | 33.636 | 13.610 | 35.566 | 1.00 | 0.00 | 3A4 |
| ATOM | 579 | N | GLU | 123 | 35.863 | 13.792 | 35.421 | 1.00 | 0.00 | 3A4 |
| ATOM | 580 | CA | GLU | 123 | 37.162 | 14.248 | 34.972 | 1.00 | 0.00 | 3A4 |
| ATOM | 581 | CB | GLU | 123 | 37.993 | 14.687 | 36.197 | 1.00 | 0.00 | 3A4 |
| ATOM | 582 | CG | GLU | 123 | 37.348 | 15.854 | 36.968 | 1.00 | 0.00 | 3A4 |
| ATOM | 583 | CD | GLU | 123 | 38.253 | 16.265 | 38.134 | 1.00 | 0.00 | 3A4 |
| ATOM | 584 | OE1 | GLU | 123 | 38.764 | 17.417 | 38.116 | 1.00 | 0.00 | 3A4 |
| ATOM | 585 | OE2 | GLU | 123 | 38.443 | 15.431 | 39.060 | 1.00 | 0.00 | 3A4 |
| ATOM | 586 | C | GLU | 123 | 37.950 | 13.205 | 34.211 | 1.00 | 0.00 | 3A4 |
| ATOM | 587 | O | GLU | 123 | 39.000 | 13.494 | 33.638 | 1.00 | 0.00 | 3A4 |
| ATOM | 588 | N | GLU | 124 | 37.449 | 11.951 | 34.149 | 1.00 | 0.00 | 3A4 |
| ATOM | 589 | CA | GLU | 124 | 38.063 | 10.827 | 33.460 | 1.00 | 0.00 | 3A4 |
| ATOM | 590 | CB | GLU | 124 | 37.284 | 9.523 | 33.756 | 1.00 | 0.00 | 3A4 |
| ATOM | 591 | CG | GLU | 124 | 37.186 | 9.213 | 35.261 | 1.00 | 0.00 | 3A4 |
| ATOM | 592 | CD | GLU | 124 | 36.410 | 7.908 | 35.465 | 1.00 | 0.00 | 3A4 |
| ATOM | 593 | OE1 | GLU | 124 | 37.010 | 6.933 | 35.993 | 1.00 | 0.00 | 3A4 |
| ATOM | 594 | OE2 | GLU | 124 | 35.204 | 7.870 | 35.096 | 1.00 | 0.00 | 3A4 |
| ATOM | 595 | C | GLU | 124 | 38.109 | 11.000 | 31.965 | 1.00 | 0.00 | 3A4 |
| ATOM | 596 | O | GLU | 124 | 39.087 | 10.727 | 31.279 | 1.00 | 0.00 | 3A4 |
| ATOM | 597 | N | TRP | 125 | 37.027 | 11.579 | 31.425 | 1.00 | 0.00 | 3A4 |
| ATOM | 598 | CA | TRP | 125 | 36.896 | 11.901 | 30.029 | 1.00 | 0.00 | 3A4 |
| ATOM | 599 | CB | TRP | 125 | 35.456 | 12.317 | 29.760 | 1.00 | 0.00 | 3A4 |
| ATOM | 600 | CG | TRP | 125 | 35.096 | 13.136 | 28.553 | 1.00 | 0.00 | 3A4 |
| ATOM | 601 | CD2 | TRP | 125 | 34.736 | 14.501 | 28.811 | 1.00 | 0.00 | 3A4 |
| ATOM | 602 | CD1 | TRP | 125 | 34.423 | 12.765 | 27.428 | 1.00 | 0.00 | 3A4 |
| ATOM | 603 | NE1 | TRP | 125 | 33.701 | 13.815 | 26.942 | 1.00 | 0.00 | 3A4 |
| ATOM | 604 | CE2 | TRP | 125 | 33.805 | 14.860 | 27.829 | 1.00 | 0.00 | 3A4 |
| ATOM | 605 | CE3 | TRP | 125 | 35.040 | 15.361 | 29.870 | 1.00 | 0.00 | 3A4 |
| ATOM | 606 | CZ2 | TRP | 125 | 33.124 | 16.059 | 27.921 | 1.00 | 0.00 | 3A4 |
| ATOM | 607 | CZ3 | TRP | 125 | 34.396 | 16.599 | 29.921 | 1.00 | 0.00 | 3A4 |
| ATOM | 608 | CH2 | TRP | 125 | 33.421 | 16.924 | 28.984 | 1.00 | 0.00 | 3A4 |
| ATOM | 609 | C | TRP | 125 | 37.893 | 12.940 | 29.585 | 1.00 | 0.00 | 3A4 |
| ATOM | 610 | O | TRP | 125 | 38.564 | 12.734 | 28.590 | 1.00 | 0.00 | 3A4 |
| ATOM | 611 | N | LYS | 126 | 38.066 | 14.068 | 30.315 | 1.00 | 0.00 | 3A4 |
| ATOM | 612 | CA | LYS | 126 | 38.948 | 15.161 | 29.915 | 1.00 | 0.00 | 3A4 |
| ATOM | 613 | CB | LYS | 126 | 38.776 | 16.362 | 30.855 | 1.00 | 0.00 | 3A4 |
| ATOM | 614 | CG | LYS | 126 | 39.424 | 17.682 | 30.406 | 1.00 | 0.00 | 3A4 |
| ATOM | 615 | CD | LYS | 126 | 39.026 | 18.858 | 31.300 | 1.00 | 0.00 | 3A4 |
| ATOM | 616 | CE | LYS | 126 | 39.634 | 20.191 | 30.845 | 1.00 | 0.00 | 3A4 |
| ATOM | 617 | NZ | LYS | 126 | 39.206 | 21.296 | 31.735 | 1.00 | 0.00 | 3A4 |
| ATOM | 618 | C | LYS | 126 | 40.413 | 14.775 | 29.867 | 1.00 | 0.00 | 3A4 |
| ATOM | 619 | O | LYS | 126 | 41.152 | 15.173 | 28.977 | 1.00 | 0.00 | 3A4 |
| ATOM | 620 | N | ARG | 127 | 40.830 | 13.884 | 30.785 | 1.00 | 0.00 | 3A4 |
| ATOM | 621 | CA | ARG | 127 | 42.152 | 13.315 | 30.821 | 1.00 | 0.00 | 3A4 |
| ATOM | 622 | CB | ARG | 127 | 42.369 | 12.556 | 32.148 | 1.00 | 0.00 | 3A4 |
| ATOM | 623 | CG | ARG | 127 | 42.319 | 13.460 | 33.385 | 1.00 | 0.00 | 3A4 |
| ATOM | 624 | CD | ARG | 127 | 42.230 | 12.652 | 34.686 | 1.00 | 0.00 | 3A4 |
| ATOM | 625 | NE | ARG | 127 | 42.079 | 13.613 | 35.835 | 1.00 | 0.00 | 3A4 |
| ATOM | 626 | CZ | ARG | 127 | 41.521 | 13.267 | 37.040 | 1.00 | 0.00 | 3A4 |
| ATOM | 627 | NH1 | ARG | 127 | 41.438 | 14.206 | 38.026 | 1.00 | 0.00 | 3A4 |
| ATOM | 628 | NH2 | ARG | 127 | 41.040 | 12.009 | 37.276 | 1.00 | 0.00 | 3A4 |
| ATOM | 629 | C | ARG | 127 | 42.448 | 12.378 | 29.678 | 1.00 | 0.00 | 3A4 |
| ATOM | 630 | O | ARG | 127 | 43.503 | 12.464 | 29.060 | 1.00 | 0.00 | 3A4 |
| ATOM | 631 | N | LEU | 128 | 41.487 | 11.489 | 29.329 | 1.00 | 0.00 | 3A4 |
| ATOM | 632 | CA | LEU | 128 | 41.614 | 10.553 | 28.227 | 1.00 | 0.00 | 3A4 |
| ATOM | 633 | CB | LEU | 128 | 40.552 | 9.436 | 28.305 | 1.00 | 0.00 | 3A4 |
| ATOM | 634 | CG | LEU | 128 | 40.710 | 8.475 | 29.510 | 1.00 | 0.00 | 3A4 |
| ATOM | 635 | CD1 | LEU | 128 | 39.492 | 7.536 | 29.620 | 1.00 | 0.00 | 3A4 |
| ATOM | 636 | CD2 | LEU | 128 | 42.026 | 7.670 | 29.503 | 1.00 | 0.00 | 3A4 |
| ATOM | 637 | C | LEU | 128 | 41.513 | 11.260 | 26.900 | 1.00 | 0.00 | 3A4 |
| ATOM | 638 | O | LEU | 128 | 42.307 | 10.998 | 26.016 | 1.00 | 0.00 | 3A4 |
| ATOM | 639 | N | ARG | 129 | 40.613 | 12.256 | 26.764 | 1.00 | 0.00 | 3A4 |
| ATOM | 640 | CA | ARG | 129 | 40.455 | 13.056 | 25.570 | 1.00 | 0.00 | 3A4 |
| ATOM | 641 | CB | ARG | 129 | 39.248 | 13.995 | 25.706 | 1.00 | 0.00 | 3A4 |
| ATOM | 642 | CG | ARG | 129 | 38.865 | 14.698 | 24.411 | 1.00 | 0.00 | 3A4 |
| ATOM | 643 | CD | ARG | 129 | 37.443 | 15.285 | 24.477 | 1.00 | 0.00 | 3A4 |
| ATOM | 644 | NE | ARG | 129 | 36.965 | 15.632 | 23.089 | 1.00 | 0.00 | 3A4 |
| ATOM | 645 | CZ | ARG | 129 | 37.094 | 16.870 | 22.512 | 1.00 | 0.00 | 3A4 |
| ATOM | 646 | NH1 | ARG | 129 | 36.589 | 17.069 | 21.260 | 1.00 | 0.00 | 3A4 |
| ATOM | 647 | NH2 | ARG | 129 | 37.709 | 17.907 | 23.155 | 1.00 | 0.00 | 3A4 |
| ATOM | 648 | C | ARG | 129 | 41.673 | 13.896 | 25.247 | 1.00 | 0.00 | 3A4 |
| ATOM | 649 | O | ARG | 129 | 42.125 | 13.963 | 24.109 | 1.00 | 0.00 | 3A4 |
| ATOM | 650 | N | SER | 130 | 42.292 | 14.491 | 26.294 | 1.00 | 0.00 | 3A4 |
| ATOM | 651 | CA | SER | 130 | 43.486 | 15.299 | 26.191 | 1.00 | 0.00 | 3A4 |
| ATOM | 652 | CB | SER | 130 | 43.870 | 16.030 | 27.515 | 1.00 | 0.00 | 3A4 |
| ATOM | 653 | OG | SER | 130 | 42.925 | 17.049 | 27.812 | 1.00 | 0.00 | 3A4 |
| ATOM | 654 | C | SER | 130 | 44.691 | 14.547 | 25.703 | 1.00 | 0.00 | 3A4 |
| ATOM | 655 | O | SER | 130 | 45.506 | 15.121 | 25.002 | 1.00 | 0.00 | 3A4 |
| ATOM | 656 | N | LEU | 131 | 44.822 | 13.241 | 26.004 | 1.00 | 0.00 | 3A4 |
| ATOM | 657 | CA | LEU | 131 | 45.894 | 12.396 | 25.508 | 1.00 | 0.00 | 3A4 |
| ATOM | 658 | CB | LEU | 131 | 45.835 | 11.013 | 26.195 | 1.00 | 0.00 | 3A4 |
| ATOM | 659 | CG | LEU | 131 | 46.473 | 11.008 | 27.591 | 1.00 | 0.00 | 3A4 |
| ATOM | 660 | CD1 | LEU | 131 | 45.836 | 9.924 | 28.474 | 1.00 | 0.00 | 3A4 |
| ATOM | 661 | CD2 | LEU | 131 | 48.005 | 10.845 | 27.482 | 1.00 | 0.00 | 3A4 |
| ATOM | 662 | C | LEU | 131 | 45.875 | 12.189 | 24.006 | 1.00 | 0.00 | 3A4 |
| ATOM | 663 | O | LEU | 131 | 46.913 | 12.177 | 23.361 | 1.00 | 0.00 | 3A4 |
| ATOM | 664 | N | LEU | 132 | 44.675 | 12.059 | 23.421 | 1.00 | 0.00 | 3A4 |
| ATOM | 665 | CA | LEU | 132 | 44.436 | 11.692 | 22.040 | 1.00 | 0.00 | 3A4 |
| ATOM | 666 | CB | LEU | 132 | 43.113 | 10.883 | 21.884 | 1.00 | 0.00 | 3A4 |
| ATOM | 667 | CG | LEU | 132 | 42.628 | 10.220 | 23.187 | 1.00 | 0.00 | 3A4 |
| ATOM | 668 | CD1 | LEU | 132 | 41.170 | 9.739 | 23.225 | 1.00 | 0.00 | 3A4 |
| ATOM | 669 | CD2 | LEU | 132 | 43.579 | 9.147 | 23.759 | 1.00 | 0.00 | 3A4 |
| ATOM | 670 | C | LEU | 132 | 44.339 | 12.896 | 21.142 | 1.00 | 0.00 | 3A4 |
| ATOM | 671 | O | LEU | 132 | 44.709 | 12.862 | 19.975 | 1.00 | 0.00 | 3A4 |
| ATOM | 672 | N | SER | 133 | 43.808 | 14.009 | 21.685 | 1.00 | 0.00 | 3A4 |
| ATOM | 673 | CA | SER | 133 | 43.570 | 15.239 | 20.967 | 1.00 | 0.00 | 3A4 |
| ATOM | 674 | CB | SER | 133 | 42.882 | 16.277 | 21.854 | 1.00 | 0.00 | 3A4 |
| ATOM | 675 | OG | SER | 133 | 43.525 | 16.664 | 23.058 | 1.00 | 0.00 | 3A4 |
| ATOM | 676 | C | SER | 133 | 44.766 | 15.878 | 20.280 | 1.00 | 0.00 | 3A4 |
| ATOM | 677 | O | SER | 133 | 44.586 | 16.429 | 19.192 | 1.00 | 0.00 | 3A4 |
| ATOM | 678 | N | PRO | 134 | 46.017 | 15.798 | 20.777 | 1.00 | 0.00 | 3A4 |
| ATOM | 679 | CA | PRO | 134 | 47.159 | 16.327 | 20.076 | 1.00 | 0.00 | 3A4 |
| ATOM | 680 | CD | PRO | 134 | 46.405 | 15.537 | 22.155 | 1.00 | 0.00 | 3A4 |
| ATOM | 681 | CB | PRO | 134 | 48.327 | 16.318 | 21.087 | 1.00 | 0.00 | 3A4 |
| ATOM | 682 | CG | PRO | 134 | 47.635 | 16.387 | 22.434 | 1.00 | 0.00 | 3A4 |
| ATOM | 683 | C | PRO | 134 | 47.567 | 15.563 | 18.844 | 1.00 | 0.00 | 3A4 |
| ATOM | 684 | O | PRO | 134 | 48.106 | 16.175 | 17.927 | 1.00 | 0.00 | 3A4 |
| ATOM | 685 | N | THR | 135 | 47.305 | 14.235 | 18.803 | 1.00 | 0.00 | 3A4 |
| ATOM | 686 | CA | THR | 135 | 47.657 | 13.342 | 17.727 | 1.00 | 0.00 | 3A4 |
| ATOM | 687 | CB | THR | 135 | 47.864 | 11.912 | 18.232 | 1.00 | 0.00 | 3A4 |
| ATOM | 688 | OG1 | THR | 135 | 46.681 | 11.299 | 18.729 | 1.00 | 0.00 | 3A4 |
| ATOM | 689 | CG2 | THR | 135 | 48.889 | 11.953 | 19.387 | 1.00 | 0.00 | 3A4 |
| ATOM | 690 | C | THR | 135 | 46.639 | 13.419 | 16.594 | 1.00 | 0.00 | 3A4 |
| ATOM | 691 | O | THR | 135 | 46.921 | 13.057 | 15.460 | 1.00 | 0.00 | 3A4 |
| ATOM | 692 | N | PHE | 136 | 45.420 | 13.916 | 16.895 | 1.00 | 0.00 | 3A4 |
| ATOM | 693 | CA | PHE | 136 | 44.322 | 13.979 | 15.962 | 1.00 | 0.00 | 3A4 |
| ATOM | 694 | CB | PHE | 136 | 43.007 | 13.434 | 16.581 | 1.00 | 0.00 | 3A4 |
| ATOM | 695 | CG | PHE | 136 | 43.069 | 12.028 | 17.109 | 1.00 | 0.00 | 3A4 |
| ATOM | 696 | CD1 | PHE | 136 | 42.397 | 11.733 | 18.297 | 1.00 | 0.00 | 3A4 |
| ATOM | 697 | CD2 | PHE | 136 | 43.793 | 10.992 | 16.495 | 1.00 | 0.00 | 3A4 |
| ATOM | 698 | CE1 | PHE | 136 | 42.237 | 10.417 | 18.717 | 1.00 | 0.00 | 3A4 |
| ATOM | 699 | CE2 | PHE | 136 | 43.668 | 9.665 | 16.926 | 1.00 | 0.00 | 3A4 |
| ATOM | 700 | CZ | PHE | 136 | 42.847 | 9.372 | 18.017 | 1.00 | 0.00 | 3A4 |
| ATOM | 701 | C | PHE | 136 | 44.074 | 15.402 | 15.465 | 1.00 | 0.00 | 3A4 |
| ATOM | 702 | O | PHE | 136 | 42.971 | 15.703 | 15.025 | 1.00 | 0.00 | 3A4 |
| ATOM | 703 | N | THR | 137 | 45.088 | 16.323 | 15.493 | 1.00 | 0.00 | 3A4 |
| ATOM | 704 | CA | THR | 137 | 44.944 | 17.764 | 15.224 | 1.00 | 0.00 | 3A4 |
| ATOM | 705 | CB | THR | 137 | 46.098 | 18.572 | 15.830 | 1.00 | 0.00 | 3A4 |
| ATOM | 706 | OG1 | THR | 137 | 46.156 | 18.321 | 17.228 | 1.00 | 0.00 | 3A4 |
| ATOM | 707 | CG2 | THR | 137 | 45.936 | 20.108 | 15.640 | 1.00 | 0.00 | 3A4 |
| ATOM | 708 | C | THR | 137 | 44.796 | 18.094 | 13.739 | 1.00 | 0.00 | 3A4 |
| ATOM | 709 | O | THR | 137 | 43.885 | 18.830 | 13.359 | 1.00 | 0.00 | 3A4 |
| ATOM | 710 | N | SER | 138 | 45.685 | 17.534 | 12.870 | 1.00 | 0.00 | 3A4 |
| ATOM | 711 | CA | SER | 138 | 45.656 | 17.677 | 11.419 | 1.00 | 0.00 | 3A4 |
| ATOM | 712 | CB | SER | 138 | 47.076 | 17.874 | 10.806 | 1.00 | 0.00 | 3A4 |
| ATOM | 713 | OG | SER | 138 | 47.648 | 19.086 | 11.280 | 1.00 | 0.00 | 3A4 |
| ATOM | 714 | C | SER | 138 | 45.012 | 16.431 | 10.860 | 1.00 | 0.00 | 3A4 |
| ATOM | 715 | O | SER | 138 | 43.868 | 16.450 | 10.408 | 1.00 | 0.00 | 3A4 |
| ATOM | 716 | N | GLY | 139 | 45.756 | 15.304 | 10.953 | 1.00 | 0.00 | 3A4 |
| ATOM | 717 | CA | GLY | 139 | 45.288 | 13.957 | 10.734 | 1.00 | 0.00 | 3A4 |
| ATOM | 718 | C | GLY | 139 | 45.698 | 13.207 | 11.960 | 1.00 | 0.00 | 3A4 |
| ATOM | 719 | O | GLY | 139 | 46.061 | 13.818 | 12.961 | 1.00 | 0.00 | 3A4 |
| ATOM | 720 | N | LYS | 140 | 45.629 | 11.847 | 11.918 | 1.00 | 0.00 | 3A4 |
| ATOM | 721 | CA | LYS | 140 | 45.777 | 10.945 | 13.054 | 1.00 | 0.00 | 3A4 |
| ATOM | 722 | CB | LYS | 140 | 44.729 | 9.786 | 13.048 | 1.00 | 0.00 | 3A4 |
| ATOM | 723 | CG | LYS | 140 | 43.246 | 10.097 | 13.336 | 1.00 | 0.00 | 3A4 |
| ATOM | 724 | CD | LYS | 140 | 42.846 | 11.560 | 13.252 | 1.00 | 0.00 | 3A4 |
| ATOM | 725 | CE | LYS | 140 | 41.456 | 11.872 | 13.770 | 1.00 | 0.00 | 3A4 |
| ATOM | 726 | NZ | LYS | 140 | 41.193 | 13.331 | 13.606 | 1.00 | 0.00 | 3A4 |
| ATOM | 727 | C | LYS | 140 | 47.165 | 10.340 | 13.065 | 1.00 | 0.00 | 3A4 |
| ATOM | 728 | O | LYS | 140 | 47.330 | 9.123 | 12.976 | 1.00 | 0.00 | 3A4 |
| ATOM | 729 | N | LEU | 141 | 48.195 | 11.212 | 13.185 | 1.00 | 0.00 | 3A4 |
| ATOM | 730 | CA | LEU | 141 | 49.585 | 10.835 | 13.207 | 1.00 | 0.00 | 3A4 |
| ATOM | 731 | CB | LEU | 141 | 50.096 | 10.321 | 11.825 | 1.00 | 0.00 | 3A4 |
| ATOM | 732 | CG | LEU | 141 | 51.411 | 9.530 | 11.812 | 1.00 | 0.00 | 3A4 |
| ATOM | 733 | CD1 | LEU | 141 | 52.059 | 9.603 | 10.425 | 1.00 | 0.00 | 3A4 |
| ATOM | 734 | CD2 | LEU | 141 | 51.161 | 8.066 | 12.211 | 1.00 | 0.00 | 3A4 |
| ATOM | 735 | C | LEU | 141 | 50.286 | 12.100 | 13.623 | 1.00 | 0.00 | 3A4 |
| ATOM | 736 | O | LEU | 141 | 50.455 | 12.362 | 14.813 | 1.00 | 0.00 | 3A4 |
| ATOM | 737 | N | LYS | 142 | 50.687 | 12.924 | 12.618 | 1.00 | 0.00 | 3A4 |
| ATOM | 738 | CA | LYS | 142 | 51.307 | 14.222 | 12.760 | 1.00 | 0.00 | 3A4 |
| ATOM | 739 | CB | LYS | 142 | 52.868 | 14.192 | 12.729 | 1.00 | 0.00 | 3A4 |
| ATOM | 740 | CG | LYS | 142 | 53.516 | 13.465 | 13.915 | 1.00 | 0.00 | 3A4 |
| ATOM | 741 | CD | LYS | 142 | 55.045 | 13.555 | 13.916 | 1.00 | 0.00 | 3A4 |
| ATOM | 742 | CE | LYS | 142 | 55.714 | 12.858 | 15.108 | 1.00 | 0.00 | 3A4 |
| ATOM | 743 | NZ | LYS | 142 | 55.454 | 11.401 | 15.086 | 1.00 | 0.00 | 3A4 |
| ATOM | 744 | C | LYS | 142 | 50.813 | 15.068 | 11.606 | 1.00 | 0.00 | 3A4 |
| ATOM | 745 | O | LYS | 142 | 50.704 | 16.287 | 11.736 | 1.00 | 0.00 | 3A4 |
| ATOM | 746 | N | GLU | 143 | 50.529 | 14.432 | 10.433 | 1.00 | 0.00 | 3A4 |
| ATOM | 747 | CA | GLU | 143 | 50.169 | 15.105 | 9.198 | 1.00 | 0.00 | 3A4 |
| ATOM | 748 | CB | GLU | 143 | 51.389 | 15.242 | 8.234 | 1.00 | 0.00 | 3A4 |
| ATOM | 749 | CG | GLU | 143 | 51.158 | 16.046 | 6.935 | 1.00 | 0.00 | 3A4 |
| ATOM | 750 | CD | GLU | 143 | 50.774 | 17.493 | 7.267 | 1.00 | 0.00 | 3A4 |
| ATOM | 751 | OE1 | GLU | 143 | 51.602 | 18.194 | 7.909 | 1.00 | 0.00 | 3A4 |
| ATOM | 752 | OE2 | GLU | 143 | 49.651 | 17.917 | 6.882 | 1.00 | 0.00 | 3A4 |
| ATOM | 753 | C | GLU | 143 | 49.047 | 14.324 | 8.559 | 1.00 | 0.00 | 3A4 |
| ATOM | 754 | O | GLU | 143 | 47.898 | 14.760 | 8.605 | 1.00 | 0.00 | 3A4 |
| ATOM | 755 | N | MET | 144 | 49.393 | 13.160 | 7.926 | 1.00 | 0.00 | 3A4 |
| ATOM | 756 | CA | MET | 144 | 48.584 | 12.130 | 7.300 | 1.00 | 0.00 | 3A4 |
| ATOM | 757 | CB | MET | 144 | 47.303 | 11.759 | 8.070 | 1.00 | 0.00 | 3A4 |
| ATOM | 758 | CG | MET | 144 | 47.567 | 10.932 | 9.334 | 1.00 | 0.00 | 3A4 |
| ATOM | 759 | SD | MET | 144 | 48.269 | 9.267 | 9.019 | 1.00 | 0.00 | 3A4 |
| ATOM | 760 | CE | MET | 144 | 47.029 | 8.504 | 7.928 | 1.00 | 0.00 | 3A4 |
| ATOM | 761 | C | MET | 144 | 48.192 | 12.409 | 5.879 | 1.00 | 0.00 | 3A4 |
| ATOM | 762 | O | MET | 144 | 47.891 | 11.472 | 5.150 | 1.00 | 0.00 | 3A4 |
| ATOM | 763 | N | VAL | 145 | 48.169 | 13.678 | 5.428 | 1.00 | 0.00 | 3A4 |
| ATOM | 764 | CA | VAL | 145 | 47.650 | 14.088 | 4.130 | 1.00 | 0.00 | 3A4 |
| ATOM | 765 | CB | VAL | 145 | 47.693 | 15.597 | 3.949 | 1.00 | 0.00 | 3A4 |
| ATOM | 766 | CG1 | VAL | 145 | 46.839 | 16.061 | 2.734 | 1.00 | 0.00 | 3A4 |
| ATOM | 767 | CG2 | VAL | 145 | 47.128 | 16.245 | 5.237 | 1.00 | 0.00 | 3A4 |
| ATOM | 768 | C | VAL | 145 | 48.261 | 13.425 | 2.906 | 1.00 | 0.00 | 3A4 |
| ATOM | 769 | O | VAL | 145 | 47.501 | 13.014 | 2.026 | 1.00 | 0.00 | 3A4 |
| ATOM | 770 | N | PRO | 146 | 49.595 | 13.235 | 2.821 | 1.00 | 0.00 | 3A4 |
| ATOM | 771 | CA | PRO | 146 | 50.226 | 12.490 | 1.741 | 1.00 | 0.00 | 3A4 |
| ATOM | 772 | CD | PRO | 146 | 50.623 | 13.942 | 3.599 | 1.00 | 0.00 | 3A4 |
| ATOM | 773 | CB | PRO | 146 | 51.743 | 12.625 | 1.988 | 1.00 | 0.00 | 3A4 |
| ATOM | 774 | CG | PRO | 146 | 51.872 | 13.954 | 2.721 | 1.00 | 0.00 | 3A4 |
| ATOM | 775 | C | PRO | 146 | 49.826 | 11.029 | 1.698 | 1.00 | 0.00 | 3A4 |
| ATOM | 776 | O | PRO | 146 | 49.678 | 10.483 | 0.613 | 1.00 | 0.00 | 3A4 |
| ATOM | 777 | N | ILE | 147 | 49.572 | 10.388 | 2.863 | 1.00 | 0.00 | 3A4 |
| ATOM | 778 | CA | ILE | 147 | 49.098 | 9.027 | 2.976 | 1.00 | 0.00 | 3A4 |
| ATOM | 779 | CB | ILE | 147 | 49.254 | 8.469 | 4.382 | 1.00 | 0.00 | 3A4 |
| ATOM | 780 | CG2 | ILE | 147 | 48.993 | 6.930 | 4.422 | 1.00 | 0.00 | 3A4 |
| ATOM | 781 | CG1 | ILE | 147 | 50.685 | 8.809 | 4.901 | 1.00 | 0.00 | 3A4 |
| ATOM | 782 | CD | ILE | 147 | 51.018 | 8.281 | 6.300 | 1.00 | 0.00 | 3A4 |
| ATOM | 783 | C | ILE | 147 | 47.660 | 8.881 | 2.564 | 1.00 | 0.00 | 3A4 |
| ATOM | 784 | O | ILE | 147 | 47.306 | 7.899 | 1.922 | 1.00 | 0.00 | 3A4 |
| ATOM | 785 | N | ILE | 148 | 46.789 | 9.866 | 2.875 | 1.00 | 0.00 | 3A4 |
| ATOM | 786 | CA | ILE | 148 | 45.386 | 9.833 | 2.503 | 1.00 | 0.00 | 3A4 |
| ATOM | 787 | CB | ILE | 148 | 44.570 | 10.932 | 3.172 | 1.00 | 0.00 | 3A4 |
| ATOM | 788 | CG2 | ILE | 148 | 43.057 | 10.768 | 2.811 | 1.00 | 0.00 | 3A4 |
| ATOM | 789 | CG1 | ILE | 148 | 44.675 | 10.809 | 4.718 | 1.00 | 0.00 | 3A4 |
| ATOM | 790 | CD | ILE | 148 | 44.158 | 12.030 | 5.488 | 1.00 | 0.00 | 3A4 |
| ATOM | 791 | C | ILE | 148 | 45.218 | 9.888 | 1.005 | 1.00 | 0.00 | 3A4 |
| ATOM | 792 | O | ILE | 148 | 44.486 | 9.106 | 0.407 | 1.00 | 0.00 | 3A4 |
| ATOM | 793 | N | ALA | 149 | 46.000 | 10.765 | 0.350 | 1.00 | 0.00 | 3A4 |
| ATOM | 794 | CA | ALA | 149 | 46.058 | 10.923 | −1.086 | 1.00 | 0.00 | 3A4 |
| ATOM | 795 | CB | ALA | 149 | 46.983 | 12.097 | −1.414 | 1.00 | 0.00 | 3A4 |
| ATOM | 796 | C | ALA | 149 | 46.539 | 9.682 | −1.803 | 1.00 | 0.00 | 3A4 |
| ATOM | 797 | O | ALA | 149 | 45.965 | 9.246 | −2.797 | 1.00 | 0.00 | 3A4 |
| ATOM | 798 | N | GLN | 150 | 47.578 | 9.019 | −1.243 | 1.00 | 0.00 | 3A4 |
| ATOM | 799 | CA | GLN | 150 | 48.121 | 7.780 | −1.753 | 1.00 | 0.00 | 3A4 |
| ATOM | 800 | CB | GLN | 150 | 49.342 | 7.295 | −0.954 | 1.00 | 0.00 | 3A4 |
| ATOM | 801 | CG | GLN | 150 | 50.637 | 8.037 | −1.297 | 1.00 | 0.00 | 3A4 |
| ATOM | 802 | CD | GLN | 150 | 51.737 | 7.586 | −0.326 | 1.00 | 0.00 | 3A4 |
| ATOM | 803 | OE1 | GLN | 150 | 52.080 | 6.406 | −0.292 | 1.00 | 0.00 | 3A4 |
| ATOM | 804 | NE2 | GLN | 150 | 52.300 | 8.525 | 0.483 | 1.00 | 0.00 | 3A4 |
| ATOM | 805 | C | GLN | 150 | 47.129 | 6.663 | −1.774 | 1.00 | 0.00 | 3A4 |
| ATOM | 806 | O | GLN | 150 | 46.962 | 5.992 | −2.789 | 1.00 | 0.00 | 3A4 |
| ATOM | 807 | N | TYR | 151 | 46.371 | 6.500 | −0.658 | 1.00 | 0.00 | 3A4 |
| ATOM | 808 | CA | TYR | 151 | 45.418 | 5.431 | −0.538 | 1.00 | 0.00 | 3A4 |
| ATOM | 809 | CB | TYR | 151 | 44.853 | 5.227 | 0.859 | 1.00 | 0.00 | 3A4 |
| ATOM | 810 | CG | TYR | 151 | 44.181 | 3.823 | 0.880 | 1.00 | 0.00 | 3A4 |
| ATOM | 811 | CD1 | TYR | 151 | 43.188 | 3.417 | 1.745 | 1.00 | 0.00 | 3A4 |
| ATOM | 812 | CD2 | TYR | 151 | 44.795 | 2.750 | 0.124 | 1.00 | 0.00 | 3A4 |
| ATOM | 813 | CE1 | TYR | 151 | 42.443 | 2.274 | 1.345 | 1.00 | 0.00 | 3A4 |
| ATOM | 814 | CE2 | TYR | 151 | 44.170 | 1.533 | −0.113 | 1.00 | 0.00 | 3A4 |
| ATOM | 815 | CZ | TYR | 151 | 42.935 | 1.315 | 0.454 | 1.00 | 0.00 | 3A4 |
| ATOM | 816 | OH | TYR | 151 | 42.254 | 0.104 | 0.201 | 1.00 | 0.00 | 3A4 |
| ATOM | 817 | C | TYR | 151 | 44.221 | 5.669 | −1.449 | 1.00 | 0.00 | 3A4 |
| ATOM | 818 | O | TYR | 151 | 43.738 | 4.750 | −2.103 | 1.00 | 0.00 | 3A4 |
| ATOM | 819 | N | GLY | 152 | 43.775 | 6.936 | −1.554 | 1.00 | 0.00 | 3A4 |
| ATOM | 820 | CA | GLY | 152 | 42.703 | 7.368 | −2.423 | 1.00 | 0.00 | 3A4 |
| ATOM | 821 | C | GLY | 152 | 42.962 | 7.025 | −3.877 | 1.00 | 0.00 | 3A4 |
| ATOM | 822 | O | GLY | 152 | 42.122 | 6.466 | −4.565 | 1.00 | 0.00 | 3A4 |
| ATOM | 823 | N | ASP | 153 | 44.194 | 7.284 | −4.339 | 1.00 | 0.00 | 3A4 |
| ATOM | 824 | CA | ASP | 153 | 44.647 | 6.996 | −5.674 | 1.00 | 0.00 | 3A4 |
| ATOM | 825 | CB | ASP | 153 | 46.061 | 7.586 | −5.844 | 1.00 | 0.00 | 3A4 |
| ATOM | 826 | CG | ASP | 153 | 45.993 | 9.119 | −5.945 | 1.00 | 0.00 | 3A4 |
| ATOM | 827 | OD1 | ASP | 153 | 45.014 | 9.650 | −6.534 | 1.00 | 0.00 | 3A4 |
| ATOM | 828 | OD2 | ASP | 153 | 46.922 | 9.784 | −5.416 | 1.00 | 0.00 | 3A4 |
| ATOM | 829 | C | ASP | 153 | 44.703 | 5.521 | −6.007 | 1.00 | 0.00 | 3A4 |
| ATOM | 830 | O | ASP | 153 | 44.288 | 5.096 | −7.076 | 1.00 | 0.00 | 3A4 |
| ATOM | 831 | N | VAL | 154 | 45.154 | 4.686 | −5.048 | 1.00 | 0.00 | 3A4 |
| ATOM | 832 | CA | VAL | 154 | 45.173 | 3.235 | −5.160 | 1.00 | 0.00 | 3A4 |
| ATOM | 833 | CB | VAL | 154 | 45.878 | 2.605 | −3.968 | 1.00 | 0.00 | 3A4 |
| ATOM | 834 | CG1 | VAL | 154 | 45.770 | 1.054 | −3.875 | 1.00 | 0.00 | 3A4 |
| ATOM | 835 | CG2 | VAL | 154 | 47.373 | 2.989 | −4.067 | 1.00 | 0.00 | 3A4 |
| ATOM | 836 | C | VAL | 154 | 43.793 | 2.651 | −5.283 | 1.00 | 0.00 | 3A4 |
| ATOM | 837 | O | VAL | 154 | 43.531 | 1.783 | −6.112 | 1.00 | 0.00 | 3A4 |
| ATOM | 838 | N | LEU | 155 | 42.842 | 3.184 | −4.494 | 1.00 | 0.00 | 3A4 |
| ATOM | 839 | CA | LEU | 155 | 41.460 | 2.758 | −4.505 | 1.00 | 0.00 | 3A4 |
| ATOM | 840 | CB | LEU | 155 | 40.697 | 3.480 | −3.395 | 1.00 | 0.00 | 3A4 |
| ATOM | 841 | CG | LEU | 155 | 41.005 | 3.018 | −1.989 | 1.00 | 0.00 | 3A4 |
| ATOM | 842 | CD1 | LEU | 155 | 40.785 | 4.226 | −1.096 | 1.00 | 0.00 | 3A4 |
| ATOM | 843 | CD2 | LEU | 155 | 40.117 | 1.825 | −1.591 | 1.00 | 0.00 | 3A4 |
| ATOM | 844 | C | LEU | 155 | 40.732 | 3.082 | −5.782 | 1.00 | 0.00 | 3A4 |
| ATOM | 845 | O | LEU | 155 | 39.955 | 2.295 | −6.313 | 1.00 | 0.00 | 3A4 |
| ATOM | 846 | N | VAL | 156 | 41.020 | 4.276 | −6.333 | 1.00 | 0.00 | 3A4 |
| ATOM | 847 | CA | VAL | 156 | 40.441 | 4.744 | −7.564 | 1.00 | 0.00 | 3A4 |
| ATOM | 848 | CB | VAL | 156 | 40.709 | 6.216 | −7.769 | 1.00 | 0.00 | 3A4 |
| ATOM | 849 | CG1 | VAL | 156 | 40.208 | 6.709 | −9.147 | 1.00 | 0.00 | 3A4 |
| ATOM | 850 | CG2 | VAL | 156 | 39.887 | 6.989 | −6.709 | 1.00 | 0.00 | 3A4 |
| ATOM | 851 | C | VAL | 156 | 40.956 | 3.916 | −8.726 | 1.00 | 0.00 | 3A4 |
| ATOM | 852 | O | VAL | 156 | 40.186 | 3.474 | −9.566 | 1.00 | 0.00 | 3A4 |
| ATOM | 853 | N | ARG | 157 | 42.263 | 3.571 | −8.714 | 1.00 | 0.00 | 3A4 |
| ATOM | 854 | CA | ARG | 157 | 42.881 | 2.692 | −9.687 | 1.00 | 0.00 | 3A4 |
| ATOM | 855 | CB | ARG | 157 | 44.400 | 2.603 | −9.480 | 1.00 | 0.00 | 3A4 |
| ATOM | 856 | CG | ARG | 157 | 45.201 | 1.988 | −10.646 | 1.00 | 0.00 | 3A4 |
| ATOM | 857 | CD | ARG | 157 | 46.718 | 1.949 | −10.406 | 1.00 | 0.00 | 3A4 |
| ATOM | 858 | NE | ARG | 157 | 47.008 | 0.982 | −9.282 | 1.00 | 0.00 | 3A4 |
| ATOM | 859 | CZ | ARG | 157 | 47.582 | 1.328 | −8.084 | 1.00 | 0.00 | 3A4 |
| ATOM | 860 | NH1 | ARG | 157 | 47.753 | 0.362 | −7.136 | 1.00 | 0.00 | 3A4 |
| ATOM | 861 | NH2 | ARG | 157 | 47.975 | 2.607 | −7.807 | 1.00 | 0.00 | 3A4 |
| ATOM | 862 | C | ARG | 157 | 42.316 | 1.305 | −9.694 | 1.00 | 0.00 | 3A4 |
| ATOM | 863 | O | ARG | 157 | 42.062 | 0.746 | −10.749 | 1.00 | 0.00 | 3A4 |
| ATOM | 864 | N | ASN | 158 | 42.004 | 0.746 | −8.509 | 1.00 | 0.00 | 3A4 |
| ATOM | 865 | CA | ASN | 158 | 41.363 | −0.540 | −8.359 | 1.00 | 0.00 | 3A4 |
| ATOM | 866 | CB | ASN | 158 | 41.312 | −0.962 | −6.875 | 1.00 | 0.00 | 3A4 |
| ATOM | 867 | CG | ASN | 158 | 42.725 | −1.184 | −6.308 | 1.00 | 0.00 | 3A4 |
| ATOM | 868 | OD1 | ASN | 158 | 43.710 | −1.336 | −7.029 | 1.00 | 0.00 | 3A4 |
| ATOM | 869 | ND2 | ASN | 158 | 42.827 | −1.211 | −4.951 | 1.00 | 0.00 | 3A4 |
| ATOM | 870 | C | ASN | 158 | 39.959 | −0.569 | −8.928 | 1.00 | 0.00 | 3A4 |
| ATOM | 871 | O | ASN | 158 | 39.541 | −1.568 | −9.503 | 1.00 | 0.00 | 3A4 |
| ATOM | 872 | N | LEU | 159 | 39.205 | 0.547 | −8.840 | 1.00 | 0.00 | 3A4 |
| ATOM | 873 | CA | LEU | 159 | 37.900 | 0.698 | −9.460 | 1.00 | 0.00 | 3A4 |
| ATOM | 874 | CB | LEU | 159 | 37.181 | 1.944 | −8.901 | 1.00 | 0.00 | 3A4 |
| ATOM | 875 | CG | LEU | 159 | 36.392 | 1.845 | −7.590 | 1.00 | 0.00 | 3A4 |
| ATOM | 876 | CD1 | LEU | 159 | 35.846 | 3.252 | −7.275 | 1.00 | 0.00 | 3A4 |
| ATOM | 877 | CD2 | LEU | 159 | 35.226 | 0.844 | −7.672 | 1.00 | 0.00 | 3A4 |
| ATOM | 878 | C | LEU | 159 | 37.986 | 0.850 | −10.979 | 1.00 | 0.00 | 3A4 |
| ATOM | 879 | O | LEU | 159 | 37.180 | 0.311 | −11.731 | 1.00 | 0.00 | 3A4 |
| ATOM | 880 | N | ARG | 160 | 39.032 | 1.542 | −11.475 | 1.00 | 0.00 | 3A4 |
| ATOM | 881 | CA | ARG | 160 | 39.316 | 1.707 | −12.887 | 1.00 | 0.00 | 3A4 |
| ATOM | 882 | CB | ARG | 160 | 40.462 | 2.697 | −13.097 | 1.00 | 0.00 | 3A4 |
| ATOM | 883 | CG | ARG | 160 | 40.032 | 4.151 | −12.829 | 1.00 | 0.00 | 3A4 |
| ATOM | 884 | CD | ARG | 160 | 41.097 | 5.224 | −13.106 | 1.00 | 0.00 | 3A4 |
| ATOM | 885 | NE | ARG | 160 | 42.243 | 5.069 | −12.147 | 1.00 | 0.00 | 3A4 |
| ATOM | 886 | CZ | ARG | 160 | 43.077 | 6.096 | −11.785 | 1.00 | 0.00 | 3A4 |
| ATOM | 887 | NH1 | ARG | 160 | 44.114 | 5.859 | −10.932 | 1.00 | 0.00 | 3A4 |
| ATOM | 888 | NH2 | ARG | 160 | 42.889 | 7.363 | −12.251 | 1.00 | 0.00 | 3A4 |
| ATOM | 889 | C | ARG | 160 | 39.687 | 0.420 | −13.585 | 1.00 | 0.00 | 3A4 |
| ATOM | 890 | O | ARG | 160 | 39.428 | 0.250 | −14.767 | 1.00 | 0.00 | 3A4 |
| ATOM | 891 | N | ARG | 161 | 40.250 | −0.548 | −12.834 | 1.00 | 0.00 | 3A4 |
| ATOM | 892 | CA | ARG | 161 | 40.597 | −1.870 | −13.293 | 1.00 | 0.00 | 3A4 |
| ATOM | 893 | CB | ARG | 161 | 41.743 | −2.432 | −12.420 | 1.00 | 0.00 | 3A4 |
| ATOM | 894 | CG | ARG | 161 | 43.082 | −1.717 | −12.670 | 1.00 | 0.00 | 3A4 |
| ATOM | 895 | CD | ARG | 161 | 44.093 | −1.838 | −11.519 | 1.00 | 0.00 | 3A4 |
| ATOM | 896 | NE | ARG | 161 | 44.400 | −3.285 | −11.257 | 1.00 | 0.00 | 3A4 |
| ATOM | 897 | CZ | ARG | 161 | 45.172 | −3.692 | −10.198 | 1.00 | 0.00 | 3A4 |
| ATOM | 898 | NH1 | ARG | 161 | 45.396 | −5.024 | −10.010 | 1.00 | 0.00 | 3A4 |
| ATOM | 899 | NH2 | ARG | 161 | 45.720 | −2.792 | −9.328 | 1.00 | 0.00 | 3A4 |
| ATOM | 900 | C | ARG | 161 | 39.432 | −2.837 | −13.270 | 1.00 | 0.00 | 3A4 |
| ATOM | 901 | O | ARG | 161 | 39.618 | −4.026 | −13.474 | 1.00 | 0.00 | 3A4 |
| ATOM | 902 | N | GLU | 162 | 38.181 | −2.341 | −13.060 | 1.00 | 0.00 | 3A4 |
| ATOM | 903 | CA | GLU | 162 | 36.943 | −3.106 | −13.121 | 1.00 | 0.00 | 3A4 |
| ATOM | 904 | CB | GLU | 162 | 35.704 | −2.403 | −12.447 | 1.00 | 0.00 | 3A4 |
| ATOM | 905 | CG | GLU | 162 | 35.006 | −1.245 | −13.229 | 1.00 | 0.00 | 3A4 |
| ATOM | 906 | CD | GLU | 162 | 33.961 | −0.524 | −12.375 | 1.00 | 0.00 | 3A4 |
| ATOM | 907 | OE1 | GLU | 162 | 34.331 | −0.002 | −11.289 | 1.00 | 0.00 | 3A4 |
| ATOM | 908 | OE2 | GLU | 162 | 32.779 | −0.469 | −12.809 | 1.00 | 0.00 | 3A4 |
| ATOM | 909 | C | GLU | 162 | 36.578 | −3.419 | −14.538 | 1.00 | 0.00 | 3A4 |
| ATOM | 910 | O | GLU | 162 | 36.521 | −2.577 | −15.435 | 1.00 | 0.00 | 3A4 |
| ATOM | 911 | N | ALA | 163 | 36.386 | −4.746 | −14.788 | 1.00 | 0.00 | 3A4 |
| ATOM | 912 | CA | ALA | 163 | 36.408 | −5.391 | −16.091 | 1.00 | 0.00 | 3A4 |
| ATOM | 913 | CB | ALA | 163 | 37.029 | −6.809 | −15.993 | 1.00 | 0.00 | 3A4 |
| ATOM | 914 | C | ALA | 163 | 35.037 | −5.494 | −16.717 | 1.00 | 0.00 | 3A4 |
| ATOM | 915 | O | ALA | 163 | 34.019 | −5.308 | −16.051 | 1.00 | 0.00 | 3A4 |
| ATOM | 916 | N | GLU | 164 | 35.042 | −5.771 | −18.059 | 1.00 | 0.00 | 3A4 |
| ATOM | 917 | CA | GLU | 164 | 33.935 | −5.860 | −19.009 | 1.00 | 0.00 | 3A4 |
| ATOM | 918 | CB | GLU | 164 | 32.661 | −6.587 | −18.459 | 1.00 | 0.00 | 3A4 |
| ATOM | 919 | CG | GLU | 164 | 31.701 | −7.151 | −19.531 | 1.00 | 0.00 | 3A4 |
| ATOM | 920 | CD | GLU | 164 | 30.505 | −7.813 | −18.839 | 1.00 | 0.00 | 3A4 |
| ATOM | 921 | OE1 | GLU | 164 | 29.737 | −7.082 | −18.156 | 1.00 | 0.00 | 3A4 |
| ATOM | 922 | OE2 | GLU | 164 | 30.342 | −9.055 | −18.984 | 1.00 | 0.00 | 3A4 |
| ATOM | 923 | C | GLU | 164 | 33.612 | −4.461 | −19.520 | 1.00 | 0.00 | 3A4 |
| ATOM | 924 | O | GLU | 164 | 32.462 | −4.138 | −19.797 | 1.00 | 0.00 | 3A4 |
| ATOM | 925 | N | THR | 165 | 34.671 | −3.592 | −19.572 | 1.00 | 0.00 | 3A4 |
| ATOM | 926 | CA | THR | 165 | 34.730 | −2.131 | −19.714 | 1.00 | 0.00 | 3A4 |
| ATOM | 927 | CB | THR | 165 | 34.039 | −1.480 | −20.938 | 1.00 | 0.00 | 3A4 |
| ATOM | 928 | OG1 | THR | 165 | 32.615 | −1.557 | −20.965 | 1.00 | 0.00 | 3A4 |
| ATOM | 929 | CG2 | THR | 165 | 34.604 | −2.115 | −22.228 | 1.00 | 0.00 | 3A4 |
| ATOM | 930 | C | THR | 165 | 34.372 | −1.470 | −18.379 | 1.00 | 0.00 | 3A4 |
| ATOM | 931 | O | THR | 165 | 35.235 | −0.931 | −17.690 | 1.00 | 0.00 | 3A4 |
| ATOM | 932 | N | GLY | 166 | 33.075 | −1.547 | −17.996 | 1.00 | 0.00 | 3A4 |
| ATOM | 933 | CA | GLY | 166 | 32.567 | −1.129 | −16.720 | 1.00 | 0.00 | 3A4 |
| ATOM | 934 | C | GLY | 166 | 31.084 | −1.094 | −16.877 | 1.00 | 0.00 | 3A4 |
| ATOM | 935 | O | GLY | 166 | 30.541 | −0.151 | −17.449 | 1.00 | 0.00 | 3A4 |
| ATOM | 936 | N | LYS | 167 | 30.386 | −2.143 | −16.363 | 1.00 | 0.00 | 3A4 |
| ATOM | 937 | CA | LYS | 167 | 28.935 | −2.211 | −16.262 | 1.00 | 0.00 | 3A4 |
| ATOM | 938 | CB | LYS | 167 | 28.217 | −3.202 | −17.239 | 1.00 | 0.00 | 3A4 |
| ATOM | 939 | CG | LYS | 167 | 28.165 | −2.770 | −18.713 | 1.00 | 0.00 | 3A4 |
| ATOM | 940 | CD | LYS | 167 | 29.336 | −3.252 | −19.574 | 1.00 | 0.00 | 3A4 |
| ATOM | 941 | CE | LYS | 167 | 29.134 | −2.949 | −21.066 | 1.00 | 0.00 | 3A4 |
| ATOM | 942 | NZ | LYS | 167 | 30.208 | −3.554 | −21.890 | 1.00 | 0.00 | 3A4 |
| ATOM | 943 | C | LYS | 167 | 28.582 | −2.486 | −14.802 | 1.00 | 0.00 | 3A4 |
| ATOM | 944 | O | LYS | 167 | 27.816 | −1.688 | −14.271 | 1.00 | 0.00 | 3A4 |
| ATOM | 945 | N | PRO | 168 | 29.041 | −3.521 | −14.057 | 1.00 | 0.00 | 3A4 |
| ATOM | 946 | CA | PRO | 168 | 28.796 | −3.641 | −12.622 | 1.00 | 0.00 | 3A4 |
| ATOM | 947 | CD | PRO | 168 | 29.518 | −4.787 | −14.626 | 1.00 | 0.00 | 3A4 |
| ATOM | 948 | CB | PRO | 168 | 28.695 | −5.165 | −12.400 | 1.00 | 0.00 | 3A4 |
| ATOM | 949 | CG | PRO | 168 | 29.625 | −5.777 | −13.459 | 1.00 | 0.00 | 3A4 |
| ATOM | 950 | C | PRO | 168 | 29.938 | −3.027 | −11.812 | 1.00 | 0.00 | 3A4 |
| ATOM | 951 | O | PRO | 168 | 31.100 | −3.303 | −12.110 | 1.00 | 0.00 | 3A4 |
| ATOM | 952 | N | VAL | 169 | 29.609 | −2.275 | −10.724 | 1.00 | 0.00 | 3A4 |
| ATOM | 953 | CA | VAL | 169 | 30.441 | −2.280 | −9.531 | 1.00 | 0.00 | 3A4 |
| ATOM | 954 | CB | VAL | 169 | 31.674 | −1.381 | −9.591 | 1.00 | 0.00 | 3A4 |
| ATOM | 955 | CG1 | VAL | 169 | 31.356 | 0.130 | −9.641 | 1.00 | 0.00 | 3A4 |
| ATOM | 956 | CG2 | VAL | 169 | 32.702 | −1.746 | −8.502 | 1.00 | 0.00 | 3A4 |
| ATOM | 957 | C | VAL | 169 | 29.535 | −1.926 | −8.382 | 1.00 | 0.00 | 3A4 |
| ATOM | 958 | O | VAL | 169 | 28.623 | −1.120 | −8.526 | 1.00 | 0.00 | 3A4 |
| ATOM | 959 | N | THR | 170 | 29.805 | −2.498 | −7.184 | 1.00 | 0.00 | 3A4 |
| ATOM | 960 | CA | THR | 170 | 29.193 | −2.121 | −5.931 | 1.00 | 0.00 | 3A4 |
| ATOM | 961 | CB | THR | 170 | 28.902 | −3.314 | −5.038 | 1.00 | 0.00 | 3A4 |
| ATOM | 962 | OG1 | THR | 170 | 28.066 | −4.231 | −5.734 | 1.00 | 0.00 | 3A4 |
| ATOM | 963 | CG2 | THR | 170 | 28.173 | −2.877 | −3.736 | 1.00 | 0.00 | 3A4 |
| ATOM | 964 | C | THR | 170 | 30.178 | −1.183 | −5.305 | 1.00 | 0.00 | 3A4 |
| ATOM | 965 | O | THR | 170 | 31.232 | −1.589 | −4.831 | 1.00 | 0.00 | 3A4 |
| ATOM | 966 | N | LEU | 171 | 29.854 | 0.126 | −5.296 | 1.00 | 0.00 | 3A4 |
| ATOM | 967 | CA | LEU | 171 | 30.705 | 1.177 | −4.769 | 1.00 | 0.00 | 3A4 |
| ATOM | 968 | CB | LEU | 171 | 30.055 | 2.555 | −5.044 | 1.00 | 0.00 | 3A4 |
| ATOM | 969 | CG | LEU | 171 | 30.547 | 3.319 | −6.282 | 1.00 | 0.00 | 3A4 |
| ATOM | 970 | CD1 | LEU | 171 | 29.654 | 4.563 | −6.453 | 1.00 | 0.00 | 3A4 |
| ATOM | 971 | CD2 | LEU | 171 | 32.040 | 3.710 | −6.194 | 1.00 | 0.00 | 3A4 |
| ATOM | 972 | C | LEU | 171 | 30.998 | 1.069 | −3.277 | 1.00 | 0.00 | 3A4 |
| ATOM | 973 | O | LEU | 171 | 32.076 | 1.436 | −2.831 | 1.00 | 0.00 | 3A4 |
| ATOM | 974 | N | LYS | 172 | 30.072 | 0.510 | −2.464 | 1.00 | 0.00 | 3A4 |
| ATOM | 975 | CA | LYS | 172 | 30.261 | 0.291 | −1.041 | 1.00 | 0.00 | 3A4 |
| ATOM | 976 | CB | LYS | 172 | 28.920 | −0.132 | −0.376 | 1.00 | 0.00 | 3A4 |
| ATOM | 977 | CG | LYS | 172 | 28.909 | −0.334 | 1.158 | 1.00 | 0.00 | 3A4 |
| ATOM | 978 | CD | LYS | 172 | 29.194 | 0.939 | 1.969 | 1.00 | 0.00 | 3A4 |
| ATOM | 979 | CE | LYS | 172 | 29.078 | 0.766 | 3.488 | 1.00 | 0.00 | 3A4 |
| ATOM | 980 | NZ | LYS | 172 | 30.110 | −0.160 | 4.012 | 1.00 | 0.00 | 3A4 |
| ATOM | 981 | C | LYS | 172 | 31.330 | −0.744 | −0.748 | 1.00 | 0.00 | 3A4 |
| ATOM | 982 | O | LYS | 172 | 32.274 | −0.492 | −0.010 | 1.00 | 0.00 | 3A4 |
| ATOM | 983 | N | ASP | 173 | 31.243 | −1.937 | −1.374 | 1.00 | 0.00 | 3A4 |
| ATOM | 984 | CA | ASP | 173 | 32.134 | −3.048 | −1.103 | 1.00 | 0.00 | 3A4 |
| ATOM | 985 | CB | ASP | 173 | 31.447 | −4.365 | −1.539 | 1.00 | 0.00 | 3A4 |
| ATOM | 986 | CG | ASP | 173 | 32.095 | −5.634 | −0.940 | 1.00 | 0.00 | 3A4 |
| ATOM | 987 | OD1 | ASP | 173 | 32.129 | −5.750 | 0.315 | 1.00 | 0.00 | 3A4 |
| ATOM | 988 | OD2 | ASP | 173 | 32.559 | −6.496 | −1.735 | 1.00 | 0.00 | 3A4 |
| ATOM | 989 | C | ASP | 173 | 33.491 | −2.909 | −1.760 | 1.00 | 0.00 | 3A4 |
| ATOM | 990 | O | ASP | 173 | 34.487 | −3.361 | −1.199 | 1.00 | 0.00 | 3A4 |
| ATOM | 991 | N | VAL | 174 | 33.571 | −2.272 | −2.947 | 1.00 | 0.00 | 3A4 |
| ATOM | 992 | CA | VAL | 174 | 34.804 | −2.128 | −3.702 | 1.00 | 0.00 | 3A4 |
| ATOM | 993 | CB | VAL | 174 | 34.550 | −2.192 | −5.209 | 1.00 | 0.00 | 3A4 |
| ATOM | 994 | CG1 | VAL | 174 | 35.866 | −2.225 | −6.033 | 1.00 | 0.00 | 3A4 |
| ATOM | 995 | CG2 | VAL | 174 | 33.783 | −3.513 | −5.482 | 1.00 | 0.00 | 3A4 |
| ATOM | 996 | C | VAL | 174 | 35.560 | −0.866 | −3.321 | 1.00 | 0.00 | 3A4 |
| ATOM | 997 | O | VAL | 174 | 36.767 | −0.793 | −3.522 | 1.00 | 0.00 | 3A4 |
| ATOM | 998 | N | PHE | 175 | 34.889 | 0.162 | −2.748 | 1.00 | 0.00 | 3A4 |
| ATOM | 999 | CA | PHE | 175 | 35.513 | 1.457 | −2.585 | 1.00 | 0.00 | 3A4 |
| ATOM | 1000 | CB | PHE | 175 | 34.996 | 2.458 | −3.637 | 1.00 | 0.00 | 3A4 |
| ATOM | 1001 | CG | PHE | 175 | 35.738 | 3.789 | −3.669 | 1.00 | 0.00 | 3A4 |
| ATOM | 1002 | CD1 | PHE | 175 | 35.169 | 4.936 | −3.079 | 1.00 | 0.00 | 3A4 |
| ATOM | 1003 | CD2 | PHE | 175 | 37.019 | 3.896 | −4.239 | 1.00 | 0.00 | 3A4 |
| ATOM | 1004 | CE1 | PHE | 175 | 35.866 | 6.153 | −3.047 | 1.00 | 0.00 | 3A4 |
| ATOM | 1005 | CE2 | PHE | 175 | 37.703 | 5.120 | −4.237 | 1.00 | 0.00 | 3A4 |
| ATOM | 1006 | CZ | PHE | 175 | 37.136 | 6.244 | −3.628 | 1.00 | 0.00 | 3A4 |
| ATOM | 1007 | C | PHE | 175 | 35.362 | 1.997 | −1.219 | 1.00 | 0.00 | 3A4 |
| ATOM | 1008 | O | PHE | 175 | 36.347 | 2.184 | −0.509 | 1.00 | 0.00 | 3A4 |
| ATOM | 1009 | N | GLY | 176 | 34.105 | 2.318 | −0.836 | 1.00 | 0.00 | 3A4 |
| ATOM | 1010 | CA | GLY | 176 | 33.755 | 3.060 | 0.352 | 1.00 | 0.00 | 3A4 |
| ATOM | 1011 | C | GLY | 176 | 34.098 | 2.332 | 1.609 | 1.00 | 0.00 | 3A4 |
| ATOM | 1012 | O | GLY | 176 | 34.413 | 2.958 | 2.614 | 1.00 | 0.00 | 3A4 |
| ATOM | 1013 | N | ALA | 177 | 34.126 | 0.976 | 1.565 | 1.00 | 0.00 | 3A4 |
| ATOM | 1014 | CA | ALA | 177 | 34.485 | 0.144 | 2.688 | 1.00 | 0.00 | 3A4 |
| ATOM | 1015 | CB | ALA | 177 | 33.968 | −1.294 | 2.532 | 1.00 | 0.00 | 3A4 |
| ATOM | 1016 | C | ALA | 177 | 35.962 | 0.124 | 2.957 | 1.00 | 0.00 | 3A4 |
| ATOM | 1017 | O | ALA | 177 | 36.406 | 0.327 | 4.080 | 1.00 | 0.00 | 3A4 |
| ATOM | 1018 | N | TYR | 178 | 36.777 | −0.058 | 1.903 | 1.00 | 0.00 | 3A4 |
| ATOM | 1019 | CA | TYR | 178 | 38.203 | −0.242 | 2.016 | 1.00 | 0.00 | 3A4 |
| ATOM | 1020 | CB | TYR | 178 | 38.721 | −0.724 | 0.638 | 1.00 | 0.00 | 3A4 |
| ATOM | 1021 | CG | TYR | 178 | 38.656 | −2.235 | 0.593 | 1.00 | 0.00 | 3A4 |
| ATOM | 1022 | CD1 | TYR | 178 | 37.893 | −2.854 | −0.416 | 1.00 | 0.00 | 3A4 |
| ATOM | 1023 | CD2 | TYR | 178 | 39.236 | −3.052 | 1.584 | 1.00 | 0.00 | 3A4 |
| ATOM | 1024 | CE1 | TYR | 178 | 37.711 | −4.243 | −0.441 | 1.00 | 0.00 | 3A4 |
| ATOM | 1025 | CE2 | TYR | 178 | 39.039 | −4.442 | 1.580 | 1.00 | 0.00 | 3A4 |
| ATOM | 1026 | CZ | TYR | 178 | 38.282 | −5.040 | 0.562 | 1.00 | 0.00 | 3A4 |
| ATOM | 1027 | OH | TYR | 178 | 38.091 | −6.439 | 0.553 | 1.00 | 0.00 | 3A4 |
| ATOM | 1028 | C | TYR | 178 | 38.966 | 1.017 | 2.362 | 1.00 | 0.00 | 3A4 |
| ATOM | 1029 | O | TYR | 178 | 39.918 | 0.985 | 3.127 | 1.00 | 0.00 | 3A4 |
| ATOM | 1030 | N | SER | 179 | 38.545 | 2.180 | 1.822 | 1.00 | 0.00 | 3A4 |
| ATOM | 1031 | CA | SER | 179 | 39.171 | 3.471 | 2.018 | 1.00 | 0.00 | 3A4 |
| ATOM | 1032 | CB | SER | 179 | 38.412 | 4.545 | 1.196 | 1.00 | 0.00 | 3A4 |
| ATOM | 1033 | OG | SER | 179 | 39.108 | 5.787 | 1.074 | 1.00 | 0.00 | 3A4 |
| ATOM | 1034 | C | SER | 179 | 39.344 | 3.878 | 3.465 | 1.00 | 0.00 | 3A4 |
| ATOM | 1035 | O | SER | 179 | 40.445 | 4.039 | 3.966 | 1.00 | 0.00 | 3A4 |
| ATOM | 1036 | N | MET | 180 | 38.263 | 3.919 | 4.233 | 1.00 | 0.00 | 3A4 |
| ATOM | 1037 | CA | MET | 180 | 38.328 | 4.314 | 5.621 | 1.00 | 0.00 | 3A4 |
| ATOM | 1038 | CB | MET | 180 | 36.898 | 4.623 | 5.984 | 1.00 | 0.00 | 3A4 |
| ATOM | 1039 | CG | MET | 180 | 36.729 | 5.941 | 5.189 | 1.00 | 0.00 | 3A4 |
| ATOM | 1040 | SD | MET | 180 | 35.589 | 5.903 | 3.791 | 1.00 | 0.00 | 3A4 |
| ATOM | 1041 | CE | MET | 180 | 36.564 | 7.153 | 2.893 | 1.00 | 0.00 | 3A4 |
| ATOM | 1042 | C | MET | 180 | 38.961 | 3.325 | 6.527 | 1.00 | 0.00 | 3A4 |
| ATOM | 1043 | O | MET | 180 | 39.602 | 3.684 | 7.513 | 1.00 | 0.00 | 3A4 |
| ATOM | 1044 | N | ASP | 181 | 38.892 | 2.030 | 6.144 | 1.00 | 0.00 | 3A4 |
| ATOM | 1045 | CA | ASP | 181 | 39.455 | 1.016 | 6.971 | 1.00 | 0.00 | 3A4 |
| ATOM | 1046 | CB | ASP | 181 | 38.927 | −0.386 | 6.705 | 1.00 | 0.00 | 3A4 |
| ATOM | 1047 | CG | ASP | 181 | 38.781 | −1.148 | 5.400 | 1.00 | 0.00 | 3A4 |
| ATOM | 1048 | OD1 | ASP | 181 | 39.773 | −1.165 | 4.635 | 1.00 | 0.00 | 3A4 |
| ATOM | 1049 | OD2 | ASP | 181 | 37.728 | −1.810 | 5.195 | 1.00 | 0.00 | 3A4 |
| ATOM | 1050 | C | ASP | 181 | 40.963 | 0.976 | 6.884 | 1.00 | 0.00 | 3A4 |
| ATOM | 1051 | O | ASP | 181 | 41.663 | 0.876 | 7.886 | 1.00 | 0.00 | 3A4 |
| ATOM | 1052 | N | VAL | 182 | 41.515 | 1.163 | 5.671 | 1.00 | 0.00 | 3A4 |
| ATOM | 1053 | CA | VAL | 182 | 42.946 | 1.238 | 5.464 | 1.00 | 0.00 | 3A4 |
| ATOM | 1054 | CB | VAL | 182 | 43.279 | 1.227 | 4.010 | 1.00 | 0.00 | 3A4 |
| ATOM | 1055 | CG1 | VAL | 182 | 44.767 | 1.508 | 3.706 | 1.00 | 0.00 | 3A4 |
| ATOM | 1056 | CG2 | VAL | 182 | 42.889 | −0.181 | 3.532 | 1.00 | 0.00 | 3A4 |
| ATOM | 1057 | C | VAL | 182 | 43.593 | 2.408 | 6.151 | 1.00 | 0.00 | 3A4 |
| ATOM | 1058 | O | VAL | 182 | 44.676 | 2.298 | 6.708 | 1.00 | 0.00 | 3A4 |
| ATOM | 1059 | N | ILE | 183 | 42.877 | 3.540 | 6.210 | 1.00 | 0.00 | 3A4 |
| ATOM | 1060 | CA | ILE | 183 | 43.341 | 4.740 | 6.855 | 1.00 | 0.00 | 3A4 |
| ATOM | 1061 | CB | ILE | 183 | 42.480 | 5.913 | 6.432 | 1.00 | 0.00 | 3A4 |
| ATOM | 1062 | CG2 | ILE | 183 | 42.783 | 7.205 | 7.220 | 1.00 | 0.00 | 3A4 |
| ATOM | 1063 | CG1 | ILE | 183 | 42.712 | 6.215 | 4.926 | 1.00 | 0.00 | 3A4 |
| ATOM | 1064 | CD | ILE | 183 | 44.121 | 6.672 | 4.516 | 1.00 | 0.00 | 3A4 |
| ATOM | 1065 | C | ILE | 183 | 43.365 | 4.589 | 8.352 | 1.00 | 0.00 | 3A4 |
| ATOM | 1066 | O | ILE | 183 | 44.321 | 4.972 | 9.017 | 1.00 | 0.00 | 3A4 |
| ATOM | 1067 | N | THR | 184 | 42.323 | 3.969 | 8.947 | 1.00 | 0.00 | 3A4 |
| ATOM | 1068 | CA | THR | 184 | 42.311 | 3.780 | 10.379 | 1.00 | 0.00 | 3A4 |
| ATOM | 1069 | CB | THR | 184 | 40.896 | 3.599 | 10.968 | 1.00 | 0.00 | 3A4 |
| ATOM | 1070 | OG1 | THR | 184 | 40.842 | 3.873 | 12.369 | 1.00 | 0.00 | 3A4 |
| ATOM | 1071 | CG2 | THR | 184 | 40.253 | 2.222 | 10.686 | 1.00 | 0.00 | 3A4 |
| ATOM | 1072 | C | THR | 184 | 43.288 | 2.710 | 10.811 | 1.00 | 0.00 | 3A4 |
| ATOM | 1073 | 0 | THR | 184 | 43.934 | 2.838 | 11.842 | 1.00 | 0.00 | 3A4 |
| ATOM | 1074 | N | SER | 185 | 43.544 | 1.697 | 9.955 | 1.00 | 0.00 | 3A4 |
| ATOM | 1075 | CA | SER | 185 | 44.571 | 0.701 | 10.179 | 1.00 | 0.00 | 3A4 |
| ATOM | 1076 | CB | SER | 185 | 44.486 | −0.452 | 9.173 | 1.00 | 0.00 | 3A4 |
| ATOM | 1077 | OG | SER | 185 | 43.278 | −1.178 | 9.354 | 1.00 | 0.00 | 3A4 |
| ATOM | 1078 | C | SER | 185 | 45.975 | 1.270 | 10.157 | 1.00 | 0.00 | 3A4 |
| ATOM | 1079 | O | SER | 185 | 46.793 | 0.962 | 11.011 | 1.00 | 0.00 | 3A4 |
| ATOM | 1080 | N | THR | 186 | 46.255 | 2.211 | 9.238 | 1.00 | 0.00 | 3A4 |
| ATOM | 1081 | CA | THR | 186 | 47.523 | 2.905 | 9.158 | 1.00 | 0.00 | 3A4 |
| ATOM | 1082 | CB | THR | 186 | 47.674 | 3.626 | 7.845 | 1.00 | 0.00 | 3A4 |
| ATOM | 1083 | OG1 | THR | 186 | 46.613 | 4.495 | 7.478 | 1.00 | 0.00 | 3A4 |
| ATOM | 1084 | CG2 | THR | 186 | 47.869 | 2.559 | 6.746 | 1.00 | 0.00 | 3A4 |
| ATOM | 1085 | C | THR | 186 | 47.770 | 3.895 | 10.256 | 1.00 | 0.00 | 3A4 |
| ATOM | 1086 | O | THR | 186 | 48.906 | 4.171 | 10.627 | 1.00 | 0.00 | 3A4 |
| ATOM | 1087 | N | SER | 187 | 46.676 | 4.433 | 10.825 | 1.00 | 0.00 | 3A4 |
| ATOM | 1088 | CA | SER | 187 | 46.674 | 5.311 | 11.961 | 1.00 | 0.00 | 3A4 |
| ATOM | 1089 | CB | SER | 187 | 45.260 | 5.983 | 12.078 | 1.00 | 0.00 | 3A4 |
| ATOM | 1090 | OG | SER | 187 | 45.037 | 6.742 | 13.262 | 1.00 | 0.00 | 3A4 |
| ATOM | 1091 | C | SER | 187 | 47.012 | 4.556 | 13.221 | 1.00 | 0.00 | 3A4 |
| ATOM | 1092 | O | SER | 187 | 47.753 | 5.052 | 14.047 | 1.00 | 0.00 | 3A4 |
| ATOM | 1093 | N | PHE | 188 | 46.490 | 3.324 | 13.352 | 1.00 | 0.00 | 3A4 |
| ATOM | 1094 | CA | PHE | 188 | 46.634 | 2.477 | 14.511 | 1.00 | 0.00 | 3A4 |
| ATOM | 1095 | CB | PHE | 188 | 45.408 | 1.520 | 14.609 | 1.00 | 0.00 | 3A4 |
| ATOM | 1096 | CG | PHE | 188 | 44.440 | 2.221 | 15.507 | 1.00 | 0.00 | 3A4 |
| ATOM | 1097 | CD1 | PHE | 108 | 43.828 | 3.428 | 15.112 | 1.00 | 0.00 | 3A4 |
| ATOM | 1098 | CD2 | PHE | 188 | 44.425 | 1.853 | 16.860 | 1.00 | 0.00 | 3A4 |
| ATOM | 1099 | CE1 | PHE | 188 | 43.309 | 4.298 | 16.068 | 1.00 | 0.00 | 3A4 |
| ATOM | 1100 | CE2 | PHE | 188 | 43.917 | 2.727 | 17.815 | 1.00 | 0.00 | 3A4 |
| ATOM | 1101 | CZ | PHE | 188 | 43.370 | 3.945 | 17.418 | 1.00 | 0.00 | 3A4 |
| ATOM | 1102 | C | PHE | 188 | 47.882 | 1.665 | 14.537 | 1.00 | 0.00 | 3A4 |
| ATOM | 1103 | O | PHE | 188 | 48.386 | 1.332 | 15.605 | 1.00 | 0.00 | 3A4 |
| ATOM | 1104 | N | GLY | 189 | 48.414 | 1.335 | 13.350 | 1.00 | 0.00 | 3A4 |
| ATOM | 1105 | CA | GLY | 189 | 49.610 | 0.550 | 13.208 | 1.00 | 0.00 | 3A4 |
| ATOM | 1106 | C | GLY | 189 | 49.281 | −0.923 | 13.101 | 1.00 | 0.00 | 3A4 |
| ATOM | 1107 | O | GLY | 189 | 50.025 | −1.788 | 13.555 | 1.00 | 0.00 | 3A4 |
| ATOM | 1108 | N | VAL | 190 | 48.147 | −1.240 | 12.428 | 1.00 | 0.00 | 3A4 |
| ATOM | 1109 | CA | VAL | 190 | 47.745 | −2.576 | 12.011 | 1.00 | 0.00 | 3A4 |
| ATOM | 1110 | CB | VAL | 190 | 46.239 | −2.768 | 11.929 | 1.00 | 0.00 | 3A4 |
| ATOM | 1111 | CG1 | VAL | 190 | 45.823 | −4.253 | 11.759 | 1.00 | 0.00 | 3A4 |
| ATOM | 1112 | CG2 | VAL | 190 | 45.621 | −2.217 | 13.235 | 1.00 | 0.00 | 3A4 |
| ATOM | 1113 | C | VAL | 190 | 48.306 | −2.843 | 10.635 | 1.00 | 0.00 | 3A4 |
| ATOM | 1114 | O | VAL | 190 | 48.419 | −1.948 | 9.801 | 1.00 | 0.00 | 3A4 |
| ATOM | 1115 | N | ASN | 191 | 48.675 | −4.105 | 10.372 | 1.00 | 0.00 | 3A4 |
| ATOM | 1116 | CA | ASN | 191 | 49.636 | −4.477 | 9.377 | 1.00 | 0.00 | 3A4 |
| ATOM | 1117 | CB | ASN | 191 | 49.869 | −5.997 | 9.517 | 1.00 | 0.00 | 3A4 |
| ATOM | 1118 | CG | ASN | 191 | 51.211 | −6.502 | 8.934 | 1.00 | 0.00 | 3A4 |
| ATOM | 1119 | OD1 | ASN | 191 | 52.277 | −6.160 | 9.445 | 1.00 | 0.00 | 3A4 |
| ATOM | 1120 | ND2 | ASN | 191 | 51.156 | −7.322 | 7.848 | 1.00 | 0.00 | 3A4 |
| ATOM | 1121 | C | ASN | 191 | 49.394 | −4.146 | 7.970 | 1.00 | 0.00 | 3A4 |
| ATOM | 1122 | O | ASN | 191 | 48.268 | −4.363 | 7.523 | 1.00 | 0.00 | 3A4 |
| ATOM | 1123 | N | ILE | 192 | 50.505 | −3.532 | 7.398 | 1.00 | 0.00 | 3A4 |
| ATOM | 1124 | CA | ILE | 192 | 50.796 | −3.011 | 6.064 | 1.00 | 0.00 | 3A4 |
| ATOM | 1125 | CB | ILE | 192 | 49.572 | −3.045 | 5.191 | 1.00 | 0.00 | 3A4 |
| ATOM | 1126 | CG2 | ILE | 192 | 48.539 | −2.012 | 5.843 | 1.00 | 0.00 | 3A4 |
| ATOM | 1127 | CG1 | ILE | 192 | 49.850 | −3.205 | 3.643 | 1.00 | 0.00 | 3A4 |
| ATOM | 1128 | CD | ILE | 192 | 50.855 | −4.296 | 3.249 | 1.00 | 0.00 | 3A4 |
| ATOM | 1129 | C | ILE | 192 | 51.332 | −1.562 | 6.101 | 1.00 | 0.00 | 3A4 |
| ATOM | 1130 | O | ILE | 192 | 51.714 | −1.049 | 7.152 | 1.00 | 0.00 | 3A4 |
| ATOM | 1131 | N | ASP | 193 | 51.273 | −0.876 | 4.912 | 1.00 | 0.00 | 3A4 |
| ATOM | 1132 | CA | ASP | 193 | 51.280 | 0.551 | 4.649 | 1.00 | 0.00 | 3A4 |
| ATOM | 1133 | CB | ASP | 193 | 52.500 | 0.950 | 3.755 | 1.00 | 0.00 | 3A4 |
| ATOM | 1134 | CG | ASP | 193 | 52.747 | 2.473 | 3.713 | 1.00 | 0.00 | 3A4 |
| ATOM | 1135 | OD1 | ASP | 193 | 52.690 | 3.055 | 2.596 | 1.00 | 0.00 | 3A4 |
| ATOM | 1136 | OD2 | ASP | 193 | 52.993 | 3.065 | 4.798 | 1.00 | 0.00 | 3A4 |
| ATOM | 1137 | C | ASP | 193 | 49.942 | 0.941 | 3.982 | 1.00 | 0.00 | 3A4 |
| ATOM | 1138 | O | ASP | 193 | 49.555 | 2.107 | 4.042 | 1.00 | 0.00 | 3A4 |
| ATOM | 1139 | N | SER | 194 | 49.199 | −0.025 | 3.351 | 1.00 | 0.00 | 3A4 |
| ATOM | 1140 | CA | SER | 194 | 47.801 | 0.108 | 2.935 | 1.00 | 0.00 | 3A4 |
| ATOM | 1141 | CB | SER | 194 | 47.674 | 0.859 | 1.564 | 1.00 | 0.00 | 3A4 |
| ATOM | 1142 | OG | SER | 194 | 48.568 | 0.364 | 0.570 | 1.00 | 0.00 | 3A4 |
| ATOM | 1143 | C | SER | 194 | 47.072 | −1.263 | 2.877 | 1.00 | 0.00 | 3A4 |
| ATOM | 1144 | O | SER | 194 | 47.201 | −1.949 | 1.864 | 1.00 | 0.00 | 3A4 |
| ATOM | 1145 | N | LEU | 195 | 46.300 | −1.682 | 3.968 | 1.00 | 0.00 | 3A4 |
| ATOM | 1146 | CA | LEU | 195 | 45.506 | −2.928 | 4.205 | 1.00 | 0.00 | 3A4 |
| ATOM | 1147 | CB | LEU | 195 | 44.616 | −3.155 | 2.908 | 1.00 | 0.00 | 3A4 |
| ATOM | 1148 | CG | LEU | 195 | 43.358 | −4.046 | 2.893 | 1.00 | 0.00 | 3A4 |
| ATOM | 1149 | CD1 | LEU | 195 | 42.566 | −3.731 | 1.614 | 1.00 | 0.00 | 3A4 |
| ATOM | 1150 | CD2 | LEU | 195 | 43.591 | −5.569 | 2.963 | 1.00 | 0.00 | 3A4 |
| ATOM | 1151 | C | LEU | 195 | 46.313 | −4.231 | 4.384 | 1.00 | 0.00 | 3A4 |
| ATOM | 1152 | O | LEU | 195 | 46.823 | −4.525 | 3.303 | 1.00 | 0.00 | 3A4 |
| ATOM | 1153 | N | ASN | 196 | 46.462 | −5.133 | 5.522 | 1.00 | 0.00 | 3A4 |
| ATOM | 1154 | CA | ASN | 196 | 46.660 | −6.447 | 5.007 | 1.00 | 0.00 | 3A4 |
| ATOM | 1155 | CB | ASN | 196 | 48.141 | −6.561 | 4.645 | 1.00 | 0.00 | 3A4 |
| ATOM | 1156 | CG | ASN | 196 | 48.532 | −7.677 | 3.641 | 1.00 | 0.00 | 3A4 |
| ATOM | 1157 | OD1 | ASN | 196 | 49.287 | −8.581 | 3.992 | 1.00 | 0.00 | 3A4 |
| ATOM | 1158 | ND2 | ASN | 196 | 48.039 | −7.601 | 2.374 | 1.00 | 0.00 | 3A4 |
| ATOM | 1159 | C | ASN | 196 | 46.000 | −7.578 | 5.603 | 1.00 | 0.00 | 3A4 |
| ATOM | 1160 | O | ASN | 196 | 44.826 | −7.879 | 5.338 | 1.00 | 0.00 | 3A4 |
| ATOM | 1161 | N | ASN | 197 | 46.757 | −8.297 | 6.407 | 1.00 | 0.00 | 3A4 |
| ATOM | 1162 | CA | ASN | 197 | 46.317 | −9.599 | 6.859 | 1.00 | 0.00 | 3A4 |
| ATOM | 1163 | CB | ASN | 197 | 47.456 | −10.511 | 7.443 | 1.00 | 0.00 | 3A4 |
| ATOM | 1164 | CG | ASN | 197 | 48.572 | −10.689 | 6.405 | 1.00 | 0.00 | 3A4 |
| ATOM | 1165 | OD1 | ASN | 197 | 48.334 | −11.249 | 5.336 | 1.00 | 0.00 | 3A4 |
| ATOM | 1166 | ND2 | ASN | 197 | 49.810 | −10.219 | 6.717 | 1.00 | 0.00 | 3A4 |
| ATOM | 1167 | C | ASN | 197 | 45.160 | −9.556 | 7.804 | 1.00 | 0.00 | 3A4 |
| ATOM | 1168 | O | ASN | 197 | 44.307 | −10.439 | 7.761 | 1.00 | 0.00 | 3A4 |
| ATOM | 1169 | N | PRO | 198 | 45.075 | −8.496 | 8.595 | 1.00 | 0.00 | 3A4 |
| ATOM | 1170 | CA | PRO | 198 | 44.013 | −8.420 | 9.555 | 1.00 | 0.00 | 3A4 |
| ATOM | 1171 | CD | PRO | 198 | 46.277 | −7.818 | 9.154 | 1.00 | 0.00 | 3A4 |
| ATOM | 1172 | CB | PRO | 198 | 44.652 | −7.631 | 10.641 | 1.00 | 0.00 | 3A4 |
| ATOM | 1173 | CG | PRO | 198 | 46.123 | −7.908 | 10.628 | 1.00 | 0.00 | 3A4 |
| ATOM | 1174 | C | PRO | 198 | 42.754 | −7.786 | 9.085 | 1.00 | 0.00 | 3A4 |
| ATOM | 1175 | O | PRO | 198 | 41.799 | −7.743 | 9.865 | 1.00 | 0.00 | 3A4 |
| ATOM | 1176 | N | GLN | 199 | 42.701 | −7.279 | 7.830 | 1.00 | 0.00 | 3A4 |
| ATOM | 1177 | CA | GLN | 199 | 41.636 | −6.407 | 7.407 | 1.00 | 0.00 | 3A4 |
| ATOM | 1178 | CB | GLN | 199 | 41.900 | −5.668 | 6.066 | 1.00 | 0.00 | 3A4 |
| ATOM | 1179 | CG | GLN | 199 | 41.001 | −4.419 | 5.844 | 1.00 | 0.00 | 3A4 |
| ATOM | 1180 | CD | GLN | 199 | 41.471 | −3.266 | 6.742 | 1.00 | 0.00 | 3A4 |
| ATOM | 1181 | OE1 | GLN | 199 | 42.353 | −2.503 | 6.348 | 1.00 | 0.00 | 3A4 |
| ATOM | 1182 | NE2 | GLN | 199 | 40.885 | −3.125 | 7.963 | 1.00 | 0.00 | 3A4 |
| ATOM | 1183 | C | GLN | 199 | 40.319 | −7.071 | 7.338 | 1.00 | 0.00 | 3A4 |
| ATOM | 1184 | O | GLN | 199 | 39.374 | −6.533 | 7.889 | 1.00 | 0.00 | 3A4 |
| ATOM | 1185 | N | ASP | 200 | 40.193 | −8.262 | 6.717 | 1.00 | 0.00 | 3A4 |
| ATOM | 1186 | CA | ASP | 200 | 38.939 | −8.983 | 6.597 | 1.00 | 0.00 | 3A4 |
| ATOM | 1187 | CB | ASP | 200 | 39.049 | −10.138 | 5.538 | 1.00 | 0.00 | 3A4 |
| ATOM | 1188 | CG | ASP | 200 | 37.683 | −10.693 | 5.079 | 1.00 | 0.00 | 3A4 |
| ATOM | 1189 | OD1 | ASP | 200 | 37.459 | −11.923 | 5.238 | 1.00 | 0.00 | 3A4 |
| ATOM | 1190 | OD2 | ASP | 200 | 36.855 | −9.893 | 4.566 | 1.00 | 0.00 | 3A4 |
| ATOM | 1191 | C | ASP | 200 | 38.347 | −9.432 | 7.927 | 1.00 | 0.00 | 3A4 |
| ATOM | 1192 | O | ASP | 200 | 37.137 | −9.315 | 8.099 | 1.00 | 0.00 | 3A4 |
| ATOM | 1193 | N | PRO | 201 | 39.155 | −9.847 | 8.917 | 1.00 | 0.00 | 3A4 |
| ATOM | 1194 | CA | PRO | 201 | 38.699 | −10.088 | 10.270 | 1.00 | 0.00 | 3A4 |
| ATOM | 1195 | CD | PRO | 201 | 40.343 | −10.678 | 8.654 | 1.00 | 0.00 | 3A4 |
| ATOM | 1196 | CB | PRO | 201 | 39.936 | −10.637 | 11.012 | 1.00 | 0.00 | 3A4 |
| ATOM | 1197 | CG | PRO | 201 | 40.633 | −11.466 | 9.938 | 1.00 | 0.00 | 3A4 |
| ATOM | 1198 | C | PRO | 201 | 38.128 | −8.916 | 10.997 | 1.00 | 0.00 | 3A4 |
| ATOM | 1199 | O | PRO | 201 | 37.076 | −9.052 | 11.615 | 1.00 | 0.00 | 3A4 |
| ATOM | 1200 | N | PHE | 202 | 38.762 | −7.733 | 10.872 | 1.00 | 0.00 | 3A4 |
| ATOM | 1201 | CA | PHE | 202 | 38.319 | −6.521 | 11.530 | 1.00 | 0.00 | 3A4 |
| ATOM | 1202 | CB | PHE | 202 | 39.462 | −5.473 | 11.494 | 1.00 | 0.00 | 3A4 |
| ATOM | 1203 | CG | PHE | 202 | 40.009 | −5.259 | 12.888 | 1.00 | 0.00 | 3A4 |
| ATOM | 1204 | CD1 | PHE | 202 | 40.365 | −6.366 | 13.691 | 1.00 | 0.00 | 3A4 |
| ATOM | 1205 | CD2 | PHE | 202 | 40.293 | −3.968 | 13.372 | 1.00 | 0.00 | 3A4 |
| ATOM | 1206 | CE1 | PHE | 202 | 40.856 | −6.184 | 14.987 | 1.00 | 0.00 | 3A4 |
| ATOM | 1207 | CE2 | PHE | 202 | 40.855 | −3.790 | 14.647 | 1.00 | 0.00 | 3A4 |
| ATOM | 1208 | CZ | PHE | 202 | 41.107 | −4.894 | 15.465 | 1.00 | 0.00 | 3A4 |
| ATOM | 1209 | C | PHE | 202 | 37.080 | −5.964 | 10.887 | 1.00 | 0.00 | 3A4 |
| ATOM | 1210 | O | PHE | 202 | 36.208 | −5.444 | 11.573 | 1.00 | 0.00 | 3A4 |
| ATOM | 1211 | N | VAL | 203 | 36.928 | −6.145 | 9.554 | 1.00 | 0.00 | 3A4 |
| ATOM | 1212 | CA | VAL | 203 | 35.752 | −5.762 | 8.799 | 1.00 | 0.00 | 3A4 |
| ATOM | 1213 | CB | VAL | 203 | 36.015 | −5.808 | 7.287 | 1.00 | 0.00 | 3A4 |
| ATOM | 1214 | CG1 | VAL | 203 | 34.738 | −5.677 | 6.411 | 1.00 | 0.00 | 3A4 |
| ATOM | 1215 | CG2 | VAL | 203 | 36.983 | −4.648 | 6.954 | 1.00 | 0.00 | 3A4 |
| ATOM | 1216 | C | VAL | 203 | 34.545 | −6.596 | 9.199 | 1.00 | 0.00 | 3A4 |
| ATOM | 1217 | O | VAL | 203 | 33.451 | −6.068 | 9.364 | 1.00 | 0.00 | 3A4 |
| ATOM | 1218 | N | GLU | 204 | 34.736 | −7.908 | 9.475 | 1.00 | 0.00 | 3A4 |
| ATOM | 1219 | CA | GLU | 204 | 33.684 | −8.782 | 9.967 | 1.00 | 0.00 | 3A4 |
| ATOM | 1220 | CB | GLU | 204 | 34.114 | −10.262 | 9.912 | 1.00 | 0.00 | 3A4 |
| ATOM | 1221 | CG | GLU | 204 | 34.199 | −10.797 | 8.472 | 1.00 | 0.00 | 3A4 |
| ATOM | 1222 | CD | GLU | 204 | 34.793 | −12.210 | 8.489 | 1.00 | 0.00 | 3A4 |
| ATOM | 1223 | OE1 | GLU | 204 | 35.910 | −12.394 | 7.934 | 1.00 | 0.00 | 3A4 |
| ATOM | 1224 | OE2 | GLU | 204 | 34.137 | −13.123 | 9.059 | 1.00 | 0.00 | 3A4 |
| ATOM | 1225 | C | GLU | 204 | 33.230 | −8.440 | 11.373 | 1.00 | 0.00 | 3A4 |
| ATOM | 1226 | O | GLU | 204 | 32.042 | −8.449 | 11.666 | 1.00 | 0.00 | 3A4 |
| ATOM | 1227 | N | ASN | 205 | 34.176 | −8.057 | 12.259 | 1.00 | 0.00 | 3A4 |
| ATOM | 1228 | CA | ASN | 205 | 33.914 | −7.660 | 13.628 | 1.00 | 0.00 | 3A4 |
| ATOM | 1229 | CB | ASN | 205 | 35.248 | −7.438 | 14.383 | 1.00 | 0.00 | 3A4 |
| ATOM | 1230 | CG | ASN | 205 | 36.015 | −8.751 | 14.628 | 1.00 | 0.00 | 3A4 |
| ATOM | 1231 | OD1 | ASN | 205 | 37.233 | −8.776 | 14.460 | 1.00 | 0.00 | 3A4 |
| ATOM | 1232 | ND2 | ASN | 205 | 35.329 | −9.842 | 15.069 | 1.00 | 0.00 | 3A4 |
| ATOM | 1233 | C | ASN | 205 | 33.087 | −6.395 | 13.736 | 1.00 | 0.00 | 3A4 |
| ATOM | 1234 | O | ASN | 205 | 32.115 | −6.344 | 14.487 | 1.00 | 0.00 | 3A4 |
| ATOM | 1235 | N | THR | 206 | 33.414 | −5.376 | 12.903 | 1.00 | 0.00 | 3A4 |
| ATOM | 1236 | CA | THR | 206 | 32.762 | −4.077 | 12.899 | 1.00 | 0.00 | 3A4 |
| ATOM | 1237 | CB | THR | 206 | 33.525 | −3.007 | 12.102 | 1.00 | 0.00 | 3A4 |
| ATOM | 1238 | OG1 | THR | 206 | 34.406 | −3.549 | 11.139 | 1.00 | 0.00 | 3A4 |
| ATOM | 1239 | CG2 | THR | 206 | 34.381 | −2.246 | 13.141 | 1.00 | 0.00 | 3A4 |
| ATOM | 1240 | C | THR | 206 | 31.364 | −4.178 | 12.322 | 1.00 | 0.00 | 3A4 |
| ATOM | 1241 | O | THR | 206 | 30.435 | −3.581 | 12.843 | 1.00 | 0.00 | 3A4 |
| ATOM | 1242 | N | LYS | 207 | 31.178 | −5.009 | 11.279 | 1.00 | 0.00 | 3A4 |
| ATOM | 1243 | CA | LYS | 207 | 29.905 | −5.266 | 10.645 | 1.00 | 0.00 | 3A4 |
| ATOM | 1244 | CB | LYS | 207 | 30.122 | −6.061 | 9.349 | 1.00 | 0.00 | 3A4 |
| ATOM | 1245 | CG | LYS | 207 | 28.927 | −6.211 | 8.393 | 1.00 | 0.00 | 3A4 |
| ATOM | 1246 | CD | LYS | 207 | 29.306 | −6.802 | 7.027 | 1.00 | 0.00 | 3A4 |
| ATOM | 1247 | CE | LYS | 207 | 29.832 | −8.246 | 7.088 | 1.00 | 0.00 | 3A4 |
| ATOM | 1248 | NZ | LYS | 207 | 30.150 | −8.750 | 5.731 | 1.00 | 0.00 | 3A4 |
| ATOM | 1249 | C | LYS | 207 | 28.938 | −6.023 | 11.523 | 1.00 | 0.00 | 3A4 |
| ATOM | 1250 | O | LYS | 207 | 27.753 | −5.736 | 11.533 | 1.00 | 0.00 | 3A4 |
| ATOM | 1251 | N | LYS | 208 | 29.441 | −6.958 | 12.359 | 1.00 | 0.00 | 3A4 |
| ATOM | 1252 | CA | LYS | 208 | 28.646 | −7.727 | 13.293 | 1.00 | 0.00 | 3A4 |
| ATOM | 1253 | CB | LYS | 208 | 29.443 | −8.920 | 13.865 | 1.00 | 0.00 | 3A4 |
| ATOM | 1254 | CG | LYS | 208 | 28.627 | −9.993 | 14.610 | 1.00 | 0.00 | 3A4 |
| ATOM | 1255 | CD | LYS | 208 | 29.414 | −11.248 | 15.031 | 1.00 | 0.00 | 3A4 |
| ATOM | 1256 | CE | LYS | 208 | 29.894 | −12.177 | 13.900 | 1.00 | 0.00 | 3A4 |
| ATOM | 1257 | NZ | LYS | 208 | 31.125 | −11.677 | 13.236 | 1.00 | 0.00 | 3A4 |
| ATOM | 1258 | C | LYS | 208 | 28.094 | −6.905 | 14.430 | 1.00 | 0.00 | 3A4 |
| ATOM | 1259 | O | LYS | 208 | 26.931 | −7.047 | 14.780 | 1.00 | 0.00 | 3A4 |
| ATOM | 1260 | N | LEU | 209 | 28.886 | −5.983 | 15.022 | 1.00 | 0.00 | 3A4 |
| ATOM | 1261 | CA | LEU | 209 | 28.454 | −5.149 | 16.133 | 1.00 | 0.00 | 3A4 |
| ATOM | 1262 | CB | LEU | 209 | 29.656 | −4.415 | 16.784 | 1.00 | 0.00 | 3A4 |
| ATOM | 1263 | CG | LEU | 209 | 29.353 | −3.397 | 17.945 | 1.00 | 0.00 | 3A4 |
| ATOM | 1264 | CD1 | LEU | 209 | 28.592 | −4.009 | 19.146 | 1.00 | 0.00 | 3A4 |
| ATOM | 1265 | CD2 | LEU | 209 | 30.596 | −2.606 | 18.399 | 1.00 | 0.00 | 3A4 |
| ATOM | 1266 | C | LEU | 209 | 27.416 | −4.112 | 15.737 | 1.00 | 0.00 | 3A4 |
| ATOM | 1267 | O | LEU | 209 | 26.515 | −3.793 | 16.507 | 1.00 | 0.00 | 3A4 |
| ATOM | 1268 | N | LEU | 210 | 27.527 | −3.561 | 14.510 | 1.00 | 0.00 | 3A4 |
| ATOM | 1269 | CA | LEU | 210 | 26.707 | −2.449 | 14.080 | 1.00 | 0.00 | 3A4 |
| ATOM | 1270 | CB | LEU | 210 | 27.559 | −1.343 | 13.418 | 1.00 | 0.00 | 3A4 |
| ATOM | 1271 | CG | LEU | 210 | 28.684 | −0.850 | 14.384 | 1.00 | 0.00 | 3A4 |
| ATOM | 1272 | CD1 | LEU | 210 | 29.655 | 0.113 | 13.699 | 1.00 | 0.00 | 3A4 |
| ATOM | 1273 | CD2 | LEU | 210 | 28.176 | −0.257 | 15.717 | 1.00 | 0.00 | 3A4 |
| ATOM | 1274 | C | LEU | 210 | 25.611 | −2.862 | 13.145 | 1.00 | 0.00 | 3A4 |
| ATOM | 1275 | O | LEU | 210 | 24.465 | −2.454 | 13.335 | 1.00 | 0.00 | 3A4 |
| ATOM | 1276 | N | ARG | 211 | 25.948 | −3.571 | 12.035 | 1.00 | 0.00 | 3A4 |
| ATOM | 1277 | CA | ARG | 211 | 25.146 | −3.650 | 10.816 | 1.00 | 0.00 | 3A4 |
| ATOM | 1278 | CB | ARG | 211 | 26.033 | −3.592 | 9.539 | 1.00 | 0.00 | 3A4 |
| ATOM | 1279 | CG | ARG | 211 | 26.927 | −2.337 | 9.503 | 1.00 | 0.00 | 3A4 |
| ATOM | 1280 | CD | ARG | 211 | 27.841 | −2.220 | 8.274 | 1.00 | 0.00 | 3A4 |
| ATOM | 1281 | NE | ARG | 211 | 27.005 | −2.107 | 7.024 | 1.00 | 0.00 | 3A4 |
| ATOM | 1282 | CZ | ARG | 211 | 26.814 | −0.939 | 6.329 | 1.00 | 0.00 | 3A4 |
| ATOM | 1283 | NH1 | ARG | 211 | 26.014 | −0.951 | 5.223 | 1.00 | 0.00 | 3A4 |
| ATOM | 1284 | NH2 | ARG | 211 | 27.397 | 0.236 | 6.711 | 1.00 | 0.00 | 3A4 |
| ATOM | 1285 | C | ARG | 211 | 24.236 | −4.864 | 10.757 | 1.00 | 0.00 | 3A4 |
| ATOM | 1286 | O | ARG | 211 | 23.275 | −4.868 | 9.989 | 1.00 | 0.00 | 3A4 |
| ATOM | 1287 | N | PHE | 212 | 24.496 | −5.910 | 11.589 | 1.00 | 0.00 | 3A4 |
| ATOM | 1288 | CA | PHE | 212 | 23.555 | −6.992 | 11.826 | 1.00 | 0.00 | 3A4 |
| ATOM | 1289 | CB | PHE | 212 | 23.730 | −8.273 | 10.930 | 1.00 | 0.00 | 3A4 |
| ATOM | 1290 | CG | PHE | 212 | 25.061 | −8.998 | 10.978 | 1.00 | 0.00 | 3A4 |
| ATOM | 1291 | CD1 | PHE | 212 | 25.170 | −10.224 | 11.667 | 1.00 | 0.00 | 3A4 |
| ATOM | 1292 | CD2 | PHE | 212 | 26.182 | −8.527 | 10.268 | 1.00 | 0.00 | 3A4 |
| ATOM | 1293 | CE1 | PHE | 212 | 26.367 | −10.954 | 11.660 | 1.00 | 0.00 | 3A4 |
| ATOM | 1294 | CE2 | PHE | 212 | 27.380 | −9.259 | 10.254 | 1.00 | 0.00 | 3A4 |
| ATOM | 1295 | CZ | PHE | 212 | 27.474 | −10.469 | 10.952 | 1.00 | 0.00 | 3A4 |
| ATOM | 1296 | C | PHE | 212 | 23.608 | −7.246 | 13.314 | 1.00 | 0.00 | 3A4 |
| ATOM | 1297 | O | PHE | 212 | 24.292 | −8.143 | 13.802 | 1.00 | 0.00 | 3A4 |
| ATOM | 1298 | N | ASP | 213 | 22.863 | −6.398 | 14.068 | 1.00 | 0.00 | 3A4 |
| ATOM | 1299 | CA | ASP | 213 | 22.843 | −6.356 | 15.513 | 1.00 | 0.00 | 3A4 |
| ATOM | 1300 | CB | ASP | 213 | 23.642 | −5.131 | 16.046 | 1.00 | 0.00 | 3A4 |
| ATOM | 1301 | CG | ASP | 213 | 23.916 | −5.215 | 17.558 | 1.00 | 0.00 | 3A4 |
| ATOM | 1302 | OD1 | ASP | 213 | 24.608 | −6.179 | 17.982 | 1.00 | 0.00 | 3A4 |
| ATOM | 1303 | OD2 | ASP | 213 | 23.444 | −4.312 | 18.300 | 1.00 | 0.00 | 3A4 |
| ATOM | 1304 | C | ASP | 213 | 21.391 | −6.281 | 15.902 | 1.00 | 0.00 | 3A4 |
| ATOM | 1305 | O | ASP | 213 | 20.714 | −5.286 | 15.648 | 1.00 | 0.00 | 3A4 |
| ATOM | 1306 | N | PHE | 214 | 20.894 | −7.371 | 16.533 | 1.00 | 0.00 | 3A4 |
| ATOM | 1307 | CA | PHE | 214 | 19.516 | −7.516 | 16.941 | 1.00 | 0.00 | 3A4 |
| ATOM | 1308 | CB | PHE | 214 | 18.526 | −7.900 | 15.776 | 1.00 | 0.00 | 3A4 |
| ATOM | 1309 | CG | PHE | 214 | 19.127 | −8.826 | 14.734 | 1.00 | 0.00 | 3A4 |
| ATOM | 1310 | CD1 | PHE | 214 | 19.610 | −8.306 | 13.515 | 1.00 | 0.00 | 3A4 |
| ATOM | 1311 | CD2 | PHE | 214 | 19.216 | −10.215 | 14.950 | 1.00 | 0.00 | 3A4 |
| ATOM | 1312 | CE1 | PHE | 214 | 20.186 | −9.143 | 12.550 | 1.00 | 0.00 | 3A4 |
| ATOM | 1313 | CE2 | PHE | 214 | 19.793 | −11.058 | 13.987 | 1.00 | 0.00 | 3A4 |
| ATOM | 1314 | CZ | PHE | 214 | 20.280 | −10.520 | 12.788 | 1.00 | 0.00 | 3A4 |
| ATOM | 1315 | C | PHE | 214 | 19.513 | −8.554 | 18.034 | 1.00 | 0.00 | 3A4 |
| ATOM | 1316 | O | PHE | 214 | 20.344 | −9.460 | 18.058 | 1.00 | 0.00 | 3A4 |
| ATOM | 1317 | N | LEU | 215 | 18.527 | −8.428 | 18.956 | 1.00 | 0.00 | 3A4 |
| ATOM | 1318 | CA | LEU | 215 | 18.216 | −9.384 | 19.995 | 1.00 | 0.00 | 3A4 |
| ATOM | 1319 | CB | LEU | 215 | 18.914 | −9.072 | 21.353 | 1.00 | 0.00 | 3A4 |
| ATOM | 1320 | CG | LEU | 215 | 18.685 | −10.089 | 22.505 | 1.00 | 0.00 | 3A4 |
| ATOM | 1321 | CD1 | LEU | 215 | 19.075 | −11.533 | 22.128 | 1.00 | 0.00 | 3A4 |
| ATOM | 1322 | CD2 | LEU | 215 | 19.419 | −9.639 | 23.783 | 1.00 | 0.00 | 3A4 |
| ATOM | 1323 | C | LEU | 215 | 16.716 | −9.298 | 20.078 | 1.00 | 0.00 | 3A4 |
| ATOM | 1324 | O | LEU | 215 | 16.151 | −8.667 | 20.970 | 1.00 | 0.00 | 3A4 |
| ATOM | 1325 | N | ASP | 216 | 16.046 | −9.926 | 19.073 | 1.00 | 0.00 | 3A4 |
| ATOM | 1326 | CA | ASP | 216 | 14.627 | −9.830 | 18.781 | 1.00 | 0.00 | 3A4 |
| ATOM | 1327 | CB | ASP | 216 | 14.369 | −9.623 | 17.242 | 1.00 | 0.00 | 3A4 |
| ATOM | 1328 | CG | ASP | 216 | 14.915 | −10.746 | 16.328 | 1.00 | 0.00 | 3A4 |
| ATOM | 1329 | OD1 | ASP | 216 | 16.161 | −10.914 | 16.241 | 1.00 | 0.00 | 3A4 |
| ATOM | 1330 | OD2 | ASP | 216 | 14.075 | −11.445 | 15.700 | 1.00 | 0.00 | 3A4 |
| ATOM | 1331 | C | ASP | 216 | 13.914 | −11.069 | 19.316 | 1.00 | 0.00 | 3A4 |
| ATOM | 1332 | O | ASP | 216 | 14.578 | −12.088 | 19.506 | 1.00 | 0.00 | 3A4 |
| ATOM | 1333 | N | PRO | 217 | 12.576 | −11.062 | 19.553 | 1.00 | 0.00 | 3A4 |
| ATOM | 1334 | CA | PRO | 217 | 11.794 | −12.227 | 19.974 | 1.00 | 0.00 | 3A4 |
| ATOM | 1335 | CD | PRO | 217 | 11.814 | −9.819 | 19.695 | 1.00 | 0.00 | 3A4 |
| ATOM | 1336 | CB | PRO | 217 | 10.525 | −11.614 | 20.611 | 1.00 | 0.00 | 3A4 |
| ATOM | 1337 | CG | PRO | 217 | 10.367 | −10.231 | 19.970 | 1.00 | 0.00 | 3A4 |
| ATOM | 1338 | C | PRO | 217 | 11.504 | −13.148 | 18.786 | 1.00 | 0.00 | 3A4 |
| ATOM | 1339 | O | PRO | 217 | 12.309 | −14.043 | 18.527 | 1.00 | 0.00 | 3A4 |
| ATOM | 1340 | N | PHE | 218 | 10.374 | −12.933 | 18.064 | 1.00 | 0.00 | 3A4 |
| ATOM | 1341 | CA | PHE | 218 | 10.004 | −13.621 | 16.839 | 1.00 | 0.00 | 3A4 |
| ATOM | 1342 | CB | PHE | 218 | 8.877 | −14.691 | 17.032 | 1.00 | 0.00 | 3A4 |
| ATOM | 1343 | CG | PHE | 218 | 9.294 | −15.734 | 18.036 | 1.00 | 0.00 | 3A4 |
| ATOM | 1344 | CD1 | PHE | 218 | 8.630 | −15.831 | 19.273 | 1.00 | 0.00 | 3A4 |
| ATOM | 1345 | CD2 | PHE | 218 | 10.354 | −16.621 | 17.766 | 1.00 | 0.00 | 3A4 |
| ATOM | 1346 | CE1 | PHE | 218 | 9.018 | −16.783 | 20.225 | 1.00 | 0.00 | 3A4 |
| ATOM | 1347 | CE2 | PHE | 218 | 10.751 | −17.572 | 18.720 | 1.00 | 0.00 | 3A4 |
| ATOM | 1348 | CZ | PHE | 218 | 10.083 | −17.651 | 19.950 | 1.00 | 0.00 | 3A4 |
| ATOM | 1349 | C | PHE | 218 | 9.495 | −12.564 | 15.883 | 1.00 | 0.00 | 3A4 |
| ATOM | 1350 | O | PHE | 218 | 9.158 | −12.860 | 14.737 | 1.00 | 0.00 | 3A4 |
| ATOM | 1351 | N | PHE | 219 | 9.400 | −11.297 | 16.374 | 1.00 | 0.00 | 3A4 |
| ATOM | 1352 | CA | PHE | 219 | 8.762 | −10.165 | 15.736 | 1.00 | 0.00 | 3A4 |
| ATOM | 1353 | CB | PHE | 219 | 7.558 | −9.630 | 16.572 | 1.00 | 0.00 | 3A4 |
| ATOM | 1354 | CG | PHE | 219 | 6.443 | −10.646 | 16.570 | 1.00 | 0.00 | 3A4 |
| ATOM | 1355 | CD1 | PHE | 219 | 5.437 | −10.600 | 15.587 | 1.00 | 0.00 | 3A4 |
| ATOM | 1356 | CD2 | PHE | 219 | 6.389 | −11.664 | 17.542 | 1.00 | 0.00 | 3A4 |
| ATOM | 1357 | CE1 | PHE | 219 | 4.401 | −11.546 | 15.576 | 1.00 | 0.00 | 3A4 |
| ATOM | 1358 | CE2 | PHE | 219 | 5.356 | −12.611 | 17.536 | 1.00 | 0.00 | 3A4 |
| ATOM | 1359 | CZ | PHE | 219 | 4.360 | −12.551 | 16.553 | 1.00 | 0.00 | 3A4 |
| ATOM | 1360 | C | PHE | 219 | 9.798 | −9.078 | 15.602 | 1.00 | 0.00 | 3A4 |
| ATOM | 1361 | O | PHE | 219 | 10.805 | −9.068 | 16.307 | 1.00 | 0.00 | 3A4 |
| ATOM | 1362 | N | LEU | 220 | 9.547 | −8.131 | 14.664 | 1.00 | 0.00 | 3A4 |
| ATOM | 1363 | CA | LEU | 220 | 10.447 | −7.050 | 14.321 | 1.00 | 0.00 | 3A4 |
| ATOM | 1364 | CB | LEU | 220 | 11.550 | −7.452 | 13.279 | 1.00 | 0.00 | 3A4 |
| ATOM | 1365 | CG | LEU | 220 | 11.130 | −7.889 | 11.840 | 1.00 | 0.00 | 3A4 |
| ATOM | 1366 | CD1 | LEU | 220 | 12.368 | −7.938 | 10.924 | 1.00 | 0.00 | 3A4 |
| ATOM | 1367 | CD2 | LEU | 220 | 10.365 | −9.229 | 11.764 | 1.00 | 0.00 | 3A4 |
| ATOM | 1368 | C | LEU | 220 | 9.590 | −5.908 | 13.825 | 1.00 | 0.00 | 3A4 |
| ATOM | 1369 | O | LEU | 220 | 8.366 | −5.964 | 13.937 | 1.00 | 0.00 | 3A4 |
| ATOM | 1370 | N | SER | 221 | 10.268 | −4.849 | 13.284 | 1.00 | 0.00 | 3A4 |
| ATOM | 1371 | CA | SER | 221 | 9.781 | −3.584 | 12.735 | 1.00 | 0.00 | 3A4 |
| ATOM | 1372 | CB | SER | 221 | 8.270 | −3.542 | 12.288 | 1.00 | 0.00 | 3A4 |
| ATOM | 1373 | OG | SER | 221 | 7.980 | −2.485 | 11.376 | 1.00 | 0.00 | 3A4 |
| ATOM | 1374 | C | SER | 221 | 10.129 | −2.514 | 13.764 | 1.00 | 0.00 | 3A4 |
| ATOM | 1375 | O | SER | 221 | 10.958 | −2.740 | 14.646 | 1.00 | 0.00 | 3A4 |
| ATOM | 1376 | N | ILE | 222 | 9.495 | −1.312 | 13.661 | 1.00 | 0.00 | 3A4 |
| ATOM | 1377 | CA | ILE | 222 | 9.692 | −0.155 | 14.524 | 1.00 | 0.00 | 3A4 |
| ATOM | 1378 | CB | ILE | 222 | 9.886 | 1.145 | 13.735 | 1.00 | 0.00 | 3A4 |
| ATOM | 1379 | CG2 | ILE | 222 | 11.258 | 1.038 | 13.025 | 1.00 | 0.00 | 3A4 |
| ATOM | 1380 | CG1 | ILE | 222 | 8.727 | 1.449 | 12.741 | 1.00 | 0.00 | 3A4 |
| ATOM | 1381 | CD | ILE | 222 | 8.868 | 2.791 | 12.016 | 1.00 | 0.00 | 3A4 |
| ATOM | 1382 | C | ILE | 222 | 8.533 | −0.038 | 15.500 | 1.00 | 0.00 | 3A4 |
| ATOM | 1383 | O | ILE | 222 | 8.631 | 0.670 | 16.501 | 1.00 | 0.00 | 3A4 |
| ATOM | 1384 | N | THR | 223 | 7.406 | −0.746 | 15.215 | 1.00 | 0.00 | 3A4 |
| ATOM | 1385 | CA | THR | 223 | 6.225 | −0.787 | 16.050 | 1.00 | 0.00 | 3A4 |
| ATOM | 1386 | CB | THR | 223 | 5.259 | 0.384 | 15.782 | 1.00 | 0.00 | 3A4 |
| ATOM | 1387 | OG1 | THR | 223 | 4.159 | 0.414 | 16.691 | 1.00 | 0.00 | 3A4 |
| ATOM | 1388 | CG2 | THR | 223 | 4.751 | 0.459 | 14.318 | 1.00 | 0.00 | 3A4 |
| ATOM | 1389 | C | THR | 223 | 5.633 | −2.165 | 15.811 | 1.00 | 0.00 | 3A4 |
| ATOM | 1390 | O | THR | 223 | 5.360 | −2.558 | 14.678 | 1.00 | 0.00 | 3A4 |
| ATOM | 1391 | N | VAL | 224 | 5.435 | −2.927 | 16.918 | 1.00 | 0.00 | 3A4 |
| ATOM | 1392 | CA | VAL | 224 | 4.832 | −4.244 | 16.917 | 1.00 | 0.00 | 3A4 |
| ATOM | 1393 | CB | VAL | 224 | 5.751 | −5.354 | 16.379 | 1.00 | 0.00 | 3A4 |
| ATOM | 1394 | CG1 | VAL | 224 | 7.065 | −5.517 | 17.186 | 1.00 | 0.00 | 3A4 |
| ATOM | 1395 | CG2 | VAL | 224 | 4.973 | −6.679 | 16.198 | 1.00 | 0.00 | 3A4 |
| ATOM | 1396 | C | VAL | 224 | 4.397 | −4.468 | 18.349 | 1.00 | 0.00 | 3A4 |
| ATOM | 1397 | O | VAL | 224 | 5.085 | −4.071 | 19.290 | 1.00 | 0.00 | 3A4 |
| ATOM | 1398 | N | PHE | 225 | 3.212 | −5.114 | 18.534 | 1.00 | 0.00 | 3A4 |
| ATOM | 1399 | CA | PHE | 225 | 2.568 | −5.398 | 19.809 | 1.00 | 0.00 | 3A4 |
| ATOM | 1400 | CB | PHE | 225 | 1.025 | −5.112 | 19.736 | 1.00 | 0.00 | 3A4 |
| ATOM | 1401 | CG | PHE | 225 | 0.345 | −5.089 | 21.089 | 1.00 | 0.00 | 3A4 |
| ATOM | 1402 | CD1 | PHE | 225 | 0.584 | −4.036 | 21.993 | 1.00 | 0.00 | 3A4 |
| ATOM | 1403 | CD2 | PHE | 225 | −0.533 | −6.123 | 21.470 | 1.00 | 0.00 | 3A4 |
| ATOM | 1404 | CE1 | PHE | 225 | −0.038 | −4.017 | 23.251 | 1.00 | 0.00 | 3A4 |
| ATOM | 1405 | CE2 | PHE | 225 | −1.156 | −6.107 | 22.726 | 1.00 | 0.00 | 3A4 |
| ATOM | 1406 | CZ | PHE | 225 | −0.908 | −5.053 | 23.617 | 1.00 | 0.00 | 3A4 |
| ATOM | 1407 | C | PHE | 225 | 2.898 | −6.812 | 20.313 | 1.00 | 0.00 | 3A4 |
| ATOM | 1408 | O | PHE | 225 | 3.306 | −6.885 | 21.472 | 1.00 | 0.00 | 3A4 |
| ATOM | 1409 | N | PRO | 226 | 2.776 | −7.964 | 19.590 | 1.00 | 0.00 | 3A4 |
| ATOM | 1410 | CA | PRO | 226 | 3.087 | −9.298 | 20.117 | 1.00 | 0.00 | 3A4 |
| ATOM | 1411 | CD | PRO | 226 | 2.027 | −8.062 | 18.335 | 1.00 | 0.00 | 3A4 |
| ATOM | 1412 | CB | PRO | 226 | 2.324 | −10.268 | 19.186 | 1.00 | 0.00 | 3A4 |
| ATOM | 1413 | CG | PRO | 226 | 2.191 | −9.507 | 17.865 | 1.00 | 0.00 | 3A4 |
| ATOM | 1414 | C | PRO | 226 | 4.587 | −9.598 | 20.127 | 1.00 | 0.00 | 3A4 |
| ATOM | 1415 | O | PRO | 226 | 5.346 | −9.003 | 19.361 | 1.00 | 0.00 | 3A4 |
| ATOM | 1416 | N | PHE | 227 | 4.993 | −10.544 | 21.009 | 1.00 | 0.00 | 3A4 |
| ATOM | 1417 | CA | PHE | 227 | 6.356 | −10.995 | 21.212 | 1.00 | 0.00 | 3A4 |
| ATOM | 1418 | CB | PHE | 227 | 7.106 | −10.254 | 22.376 | 1.00 | 0.00 | 3A4 |
| ATOM | 1419 | CG | PHE | 227 | 6.298 | −10.129 | 23.651 | 1.00 | 0.00 | 3A4 |
| ATOM | 1420 | CD1 | PHE | 227 | 5.437 | −9.031 | 23.855 | 1.00 | 0.00 | 3A4 |
| ATOM | 1421 | CD2 | PHE | 227 | 6.393 | −11.105 | 24.663 | 1.00 | 0.00 | 3A4 |
| ATOM | 1422 | CE1 | PHE | 227 | 4.677 | −8.920 | 25.028 | 1.00 | 0.00 | 3A4 |
| ATOM | 1423 | CE2 | PHE | 227 | 5.634 | −10.999 | 25.838 | 1.00 | 0.00 | 3A4 |
| ATOM | 1424 | CZ | PHE | 227 | 4.775 | −9.907 | 26.019 | 1.00 | 0.00 | 3A4 |
| ATOM | 1425 | C | PHE | 227 | 6.307 | −12.495 | 21.429 | 1.00 | 0.00 | 3A4 |
| ATOM | 1426 | O | PHE | 227 | 7.291 | −13.188 | 21.177 | 1.00 | 0.00 | 3A4 |
| ATOM | 1427 | N | LEU | 228 | 5.134 | −13.001 | 21.925 | 1.00 | 0.00 | 3A4 |
| ATOM | 1428 | CA | LEU | 228 | 4.753 | −14.389 | 22.186 | 1.00 | 0.00 | 3A4 |
| ATOM | 1429 | CB | LEU | 228 | 5.137 | −15.381 | 21.024 | 1.00 | 0.00 | 3A4 |
| ATOM | 1430 | CG | LEU | 228 | 4.217 | −16.607 | 20.757 | 1.00 | 0.00 | 3A4 |
| ATOM | 1431 | CD1 | LEU | 228 | 4.267 | −17.707 | 21.832 | 1.00 | 0.00 | 3A4 |
| ATOM | 1432 | CD2 | LEU | 228 | 2.767 | −16.215 | 20.406 | 1.00 | 0.00 | 3A4 |
| ATOM | 1433 | C | LEU | 228 | 5.274 | −14.788 | 23.565 | 1.00 | 0.00 | 3A4 |
| ATOM | 1434 | O | LEU | 228 | 4.778 | −14.291 | 24.575 | 1.00 | 0.00 | 3A4 |
| ATOM | 1435 | N | ILE | 229 | 6.316 | −15.659 | 23.623 | 1.00 | 0.00 | 3A4 |
| ATOM | 1436 | CA | ILE | 229 | 7.097 | −15.948 | 24.811 | 1.00 | 0.00 | 3A4 |
| ATOM | 1437 | CB | ILE | 229 | 6.457 | −16.944 | 25.799 | 1.00 | 0.00 | 3A4 |
| ATOM | 1438 | CG2 | ILE | 229 | 6.056 | −18.311 | 25.188 | 1.00 | 0.00 | 3A4 |
| ATOM | 1439 | CG1 | ILE | 229 | 7.185 | −17.043 | 27.173 | 1.00 | 0.00 | 3A4 |
| ATOM | 1440 | CD | ILE | 229 | 8.372 | −18.014 | 27.278 | 1.00 | 0.00 | 3A4 |
| ATOM | 1441 | C | ILE | 229 | 8.458 | −16.351 | 24.252 | 1.00 | 0.00 | 3A4 |
| ATOM | 1442 | O | ILE | 229 | 8.631 | −17.503 | 23.857 | 1.00 | 0.00 | 3A4 |
| ATOM | 1443 | N | PRO | 230 | 9.474 | −15.455 | 24.157 | 1.00 | 0.00 | 3A4 |
| ATOM | 1444 | CA | PRO | 230 | 10.759 | −15.735 | 23.519 | 1.00 | 0.00 | 3A4 |
| ATOM | 1445 | CD | PRO | 230 | 9.306 | −14.022 | 24.410 | 1.00 | 0.00 | 3A4 |
| ATOM | 1446 | CB | PRO | 230 | 11.244 | −14.349 | 23.055 | 1.00 | 0.00 | 3A4 |
| ATOM | 1447 | CG | PRO | 230 | 10.655 | −13.376 | 24.082 | 1.00 | 0.00 | 3A4 |
| ATOM | 1448 | C | PRO | 230 | 11.749 | −16.380 | 24.483 | 1.00 | 0.00 | 3A4 |
| ATOM | 1449 | O | PRO | 230 | 11.509 | −16.422 | 25.691 | 1.00 | 0.00 | 3A4 |
| ATOM | 1450 | N | ILE | 231 | 12.894 | −16.870 | 23.931 | 1.00 | 0.00 | 3A4 |
| ATOM | 1451 | CA | ILE | 231 | 13.997 | −17.495 | 24.644 | 1.00 | 0.00 | 3A4 |
| ATOM | 1452 | CB | ILE | 231 | 14.463 | −18.802 | 23.988 | 1.00 | 0.00 | 3A4 |
| ATOM | 1453 | CG2 | ILE | 231 | 15.554 | −19.489 | 24.852 | 1.00 | 0.00 | 3A4 |
| ATOM | 1454 | CG1 | ILE | 231 | 13.275 | −19.767 | 23.714 | 1.00 | 0.00 | 3A4 |
| ATOM | 1455 | CD | ILE | 231 | 12.502 | −20.240 | 24.953 | 1.00 | 0.00 | 3A4 |
| ATOM | 1456 | C | ILE | 231 | 15.116 | −16.473 | 24.709 | 1.00 | 0.00 | 3A4 |
| ATOM | 1457 | O | ILE | 231 | 15.691 | −16.247 | 25.773 | 1.00 | 0.00 | 3A4 |
| ATOM | 1458 | N | LEU | 232 | 15.428 | −15.844 | 23.533 | 1.00 | 0.00 | 3A4 |
| ATOM | 1459 | CA | LEU | 232 | 16.397 | −14.778 | 23.277 | 1.00 | 0.00 | 3A4 |
| ATOM | 1460 | CB | LEU | 232 | 16.312 | −13.552 | 24.250 | 1.00 | 0.00 | 3A4 |
| ATOM | 1461 | CG | LEU | 232 | 14.944 | −12.824 | 24.269 | 1.00 | 0.00 | 3A4 |
| ATOM | 1462 | CD1 | LEU | 232 | 14.900 | −11.763 | 25.383 | 1.00 | 0.00 | 3A4 |
| ATOM | 1463 | CD2 | LEU | 232 | 14.567 | −12.203 | 22.908 | 1.00 | 0.00 | 3A4 |
| ATOM | 1464 | C | LEU | 232 | 17.814 | −15.328 | 23.232 | 1.00 | 0.00 | 3A4 |
| ATOM | 1465 | O | LEU | 232 | 18.606 | −15.133 | 24.154 | 1.00 | 0.00 | 3A4 |
| ATOM | 1466 | N | GLU | 233 | 18.131 | −16.047 | 22.125 | 1.00 | 0.00 | 3A4 |
| ATOM | 1467 | CA | GLU | 233 | 19.390 | −16.728 | 21.896 | 1.00 | 0.00 | 3A4 |
| ATOM | 1468 | CB | GLU | 233 | 19.269 | −18.268 | 22.115 | 1.00 | 0.00 | 3A4 |
| ATOM | 1469 | CG | GLU | 233 | 18.095 | −18.955 | 21.373 | 1.00 | 0.00 | 3A4 |
| ATOM | 1470 | CD | GLU | 233 | 18.120 | −20.462 | 21.648 | 1.00 | 0.00 | 3A4 |
| ATOM | 1471 | OE1 | GLU | 233 | 19.140 | −21.114 | 21.295 | 1.00 | 0.00 | 3A4 |
| ATOM | 1472 | OE2 | GLU | 233 | 17.118 | −20.985 | 22.205 | 1.00 | 0.00 | 3A4 |
| ATOM | 1473 | C | GLU | 233 | 19.828 | −16.395 | 20.489 | 1.00 | 0.00 | 3A4 |
| ATOM | 1474 | O | GLU | 233 | 20.256 | −17.261 | 19.726 | 1.00 | 0.00 | 3A4 |
| ATOM | 1475 | N | VAL | 234 | 19.734 | −15.088 | 20.125 | 1.00 | 0.00 | 3A4 |
| ATOM | 1476 | CA | VAL | 234 | 20.053 | −14.558 | 18.815 | 1.00 | 0.00 | 3A4 |
| ATOM | 1477 | CB | VAL | 234 | 18.792 | −14.275 | 17.987 | 1.00 | 0.00 | 3A4 |
| ATOM | 1478 | CG1 | VAL | 234 | 17.739 | −13.402 | 18.723 | 1.00 | 0.00 | 3A4 |
| ATOM | 1479 | CG2 | VAL | 234 | 19.142 | −13.768 | 16.568 | 1.00 | 0.00 | 3A4 |
| ATOM | 1480 | C | VAL | 234 | 20.960 | −13.370 | 19.064 | 1.00 | 0.00 | 3A4 |
| ATOM | 1481 | O | VAL | 234 | 20.563 | −12.209 | 18.984 | 1.00 | 0.00 | 3A4 |
| ATOM | 1482 | N | LEU | 235 | 22.242 | −13.673 | 19.393 | 1.00 | 0.00 | 3A4 |
| ATOM | 1483 | CA | LEU | 235 | 23.270 | −12.694 | 19.675 | 1.00 | 0.00 | 3A4 |
| ATOM | 1484 | CB | LEU | 235 | 23.137 | −11.954 | 21.056 | 1.00 | 0.00 | 3A4 |
| ATOM | 1485 | CG | LEU | 235 | 23.294 | −12.749 | 22.393 | 1.00 | 0.00 | 3A4 |
| ATOM | 1486 | CD1 | LEU | 235 | 23.236 | −11.785 | 23.594 | 1.00 | 0.00 | 3A4 |
| ATOM | 1487 | CD2 | LEU | 235 | 22.289 | −13.903 | 22.600 | 1.00 | 0.00 | 3A4 |
| ATOM | 1488 | C | LEU | 235 | 24.592 | −13.413 | 19.593 | 1.00 | 0.00 | 3A4 |
| ATOM | 1489 | O | LEU | 235 | 24.703 | −14.586 | 19.948 | 1.00 | 0.00 | 3A4 |
| ATOM | 1490 | N | ASN | 236 | 25.638 | −12.684 | 19.128 | 1.00 | 0.00 | 3A4 |
| ATOM | 1491 | CA | ASN | 236 | 27.013 | −13.135 | 19.089 | 1.00 | 0.00 | 3A4 |
| ATOM | 1492 | CB | ASN | 236 | 27.351 | −13.966 | 17.803 | 1.00 | 0.00 | 3A4 |
| ATOM | 1493 | CG | ASN | 236 | 28.697 | −14.706 | 17.931 | 1.00 | 0.00 | 3A4 |
| ATOM | 1494 | OD1 | ASN | 236 | 28.818 | −15.613 | 18.753 | 1.00 | 0.00 | 3A4 |
| ATOM | 1495 | ND2 | ASN | 236 | 29.719 | −14.326 | 17.115 | 1.00 | 0.00 | 3A4 |
| ATOM | 1496 | C | ASN | 236 | 27.795 | −11.845 | 19.165 | 1.00 | 0.00 | 3A4 |
| ATOM | 1497 | O | ASN | 236 | 28.546 | −11.489 | 18.259 | 1.00 | 0.00 | 3A4 |
| ATOM | 1498 | N | ILE | 237 | 27.590 | −11.096 | 20.283 | 1.00 | 0.00 | 3A4 |
| ATOM | 1499 | CA | ILE | 237 | 28.074 | −9.747 | 20.504 | 1.00 | 0.00 | 3A4 |
| ATOM | 1500 | CB | ILE | 237 | 26.980 | −8.800 | 20.998 | 1.00 | 0.00 | 3A4 |
| ATOM | 1501 | CG2 | ILE | 237 | 26.039 | −8.584 | 19.789 | 1.00 | 0.00 | 3A4 |
| ATOM | 1502 | CG1 | ILE | 237 | 26.203 | −9.271 | 22.262 | 1.00 | 0.00 | 3A4 |
| ATOM | 1503 | CD | ILE | 237 | 25.059 | −8.341 | 22.671 | 1.00 | 0.00 | 3A4 |
| ATOM | 1504 | C | ILE | 237 | 29.228 | −9.798 | 21.461 | 1.00 | 0.00 | 3A4 |
| ATOM | 1505 | O | ILE | 237 | 30.202 | −9.126 | 21.226 | 1.00 | 0.00 | 3A4 |
| ATOM | 1506 | N | CYS | 238 | 29.124 | −10.614 | 22.532 | 1.00 | 0.00 | 3A4 |
| ATOM | 1507 | CA | CYS | 238 | 30.099 | −10.834 | 23.576 | 1.00 | 0.00 | 3A4 |
| ATOM | 1508 | CB | CYS | 238 | 29.513 | −11.689 | 24.739 | 1.00 | 0.00 | 3A4 |
| ATOM | 1509 | SG | CYS | 238 | 28.106 | −10.870 | 25.526 | 1.00 | 0.00 | 3A4 |
| ATOM | 1510 | C | CYS | 238 | 31.307 | −11.563 | 23.083 | 1.00 | 0.00 | 3A4 |
| ATOM | 1511 | O | CYS | 238 | 32.424 | −11.278 | 23.493 | 1.00 | 0.00 | 3A4 |
| ATOM | 1512 | N | VAL | 239 | 31.138 | −12.446 | 22.082 | 1.00 | 0.00 | 3A4 |
| ATOM | 1513 | CA | VAL | 239 | 32.219 | −13.161 | 21.438 | 1.00 | 0.00 | 3A4 |
| ATOM | 1514 | CB | VAL | 239 | 31.700 | −14.367 | 20.675 | 1.00 | 0.00 | 3A4 |
| ATOM | 1515 | CG1 | VAL | 239 | 32.858 | −15.308 | 20.247 | 1.00 | 0.00 | 3A4 |
| ATOM | 1516 | CG2 | VAL | 239 | 30.715 | −15.139 | 21.586 | 1.00 | 0.00 | 3A4 |
| ATOM | 1517 | C | VAL | 239 | 33.006 | −12.283 | 20.505 | 1.00 | 0.00 | 3A4 |
| ATOM | 1518 | O | VAL | 239 | 34.228 | −12.327 | 20.485 | 1.00 | 0.00 | 3A4 |
| ATOM | 1519 | N | PHE | 240 | 32.315 | −11.420 | 19.731 | 1.00 | 0.00 | 3A4 |
| ATOM | 1520 | CA | PHE | 240 | 32.913 | −10.422 | 18.870 | 1.00 | 0.00 | 3A4 |
| ATOM | 1521 | CB | PHE | 240 | 31.825 | −9.669 | 18.040 | 1.00 | 0.00 | 3A4 |
| ATOM | 1522 | CG | PHE | 240 | 31.881 | −8.134 | 17.899 | 1.00 | 0.00 | 3A4 |
| ATOM | 1523 | CD1 | PHE | 240 | 32.913 | −7.550 | 17.149 | 1.00 | 0.00 | 3A4 |
| ATOM | 1524 | CD2 | PHE | 240 | 31.185 | −7.320 | 18.812 | 1.00 | 0.00 | 3A4 |
| ATOM | 1525 | CE1 | PHE | 240 | 33.312 | −6.223 | 17.382 | 1.00 | 0.00 | 3A4 |
| ATOM | 1526 | CE2 | PHE | 240 | 31.646 | −6.034 | 19.117 | 1.00 | 0.00 | 3A4 |
| ATOM | 1527 | CZ | PHE | 240 | 32.709 | −5.481 | 18.396 | 1.00 | 0.00 | 3A4 |
| ATOM | 1528 | C | PHE | 240 | 33.789 | −9.440 | 19.608 | 1.00 | 0.00 | 3A4 |
| ATOM | 1529 | O | PHE | 240 | 34.906 | −9.202 | 19.168 | 1.00 | 0.00 | 3A4 |
| ATOM | 1530 | N | PRO | 241 | 33.347 | −8.835 | 20.727 | 1.00 | 0.00 | 3A4 |
| ATOM | 1531 | CA | PRO | 241 | 34.111 | −7.940 | 21.493 | 1.00 | 0.00 | 3A4 |
| ATOM | 1532 | CD | PRO | 241 | 32.648 | −9.345 | 21.724 | 1.00 | 0.00 | 3A4 |
| ATOM | 1533 | CB | PRO | 241 | 33.196 | −7.358 | 22.578 | 1.00 | 0.00 | 3A4 |
| ATOM | 1534 | CG | PRO | 241 | 32.348 | −8.435 | 22.936 | 1.00 | 0.00 | 3A4 |
| ATOM | 1535 | C | PRO | 241 | 35.347 | −8.605 | 22.103 | 1.00 | 0.00 | 3A4 |
| ATOM | 1536 | O | PRO | 241 | 36.396 | −7.992 | 22.029 | 1.00 | 0.00 | 3A4 |
| ATOM | 1537 | N | ARG | 242 | 35.316 | −9.867 | 22.592 | 1.00 | 0.00 | 3A4 |
| ATOM | 1538 | CA | ARG | 242 | 36.450 | −10.583 | 23.101 | 1.00 | 0.00 | 3A4 |
| ATOM | 1539 | CB | ARG | 242 | 35.986 | −11.889 | 23.750 | 1.00 | 0.00 | 3A4 |
| ATOM | 1540 | CG | ARG | 242 | 35.213 | −11.657 | 25.054 | 1.00 | 0.00 | 3A4 |
| ATOM | 1541 | CD | ARG | 242 | 34.515 | −12.928 | 25.556 | 1.00 | 0.00 | 3A4 |
| ATOM | 1542 | NE | ARG | 242 | 33.717 | −12.581 | 26.784 | 1.00 | 0.00 | 3A4 |
| ATOM | 1543 | CZ | ARG | 242 | 32.762 | −13.412 | 27.314 | 1.00 | 0.00 | 3A4 |
| ATOM | 1544 | NH1 | ARG | 242 | 32.114 | −13.042 | 28.455 | 1.00 | 0.00 | 3A4 |
| ATOM | 1545 | NH2 | ARG | 242 | 32.444 | −14.603 | 26.725 | 1.00 | 0.00 | 3A4 |
| ATOM | 1546 | C | ARG | 242 | 37.483 | −10.871 | 22.037 | 1.00 | 0.00 | 3A4 |
| ATOM | 1547 | O | ARG | 242 | 38.677 | −10.761 | 22.298 | 1.00 | 0.00 | 3A4 |
| ATOM | 1548 | N | GLU | 243 | 37.044 | −11.158 | 20.784 | 1.00 | 0.00 | 3A4 |
| ATOM | 1549 | CA | GLU | 243 | 37.911 | −11.394 | 19.638 | 1.00 | 0.00 | 3A4 |
| ATOM | 1550 | CB | GLU | 243 | 37.126 | −11.846 | 18.383 | 1.00 | 0.00 | 3A4 |
| ATOM | 1551 | CG | GLU | 243 | 36.626 | −13.298 | 18.452 | 1.00 | 0.00 | 3A4 |
| ATOM | 1552 | CD | GLU | 243 | 35.759 | −13.581 | 17.219 | 1.00 | 0.00 | 3A4 |
| ATOM | 1553 | OE1 | GLU | 243 | 34.528 | −13.792 | 17.385 | 1.00 | 0.00 | 3A4 |
| ATOM | 1554 | OE2 | GLU | 243 | 36.319 | −13.581 | 16.089 | 1.00 | 0.00 | 3A4 |
| ATOM | 1555 | C | GLU | 243 | 38.703 | −10.164 | 19.275 | 1.00 | 0.00 | 3A4 |
| ATOM | 1556 | O | GLU | 243 | 39.908 | −10.244 | 19.057 | 1.00 | 0.00 | 3A4 |
| ATOM | 1557 | N | VAL | 244 | 38.043 | −8.981 | 19.287 | 1.00 | 0.00 | 3A4 |
| ATOM | 1558 | CA | VAL | 244 | 38.684 | −7.714 | 18.990 | 1.00 | 0.00 | 3A4 |
| ATOM | 1559 | CB | VAL | 244 | 37.676 | −6.607 | 18.731 | 1.00 | 0.00 | 3A4 |
| ATOM | 1560 | CG1 | VAL | 244 | 38.391 | −5.314 | 18.259 | 1.00 | 0.00 | 3A4 |
| ATOM | 1561 | CG2 | VAL | 244 | 36.813 | −7.052 | 17.545 | 1.00 | 0.00 | 3A4 |
| ATOM | 1562 | C | VAL | 244 | 39.649 | −7.327 | 20.084 | 1.00 | 0.00 | 3A4 |
| ATOM | 1563 | O | VAL | 244 | 40.771 | −6.912 | 19.811 | 1.00 | 0.00 | 3A4 |
| ATOM | 1564 | N | THR | 245 | 39.257 | −7.535 | 21.357 | 1.00 | 0.00 | 3A4 |
| ATOM | 1565 | CA | THR | 245 | 40.070 | −7.264 | 22.527 | 1.00 | 0.00 | 3A4 |
| ATOM | 1566 | CB | THR | 245 | 39.245 | −7.434 | 23.795 | 1.00 | 0.00 | 3A4 |
| ATOM | 1567 | OG1 | THR | 245 | 38.211 | −6.455 | 23.817 | 1.00 | 0.00 | 3A4 |
| ATOM | 1568 | CG2 | THR | 245 | 40.041 | −7.330 | 25.127 | 1.00 | 0.00 | 3A4 |
| ATOM | 1569 | C | THR | 245 | 41.317 | −8.113 | 22.572 | 1.00 | 0.00 | 3A4 |
| ATOM | 1570 | O | THR | 245 | 42.397 | −7.605 | 22.842 | 1.00 | 0.00 | 3A4 |
| ATOM | 1571 | N | ASN | 246 | 41.225 | −9.408 | 22.207 | 1.00 | 0.00 | 3A4 |
| ATOM | 1572 | CA | ASN | 246 | 42.344 | −10.327 | 22.152 | 1.00 | 0.00 | 3A4 |
| ATOM | 1573 | CB | ASN | 246 | 41.877 | −11.774 | 21.874 | 1.00 | 0.00 | 3A4 |
| ATOM | 1574 | CG | ASN | 246 | 41.116 | −12.349 | 23.082 | 1.00 | 0.00 | 3A4 |
| ATOM | 1575 | OD1 | ASN | 246 | 41.128 | −11.808 | 24.187 | 1.00 | 0.00 | 3A4 |
| ATOM | 1576 | ND2 | ASN | 246 | 40.427 | −13.502 | 22.862 | 1.00 | 0.00 | 3A4 |
| ATOM | 1577 | C | ASN | 246 | 43.340 | −9.950 | 21.088 | 1.00 | 0.00 | 3A4 |
| ATOM | 1578 | O | ASN | 246 | 44.544 | −10.003 | 21.305 | 1.00 | 0.00 | 3A4 |
| ATOM | 1579 | N | PHE | 247 | 42.846 | −9.491 | 19.921 | 1.00 | 0.00 | 3A4 |
| ATOM | 1580 | CA | PHE | 247 | 43.640 | −9.006 | 18.820 | 1.00 | 0.00 | 3A4 |
| ATOM | 1581 | CB | PHE | 247 | 42.712 | −8.691 | 17.625 | 1.00 | 0.00 | 3A4 |
| ATOM | 1582 | CG | PHE | 247 | 43.520 | −8.335 | 16.378 | 1.00 | 0.00 | 3A4 |
| ATOM | 1583 | CD1 | PHE | 247 | 44.084 | −9.348 | 15.580 | 1.00 | 0.00 | 3A4 |
| ATOM | 1584 | CD2 | PHE | 247 | 43.817 | −6.990 | 16.065 | 1.00 | 0.00 | 3A4 |
| ATOM | 1585 | CE1 | PHE | 247 | 44.943 | −9.028 | 14.517 | 1.00 | 0.00 | 3A4 |
| ATOM | 1586 | CE2 | PHE | 247 | 44.673 | −6.665 | 15.008 | 1.00 | 0.00 | 3A4 |
| ATOM | 1587 | CZ | PHE | 247 | 45.245 | −7.686 | 14.243 | 1.00 | 0.00 | 3A4 |
| ATOM | 1588 | C | PHE | 247 | 44.438 | −7.768 | 19.171 | 1.00 | 0.00 | 3A4 |
| ATOM | 1589 | O | PHE | 247 | 45.631 | −7.693 | 18.897 | 1.00 | 0.00 | 3A4 |
| ATOM | 1590 | N | LEU | 248 | 43.781 | −6.766 | 19.795 | 1.00 | 0.00 | 3A4 |
| ATOM | 1591 | CA | LEU | 248 | 44.374 | −5.480 | 20.131 | 1.00 | 0.00 | 3A4 |
| ATOM | 1592 | CB | LEU | 248 | 43.291 | −4.473 | 20.599 | 1.00 | 0.00 | 3A4 |
| ATOM | 1593 | CG | LEU | 248 | 42.423 | −3.985 | 19.422 | 1.00 | 0.00 | 3A4 |
| ATOM | 1594 | CD1 | LEU | 248 | 41.143 | −3.318 | 19.920 | 1.00 | 0.00 | 3A4 |
| ATOM | 1595 | CD2 | LEU | 248 | 43.179 | −3.055 | 18.448 | 1.00 | 0.00 | 3A4 |
| ATOM | 1596 | C | LEU | 248 | 45.407 | −5.587 | 21.219 | 1.00 | 0.00 | 3A4 |
| ATOM | 1597 | O | LEU | 248 | 46.458 | −4.959 | 21.175 | 1.00 | 0.00 | 3A4 |
| ATOM | 1598 | N | ARG | 249 | 45.152 | −6.483 | 22.189 | 1.00 | 0.00 | 3A4 |
| ATOM | 1599 | CA | ARG | 249 | 46.055 | −6.805 | 23.263 | 1.00 | 0.00 | 3A4 |
| ATOM | 1600 | CB | ARG | 249 | 45.402 | −7.886 | 24.160 | 1.00 | 0.00 | 3A4 |
| ATOM | 1601 | CG | ARG | 249 | 44.526 | −7.297 | 25.274 | 1.00 | 0.00 | 3A4 |
| ATOM | 1602 | CD | ARG | 249 | 45.372 | −6.731 | 26.426 | 1.00 | 0.00 | 3A4 |
| ATOM | 1603 | NE | ARG | 249 | 44.462 | −6.210 | 27.502 | 1.00 | 0.00 | 3A4 |
| ATOM | 1604 | CZ | ARG | 249 | 44.932 | −5.795 | 28.722 | 1.00 | 0.00 | 3A4 |
| ATOM | 1605 | NH1 | ARG | 249 | 44.068 | −5.248 | 29.621 | 1.00 | 0.00 | 3A4 |
| ATOM | 1606 | NH2 | ARG | 249 | 46.251 | −5.917 | 29.056 | 1.00 | 0.00 | 3A4 |
| ATOM | 1607 | C | ARG | 249 | 47.418 | −7.301 | 22.846 | 1.00 | 0.00 | 3A4 |
| ATOM | 1608 | O | ARG | 249 | 48.444 | −6.897 | 23.385 | 1.00 | 0.00 | 3A4 |
| ATOM | 1609 | N | LYS | 250 | 47.456 | −8.139 | 21.790 | 1.00 | 0.00 | 3A4 |
| ATOM | 1610 | CA | LYS | 250 | 48.664 | −8.643 | 21.183 | 1.00 | 0.00 | 3A4 |
| ATOM | 1611 | CB | LYS | 250 | 48.353 | −9.760 | 20.149 | 1.00 | 0.00 | 3A4 |
| ATOM | 1612 | CG | LYS | 250 | 47.735 | −11.007 | 20.790 | 1.00 | 0.00 | 3A4 |
| ATOM | 1613 | CD | LYS | 250 | 47.184 | −11.995 | 19.759 | 1.00 | 0.00 | 3A4 |
| ATOM | 1614 | CE | LYS | 250 | 46.472 | −13.190 | 20.404 | 1.00 | 0.00 | 3A4 |
| ATOM | 1615 | NZ | LYS | 250 | 45.925 | −14.102 | 19.372 | 1.00 | 0.00 | 3A4 |
| ATOM | 1616 | C | LYS | 250 | 49.481 | −7.572 | 20.506 | 1.00 | 0.00 | 3A4 |
| ATOM | 1617 | O | LYS | 250 | 50.699 | −7.572 | 20.583 | 1.00 | 0.00 | 3A4 |
| ATOM | 1618 | N | SER | 251 | 48.809 | −6.584 | 19.881 | 1.00 | 0.00 | 3A4 |
| ATOM | 1619 | CA | SER | 251 | 49.413 | −5.423 | 19.268 | 1.00 | 0.00 | 3A4 |
| ATOM | 1620 | CB | SER | 251 | 48.350 | −4.598 | 18.498 | 1.00 | 0.00 | 3A4 |
| ATOM | 1621 | OG | SER | 251 | 47.705 | −5.410 | 17.524 | 1.00 | 0.00 | 3A4 |
| ATOM | 1622 | C | SER | 251 | 50.056 | −4.492 | 20.271 | 1.00 | 0.00 | 3A4 |
| ATOM | 1623 | O | SER | 251 | 51.163 | −4.000 | 20.091 | 1.00 | 0.00 | 3A4 |
| ATOM | 1624 | N | VAL | 252 | 49.376 | −4.275 | 21.416 | 1.00 | 0.00 | 3A4 |
| ATOM | 1625 | CA | VAL | 252 | 49.809 | −3.398 | 22.495 | 1.00 | 0.00 | 3A4 |
| ATOM | 1626 | CB | VAL | 252 | 40.673 | −3.198 | 23.486 | 1.00 | 0.00 | 3A4 |
| ATOM | 1627 | CG1 | VAL | 252 | 49.132 | −2.497 | 24.775 | 1.00 | 0.00 | 3A4 |
| ATOM | 1628 | CG2 | VAL | 252 | 47.610 | −2.462 | 22.625 | 1.00 | 0.00 | 3A4 |
| ATOM | 1629 | C | VAL | 252 | 51.052 | −3.938 | 23.163 | 1.00 | 0.00 | 3A4 |
| ATOM | 1630 | O | VAL | 252 | 51.998 | −3.206 | 23.419 | 1.00 | 0.00 | 3A4 |
| ATOM | 1631 | N | LYS | 253 | 51.108 | −5.271 | 23.380 | 1.00 | 0.00 | 3A4 |
| ATOM | 1632 | CA | LYS | 253 | 52.243 | −5.976 | 23.944 | 1.00 | 0.00 | 3A4 |
| ATOM | 1633 | CB | LYS | 253 | 51.887 | −7.462 | 24.155 | 1.00 | 0.00 | 3A4 |
| ATOM | 1634 | CG | LYS | 253 | 52.903 | −8.297 | 24.953 | 1.00 | 0.00 | 3A4 |
| ATOM | 1635 | CD | LYS | 253 | 52.410 | −9.721 | 25.224 | 1.00 | 0.00 | 3A4 |
| ATOM | 1636 | CE | LYS | 253 | 53.418 | −10.563 | 26.018 | 1.00 | 0.00 | 3A4 |
| ATOM | 1637 | NZ | LYS | 253 | 52.893 | −11.929 | 26.256 | 1.00 | 0.00 | 3A4 |
| ATOM | 1638 | C | LYS | 253 | 53.481 | −5.877 | 23.082 | 1.00 | 0.00 | 3A4 |
| ATOM | 1639 | O | LYS | 253 | 54.570 | −5.578 | 23.557 | 1.00 | 0.00 | 3A4 |
| ATOM | 1640 | N | ARG | 254 | 53.316 | −6.057 | 21.752 | 1.00 | 0.00 | 3A4 |
| ATOM | 1641 | CA | ARG | 254 | 54.372 | −5.928 | 20.768 | 1.00 | 0.00 | 3A4 |
| ATOM | 1642 | CB | ARG | 254 | 53.887 | −6.368 | 19.369 | 1.00 | 0.00 | 3A4 |
| ATOM | 1643 | CG | ARG | 254 | 53.631 | −7.877 | 19.278 | 1.00 | 0.00 | 3A4 |
| ATOM | 1644 | CD | ARG | 254 | 52.822 | −8.252 | 18.031 | 1.00 | 0.00 | 3A4 |
| ATOM | 1645 | NE | ARG | 254 | 52.473 | −9.714 | 18.098 | 1.00 | 0.00 | 3A4 |
| ATOM | 1646 | CZ | ARG | 254 | 51.381 | −10.256 | 17.466 | 1.00 | 0.00 | 3A4 |
| ATOM | 1647 | NH1 | ARG | 254 | 51.133 | −11.591 | 17.596 | 1.00 | 0.00 | 3A4 |
| ATOM | 1648 | NH2 | ARG | 254 | 50.529 | −9.490 | 16.721 | 1.00 | 0.00 | 3A4 |
| ATOM | 1649 | C | ARG | 254 | 54.899 | −4.517 | 20.676 | 1.00 | 0.00 | 3A4 |
| ATOM | 1650 | O | ARG | 254 | 56.093 | −4.310 | 20.498 | 1.00 | 0.00 | 3A4 |
| ATOM | 1651 | N | MET | 255 | 54.021 | −3.508 | 20.844 | 1.00 | 0.00 | 3A4 |
| ATOM | 1652 | CA | MET | 255 | 54.372 | −2.106 | 20.777 | 1.00 | 0.00 | 3A4 |
| ATOM | 1653 | CB | MET | 255 | 53.141 | −1.258 | 20.406 | 1.00 | 0.00 | 3A4 |
| ATOM | 1654 | CG | MET | 255 | 52.816 | −1.403 | 18.917 | 1.00 | 0.00 | 3A4 |
| ATOM | 1655 | SD | MET | 255 | 51.250 | −0.639 | 18.447 | 1.00 | 0.00 | 3A4 |
| ATOM | 1656 | CE | MET | 255 | 51.470 | −0.681 | 16.649 | 1.00 | 0.00 | 3A4 |
| ATOM | 1657 | C | MET | 255 | 55.008 | −1.583 | 22.041 | 1.00 | 0.00 | 3A4 |
| ATOM | 1658 | O | MET | 255 | 55.687 | −0.563 | 22.027 | 1.00 | 0.00 | 3A4 |
| ATOM | 1659 | N | LYS | 256 | 54.853 | −2.296 | 23.174 | 1.00 | 0.00 | 3A4 |
| ATOM | 1660 | CA | LYS | 256 | 55.524 | −1.998 | 24.424 | 1.00 | 0.00 | 3A4 |
| ATOM | 1661 | CB | LYS | 256 | 54.809 | −2.661 | 25.623 | 1.00 | 0.00 | 3A4 |
| ATOM | 1662 | CG | LYS | 256 | 53.537 | −1.942 | 26.076 | 1.00 | 0.00 | 3A4 |
| ATOM | 1663 | CD | LYS | 256 | 52.726 | −2.649 | 27.168 | 1.00 | 0.00 | 3A4 |
| ATOM | 1664 | CE | LYS | 256 | 53.383 | −2.616 | 28.556 | 1.00 | 0.00 | 3A4 |
| ATOM | 1665 | NZ | LYS | 256 | 52.487 | −3.206 | 29.579 | 1.00 | 0.00 | 3A4 |
| ATOM | 1666 | C | LYS | 256 | 56.960 | −2.488 | 24.397 | 1.00 | 0.00 | 3A4 |
| ATOM | 1667 | O | LYS | 256 | 57.837 | −1.909 | 25.029 | 1.00 | 0.00 | 3A4 |
| ATOM | 1668 | N | GLU | 257 | 57.220 | −3.565 | 23.619 | 1.00 | 0.00 | 3A4 |
| ATOM | 1669 | CA | GLU | 257 | 58.527 | −4.164 | 23.444 | 1.00 | 0.00 | 3A4 |
| ATOM | 1670 | CB | GLU | 257 | 58.421 | −5.661 | 23.058 | 1.00 | 0.00 | 3A4 |
| ATOM | 1671 | CG | GLU | 257 | 57.813 | −6.520 | 24.182 | 1.00 | 0.00 | 3A4 |
| ATOM | 1672 | CD | GLU | 257 | 57.607 | −7.953 | 23.679 | 1.00 | 0.00 | 3A4 |
| ATOM | 1673 | OE1 | GLU | 257 | 56.428 | −8.395 | 23.604 | 1.00 | 0.00 | 3A4 |
| ATOM | 1674 | OE2 | GLU | 257 | 58.625 | −8.625 | 23.363 | 1.00 | 0.00 | 3A4 |
| ATOM | 1675 | C | GLU | 257 | 59.381 | −3.451 | 22.410 | 1.00 | 0.00 | 3A4 |
| ATOM | 1676 | O | GLU | 257 | 60.407 | −2.870 | 22.760 | 1.00 | 0.00 | 3A4 |
| ATOM | 1677 | N | SER | 258 | 59.003 | −3.527 | 21.108 | 1.00 | 0.00 | 3A4 |
| ATOM | 1678 | CA | SER | 258 | 59.766 | −2.938 | 20.026 | 1.00 | 0.00 | 3A4 |
| ATOM | 1679 | CB | SER | 258 | 61.186 | −3.589 | 19.807 | 1.00 | 0.00 | 3A4 |
| ATOM | 1680 | OG | SER | 258 | 62.002 | −2.850 | 18.899 | 1.00 | 0.00 | 3A4 |
| ATOM | 1681 | C | SER | 258 | 58.938 | −3.119 | 18.776 | 1.00 | 0.00 | 3A4 |
| ATOM | 1682 | O | SER | 258 | 58.503 | −4.225 | 18.459 | 1.00 | 0.00 | 3A4 |
| ATOM | 1683 | N | ARG | 259 | 58.779 | −2.011 | 18.007 | 1.00 | 0.00 | 3A4 |
| ATOM | 1684 | CA | ARG | 259 | 58.391 | −2.013 | 16.612 | 1.00 | 0.00 | 3A4 |
| ATOM | 1685 | CB | ARG | 259 | 56.880 | −1.758 | 16.329 | 1.00 | 0.00 | 3A4 |
| ATOM | 1686 | CG | ARG | 259 | 55.968 | −2.939 | 16.688 | 1.00 | 0.00 | 3A4 |
| ATOM | 1687 | CD | ARG | 259 | 54.607 | −2.858 | 15.987 | 1.00 | 0.00 | 3A4 |
| ATOM | 1688 | NE | ARG | 259 | 53.744 | −4.000 | 16.458 | 1.00 | 0.00 | 3A4 |
| ATOM | 1689 | CZ | ARG | 259 | 52.617 | −4.411 | 15.791 | 1.00 | 0.00 | 3A4 |
| ATOM | 1690 | NH1 | ARG | 259 | 51.837 | −5.390 | 16.332 | 1.00 | 0.00 | 3A4 |
| ATOM | 1691 | NH2 | ARG | 259 | 52.247 | −3.859 | 14.598 | 1.00 | 0.00 | 3A4 |
| ATOM | 1692 | C | ARG | 259 | 59.262 | −0.943 | 16.000 | 1.00 | 0.00 | 3A4 |
| ATOM | 1693 | O | ARG | 259 | 60.401 | −1.220 | 15.626 | 1.00 | 0.00 | 3A4 |
| ATOM | 1694 | N | LEU | 260 | 58.752 | 0.314 | 15.898 | 1.00 | 0.00 | 3A4 |
| ATOM | 1695 | CA | LEU | 260 | 59.502 | 1.436 | 15.373 | 1.00 | 0.00 | 3A4 |
| ATOM | 1696 | CB | LEU | 260 | 59.602 | 1.425 | 13.811 | 1.00 | 0.00 | 3A4 |
| ATOM | 1697 | CG | LEU | 260 | 60.595 | 2.428 | 13.175 | 1.00 | 0.00 | 3A4 |
| ATOM | 1698 | CD1 | LEU | 260 | 62.054 | 2.163 | 13.599 | 1.00 | 0.00 | 3A4 |
| ATOM | 1699 | CD2 | LEU | 260 | 60.467 | 2.427 | 11.640 | 1.00 | 0.00 | 3A4 |
| ATOM | 1700 | C | LEU | 260 | 58.790 | 2.670 | 15.874 | 1.00 | 0.00 | 3A4 |
| ATOM | 1701 | O | LEU | 260 | 58.005 | 3.291 | 15.159 | 1.00 | 0.00 | 3A4 |
| ATOM | 1702 | N | GLU | 261 | 59.062 | 3.038 | 17.155 | 1.00 | 0.00 | 3A4 |
| ATOM | 1703 | CA | GLU | 261 | 58.441 | 4.130 | 17.884 | 1.00 | 0.00 | 3A4 |
| ATOM | 1704 | CB | GLU | 261 | 57.742 | 3.683 | 19.213 | 1.00 | 0.00 | 3A4 |
| ATOM | 1705 | CG | GLU | 261 | 58.554 | 2.841 | 20.229 | 1.00 | 0.00 | 3A4 |
| ATOM | 1706 | CD | GLU | 261 | 58.717 | 1.386 | 19.761 | 1.00 | 0.00 | 3A4 |
| ATOM | 1707 | OE1 | GLU | 261 | 59.884 | 0.942 | 19.589 | 1.00 | 0.00 | 3A4 |
| ATOM | 1708 | OE2 | GLU | 261 | 57.675 | 0.701 | 19.572 | 1.00 | 0.00 | 3A4 |
| ATOM | 1709 | C | GLU | 261 | 59.482 | 5.186 | 18.166 | 1.00 | 0.00 | 3A4 |
| ATOM | 1710 | O | GLU | 261 | 60.619 | 4.877 | 18.523 | 1.00 | 0.00 | 3A4 |
| ATOM | 1711 | N | ASP | 262 | 59.067 | 6.478 | 17.991 | 1.00 | 0.00 | 3A4 |
| ATOM | 1712 | CA | ASP | 262 | 59.771 | 7.738 | 18.239 | 1.00 | 0.00 | 3A4 |
| ATOM | 1713 | CB | ASP | 262 | 60.392 | 7.859 | 19.674 | 1.00 | 0.00 | 3A4 |
| ATOM | 1714 | CG | ASP | 262 | 59.293 | 7.671 | 20.735 | 1.00 | 0.00 | 3A4 |
| ATOM | 1715 | OD1 | ASP | 262 | 59.392 | 6.700 | 21.533 | 1.00 | 0.00 | 3A4 |
| ATOM | 1716 | OD2 | ASP | 262 | 58.341 | 8.497 | 20.759 | 1.00 | 0.00 | 3A4 |
| ATOM | 1717 | C | ASP | 262 | 60.828 | 8.011 | 17.175 | 1.00 | 0.00 | 3A4 |
| ATOM | 1718 | O | ASP | 262 | 62.015 | 8.125 | 17.477 | 1.00 | 0.00 | 3A4 |
| ATOM | 1719 | N | THR | 263 | 60.385 | 8.087 | 15.889 | 1.00 | 0.00 | 3A4 |
| ATOM | 1720 | CA | THR | 263 | 61.231 | 8.151 | 14.709 | 1.00 | 0.00 | 3A4 |
| ATOM | 1721 | CB | THR | 263 | 61.257 | 6.828 | 13.922 | 1.00 | 0.00 | 3A4 |
| ATOM | 1722 | OG1 | THR | 263 | 59.962 | 6.247 | 13.768 | 1.00 | 0.00 | 3A4 |
| ATOM | 1723 | CG2 | THR | 263 | 62.163 | 5.831 | 14.677 | 1.00 | 0.00 | 3A4 |
| ATOM | 1724 | C | THR | 263 | 60.819 | 9.346 | 13.857 | 1.00 | 0.00 | 3A4 |
| ATOM | 1725 | O | THR | 263 | 61.248 | 10.466 | 14.127 | 1.00 | 0.00 | 3A4 |
| ATOM | 1726 | N | GLN | 264 | 60.017 | 9.120 | 12.773 | 1.00 | 0.00 | 3A4 |
| ATOM | 1727 | CA | GLN | 264 | 59.831 | 10.032 | 11.649 | 1.00 | 0.00 | 3A4 |
| ATOM | 1728 | CB | GLN | 264 | 59.812 | 9.263 | 10.288 | 1.00 | 0.00 | 3A4 |
| ATOM | 1729 | CG | GLN | 264 | 61.018 | 8.324 | 10.094 | 1.00 | 0.00 | 3A4 |
| ATOM | 1730 | CD | GLN | 264 | 60.951 | 7.671 | 8.710 | 1.00 | 0.00 | 3A4 |
| ATOM | 1731 | OE1 | GLN | 264 | 61.775 | 7.968 | 7.846 | 1.00 | 0.00 | 3A4 |
| ATOM | 1732 | NE2 | GLN | 264 | 59.961 | 6.763 | 8.489 | 1.00 | 0.00 | 3A4 |
| ATOM | 1733 | C | GLN | 264 | 58.564 | 10.861 | 11.768 | 1.00 | 0.00 | 3A4 |
| ATOM | 1734 | O | GLN | 264 | 57.907 | 10.891 | 12.808 | 1.00 | 0.00 | 3A4 |
| ATOM | 1735 | N | LYS | 265 | 58.214 | 11.556 | 10.645 | 1.00 | 0.00 | 3A4 |
| ATOM | 1736 | CA | LYS | 265 | 57.091 | 12.459 | 10.453 | 1.00 | 0.00 | 3A4 |
| ATOM | 1737 | CB | LYS | 265 | 57.471 | 13.608 | 9.470 | 1.00 | 0.00 | 3A4 |
| ATOM | 1738 | CG | LYS | 265 | 56.414 | 14.703 | 9.209 | 1.00 | 0.00 | 3A4 |
| ATOM | 1739 | CD | LYS | 265 | 55.986 | 15.485 | 10.458 | 1.00 | 0.00 | 3A4 |
| ATOM | 1740 | CE | LYS | 265 | 55.004 | 16.618 | 10.140 | 1.00 | 0.00 | 3A4 |
| ATOM | 1741 | NZ | LYS | 265 | 54.585 | 17.323 | 11.374 | 1.00 | 0.00 | 3A4 |
| ATOM | 1742 | C | LYS | 265 | 55.895 | 11.698 | 9.920 | 1.00 | 0.00 | 3A4 |
| ATOM | 1743 | O | LYS | 265 | 54.757 | 11.957 | 10.308 | 1.00 | 0.00 | 3A4 |
| ATOM | 1744 | N | HIS | 266 | 56.161 | 10.712 | 9.017 | 1.00 | 0.00 | 3A4 |
| ATOM | 1745 | CA | HIS | 266 | 55.192 | 9.789 | 8.453 | 1.00 | 0.00 | 3A4 |
| ATOM | 1746 | ND1 | HIS | 266 | 53.213 | 11.484 | 6.555 | 1.00 | 0.00 | 3A4 |
| ATOM | 1747 | CG | HIS | 266 | 54.551 | 11.168 | 6.457 | 1.00 | 0.00 | 3A4 |
| ATOM | 1748 | CB | HIS | 266 | 55.138 | 9.857 | 6.906 | 1.00 | 0.00 | 3A4 |
| ATOM | 1749 | NE2 | HIS | 266 | 54.234 | 13.294 | 5.769 | 1.00 | 0.00 | 3A4 |
| ATOM | 1750 | CD2 | HIS | 266 | 55.161 | 12.287 | 5.977 | 1.00 | 0.00 | 3A4 |
| ATOM | 1751 | CE1 | HIS | 266 | 53.080 | 12.764 | 6.128 | 1.00 | 0.00 | 3A4 |
| ATOM | 1752 | C | HIS | 266 | 55.520 | 8.402 | 8.961 | 1.00 | 0.00 | 3A4 |
| ATOM | 1753 | O | HIS | 266 | 55.595 | 7.432 | 8.208 | 1.00 | 0.00 | 3A4 |
| ATOM | 1754 | N | ARG | 267 | 55.681 | 8.319 | 10.306 | 1.00 | 0.00 | 3A4 |
| ATOM | 1755 | CA | ARG | 267 | 55.787 | 7.112 | 11.088 | 1.00 | 0.00 | 3A4 |
| ATOM | 1756 | CB | ARG | 267 | 57.206 | 6.450 | 11.025 | 1.00 | 0.00 | 3A4 |
| ATOM | 1757 | CG | ARG | 267 | 57.342 | 5.024 | 11.602 | 1.00 | 0.00 | 3A4 |
| ATOM | 1758 | CD | ARG | 267 | 56.433 | 3.983 | 10.915 | 1.00 | 0.00 | 3A4 |
| ATOM | 1759 | NE | ARG | 267 | 56.515 | 2.666 | 11.644 | 1.00 | 0.00 | 3A4 |
| ATOM | 1760 | CZ | ARG | 267 | 55.718 | 2.342 | 12.715 | 1.00 | 0.00 | 3A4 |
| ATOM | 1761 | NH1 | ARG | 267 | 55.829 | 1.104 | 13.280 | 1.00 | 0.00 | 3A4 |
| ATOM | 1762 | NH2 | ARG | 267 | 54.819 | 3.229 | 13.235 | 1.00 | 0.00 | 3A4 |
| ATOM | 1763 | C | ARG | 267 | 55.443 | 7.575 | 12.488 | 1.00 | 0.00 | 3A4 |
| ATOM | 1764 | O | ARG | 267 | 55.403 | 8.776 | 12.757 | 1.00 | 0.00 | 3A4 |
| ATOM | 1765 | N | VAL | 268 | 55.180 | 6.600 | 13.409 | 1.00 | 0.00 | 3A4 |
| ATOM | 1766 | CA | VAL | 268 | 54.850 | 6.700 | 14.830 | 1.00 | 0.00 | 3A4 |
| ATOM | 1767 | CB | VAL | 268 | 55.608 | 7.738 | 15.672 | 1.00 | 0.00 | 3A4 |
| ATOM | 1768 | CG1 | VAL | 268 | 55.286 | 7.564 | 17.181 | 1.00 | 0.00 | 3A4 |
| ATOM | 1769 | CG2 | VAL | 268 | 57.124 | 7.555 | 15.459 | 1.00 | 0.00 | 3A4 |
| ATOM | 1770 | C | VAL | 268 | 53.347 | 6.859 | 14.949 | 1.00 | 0.00 | 3A4 |
| ATOM | 1771 | O | VAL | 268 | 52.814 | 7.966 | 14.876 | 1.00 | 0.00 | 3A4 |
| ATOM | 1772 | N | ASP | 269 | 52.650 | 5.703 | 15.128 | 1.00 | 0.00 | 3A4 |
| ATOM | 1773 | CA | ASP | 269 | 51.217 | 5.484 | 15.039 | 1.00 | 0.00 | 3A4 |
| ATOM | 1774 | CB | ASP | 269 | 50.952 | 4.008 | 14.629 | 1.00 | 0.00 | 3A4 |
| ATOM | 1775 | CG | ASP | 269 | 51.584 | 2.963 | 15.562 | 1.00 | 0.00 | 3A4 |
| ATOM | 1776 | OD1 | ASP | 269 | 52.585 | 2.329 | 15.136 | 1.00 | 0.00 | 3A4 |
| ATOM | 1777 | OD2 | ASP | 269 | 51.040 | 2.734 | 16.671 | 1.00 | 0.00 | 3A4 |
| ATOM | 1778 | C | ASP | 269 | 50.513 | 5.855 | 16.322 | 1.00 | 0.00 | 3A4 |
| ATOM | 1779 | O | ASP | 269 | 51.163 | 6.144 | 17.324 | 1.00 | 0.00 | 3A4 |
| ATOM | 1780 | N | PHE | 270 | 49.161 | 5.834 | 16.331 | 1.00 | 0.00 | 3A4 |
| ATOM | 1781 | CA | PHE | 270 | 48.362 | 6.204 | 17.490 | 1.00 | 0.00 | 3A4 |
| ATOM | 1782 | CB | PHE | 270 | 46.855 | 6.258 | 17.148 | 1.00 | 0.00 | 3A4 |
| ATOM | 1783 | CG | PHE | 270 | 45.990 | 6.766 | 18.282 | 1.00 | 0.00 | 3A4 |
| ATOM | 1784 | CD1 | PHE | 270 | 45.280 | 5.854 | 19.081 | 1.00 | 0.00 | 3A4 |
| ATOM | 1785 | CD2 | PHE | 270 | 46.020 | 8.114 | 18.661 | 1.00 | 0.00 | 3A4 |
| ATOM | 1786 | CE1 | PHE | 270 | 44.531 | 6.282 | 20.177 | 1.00 | 0.00 | 3A4 |
| ATOM | 1787 | CE2 | PHE | 270 | 45.309 | 8.546 | 19.789 | 1.00 | 0.00 | 3A4 |
| ATOM | 1788 | CZ | PHE | 270 | 44.542 | 7.634 | 20.522 | 1.00 | 0.00 | 3A4 |
| ATOM | 1789 | C | PHE | 270 | 48.565 | 5.305 | 18.696 | 1.00 | 0.00 | 3A4 |
| ATOM | 1790 | O | PHE | 270 | 48.623 | 5.789 | 19.822 | 1.00 | 0.00 | 3A4 |
| ATOM | 1791 | N | LEU | 271 | 48.707 | 3.980 | 18.488 | 1.00 | 0.00 | 3A4 |
| ATOM | 1792 | CA | LEU | 271 | 48.911 | 3.039 | 19.569 | 1.00 | 0.00 | 3A4 |
| ATOM | 1793 | CB | LEU | 271 | 48.798 | 1.584 | 19.058 | 1.00 | 0.00 | 3A4 |
| ATOM | 1794 | CG | LEU | 271 | 47.377 | 1.034 | 18.852 | 1.00 | 0.00 | 3A4 |
| ATOM | 1795 | CD1 | LEU | 271 | 47.422 | −0.399 | 18.277 | 1.00 | 0.00 | 3A4 |
| ATOM | 1796 | CD2 | LEU | 271 | 46.550 | 1.035 | 20.150 | 1.00 | 0.00 | 3A4 |
| ATOM | 1797 | C | LEU | 271 | 50.247 | 3.247 | 20.287 | 1.00 | 0.00 | 3A4 |
| ATOM | 1798 | O | LEU | 271 | 50.309 | 3.283 | 21.510 | 1.00 | 0.00 | 3A4 |
| ATOM | 1799 | N | GLN | 272 | 51.325 | 3.513 | 19.518 | 1.00 | 0.00 | 3A4 |
| ATOM | 1800 | CA | GLN | 272 | 52.641 | 3.856 | 20.018 | 1.00 | 0.00 | 3A4 |
| ATOM | 1801 | CB | GLN | 272 | 53.651 | 3.973 | 18.871 | 1.00 | 0.00 | 3A4 |
| ATOM | 1802 | CG | GLN | 272 | 53.989 | 2.568 | 18.334 | 1.00 | 0.00 | 3A4 |
| ATOM | 1803 | CD | GLN | 272 | 54.908 | 2.656 | 17.119 | 1.00 | 0.00 | 3A4 |
| ATOM | 1804 | OE1 | GLN | 272 | 55.225 | 3.735 | 16.626 | 1.00 | 0.00 | 3A4 |
| ATOM | 1805 | NE2 | GLN | 272 | 55.347 | 1.475 | 16.611 | 1.00 | 0.00 | 3A4 |
| ATOM | 1806 | C | GLN | 272 | 52.646 | 5.136 | 20.813 | 1.00 | 0.00 | 3A4 |
| ATOM | 1807 | O | GLN | 272 | 53.254 | 5.214 | 21.874 | 1.00 | 0.00 | 3A4 |
| ATOM | 1808 | N | LEU | 273 | 51.883 | 6.155 | 20.356 | 1.00 | 0.00 | 3A4 |
| ATOM | 1809 | CA | LEU | 273 | 51.695 | 7.415 | 21.048 | 1.00 | 0.00 | 3A4 |
| ATOM | 1810 | CB | LEU | 273 | 50.926 | 8.433 | 20.166 | 1.00 | 0.00 | 3A4 |
| ATOM | 1811 | CG | LEU | 273 | 51.785 | 8.888 | 18.948 | 1.00 | 0.00 | 3A4 |
| ATOM | 1812 | CD1 | LEU | 273 | 50.959 | 9.439 | 17.771 | 1.00 | 0.00 | 3A4 |
| ATOM | 1813 | CD2 | LEU | 273 | 52.899 | 9.882 | 19.332 | 1.00 | 0.00 | 3A4 |
| ATOM | 1814 | C | LEU | 273 | 50.983 | 7.274 | 22.346 | 1.00 | 0.00 | 3A4 |
| ATOM | 1815 | O | LEU | 273 | 51.365 | 7.895 | 23.329 | 1.00 | 0.00 | 3A4 |
| ATOM | 1816 | N | MET | 274 | 49.964 | 6.395 | 22.412 | 1.00 | 0.00 | 3A4 |
| ATOM | 1817 | CA | MET | 274 | 49.244 | 6.073 | 23.621 | 1.00 | 0.00 | 3A4 |
| ATOM | 1818 | CB | MET | 274 | 47.977 | 5.254 | 23.316 | 1.00 | 0.00 | 3A4 |
| ATOM | 1819 | CG | MET | 274 | 46.828 | 6.090 | 22.751 | 1.00 | 0.00 | 3A4 |
| ATOM | 1820 | SD | MET | 274 | 45.317 | 6.067 | 23.770 | 1.00 | 0.00 | 3A4 |
| ATOM | 1821 | CE | MET | 274 | 45.991 | 6.940 | 25.218 | 1.00 | 0.00 | 3A4 |
| ATOM | 1822 | C | MET | 274 | 50.109 | 5.325 | 24.619 | 1.00 | 0.00 | 3A4 |
| ATOM | 1823 | O | MET | 274 | 50.038 | 5.567 | 25.819 | 1.00 | 0.00 | 3A4 |
| ATOM | 1824 | N | ILE | 275 | 50.988 | 4.417 | 24.131 | 1.00 | 0.00 | 3A4 |
| ATOM | 1825 | CA | ILE | 275 | 51.851 | 3.564 | 24.931 | 1.00 | 0.00 | 3A4 |
| ATOM | 1826 | CB | ILE | 275 | 52.467 | 2.441 | 24.078 | 1.00 | 0.00 | 3A4 |
| ATOM | 1827 | CG2 | ILE | 275 | 53.965 | 2.545 | 23.648 | 1.00 | 0.00 | 3A4 |
| ATOM | 1828 | CG1 | ILE | 275 | 52.209 | 1.038 | 24.643 | 1.00 | 0.00 | 3A4 |
| ATOM | 1829 | CD | ILE | 275 | 51.787 | 0.081 | 23.536 | 1.00 | 0.00 | 3A4 |
| ATOM | 1830 | C | ILE | 275 | 52.925 | 4.329 | 25.660 | 1.00 | 0.00 | 3A4 |
| ATOM | 1831 | O | ILE | 275 | 53.323 | 3.975 | 26.764 | 1.00 | 0.00 | 3A4 |
| ATOM | 1832 | N | ASP | 276 | 53.392 | 5.425 | 25.033 | 1.00 | 0.00 | 3A4 |
| ATOM | 1633 | CA | ASP | 276 | 54.488 | 6.233 | 25.491 | 1.00 | 0.00 | 3A4 |
| ATOM | 1834 | CB | ASP | 276 | 55.375 | 6.635 | 24.263 | 1.00 | 0.00 | 3A4 |
| ATOM | 1835 | CG | ASP | 276 | 56.766 | 7.177 | 24.648 | 1.00 | 0.00 | 3A4 |
| ATOM | 1836 | OD1 | ASP | 276 | 57.527 | 6.432 | 25.323 | 1.00 | 0.00 | 3A4 |
| ATOM | 1837 | OD2 | ASP | 276 | 57.080 | 8.336 | 24.266 | 1.00 | 0.00 | 3A4 |
| ATOM | 1838 | C | ASP | 276 | 53.994 | 7.467 | 26.229 | 1.00 | 0.00 | 3A4 |
| ATOM | 1839 | O | ASP | 276 | 54.738 | 8.023 | 27.029 | 1.00 | 0.00 | 3A4 |
| ATOM | 1840 | N | SER | 277 | 52.734 | 7.918 | 25.959 | 1.00 | 0.00 | 3A4 |
| ATOM | 1841 | CA | SER | 277 | 51.962 | 9.005 | 26.574 | 1.00 | 0.00 | 3A4 |
| ATOM | 1842 | CB | SER | 277 | 51.494 | 8.689 | 28.034 | 1.00 | 0.00 | 3A4 |
| ATOM | 1843 | OG | SER | 277 | 52.536 | 8.498 | 28.989 | 1.00 | 0.00 | 3A4 |
| ATOM | 1844 | C | SER | 277 | 52.586 | 10.401 | 26.477 | 1.00 | 0.00 | 3A4 |
| ATOM | 1845 | O | SER | 277 | 53.547 | 10.719 | 27.177 | 1.00 | 0.00 | 3A4 |
| ATOM | 1846 | N | GLN | 278 | 52.027 | 11.259 | 25.584 | 1.00 | 0.00 | 3A4 |
| ATOM | 1847 | CA | GLN | 278 | 52.576 | 12.557 | 25.230 | 1.00 | 0.00 | 3A4 |
| ATOM | 1848 | CB | GLN | 278 | 53.079 | 12.585 | 23.748 | 1.00 | 0.00 | 3A4 |
| ATOM | 1849 | CG | GLN | 278 | 52.289 | 11.752 | 22.708 | 1.00 | 0.00 | 3A4 |
| ATOM | 1850 | CD | GLN | 278 | 50.896 | 12.317 | 22.436 | 1.00 | 0.00 | 3A4 |
| ATOM | 1851 | OE1 | GLN | 278 | 49.896 | 11.703 | 22.807 | 1.00 | 0.00 | 3A4 |
| ATOM | 1852 | NE2 | GLN | 278 | 50.821 | 13.500 | 21.767 | 1.00 | 0.00 | 3A4 |
| ATOM | 1853 | C | GLN | 278 | 51.568 | 13.647 | 25.534 | 1.00 | 0.00 | 3A4 |
| ATOM | 1854 | O | GLN | 278 | 51.306 | 14.524 | 24.712 | 1.00 | 0.00 | 3A4 |
| ATOM | 1855 | N | ASN | 279 | 51.016 | 13.632 | 26.775 | 1.00 | 0.00 | 3A4 |
| ATOM | 1856 | CA | ASN | 279 | 50.193 | 14.697 | 27.310 | 1.00 | 0.00 | 3A4 |
| ATOM | 1857 | CB | ASN | 279 | 48.716 | 14.690 | 26.781 | 1.00 | 0.00 | 3A4 |
| ATOM | 1858 | CG | ASN | 279 | 47.973 | 15.999 | 27.107 | 1.00 | 0.00 | 3A4 |
| ATOM | 1859 | OD1 | ASN | 279 | 47.305 | 16.089 | 28.136 | 1.00 | 0.00 | 3A4 |
| ATOM | 1860 | ND2 | ASN | 279 | 48.086 | 17.026 | 26.221 | 1.00 | 0.00 | 3A4 |
| ATOM | 1861 | C | ASN | 279 | 50.239 | 14.505 | 28.808 | 1.00 | 0.00 | 3A4 |
| ATOM | 1862 | O | ASN | 279 | 50.626 | 15.409 | 29.546 | 1.00 | 0.00 | 3A4 |
| ATOM | 1863 | N | SER | 280 | 49.821 | 13.295 | 29.277 | 1.00 | 0.00 | 3A4 |
| ATOM | 1864 | CA | SER | 280 | 49.736 | 12.901 | 30.674 | 1.00 | 0.00 | 3A4 |
| ATOM | 1865 | CB | SER | 280 | 48.334 | 12.330 | 31.056 | 1.00 | 0.00 | 3A4 |
| ATOM | 1866 | OG | SER | 280 | 47.313 | 13.268 | 30.740 | 1.00 | 0.00 | 3A4 |
| ATOM | 1867 | C | SER | 280 | 50.810 | 11.870 | 30.942 | 1.00 | 0.00 | 3A4 |
| ATOM | 1868 | O | SER | 280 | 51.635 | 11.569 | 30.079 | 1.00 | 0.00 | 3A4 |
| ATOM | 1869 | N | LYS | 281 | 50.813 | 11.317 | 32.183 | 1.00 | 0.00 | 3A4 |
| ATOM | 1870 | CA | LYS | 281 | 51.810 | 10.412 | 32.724 | 1.00 | 0.00 | 3A4 |
| ATOM | 1871 | CB | LYS | 281 | 52.318 | 10.863 | 34.135 | 1.00 | 0.00 | 3A4 |
| ATOM | 1672 | CG | LYS | 281 | 51.368 | 10.740 | 35.357 | 1.00 | 0.00 | 3A4 |
| ATOM | 1873 | CD | LYS | 281 | 50.099 | 11.612 | 35.328 | 1.00 | 0.00 | 3A4 |
| ATOM | 1874 | CE | LYS | 281 | 49.241 | 11.508 | 36.598 | 1.00 | 0.00 | 3A4 |
| ATOM | 1875 | NZ | LYS | 281 | 48.695 | 10.139 | 36.770 | 1.00 | 0.00 | 3A4 |
| ATOM | 1876 | C | LYS | 281 | 51.259 | 9.009 | 32.800 | 1.00 | 0.00 | 3A4 |
| ATOM | 1877 | O | LYS | 281 | 50.051 | 8.792 | 32.725 | 1.00 | 0.00 | 3A4 |
| ATOM | 1878 | N | GLU | 282 | 52.179 | 8.025 | 33.003 | 1.00 | 0.00 | 3A4 |
| ATOM | 1879 | CA | GLU | 282 | 51.902 | 6.623 | 33.252 | 1.00 | 0.00 | 3A4 |
| ATOM | 1880 | CB | GLU | 282 | 52.839 | 5.684 | 32.439 | 1.00 | 0.00 | 3A4 |
| ATOM | 1881 | CG | GLU | 282 | 54.355 | 5.905 | 32.633 | 1.00 | 0.00 | 3A4 |
| ATOM | 1882 | CD | GLU | 282 | 55.125 | 5.055 | 31.617 | 1.00 | 0.00 | 3A4 |
| ATOM | 1883 | OE1 | GLU | 282 | 54.983 | 5.322 | 30.393 | 1.00 | 0.00 | 3A4 |
| ATOM | 1884 | OE2 | GLU | 282 | 55.866 | 4.133 | 32.049 | 1.00 | 0.00 | 3A4 |
| ATOM | 1885 | C | GLU | 282 | 52.026 | 6.378 | 34.739 | 1.00 | 0.00 | 3A4 |
| ATOM | 1886 | O | GLU | 282 | 52.842 | 7.002 | 35.416 | 1.00 | 0.00 | 3A4 |
| ATOM | 1887 | N | THR | 283 | 51.169 | 5.468 | 35.281 | 1.00 | 0.00 | 3A4 |
| ATOM | 1888 | CA | THR | 283 | 50.949 | 5.262 | 36.708 | 1.00 | 0.00 | 3A4 |
| ATOM | 1889 | CB | THR | 283 | 49.462 | 5.076 | 37.037 | 1.00 | 0.00 | 3A4 |
| ATOM | 1890 | OG1 | THR | 283 | 48.843 | 4.071 | 36.236 | 1.00 | 0.00 | 3A4 |
| ATOM | 1891 | CG2 | THR | 283 | 48.746 | 6.425 | 36.797 | 1.00 | 0.00 | 3A4 |
| ATOM | 1892 | C | THR | 283 | 51.786 | 4.117 | 37.266 | 1.00 | 0.00 | 3A4 |
| ATOM | 1893 | O | THR | 283 | 51.843 | 3.935 | 38.481 | 1.00 | 0.00 | 3A4 |
| ATOM | 1894 | N | GLU | 284 | 52.457 | 3.329 | 36.385 | 1.00 | 0.00 | 3A4 |
| ATOM | 1895 | CA | GLU | 284 | 53.336 | 2.253 | 36.792 | 1.00 | 0.00 | 3A4 |
| ATOM | 1896 | CB | GLU | 284 | 52.583 | 0.907 | 37.025 | 1.00 | 0.00 | 3A4 |
| ATOM | 1897 | CG | GLU | 284 | 53.427 | −0.211 | 37.670 | 1.00 | 0.00 | 3A4 |
| ATOM | 1898 | CD | GLU | 284 | 52.553 | −1.452 | 37.873 | 1.00 | 0.00 | 3A4 |
| ATOM | 1899 | OE1 | GLU | 284 | 52.075 | −2.014 | 36.851 | 1.00 | 0.00 | 3A4 |
| ATOM | 1900 | OE2 | GLU | 284 | 52.352 | −1.855 | 39.051 | 1.00 | 0.00 | 3A4 |
| ATOM | 1901 | C | GLU | 284 | 54.393 | 2.152 | 35.719 | 1.00 | 0.00 | 3A4 |
| ATOM | 1902 | O | GLU | 284 | 55.397 | 2.861 | 35.770 | 1.00 | 0.00 | 3A4 |
| ATOM | 1903 | N | SER | 285 | 54.186 | 1.256 | 34.721 | 1.00 | 0.00 | 3A4 |
| ATOM | 1904 | CA | SER | 285 | 55.139 | 0.913 | 33.669 | 1.00 | 0.00 | 3A4 |
| ATOM | 1905 | CB | SER | 285 | 56.281 | −0.008 | 34.108 | 1.00 | 0.00 | 3A4 |
| ATOM | 1906 | OG | SER | 285 | 55.800 | −1.171 | 34.781 | 1.00 | 0.00 | 3A4 |
| ATOM | 1907 | C | SER | 285 | 54.331 | 0.438 | 32.510 | 1.00 | 0.00 | 3A4 |
| ATOM | 1908 | O | SER | 285 | 54.522 | −0.694 | 32.066 | 1.00 | 0.00 | 3A4 |
| ATOM | 1909 | N | HIS | 286 | 53.396 | 1.275 | 31.998 | 1.00 | 0.00 | 3A4 |
| ATOM | 1910 | CA | HIS | 286 | 52.522 | 0.926 | 30.901 | 1.00 | 0.00 | 3A4 |
| ATOM | 1911 | ND1 | HIS | 286 | 49.824 | 1.672 | 32.816 | 1.00 | 0.00 | 3A4 |
| ATOM | 1912 | CG | HIS | 286 | 50.594 | 0.542 | 32.645 | 1.00 | 0.00 | 3A4 |
| ATOM | 1913 | CB | HIS | 286 | 51.264 | 0.113 | 31.356 | 1.00 | 0.00 | 3A4 |
| ATOM | 1914 | NE2 | HIS | 286 | 49.782 | 0.592 | 34.753 | 1.00 | 0.00 | 3A4 |
| ATOM | 1915 | CD2 | HIS | 286 | 50.557 | −0.106 | 33.843 | 1.00 | 0.00 | 3A4 |
| ATOM | 1916 | CE1 | HIS | 286 | 49.366 | 1.652 | 34.090 | 1.00 | 0.00 | 3A4 |
| ATOM | 1917 | C | HIS | 286 | 52.192 | 2.222 | 30.200 | 1.00 | 0.00 | 3A4 |
| ATOM | 1918 | O | HIS | 286 | 53.089 | 2.937 | 29.758 | 1.00 | 0.00 | 3A4 |
| ATOM | 1919 | N | LYS | 287 | 50.878 | 2.537 | 30.069 | 1.00 | 0.00 | 3A4 |
| ATOM | 1920 | CA | LYS | 287 | 50.317 | 3.652 | 29.338 | 1.00 | 0.00 | 3A4 |
| ATOM | 1921 | CB | LYS | 287 | 49.136 | 3.180 | 28.453 | 1.00 | 0.00 | 3A4 |
| ATOM | 1922 | CG | LYS | 287 | 49.553 | 2.209 | 27.349 | 1.00 | 0.00 | 3A4 |
| ATOM | 1923 | CD | LYS | 287 | 48.717 | 2.529 | 26.095 | 1.00 | 0.00 | 3A4 |
| ATOM | 1924 | CE | LYS | 287 | 48.931 | 1.795 | 24.736 | 1.00 | 0.00 | 3A4 |
| ATOM | 1925 | NZ | LYS | 287 | 47.885 | 2.079 | 23.732 | 1.00 | 0.00 | 3A4 |
| ATOM | 1926 | C | LYS | 287 | 49.794 | 4.698 | 30.287 | 1.00 | 0.00 | 3A4 |
| ATOM | 1927 | O | LYS | 287 | 49.872 | 4.548 | 31.504 | 1.00 | 0.00 | 3A4 |
| ATOM | 1928 | N | ALA | 288 | 49.185 | 5.771 | 29.701 | 1.00 | 0.00 | 3A4 |
| ATOM | 1929 | CA | ALA | 288 | 48.374 | 6.783 | 30.350 | 1.00 | 0.00 | 3A4 |
| ATOM | 1930 | CB | ALA | 288 | 48.615 | 8.202 | 29.810 | 1.00 | 0.00 | 3A4 |
| ATOM | 1931 | C | ALA | 288 | 46.906 | 6.403 | 30.192 | 1.00 | 0.00 | 3A4 |
| ATOM | 1932 | O | ALA | 288 | 46.333 | 6.618 | 29.125 | 1.00 | 0.00 | 3A4 |
| ATOM | 1933 | N | LEU | 289 | 46.245 | 5.810 | 31.226 | 1.00 | 0.00 | 3A4 |
| ATOM | 1934 | CA | LEU | 289 | 46.774 | 5.489 | 32.535 | 1.00 | 0.00 | 3A4 |
| ATOM | 1935 | CB | LEU | 289 | 46.349 | 6.484 | 33.668 | 1.00 | 0.00 | 3A4 |
| ATOM | 1936 | CG | LEU | 289 | 44.839 | 6.740 | 33.920 | 1.00 | 0.00 | 3A4 |
| ATOM | 1937 | CD1 | LEU | 289 | 44.604 | 7.179 | 35.378 | 1.00 | 0.00 | 3A4 |
| ATOM | 1938 | CD2 | LEU | 289 | 44.221 | 7.776 | 32.951 | 1.00 | 0.00 | 3A4 |
| ATOM | 1939 | C | LEU | 289 | 46.475 | 4.056 | 32.878 | 1.00 | 0.00 | 3A4 |
| ATOM | 1940 | O | LEU | 289 | 46.614 | 3.704 | 34.048 | 1.00 | 0.00 | 3A4 |
| ATOM | 1941 | N | SER | 290 | 46.131 | 3.176 | 31.880 | 1.00 | 0.00 | 3A4 |
| ATOM | 1942 | CA | SER | 290 | 46.112 | 1.765 | 32.148 | 1.00 | 0.00 | 3A4 |
| ATOM | 1943 | CB | SER | 290 | 45.026 | 1.325 | 33.179 | 1.00 | 0.00 | 3A4 |
| ATOM | 1944 | OG | SER | 290 | 43.728 | 1.835 | 32.895 | 1.00 | 0.00 | 3A4 |
| ATOM | 1945 | C | SER | 290 | 45.931 | 1.008 | 30.867 | 1.00 | 0.00 | 3A4 |
| ATOM | 1946 | O | SER | 290 | 45.432 | 1.483 | 29.847 | 1.00 | 0.00 | 3A4 |
| ATOM | 1947 | N | ASP | 291 | 46.293 | −0.287 | 30.944 | 1.00 | 0.00 | 3A4 |
| ATOM | 1948 | CA | ASP | 291 | 46.226 | −1.223 | 29.856 | 1.00 | 0.00 | 3A4 |
| ATOM | 1949 | CB | ASP | 291 | 46.894 | −2.555 | 30.218 | 1.00 | 0.00 | 3A4 |
| ATOM | 1950 | CG | ASP | 291 | 48.385 | −2.331 | 30.513 | 1.00 | 0.00 | 3A4 |
| ATOM | 1951 | OD1 | ASP | 291 | 49.127 | −1.982 | 29.555 | 1.00 | 0.00 | 3A4 |
| ATOM | 1952 | OD2 | ASP | 291 | 48.800 | −2.508 | 31.690 | 1.00 | 0.00 | 3A4 |
| ATOM | 1953 | C | ASP | 291 | 44.820 | −1.495 | 29.390 | 1.00 | 0.00 | 3A4 |
| ATOM | 1954 | O | ASP | 291 | 44.578 | −1.735 | 28.227 | 1.00 | 0.00 | 3A4 |
| ATOM | 1955 | N | LEU | 292 | 43.831 | −1.426 | 30.296 | 1.00 | 0.00 | 3A4 |
| ATOM | 1956 | CA | LEU | 292 | 42.433 | −1.624 | 29.999 | 1.00 | 0.00 | 3A4 |
| ATOM | 1957 | CB | LEU | 292 | 41.659 | −1.848 | 31.312 | 1.00 | 0.00 | 3A4 |
| ATOM | 1958 | CG | LEU | 292 | 41.910 | −3.218 | 31.978 | 1.00 | 0.00 | 3A4 |
| ATOM | 1959 | CD1 | LEU | 292 | 41.502 | −3.197 | 33.463 | 1.00 | 0.00 | 3A4 |
| ATOM | 1960 | CD2 | LEU | 292 | 41.193 | −4.362 | 31.230 | 1.00 | 0.00 | 3A4 |
| ATOM | 1961 | C | LEU | 292 | 41.827 | −0.445 | 29.268 | 1.00 | 0.00 | 3A4 |
| ATOM | 1962 | O | LEU | 292 | 41.078 | −0.602 | 28.309 | 1.00 | 0.00 | 3A4 |
| ATOM | 1963 | N | GLU | 293 | 42.216 | 0.784 | 29.669 | 1.00 | 0.00 | 3A4 |
| ATOM | 1964 | CA | GLU | 293 | 41.794 | 2.025 | 29.064 | 1.00 | 0.00 | 3A4 |
| ATOM | 1965 | CB | GLU | 293 | 42.258 | 3.233 | 29.892 | 1.00 | 0.00 | 3A4 |
| ATOM | 1966 | CG | GLU | 293 | 41.403 | 3.396 | 31.163 | 1.00 | 0.00 | 3A4 |
| ATOM | 1967 | CD | GLU | 293 | 41.950 | 4.520 | 32.046 | 1.00 | 0.00 | 3A4 |
| ATOM | 1968 | OE1 | GLU | 293 | 41.285 | 4.842 | 33.067 | 1.00 | 0.00 | 3A4 |
| ATOM | 1969 | OE2 | GLU | 293 | 43.037 | 5.066 | 31.722 | 1.00 | 0.00 | 3A4 |
| ATOM | 1970 | C | GLU | 293 | 42.286 | 2.186 | 27.666 | 1.00 | 0.00 | 3A4 |
| ATOM | 1971 | O | GLU | 293 | 41.540 | 2.607 | 26.793 | 1.00 | 0.00 | 3A4 |
| ATOM | 1972 | N | LEU | 294 | 43.547 | 1.769 | 27.420 | 1.00 | 0.00 | 3A4 |
| ATOM | 1973 | CA | LEU | 294 | 44.175 | 1.802 | 26.122 | 1.00 | 0.00 | 3A4 |
| ATOM | 1974 | CB | LEU | 294 | 45.633 | 1.380 | 26.208 | 1.00 | 0.00 | 3A4 |
| ATOM | 1975 | CG | LEU | 294 | 46.091 | −0.086 | 26.051 | 1.00 | 0.00 | 3A4 |
| ATOM | 1976 | CD1 | LEU | 294 | 46.203 | −0.637 | 24.643 | 1.00 | 0.00 | 3A4 |
| ATOM | 1977 | CD2 | LEU | 294 | 47.365 | −0.436 | 26.810 | 1.00 | 0.00 | 3A4 |
| ATOM | 1978 | C | LEU | 294 | 43.443 | 0.944 | 25.134 | 1.00 | 0.00 | 3A4 |
| ATOM | 1979 | O | LEU | 294 | 43.172 | 1.361 | 24.020 | 1.00 | 0.00 | 3A4 |
| ATOM | 1980 | N | VAL | 295 | 43.044 | −0.282 | 25.551 | 1.00 | 0.00 | 3A4 |
| ATOM | 1981 | CA | VAL | 295 | 42.358 | −1.242 | 24.721 | 1.00 | 0.00 | 3A4 |
| ATOM | 1982 | CB | VAL | 295 | 42.271 | −2.600 | 25.380 | 1.00 | 0.00 | 3A4 |
| ATOM | 1983 | CG1 | VAL | 295 | 41.397 | −3.622 | 24.599 | 1.00 | 0.00 | 3A4 |
| ATOM | 1984 | CG2 | VAL | 295 | 43.701 | −3.178 | 25.429 | 1.00 | 0.00 | 3A4 |
| ATOM | 1985 | C | VAL | 295 | 40.980 | −0.762 | 24.357 | 1.00 | 0.00 | 3A4 |
| ATOM | 1986 | O | VAL | 295 | 40.584 | −0.846 | 23.207 | 1.00 | 0.00 | 3A4 |
| ATOM | 1987 | N | ALA | 296 | 40.252 | −0.146 | 25.306 | 1.00 | 0.00 | 3A4 |
| ATOM | 1988 | CA | ALA | 296 | 38.945 | 0.439 | 25.074 | 1.00 | 0.00 | 3A4 |
| ATOM | 1989 | CB | ALA | 296 | 38.344 | 0.902 | 26.408 | 1.00 | 0.00 | 3A4 |
| ATOM | 1990 | C | ALA | 296 | 38.944 | 1.619 | 24.132 | 1.00 | 0.00 | 3A4 |
| ATOM | 1991 | O | ALA | 296 | 38.032 | 1.797 | 23.342 | 1.00 | 0.00 | 3A4 |
| ATOM | 1992 | N | GLN | 297 | 40.018 | 2.430 | 24.170 | 1.00 | 0.00 | 3A4 |
| ATOM | 1993 | CA | GLN | 297 | 40.228 | 3.546 | 23.284 | 1.00 | 0.00 | 3A4 |
| ATOM | 1994 | CB | GLN | 297 | 41.379 | 4.436 | 23.773 | 1.00 | 0.00 | 3A4 |
| ATOM | 1995 | CG | GLN | 297 | 40.914 | 5.233 | 25.008 | 1.00 | 0.00 | 3A4 |
| ATOM | 1996 | CD | GLN | 297 | 42.099 | 5.887 | 25.715 | 1.00 | 0.00 | 3A4 |
| ATOM | 1997 | OE1 | GLN | 297 | 43.052 | 5.222 | 26.114 | 1.00 | 0.00 | 3A4 |
| ATOM | 1998 | NE2 | GLN | 297 | 42.034 | 7.228 | 25.902 | 1.00 | 0.00 | 3A4 |
| ATOM | 1999 | C | GLN | 297 | 40.505 | 3.108 | 21.882 | 1.00 | 0.00 | 3A4 |
| ATOM | 2000 | O | GLN | 297 | 39.872 | 3.572 | 20.943 | 1.00 | 0.00 | 3A4 |
| ATOM | 2001 | N | SER | 298 | 41.403 | 2.117 | 21.715 | 1.00 | 0.00 | 3A4 |
| ATOM | 2002 | CA | SER | 298 | 41.744 | 1.546 | 20.434 | 1.00 | 0.00 | 3A4 |
| ATOM | 2003 | CB | SER | 298 | 42.925 | 0.563 | 20.529 | 1.00 | 0.00 | 3A4 |
| ATOM | 2004 | OG | SER | 298 | 42.771 | −0.519 | 21.435 | 1.00 | 0.00 | 3A4 |
| ATOM | 2005 | C | SER | 298 | 40.584 | 0.872 | 19.755 | 1.00 | 0.00 | 3A4 |
| ATOM | 2006 | O | SER | 298 | 40.435 | 0.977 | 18.546 | 1.00 | 0.00 | 3A4 |
| ATOM | 2007 | N | ILE | 299 | 39.688 | 0.228 | 20.542 | 1.00 | 0.00 | 3A4 |
| ATOM | 2008 | CA | ILE | 299 | 38.438 | −0.363 | 20.098 | 1.00 | 0.00 | 3A4 |
| ATOM | 2009 | CB | ILE | 299 | 37.670 | −1.032 | 21.291 | 1.00 | 0.00 | 3A4 |
| ATOM | 2010 | CG2 | ILE | 299 | 36.134 | −1.268 | 21.098 | 1.00 | 0.00 | 3A4 |
| ATOM | 2011 | CG1 | ILE | 299 | 38.328 | −2.333 | 21.803 | 1.00 | 0.00 | 3A4 |
| ATOM | 2012 | CD | ILE | 299 | 37.787 | −3.619 | 21.185 | 1.00 | 0.00 | 3A4 |
| ATOM | 2013 | C | ILE | 299 | 37.515 | 0.677 | 19.534 | 1.00 | 0.00 | 3A4 |
| ATOM | 2014 | O | ILE | 299 | 36.949 | 0.525 | 18.457 | 1.00 | 0.00 | 3A4 |
| ATOM | 2015 | N | ILE | 300 | 37.374 | 1.799 | 20.268 | 1.00 | 0.00 | 3A4 |
| ATOM | 2016 | CA | ILE | 300 | 36.494 | 2.883 | 19.921 | 1.00 | 0.00 | 3A4 |
| ATOM | 2017 | CB | ILE | 300 | 36.290 | 3.798 | 21.113 | 1.00 | 0.00 | 3A4 |
| ATOM | 2018 | CG2 | ILE | 300 | 36.292 | 5.340 | 20.897 | 1.00 | 0.00 | 3A4 |
| ATOM | 2019 | CG1 | ILE | 300 | 34.906 | 3.296 | 21.637 | 1.00 | 0.00 | 3A4 |
| ATOM | 2020 | CD | ILE | 300 | 34.503 | 3.642 | 23.050 | 1.00 | 0.00 | 3A4 |
| ATOM | 2021 | C | ILE | 300 | 36.962 | 3.618 | 18.712 | 1.00 | 0.00 | 3A4 |
| ATOM | 2022 | O | ILE | 300 | 36.161 | 3.994 | 17.872 | 1.00 | 0.00 | 3A4 |
| ATOM | 2023 | N | PHE | 301 | 38.279 | 3.774 | 18.515 | 1.00 | 0.00 | 3A4 |
| ATOM | 2024 | CA | PHE | 301 | 38.819 | 4.525 | 17.406 | 1.00 | 0.00 | 3A4 |
| ATOM | 2025 | CB | PHE | 301 | 40.332 | 4.745 | 17.663 | 1.00 | 0.00 | 3A4 |
| ATOM | 2026 | CG | PHE | 301 | 40.520 | 5.958 | 18.535 | 1.00 | 0.00 | 3A4 |
| ATOM | 2027 | CD1 | PHE | 301 | 41.157 | 5.931 | 19.784 | 1.00 | 0.00 | 3A4 |
| ATOM | 2028 | CD2 | PHE | 301 | 39.913 | 7.144 | 18.106 | 1.00 | 0.00 | 3A4 |
| ATOM | 2029 | CE1 | PHE | 301 | 41.032 | 7.015 | 20.660 | 1.00 | 0.00 | 3A4 |
| ATOM | 2030 | CE2 | PHE | 301 | 39.784 | 8.213 | 18.969 | 1.00 | 0.00 | 3A4 |
| ATOM | 2031 | CZ | PHE | 301 | 40.300 | 8.142 | 20.259 | 1.00 | 0.00 | 3A4 |
| ATOM | 2032 | C | PHE | 301 | 38.633 | 3.743 | 16.111 | 1.00 | 0.00 | 3A4 |
| ATOM | 2033 | O | PHE | 301 | 38.332 | 4.306 | 15.062 | 1.00 | 0.00 | 3A4 |
| ATOM | 2034 | N | ILE | 302 | 38.742 | 2.401 | 16.178 | 1.00 | 0.00 | 3A4 |
| ATOM | 2035 | CA | ILE | 302 | 38.511 | 1.491 | 15.076 | 1.00 | 0.00 | 3A4 |
| ATOM | 2036 | CB | ILE | 302 | 39.005 | 0.104 | 15.470 | 1.00 | 0.00 | 3A4 |
| ATOM | 2037 | CG2 | ILE | 302 | 38.482 | −1.056 | 14.570 | 1.00 | 0.00 | 3A4 |
| ATOM | 2038 | CG1 | ILE | 302 | 40.563 | 0.105 | 15.527 | 1.00 | 0.00 | 3A4 |
| ATOM | 2039 | CD | ILE | 302 | 41.293 | 0.195 | 14.178 | 1.00 | 0.00 | 3A4 |
| ATOM | 2040 | C | ILE | 302 | 37.054 | 1.462 | 14.656 | 1.00 | 0.00 | 3A4 |
| ATOM | 2041 | O | ILE | 302 | 36.732 | 1.682 | 13.491 | 1.00 | 0.00 | 3A4 |
| ATOM | 2042 | N | PHE | 303 | 36.126 | 1.231 | 15.610 | 1.00 | 0.00 | 3A4 |
| ATOM | 2043 | CA | PHE | 303 | 34.711 | 1.090 | 15.324 | 1.00 | 0.00 | 3A4 |
| ATOM | 2044 | CB | PHE | 303 | 34.008 | 0.159 | 16.395 | 1.00 | 0.00 | 3A4 |
| ATOM | 2045 | CG | PHE | 303 | 33.072 | 0.735 | 17.449 | 1.00 | 0.00 | 3A4 |
| ATOM | 2046 | CD1 | PHE | 303 | 31.783 | 1.209 | 17.127 | 1.00 | 0.00 | 3A4 |
| ATOM | 2047 | CD2 | PHE | 303 | 33.443 | 0.706 | 18.808 | 1.00 | 0.00 | 3A4 |
| ATOM | 2048 | CE1 | PHE | 303 | 30.911 | 1.674 | 18.121 | 1.00 | 0.00 | 3A4 |
| ATOM | 2049 | CE2 | PHE | 303 | 32.572 | 1.155 | 19.810 | 1.00 | 0.00 | 3A4 |
| ATOM | 2050 | CZ | PHE | 303 | 31.307 | 1.647 | 19.465 | 1.00 | 0.00 | 3A4 |
| ATOM | 2051 | C | PHE | 303 | 34.016 | 2.407 | 14.993 | 1.00 | 0.00 | 3A4 |
| ATOM | 2052 | O | PHE | 303 | 33.183 | 2.472 | 14.092 | 1.00 | 0.00 | 3A4 |
| ATOM | 2053 | N | ALA | 304 | 34.391 | 3.513 | 15.679 | 1.00 | 0.00 | 3A4 |
| ATOM | 2054 | CA | ALA | 304 | 33.814 | 4.831 | 15.484 | 1.00 | 0.00 | 3A4 |
| ATOM | 2055 | CB | ALA | 304 | 34.124 | 5.826 | 16.638 | 1.00 | 0.00 | 3A4 |
| ATOM | 2056 | C | ALA | 304 | 34.221 | 5.470 | 14.208 | 1.00 | 0.00 | 3A4 |
| ATOM | 2057 | O | ALA | 304 | 33.449 | 6.196 | 13.589 | 1.00 | 0.00 | 3A4 |
| ATOM | 2058 | N | GLY | 305 | 35.466 | 5.191 | 13.787 | 1.00 | 0.00 | 3A4 |
| ATOM | 2059 | CA | GLY | 305 | 36.037 | 5.755 | 12.599 | 1.00 | 0.00 | 3A4 |
| ATOM | 2060 | C | GLY | 305 | 35.766 | 5.044 | 11.304 | 1.00 | 0.00 | 3A4 |
| ATOM | 2061 | O | GLY | 305 | 36.028 | 5.603 | 10.243 | 1.00 | 0.00 | 3A4 |
| ATOM | 2062 | N | TYR | 306 | 35.257 | 3.789 | 11.337 | 1.00 | 0.00 | 3A4 |
| ATOM | 2063 | CA | TYR | 306 | 35.125 | 2.982 | 10.139 | 1.00 | 0.00 | 3A4 |
| ATOM | 2064 | CB | TYR | 306 | 35.531 | 1.484 | 10.389 | 1.00 | 0.00 | 3A4 |
| ATOM | 2065 | CG | TYR | 306 | 35.403 | 0.528 | 9.206 | 1.00 | 0.00 | 3A4 |
| ATOM | 2066 | CD1 | TYR | 306 | 35.378 | 0.950 | 7.858 | 1.00 | 0.00 | 3A4 |
| ATOM | 2067 | CD2 | TYR | 306 | 35.195 | −0.835 | 9.469 | 1.00 | 0.00 | 3A4 |
| ATOM | 2068 | CE1 | TYR | 306 | 35.009 | 0.064 | 6.843 | 1.00 | 0.00 | 3A4 |
| ATOM | 2069 | CE2 | TYR | 306 | 34.885 | −1.738 | 8.441 | 1.00 | 0.00 | 3A4 |
| ATOM | 2070 | CZ | TYR | 306 | 34.766 | −1.281 | 7.125 | 1.00 | 0.00 | 3A4 |
| ATOM | 2071 | OH | TYR | 306 | 34.383 | −2.159 | 6.089 | 1.00 | 0.00 | 3A4 |
| ATOM | 2072 | C | TYR | 306 | 33.741 | 3.132 | 9.572 | 1.00 | 0.00 | 3A4 |
| ATOM | 2073 | O | TYR | 306 | 33.566 | 3.553 | 8.431 | 1.00 | 0.00 | 3A4 |
| ATOM | 2074 | N | GLU | 307 | 32.692 | 2.781 | 10.344 | 1.00 | 0.00 | 3A4 |
| ATOM | 2075 | CA | GLU | 307 | 31.384 | 2.639 | 9.745 | 1.00 | 0.00 | 3A4 |
| ATOM | 2076 | CB | GLU | 307 | 30.517 | 1.553 | 10.437 | 1.00 | 0.00 | 3A4 |
| ATOM | 2077 | CG | GLU | 307 | 31.119 | 0.127 | 10.329 | 1.00 | 0.00 | 3A4 |
| ATOM | 2078 | CD | GLU | 307 | 31.130 | −0.460 | 8.905 | 1.00 | 0.00 | 3A4 |
| ATOM | 2079 | OE1 | GLU | 307 | 30.600 | 0.159 | 7.942 | 1.00 | 0.00 | 3A4 |
| ATOM | 2080 | OE2 | GLU | 307 | 31.670 | −1.592 | 8.778 | 1.00 | 0.00 | 3A4 |
| ATOM | 2081 | C | GLU | 307 | 30.630 | 3.933 | 9.563 | 1.00 | 0.00 | 3A4 |
| ATOM | 2082 | O | GLU | 307 | 29.605 | 3.926 | 8.924 | 1.00 | 0.00 | 3A4 |
| ATOM | 2083 | N | THR | 308 | 31.140 | 5.089 | 10.026 | 1.00 | 0.00 | 3A4 |
| ATOM | 2084 | CA | THR | 308 | 30.556 | 6.394 | 9.788 | 1.00 | 0.00 | 3A4 |
| ATOM | 2085 | CB | THR | 308 | 30.866 | 7.363 | 10.932 | 1.00 | 0.00 | 3A4 |
| ATOM | 2086 | OG1 | THR | 308 | 32.255 | 7.423 | 11.254 | 1.00 | 0.00 | 3A4 |
| ATOM | 2087 | CG2 | THR | 308 | 30.088 | 6.935 | 12.191 | 1.00 | 0.00 | 3A4 |
| ATOM | 2088 | C | THR | 308 | 31.006 | 6.957 | 8.463 | 1.00 | 0.00 | 3A4 |
| ATOM | 2089 | O | THR | 308 | 30.202 | 7.360 | 7.632 | 1.00 | 0.00 | 3A4 |
| ATOM | 2090 | N | THR | 309 | 32.332 | 6.929 | 8.226 | 1.00 | 0.00 | 3A4 |
| ATOM | 2091 | CA | THR | 309 | 32.979 | 7.436 | 7.038 | 1.00 | 0.00 | 3A4 |
| ATOM | 2092 | CB | THR | 309 | 34.479 | 7.445 | 7.204 | 1.00 | 0.00 | 3A4 |
| ATOM | 2093 | OG1 | THR | 309 | 34.937 | 6.213 | 7.743 | 1.00 | 0.00 | 3A4 |
| ATOM | 2094 | CG2 | THR | 309 | 34.867 | 8.555 | 8.200 | 1.00 | 0.00 | 3A4 |
| ATOM | 2095 | C | THR | 309 | 32.600 | 6.684 | 5.779 | 1.00 | 0.00 | 3A4 |
| ATOM | 2096 | O | THR | 309 | 32.349 | 7.278 | 4.741 | 1.00 | 0.00 | 3A4 |
| ATOM | 2097 | N | SER | 310 | 32.484 | 5.339 | 5.875 | 1.00 | 0.00 | 3A4 |
| ATOM | 2098 | CA | SER | 310 | 32.052 | 4.458 | 4.809 | 1.00 | 0.00 | 3A4 |
| ATOM | 2099 | CB | SER | 310 | 31.996 | 2.990 | 5.310 | 1.00 | 0.00 | 3A4 |
| ATOM | 2100 | OG | SER | 310 | 33.306 | 2.563 | 5.646 | 1.00 | 0.00 | 3A4 |
| ATOM | 2101 | C | SER | 310 | 30.666 | 4.763 | 4.322 | 1.00 | 0.00 | 3A4 |
| ATOM | 2102 | O | SER | 310 | 30.413 | 4.886 | 3.131 | 1.00 | 0.00 | 3A4 |
| ATOM | 2103 | N | SER | 311 | 29.728 | 4.960 | 5.261 | 1.00 | 0.00 | 3A4 |
| ATOM | 2104 | CA | SER | 311 | 28.351 | 5.245 | 4.950 | 1.00 | 0.00 | 3A4 |
| ATOM | 2105 | CB | SER | 311 | 27.509 | 5.056 | 6.222 | 1.00 | 0.00 | 3A4 |
| ATOM | 2106 | OG | SER | 311 | 27.701 | 3.739 | 6.719 | 1.00 | 0.00 | 3A4 |
| ATOM | 2107 | C | SER | 311 | 28.153 | 6.627 | 4.389 | 1.00 | 0.00 | 3A4 |
| ATOM | 2108 | O | SER | 311 | 27.316 | 6.833 | 3.523 | 1.00 | 0.00 | 3A4 |
| ATOM | 2109 | N | VAL | 312 | 28.984 | 7.609 | 4.813 | 1.00 | 0.00 | 3A4 |
| ATOM | 2110 | CA | VAL | 312 | 28.964 | 8.970 | 4.310 | 1.00 | 0.00 | 3A4 |
| ATOM | 2111 | CB | VAL | 312 | 29.781 | 9.902 | 5.173 | 1.00 | 0.00 | 3A4 |
| ATOM | 2112 | CG1 | VAL | 312 | 29.980 | 11.315 | 4.570 | 1.00 | 0.00 | 3A4 |
| ATOM | 2113 | CG2 | VAL | 312 | 28.894 | 10.077 | 6.433 | 1.00 | 0.00 | 3A4 |
| ATOM | 2114 | C | VAL | 312 | 29.408 | 9.072 | 2.884 | 1.00 | 0.00 | 3A4 |
| ATOM | 2115 | O | VAL | 312 | 28.751 | 9.739 | 2.103 | 1.00 | 0.00 | 3A4 |
| ATOM | 2116 | N | LEU | 313 | 30.463 | 8.328 | 2.495 | 1.00 | 0.00 | 3A4 |
| ATOM | 2117 | CA | LEU | 313 | 30.996 | 8.277 | 1.150 | 1.00 | 0.00 | 3A4 |
| ATOM | 2118 | CB | LEU | 313 | 32.294 | 7.466 | 1.085 | 1.00 | 0.00 | 3A4 |
| ATOM | 2119 | CG | LEU | 313 | 33.512 | 8.219 | 1.649 | 1.00 | 0.00 | 3A4 |
| ATOM | 2120 | CD1 | LEU | 313 | 34.492 | 8.511 | 0.499 | 1.00 | 0.00 | 3A4 |
| ATOM | 2121 | CD2 | LEU | 313 | 33.322 | 9.485 | 2.496 | 1.00 | 0.00 | 3A4 |
| ATOM | 2122 | C | LEU | 313 | 30.017 | 7.696 | 0.167 | 1.00 | 0.00 | 3A4 |
| ATOM | 2123 | O | LEU | 313 | 29.808 | 8.229 | −0.917 | 1.00 | 0.00 | 3A4 |
| ATOM | 2124 | N | SER | 314 | 29.309 | 6.624 | 0.579 | 1.00 | 0.00 | 3A4 |
| ATOM | 2125 | CA | SER | 314 | 28.305 | 5.981 | −0.243 | 1.00 | 0.00 | 3A4 |
| ATOM | 2126 | CB | SER | 314 | 27.992 | 4.570 | 0.299 | 1.00 | 0.00 | 3A4 |
| ATOM | 2127 | OG | SER | 314 | 29.195 | 3.821 | 0.397 | 1.00 | 0.00 | 3A4 |
| ATOM | 2128 | C | SER | 314 | 27.070 | 6.845 | −0.420 | 1.00 | 0.00 | 3A4 |
| ATOM | 2129 | O | SER | 314 | 26.505 | 6.909 | −1.507 | 1.00 | 0.00 | 3A4 |
| ATOM | 2130 | N | PHE | 315 | 26.702 | 7.636 | 0.625 | 1.00 | 0.00 | 3A4 |
| ATOM | 2131 | CA | PHE | 315 | 25.595 | 8.572 | 0.583 | 1.00 | 0.00 | 3A4 |
| ATOM | 2132 | CB | PHE | 315 | 25.181 | 9.076 | 2.015 | 1.00 | 0.00 | 3A4 |
| ATOM | 2133 | CG | PHE | 315 | 23.897 | 8.407 | 2.456 | 1.00 | 0.00 | 3A4 |
| ATOM | 2134 | CD1 | PHE | 315 | 23.856 | 7.678 | 3.662 | 1.00 | 0.00 | 3A4 |
| ATOM | 2135 | CD2 | PHE | 315 | 22.732 | 8.451 | 1.660 | 1.00 | 0.00 | 3A4 |
| ATOM | 2136 | CE1 | PHE | 315 | 22.683 | 7.024 | 4.067 | 1.00 | 0.00 | 3A4 |
| ATOM | 2137 | CE2 | PHE | 315 | 21.568 | 7.770 | 2.047 | 1.00 | 0.00 | 3A4 |
| ATOM | 2138 | CZ | PHE | 315 | 21.542 | 7.061 | 3.254 | 1.00 | 0.00 | 3A4 |
| ATOM | 2139 | C | PHE | 315 | 25.938 | 9.760 | −0.291 | 1.00 | 0.00 | 3A4 |
| ATOM | 2140 | O | PHE | 315 | 25.075 | 10.250 | −1.011 | 1.00 | 0.00 | 3A4 |
| ATOM | 2141 | N | ILE | 316 | 27.230 | 10.200 | −0.328 | 1.00 | 0.00 | 3A4 |
| ATOM | 2142 | CA | ILE | 316 | 27.669 | 11.286 | −1.202 | 1.00 | 0.00 | 3A4 |
| ATOM | 2143 | CB | ILE | 316 | 29.119 | 11.864 | −1.060 | 1.00 | 0.00 | 3A4 |
| ATOM | 2144 | CG2 | ILE | 316 | 29.128 | 13.291 | −1.688 | 1.00 | 0.00 | 3A4 |
| ATOM | 2145 | CG1 | ILE | 316 | 29.687 | 11.833 | 0.371 | 1.00 | 0.00 | 3A4 |
| ATOM | 2146 | CD | ILE | 316 | 30.950 | 12.629 | 0.687 | 1.00 | 0.00 | 3A4 |
| ATOM | 2147 | C | ILE | 316 | 27.559 | 10.926 | −2.666 | 1.00 | 0.00 | 3A4 |
| ATOM | 2148 | O | ILE | 316 | 27.111 | 11.726 | −3.476 | 1.00 | 0.00 | 3A4 |
| ATOM | 2149 | N | MET | 317 | 27.919 | 9.671 | −3.009 | 1.00 | 0.00 | 3A4 |
| ATOM | 2150 | CA | MET | 317 | 27.868 | 9.137 | −4.351 | 1.00 | 0.00 | 3A4 |
| ATOM | 2151 | CB | MET | 317 | 28.640 | 7.803 | −4.441 | 1.00 | 0.00 | 3A4 |
| ATOM | 2152 | CG | MET | 317 | 30.148 | 8.007 | −4.191 | 1.00 | 0.00 | 3A4 |
| ATOM | 2153 | SD | MET | 317 | 31.027 | 6.548 | −3.550 | 1.00 | 0.00 | 3A4 |
| ATOM | 2154 | CE | MET | 317 | 32.475 | 7.489 | −2.987 | 1.00 | 0.00 | 3A4 |
| ATOM | 2155 | C | MET | 317 | 26.449 | 8.978 | −4.853 | 1.00 | 0.00 | 3A4 |
| ATOM | 2156 | O | MET | 317 | 26.139 | 9.327 | −5.986 | 1.00 | 0.00 | 3A4 |
| ATOM | 2157 | N | TYR | 318 | 25.516 | 8.539 | −3.981 | 1.00 | 0.00 | 3A4 |
| ATOM | 2158 | CA | TYR | 318 | 24.101 | 8.458 | −4.293 | 1.00 | 0.00 | 3A4 |
| ATOM | 2159 | CB | TYR | 318 | 23.333 | 7.794 | −3.134 | 1.00 | 0.00 | 3A4 |
| ATOM | 2160 | CG | TYR | 318 | 21.822 | 7.683 | −3.312 | 1.00 | 0.00 | 3A4 |
| ATOM | 2161 | CD1 | TYR | 318 | 21.269 | 7.039 | −4.436 | 1.00 | 0.00 | 3A4 |
| ATOM | 2162 | CD2 | TYR | 318 | 20.950 | 8.269 | 2.372 | 1.00 | 0.00 | 3A4 |
| ATOM | 2163 | CE1 | TYR | 318 | 19.881 | 6.987 | −4.624 | 1.00 | 0.00 | 3A4 |
| ATOM | 2164 | CE2 | TYR | 318 | 19.560 | 8.209 | −2.548 | 1.00 | 0.00 | 3A4 |
| ATOM | 2165 | CZ | TYR | 318 | 19.024 | 7.566 | −3.675 | 1.00 | 0.00 | 3A4 |
| ATOM | 2166 | OH | TYR | 318 | 17.625 | 7.498 | −3.855 | 1.00 | 0.00 | 3A4 |
| ATOM | 2167 | C | TYR | 318 | 23.501 | 9.814 | −4.592 | 1.00 | 0.00 | 3A4 |
| ATOM | 2168 | O | TYR | 318 | 22.759 | 9.967 | −5.553 | 1.00 | 0.00 | 3A4 |
| ATOM | 2169 | N | GLU | 319 | 23.864 | 10.847 | −3.796 | 1.00 | 0.00 | 3A4 |
| ATOM | 2170 | CA | GLU | 319 | 23.389 | 12.203 | −3.970 | 1.00 | 0.00 | 3A4 |
| ATOM | 2171 | CB | GLU | 319 | 23.703 | 13.127 | −2.775 | 1.00 | 0.00 | 3A4 |
| ATOM | 2172 | CG | GLU | 319 | 22.849 | 12.760 | −1.559 | 1.00 | 0.00 | 3A4 |
| ATOM | 2173 | CD | GLU | 319 | 23.038 | 13.838 | −0.506 | 1.00 | 0.00 | 3A4 |
| ATOM | 2174 | OE1 | GLU | 319 | 24.202 | 14.049 | −0.085 | 1.00 | 0.00 | 3A4 |
| ATOM | 2175 | OE2 | GLU | 319 | 22.027 | 14.476 | −0.119 | 1.00 | 0.00 | 3A4 |
| ATOM | 2176 | C | GLU | 319 | 23.894 | 12.847 | −5.231 | 1.00 | 0.00 | 3A4 |
| ATOM | 2177 | O | GLU | 319 | 23.132 | 13.474 | −5.951 | 1.00 | 0.00 | 3A4 |
| ATOM | 2178 | N | LEU | 320 | 25.170 | 12.641 | −5.595 | 1.00 | 0.00 | 3A4 |
| ATOM | 2179 | CA | LEU | 320 | 25.713 | 13.161 | −6.828 | 1.00 | 0.00 | 3A4 |
| ATOM | 2180 | CB | LEU | 320 | 27.239 | 13.059 | −6.841 | 1.00 | 0.00 | 3A4 |
| ATOM | 2181 | CG | LEU | 320 | 27.880 | 14.027 | −5.843 | 1.00 | 0.00 | 3A4 |
| ATOM | 2182 | CD1 | LEU | 320 | 29.380 | 13.757 | −5.709 | 1.00 | 0.00 | 3A4 |
| ATOM | 2183 | CD2 | LEU | 320 | 27.611 | 15.489 | −6.192 | 1.00 | 0.00 | 3A4 |
| ATOM | 2184 | C | LEU | 320 | 25.160 | 12.475 | −8.048 | 1.00 | 0.00 | 3A4 |
| ATOM | 2185 | O | LEU | 320 | 24.977 | 13.105 | −9.083 | 1.00 | 0.00 | 3A4 |
| ATOM | 2186 | N | ALA | 321 | 24.818 | 11.177 | −7.933 | 1.00 | 0.00 | 3A4 |
| ATOM | 2187 | CA | ALA | 321 | 24.215 | 10.397 | −8.990 | 1.00 | 0.00 | 3A4 |
| ATOM | 2188 | CB | ALA | 321 | 24.217 | 8.897 | −8.645 | 1.00 | 0.00 | 3A4 |
| ATOM | 2189 | C | ALA | 321 | 22.802 | 10.861 | −9.259 | 1.00 | 0.00 | 3A4 |
| ATOM | 2190 | O | ALA | 321 | 22.338 | 10.884 | −10.397 | 1.00 | 0.00 | 3A4 |
| ATOM | 2191 | N | THR | 322 | 22.103 | 11.305 | −8.194 | 1.00 | 0.00 | 3A4 |
| ATOM | 2192 | CA | THR | 322 | 20.743 | 11.780 | −8.258 | 1.00 | 0.00 | 3A4 |
| ATOM | 2193 | CB | THR | 322 | 19.981 | 11.519 | −6.972 | 1.00 | 0.00 | 3A4 |
| ATOM | 2194 | OG1 | THR | 322 | 20.610 | 11.999 | −5.794 | 1.00 | 0.00 | 3A4 |
| ATOM | 2195 | CG2 | THR | 322 | 19.745 | 9.994 | −6.849 | 1.00 | 0.00 | 3A4 |
| ATOM | 2196 | C | THR | 322 | 20.627 | 13.242 | −6.642 | 1.00 | 0.00 | 3A4 |
| ATOM | 2197 | O | THR | 322 | 19.555 | 13.704 | −9.018 | 1.00 | 0.00 | 3A4 |
| ATOM | 2198 | N | HIS | 323 | 21.731 | 14.006 | −8.558 | 1.00 | 0.00 | 3A4 |
| ATOM | 2199 | CA | HIS | 323 | 21.740 | 15.416 | −8.856 | 1.00 | 0.00 | 3A4 |
| ATOM | 2200 | ND1 | HIS | 323 | 20.544 | 15.212 | −5.650 | 1.00 | 0.00 | 3A4 |
| ATOM | 2201 | CG | HIS | 323 | 20.676 | 16.147 | −6.653 | 1.00 | 0.00 | 3A4 |
| ATOM | 2202 | CB | HIS | 323 | 21.859 | 16.259 | −7.581 | 1.00 | 0.00 | 3A4 |
| ATOM | 2203 | NE2 | HIS | 323 | 18.715 | 16.470 | −5.582 | 1.00 | 0.00 | 3A4 |
| ATOM | 2204 | CD2 | HIS | 323 | 19.549 | 16.908 | −6.595 | 1.00 | 0.00 | 3A4 |
| ATOM | 2205 | CE1 | HIS | 323 | 19.356 | 15.449 | −5.043 | 1.00 | 0.00 | 3A4 |
| ATOM | 2206 | C | HIS | 323 | 22.892 | 15.711 | −9.775 | 1.00 | 0.00 | 3A4 |
| ATOM | 2207 | O | HIS | 323 | 23.883 | 16.309 | −9.352 | 1.00 | 0.00 | 3A4 |
| ATOM | 2208 | N | PRO | 324 | 22.833 | 15.378 | −11.076 | 1.00 | 0.00 | 3A4 |
| ATOM | 2209 | CA | PRO | 324 | 23.958 | 15.508 | −12.002 | 1.00 | 0.00 | 3A4 |
| ATOM | 2210 | CD | PRO | 324 | 21.602 | 14.983 | −11.765 | 1.00 | 0.00 | 3A4 |
| ATOM | 2211 | CB | PRO | 324 | 23.420 | 14.910 | −13.300 | 1.00 | 0.00 | 3A4 |
| ATOM | 2212 | CG | PRO | 324 | 21.905 | 15.129 | −13.255 | 1.00 | 0.00 | 3A4 |
| ATOM | 2213 | C | PRO | 324 | 24.415 | 16.938 | −12.213 | 1.00 | 0.00 | 3A4 |
| ATOM | 2214 | O | PRO | 324 | 25.575 | 17.158 | −12.534 | 1.00 | 0.00 | 3A4 |
| ATOM | 2215 | N | ASP | 325 | 23.561 | 17.950 | −11.949 | 1.00 | 0.00 | 3A4 |
| ATOM | 2216 | CA | ASP | 325 | 23.917 | 19.351 | −12.002 | 1.00 | 0.00 | 3A4 |
| ATOM | 2217 | CB | ASP | 325 | 22.668 | 20.245 | −11.821 | 1.00 | 0.00 | 3A4 |
| ATOM | 2218 | CG | ASP | 325 | 21.653 | 19.933 | −12.931 | 1.00 | 0.00 | 3A4 |
| ATOM | 2219 | OD1 | ASP | 325 | 21.991 | 20.159 | −14.125 | 1.00 | 0.00 | 3A4 |
| ATOM | 2220 | OD2 | ASP | 325 | 20.531 | 19.463 | −12.600 | 1.00 | 0.00 | 3A4 |
| ATOM | 2221 | C | ASP | 325 | 24.927 | 19.731 | −10.944 | 1.00 | 0.00 | 3A4 |
| ATOM | 2222 | O | ASP | 325 | 25.817 | 20.547 | −11.169 | 1.00 | 0.00 | 3A4 |
| ATOM | 2223 | N | VAL | 326 | 24.835 | 19.071 | −9.767 | 1.00 | 0.00 | 3A4 |
| ATOM | 2224 | CA | VAL | 326 | 25.742 | 19.249 | −8.647 | 1.00 | 0.00 | 3A4 |
| ATOM | 2225 | CB | VAL | 326 | 25.198 | 18.672 | −7.341 | 1.00 | 0.00 | 3A4 |
| ATOM | 2226 | CG1 | VAL | 326 | 26.201 | 18.872 | −6.173 | 1.00 | 0.00 | 3A4 |
| ATOM | 2227 | CG2 | VAL | 326 | 23.874 | 19.402 | −7.034 | 1.00 | 0.00 | 3A4 |
| ATOM | 2228 | C | VAL | 326 | 27.083 | 18.648 | −8.960 | 1.00 | 0.00 | 3A4 |
| ATOM | 2229 | O | VAL | 326 | 28.106 | 19.288 | −8.782 | 1.00 | 0.00 | 3A4 |
| ATOM | 2230 | N | GLN | 327 | 27.089 | 17.427 | −9.528 | 1.00 | 0.00 | 3A4 |
| ATOM | 2231 | CA | GLN | 327 | 28.281 | 16.718 | −9.931 | 1.00 | 0.00 | 3A4 |
| ATOM | 2232 | CB | GLN | 327 | 27.899 | 15.340 | −10.487 | 1.00 | 0.00 | 3A4 |
| ATOM | 2233 | CG | GLN | 327 | 29.062 | 14.350 | −10.683 | 1.00 | 0.00 | 3A4 |
| ATOM | 2234 | CD | GLN | 327 | 28.538 | 12.991 | −11.166 | 1.00 | 0.00 | 3A4 |
| ATOM | 2235 | OE1 | GLN | 327 | 27.336 | 12.728 | −11.186 | 1.00 | 0.00 | 3A4 |
| ATOM | 2236 | NE2 | GLN | 327 | 29.475 | 12.091 | −11.572 | 1.00 | 0.00 | 3A4 |
| ATOM | 2237 | C | GLN | 327 | 29.088 | 17.459 | −10.960 | 1.00 | 0.00 | 3A4 |
| ATOM | 2238 | O | GLN | 327 | 30.301 | 17.551 | −10.855 | 1.00 | 0.00 | 3A4 |
| ATOM | 2239 | N | GLN | 328 | 28.408 | 18.092 | −11.941 | 1.00 | 0.00 | 3A4 |
| ATOM | 2240 | CA | GLN | 328 | 29.023 | 18.879 | −12.995 | 1.00 | 0.00 | 3A4 |
| ATOM | 2241 | CB | GLN | 328 | 27.977 | 19.321 | −14.041 | 1.00 | 0.00 | 3A4 |
| ATOM | 2242 | CG | GLN | 328 | 27.544 | 18.163 | −14.957 | 1.00 | 0.00 | 3A4 |
| ATOM | 2243 | CD | GLN | 328 | 26.423 | 18.643 | −15.885 | 1.00 | 0.00 | 3A4 |
| ATOM | 2244 | OE1 | GLN | 328 | 26.675 | 19.426 | −16.800 | 1.00 | 0.00 | 3A4 |
| ATOM | 2245 | NE2 | GLN | 328 | 25.164 | 18.178 | −15.654 | 1.00 | 0.00 | 3A4 |
| ATOM | 2246 | C | GLN | 328 | 29.711 | 20.106 | −12.470 | 1.00 | 0.00 | 3A4 |
| ATOM | 2247 | O | GLN | 328 | 30.827 | 20.429 | −12.855 | 1.00 | 0.00 | 3A4 |
| ATOM | 2248 | N | LYS | 329 | 29.068 | 20.782 | −11.501 | 1.00 | 0.00 | 3A4 |
| ATOM | 2249 | CA | LYS | 329 | 29.597 | 21.931 | −10.807 | 1.00 | 0.00 | 3A4 |
| ATOM | 2250 | CB | LYS | 329 | 28.515 | 22.465 | −9.850 | 1.00 | 0.00 | 3A4 |
| ATOM | 2251 | CG | LYS | 329 | 28.752 | 23.857 | −9.250 | 1.00 | 0.00 | 3A4 |
| ATOM | 2252 | CD | LYS | 329 | 27.533 | 24.347 | −8.460 | 1.00 | 0.00 | 3A4 |
| ATOM | 2253 | CE | LYS | 329 | 27.679 | 25.785 | −7.953 | 1.00 | 0.00 | 3A4 |
| ATOM | 2254 | NZ | LYS | 329 | 26.477 | 26.210 | −7.202 | 1.00 | 0.00 | 3A4 |
| ATOM | 2255 | C | LYS | 329 | 30.884 | 21.621 | −10.052 | 1.00 | 0.00 | 3A4 |
| ATOM | 2256 | O | LYS | 329 | 31.860 | 22.366 | −10.101 | 1.00 | 0.00 | 3A4 |
| ATOM | 2257 | N | LEU | 330 | 30.952 | 20.423 | −9.426 | 1.00 | 0.00 | 3A4 |
| ATOM | 2258 | CA | LEU | 330 | 32.100 | 19.968 | −8.670 | 1.00 | 0.00 | 3A4 |
| ATOM | 2259 | CB | LEU | 330 | 31.814 | 18.742 | −7.797 | 1.00 | 0.00 | 3A4 |
| ATOM | 2260 | CG | LEU | 330 | 30.502 | 18.756 | −7.038 | 1.00 | 0.00 | 3A4 |
| ATOM | 2261 | CD1 | LEU | 330 | 30.541 | 17.732 | −5.920 | 1.00 | 0.00 | 3A4 |
| ATOM | 2262 | CD2 | LEU | 330 | 29.986 | 20.145 | −6.584 | 1.00 | 0.00 | 3A4 |
| ATOM | 2263 | C | LEU | 330 | 33.250 | 19.598 | −9.579 | 1.00 | 0.00 | 3A4 |
| ATOM | 2264 | O | LEU | 330 | 34.410 | 19.864 | −9.296 | 1.00 | 0.00 | 3A4 |
| ATOM | 2265 | N | GLN | 331 | 32.931 | 18.993 | −10.741 | 1.00 | 0.00 | 3A4 |
| ATOM | 2266 | CA | GLN | 331 | 33.881 | 18.627 | −11.762 | 1.00 | 0.00 | 3A4 |
| ATOM | 2267 | CB | GLN | 331 | 33.229 | 17.787 | −12.880 | 1.00 | 0.00 | 3A4 |
| ATOM | 2268 | CG | GLN | 331 | 32.942 | 16.354 | −12.389 | 1.00 | 0.00 | 3A4 |
| ATOM | 2269 | CD | GLN | 331 | 32.147 | 15.568 | −13.437 | 1.00 | 0.00 | 3A4 |
| ATOM | 2270 | OE1 | GLN | 331 | 31.063 | 15.988 | −13.839 | 1.00 | 0.00 | 3A4 |
| ATOM | 2271 | NE2 | GLN | 331 | 32.677 | 14.396 | −13.885 | 1.00 | 0.00 | 3A4 |
| ATOM | 2272 | C | GLN | 331 | 34.500 | 19.817 | −12.447 | 1.00 | 0.00 | 3A4 |
| ATOM | 2273 | O | GLN | 331 | 35.682 | 19.821 | −12.771 | 1.00 | 0.00 | 3A4 |
| ATOM | 2274 | N | GLU | 332 | 33.723 | 20.913 | −12.594 | 1.00 | 0.00 | 3A4 |
| ATOM | 2275 | CA | GLU | 332 | 34.188 | 22.185 | −13.099 | 1.00 | 0.00 | 3A4 |
| ATOM | 2276 | CB | GLU | 332 | 33.019 | 23.173 | −13.413 | 1.00 | 0.00 | 3A4 |
| ATOM | 2277 | CG | GLU | 332 | 32.212 | 22.808 | −14.675 | 1.00 | 0.00 | 3A4 |
| ATOM | 2278 | CD | GLU | 332 | 33.080 | 22.951 | −15.931 | 1.00 | 0.00 | 3A4 |
| ATOM | 2279 | OE1 | GLU | 332 | 33.533 | 24.092 | −16.217 | 1.00 | 0.00 | 3A4 |
| ATOM | 2280 | OE2 | GLU | 332 | 33.301 | 21.919 | −16.622 | 1.00 | 0.00 | 3A4 |
| ATOM | 2281 | C | GLU | 332 | 35.190 | 22.869 | −12.182 | 1.00 | 0.00 | 3A4 |
| ATOM | 2282 | O | GLU | 332 | 35.834 | 23.799 | −12.629 | 1.00 | 0.00 | 3A4 |
| ATOM | 2283 | N | GLU | 333 | 35.484 | 22.350 | −10.947 | 1.00 | 0.00 | 3A4 |
| ATOM | 2284 | CA | GLU | 333 | 36.662 | 22.661 | −10.132 | 1.00 | 0.00 | 3A4 |
| ATOM | 2285 | CB | GLU | 333 | 36.449 | 22.284 | −8.639 | 1.00 | 0.00 | 3A4 |
| ATOM | 2286 | CG | GLU | 333 | 35.228 | 22.936 | −7.985 | 1.00 | 0.00 | 3A4 |
| ATOM | 2287 | CD | GLU | 333 | 35.046 | 22.273 | −6.620 | 1.00 | 0.00 | 3A4 |
| ATOM | 2288 | OE1 | GLU | 333 | 35.918 | 22.455 | −5.730 | 1.00 | 0.00 | 3A4 |
| ATOM | 2289 | OE2 | GLU | 333 | 34.038 | 21.540 | −6.462 | 1.00 | 0.00 | 3A4 |
| ATOM | 2290 | C | GLU | 333 | 37.923 | 21.912 | −10.581 | 1.00 | 0.00 | 3A4 |
| ATOM | 2291 | O | GLU | 333 | 38.425 | 21.038 | −9.874 | 1.00 | 0.00 | 3A4 |
| ATOM | 2292 | N | ILE | 334 | 38.469 | 22.262 | −11.773 | 1.00 | 0.00 | 3A4 |
| ATOM | 2293 | CA | ILE | 334 | 39.710 | 21.726 | −12.300 | 1.00 | 0.00 | 3A4 |
| ATOM | 2294 | CB | ILE | 334 | 39.559 | 20.353 | −12.973 | 1.00 | 0.00 | 3A4 |
| ATOM | 2295 | CG2 | ILE | 334 | 38.582 | 20.368 | −14.182 | 1.00 | 0.00 | 3A4 |
| ATOM | 2296 | CG1 | ILE | 334 | 40.932 | 19.687 | −13.257 | 1.00 | 0.00 | 3A4 |
| ATOM | 2297 | CD | ILE | 334 | 40.838 | 18.213 | −13.660 | 1.00 | 0.00 | 3A4 |
| ATOM | 2298 | C | ILE | 334 | 40.231 | 22.836 | −13.189 | 1.00 | 0.00 | 3A4 |
| ATOM | 2299 | O | ILE | 334 | 40.570 | 22.658 | −14.358 | 1.00 | 0.00 | 3A4 |
| ATOM | 2300 | N | ASP | 335 | 40.294 | 24.063 | −12.600 | 1.00 | 0.00 | 3A4 |
| ATOM | 2301 | CA | ASP | 335 | 40.593 | 25.320 | −13.260 | 1.00 | 0.00 | 3A4 |
| ATOM | 2302 | CB | ASP | 335 | 39.726 | 26.490 | −12.699 | 1.00 | 0.00 | 3A4 |
| ATOM | 2303 | CG | ASP | 335 | 38.240 | 26.203 | −12.943 | 1.00 | 0.00 | 3A4 |
| ATOM | 2304 | OD1 | ASP | 335 | 37.854 | 26.015 | −14.129 | 1.00 | 0.00 | 3A4 |
| ATOM | 2305 | OD2 | ASP | 335 | 37.469 | 26.175 | −11.947 | 1.00 | 0.00 | 3A4 |
| ATOM | 2306 | C | ASP | 335 | 42.054 | 25.655 | −13.079 | 1.00 | 0.00 | 3A4 |
| ATOM | 2307 | O | ASP | 335 | 42.537 | 25.781 | −11.956 | 1.00 | 0.00 | 3A4 |
| ATOM | 2308 | N | ALA | 336 | 42.772 | 25.816 | −14.223 | 1.00 | 0.00 | 3A4 |
| ATOM | 2309 | CA | ALA | 336 | 44.168 | 26.192 | −14.316 | 1.00 | 0.00 | 3A4 |
| ATOM | 2310 | CB | ALA | 336 | 45.027 | 25.116 | −15.028 | 1.00 | 0.00 | 3A4 |
| ATOM | 2311 | C | ALA | 336 | 44.207 | 27.500 | −15.065 | 1.00 | 0.00 | 3A4 |
| ATOM | 2312 | O | ALA | 336 | 44.428 | 27.544 | −16.275 | 1.00 | 0.00 | 3A4 |
| ATOM | 2313 | N | VAL | 337 | 43.963 | 28.609 | −14.313 | 1.00 | 0.00 | 3A4 |
| ATOM | 2314 | CA | VAL | 337 | 43.815 | 29.978 | −14.773 | 1.00 | 0.00 | 3A4 |
| ATOM | 2315 | CB | VAL | 337 | 42.641 | 30.686 | −14.085 | 1.00 | 0.00 | 3A4 |
| ATOM | 2316 | CG1 | VAL | 337 | 42.436 | 32.130 | −14.612 | 1.00 | 0.00 | 3A4 |
| ATOM | 2311 | CG2 | VAL | 337 | 41.367 | 29.847 | −14.326 | 1.00 | 0.00 | 3A4 |
| ATOM | 2318 | C | VAL | 337 | 45.115 | 30.696 | −14.492 | 1.00 | 0.00 | 3A4 |
| ATOM | 2319 | O | VAL | 337 | 45.661 | 30.605 | −13.394 | 1.00 | 0.00 | 3A4 |
| ATOM | 2320 | N | LEU | 338 | 45.621 | 31.439 | −15.517 | 1.00 | 0.00 | 3A4 |
| ATOM | 2321 | CA | LEU | 338 | 46.821 | 32.257 | −15.500 | 1.00 | 0.00 | 3A4 |
| ATOM | 2322 | CB | LEU | 338 | 47.678 | 32.088 | −16.799 | 1.00 | 0.00 | 3A4 |
| ATOM | 2323 | CG | LEU | 338 | 48.507 | 30.778 | −16.897 | 1.00 | 0.00 | 3A4 |
| ATOM | 2324 | CD1 | LEU | 338 | 47.680 | 29.494 | −17.124 | 1.00 | 0.00 | 3A4 |
| ATOM | 2325 | CD2 | LEU | 338 | 49.592 | 30.909 | −17.984 | 1.00 | 0.00 | 3A4 |
| ATOM | 2326 | C | LEU | 338 | 46.355 | 33.705 | −15.361 | 1.00 | 0.00 | 3A4 |
| ATOM | 2327 | O | LEU | 338 | 45.265 | 34.009 | −15.844 | 1.00 | 0.00 | 3A4 |
| ATOM | 2328 | N | PRO | 339 | 47.103 | 34.638 | −14.719 | 1.00 | 0.00 | 3A4 |
| ATOM | 2329 | CA | PRO | 339 | 46.612 | 35.949 | −14.286 | 1.00 | 0.00 | 3A4 |
| ATOM | 2330 | CD | PRO | 339 | 48.443 | 34.363 | −14.196 | 1.00 | 0.00 | 3A4 |
| ATOM | 2331 | CB | PRO | 339 | 47.610 | 36.376 | −13.189 | 1.00 | 0.00 | 3A4 |
| ATOM | 2332 | CG | PRO | 339 | 48.922 | 35.669 | −13.552 | 1.00 | 0.00 | 3A4 |
| ATOM | 2333 | C | PRO | 339 | 46.561 | 36.955 | −15.437 | 1.00 | 0.00 | 3A4 |
| ATOM | 2334 | O | PRO | 339 | 47.465 | 36.980 | −16.272 | 1.00 | 0.00 | 3A4 |
| ATOM | 2335 | N | ASN | 340 | 45.491 | 37.790 | −15.465 | 1.00 | 0.00 | 3A4 |
| ATOM | 2336 | CA | ASN | 340 | 45.246 | 38.810 | −16.464 | 1.00 | 0.00 | 3A4 |
| ATOM | 2337 | CB | ASN | 340 | 44.189 | 38.343 | −17.523 | 1.00 | 0.00 | 3A4 |
| ATOM | 2338 | CG | ASN | 340 | 44.036 | 39.320 | −18.704 | 1.00 | 0.00 | 3A4 |
| ATOM | 2339 | OD1 | ASN | 340 | 42.986 | 39.942 | −18.858 | 1.00 | 0.00 | 3A4 |
| ATOM | 2340 | ND2 | ASN | 340 | 45.094 | 39.457 | −19.550 | 1.00 | 0.00 | 3A4 |
| ATOM | 2341 | C | ASN | 340 | 44.785 | 40.012 | −15.672 | 1.00 | 0.00 | 3A4 |
| ATOM | 2342 | O | ASN | 340 | 45.452 | 41.045 | −15.645 | 1.00 | 0.00 | 3A4 |
| ATOM | 2343 | N | LYS | 341 | 43.617 | 39.870 | −14.997 | 1.00 | 0.00 | 3A4 |
| ATOM | 2344 | CA | LYS | 341 | 43.059 | 40.852 | −14.095 | 1.00 | 0.00 | 3A4 |
| ATOM | 2345 | CB | LYS | 341 | 42.250 | 41.970 | −14.822 | 1.00 | 0.00 | 3A4 |
| ATOM | 2346 | CG | LYS | 341 | 41.892 | 43.181 | −13.936 | 1.00 | 0.00 | 3A4 |
| ATOM | 2347 | CD | LYS | 341 | 41.220 | 44.359 | −14.664 | 1.00 | 0.00 | 3A4 |
| ATOM | 2348 | CE | LYS | 341 | 39.708 | 44.212 | −14.924 | 1.00 | 0.00 | 3A4 |
| ATOM | 2349 | NZ | LYS | 341 | 39.409 | 43.231 | −15.994 | 1.00 | 0.00 | 3A4 |
| ATOM | 2350 | C | LYS | 341 | 42.197 | 40.042 | −13.158 | 1.00 | 0.00 | 3A4 |
| ATOM | 2351 | O | LYS | 341 | 40.991 | 40.258 | −13.036 | 1.00 | 0.00 | 3A4 |
| ATOM | 2352 | N | ALA | 342 | 42.834 | 39.053 | −12.482 | 1.00 | 0.00 | 3A4 |
| ATOM | 2353 | CA | ALA | 342 | 42.189 | 38.131 | −11.577 | 1.00 | 0.00 | 3A4 |
| ATOM | 2354 | CB | ALA | 342 | 41.316 | 37.064 | −12.306 | 1.00 | 0.00 | 3A4 |
| ATOM | 2355 | C | ALA | 342 | 43.313 | 37.430 | −10.839 | 1.00 | 0.00 | 3A4 |
| ATOM | 2356 | O | ALA | 342 | 44.413 | 37.344 | −11.386 | 1.00 | 0.00 | 3A4 |
| ATOM | 2357 | N | PRO | 343 | 43.106 | 36.880 | −9.614 | 1.00 | 0.00 | 3A4 |
| ATOM | 2358 | CA | PRO | 343 | 44.072 | 36.050 | −8.891 | 1.00 | 0.00 | 3A4 |
| ATOM | 2359 | CD | PRO | 343 | 41.953 | 37.220 | −8.779 | 1.00 | 0.00 | 3A4 |
| ATOM | 2360 | CB | PRO | 343 | 43.605 | 36.121 | −7.421 | 1.00 | 0.00 | 3A4 |
| ATOM | 2361 | CG | PRO | 343 | 42.097 | 36.392 | −7.496 | 1.00 | 0.00 | 3A4 |
| ATOM | 2362 | C | PRO | 343 | 44.071 | 34.607 | −9.441 | 1.00 | 0.00 | 3A4 |
| ATOM | 2363 | O | PRO | 343 | 42.970 | 34.083 | −9.619 | 1.00 | 0.00 | 3A4 |
| ATOM | 2364 | N | PRO | 344 | 45.209 | 33.930 | −9.727 | 1.00 | 0.00 | 3A4 |
| ATOM | 2365 | CA | PRO | 344 | 45.259 | 32.659 | −10.448 | 1.00 | 0.00 | 3A4 |
| ATOM | 2366 | CD | PRO | 344 | 46.542 | 34.509 | −9.559 | 1.00 | 0.00 | 3A4 |
| ATOM | 2367 | CB | PRO | 344 | 46.678 | 32.636 | −11.047 | 1.00 | 0.00 | 3A4 |
| ATOM | 2368 | CG | PRO | 344 | 47.532 | 33.459 | −10.077 | 1.00 | 0.00 | 3A4 |
| ATOM | 2369 | C | PRO | 344 | 45.002 | 31.473 | −9.520 | 1.00 | 0.00 | 3A4 |
| ATOM | 2370 | O | PRO | 344 | 45.522 | 31.443 | −8.404 | 1.00 | 0.00 | 3A4 |
| ATOM | 2371 | N | THR | 345 | 44.206 | 30.483 | −10.004 | 1.00 | 0.00 | 3A4 |
| ATOM | 2372 | CA | THR | 345 | 43.860 | 29.247 | −9.324 | 1.00 | 0.00 | 3A4 |
| ATOM | 2373 | CB | THR | 345 | 42.343 | 29.124 | −9.107 | 1.00 | 0.00 | 3A4 |
| ATOM | 2374 | OG1 | THR | 345 | 41.981 | 27.994 | −8.315 | 1.00 | 0.00 | 3A4 |
| ATOM | 2375 | CG2 | THR | 345 | 41.532 | 29.140 | −10.430 | 1.00 | 0.00 | 3A4 |
| ATOM | 2376 | C | THR | 345 | 44.457 | 28.142 | −10.173 | 1.00 | 0.00 | 3A4 |
| ATOM | 2377 | O | THR | 345 | 44.489 | 28.242 | −11.397 | 1.00 | 0.00 | 3A4 |
| ATOM | 2378 | N | TYR | 346 | 44.964 | 27.063 | −9.521 | 1.00 | 0.00 | 3A4 |
| ATOM | 2379 | CA | TYR | 346 | 45.601 | 25.934 | −10.173 | 1.00 | 0.00 | 3A4 |
| ATOM | 2380 | CB | TYR | 346 | 47.162 | 25.970 | −10.121 | 1.00 | 0.00 | 3A4 |
| ATOM | 2381 | CG | TYR | 346 | 47.679 | 27.135 | −10.925 | 1.00 | 0.00 | 3A4 |
| ATOM | 2382 | CD1 | TYR | 346 | 48.250 | 28.252 | −10.287 | 1.00 | 0.00 | 3A4 |
| ATOM | 2383 | CD2 | TYR | 346 | 47.581 | 27.136 | −12.331 | 1.00 | 0.00 | 3A4 |
| ATOM | 2384 | CE1 | TYR | 346 | 48.713 | 29.345 | −11.032 | 1.00 | 0.00 | 3A4 |
| ATOM | 2385 | CE2 | TYR | 346 | 48.037 | 28.229 | −13.082 | 1.00 | 0.00 | 3A4 |
| ATOM | 2386 | CZ | TYR | 346 | 48.606 | 29.335 | −12.431 | 1.00 | 0.00 | 3A4 |
| ATOM | 2387 | OH | TYR | 346 | 49.065 | 30.442 | −13.178 | 1.00 | 0.00 | 3A4 |
| ATOM | 2388 | C | TYR | 346 | 45.106 | 24.709 | −9.453 | 1.00 | 0.00 | 3A4 |
| ATOM | 2389 | O | TYR | 346 | 45.423 | 24.491 | −8.285 | 1.00 | 0.00 | 3A4 |
| ATOM | 2390 | N | ASP | 347 | 44.302 | 23.879 | −10.168 | 1.00 | 0.00 | 3A4 |
| ATOM | 2391 | CA | ASP | 347 | 43.715 | 22.647 | −9.683 | 1.00 | 0.00 | 3A4 |
| ATOM | 2392 | CB | ASP | 347 | 42.187 | 22.761 | −9.384 | 1.00 | 0.00 | 3A4 |
| ATOM | 2393 | CG | ASP | 347 | 41.948 | 23.760 | −8.248 | 1.00 | 0.00 | 3A4 |
| ATOM | 2394 | OD1 | ASP | 347 | 41.329 | 24.825 | −8.516 | 1.00 | 0.00 | 3A4 |
| ATOM | 2395 | OD2 | ASP | 347 | 42.386 | 23.474 | −7.102 | 1.00 | 0.00 | 3A4 |
| ATOM | 2396 | C | ASP | 347 | 43.937 | 21.639 | −10.781 | 1.00 | 0.00 | 3A4 |
| ATOM | 2397 | O | ASP | 347 | 43.549 | 21.862 | −11.927 | 1.00 | 0.00 | 3A4 |
| ATOM | 2398 | N | THR | 348 | 44.581 | 20.493 | −10.427 | 1.00 | 0.00 | 3A4 |
| ATOM | 2399 | CA | THR | 348 | 44.908 | 19.388 | −11.314 | 1.00 | 0.00 | 3A4 |
| ATOM | 2400 | CB | THR | 348 | 46.416 | 19.199 | −11.501 | 1.00 | 0.00 | 3A4 |
| ATOM | 2401 | OG1 | THR | 348 | 46.987 | 20.401 | −12.006 | 1.00 | 0.00 | 3A4 |
| ATOM | 2402 | CG2 | THR | 348 | 46.725 | 18.051 | −12.500 | 1.00 | 0.00 | 3A4 |
| ATOM | 2403 | C | THR | 348 | 44.258 | 18.178 | −10.688 | 1.00 | 0.00 | 3A4 |
| ATOM | 2404 | O | THR | 348 | 43.393 | 17.548 | −11.293 | 1.00 | 0.00 | 3A4 |
| ATOM | 2405 | N | VAL | 349 | 44.670 | 17.854 | −9.434 | 1.00 | 0.00 | 3A4 |
| ATOM | 2406 | CA | VAL | 349 | 44.059 | 16.848 | −8.590 | 1.00 | 0.00 | 3A4 |
| ATOM | 2407 | CB | VAL | 349 | 44.630 | 15.437 | −8.788 | 1.00 | 0.00 | 3A4 |
| ATOM | 2408 | CG1 | VAL | 349 | 46.169 | 15.347 | −8.617 | 1.00 | 0.00 | 3A4 |
| ATOM | 2409 | CG2 | VAL | 349 | 43.834 | 14.402 | −7.958 | 1.00 | 0.00 | 3A4 |
| ATOM | 2410 | C | VAL | 349 | 44.175 | 17.435 | −7.201 | 1.00 | 0.00 | 3A4 |
| ATOM | 2411 | O | VAL | 349 | 44.934 | 16.990 | −6.341 | 1.00 | 0.00 | 3A4 |
| ATOM | 2412 | N | LEU | 350 | 43.387 | 18.518 | −6.988 | 1.00 | 0.00 | 3A4 |
| ATOM | 2413 | CA | LEU | 350 | 43.440 | 19.369 | −5.824 | 1.00 | 0.00 | 3A4 |
| ATOM | 2414 | CB | LEU | 350 | 44.479 | 20.520 | −6.024 | 1.00 | 0.00 | 3A4 |
| ATOM | 2415 | CG | LEU | 350 | 44.847 | 21.374 | −4.785 | 1.00 | 0.00 | 3A4 |
| ATOM | 2416 | CD1 | LEU | 350 | 45.497 | 20.539 | −3.662 | 1.00 | 0.00 | 3A4 |
| ATOM | 2417 | CD2 | LEU | 350 | 45.743 | 22.560 | −5.187 | 1.00 | 0.00 | 3A4 |
| ATOM | 2418 | C | LEU | 350 | 42.042 | 19.913 | −5.672 | 1.00 | 0.00 | 3A4 |
| ATOM | 2419 | O | LEU | 350 | 41.270 | 19.945 | −6.631 | 1.00 | 0.00 | 3A4 |
| ATOM | 2420 | N | GLN | 351 | 41.689 | 20.372 | −4.436 | 1.00 | 0.00 | 3A4 |
| ATOM | 2421 | CA | GLN | 351 | 40.404 | 20.942 | −4.059 | 1.00 | 0.00 | 3A4 |
| ATOM | 2422 | CB | GLN | 351 | 40.005 | 20.645 | −2.573 | 1.00 | 0.00 | 3A4 |
| ATOM | 2423 | CG | GLN | 351 | 40.814 | 21.289 | −1.417 | 1.00 | 0.00 | 3A4 |
| ATOM | 2424 | CD | GLN | 351 | 42.269 | 20.816 | −1.386 | 1.00 | 0.00 | 3A4 |
| ATOM | 2425 | OE1 | GLN | 351 | 43.177 | 21.634 | −1.520 | 1.00 | 0.00 | 3A4 |
| ATOM | 2426 | NE2 | GLN | 351 | 42.505 | 19.487 | −1.200 | 1.00 | 0.00 | 3A4 |
| ATOM | 2427 | C | GLN | 351 | 40.390 | 22.432 | −4.305 | 1.00 | 0.00 | 3A4 |
| ATOM | 2428 | O | GLN | 351 | 41.399 | 23.112 | −4.117 | 1.00 | 0.00 | 3A4 |
| ATOM | 2429 | N | MET | 352 | 39.214 | 22.962 | −4.740 | 1.00 | 0.00 | 3A4 |
| ATOM | 2430 | CA | MET | 352 | 38.994 | 24.368 | −5.014 | 1.00 | 0.00 | 3A4 |
| ATOM | 2431 | CB | MET | 352 | 38.436 | 24.632 | −6.438 | 1.00 | 0.00 | 3A4 |
| ATOM | 2432 | CG | MET | 352 | 38.472 | 26.097 | −6.900 | 1.00 | 0.00 | 3A4 |
| ATOM | 2433 | SD | MET | 352 | 37.969 | 26.316 | −8.633 | 1.00 | 0.00 | 3A4 |
| ATOM | 2434 | CE | MET | 352 | 38.075 | 28.129 | −8.608 | 1.00 | 0.00 | 3A4 |
| ATOM | 2435 | C | MET | 352 | 38.113 | 24.911 | −3.914 | 1.00 | 0.00 | 3A4 |
| ATOM | 2436 | O | MET | 352 | 38.636 | 25.287 | −2.866 | 1.00 | 0.00 | 3A4 |
| ATOM | 2437 | N | GLU | 353 | 36.765 | 25.000 | −4.117 | 1.00 | 0.00 | 3A4 |
| ATOM | 2438 | CA | GLU | 353 | 35.914 | 25.749 | −3.211 | 1.00 | 0.00 | 3A4 |
| ATOM | 2439 | CB | GLU | 353 | 35.689 | 27.217 | −3.688 | 1.00 | 0.00 | 3A4 |
| ATOM | 2440 | CG | GLU | 353 | 36.922 | 28.142 | −3.627 | 1.00 | 0.00 | 3A4 |
| ATOM | 2441 | CD | GLU | 353 | 36.543 | 29.540 | −4.126 | 1.00 | 0.00 | 3A4 |
| ATOM | 2442 | OE1 | GLU | 353 | 36.622 | 30.504 | −3.318 | 1.00 | 0.00 | 3A4 |
| ATOM | 2443 | OE2 | GLU | 353 | 36.171 | 29.660 | −5.325 | 1.00 | 0.00 | 3A4 |
| ATOM | 2444 | C | GLU | 353 | 34.592 | 25.102 | −2.984 | 1.00 | 0.00 | 3A4 |
| ATOM | 2445 | O | GLU | 353 | 34.078 | 25.104 | −1.864 | 1.00 | 0.00 | 3A4 |
| ATOM | 2446 | N | TYR | 354 | 33.979 | 24.537 | −4.047 | 1.00 | 0.00 | 3A4 |
| ATOM | 2447 | CA | TYR | 354 | 32.629 | 24.016 | −3.996 | 1.00 | 0.00 | 3A4 |
| ATOM | 2448 | CB | TYR | 354 | 31.928 | 24.005 | −5.369 | 1.00 | 0.00 | 3A4 |
| ATOM | 2449 | CG | TYR | 354 | 31.880 | 25.395 | −5.954 | 1.00 | 0.00 | 3A4 |
| ATOM | 2450 | CD1 | TYR | 354 | 32.962 | 25.916 | −6.691 | 1.00 | 0.00 | 3A4 |
| ATOM | 2451 | CD2 | TYR | 354 | 30.740 | 26.199 | −5.783 | 1.00 | 0.00 | 3A4 |
| ATOM | 2452 | CE1 | TYR | 354 | 32.917 | 27.212 | −7.224 | 1.00 | 0.00 | 3A4 |
| ATOM | 2453 | CE2 | TYR | 354 | 30.683 | 27.496 | −6.314 | 1.00 | 0.00 | 3A4 |
| ATOM | 2454 | CZ | TYR | 354 | 31.774 | 28.004 | −7.035 | 1.00 | 0.00 | 3A4 |
| ATOM | 2455 | OH | TYR | 354 | 31.722 | 29.310 | −7.571 | 1.00 | 0.00 | 3A4 |
| ATOM | 2456 | C | TYR | 354 | 32.592 | 22.628 | −3.428 | 1.00 | 0.00 | 3A4 |
| ATOM | 2457 | O | TYR | 354 | 31.575 | 22.220 | −2.892 | 1.00 | 0.00 | 3A4 |
| ATOM | 2458 | N | LEU | 355 | 33.710 | 21.867 | −3.466 | 1.00 | 0.00 | 3A4 |
| ATOM | 2459 | CA | LEU | 355 | 33.715 | 20.493 | −2.996 | 1.00 | 0.00 | 3A4 |
| ATOM | 2460 | CB | LEU | 355 | 34.970 | 19.771 | −3.512 | 1.00 | 0.00 | 3A4 |
| ATOM | 2461 | CG | LEU | 355 | 34.521 | 18.784 | −4.631 | 1.00 | 0.00 | 3A4 |
| ATOM | 2462 | CD1 | LEU | 355 | 35.547 | 18.525 | −5.749 | 1.00 | 0.00 | 3A4 |
| ATOM | 2463 | CD2 | LEU | 355 | 33.945 | 17.479 | −4.064 | 1.00 | 0.00 | 3A4 |
| ATOM | 2464 | C | LEU | 355 | 33.573 | 20.380 | −1.485 | 1.00 | 0.00 | 3A4 |
| ATOM | 2465 | O | LEU | 355 | 32.806 | 19.570 | −0.980 | 1.00 | 0.00 | 3A4 |
| ATOM | 2466 | N | ASP | 356 | 34.225 | 21.283 | −0.721 | 1.00 | 0.00 | 3A4 |
| ATOM | 2467 | CA | ASP | 356 | 34.070 | 21.466 | 0.714 | 1.00 | 0.00 | 3A4 |
| ATOM | 2468 | CB | ASP | 356 | 34.921 | 22.723 | 1.144 | 1.00 | 0.00 | 3A4 |
| ATOM | 2469 | CG | ASP | 356 | 35.819 | 22.461 | 2.358 | 1.00 | 0.00 | 3A4 |
| ATOM | 2470 | OD1 | ASP | 356 | 35.262 | 22.182 | 3.454 | 1.00 | 0.00 | 3A4 |
| ATOM | 2471 | OD2 | ASP | 356 | 37.067 | 22.560 | 2.214 | 1.00 | 0.00 | 3A4 |
| ATOM | 2472 | C | ASP | 356 | 32.651 | 21.700 | 1.182 | 1.00 | 0.00 | 3A4 |
| ATOM | 2473 | O | ASP | 356 | 32.180 | 21.110 | 2.149 | 1.00 | 0.00 | 3A4 |
| ATOM | 2474 | N | MET | 357 | 31.928 | 22.551 | 0.423 | 1.00 | 0.00 | 3A4 |
| ATOM | 2475 | CA | MET | 357 | 30.540 | 22.879 | 0.616 | 1.00 | 0.00 | 3A4 |
| ATOM | 2476 | CB | MET | 357 | 30.168 | 24.100 | −0.239 | 1.00 | 0.00 | 3A4 |
| ATOM | 2477 | CG | MET | 357 | 29.601 | 25.250 | 0.627 | 1.00 | 0.00 | 3A4 |
| ATOM | 2478 | SD | MET | 357 | 30.612 | 26.763 | 0.665 | 1.00 | 0.00 | 3A4 |
| ATOM | 2479 | CE | MET | 357 | 29.381 | 27.754 | 1.562 | 1.00 | 0.00 | 3A4 |
| ATOM | 2480 | C | MET | 357 | 29.622 | 21.737 | 0.289 | 1.00 | 0.00 | 3A4 |
| ATOM | 2481 | O | MET | 357 | 28.618 | 21.519 | 0.952 | 1.00 | 0.00 | 3A4 |
| ATOM | 2482 | N | VAL | 358 | 29.995 | 20.909 | −0.714 | 1.00 | 0.00 | 3A4 |
| ATOM | 2483 | CA | VAL | 358 | 29.302 | 19.693 | −1.096 | 1.00 | 0.00 | 3A4 |
| ATOM | 2484 | CB | VAL | 358 | 29.848 | 19.071 | −2.374 | 1.00 | 0.00 | 3A4 |
| ATOM | 2485 | CG1 | VAL | 358 | 29.224 | 17.667 | −2.681 | 1.00 | 0.00 | 3A4 |
| ATOM | 2486 | CG2 | VAL | 358 | 29.530 | 20.074 | −3.459 | 1.00 | 0.00 | 3A4 |
| ATOM | 2487 | C | VAL | 358 | 29.376 | 18.671 | 0.012 | 1.00 | 0.00 | 3A4 |
| ATOM | 2488 | O | VAL | 358 | 28.364 | 18.076 | 0.363 | 1.00 | 0.00 | 3A4 |
| ATOM | 2489 | N | VAL | 359 | 30.567 | 18.479 | 0.633 | 1.00 | 0.00 | 3A4 |
| ATOM | 2490 | CA | VAL | 359 | 30.783 | 17.579 | 1.758 | 1.00 | 0.00 | 3A4 |
| ATOM | 2491 | CB | VAL | 359 | 32.264 | 17.531 | 2.146 | 1.00 | 0.00 | 3A4 |
| ATOM | 2492 | CG1 | VAL | 359 | 32.553 | 16.768 | 3.459 | 1.00 | 0.00 | 3A4 |
| ATOM | 2493 | CG2 | VAL | 359 | 33.043 | 16.854 | 1.004 | 1.00 | 0.00 | 3A4 |
| ATOM | 2494 | C | VAL | 359 | 29.954 | 17.977 | 2.962 | 1.00 | 0.00 | 3A4 |
| ATOM | 2495 | O | VAL | 359 | 29.303 | 17.151 | 3.591 | 1.00 | 0.00 | 3A4 |
| ATOM | 2496 | N | ASN | 360 | 29.903 | 19.287 | 3.263 | 1.00 | 0.00 | 3A4 |
| ATOM | 2497 | CA | ASN | 360 | 29.117 | 19.830 | 4.350 | 1.00 | 0.00 | 3A4 |
| ATOM | 2498 | CB | ASN | 360 | 29.412 | 21.328 | 4.537 | 1.00 | 0.00 | 3A4 |
| ATOM | 2499 | CG | ASN | 360 | 30.795 | 21.548 | 5.189 | 1.00 | 0.00 | 3A4 |
| ATOM | 2500 | OD1 | ASN | 360 | 31.725 | 20.750 | 5.080 | 1.00 | 0.00 | 3A4 |
| ATOM | 2501 | ND2 | ASN | 360 | 30.946 | 22.706 | 5.890 | 1.00 | 0.00 | 3A4 |
| ATOM | 2502 | C | ASN | 360 | 27.624 | 19.645 | 4.147 | 1.00 | 0.00 | 3A4 |
| ATOM | 2503 | O | ASN | 360 | 26.891 | 19.274 | 5.060 | 1.00 | 0.00 | 3A4 |
| ATOM | 2504 | N | GLU | 361 | 27.149 | 19.820 | 2.890 | 1.00 | 0.00 | 3A4 |
| ATOM | 2505 | CA | GLU | 361 | 25.768 | 19.606 | 2.527 | 1.00 | 0.00 | 3A4 |
| ATOM | 2506 | CB | GLU | 361 | 25.458 | 20.211 | 1.139 | 1.00 | 0.00 | 3A4 |
| ATOM | 2507 | CG | GLU | 361 | 24.026 | 19.998 | 0.629 | 1.00 | 0.00 | 3A4 |
| ATOM | 2508 | CD | GLU | 361 | 22.935 | 20.638 | 1.489 | 1.00 | 0.00 | 3A4 |
| ATOM | 2509 | OE1 | GLU | 361 | 23.253 | 21.306 | 2.504 | 1.00 | 0.00 | 3A4 |
| ATOM | 2510 | OE2 | GLU | 361 | 21.740 | 20.442 | 1.143 | 1.00 | 0.00 | 3A4 |
| ATOM | 2511 | C | GLU | 361 | 25.372 | 18.147 | 2.553 | 1.00 | 0.00 | 3A4 |
| ATOM | 2512 | O | GLU | 361 | 24.258 | 17.809 | 2.941 | 1.00 | 0.00 | 3A4 |
| ATOM | 2513 | N | THR | 362 | 26.285 | 17.210 | 2.205 | 1.00 | 0.00 | 3A4 |
| ATOM | 2514 | CA | THR | 362 | 26.034 | 15.779 | 2.269 | 1.00 | 0.00 | 3A4 |
| ATOM | 2515 | CB | THR | 362 | 27.109 | 14.973 | 1.573 | 1.00 | 0.00 | 3A4 |
| ATOM | 2516 | OG1 | THR | 362 | 27.154 | 15.360 | 0.208 | 1.00 | 0.00 | 3A4 |
| ATOM | 2517 | CG2 | THR | 362 | 26.766 | 13.462 | 1.673 | 1.00 | 0.00 | 3A4 |
| ATOM | 2518 | C | THR | 362 | 25.886 | 15.303 | 3.702 | 1.00 | 0.00 | 3A4 |
| ATOM | 2519 | O | THR | 362 | 24.990 | 14.527 | 4.020 | 1.00 | 0.00 | 3A4 |
| ATOM | 2520 | N | LEU | 363 | 26.722 | 15.835 | 4.611 | 1.00 | 0.00 | 3A4 |
| ATOM | 2521 | CA | LEU | 363 | 26.677 | 15.555 | 6.026 | 1.00 | 0.00 | 3A4 |
| ATOM | 2522 | CB | LEU | 363 | 27.934 | 16.102 | 6.729 | 1.00 | 0.00 | 3A4 |
| ATOM | 2523 | CG | LEU | 363 | 29.216 | 15.310 | 6.428 | 1.00 | 0.00 | 3A4 |
| ATOM | 2524 | CD1 | LEU | 363 | 30.434 | 16.188 | 6.740 | 1.00 | 0.00 | 3A4 |
| ATOM | 2525 | CD2 | LEU | 363 | 29.238 | 13.981 | 7.211 | 1.00 | 0.00 | 3A4 |
| ATOM | 2526 | C | LEU | 363 | 25.448 | 16.157 | 6.698 | 1.00 | 0.00 | 3A4 |
| ATOM | 2527 | O | LEU | 363 | 24.956 | 15.614 | 7.681 | 1.00 | 0.00 | 3A4 |
| ATOM | 2528 | N | ARG | 364 | 24.890 | 17.274 | 6.163 | 1.00 | 0.00 | 3A4 |
| ATOM | 2529 | CA | ARG | 364 | 23.659 | 17.879 | 6.633 | 1.00 | 0.00 | 3A4 |
| ATOM | 2530 | CB | ARG | 364 | 23.446 | 19.303 | 6.082 | 1.00 | 0.00 | 3A4 |
| ATOM | 2531 | CG | ARG | 364 | 22.238 | 20.031 | 6.676 | 1.00 | 0.00 | 3A4 |
| ATOM | 2532 | CD | ARG | 364 | 22.212 | 21.544 | 6.417 | 1.00 | 0.00 | 3A4 |
| ATOM | 2533 | NE | ARG | 364 | 21.256 | 22.182 | 7.393 | 1.00 | 0.00 | 3A4 |
| ATOM | 2534 | CZ | ARG | 364 | 21.624 | 22.741 | 8.595 | 1.00 | 0.00 | 3A4 |
| ATOM | 2535 | NH1 | ARG | 364 | 20.663 | 23.251 | 9.418 | 1.00 | 0.00 | 3A4 |
| ATOM | 2536 | NH2 | ARG | 364 | 22.925 | 22.809 | 8.992 | 1.00 | 0.00 | 3A4 |
| ATOM | 2537 | C | ARG | 364 | 22.463 | 17.060 | 6.233 | 1.00 | 0.00 | 3A4 |
| ATOM | 2538 | O | ARG | 364 | 21.622 | 16.711 | 7.049 | 1.00 | 0.00 | 3A4 |
| ATOM | 2539 | N | LEU | 365 | 22.393 | 16.685 | 4.940 | 1.00 | 0.00 | 3A4 |
| ATOM | 2540 | CA | LEU | 365 | 21.267 | 15.979 | 4.384 | 1.00 | 0.00 | 3A4 |
| ATOM | 2541 | CB | LEU | 365 | 21.247 | 16.050 | 2.848 | 1.00 | 0.00 | 3A4 |
| ATOM | 2542 | CG | LEU | 365 | 19.879 | 16.592 | 2.326 | 1.00 | 0.00 | 3A4 |
| ATOM | 2543 | CD1 | LEU | 365 | 19.837 | 16.585 | 0.811 | 1.00 | 0.00 | 3A4 |
| ATOM | 2544 | CD2 | LEU | 365 | 18.607 | 15.874 | 2.828 | 1.00 | 0.00 | 3A4 |
| ATOM | 2545 | C | LEU | 365 | 21.135 | 14.540 | 4.810 | 1.00 | 0.00 | 3A4 |
| ATOM | 2546 | O | LEU | 365 | 20.029 | 14.082 | 5.089 | 1.00 | 0.00 | 3A4 |
| ATOM | 2547 | N | PHE | 366 | 22.252 | 13.787 | 4.904 | 1.00 | 0.00 | 3A4 |
| ATOM | 2548 | CA | PHE | 366 | 22.226 | 12.409 | 5.376 | 1.00 | 0.00 | 3A4 |
| ATOM | 2549 | CB | PHE | 366 | 22.642 | 11.385 | 4.273 | 1.00 | 0.00 | 3A4 |
| ATOM | 2550 | CG | PHE | 366 | 21.503 | 11.244 | 3.297 | 1.00 | 0.00 | 3A4 |
| ATOM | 2551 | CD1 | PHE | 366 | 21.538 | 11.861 | 2.035 | 1.00 | 0.00 | 3A4 |
| ATOM | 2552 | CD2 | PHE | 366 | 20.363 | 10.498 | 3.651 | 1.00 | 0.00 | 3A4 |
| ATOM | 2553 | CE1 | PHE | 366 | 20.455 | 11.742 | 1.150 | 1.00 | 0.00 | 3A4 |
| ATOM | 2554 | CE2 | PHE | 366 | 19.278 | 10.377 | 2.773 | 1.00 | 0.00 | 3A4 |
| ATOM | 2555 | CZ | PHE | 366 | 19.325 | 11.000 | 1.520 | 1.00 | 0.00 | 3A4 |
| ATOM | 2556 | C | PHE | 366 | 23.116 | 12.273 | 6.585 | 1.00 | 0.00 | 3A4 |
| ATOM | 2557 | O | PHE | 366 | 24.165 | 11.637 | 6.497 | 1.00 | 0.00 | 3A4 |
| ATOM | 2558 | N | PRO | 367 | 22.732 | 12.795 | 7.772 | 1.00 | 0.00 | 3A4 |
| ATOM | 2559 | CA | PRO | 367 | 23.466 | 12.674 | 9.011 | 1.00 | 0.00 | 3A4 |
| ATOM | 2560 | CD | PRO | 367 | 21.498 | 13.517 | 7.993 | 1.00 | 0.00 | 3A4 |
| ATOM | 2561 | CB | PRO | 367 | 22.894 | 13.739 | 9.935 | 1.00 | 0.00 | 3A4 |
| ATOM | 2562 | CG | PRO | 367 | 21.461 | 13.878 | 9.463 | 1.00 | 0.00 | 3A4 |
| ATOM | 2563 | C | PRO | 367 | 23.332 | 11.269 | 9.536 | 1.00 | 0.00 | 3A4 |
| ATOM | 2564 | O | PRO | 367 | 22.246 | 10.779 | 9.824 | 1.00 | 0.00 | 3A4 |
| ATOM | 2565 | N | ILE | 368 | 24.476 | 10.572 | 9.569 | 1.00 | 0.00 | 3A4 |
| ATOM | 2566 | CA | ILE | 368 | 24.602 | 9.143 | 9.718 | 1.00 | 0.00 | 3A4 |
| ATOM | 2567 | CB | ILE | 368 | 25.992 | 8.818 | 9.131 | 1.00 | 0.00 | 3A4 |
| ATOM | 2568 | CG2 | ILE | 368 | 27.160 | 9.306 | 10.024 | 1.00 | 0.00 | 3A4 |
| ATOM | 2569 | CG1 | ILE | 368 | 26.230 | 7.413 | 8.553 | 1.00 | 0.00 | 3A4 |
| ATOM | 2570 | CD | ILE | 368 | 26.421 | 6.268 | 9.546 | 1.00 | 0.00 | 3A4 |
| ATOM | 2571 | C | ILE | 368 | 24.406 | 8.654 | 11.153 | 1.00 | 0.00 | 3A4 |
| ATOM | 2572 | O | ILE | 368 | 24.024 | 7.515 | 11.392 | 1.00 | 0.00 | 3A4 |
| ATOM | 2573 | N | ALA | 369 | 24.661 | 9.530 | 12.146 | 1.00 | 0.00 | 3A4 |
| ATOM | 2574 | CA | ALA | 369 | 24.719 | 9.183 | 13.542 | 1.00 | 0.00 | 3A4 |
| ATOM | 2575 | CB | ALA | 369 | 26.067 | 9.640 | 14.141 | 1.00 | 0.00 | 3A4 |
| ATOM | 2576 | C | ALA | 369 | 23.654 | 9.859 | 14.360 | 1.00 | 0.00 | 3A4 |
| ATOM | 2577 | O | ALA | 369 | 23.267 | 10.991 | 14.104 | 1.00 | 0.00 | 3A4 |
| ATOM | 2578 | N | MET | 370 | 23.265 | 9.221 | 15.511 | 1.00 | 0.00 | 3A4 |
| ATOM | 2579 | CA | MET | 370 | 22.422 | 9.803 | 16.560 | 1.00 | 0.00 | 3A4 |
| ATOM | 2580 | CB | MET | 370 | 20.964 | 9.264 | 16.470 | 1.00 | 0.00 | 3A4 |
| ATOM | 2581 | CG | MET | 370 | 19.910 | 10.113 | 17.209 | 1.00 | 0.00 | 3A4 |
| ATOM | 2582 | SD | MET | 370 | 18.173 | 9.663 | 16.885 | 1.00 | 0.00 | 3A4 |
| ATOM | 2583 | CE | MET | 370 | 18.081 | 10.195 | 15.146 | 1.00 | 0.00 | 3A4 |
| ATOM | 2584 | C | MET | 370 | 23.062 | 9.512 | 17.921 | 1.00 | 0.00 | 3A4 |
| ATOM | 2585 | O | MET | 370 | 23.884 | 8.603 | 18.029 | 1.00 | 0.00 | 3A4 |
| ATOM | 2586 | N | ARG | 371 | 22.711 | 10.299 | 18.992 | 1.00 | 0.00 | 3A4 |
| ATOM | 2587 | CA | ARG | 371 | 23.356 | 10.277 | 20.307 | 1.00 | 0.00 | 3A4 |
| ATOM | 2588 | CB | ARG | 371 | 24.430 | 11.405 | 20.437 | 1.00 | 0.00 | 3A4 |
| ATOM | 2589 | CG | ARG | 371 | 23.944 | 12.828 | 20.099 | 1.00 | 0.00 | 3A4 |
| ATOM | 2590 | CD | ARG | 371 | 25.049 | 13.888 | 20.216 | 1.00 | 0.00 | 3A4 |
| ATOM | 2591 | NE | ARG | 371 | 24.503 | 15.208 | 19.738 | 1.00 | 0.00 | 3A4 |
| ATOM | 2592 | CZ | ARG | 371 | 25.012 | 16.428 | 20.108 | 1.00 | 0.00 | 3A4 |
| ATOM | 2593 | NH1 | ARG | 371 | 24.468 | 17.561 | 19.575 | 1.00 | 0.00 | 3A4 |
| ATOM | 2594 | NH2 | ARG | 371 | 26.049 | 16.546 | 20.988 | 1.00 | 0.00 | 3A4 |
| ATOM | 2595 | C | ARG | 371 | 22.333 | 10.392 | 21.438 | 1.00 | 0.00 | 3A4 |
| ATOM | 2596 | O | ARG | 371 | 21.181 | 10.760 | 21.216 | 1.00 | 0.00 | 3A4 |
| ATOM | 2597 | N | LEU | 372 | 22.776 | 10.045 | 22.692 | 1.00 | 0.00 | 3A4 |
| ATOM | 2598 | CA | LEU | 372 | 22.016 | 9.951 | 23.936 | 1.00 | 0.00 | 3A4 |
| ATOM | 2599 | CB | LEU | 372 | 21.964 | 8.482 | 24.512 | 1.00 | 0.00 | 3A4 |
| ATOM | 2600 | CG | LEU | 372 | 21.345 | 7.343 | 23.661 | 1.00 | 0.00 | 3A4 |
| ATOM | 2601 | CD1 | LEU | 372 | 19.908 | 7.641 | 23.227 | 1.00 | 0.00 | 3A4 |
| ATOM | 2602 | CD2 | LEU | 372 | 22.216 | 6.816 | 22.501 | 1.00 | 0.00 | 3A4 |
| ATOM | 2603 | C | LEU | 372 | 22.693 | 10.771 | 25.005 | 1.00 | 0.00 | 3A4 |
| ATOM | 2604 | O | LEU | 372 | 23.899 | 11.003 | 24.975 | 1.00 | 0.00 | 3A4 |
| ATOM | 2605 | N | GLU | 373 | 21.900 | 11.155 | 26.021 | 1.00 | 0.00 | 3A4 |
| ATOM | 2606 | CA | GLU | 373 | 22.322 | 11.807 | 27.237 | 1.00 | 0.00 | 3A4 |
| ATOM | 2607 | CB | GLU | 373 | 22.131 | 13.344 | 27.172 | 1.00 | 0.00 | 3A4 |
| ATOM | 2608 | CG | GLU | 373 | 23.197 | 14.057 | 26.329 | 1.00 | 0.00 | 3A4 |
| ATOM | 2609 | CD | GLU | 373 | 23.009 | 15.577 | 26.343 | 1.00 | 0.00 | 3A4 |
| ATOM | 2610 | OE1 | GLU | 373 | 23.722 | 16.250 | 25.553 | 1.00 | 0.00 | 3A4 |
| ATOM | 2611 | OE2 | GLU | 373 | 22.167 | 16.090 | 27.128 | 1.00 | 0.00 | 3A4 |
| ATOM | 2612 | C | GLU | 373 | 21.466 | 11.241 | 28.351 | 1.00 | 0.00 | 3A4 |
| ATOM | 2613 | O | GLU | 373 | 20.387 | 10.695 | 28.121 | 1.00 | 0.00 | 3A4 |
| ATOM | 2614 | N | ARG | 374 | 21.930 | 11.386 | 29.612 | 1.00 | 0.00 | 3A4 |
| ATOM | 2615 | CA | ARG | 374 | 21.138 | 11.043 | 30.774 | 1.00 | 0.00 | 3A4 |
| ATOM | 2616 | CB | ARG | 374 | 21.532 | 9.678 | 31.420 | 1.00 | 0.00 | 3A4 |
| ATOM | 2617 | CG | ARG | 374 | 22.371 | 9.595 | 32.715 | 1.00 | 0.00 | 3A4 |
| ATOM | 2618 | CD | ARG | 374 | 21.558 | 9.546 | 34.018 | 1.00 | 0.00 | 3A4 |
| ATOM | 2619 | NE | ARG | 374 | 22.476 | 9.916 | 35.152 | 1.00 | 0.00 | 3A4 |
| ATOM | 2620 | CZ | ARG | 374 | 22.249 | 9.595 | 36.465 | 1.00 | 0.00 | 3A4 |
| ATOM | 2621 | NH1 | ARG | 374 | 23.147 | 10.010 | 37.405 | 1.00 | 0.00 | 3A4 |
| ATOM | 2622 | NH2 | ARG | 374 | 21.156 | 8.879 | 36.860 | 1.00 | 0.00 | 3A4 |
| ATOM | 2623 | C | ARG | 374 | 21.272 | 12.200 | 31.721 | 1.00 | 0.00 | 3A4 |
| ATOM | 2624 | O | ARG | 374 | 22.360 | 12.720 | 31.947 | 1.00 | 0.00 | 3A4 |
| ATOM | 2625 | N | VAL | 375 | 20.152 | 12.629 | 32.340 | 1.00 | 0.00 | 3A4 |
| ATOM | 2626 | CA | VAL | 375 | 20.085 | 13.742 | 33.272 | 1.00 | 0.00 | 3A4 |
| ATOM | 2627 | CB | VAL | 375 | 18.651 | 14.239 | 33.344 | 1.00 | 0.00 | 3A4 |
| ATOM | 2628 | CG1 | VAL | 375 | 18.283 | 15.175 | 34.420 | 1.00 | 0.00 | 3A4 |
| ATOM | 2629 | CG2 | VAL | 375 | 18.394 | 14.888 | 31.964 | 1.00 | 0.00 | 3A4 |
| ATOM | 2630 | C | VAL | 375 | 20.606 | 13.314 | 34.625 | 1.00 | 0.00 | 3A4 |
| ATOM | 2631 | O | VAL | 375 | 20.173 | 12.325 | 35.197 | 1.00 | 0.00 | 3A4 |
| ATOM | 2632 | N | CYS | 376 | 21.593 | 14.058 | 35.166 | 1.00 | 0.00 | 3A4 |
| ATOM | 2633 | CA | CYS | 376 | 22.269 | 13.722 | 36.404 | 1.00 | 0.00 | 3A4 |
| ATOM | 2634 | CB | CYS | 376 | 23.708 | 14.284 | 36.408 | 1.00 | 0.00 | 3A4 |
| ATOM | 2635 | SG | CYS | 376 | 24.721 | 13.423 | 35.165 | 1.00 | 0.00 | 3A4 |
| ATOM | 2636 | C | CYS | 376 | 21.525 | 14.230 | 37.618 | 1.00 | 0.00 | 3A4 |
| ATOM | 2637 | O | CYS | 376 | 21.699 | 13.713 | 38.720 | 1.00 | 0.00 | 3A4 |
| ATOM | 2638 | N | LYS | 377 | 20.691 | 15.268 | 37.426 | 1.00 | 0.00 | 3A4 |
| ATOM | 2639 | CA | LYS | 377 | 19.982 | 15.944 | 38.480 | 1.00 | 0.00 | 3A4 |
| ATOM | 2640 | CB | LYS | 377 | 20.773 | 17.167 | 38.996 | 1.00 | 0.00 | 3A4 |
| ATOM | 2641 | CG | LYS | 377 | 22.008 | 16.876 | 39.867 | 1.00 | 0.00 | 3A4 |
| ATOM | 2642 | CD | LYS | 377 | 21.695 | 16.179 | 41.198 | 1.00 | 0.00 | 3A4 |
| ATOM | 2643 | CE | LYS | 377 | 22.947 | 15.957 | 42.056 | 1.00 | 0.00 | 3A4 |
| ATOM | 2644 | NZ | LYS | 377 | 22.604 | 15.293 | 43.336 | 1.00 | 0.00 | 3A4 |
| ATOM | 2645 | C | LYS | 377 | 18.735 | 16.448 | 37.827 | 1.00 | 0.00 | 3A4 |
| ATOM | 2646 | O | LYS | 377 | 18.689 | 16.603 | 36.617 | 1.00 | 0.00 | 3A4 |
| ATOM | 2647 | N | LYS | 378 | 17.690 | 16.839 | 38.593 | 1.00 | 0.00 | 3A4 |
| ATOM | 2648 | CA | LYS | 378 | 16.465 | 17.414 | 38.063 | 1.00 | 0.00 | 3A4 |
| ATOM | 2649 | CB | LYS | 378 | 15.419 | 17.607 | 39.177 | 1.00 | 0.00 | 3A4 |
| ATOM | 2650 | CG | LYS | 378 | 15.088 | 16.287 | 39.895 | 1.00 | 0.00 | 3A4 |
| ATOM | 2651 | CD | LYS | 378 | 14.017 | 16.418 | 40.984 | 1.00 | 0.00 | 3A4 |
| ATOM | 2652 | CE | LYS | 378 | 13.613 | 15.082 | 41.625 | 1.00 | 0.00 | 3A4 |
| ATOM | 2653 | NZ | LYS | 378 | 14.747 | 14.462 | 42.352 | 1.00 | 0.00 | 3A4 |
| ATOM | 2654 | C | LYS | 378 | 16.741 | 18.741 | 37.382 | 1.00 | 0.00 | 3A4 |
| ATOM | 2655 | O | LYS | 378 | 17.545 | 19.524 | 37.877 | 1.00 | 0.00 | 3A4 |
| ATOM | 2656 | N | ASP | 379 | 16.172 | 18.960 | 36.183 | 1.00 | 0.00 | 3A4 |
| ATOM | 2657 | CA | ASP | 379 | 16.483 | 20.125 | 35.394 | 1.00 | 0.00 | 3A4 |
| ATOM | 2658 | CB | ASP | 379 | 17.706 | 19.925 | 34.443 | 1.00 | 0.00 | 3A4 |
| ATOM | 2659 | CG | ASP | 379 | 17.527 | 19.059 | 33.183 | 1.00 | 0.00 | 3A4 |
| ATOM | 2660 | OD1 | ASP | 379 | 17.455 | 17.817 | 33.330 | 1.00 | 0.00 | 3A4 |
| ATOM | 2661 | OD2 | ASP | 379 | 17.475 | 19.634 | 32.063 | 1.00 | 0.00 | 3A4 |
| ATOM | 2662 | C | ASP | 379 | 15.250 | 20.543 | 34.652 | 1.00 | 0.00 | 3A4 |
| ATOM | 2663 | O | ASP | 379 | 14.333 | 19.776 | 34.415 | 1.00 | 0.00 | 3A4 |
| ATOM | 2664 | N | VAL | 380 | 15.216 | 21.819 | 34.247 | 1.00 | 0.00 | 3A4 |
| ATOM | 2665 | CA | VAL | 380 | 14.098 | 22.398 | 33.548 | 1.00 | 0.00 | 3A4 |
| ATOM | 2666 | CB | VAL | 380 | 13.450 | 23.491 | 34.376 | 1.00 | 0.00 | 3A4 |
| ATOM | 2667 | CG1 | VAL | 380 | 12.212 | 24.040 | 33.665 | 1.00 | 0.00 | 3A4 |
| ATOM | 2668 | CG2 | VAL | 380 | 13.057 | 22.943 | 35.763 | 1.00 | 0.00 | 3A4 |
| ATOM | 2669 | C | VAL | 380 | 14.659 | 22.869 | 32.227 | 1.00 | 0.00 | 3A4 |
| ATOM | 2670 | O | VAL | 380 | 15.521 | 23.743 | 32.201 | 1.00 | 0.00 | 3A4 |
| ATOM | 2671 | N | GLU | 381 | 14.219 | 22.259 | 31.107 | 1.00 | 0.00 | 3A4 |
| ATOM | 2672 | CA | GLU | 381 | 14.653 | 22.560 | 29.760 | 1.00 | 0.00 | 3A4 |
| ATOM | 2673 | CB | GLU | 381 | 15.044 | 21.298 | 28.989 | 1.00 | 0.00 | 3A4 |
| ATOM | 2674 | CG | GLU | 381 | 15.995 | 21.611 | 27.810 | 1.00 | 0.00 | 3A4 |
| ATOM | 2675 | CD | GLU | 381 | 16.331 | 20.375 | 26.965 | 1.00 | 0.00 | 3A4 |
| ATOM | 2676 | OE1 | GLU | 381 | 17.063 | 20.564 | 25.958 | 1.00 | 0.00 | 3A4 |
| ATOM | 2677 | OE2 | GLU | 381 | 15.873 | 19.246 | 27.284 | 1.00 | 0.00 | 3A4 |
| ATOM | 2678 | C | GLU | 381 | 13.495 | 23.231 | 29.052 | 1.00 | 0.00 | 3A4 |
| ATOM | 2679 | O | GLU | 381 | 12.355 | 22.802 | 29.042 | 1.00 | 0.00 | 3A4 |
| ATOM | 2680 | N | ILE | 382 | 13.715 | 24.405 | 28.468 | 1.00 | 0.00 | 3A4 |
| ATOM | 2681 | CA | ILE | 382 | 12.690 | 25.313 | 27.960 | 1.00 | 0.00 | 3A4 |
| ATOM | 2682 | CB | ILE | 382 | 11.956 | 24.945 | 26.638 | 1.00 | 0.00 | 3A4 |
| ATOM | 2683 | CG2 | ILE | 382 | 10.525 | 25.549 | 26.472 | 1.00 | 0.00 | 3A4 |
| ATOM | 2684 | CG1 | ILE | 382 | 12.874 | 25.434 | 25.467 | 1.00 | 0.00 | 3A4 |
| ATOM | 2685 | CD | ILE | 382 | 12.229 | 25.497 | 24.082 | 1.00 | 0.00 | 3A4 |
| ATOM | 2686 | C | ILE | 382 | 11.919 | 25.809 | 29.163 | 1.00 | 0.00 | 3A4 |
| ATOM | 2687 | O | ILE | 382 | 12.497 | 26.469 | 29.990 | 1.00 | 0.00 | 3A4 |
| ATOM | 2688 | N | ASN | 383 | 10.722 | 25.435 | 29.490 | 1.00 | 0.00 | 3A4 |
| ATOM | 2689 | CA | ASN | 383 | 10.349 | 25.838 | 30.866 | 1.00 | 0.00 | 3A4 |
| ATOM | 2690 | CB | ASN | 383 | 9.806 | 27.330 | 30.959 | 1.00 | 0.00 | 3A4 |
| ATOM | 2691 | CG | ASN | 383 | 9.693 | 27.860 | 32.407 | 1.00 | 0.00 | 3A4 |
| ATOM | 2692 | OD1 | ASN | 383 | 8.596 | 28.169 | 32.869 | 1.00 | 0.00 | 3A4 |
| ATOM | 2693 | ND2 | ASN | 383 | 10.843 | 27.967 | 33.129 | 1.00 | 0.00 | 3A4 |
| ATOM | 2694 | C | ASN | 383 | 9.349 | 24.945 | 31.284 | 1.00 | 0.00 | 3A4 |
| ATOM | 2695 | O | ASN | 383 | 8.160 | 25.107 | 30.993 | 1.00 | 0.00 | 3A4 |
| ATOM | 2696 | N | GLY | 384 | 9.485 | 23.892 | 32.102 | 1.00 | 0.00 | 3A4 |
| ATOM | 2697 | CA | GLY | 384 | 8.315 | 22.984 | 32.156 | 1.00 | 0.00 | 3A4 |
| ATOM | 2698 | C | GLY | 384 | 8.276 | 22.292 | 30.798 | 1.00 | 0.00 | 3A4 |
| ATOM | 2699 | O | GLY | 384 | 7.285 | 21.764 | 30.297 | 1.00 | 0.00 | 3A4 |
| ATOM | 2700 | N | MET | 385 | 9.600 | 22.252 | 30.331 | 1.00 | 0.00 | 3A4 |
| ATOM | 2701 | CA | MET | 385 | 9.904 | 20.858 | 30.491 | 1.00 | 0.00 | 3A4 |
| ATOM | 2702 | CB | MET | 385 | 10.648 | 20.273 | 29.245 | 1.00 | 0.00 | 3A4 |
| ATOM | 2703 | CG | MET | 385 | 10.078 | 18.932 | 28.749 | 1.00 | 0.00 | 3A4 |
| ATOM | 2704 | SD | MET | 385 | 10.271 | 17.522 | 29.885 | 1.00 | 0.00 | 3A4 |
| ATOM | 2705 | CE | MET | 385 | 8.674 | 16.747 | 29.504 | 1.00 | 0.00 | 3A4 |
| ATOM | 2706 | C | MET | 385 | 10.673 | 20.574 | 31.772 | 1.00 | 0.00 | 3A4 |
| ATOM | 2707 | O | MET | 385 | 11.823 | 20.957 | 31.867 | 1.00 | 0.00 | 3A4 |
| ATOM | 2708 | N | PHE | 386 | 10.077 | 19.903 | 32.767 | 1.00 | 0.00 | 3A4 |
| ATOM | 2709 | CA | PHE | 386 | 10.786 | 19.416 | 33.933 | 1.00 | 0.00 | 3A4 |
| ATOM | 2710 | CB | PHE | 386 | 9.854 | 19.471 | 35.160 | 1.00 | 0.00 | 3A4 |
| ATOM | 2711 | CG | PHE | 386 | 10.561 | 19.327 | 36.492 | 1.00 | 0.00 | 3A4 |
| ATOM | 2712 | CD1 | PHE | 386 | 10.905 | 20.467 | 37.242 | 1.00 | 0.00 | 3A4 |
| ATOM | 2713 | CD2 | PHE | 386 | 10.861 | 18.057 | 37.026 | 1.00 | 0.00 | 3A4 |
| ATOM | 2714 | CE1 | PHE | 386 | 11.556 | 20.349 | 38.477 | 1.00 | 0.00 | 3A4 |
| ATOM | 2715 | CE2 | PHE | 386 | 11.512 | 17.931 | 38.260 | 1.00 | 0.00 | 3A4 |
| ATOM | 2716 | CZ | PHE | 386 | 11.859 | 19.080 | 38.985 | 1.00 | 0.00 | 3A4 |
| ATOM | 2717 | C | PHE | 386 | 11.245 | 17.997 | 33.650 | 1.00 | 0.00 | 3A4 |
| ATOM | 2718 | O | PHE | 386 | 10.434 | 17.097 | 33.442 | 1.00 | 0.00 | 3A4 |
| ATOM | 2719 | N | ILE | 387 | 12.579 | 17.797 | 33.622 | 1.00 | 0.00 | 3A4 |
| ATOM | 2720 | CA | ILE | 387 | 13.234 | 16.542 | 33.348 | 1.00 | 0.00 | 3A4 |
| ATOM | 2721 | CB | ILE | 387 | 14.381 | 16.682 | 32.335 | 1.00 | 0.00 | 3A4 |
| ATOM | 2722 | CG2 | ILE | 387 | 14.760 | 15.256 | 31.874 | 1.00 | 0.00 | 3A4 |
| ATOM | 2723 | CG1 | ILE | 387 | 13.940 | 17.566 | 31.142 | 1.00 | 0.00 | 3A4 |
| ATOM | 2724 | CD | ILE | 387 | 14.996 | 17.732 | 30.052 | 1.00 | 0.00 | 3A4 |
| ATOM | 2725 | C | ILE | 387 | 13.712 | 16.037 | 34.699 | 1.00 | 0.00 | 3A4 |
| ATOM | 2726 | O | ILE | 387 | 14.542 | 16.701 | 35.311 | 1.00 | 0.00 | 3A4 |
| ATOM | 2727 | N | PRO | 388 | 13.230 | 14.898 | 35.231 | 1.00 | 0.00 | 3A4 |
| ATOM | 2728 | CA | PRO | 388 | 13.741 | 14.297 | 36.449 | 1.00 | 0.00 | 3A4 |
| ATOM | 2729 | CD | PRO | 388 | 11.827 | 14.522 | 35.038 | 1.00 | 0.00 | 3A4 |
| ATOM | 2730 | CB | PRO | 388 | 12.685 | 13.264 | 36.864 | 1.00 | 0.00 | 3A4 |
| ATOM | 2731 | CG | PRO | 388 | 11.381 | 13.833 | 36.327 | 1.00 | 0.00 | 3A4 |
| ATOM | 2732 | C | PRO | 388 | 15.090 | 13.656 | 36.297 | 1.00 | 0.00 | 3A4 |
| ATOM | 2733 | O | PRO | 388 | 15.481 | 13.246 | 35.212 | 1.00 | 0.00 | 3A4 |
| ATOM | 2734 | N | LYS | 389 | 15.825 | 13.516 | 37.416 | 1.00 | 0.00 | 3A4 |
| ATOM | 2735 | CA | LYS | 389 | 17.102 | 12.839 | 37.479 | 1.00 | 0.00 | 3A4 |
| ATOM | 2736 | CB | LYS | 389 | 17.661 | 12.891 | 38.918 | 1.00 | 0.00 | 3A4 |
| ATOM | 2737 | CG | LYS | 389 | 16.754 | 12.453 | 40.092 | 1.00 | 0.00 | 3A4 |
| ATOM | 2738 | CD | LYS | 389 | 16.878 | 10.984 | 40.532 | 1.00 | 0.00 | 3A4 |
| ATOM | 2739 | CE | LYS | 389 | 16.079 | 10.653 | 41.802 | 1.00 | 0.00 | 3A4 |
| ATOM | 2740 | NZ | LYS | 389 | 14.624 | 10.849 | 41.589 | 1.00 | 0.00 | 3A4 |
| ATOM | 2741 | C | LYS | 389 | 16.984 | 11.412 | 37.009 | 1.00 | 0.00 | 3A4 |
| ATOM | 2742 | O | LYS | 389 | 15.997 | 10.745 | 37.301 | 1.00 | 0.00 | 3A4 |
| ATOM | 2743 | N | GLY | 390 | 17.932 | 10.973 | 36.172 | 1.00 | 0.00 | 3A4 |
| ATOM | 2744 | CA | GLY | 390 | 17.963 | 9.654 | 35.593 | 1.00 | 0.00 | 3A4 |
| ATOM | 2745 | C | GLY | 390 | 17.174 | 9.470 | 34.332 | 1.00 | 0.00 | 3A4 |
| ATOM | 2746 | O | GLY | 390 | 17.046 | 8.351 | 33.839 | 1.00 | 0.00 | 3A4 |
| ATOM | 2747 | N | TRP | 391 | 16.619 | 10.571 | 33.769 | 1.00 | 0.00 | 3A4 |
| ATOM | 2748 | CA | TRP | 391 | 15.850 | 10.544 | 32.546 | 1.00 | 0.00 | 3A4 |
| ATOM | 2749 | CB | TRP | 391 | 14.930 | 11.760 | 32.448 | 1.00 | 0.00 | 3A4 |
| ATOM | 2750 | CG | TRP | 391 | 13.571 | 11.608 | 33.100 | 1.00 | 0.00 | 3A4 |
| ATOM | 2751 | CD2 | TRP | 391 | 13.050 | 10.792 | 34.187 | 1.00 | 0.00 | 3A4 |
| ATOM | 2752 | CD1 | TRP | 391 | 12.458 | 12.068 | 32.439 | 1.00 | 0.00 | 3A4 |
| ATOM | 2753 | NE1 | TRP | 391 | 11.305 | 11.618 | 33.025 | 1.00 | 0.00 | 3A4 |
| ATOM | 2754 | CE2 | TRP | 391 | 11.634 | 10.839 | 34.102 | 1.00 | 0.00 | 3A4 |
| ATOM | 2755 | CE3 | TRP | 391 | 13.655 | 10.032 | 35.188 | 1.00 | 0.00 | 3A4 |
| ATOM | 2756 | CZ2 | TRP | 391 | 10.825 | 10.153 | 35.005 | 1.00 | 0.00 | 3A4 |
| ATOM | 2757 | CZ3 | TRP | 391 | 12.843 | 9.345 | 36.102 | 1.00 | 0.00 | 3A4 |
| ATOM | 2758 | CH2 | TRP | 391 | 11.446 | 9.404 | 36.011 | 1.00 | 0.00 | 3A4 |
| ATOM | 2759 | C | TRP | 391 | 16.783 | 10.563 | 31.369 | 1.00 | 0.00 | 3A4 |
| ATOM | 2760 | O | TRP | 391 | 17.769 | 11.289 | 31.371 | 1.00 | 0.00 | 3A4 |
| ATOM | 2761 | N | VAL | 392 | 16.466 | 9.736 | 30.350 | 1.00 | 0.00 | 3A4 |
| ATOM | 2762 | CA | VAL | 392 | 17.263 | 9.564 | 29.159 | 1.00 | 0.00 | 3A4 |
| ATOM | 2763 | CB | VAL | 392 | 17.270 | 8.105 | 28.700 | 1.00 | 0.00 | 3A4 |
| ATOM | 2764 | CG1 | VAL | 392 | 17.957 | 7.898 | 27.322 | 1.00 | 0.00 | 3A4 |
| ATOM | 2765 | CG2 | VAL | 392 | 17.984 | 7.270 | 29.787 | 1.00 | 0.00 | 3A4 |
| ATOM | 2766 | C | VAL | 392 | 16.723 | 10.497 | 28.110 | 1.00 | 0.00 | 3A4 |
| ATOM | 2767 | O | VAL | 392 | 15.519 | 10.598 | 27.908 | 1.00 | 0.00 | 3A4 |
| ATOM | 2768 | N | VAL | 393 | 17.636 | 11.234 | 27.453 | 1.00 | 0.00 | 3A4 |
| ATOM | 2769 | CA | VAL | 393 | 17.333 | 12.226 | 26.455 | 1.00 | 0.00 | 3A4 |
| ATOM | 2770 | CB | VAL | 393 | 17.828 | 13.610 | 26.868 | 1.00 | 0.00 | 3A4 |
| ATOM | 2771 | CG1 | VAL | 393 | 17.556 | 14.652 | 25.780 | 1.00 | 0.00 | 3A4 |
| ATOM | 2772 | CG2 | VAL | 393 | 17.145 | 14.023 | 28.194 | 1.00 | 0.00 | 3A4 |
| ATOM | 2773 | C | VAL | 393 | 17.993 | 11.735 | 25.192 | 1.00 | 0.00 | 3A4 |
| ATOM | 2774 | O | VAL | 393 | 19.147 | 11.327 | 25.196 | 1.00 | 0.00 | 3A4 |
| ATOM | 2775 | N | MET | 394 | 17.251 | 11.776 | 24.072 | 1.00 | 0.00 | 3A4 |
| ATOM | 2776 | CA | MET | 394 | 17.726 | 11.439 | 22.757 | 1.00 | 0.00 | 3A4 |
| ATOM | 2777 | CB | MET | 394 | 16.799 | 10.393 | 22.065 | 1.00 | 0.00 | 3A4 |
| ATOM | 2778 | CG | MET | 394 | 16.558 | 9.106 | 22.860 | 1.00 | 0.00 | 3A4 |
| ATOM | 2779 | SD | MET | 394 | 15.673 | 7.800 | 21.946 | 1.00 | 0.00 | 3A4 |
| ATOM | 2780 | CE | MET | 394 | 16.910 | 7.426 | 20.666 | 1.00 | 0.00 | 3A4 |
| ATOM | 2781 | C | MET | 394 | 17.772 | 12.724 | 21.991 | 1.00 | 0.00 | 3A4 |
| ATOM | 2782 | O | MET | 394 | 16.799 | 13.464 | 21.947 | 1.00 | 0.00 | 3A4 |
| ATOM | 2783 | N | ILE | 395 | 18.916 | 13.023 | 21.347 | 1.00 | 0.00 | 3A4 |
| ATOM | 2784 | CA | ILE | 395 | 19.139 | 14.208 | 20.554 | 1.00 | 0.00 | 3A4 |
| ATOM | 2785 | CB | ILE | 395 | 20.476 | 14.837 | 20.956 | 1.00 | 0.00 | 3A4 |
| ATOM | 2786 | CG2 | ILE | 395 | 21.095 | 15.840 | 19.965 | 1.00 | 0.00 | 3A4 |
| ATOM | 2787 | CG1 | ILE | 395 | 20.413 | 15.442 | 22.374 | 1.00 | 0.00 | 3A4 |
| ATOM | 2788 | CD | ILE | 395 | 20.802 | 14.496 | 23.518 | 1.00 | 0.00 | 3A4 |
| ATOM | 2789 | C | ILE | 395 | 19.146 | 13.747 | 19.102 | 1.00 | 0.00 | 3A4 |
| ATOM | 2790 | O | ILE | 395 | 20.160 | 13.239 | 18.627 | 1.00 | 0.00 | 3A4 |
| ATOM | 2791 | N | PRO | 396 | 18.048 | 13.907 | 18.342 | 1.00 | 0.00 | 3A4 |
| ATOM | 2792 | CA | PRO | 396 | 18.027 | 13.792 | 16.906 | 1.00 | 0.00 | 3A4 |
| ATOM | 2793 | CD | PRO | 396 | 16.755 | 13.448 | 18.870 | 1.00 | 0.00 | 3A4 |
| ATOM | 2794 | CB | PRO | 396 | 16.531 | 13.829 | 16.546 | 1.00 | 0.00 | 3A4 |
| ATOM | 2795 | CG | PRO | 396 | 15.857 | 13.097 | 17.687 | 1.00 | 0.00 | 3A4 |
| ATOM | 2796 | C | PRO | 396 | 18.776 | 14.889 | 16.173 | 1.00 | 0.00 | 3A4 |
| ATOM | 2797 | O | PRO | 396 | 18.239 | 15.950 | 15.861 | 1.00 | 0.00 | 3A4 |
| ATOM | 2798 | N | SER | 397 | 20.048 | 14.601 | 15.825 | 1.00 | 0.00 | 3A4 |
| ATOM | 2799 | CA | SER | 397 | 20.904 | 15.470 | 15.055 | 1.00 | 0.00 | 3A4 |
| ATOM | 2800 | CB | SER | 397 | 22.360 | 14.989 | 15.079 | 1.00 | 0.00 | 3A4 |
| ATOM | 2801 | OG | SER | 397 | 22.500 | 13.601 | 14.800 | 1.00 | 0.00 | 3A4 |
| ATOM | 2802 | C | SER | 397 | 20.417 | 15.595 | 13.635 | 1.00 | 0.00 | 3A4 |
| ATOM | 2803 | O | SER | 397 | 20.522 | 16.640 | 13.023 | 1.00 | 0.00 | 3A4 |
| ATOM | 2804 | N | TYR | 398 | 19.766 | 14.539 | 13.106 | 1.00 | 0.00 | 3A4 |
| ATOM | 2805 | CA | TYR | 398 | 19.026 | 14.496 | 11.862 | 1.00 | 0.00 | 3A4 |
| ATOM | 2806 | CB | TYR | 398 | 18.323 | 13.079 | 11.799 | 1.00 | 0.00 | 3A4 |
| ATOM | 2807 | CG | TYR | 398 | 18.447 | 12.353 | 10.483 | 1.00 | 0.00 | 3A4 |
| ATOM | 2808 | CD1 | TYR | 398 | 19.004 | 11.056 | 10.456 | 1.00 | 0.00 | 3A4 |
| ATOM | 2809 | CD2 | TYR | 398 | 18.041 | 12.935 | 9.266 | 1.00 | 0.00 | 3A4 |
| ATOM | 2810 | CE1 | TYR | 398 | 19.153 | 10.357 | 9.251 | 1.00 | 0.00 | 3A4 |
| ATOM | 2811 | CE2 | TYR | 398 | 18.216 | 12.249 | 8.058 | 1.00 | 0.00 | 3A4 |
| ATOM | 2812 | CZ | TYR | 398 | 18.763 | 10.958 | 8.048 | 1.00 | 0.00 | 3A4 |
| ATOM | 2813 | OH | TYR | 398 | 18.924 | 10.266 | 6.829 | 1.00 | 0.00 | 3A4 |
| ATOM | 2814 | C | TYR | 398 | 17.923 | 15.540 | 11.741 | 1.00 | 0.00 | 3A4 |
| ATOM | 2815 | O | TYR | 398 | 17.730 | 16.207 | 10.728 | 1.00 | 0.00 | 3A4 |
| ATOM | 2816 | N | ALA | 399 | 17.184 | 15.730 | 12.853 | 1.00 | 0.00 | 3A4 |
| ATOM | 2817 | CA | ALA | 399 | 16.116 | 16.686 | 12.969 | 1.00 | 0.00 | 3A4 |
| ATOM | 2818 | CB | ALA | 399 | 15.263 | 16.409 | 14.206 | 1.00 | 0.00 | 3A4 |
| ATOM | 2819 | C | ALA | 399 | 16.594 | 18.122 | 13.028 | 1.00 | 0.00 | 3A4 |
| ATOM | 2820 | O | ALA | 399 | 15.939 | 19.008 | 12.501 | 1.00 | 0.00 | 3A4 |
| ATOM | 2821 | N | LEU | 400 | 17.795 | 18.377 | 13.599 | 1.00 | 0.00 | 3A4 |
| ATOM | 2822 | CA | LEU | 400 | 18.426 | 19.687 | 13.645 | 1.00 | 0.00 | 3A4 |
| ATOM | 2823 | CB | LEU | 400 | 19.762 | 19.647 | 14.448 | 1.00 | 0.00 | 3A4 |
| ATOM | 2824 | CG | LEU | 400 | 19.694 | 19.664 | 15.956 | 1.00 | 0.00 | 3A4 |
| ATOM | 2825 | CD1 | LEU | 400 | 20.663 | 20.773 | 16.433 | 1.00 | 0.00 | 3A4 |
| ATOM | 2826 | CD2 | LEU | 400 | 18.244 | 19.759 | 16.433 | 1.00 | 0.00 | 3A4 |
| ATOM | 2827 | C | LEU | 400 | 18.832 | 20.202 | 12.285 | 1.00 | 0.00 | 3A4 |
| ATOM | 2828 | O | LEU | 400 | 18.639 | 21.368 | 11.954 | 1.00 | 0.00 | 3A4 |
| ATOM | 2829 | N | HIS | 401 | 19.402 | 19.298 | 11.465 | 1.00 | 0.00 | 3A4 |
| ATOM | 2830 | CA | HIS | 401 | 19.852 | 19.556 | 10.124 | 1.00 | 0.00 | 3A4 |
| ATOM | 2831 | ND1 | HIS | 401 | 22.332 | 16.943 | 10.714 | 1.00 | 0.00 | 3A4 |
| ATOM | 2832 | CG | HIS | 401 | 21.917 | 18.117 | 10.143 | 1.00 | 0.00 | 3A4 |
| ATOM | 2833 | CB | HIS | 401 | 20.572 | 18.319 | 9.559 | 1.00 | 0.00 | 3A4 |
| ATOM | 2834 | NE2 | HIS | 401 | 24.123 | 18.219 | 10.505 | 1.00 | 0.00 | 3A4 |
| ATOM | 2835 | CD2 | HIS | 401 | 23.023 | 18.893 | 10.033 | 1.00 | 0.00 | 3A4 |
| ATOM | 2836 | CE1 | HIS | 401 | 23.659 | 17.057 | 10.914 | 1.00 | 0.00 | 3A4 |
| ATOM | 2837 | C | HIS | 401 | 18.745 | 19.836 | 9.125 | 1.00 | 0.00 | 3A4 |
| ATOM | 2838 | O | HIS | 401 | 18.970 | 20.398 | 8.056 | 1.00 | 0.00 | 3A4 |
| ATOM | 2839 | N | ARG | 402 | 17.513 | 19.410 | 9.474 | 1.00 | 0.00 | 3A4 |
| ATOM | 2840 | CA | ARG | 402 | 16.350 | 19.558 | 8.644 | 1.00 | 0.00 | 3A4 |
| ATOM | 2841 | CB | ARG | 402 | 15.632 | 18.220 | 8.410 | 1.00 | 0.00 | 3A4 |
| ATOM | 2842 | CG | ARG | 402 | 16.476 | 17.220 | 7.607 | 1.00 | 0.00 | 3A4 |
| ATOM | 2843 | CD | ARG | 402 | 15.663 | 15.992 | 7.176 | 1.00 | 0.00 | 3A4 |
| ATOM | 2844 | NE | ARG | 402 | 16.519 | 15.105 | 6.308 | 1.00 | 0.00 | 3A4 |
| ATOM | 2845 | CZ | ARG | 402 | 16.028 | 14.373 | 5.254 | 1.00 | 0.00 | 3A4 |
| ATOM | 2846 | NH1 | ARG | 402 | 16.860 | 13.518 | 4.593 | 1.00 | 0.00 | 3A4 |
| ATOM | 2847 | NH2 | ARG | 402 | 14.725 | 14.465 | 4.853 | 1.00 | 0.00 | 3A4 |
| ATOM | 2848 | C | ARG | 402 | 15.378 | 20.553 | 9.211 | 1.00 | 0.00 | 3A4 |
| ATOM | 2849 | O | ARG | 402 | 14.224 | 20.625 | 8.790 | 1.00 | 0.00 | 3A4 |
| ATOM | 2850 | N | ASP | 403 | 15.839 | 21.391 | 10.171 | 1.00 | 0.00 | 3A4 |
| ATOM | 2851 | CA | ASP | 403 | 15.031 | 22.422 | 10.772 | 1.00 | 0.00 | 3A4 |
| ATOM | 2852 | CB | ASP | 403 | 15.642 | 22.973 | 12.085 | 1.00 | 0.00 | 3A4 |
| ATOM | 2853 | CG | ASP | 403 | 14.530 | 23.734 | 12.847 | 1.00 | 0.00 | 3A4 |
| ATOM | 2854 | OD1 | ASP | 403 | 14.116 | 24.806 | 12.394 | 1.00 | 0.00 | 3A4 |
| ATOM | 2855 | OD2 | ASP | 403 | 13.981 | 23.160 | 13.823 | 1.00 | 0.00 | 3A4 |
| ATOM | 2856 | C | ASP | 403 | 14.822 | 23.549 | 9.771 | 1.00 | 0.00 | 3A4 |
| ATOM | 2857 | O | ASP | 403 | 15.805 | 24.148 | 9.348 | 1.00 | 0.00 | 3A4 |
| ATOM | 2858 | N | PRO | 404 | 13.575 | 23.872 | 9.387 | 1.00 | 0.00 | 3A4 |
| ATOM | 2859 | CA | PRO | 404 | 13.256 | 24.866 | 8.377 | 1.00 | 0.00 | 3A4 |
| ATOM | 2860 | CD | PRO | 404 | 12.350 | 23.288 | 9.945 | 1.00 | 0.00 | 3A4 |
| ATOM | 2861 | CB | PRO | 404 | 11.749 | 24.727 | 8.133 | 1.00 | 0.00 | 3A4 |
| ATOM | 2862 | CG | PRO | 404 | 11.195 | 24.161 | 9.445 | 1.00 | 0.00 | 3A4 |
| ATOM | 2863 | C | PRO | 404 | 13.582 | 26.287 | 8.784 | 1.00 | 0.00 | 3A4 |
| ATOM | 2864 | O | PRO | 404 | 13.786 | 27.122 | 7.915 | 1.00 | 0.00 | 3A4 |
| ATOM | 2865 | N | LYS | 405 | 13.702 | 26.594 | 10.092 | 1.00 | 0.00 | 3A4 |
| ATOM | 2866 | CA | LYS | 405 | 14.102 | 27.890 | 10.588 | 1.00 | 0.00 | 3A4 |
| ATOM | 2867 | CB | LYS | 405 | 13.708 | 28.082 | 12.078 | 1.00 | 0.00 | 3A4 |
| ATOM | 2868 | CG | LYS | 405 | 12.230 | 27.779 | 12.361 | 1.00 | 0.00 | 3A4 |
| ATOM | 2869 | CD | LYS | 405 | 11.862 | 27.931 | 13.843 | 1.00 | 0.00 | 3A4 |
| ATOM | 2870 | CE | LYS | 405 | 10.437 | 27.465 | 14.181 | 1.00 | 0.00 | 3A4 |
| ATOM | 2871 | NZ | LYS | 405 | 9.420 | 28.274 | 13.467 | 1.00 | 0.00 | 3A4 |
| ATOM | 2872 | C | LYS | 405 | 15.589 | 28.111 | 10.451 | 1.00 | 0.00 | 3A4 |
| ATOM | 2873 | O | LYS | 405 | 16.040 | 29.247 | 10.435 | 1.00 | 0.00 | 3A4 |
| ATOM | 2874 | N | TYR | 406 | 16.387 | 27.031 | 10.326 | 1.00 | 0.00 | 3A4 |
| ATOM | 2875 | CA | TYR | 406 | 17.828 | 27.082 | 10.196 | 1.00 | 0.00 | 3A4 |
| ATOM | 2876 | CB | TYR | 406 | 18.501 | 25.978 | 11.073 | 1.00 | 0.00 | 3A4 |
| ATOM | 2877 | CG | TYR | 406 | 18.818 | 26.481 | 12.466 | 1.00 | 0.00 | 3A4 |
| ATOM | 2878 | CD1 | TYR | 406 | 17.845 | 27.049 | 13.316 | 1.00 | 0.00 | 3A4 |
| ATOM | 2879 | CD2 | TYR | 406 | 20.123 | 26.324 | 12.973 | 1.00 | 0.00 | 3A4 |
| ATOM | 2880 | CE1 | TYR | 406 | 18.180 | 27.502 | 14.601 | 1.00 | 0.00 | 3A4 |
| ATOM | 2881 | CE2 | TYR | 406 | 20.465 | 26.758 | 14.261 | 1.00 | 0.00 | 3A4 |
| ATOM | 2882 | CZ | TYR | 406 | 19.493 | 27.354 | 15.076 | 1.00 | 0.00 | 3A4 |
| ATOM | 2883 | OH | TYR | 406 | 19.831 | 27.781 | 16.379 | 1.00 | 0.00 | 3A4 |
| ATOM | 2884 | C | TYR | 406 | 18.238 | 26.911 | 8.742 | 1.00 | 0.00 | 3A4 |
| ATOM | 2885 | O | TYR | 406 | 19.185 | 27.556 | 8.293 | 1.00 | 0.00 | 3A4 |
| ATOM | 2886 | N | TRP | 407 | 17.542 | 26.030 | 7.986 | 1.00 | 0.00 | 3A4 |
| ATOM | 2887 | CA | TRP | 407 | 17.869 | 25.728 | 6.609 | 1.00 | 0.00 | 3A4 |
| ATOM | 2888 | CB | TRP | 407 | 18.602 | 24.364 | 6.460 | 1.00 | 0.00 | 3A4 |
| ATOM | 2889 | CG | TRP | 407 | 19.890 | 24.534 | 5.666 | 1.00 | 0.00 | 3A4 |
| ATOM | 2890 | CD2 | TRP | 407 | 20.082 | 24.159 | 4.293 | 1.00 | 0.00 | 3A4 |
| ATOM | 2891 | CD1 | TRP | 407 | 21.050 | 25.141 | 6.063 | 1.00 | 0.00 | 3A4 |
| ATOM | 2892 | NE1 | TRP | 407 | 21.981 | 25.110 | 5.052 | 1.00 | 0.00 | 3A4 |
| ATOM | 2893 | CE2 | TRP | 407 | 21.405 | 24.521 | 3.948 | 1.00 | 0.00 | 3A4 |
| ATOM | 2894 | CE3 | TRP | 407 | 19.231 | 23.566 | 3.365 | 1.00 | 0.00 | 3A4 |
| ATOM | 2895 | CZ2 | TRP | 407 | 21.902 | 24.280 | 2.673 | 1.00 | 0.00 | 3A4 |
| ATOM | 2896 | CZ3 | TRP | 407 | 19.728 | 23.326 | 2.076 | 1.00 | 0.00 | 3A4 |
| ATOM | 2897 | CH2 | TRP | 407 | 21.046 | 23.678 | 1.741 | 1.00 | 0.00 | 3A4 |
| ATOM | 2898 | C | TRP | 407 | 16.592 | 25.684 | 5.809 | 1.00 | 0.00 | 3A4 |
| ATOM | 2899 | O | TRP | 407 | 15.735 | 24.831 | 6.029 | 1.00 | 0.00 | 3A4 |
| ATOM | 2900 | N | THR | 408 | 16.454 | 26.581 | 4.791 | 1.00 | 0.00 | 3A4 |
| ATOM | 2901 | CA | THR | 408 | 15.311 | 26.691 | 3.895 | 1.00 | 0.00 | 3A4 |
| ATOM | 2902 | CB | THR | 408 | 15.211 | 28.071 | 3.263 | 1.00 | 0.00 | 3A4 |
| ATOM | 2903 | OG1 | THR | 408 | 16.432 | 28.505 | 2.662 | 1.00 | 0.00 | 3A4 |
| ATOM | 2904 | CG2 | THR | 408 | 14.818 | 29.070 | 4.376 | 1.00 | 0.00 | 3A4 |
| ATOM | 2905 | C | THR | 408 | 15.392 | 25.598 | 2.839 | 1.00 | 0.00 | 3A4 |
| ATOM | 2906 | O | THR | 408 | 16.472 | 25.245 | 2.376 | 1.00 | 0.00 | 3A4 |
| ATOM | 2907 | N | GLU | 409 | 14.235 | 24.987 | 2.499 | 1.00 | 0.00 | 3A4 |
| ATOM | 2908 | CA | GLU | 409 | 14.103 | 23.803 | 1.664 | 1.00 | 0.00 | 3A4 |
| ATOM | 2909 | CB | GLU | 409 | 14.330 | 24.118 | 0.167 | 1.00 | 0.00 | 3A4 |
| ATOM | 2910 | CG | GLU | 409 | 13.370 | 25.190 | −0.379 | 1.00 | 0.00 | 3A4 |
| ATOM | 2911 | CD | GLU | 409 | 13.625 | 25.385 | −1.878 | 1.00 | 0.00 | 3A4 |
| ATOM | 2912 | OE1 | GLU | 409 | 12.698 | 25.099 | −2.683 | 1.00 | 0.00 | 3A4 |
| ATOM | 2913 | OE2 | GLU | 409 | 14.752 | 25.821 | −2.236 | 1.00 | 0.00 | 3A4 |
| ATOM | 2914 | C | GLU | 409 | 15.001 | 22.657 | 2.097 | 1.00 | 0.00 | 3A4 |
| ATOM | 2915 | O | GLU | 409 | 15.858 | 22.218 | 1.334 | 1.00 | 0.00 | 3A4 |
| ATOM | 2916 | N | PRO | 410 | 14.886 | 22.233 | 3.369 | 1.00 | 0.00 | 3A4 |
| ATOM | 2917 | CA | PRO | 410 | 15.857 | 21.388 | 4.073 | 1.00 | 0.00 | 3A4 |
| ATOM | 2918 | CD | PRO | 410 | 13.663 | 22.414 | 4.165 | 1.00 | 0.00 | 3A4 |
| ATOM | 2919 | CB | PRO | 410 | 15.298 | 21.307 | 5.504 | 1.00 | 0.00 | 3A4 |
| ATOM | 2920 | CG | PRO | 410 | 13.783 | 21.467 | 5.357 | 1.00 | 0.00 | 3A4 |
| ATOM | 2921 | C | PRO | 410 | 16.013 | 20.007 | 3.463 | 1.00 | 0.00 | 3A4 |
| ATOM | 2922 | O | PRO | 410 | 17.048 | 19.368 | 3.620 | 1.00 | 0.00 | 3A4 |
| ATOM | 2923 | N | GLU | 411 | 15.001 | 19.530 | 2.723 | 1.00 | 0.00 | 3A4 |
| ATOM | 2924 | CA | GLU | 411 | 15.007 | 18.249 | 2.086 | 1.00 | 0.00 | 3A4 |
| ATOM | 2925 | CB | GLU | 411 | 13.575 | 17.671 | 2.041 | 1.00 | 0.00 | 3A4 |
| ATOM | 2926 | CG | GLU | 411 | 12.398 | 18.662 | 1.868 | 1.00 | 0.00 | 3A4 |
| ATOM | 2927 | CD | GLU | 411 | 12.395 | 19.342 | 0.498 | 1.00 | 0.00 | 3A4 |
| ATOM | 2928 | OE1 | GLU | 411 | 12.459 | 20.600 | 0.464 | 1.00 | 0.00 | 3A4 |
| ATOM | 2929 | OE2 | GLU | 411 | 12.316 | 18.616 | −0.530 | 1.00 | 0.00 | 3A4 |
| ATOM | 2930 | C | GLU | 411 | 15.637 | 18.272 | 0.712 | 1.00 | 0.00 | 3A4 |
| ATOM | 2931 | O | GLU | 411 | 15.745 | 17.227 | 0.076 | 1.00 | 0.00 | 3A4 |
| ATOM | 2932 | N | LYS | 412 | 16.093 | 19.439 | 0.214 | 1.00 | 0.00 | 3A4 |
| ATOM | 2933 | CA | LYS | 412 | 16.747 | 19.560 | −1.069 | 1.00 | 0.00 | 3A4 |
| ATOM | 2934 | CB | LYS | 412 | 16.319 | 20.855 | −1.798 | 1.00 | 0.00 | 3A4 |
| ATOM | 2935 | CG | LYS | 412 | 14.842 | 20.918 | −2.228 | 1.00 | 0.00 | 3A4 |
| ATOM | 2936 | CD | LYS | 412 | 14.489 | 20.264 | −3.577 | 1.00 | 0.00 | 3A4 |
| ATOM | 2937 | CE | LYS | 412 | 14.483 | 18.726 | −3.601 | 1.00 | 0.00 | 3A4 |
| ATOM | 2938 | NZ | LYS | 412 | 13.990 | 18.220 | −4.904 | 1.00 | 0.00 | 3A4 |
| ATOM | 2939 | C | LYS | 412 | 18.246 | 19.556 | −0.876 | 1.00 | 0.00 | 3A4 |
| ATOM | 2940 | O | LYS | 412 | 18.788 | 20.180 | 0.031 | 1.00 | 0.00 | 3A4 |
| ATOM | 2941 | N | PHE | 413 | 18.970 | 18.849 | −1.771 | 1.00 | 0.00 | 3A4 |
| ATOM | 2942 | CA | PHE | 413 | 20.413 | 18.790 | −1.784 | 1.00 | 0.00 | 3A4 |
| ATOM | 2943 | CB | PHE | 413 | 20.897 | 17.450 | −2.401 | 1.00 | 0.00 | 3A4 |
| ATOM | 2944 | CG | PHE | 413 | 22.398 | 17.294 | −2.423 | 1.00 | 0.00 | 3A4 |
| ATOM | 2945 | CD1 | PHE | 413 | 23.168 | 17.227 | −1.254 | 1.00 | 0.00 | 3A4 |
| ATOM | 2946 | CD2 | PHE | 413 | 23.046 | 17.095 | −3.661 | 1.00 | 0.00 | 3A4 |
| ATOM | 2947 | CE1 | PHE | 413 | 24.550 | 16.999 | −1.315 | 1.00 | 0.00 | 3A4 |
| ATOM | 2948 | CE2 | PHE | 413 | 24.421 | 16.834 | −3.726 | 1.00 | 0.00 | 3A4 |
| ATOM | 2949 | CZ | PHE | 413 | 25.177 | 16.794 | −2.550 | 1.00 | 0.00 | 3A4 |
| ATOM | 2950 | C | PHE | 413 | 20.891 | 19.968 | −2.586 | 1.00 | 0.00 | 3A4 |
| ATOM | 2951 | O | PHE | 413 | 20.724 | 20.009 | −3.801 | 1.00 | 0.00 | 3A4 |
| ATOM | 2952 | N | LEU | 414 | 21.483 | 20.957 | −1.901 | 1.00 | 0.00 | 3A4 |
| ATOM | 2953 | CA | LEU | 414 | 21.933 | 22.177 | −2.522 | 1.00 | 0.00 | 3A4 |
| ATOM | 2954 | CB | LEU | 414 | 20.902 | 23.301 | −2.306 | 1.00 | 0.00 | 3A4 |
| ATOM | 2955 | CG | LEU | 414 | 21.169 | 24.700 | −2.902 | 1.00 | 0.00 | 3A4 |
| ATOM | 2956 | CD1 | LEU | 414 | 21.261 | 24.685 | −4.441 | 1.00 | 0.00 | 3A4 |
| ATOM | 2957 | CD2 | LEU | 414 | 20.079 | 25.663 | −2.388 | 1.00 | 0.00 | 3A4 |
| ATOM | 2958 | C | LEU | 414 | 23.275 | 22.553 | −1.966 | 1.00 | 0.00 | 3A4 |
| ATOM | 2959 | O | LEU | 414 | 23.343 | 23.031 | −0.834 | 1.00 | 0.00 | 3A4 |
| ATOM | 2960 | N | PRO | 415 | 24.394 | 22.396 | −2.681 | 1.00 | 0.00 | 3A4 |
| ATOM | 2961 | CA | PRO | 415 | 25.721 | 22.710 | −2.165 | 1.00 | 0.00 | 3A4 |
| ATOM | 2962 | CD | PRO | 415 | 24.457 | 21.932 | −4.069 | 1.00 | 0.00 | 3A4 |
| ATOM | 2963 | CB | PRO | 415 | 26.676 | 22.158 | −3.227 | 1.00 | 0.00 | 3A4 |
| ATOM | 2964 | CG | PRO | 415 | 25.882 | 22.244 | −4.535 | 1.00 | 0.00 | 3A4 |
| ATOM | 2965 | C | PRO | 415 | 25.978 | 24.200 | −1.983 | 1.00 | 0.00 | 3A4 |
| ATOM | 2966 | O | PRO | 415 | 26.655 | 24.548 | −1.013 | 1.00 | 0.00 | 3A4 |
| ATOM | 2967 | N | GLU | 416 | 25.547 | 25.077 | −2.937 | 1.00 | 0.00 | 3A4 |
| ATOM | 2968 | CA | GLU | 416 | 25.810 | 26.505 | −2.951 | 1.00 | 0.00 | 3A4 |
| ATOM | 2969 | CB | GLU | 416 | 27.244 | 26.943 | −3.438 | 1.00 | 0.00 | 3A4 |
| ATOM | 2970 | CG | GLU | 416 | 28.338 | 26.841 | −2.361 | 1.00 | 0.00 | 3A4 |
| ATOM | 2971 | CD | GLU | 416 | 29.636 | 27.541 | −2.784 | 1.00 | 0.00 | 3A4 |
| ATOM | 2972 | OE1 | GLU | 416 | 29.584 | 28.756 | −3.114 | 1.00 | 0.00 | 3A4 |
| ATOM | 2973 | OE2 | GLU | 416 | 30.704 | 26.872 | −2.759 | 1.00 | 0.00 | 3A4 |
| ATOM | 2974 | C | GLU | 416 | 24.818 | 27.126 | −3.902 | 1.00 | 0.00 | 3A4 |
| ATOM | 2975 | O | GLU | 416 | 24.357 | 26.500 | −4.855 | 1.00 | 0.00 | 3A4 |
| ATOM | 2976 | N | ARG | 417 | 24.555 | 28.429 | −3.633 | 1.00 | 0.00 | 3A4 |
| ATOM | 2977 | CA | ARG | 417 | 23.940 | 29.448 | −4.465 | 1.00 | 0.00 | 3A4 |
| ATOM | 2978 | CB | ARG | 417 | 22.623 | 29.089 | −5.229 | 1.00 | 0.00 | 3A4 |
| ATOM | 2979 | CG | ARG | 417 | 21.420 | 28.657 | −4.370 | 1.00 | 0.00 | 3A4 |
| ATOM | 2980 | CD | ARG | 417 | 20.160 | 28.317 | −5.191 | 1.00 | 0.00 | 3A4 |
| ATOM | 2981 | NE | ARG | 417 | 19.654 | 29.550 | −5.895 | 1.00 | 0.00 | 3A4 |
| ATOM | 2982 | CZ | ARG | 417 | 18.836 | 30.485 | −5.308 | 1.00 | 0.00 | 3A4 |
| ATOM | 2983 | NH1 | ARG | 417 | 18.453 | 31.575 | −6.033 | 1.00 | 0.00 | 3A4 |
| ATOM | 2984 | NH2 | ARG | 417 | 18.396 | 30.357 | −4.021 | 1.00 | 0.00 | 3A4 |
| ATOM | 2985 | C | ARG | 417 | 23.675 | 30.614 | −3.534 | 1.00 | 0.00 | 3A4 |
| ATOM | 2986 | O | ARG | 417 | 23.277 | 31.695 | −3.966 | 1.00 | 0.00 | 3A4 |
| ATOM | 2987 | N | PHE | 418 | 23.883 | 30.380 | −2.207 | 1.00 | 0.00 | 3A4 |
| ATOM | 2988 | CA | PHE | 418 | 23.598 | 31.253 | −1.087 | 1.00 | 0.00 | 3A4 |
| ATOM | 2989 | CB | PHE | 418 | 22.842 | 30.493 | 0.064 | 1.00 | 0.00 | 3A4 |
| ATOM | 2990 | CG | PHE | 418 | 23.400 | 29.109 | 0.354 | 1.00 | 0.00 | 3A4 |
| ATOM | 2991 | CD1 | PHE | 418 | 24.479 | 28.931 | 1.245 | 1.00 | 0.00 | 3A4 |
| ATOM | 2992 | CD2 | PHE | 418 | 22.845 | 27.967 | −0.262 | 1.00 | 0.00 | 3A4 |
| ATOM | 2993 | CE1 | PHE | 418 | 25.002 | 27.653 | 1.496 | 1.00 | 0.00 | 3A4 |
| ATOM | 2994 | CE2 | PHE | 418 | 23.361 | 26.688 | −0.007 | 1.00 | 0.00 | 3A4 |
| ATOM | 2995 | CZ | PHE | 418 | 24.442 | 26.532 | 0.869 | 1.00 | 0.00 | 3A4 |
| ATOM | 2996 | C | PHE | 418 | 24.891 | 31.865 | −0.589 | 1.00 | 0.00 | 3A4 |
| ATOM | 2997 | O | PHE | 418 | 25.978 | 31.337 | −0.822 | 1.00 | 0.00 | 3A4 |
| ATOM | 2998 | N | SER | 419 | 24.764 | 33.010 | 0.130 | 1.00 | 0.00 | 3A4 |
| ATOM | 2999 | CA | SER | 419 | 25.850 | 33.749 | 0.742 | 1.00 | 0.00 | 3A4 |
| ATOM | 3000 | CB | SER | 419 | 26.168 | 35.084 | 0.003 | 1.00 | 0.00 | 3A4 |
| ATOM | 3001 | OG | SER | 419 | 26.610 | 34.824 | −1.323 | 1.00 | 0.00 | 3A4 |
| ATOM | 3002 | C | SER | 419 | 25.419 | 34.042 | 2.158 | 1.00 | 0.00 | 3A4 |
| ATOM | 3003 | O | SER | 419 | 24.254 | 33.869 | 2.516 | 1.00 | 0.00 | 3A4 |
| ATOM | 3004 | N | LYS | 420 | 26.380 | 34.507 | 2.995 | 1.00 | 0.00 | 3A4 |
| ATOM | 3005 | CA | LYS | 420 | 26.162 | 34.853 | 4.383 | 1.00 | 0.00 | 3A4 |
| ATOM | 3006 | CB | LYS | 420 | 26.449 | 33.660 | 5.338 | 1.00 | 0.00 | 3A4 |
| ATOM | 3007 | CG | LYS | 420 | 26.041 | 33.882 | 6.806 | 1.00 | 0.00 | 3A4 |
| ATOM | 3008 | CD | LYS | 420 | 25.973 | 32.611 | 7.673 | 1.00 | 0.00 | 3A4 |
| ATOM | 3009 | CE | LYS | 420 | 27.316 | 31.947 | 8.025 | 1.00 | 0.00 | 3A4 |
| ATOM | 3010 | NZ | LYS | 420 | 27.897 | 31.201 | 6.884 | 1.00 | 0.00 | 3A4 |
| ATOM | 3011 | C | LYS | 420 | 27.070 | 36.024 | 4.656 | 1.00 | 0.00 | 3A4 |
| ATOM | 3012 | O | LYS | 420 | 28.205 | 36.070 | 4.182 | 1.00 | 0.00 | 3A4 |
| ATOM | 3013 | N | LYS | 421 | 26.554 | 37.018 | 5.432 | 1.00 | 0.00 | 3A4 |
| ATOM | 3014 | CA | LYS | 421 | 27.150 | 38.319 | 5.697 | 1.00 | 0.00 | 3A4 |
| ATOM | 3015 | CB | LYS | 421 | 26.041 | 39.410 | 5.739 | 1.00 | 0.00 | 3A4 |
| ATOM | 3016 | CG | LYS | 421 | 26.518 | 40.872 | 5.751 | 1.00 | 0.00 | 3A4 |
| ATOM | 3017 | CD | LYS | 421 | 25.360 | 41.868 | 5.629 | 1.00 | 0.00 | 3A4 |
| ATOM | 3018 | CE | LYS | 421 | 25.821 | 43.332 | 5.619 | 1.00 | 0.00 | 3A4 |
| ATOM | 3019 | NZ | LYS | 421 | 24.664 | 44.251 | 5.492 | 1.00 | 0.00 | 3A4 |
| ATOM | 3020 | C | LYS | 421 | 27.940 | 38.313 | 6.992 | 1.00 | 0.00 | 3A4 |
| ATOM | 3021 | O | LYS | 421 | 28.871 | 39.099 | 7.165 | 1.00 | 0.00 | 3A4 |
| ATOM | 3022 | N | ASN | 422 | 27.572 | 37.398 | 7.933 | 1.00 | 0.00 | 3A4 |
| ATOM | 3023 | CA | ASN | 422 | 28.154 | 37.237 | 9.255 | 1.00 | 0.00 | 3A4 |
| ATOM | 3024 | CB | ASN | 422 | 27.020 | 37.047 | 10.319 | 1.00 | 0.00 | 3A4 |
| ATOM | 3025 | CG | ASN | 422 | 27.499 | 37.210 | 11.776 | 1.00 | 0.00 | 3A4 |
| ATOM | 3026 | OD1 | ASN | 422 | 27.456 | 36.260 | 12.556 | 1.00 | 0.00 | 3A4 |
| ATOM | 3027 | ND2 | ASN | 422 | 27.964 | 38.434 | 12.149 | 1.00 | 0.00 | 3A4 |
| ATOM | 3028 | C | ASN | 422 | 29.107 | 36.052 | 9.222 | 1.00 | 0.00 | 3A4 |
| ATOM | 3029 | O | ASN | 422 | 29.035 | 35.203 | 8.335 | 1.00 | 0.00 | 3A4 |
| ATOM | 3030 | N | LYS | 423 | 30.033 | 35.989 | 10.220 | 1.00 | 0.00 | 3A4 |
| ATOM | 3031 | CA | LYS | 423 | 31.052 | 34.968 | 10.382 | 1.00 | 0.00 | 3A4 |
| ATOM | 3032 | CB | LYS | 423 | 32.450 | 35.576 | 10.704 | 1.00 | 0.00 | 3A4 |
| ATOM | 3033 | CG | LYS | 423 | 33.030 | 36.450 | 9.575 | 1.00 | 0.00 | 3A4 |
| ATOM | 3034 | CD | LYS | 423 | 33.266 | 35.769 | 8.209 | 1.00 | 0.00 | 3A4 |
| ATOM | 3035 | CE | LYS | 423 | 34.431 | 34.762 | 8.142 | 1.00 | 0.00 | 3A4 |
| ATOM | 3036 | NZ | LYS | 423 | 34.107 | 33.470 | 8.794 | 1.00 | 0.00 | 3A4 |
| ATOM | 3037 | C | LYS | 423 | 30.631 | 34.032 | 11.491 | 1.00 | 0.00 | 3A4 |
| ATOM | 3038 | O | LYS | 423 | 30.939 | 34.245 | 12.663 | 1.00 | 0.00 | 3A4 |
| ATOM | 3039 | N | ASP | 424 | 29.907 | 32.953 | 11.101 | 1.00 | 0.00 | 3A4 |
| ATOM | 3040 | CA | ASP | 424 | 29.457 | 31.893 | 11.976 | 1.00 | 0.00 | 3A4 |
| ATOM | 3041 | CB | ASP | 424 | 28.060 | 32.147 | 12.651 | 1.00 | 0.00 | 3A4 |
| ATOM | 3042 | CG | ASP | 424 | 26.909 | 32.519 | 11.687 | 1.00 | 0.00 | 3A4 |
| ATOM | 3043 | OD1 | ASP | 424 | 25.944 | 31.714 | 11.589 | 1.00 | 0.00 | 3A4 |
| ATOM | 3044 | OD2 | ASP | 424 | 26.973 | 33.604 | 11.051 | 1.00 | 0.00 | 3A4 |
| ATOM | 3045 | C | ASP | 424 | 29.495 | 30.648 | 11.123 | 1.00 | 0.00 | 3A4 |
| ATOM | 3046 | O | ASP | 424 | 28.470 | 30.043 | 10.811 | 1.00 | 0.00 | 3A4 |
| ATOM | 3047 | N | ASN | 425 | 30.728 | 30.251 | 10.724 | 1.00 | 0.00 | 3A4 |
| ATOM | 3048 | CA | ASN | 425 | 30.999 | 29.112 | 9.872 | 1.00 | 0.00 | 3A4 |
| ATOM | 3049 | CB | ASN | 425 | 30.910 | 29.449 | 8.339 | 1.00 | 0.00 | 3A4 |
| ATOM | 3050 | CG | ASN | 425 | 31.718 | 30.701 | 7.912 | 1.00 | 0.00 | 3A4 |
| ATOM | 3051 | OD1 | ASN | 425 | 32.894 | 30.588 | 7.570 | 1.00 | 0.00 | 3A4 |
| ATOM | 3052 | ND2 | ASN | 425 | 31.085 | 31.907 | 7.916 | 1.00 | 0.00 | 3A4 |
| ATOM | 3053 | C | ASN | 425 | 32.363 | 28.587 | 10.270 | 1.00 | 0.00 | 3A4 |
| ATOM | 3054 | O | ASN | 425 | 33.092 | 28.033 | 9.449 | 1.00 | 0.00 | 3A4 |
| ATOM | 3055 | N | ILE | 426 | 32.734 | 28.773 | 11.570 | 1.00 | 0.00 | 3A4 |
| ATOM | 3056 | CA | ILE | 426 | 34.043 | 28.493 | 12.137 | 1.00 | 0.00 | 3A4 |
| ATOM | 3057 | CB | ILE | 426 | 34.648 | 29.716 | 12.854 | 1.00 | 0.00 | 3A4 |
| ATOM | 3058 | CG2 | ILE | 426 | 36.105 | 29.402 | 13.297 | 1.00 | 0.00 | 3A4 |
| ATOM | 3059 | CG1 | ILE | 426 | 34.591 | 30.966 | 11.928 | 1.00 | 0.00 | 3A4 |
| ATOM | 3060 | CD | ILE | 426 | 35.100 | 32.260 | 12.569 | 1.00 | 0.00 | 3A4 |
| ATOM | 3061 | C | ILE | 426 | 33.870 | 27.289 | 13.047 | 1.00 | 0.00 | 3A4 |
| ATOM | 3062 | O | ILE | 426 | 34.107 | 26.155 | 12.633 | 1.00 | 0.00 | 3A4 |
| ATOM | 3063 | N | ASP | 427 | 33.447 | 27.532 | 14.315 | 1.00 | 0.00 | 3A4 |
| ATOM | 3064 | CA | ASP | 427 | 33.204 | 26.537 | 15.348 | 1.00 | 0.00 | 3A4 |
| ATOM | 3065 | CB | ASP | 427 | 34.320 | 26.450 | 16.444 | 1.00 | 0.00 | 3A4 |
| ATOM | 3066 | CG | ASP | 427 | 35.639 | 25.963 | 15.828 | 1.00 | 0.00 | 3A4 |
| ATOM | 3067 | OD1 | ASP | 427 | 35.660 | 24.821 | 15.293 | 1.00 | 0.00 | 3A4 |
| ATOM | 3068 | OD2 | ASP | 427 | 36.643 | 26.722 | 15.888 | 1.00 | 0.00 | 3A4 |
| ATOM | 3069 | C | ASP | 427 | 31.841 | 26.813 | 15.973 | 1.00 | 0.00 | 3A4 |
| ATOM | 3070 | O | ASP | 427 | 31.098 | 25.842 | 16.103 | 1.00 | 0.00 | 3A4 |
| ATOM | 3071 | N | PRO | 428 | 31.390 | 28.043 | 16.369 | 1.00 | 0.00 | 3A4 |
| ATOM | 3072 | CA | PRO | 428 | 29.983 | 28.361 | 16.637 | 1.00 | 0.00 | 3A4 |
| ATOM | 3073 | CD | PRO | 428 | 32.272 | 29.141 | 16.778 | 1.00 | 0.00 | 3A4 |
| ATOM | 3074 | CB | PRO | 428 | 30.041 | 29.613 | 17.539 | 1.00 | 0.00 | 3A4 |
| ATOM | 3075 | CG | PRO | 428 | 31.359 | 30.308 | 17.172 | 1.00 | 0.00 | 3A4 |
| ATOM | 3076 | C | PRO | 428 | 29.245 | 28.611 | 15.321 | 1.00 | 0.00 | 3A4 |
| ATOM | 3077 | O | PRO | 428 | 29.471 | 29.640 | 14.687 | 1.00 | 0.00 | 3A4 |
| ATOM | 3078 | N | TYR | 429 | 28.385 | 27.650 | 14.899 | 1.00 | 0.00 | 3A4 |
| ATOM | 3079 | CA | TYR | 429 | 27.755 | 27.649 | 13.596 | 1.00 | 0.00 | 3A4 |
| ATOM | 3080 | CB | TYR | 429 | 28.727 | 27.191 | 12.437 | 1.00 | 0.00 | 3A4 |
| ATOM | 3081 | CG | TYR | 429 | 29.325 | 25.786 | 12.428 | 1.00 | 0.00 | 3A4 |
| ATOM | 3082 | CD1 | TYR | 429 | 29.871 | 25.334 | 11.208 | 1.00 | 0.00 | 3A4 |
| ATOM | 3083 | CD2 | TYR | 429 | 29.371 | 24.901 | 13.532 | 1.00 | 0.00 | 3A4 |
| ATOM | 3084 | CE1 | TYR | 429 | 30.418 | 24.050 | 11.078 | 1.00 | 0.00 | 3A4 |
| ATOM | 3085 | CE2 | TYR | 429 | 29.917 | 23.615 | 13.410 | 1.00 | 0.00 | 3A4 |
| ATOM | 3086 | CZ | TYR | 429 | 30.437 | 23.186 | 12.181 | 1.00 | 0.00 | 3A4 |
| ATOM | 3087 | OH | TYR | 429 | 30.972 | 21.885 | 12.055 | 1.00 | 0.00 | 3A4 |
| ATOM | 3088 | C | TYR | 429 | 26.500 | 26.805 | 13.683 | 1.00 | 0.00 | 3A4 |
| ATOM | 3089 | O | TYR | 429 | 25.984 | 26.542 | 14.769 | 1.00 | 0.00 | 3A4 |
| ATOM | 3090 | N | ILE | 430 | 26.004 | 26.339 | 12.499 | 1.00 | 0.00 | 3A4 |
| ATOM | 3091 | CA | ILE | 430 | 24.912 | 25.392 | 12.301 | 1.00 | 0.00 | 3A4 |
| ATOM | 3092 | CB | ILE | 430 | 23.953 | 25.845 | 11.196 | 1.00 | 0.00 | 3A4 |
| ATOM | 3093 | CG2 | ILE | 430 | 23.323 | 27.171 | 11.680 | 1.00 | 0.00 | 3A4 |
| ATOM | 3094 | CG1 | ILE | 430 | 24.623 | 26.004 | 9.800 | 1.00 | 0.00 | 3A4 |
| ATOM | 3095 | CD | ILE | 430 | 23.667 | 26.472 | 8.700 | 1.00 | 0.00 | 3A4 |
| ATOM | 3096 | C | ILE | 430 | 25.562 | 24.051 | 12.005 | 1.00 | 0.00 | 3A4 |
| ATOM | 3097 | O | ILE | 430 | 26.789 | 23.977 | 12.016 | 1.00 | 0.00 | 3A4 |
| ATOM | 3098 | N | TYR | 431 | 24.773 | 22.951 | 11.760 | 1.00 | 0.00 | 3A4 |
| ATOM | 3099 | CA | TYR | 431 | 25.198 | 21.604 | 11.414 | 1.00 | 0.00 | 3A4 |
| ATOM | 3100 | CB | TYR | 431 | 26.437 | 21.470 | 10.410 | 1.00 | 0.00 | 3A4 |
| ATOM | 3101 | CG | TYR | 431 | 26.384 | 22.218 | 9.097 | 1.00 | 0.00 | 3A4 |
| ATOM | 3102 | CD1 | TYR | 431 | 25.749 | 21.647 | 7.983 | 1.00 | 0.00 | 3A4 |
| ATOM | 3103 | CD2 | TYR | 431 | 27.142 | 23.393 | 8.894 | 1.00 | 0.00 | 3A4 |
| ATOM | 3104 | CE1 | TYR | 431 | 25.834 | 22.246 | 6.714 | 1.00 | 0.00 | 3A4 |
| ATOM | 3105 | CE2 | TYR | 431 | 27.212 | 24.010 | 7.638 | 1.00 | 0.00 | 3A4 |
| ATOM | 3106 | CZ | TYR | 431 | 26.551 | 23.437 | 6.544 | 1.00 | 0.00 | 3A4 |
| ATOM | 3107 | OH | TYR | 431 | 26.632 | 24.036 | 5.267 | 1.00 | 0.00 | 3A4 |
| ATOM | 3108 | C | TYR | 431 | 25.647 | 20.889 | 12.688 | 1.00 | 0.00 | 3A4 |
| ATOM | 3109 | O | TYR | 431 | 26.635 | 21.285 | 13.303 | 1.00 | 0.00 | 3A4 |
| ATOM | 3110 | N | THR | 432 | 24.989 | 19.782 | 13.089 | 1.00 | 0.00 | 3A4 |
| ATOM | 3111 | CA | THR | 432 | 25.445 | 18.960 | 14.203 | 1.00 | 0.00 | 3A4 |
| ATOM | 3112 | CB | THR | 432 | 24.381 | 18.938 | 15.316 | 1.00 | 0.00 | 3A4 |
| ATOM | 3113 | OG1 | THR | 432 | 23.070 | 18.659 | 14.826 | 1.00 | 0.00 | 3A4 |
| ATOM | 3114 | CG2 | THR | 432 | 24.399 | 20.310 | 16.028 | 1.00 | 0.00 | 3A4 |
| ATOM | 3115 | C | THR | 432 | 25.810 | 17.558 | 13.781 | 1.00 | 0.00 | 3A4 |
| ATOM | 3116 | O | THR | 432 | 25.565 | 16.648 | 14.576 | 1.00 | 0.00 | 3A4 |
| ATOM | 3117 | N | PRO | 433 | 26.389 | 17.234 | 12.610 | 1.00 | 0.00 | 3A4 |
| ATOM | 3118 | CA | PRO | 433 | 26.601 | 15.849 | 12.240 | 1.00 | 0.00 | 3A4 |
| ATOM | 3119 | CD | PRO | 433 | 27.173 | 18.101 | 11.724 | 1.00 | 0.00 | 3A4 |
| ATOM | 3120 | CB | PRO | 433 | 26.947 | 15.918 | 10.740 | 1.00 | 0.00 | 3A4 |
| ATOM | 3121 | CG | PRO | 433 | 27.743 | 17.221 | 10.601 | 1.00 | 0.00 | 3A4 |
| ATOM | 3122 | C | PRO | 433 | 27.748 | 15.209 | 13.022 | 1.00 | 0.00 | 3A4 |
| ATOM | 3123 | O | PRO | 433 | 27.771 | 13.993 | 13.150 | 1.00 | 0.00 | 3A4 |
| ATOM | 3124 | N | PHE | 434 | 28.673 | 16.013 | 13.587 | 1.00 | 0.00 | 3A4 |
| ATOM | 3125 | CA | PHE | 434 | 29.782 | 15.548 | 14.382 | 1.00 | 0.00 | 3A4 |
| ATOM | 3126 | CB | PHE | 434 | 31.108 | 16.257 | 13.997 | 1.00 | 0.00 | 3A4 |
| ATOM | 3127 | CG | PHE | 434 | 31.591 | 15.810 | 12.639 | 1.00 | 0.00 | 3A4 |
| ATOM | 3128 | CD1 | PHE | 434 | 31.353 | 16.451 | 11.514 | 1.00 | 0.00 | 3A4 |
| ATOM | 3129 | CD2 | PHE | 434 | 32.354 | 14.754 | 12.363 | 1.00 | 0.00 | 3A4 |
| ATOM | 3130 | CE1 | PHE | 434 | 31.813 | 16.203 | 10.200 | 1.00 | 0.00 | 3A4 |
| ATOM | 3131 | CE2 | PHE | 434 | 32.860 | 14.287 | 11.129 | 1.00 | 0.00 | 3A4 |
| ATOM | 3132 | CZ | PHE | 434 | 32.592 | 15.066 | 10.009 | 1.00 | 0.00 | 3A4 |
| ATOM | 3133 | C | PHE | 434 | 29.528 | 15.802 | 15.833 | 1.00 | 0.00 | 3A4 |
| ATOM | 3134 | O | PHE | 434 | 30.427 | 15.663 | 16.655 | 1.00 | 0.00 | 3A4 |
| ATOM | 3135 | N | GLY | 435 | 28.291 | 16.210 | 16.191 | 1.00 | 0.00 | 3A4 |
| ATOM | 3136 | CA | GLY | 435 | 27.923 | 16.495 | 17.561 | 1.00 | 0.00 | 3A4 |
| ATOM | 3137 | C | GLY | 435 | 28.209 | 17.917 | 17.875 | 1.00 | 0.00 | 3A4 |
| ATOM | 3138 | O | GLY | 435 | 28.305 | 18.719 | 16.943 | 1.00 | 0.00 | 3A4 |
| ATOM | 3139 | N | SER | 436 | 28.348 | 18.248 | 19.183 | 1.00 | 0.00 | 3A4 |
| ATOM | 3140 | CA | SER | 436 | 28.384 | 19.623 | 19.578 | 1.00 | 0.00 | 3A4 |
| ATOM | 3141 | CB | SER | 436 | 26.947 | 20.258 | 19.543 | 1.00 | 0.00 | 3A4 |
| ATOM | 3142 | OG | SER | 436 | 26.948 | 21.683 | 19.489 | 1.00 | 0.00 | 3A4 |
| ATOM | 3143 | C | SER | 436 | 29.197 | 19.916 | 20.769 | 1.00 | 0.00 | 3A4 |
| ATOM | 3144 | O | SER | 436 | 30.134 | 20.642 | 20.647 | 1.00 | 0.00 | 3A4 |
| ATOM | 3145 | N | GLY | 437 | 28.951 | 19.486 | 21.973 | 1.00 | 0.00 | 3A4 |
| ATOM | 3146 | CA | GLY | 437 | 29.675 | 19.961 | 23.144 | 1.00 | 0.00 | 3A4 |
| ATOM | 3147 | C | GLY | 437 | 31.130 | 19.558 | 23.314 | 1.00 | 0.00 | 3A4 |
| ATOM | 3148 | O | GLY | 437 | 31.959 | 19.487 | 22.402 | 1.00 | 0.00 | 3A4 |
| ATOM | 3149 | N | PRO | 438 | 31.502 | 19.228 | 24.529 | 1.00 | 0.00 | 3A4 |
| ATOM | 3150 | CA | PRO | 438 | 32.875 | 18.727 | 24.776 | 1.00 | 0.00 | 3A4 |
| ATOM | 3151 | CD | PRO | 438 | 30.927 | 19.742 | 25.770 | 1.00 | 0.00 | 3A4 |
| ATOM | 3152 | CB | PRO | 438 | 33.100 | 18.880 | 26.266 | 1.00 | 0.00 | 3A4 |
| ATOM | 3153 | CG | PRO | 438 | 31.706 | 19.063 | 26.880 | 1.00 | 0.00 | 3A4 |
| ATOM | 3154 | C | PRO | 438 | 33.099 | 17.295 | 24.317 | 1.00 | 0.00 | 3A4 |
| ATOM | 3155 | O | PRO | 438 | 34.230 | 16.824 | 24.348 | 1.00 | 0.00 | 3A4 |
| ATOM | 3156 | N | ARG | 439 | 32.033 | 16.608 | 23.850 | 1.00 | 0.00 | 3A4 |
| ATOM | 3157 | CA | ARG | 439 | 32.068 | 15.264 | 23.366 | 1.00 | 0.00 | 3A4 |
| ATOM | 3158 | CB | ARG | 439 | 30.905 | 14.467 | 23.961 | 1.00 | 0.00 | 3A4 |
| ATOM | 3159 | CG | ARG | 439 | 30.793 | 14.766 | 25.461 | 1.00 | 0.00 | 3A4 |
| ATOM | 3160 | CD | ARG | 439 | 30.197 | 13.690 | 26.366 | 1.00 | 0.00 | 3A4 |
| ATOM | 3161 | NE | ARG | 439 | 30.391 | 14.136 | 27.786 | 1.00 | 0.00 | 3A4 |
| ATOM | 3162 | CZ | ARG | 439 | 30.091 | 13.354 | 28.866 | 1.00 | 0.00 | 3A4 |
| ATOM | 3163 | NH1 | ARG | 439 | 30.342 | 13.836 | 30.116 | 1.00 | 0.00 | 3A4 |
| ATOM | 3164 | NH2 | ARG | 439 | 29.553 | 12.109 | 28.713 | 1.00 | 0.00 | 3A4 |
| ATOM | 3165 | C | ARG | 439 | 31.980 | 15.221 | 21.890 | 1.00 | 0.00 | 3A4 |
| ATOM | 3166 | O | ARG | 439 | 31.776 | 14.165 | 21.309 | 1.00 | 0.00 | 3A4 |
| ATOM | 3167 | N | ASN | 440 | 32.201 | 16.364 | 21.200 | 1.00 | 0.00 | 3A4 |
| ATOM | 3168 | CA | ASN | 440 | 32.204 | 16.474 | 19.756 | 1.00 | 0.00 | 3A4 |
| ATOM | 3169 | CB | ASN | 440 | 32.625 | 17.886 | 19.354 | 1.00 | 0.00 | 3A4 |
| ATOM | 3170 | CG | ASN | 440 | 32.279 | 18.329 | 17.901 | 1.00 | 0.00 | 3A4 |
| ATOM | 3171 | OD1 | ASN | 440 | 32.746 | 17.778 | 16.907 | 1.00 | 0.00 | 3A4 |
| ATOM | 3172 | ND2 | ASN | 440 | 31.452 | 19.401 | 17.765 | 1.00 | 0.00 | 3A4 |
| ATOM | 3173 | C | ASN | 440 | 33.216 | 15.539 | 19.171 | 1.00 | 0.00 | 3A4 |
| ATOM | 3174 | O | ASN | 440 | 34.241 | 15.285 | 19.807 | 1.00 | 0.00 | 3A4 |
| ATOM | 3175 | N | CYS | 441 | 32.950 | 14.993 | 17.974 | 1.00 | 0.00 | 3A4 |
| ATOM | 3176 | CA | CYS | 441 | 33.782 | 14.009 | 17.328 | 1.00 | 0.00 | 3A4 |
| ATOM | 3177 | CB | CYS | 441 | 33.288 | 13.772 | 15.899 | 1.00 | 0.00 | 3A4 |
| ATOM | 3178 | SG | CYS | 441 | 34.028 | 12.338 | 15.029 | 1.00 | 0.00 | 3A4 |
| ATOM | 3179 | C | CYS | 441 | 35.241 | 14.414 | 17.264 | 1.00 | 0.00 | 3A4 |
| ATOM | 3180 | O | CYS | 441 | 35.564 | 15.528 | 16.866 | 1.00 | 0.00 | 3A4 |
| ATOM | 3181 | N | ILE | 442 | 36.131 | 13.531 | 17.746 | 1.00 | 0.00 | 3A4 |
| ATOM | 3182 | CA | ILE | 442 | 37.556 | 13.799 | 17.790 | 1.00 | 0.00 | 3A4 |
| ATOM | 3183 | CB | ILE | 442 | 38.223 | 12.944 | 18.848 | 1.00 | 0.00 | 3A4 |
| ATOM | 3184 | CG2 | ILE | 442 | 38.388 | 11.476 | 18.448 | 1.00 | 0.00 | 3A4 |
| ATOM | 3185 | CG1 | ILE | 442 | 39.528 | 13.570 | 19.391 | 1.00 | 0.00 | 3A4 |
| ATOM | 3186 | CD | ILE | 442 | 39.336 | 14.819 | 20.250 | 1.00 | 0.00 | 3A4 |
| ATOM | 3187 | C | ILE | 442 | 38.181 | 13.605 | 16.408 | 1.00 | 0.00 | 3A4 |
| ATOM | 3188 | O | ILE | 442 | 39.180 | 14.220 | 16.048 | 1.00 | 0.00 | 3A4 |
| ATOM | 3189 | N | GLY | 443 | 37.524 | 12.758 | 15.590 | 1.00 | 0.00 | 3A4 |
| ATOM | 3190 | CA | GLY | 443 | 37.942 | 12.408 | 14.266 | 1.00 | 0.00 | 3A4 |
| ATOM | 3191 | C | GLY | 443 | 37.399 | 13.252 | 13.158 | 1.00 | 0.00 | 3A4 |
| ATOM | 3192 | O | GLY | 443 | 37.591 | 12.902 | 12.001 | 1.00 | 0.00 | 3A4 |
| ATOM | 3193 | N | MET | 444 | 36.732 | 14.396 | 13.456 | 1.00 | 0.00 | 3A4 |
| ATOM | 3194 | CA | MET | 444 | 36.082 | 15.266 | 12.493 | 1.00 | 0.00 | 3A4 |
| ATOM | 3195 | CB | MET | 444 | 35.408 | 16.458 | 13.192 | 1.00 | 0.00 | 3A4 |
| ATOM | 3196 | CG | MET | 444 | 36.294 | 17.310 | 14.126 | 1.00 | 0.00 | 3A4 |
| ATOM | 3197 | SD | MET | 444 | 35.375 | 18.583 | 15.041 | 1.00 | 0.00 | 3A4 |
| ATOM | 3198 | CE | MET | 444 | 36.695 | 18.911 | 16.244 | 1.00 | 0.00 | 3A4 |
| ATOM | 3199 | C | MET | 444 | 36.968 | 15.807 | 11.395 | 1.00 | 0.00 | 3A4 |
| ATOM | 3200 | O | MET | 444 | 36.570 | 15.844 | 10.236 | 1.00 | 0.00 | 3A4 |
| ATOM | 3201 | N | ARG | 445 | 38.230 | 16.170 | 11.727 | 1.00 | 0.00 | 3A4 |
| ATOM | 3202 | CA | ARG | 445 | 39.211 | 16.708 | 10.801 | 1.00 | 0.00 | 3A4 |
| ATOM | 3203 | CB | ARG | 445 | 40.435 | 17.321 | 11.549 | 1.00 | 0.00 | 3A4 |
| ATOM | 3204 | CG | ARG | 445 | 40.058 | 18.332 | 12.647 | 1.00 | 0.00 | 3A4 |
| ATOM | 3205 | CD | ARG | 445 | 39.223 | 19.529 | 12.155 | 1.00 | 0.00 | 3A4 |
| ATOM | 3206 | NE | ARG | 445 | 38.880 | 20.389 | 13.341 | 1.00 | 0.00 | 3A4 |
| ATOM | 3207 | CZ | ARG | 445 | 37.901 | 21.351 | 13.313 | 1.00 | 0.00 | 3A4 |
| ATOM | 3208 | NH1 | ARG | 445 | 37.643 | 22.071 | 14.443 | 1.00 | 0.00 | 3A4 |
| ATOM | 3209 | NH2 | ARG | 445 | 37.171 | 21.600 | 12.186 | 1.00 | 0.00 | 3A4 |
| ATOM | 3210 | C | ARG | 445 | 39.695 | 15.651 | 9.828 | 1.00 | 0.00 | 3A4 |
| ATOM | 3211 | O | ARG | 445 | 39.790 | 15.886 | 8.628 | 1.00 | 0.00 | 3A4 |
| ATOM | 3212 | N | PHE | 446 | 39.920 | 14.418 | 10.335 | 1.00 | 0.00 | 3A4 |
| ATOM | 3213 | CA | PHE | 446 | 40.293 | 13.256 | 9.549 | 1.00 | 0.00 | 3A4 |
| ATOM | 3214 | CB | PHE | 446 | 40.683 | 12.116 | 10.501 | 1.00 | 0.00 | 3A4 |
| ATOM | 3215 | CG | PHE | 446 | 41.549 | 11.008 | 9.982 | 1.00 | 0.00 | 3A4 |
| ATOM | 3216 | CD1 | PHE | 446 | 42.800 | 11.292 | 9.403 | 1.00 | 0.00 | 3A4 |
| ATOM | 3217 | CD2 | PHE | 446 | 41.248 | 9.684 | 10.350 | 1.00 | 0.00 | 3A4 |
| ATOM | 3218 | CE1 | PHE | 446 | 43.749 | 10.278 | 9.241 | 1.00 | 0.00 | 3A4 |
| ATOM | 3219 | CE2 | PHE | 446 | 42.210 | 8.678 | 10.222 | 1.00 | 0.00 | 3A4 |
| ATOM | 3220 | CZ | PHE | 446 | 43.469 | 8.983 | 9.689 | 1.00 | 0.00 | 3A4 |
| ATOM | 3221 | C | PHE | 446 | 39.172 | 12.804 | 8.647 | 1.00 | 0.00 | 3A4 |
| ATOM | 3222 | O | PHE | 446 | 39.384 | 12.508 | 7.480 | 1.00 | 0.00 | 3A4 |
| ATOM | 3223 | N | ALA | 447 | 37.921 | 12.812 | 9.148 | 1.00 | 0.00 | 3A4 |
| ATOM | 3224 | CA | ALA | 447 | 36.738 | 12.472 | 8.393 | 1.00 | 0.00 | 3A4 |
| ATOM | 3225 | CB | ALA | 447 | 35.499 | 12.464 | 9.272 | 1.00 | 0.00 | 3A4 |
| ATOM | 3226 | C | ALA | 447 | 36.489 | 13.392 | 7.228 | 1.00 | 0.00 | 3A4 |
| ATOM | 3227 | O | ALA | 447 | 36.216 | 12.950 | 6.120 | 1.00 | 0.00 | 3A4 |
| ATOM | 3228 | N | LEU | 448 | 36.656 | 14.715 | 7.427 | 1.00 | 0.00 | 3A4 |
| ATOM | 3229 | CA | LEU | 448 | 36.469 | 15.704 | 6.386 | 1.00 | 0.00 | 3A4 |
| ATOM | 3230 | CB | LEU | 448 | 36.399 | 17.124 | 6.962 | 1.00 | 0.00 | 3A4 |
| ATOM | 3231 | CG | LEU | 448 | 35.089 | 17.408 | 7.731 | 1.00 | 0.00 | 3A4 |
| ATOM | 3232 | CD1 | LEU | 448 | 35.223 | 18.721 | 8.515 | 1.00 | 0.00 | 3A4 |
| ATOM | 3233 | CD2 | LEU | 448 | 33.842 | 17.445 | 6.826 | 1.00 | 0.00 | 3A4 |
| ATOM | 3234 | C | LEU | 448 | 37.561 | 15.661 | 5.351 | 1.00 | 0.00 | 3A4 |
| ATOM | 3235 | O | LEU | 448 | 37.299 | 15.865 | 4.175 | 1.00 | 0.00 | 3A4 |
| ATOM | 3236 | N | MET | 449 | 38.806 | 15.319 | 5.746 | 1.00 | 0.00 | 3A4 |
| ATOM | 3237 | CA | MET | 449 | 39.935 | 15.178 | 4.853 | 1.00 | 0.00 | 3A4 |
| ATOM | 3238 | CB | MET | 449 | 41.269 | 15.033 | 5.617 | 1.00 | 0.00 | 3A4 |
| ATOM | 3239 | CG | MET | 449 | 41.841 | 16.358 | 6.130 | 1.00 | 0.00 | 3A4 |
| ATOM | 3240 | SD | MET | 449 | 43.382 | 16.135 | 7.075 | 1.00 | 0.00 | 3A4 |
| ATOM | 3241 | CE | MET | 449 | 43.701 | 17.904 | 7.332 | 1.00 | 0.00 | 3A4 |
| ATOM | 3242 | C | MET | 449 | 39.772 | 13.984 | 3.951 | 1.00 | 0.00 | 3A4 |
| ATOM | 3243 | O | MET | 449 | 39.956 | 14.095 | 2.746 | 1.00 | 0.00 | 3A4 |
| ATOM | 3244 | N | ASN | 450 | 39.332 | 12.829 | 4.502 | 1.00 | 0.00 | 3A4 |
| ATOM | 3245 | CA | ASN | 450 | 39.098 | 11.625 | 3.740 | 1.00 | 0.00 | 3A4 |
| ATOM | 3246 | CB | ASN | 450 | 38.915 | 10.385 | 4.623 | 1.00 | 0.00 | 3A4 |
| ATOM | 3247 | CG | ASN | 450 | 37.685 | 10.130 | 5.531 | 1.00 | 0.00 | 3A4 |
| ATOM | 3248 | OD1 | ASN | 450 | 36.512 | 10.268 | 5.191 | 1.00 | 0.00 | 3A4 |
| ATOM | 3249 | ND2 | ASN | 450 | 37.990 | 9.606 | 6.751 | 1.00 | 0.00 | 3A4 |
| ATOM | 3250 | C | ASN | 450 | 37.976 | 11.740 | 2.750 | 1.00 | 0.00 | 3A4 |
| ATOM | 3251 | O | ASN | 450 | 38.095 | 11.281 | 1.623 | 1.00 | 0.00 | 3A4 |
| ATOM | 3252 | N | MET | 451 | 36.874 | 12.422 | 3.129 | 1.00 | 0.00 | 3A4 |
| ATOM | 3253 | CA | MET | 451 | 35.744 | 12.687 | 2.267 | 1.00 | 0.00 | 3A4 |
| ATOM | 3254 | CB | MET | 451 | 34.596 | 13.445 | 3.021 | 1.00 | 0.00 | 3A4 |
| ATOM | 3255 | CG | MET | 451 | 33.788 | 12.610 | 4.010 | 1.00 | 0.00 | 3A4 |
| ATOM | 3256 | SD | MET | 451 | 32.502 | 13.565 | 4.863 | 1.00 | 0.00 | 3A4 |
| ATOM | 3257 | CE | MET | 451 | 33.061 | 13.383 | 6.570 | 1.00 | 0.00 | 3A4 |
| ATOM | 3258 | C | MET | 451 | 36.081 | 13.580 | 1.106 | 1.00 | 0.00 | 3A4 |
| ATOM | 3259 | O | MET | 451 | 35.746 | 13.314 | −0.041 | 1.00 | 0.00 | 3A4 |
| ATOM | 3260 | N | LYS | 452 | 36.821 | 14.667 | 1.389 | 1.00 | 0.00 | 3A4 |
| ATOM | 3261 | CA | LYS | 452 | 37.237 | 15.652 | 0.424 | 1.00 | 0.00 | 3A4 |
| ATOM | 3262 | CB | LYS | 452 | 37.854 | 16.860 | 1.154 | 1.00 | 0.00 | 3A4 |
| ATOM | 3263 | CG | LYS | 452 | 37.750 | 18.227 | 0.470 | 1.00 | 0.00 | 3A4 |
| ATOM | 3264 | CD | LYS | 452 | 38.346 | 19.380 | 1.301 | 1.00 | 0.00 | 3A4 |
| ATOM | 3265 | CE | LYS | 452 | 38.006 | 19.391 | 2.808 | 1.00 | 0.00 | 3A4 |
| ATOM | 3266 | NZ | LYS | 452 | 36.543 | 19.337 | 3.053 | 1.00 | 0.00 | 3A4 |
| ATOM | 3267 | C | LYS | 452 | 38.228 | 15.110 | −0.572 | 1.00 | 0.00 | 3A4 |
| ATOM | 3268 | O | LYS | 452 | 38.108 | 15.335 | −1.769 | 1.00 | 0.00 | 3A4 |
| ATOM | 3269 | N | LEU | 453 | 39.207 | 14.309 | −0.102 | 1.00 | 0.00 | 3A4 |
| ATOM | 3270 | CA | LEU | 453 | 40.212 | 13.676 | −0.927 | 1.00 | 0.00 | 3A4 |
| ATOM | 3271 | CB | LEU | 453 | 41.342 | 13.071 | −0.061 | 1.00 | 0.00 | 3A4 |
| ATOM | 3272 | CG | LEU | 453 | 42.298 | 14.219 | 0.403 | 1.00 | 0.00 | 3A4 |
| ATOM | 3273 | CD1 | LEU | 453 | 43.187 | 13.854 | 1.595 | 1.00 | 0.00 | 3A4 |
| ATOM | 3274 | CD2 | LEU | 453 | 43.191 | 14.764 | −0.734 | 1.00 | 0.00 | 3A4 |
| ATOM | 3275 | C | LEU | 453 | 39.621 | 12.644 | −1.845 | 1.00 | 0.00 | 3A4 |
| ATOM | 3276 | O | LEU | 453 | 39.939 | 12.613 | −3.026 | 1.00 | 0.00 | 3A4 |
| ATOM | 3277 | N | ALA | 454 | 38.662 | 11.829 | −1.353 | 1.00 | 0.00 | 3A4 |
| ATOM | 3278 | CA | ALA | 454 | 37.935 | 10.847 | −2.131 | 1.00 | 0.00 | 3A4 |
| ATOM | 3279 | CB | ALA | 454 | 37.013 | 9.992 | −1.240 | 1.00 | 0.00 | 3A4 |
| ATOM | 3280 | C | ALA | 454 | 37.093 | 11.464 | −3.225 | 1.00 | 0.00 | 3A4 |
| ATOM | 3281 | O | ALA | 454 | 37.181 | 11.055 | −4.371 | 1.00 | 0.00 | 3A4 |
| ATOM | 3282 | N | LEU | 455 | 36.307 | 12.514 | −2.904 | 1.00 | 0.00 | 3A4 |
| ATOM | 3283 | CA | LEU | 455 | 35.457 | 13.201 | −3.852 | 1.00 | 0.00 | 3A4 |
| ATOM | 3284 | CB | LEU | 455 | 34.480 | 14.157 | −3.157 | 1.00 | 0.00 | 3A4 |
| ATOM | 3285 | CG | LEU | 455 | 33.366 | 13.559 | −2.303 | 1.00 | 0.00 | 3A4 |
| ATOM | 3286 | CD1 | LEU | 455 | 32.576 | 14.761 | −1.760 | 1.00 | 0.00 | 3A4 |
| ATOM | 3287 | CD2 | LEU | 455 | 32.474 | 12.556 | −3.064 | 1.00 | 0.00 | 3A4 |
| ATOM | 3288 | C | LEU | 455 | 36.203 | 14.018 | −4.886 | 1.00 | 0.00 | 3A4 |
| ATOM | 3289 | O | LEU | 455 | 35.736 | 14.147 | −6.009 | 1.00 | 0.00 | 3A4 |
| ATOM | 3290 | N | ILE | 456 | 37.412 | 14.540 | −4.555 | 1.00 | 0.00 | 3A4 |
| ATOM | 3291 | CA | ILE | 456 | 38.824 | 15.264 | −5.474 | 1.00 | 0.00 | 3A4 |
| ATOM | 3292 | CB | ILE | 456 | 39.413 | 16.061 | −4.749 | 1.00 | 0.00 | 3A4 |
| ATOM | 3293 | CG2 | ILE | 456 | 40.832 | 16.074 | −5.412 | 1.00 | 0.00 | 3A4 |
| ATOM | 3294 | CG1 | ILE | 456 | 39.003 | 17.537 | −4.565 | 1.00 | 0.00 | 3A4 |
| ATOM | 3295 | CD | ILE | 456 | 38.041 | 17.823 | −3.423 | 1.00 | 0.00 | 3A4 |
| ATOM | 3296 | C | ILE | 456 | 38.894 | 14.318 | −6.496 | 1.00 | 0.00 | 3A4 |
| ATOM | 3297 | O | ILE | 456 | 39.099 | 14.677 | −7.647 | 1.00 | 0.00 | 3A4 |
| ATOM | 3298 | N | ARG | 457 | 39.181 | 13.066 | −6.083 | 1.00 | 0.00 | 3A4 |
| ATOM | 3299 | CA | ARG | 457 | 39.859 | 12.081 | −6.895 | 1.00 | 0.00 | 3A4 |
| ATOM | 3300 | CB | ARG | 457 | 40.758 | 11.186 | −6.025 | 1.00 | 0.00 | 3A4 |
| ATOM | 3301 | CG | ARG | 457 | 41.905 | 12.005 | −5.430 | 1.00 | 0.00 | 3A4 |
| ATOM | 3302 | CD | ARG | 457 | 42.735 | 11.300 | −4.341 | 1.00 | 0.00 | 3A4 |
| ATOM | 3303 | NE | ARG | 457 | 43.559 | 12.343 | −3.632 | 1.00 | 0.00 | 3A4 |
| ATOM | 3304 | CZ | ARG | 457 | 44.624 | 12.985 | −4.211 | 1.00 | 0.00 | 3A4 |
| ATOM | 3305 | NH1 | ARG | 457 | 45.148 | 14.087 | −3.601 | 1.00 | 0.00 | 3A4 |
| ATOM | 3306 | NH2 | ARG | 457 | 45.174 | 12.558 | −5.385 | 1.00 | 0.00 | 3A4 |
| ATOM | 3307 | C | ARG | 457 | 38.924 | 11.235 | −7.722 | 1.00 | 0.00 | 3A4 |
| ATOM | 3308 | O | ARG | 457 | 39.246 | 10.873 | −8.848 | 1.00 | 0.00 | 3A4 |
| ATOM | 3309 | N | VAL | 458 | 37.713 | 10.918 | −7.207 | 1.00 | 0.00 | 3A4 |
| ATOM | 3310 | CA | VAL | 458 | 36.711 | 10.092 | −7.872 | 1.00 | 0.00 | 3A4 |
| ATOM | 3311 | CB | VAL | 458 | 35.631 | 9.662 | −6.887 | 1.00 | 0.00 | 3A4 |
| ATOM | 3312 | CG1 | VAL | 458 | 34.290 | 9.133 | −7.471 | 1.00 | 0.00 | 3A4 |
| ATOM | 3313 | CG2 | VAL | 458 | 36.253 | 8.556 | −6.006 | 1.00 | 0.00 | 3A4 |
| ATOM | 3314 | C | VAL | 458 | 36.101 | 10.798 | −9.063 | 1.00 | 0.00 | 3A4 |
| ATOM | 3315 | O | VAL | 458 | 36.040 | 10.236 | −10.146 | 1.00 | 0.00 | 3A4 |
| ATOM | 3316 | N | LEU | 459 | 35.685 | 12.071 | −8.900 | 1.00 | 0.00 | 3A4 |
| ATOM | 3317 | CA | LEU | 459 | 34.979 | 12.841 | −9.906 | 1.00 | 0.00 | 3A4 |
| ATOM | 3318 | CB | LEU | 459 | 34.354 | 14.096 | −9.255 | 1.00 | 0.00 | 3A4 |
| ATOM | 3319 | CG | LEU | 459 | 33.225 | 13.778 | −8.265 | 1.00 | 0.00 | 3A4 |
| ATOM | 3320 | CD1 | LEU | 459 | 32.876 | 15.037 | −7.500 | 1.00 | 0.00 | 3A4 |
| ATOM | 3321 | CD2 | LEU | 459 | 32.041 | 13.132 | −8.991 | 1.00 | 0.00 | 3A4 |
| ATOM | 3322 | C | LEU | 459 | 35.826 | 13.309 | −11.060 | 1.00 | 0.00 | 3A4 |
| ATOM | 3323 | O | LEU | 459 | 35.319 | 13.601 | −12.136 | 1.00 | 0.00 | 3A4 |
| ATOM | 3324 | N | GLN | 460 | 37.158 | 13.371 | −10.874 | 1.00 | 0.00 | 3A4 |
| ATOM | 3325 | CA | GLN | 460 | 38.070 | 13.748 | −11.927 | 1.00 | 0.00 | 3A4 |
| ATOM | 3326 | CB | GLN | 460 | 39.373 | 14.365 | −11.376 | 1.00 | 0.00 | 3A4 |
| ATOM | 3327 | CG | GLN | 460 | 39.106 | 15.740 | −10.736 | 1.00 | 0.00 | 3A4 |
| ATOM | 3328 | CD | GLN | 460 | 40.408 | 16.286 | −10.160 | 1.00 | 0.00 | 3A4 |
| ATOM | 3329 | OE1 | GLN | 460 | 41.430 | 15.608 | −10.237 | 1.00 | 0.00 | 3A4 |
| ATOM | 3330 | NE2 | GLN | 460 | 40.380 | 17.515 | −9.575 | 1.00 | 0.00 | 3A4 |
| ATOM | 3331 | C | GLN | 460 | 38.365 | 12.562 | −12.815 | 1.00 | 0.00 | 3A4 |
| ATOM | 3332 | O | GLN | 460 | 38.683 | 12.730 | −13.990 | 1.00 | 0.00 | 3A4 |
| ATOM | 3333 | N | ASN | 461 | 38.258 | 11.332 | −12.266 | 1.00 | 0.00 | 3A4 |
| ATOM | 3334 | CA | ASN | 461 | 38.598 | 10.113 | −12.957 | 1.00 | 0.00 | 3A4 |
| ATOM | 3335 | CB | ASN | 461 | 39.328 | 9.125 | −12.018 | 1.00 | 0.00 | 3A4 |
| ATOM | 3336 | CG | ASN | 461 | 40.725 | 9.722 | −11.730 | 1.00 | 0.00 | 3A4 |
| ATOM | 3337 | OD1 | ASN | 461 | 41.364 | 10.283 | −12.617 | 1.00 | 0.00 | 3A4 |
| ATOM | 3338 | ND2 | ASN | 461 | 41.239 | 9.604 | −10.482 | 1.00 | 0.00 | 3A4 |
| ATOM | 3339 | C | ASN | 461 | 37.402 | 9.427 | −13.562 | 1.00 | 0.00 | 3A4 |
| ATOM | 3340 | O | ASN | 461 | 37.588 | 8.603 | −14.451 | 1.00 | 0.00 | 3A4 |
| ATOM | 3341 | N | PHE | 462 | 36.167 | 9.725 | −13.107 | 1.00 | 0.00 | 3A4 |
| ATOM | 3342 | CA | PHE | 462 | 34.983 | 8.975 | −13.533 | 1.00 | 0.00 | 3A4 |
| ATOM | 3343 | CB | PHE | 462 | 34.346 | 8.159 | −12.354 | 1.00 | 0.00 | 3A4 |
| ATOM | 3344 | CG | PHE | 462 | 35.274 | 7.073 | −11.873 | 1.00 | 0.00 | 3A4 |
| ATOM | 3345 | CD1 | PHE | 462 | 35.686 | 6.051 | −12.745 | 1.00 | 0.00 | 3A4 |
| ATOM | 3346 | CD2 | PHE | 462 | 35.718 | 7.025 | −10.540 | 1.00 | 0.00 | 3A4 |
| ATOM | 3347 | CE1 | PHE | 462 | 36.530 | 5.021 | −12.307 | 1.00 | 0.00 | 3A4 |
| ATOM | 3348 | CE2 | PHE | 462 | 36.561 | 6.001 | −10.089 | 1.00 | 0.00 | 3A4 |
| ATOM | 3349 | CZ | PHE | 462 | 36.972 | 5.002 | −10.978 | 1.00 | 0.00 | 3A4 |
| ATOM | 3350 | C | PHE | 462 | 33.836 | 9.848 | −14.135 | 1.00 | 0.00 | 3A4 |
| ATOM | 3351 | O | PHE | 462 | 33.734 | 11.037 | −13.828 | 1.00 | 0.00 | 3A4 |
| ATOM | 3352 | N | SER | 463 | 32.784 | 9.198 | −15.007 | 1.00 | 0.00 | 3A4 |
| ATOM | 3353 | CA | SER | 463 | 31.446 | 9.622 | −15.542 | 1.00 | 0.00 | 3A4 |
| ATOM | 3354 | CB | SER | 463 | 31.400 | 9.685 | −17.094 | 1.00 | 0.00 | 3A4 |
| ATOM | 3355 | OG | SER | 463 | 32.293 | 10.684 | −17.565 | 1.00 | 0.00 | 3A4 |
| ATOM | 3356 | C | SER | 463 | 30.513 | 8.581 | −15.014 | 1.00 | 0.00 | 3A4 |
| ATOM | 3357 | O | SER | 463 | 30.243 | 7.578 | −15.673 | 1.00 | 0.00 | 3A4 |
| ATOM | 3358 | N | PHE | 464 | 30.012 | 8.774 | −13.767 | 1.00 | 0.00 | 3A4 |
| ATOM | 3359 | CA | PHE | 464 | 29.251 | 7.754 | −13.078 | 1.00 | 0.00 | 3A4 |
| ATOM | 3360 | CB | PHE | 464 | 29.689 | 7.483 | −11.603 | 1.00 | 0.00 | 3A4 |
| ATOM | 3361 | CG | PHE | 464 | 29.600 | 8.582 | −10.565 | 1.00 | 0.00 | 3A4 |
| ATOM | 3362 | CD1 | PHE | 464 | 30.780 | 9.102 | −9.999 | 1.00 | 0.00 | 3A4 |
| ATOM | 3363 | CD2 | PHE | 464 | 28.364 | 8.952 | −9.990 | 1.00 | 0.00 | 3A4 |
| ATOM | 3364 | CE1 | PHE | 464 | 30.727 | 9.958 | −8.891 | 1.00 | 0.00 | 3A4 |
| ATOM | 3365 | CE2 | PHE | 464 | 28.310 | 9.832 | −8.899 | 1.00 | 0.00 | 3A4 |
| ATOM | 3366 | CZ | PHE | 464 | 29.493 | 10.333 | −8.347 | 1.00 | 0.00 | 3A4 |
| ATOM | 3367 | C | PHE | 464 | 27.768 | 7.948 | −13.199 | 1.00 | 0.00 | 3A4 |
| ATOM | 3368 | O | PHE | 464 | 27.238 | 9.056 | −13.175 | 1.00 | 0.00 | 3A4 |
| ATOM | 3369 | N | LYS | 465 | 27.076 | 6.801 | −13.331 | 1.00 | 0.00 | 3A4 |
| ATOM | 3370 | CA | LYS | 465 | 25.646 | 6.719 | −13.439 | 1.00 | 0.00 | 3A4 |
| ATOM | 3371 | CB | LYS | 465 | 25.197 | 6.398 | −14.891 | 1.00 | 0.00 | 3A4 |
| ATOM | 3372 | CG | LYS | 465 | 25.553 | 7.484 | −15.915 | 1.00 | 0.00 | 3A4 |
| ATOM | 3373 | CD | LYS | 465 | 25.067 | 7.162 | −17.333 | 1.00 | 0.00 | 3A4 |
| ATOM | 3374 | CE | LYS | 465 | 25.438 | 8.228 | −18.374 | 1.00 | 0.00 | 3A4 |
| ATOM | 3375 | NZ | LYS | 465 | 26.910 | 8.360 | −18.512 | 1.00 | 0.00 | 3A4 |
| ATOM | 3376 | C | LYS | 465 | 25.212 | 5.614 | −12.507 | 1.00 | 0.00 | 3A4 |
| ATOM | 3377 | O | LYS | 465 | 25.982 | 4.688 | −12.254 | 1.00 | 0.00 | 3A4 |
| ATOM | 3378 | N | PRO | 466 | 23.983 | 5.622 | −11.985 | 1.00 | 0.00 | 3A4 |
| ATOM | 3379 | CA | PRO | 466 | 23.422 | 4.511 | −11.249 | 1.00 | 0.00 | 3A4 |
| ATOM | 3380 | CD | PRO | 466 | 23.090 | 6.783 | −11.993 | 1.00 | 0.00 | 3A4 |
| ATOM | 3381 | CB | PRO | 466 | 22.300 | 5.151 | −10.405 | 1.00 | 0.00 | 3A4 |
| ATOM | 3382 | CG | PRO | 466 | 21.827 | 6.358 | −11.228 | 1.00 | 0.00 | 3A4 |
| ATOM | 3383 | C | PRO | 466 | 22.906 | 3.470 | −12.228 | 1.00 | 0.00 | 3A4 |
| ATOM | 3384 | O | PRO | 466 | 22.333 | 3.808 | −13.264 | 1.00 | 0.00 | 3A4 |
| ATOM | 3385 | N | CYS | 467 | 23.088 | 2.179 | −11.890 | 1.00 | 0.00 | 3A4 |
| ATOM | 3386 | CA | CYS | 467 | 22.624 | 1.049 | −12.667 | 1.00 | 0.00 | 3A4 |
| ATOM | 3387 | CB | CYS | 467 | 23.617 | −0.143 | −12.501 | 1.00 | 0.00 | 3A4 |
| ATOM | 3388 | SG | CYS | 467 | 23.434 | −1.534 | −13.674 | 1.00 | 0.00 | 3A4 |
| ATOM | 3389 | C | CYS | 467 | 21.212 | 0.659 | −12.220 | 1.00 | 0.00 | 3A4 |
| ATOM | 3390 | O | CYS | 467 | 20.555 | −0.179 | −12.834 | 1.00 | 0.00 | 3A4 |
| ATOM | 3391 | N | LYS | 468 | 20.726 | 1.325 | −11.130 | 1.00 | 0.00 | 3A4 |
| ATOM | 3392 | CA | LYS | 468 | 19.389 | 1.282 | −10.573 | 1.00 | 0.00 | 3A4 |
| ATOM | 3393 | CB | LYS | 468 | 19.406 | 1.410 | −9.027 | 1.00 | 0.00 | 3A4 |
| ATOM | 3394 | CG | LYS | 468 | 20.290 | 0.367 | −8.339 | 1.00 | 0.00 | 3A4 |
| ATOM | 3395 | CD | LYS | 468 | 20.136 | 0.413 | −6.821 | 1.00 | 0.00 | 3A4 |
| ATOM | 3396 | CE | LYS | 468 | 21.077 | −0.534 | −6.078 | 1.00 | 0.00 | 3A4 |
| ATOM | 3397 | NZ | LYS | 468 | 20.807 | −0.485 | −4.626 | 1.00 | 0.00 | 3A4 |
| ATOM | 3398 | C | LYS | 468 | 18.586 | 2.429 | −11.148 | 1.00 | 0.00 | 3A4 |
| ATOM | 3399 | O | LYS | 468 | 19.145 | 3.450 | −11.548 | 1.00 | 0.00 | 3A4 |
| ATOM | 3400 | N | GLU | 469 | 17.238 | 2.263 | −11.203 | 1.00 | 0.00 | 3A4 |
| ATOM | 3401 | CA | GLU | 469 | 16.301 | 3.177 | −11.832 | 1.00 | 0.00 | 3A4 |
| ATOM | 3402 | CB | GLU | 469 | 15.428 | 2.446 | −12.905 | 1.00 | 0.00 | 3A4 |
| ATOM | 3403 | CG | GLU | 469 | 16.234 | 1.719 | −14.000 | 1.00 | 0.00 | 3A4 |
| ATOM | 3404 | CD | GLU | 469 | 17.104 | 2.712 | −14.780 | 1.00 | 0.00 | 3A4 |
| ATOM | 3405 | OE1 | GLU | 469 | 16.530 | 3.642 | −15.407 | 1.00 | 0.00 | 3A4 |
| ATOM | 3406 | OE2 | GLU | 469 | 18.355 | 2.551 | −14.761 | 1.00 | 0.00 | 3A4 |
| ATOM | 3407 | C | GLU | 469 | 15.433 | 3.792 | −10.753 | 1.00 | 0.00 | 3A4 |
| ATOM | 3408 | O | GLU | 469 | 15.841 | 3.909 | −9.598 | 1.00 | 0.00 | 3A4 |
| ATOM | 3409 | N | THR | 470 | 14.183 | 4.182 | −11.133 | 1.00 | 0.00 | 3A4 |
| ATOM | 3410 | CA | THR | 470 | 13.125 | 4.722 | −10.289 | 1.00 | 0.00 | 3A4 |
| ATOM | 3411 | CB | THR | 470 | 12.400 | 5.877 | −10.998 | 1.00 | 0.00 | 3A4 |
| ATOM | 3412 | OG1 | THR | 470 | 11.519 | 6.597 | −10.138 | 1.00 | 0.00 | 3A4 |
| ATOM | 3413 | CG2 | THR | 470 | 11.657 | 5.427 | −12.286 | 1.00 | 0.00 | 3A4 |
| ATOM | 3414 | C | THR | 470 | 12.198 | 3.578 | −9.982 | 1.00 | 0.00 | 3A4 |
| ATOM | 3415 | O | THR | 470 | 11.456 | 3.677 | −8.906 | 1.00 | 0.00 | 3A4 |
| ATOM | 3416 | N | GLN | 471 | 12.279 | 2.447 | −10.640 | 1.00 | 0.00 | 3A4 |
| ATOM | 3417 | CA | GLN | 471 | 11.670 | 1.158 | −10.385 | 1.00 | 0.00 | 3A4 |
| ATOM | 3418 | CB | GLN | 471 | 10.997 | 0.559 | −11.656 | 1.00 | 0.00 | 3A4 |
| ATOM | 3419 | CG | GLN | 471 | 11.811 | 0.665 | −12.967 | 1.00 | 0.00 | 3A4 |
| ATOM | 3420 | CD | GLN | 471 | 10.976 | 0.103 | −14.119 | 1.00 | 0.00 | 3A4 |
| ATOM | 3421 | OE1 | GLN | 471 | 10.763 | −1.106 | −14.193 | 1.00 | 0.00 | 3A4 |
| ATOM | 3422 | NE2 | GLN | 471 | 10.489 | 0.985 | −15.035 | 1.00 | 0.00 | 3A4 |
| ATOM | 3423 | C | GLN | 471 | 12.773 | 0.276 | −9.839 | 1.00 | 0.00 | 3A4 |
| ATOM | 3424 | O | GLN | 471 | 13.605 | −0.249 | −10.577 | 1.00 | 0.00 | 3A4 |
| ATOM | 3425 | N | ILE | 472 | 12.809 | 0.178 | −8.475 | 1.00 | 0.00 | 3A4 |
| ATOM | 3426 | CA | ILE | 472 | 13.847 | −0.369 | −7.600 | 1.00 | 0.00 | 3A4 |
| ATOM | 3427 | CB | ILE | 472 | 14.451 | −1.737 | −7.993 | 1.00 | 0.00 | 3A4 |
| ATOM | 3428 | CG2 | ILE | 472 | 15.447 | −2.232 | −6.907 | 1.00 | 0.00 | 3A4 |
| ATOM | 3429 | CG1 | ILE | 472 | 13.359 | −2.820 | −8.240 | 1.00 | 0.00 | 3A4 |
| ATOM | 3430 | CD | ILE | 472 | 12.440 | −3.129 | −7.050 | 1.00 | 0.00 | 3A4 |
| ATOM | 3431 | C | ILE | 472 | 14.926 | 0.710 | −7.400 | 1.00 | 0.00 | 3A4 |
| ATOM | 3432 | O | ILE | 472 | 15.870 | 0.760 | −8.188 | 1.00 | 0.00 | 3A4 |
| ATOM | 3433 | N | PRO | 473 | 14.837 | 1.594 | −6.373 | 1.00 | 0.00 | 3A4 |
| ATOM | 3434 | CA | PRO | 473 | 15.865 | 2.553 | −5.978 | 1.00 | 0.00 | 3A4 |
| ATOM | 3435 | CD | PRO | 473 | 13.607 | 1.778 | −5.600 | 1.00 | 0.00 | 3A4 |
| ATOM | 3436 | CB | PRO | 473 | 15.047 | 3.657 | −5.263 | 1.00 | 0.00 | 3A4 |
| ATOM | 3437 | CG | PRO | 473 | 13.885 | 2.910 | −4.607 | 1.00 | 0.00 | 3A4 |
| ATOM | 3438 | C | PRO | 473 | 16.926 | 1.936 | −5.117 | 1.00 | 0.00 | 3A4 |
| ATOM | 3439 | O | PRO | 473 | 17.147 | 0.726 | −5.098 | 1.00 | 0.00 | 3A4 |
| ATOM | 3440 | N | LEU | 474 | 17.606 | 2.821 | −4.384 | 1.00 | 0.00 | 3A4 |
| ATOM | 3441 | CA | LEU | 474 | 18.692 | 2.518 | −3.506 | 1.00 | 0.00 | 3A4 |
| ATOM | 3442 | CB | LEU | 474 | 19.658 | 3.718 | −3.467 | 1.00 | 0.00 | 3A4 |
| ATOM | 3443 | CG | LEU | 474 | 20.935 | 3.548 | −2.623 | 1.00 | 0.00 | 3A4 |
| ATOM | 3444 | CD1 | LEU | 474 | 22.187 | 3.637 | −3.503 | 1.00 | 0.00 | 3A4 |
| ATOM | 3445 | CD2 | LEU | 474 | 20.962 | 4.569 | −1.472 | 1.00 | 0.00 | 3A4 |
| ATOM | 3446 | C | LEU | 474 | 18.157 | 2.198 | −2.136 | 1.00 | 0.00 | 3A4 |
| ATOM | 3447 | O | LEU | 474 | 17.397 | 2.966 | −1.547 | 1.00 | 0.00 | 3A4 |
| ATOM | 3448 | N | LYS | 475 | 18.561 | 1.016 | −1.612 | 1.00 | 0.00 | 3A4 |
| ATOM | 3449 | CA | LYS | 475 | 18.133 | 0.502 | −0.330 | 1.00 | 0.00 | 3A4 |
| ATOM | 3450 | CB | LYS | 475 | 18.199 | −1.049 | −0.249 | 1.00 | 0.00 | 3A4 |
| ATOM | 3451 | CG | LYS | 475 | 17.520 | −1.656 | 0.999 | 1.00 | 0.00 | 3A4 |
| ATOM | 3452 | CD | LYS | 475 | 17.579 | −3.189 | 1.074 | 1.00 | 0.00 | 3A4 |
| ATOM | 3453 | CE | LYS | 475 | 18.981 | −3.776 | 1.311 | 1.00 | 0.00 | 3A4 |
| ATOM | 3454 | NZ | LYS | 475 | 19.572 | −3.282 | 2.580 | 1.00 | 0.00 | 3A4 |
| ATOM | 3455 | C | LYS | 475 | 18.961 | 1.086 | 0.775 | 1.00 | 0.00 | 3A4 |
| ATOM | 3456 | O | LYS | 475 | 20.173 | 0.909 | 0.825 | 1.00 | 0.00 | 3A4 |
| ATOM | 3457 | N | LEU | 476 | 18.276 | 1.798 | 1.695 | 1.00 | 0.00 | 3A4 |
| ATOM | 3458 | CA | LEU | 476 | 18.860 | 2.419 | 2.855 | 1.00 | 0.00 | 3A4 |
| ATOM | 3459 | CB | LEU | 476 | 18.422 | 3.903 | 3.070 | 1.00 | 0.00 | 3A4 |
| ATOM | 3460 | CG | LEU | 476 | 16.930 | 4.243 | 3.360 | 1.00 | 0.00 | 3A4 |
| ATOM | 3461 | CD1 | LEU | 476 | 16.813 | 5.725 | 3.764 | 1.00 | 0.00 | 3A4 |
| ATOM | 3462 | CD2 | LEU | 476 | 15.956 | 3.930 | 2.201 | 1.00 | 0.00 | 3A4 |
| ATOM | 3463 | C | LEU | 476 | 18.580 | 1.579 | 4.038 | 1.00 | 0.00 | 3A4 |
| ATOM | 3464 | O | LEU | 476 | 17.524 | 0.970 | 4.149 | 1.00 | 0.00 | 3A4 |
| ATOM | 3465 | N | SER | 477 | 19.543 | 1.495 | 4.973 | 1.00 | 0.00 | 3A4 |
| ATOM | 3466 | CA | SER | 477 | 19.480 | 0.545 | 6.040 | 1.00 | 0.00 | 3A4 |
| ATOM | 3467 | CB | SER | 477 | 20.807 | −0.220 | 6.343 | 1.00 | 0.00 | 3A4 |
| ATOM | 3468 | OG | SER | 477 | 20.584 | −1.554 | 6.795 | 1.00 | 0.00 | 3A4 |
| ATOM | 3469 | C | SER | 477 | 18.826 | 1.150 | 7.228 | 1.00 | 0.00 | 3A4 |
| ATOM | 3470 | O | SER | 477 | 18.936 | 2.334 | 7.525 | 1.00 | 0.00 | 3A4 |
| ATOM | 3471 | N | LEU | 478 | 18.068 | 0.282 | 7.917 | 1.00 | 0.00 | 3A4 |
| ATOM | 3472 | CA | LEU | 478 | 17.386 | 0.588 | 9.132 | 1.00 | 0.00 | 3A4 |
| ATOM | 3473 | CB | LEU | 478 | 16.072 | −0.245 | 9.288 | 1.00 | 0.00 | 3A4 |
| ATOM | 3474 | CG | LEU | 478 | 15.314 | −0.208 | 10.653 | 1.00 | 0.00 | 3A4 |
| ATOM | 3475 | CD1 | LEU | 478 | 14.954 | 1.206 | 11.152 | 1.00 | 0.00 | 3A4 |
| ATOM | 3476 | CD2 | LEU | 478 | 14.057 | −1.098 | 10.607 | 1.00 | 0.00 | 3A4 |
| ATOM | 3477 | C | LEU | 478 | 18.338 | 0.280 | 10.281 | 1.00 | 0.00 | 3A4 |
| ATOM | 3478 | O | LEU | 478 | 18.566 | −0.855 | 10.665 | 1.00 | 0.00 | 3A4 |
| ATOM | 3479 | N | GLY | 479 | 18.982 | 1.308 | 10.841 | 1.00 | 0.00 | 3A4 |
| ATOM | 3480 | CA | GLY | 479 | 19.983 | 1.119 | 11.875 | 1.00 | 0.00 | 3A4 |
| ATOM | 3481 | C | GLY | 479 | 19.558 | 1.665 | 13.192 | 1.00 | 0.00 | 3A4 |
| ATOM | 3482 | O | GLY | 479 | 19.405 | 0.936 | 14.154 | 1.00 | 0.00 | 3A4 |
| ATOM | 3483 | N | GLY | 480 | 19.416 | 2.996 | 13.329 | 1.00 | 0.00 | 3A4 |
| ATOM | 3484 | CA | GLY | 480 | 19.205 | 3.607 | 14.623 | 1.00 | 0.00 | 3A4 |
| ATOM | 3485 | C | GLY | 480 | 20.442 | 4.355 | 14.943 | 1.00 | 0.00 | 3A4 |
| ATOM | 3486 | O | GLY | 480 | 20.430 | 5.574 | 14.961 | 1.00 | 0.00 | 3A4 |
| ATOM | 3487 | N | LEU | 481 | 21.566 | 3.643 | 15.196 | 1.00 | 0.00 | 3A4 |
| ATOM | 3488 | CA | LEU | 481 | 22.833 | 4.218 | 15.597 | 1.00 | 0.00 | 3A4 |
| ATOM | 3489 | CB | LEU | 481 | 23.634 | 3.303 | 16.586 | 1.00 | 0.00 | 3A4 |
| ATOM | 3490 | CG | LEU | 481 | 23.971 | 1.830 | 16.194 | 1.00 | 0.00 | 3A4 |
| ATOM | 3491 | CD1 | LEU | 481 | 25.057 | 1.273 | 17.135 | 1.00 | 0.00 | 3A4 |
| ATOM | 3492 | CD2 | LEU | 481 | 22.767 | 0.861 | 16.168 | 1.00 | 0.00 | 3A4 |
| ATOM | 3493 | C | LEU | 481 | 23.664 | 4.637 | 14.398 | 1.00 | 0.00 | 3A4 |
| ATOM | 3494 | O | LEU | 481 | 24.121 | 5.774 | 14.349 | 1.00 | 0.00 | 3A4 |
| ATOM | 3495 | N | LEU | 482 | 23.838 | 3.733 | 13.398 | 1.00 | 0.00 | 3A4 |
| ATOM | 3496 | CA | LEU | 482 | 24.486 | 3.984 | 12.124 | 1.00 | 0.00 | 3A4 |
| ATOM | 3497 | CB | LEU | 482 | 25.613 | 2.951 | 11.765 | 1.00 | 0.00 | 3A4 |
| ATOM | 3498 | CG | LEU | 482 | 26.965 | 3.067 | 12.517 | 1.00 | 0.00 | 3A4 |
| ATOM | 3499 | CD1 | LEU | 482 | 27.722 | 4.365 | 12.213 | 1.00 | 0.00 | 3A4 |
| ATOM | 3500 | CD2 | LEU | 482 | 26.897 | 2.808 | 14.028 | 1.00 | 0.00 | 3A4 |
| ATOM | 3501 | C | LEU | 482 | 23.378 | 3.835 | 11.115 | 1.00 | 0.00 | 3A4 |
| ATOM | 3502 | O | LEU | 482 | 22.784 | 2.766 | 11.044 | 1.00 | 0.00 | 3A4 |
| ATOM | 3503 | N | GLN | 483 | 23.090 | 4.887 | 10.291 | 1.00 | 0.00 | 3A4 |
| ATOM | 3504 | CA | GLN | 483 | 22.131 | 4.849 | 9.194 | 1.00 | 0.00 | 3A4 |
| ATOM | 3505 | CB | GLN | 483 | 21.199 | 6.105 | 9.194 | 1.00 | 0.00 | 3A4 |
| ATOM | 3506 | CG | GLN | 483 | 20.316 | 6.274 | 10.449 | 1.00 | 0.00 | 3A4 |
| ATOM | 3507 | CD | GLN | 483 | 19.134 | 5.295 | 10.458 | 1.00 | 0.00 | 3A4 |
| ATOM | 3508 | OE1 | GLN | 483 | 18.982 | 4.423 | 9.605 | 1.00 | 0.00 | 3A4 |
| ATOM | 3509 | NE2 | GLN | 483 | 18.236 | 5.459 | 11.467 | 1.00 | 0.00 | 3A4 |
| ATOM | 3510 | C | GLN | 483 | 22.916 | 4.815 | 7.900 | 1.00 | 0.00 | 3A4 |
| ATOM | 3511 | O | GLN | 483 | 23.541 | 5.820 | 7.606 | 1.00 | 0.00 | 3A4 |
| ATOM | 3512 | N | PRO | 484 | 22.951 | 3.748 | 7.093 | 1.00 | 0.00 | 3A4 |
| ATOM | 3513 | CA | PRO | 484 | 23.817 | 3.721 | 5.925 | 1.00 | 0.00 | 3A4 |
| ATOM | 3514 | CD | PRO | 484 | 22.874 | 2.409 | 7.687 | 1.00 | 0.00 | 3A4 |
| ATOM | 3515 | CB | PRO | 484 | 24.769 | 2.558 | 6.262 | 1.00 | 0.00 | 3A4 |
| ATOM | 3516 | CG | PRO | 484 | 23.861 | 1.520 | 6.915 | 1.00 | 0.00 | 3A4 |
| ATOM | 3517 | C | PRO | 484 | 23.035 | 3.412 | 4.673 | 1.00 | 0.00 | 3A4 |
| ATOM | 3518 | O | PRO | 484 | 21.816 | 3.307 | 4.680 | 1.00 | 0.00 | 3A4 |
| ATOM | 3519 | N | GLU | 485 | 23.773 | 3.212 | 3.562 | 1.00 | 0.00 | 3A4 |
| ATOM | 3520 | CA | GLU | 485 | 23.248 | 2.558 | 2.383 | 1.00 | 0.00 | 3A4 |
| ATOM | 3521 | CB | GLU | 485 | 23.098 | 3.493 | 1.177 | 1.00 | 0.00 | 3A4 |
| ATOM | 3522 | CG | GLU | 485 | 24.327 | 4.316 | 0.781 | 1.00 | 0.00 | 3A4 |
| ATOM | 3523 | CD | GLU | 485 | 24.829 | 3.771 | −0.540 | 1.00 | 0.00 | 3A4 |
| ATOM | 3524 | OE1 | GLU | 485 | 24.837 | 4.541 | −1.536 | 1.00 | 0.00 | 3A4 |
| ATOM | 3525 | OE2 | GLU | 485 | 25.209 | 2.571 | −0.567 | 1.00 | 0.00 | 3A4 |
| ATOM | 3526 | C | GLU | 485 | 24.043 | 1.288 | 2.206 | 1.00 | 0.00 | 3A4 |
| ATOM | 3527 | O | GLU | 485 | 25.260 | 1.289 | 2.296 | 1.00 | 0.00 | 3A4 |
| ATOM | 3528 | N | LYS | 486 | 23.368 | 0.126 | 2.027 | 1.00 | 0.00 | 3A4 |
| ATOM | 3529 | CA | LYS | 486 | 24.038 | −1.172 | 1.973 | 1.00 | 0.00 | 3A4 |
| ATOM | 3530 | CB | LYS | 486 | 23.286 | −2.272 | 2.791 | 1.00 | 0.00 | 3A4 |
| ATOM | 3531 | CG | LYS | 486 | 24.070 | −3.558 | 3.141 | 1.00 | 0.00 | 3A4 |
| ATOM | 3532 | CD | LYS | 486 | 24.136 | −4.672 | 2.074 | 1.00 | 0.00 | 3A4 |
| ATOM | 3533 | CE | LYS | 486 | 22.782 | −5.249 | 1.626 | 1.00 | 0.00 | 3A4 |
| ATOM | 3534 | NZ | LYS | 486 | 22.037 | −5.839 | 2.765 | 1.00 | 0.00 | 3A4 |
| ATOM | 3535 | C | LYS | 486 | 24.427 | −1.507 | 0.545 | 1.00 | 0.00 | 3A4 |
| ATOM | 3536 | O | LYS | 486 | 25.595 | −1.841 | 0.334 | 1.00 | 0.00 | 3A4 |
| ATOM | 3537 | N | PRO | 487 | 23.564 | −1.396 | −0.475 | 1.00 | 0.00 | 3A4 |
| ATOM | 3538 | CA | PRO | 487 | 23.980 | −1.646 | −1.846 | 1.00 | 0.00 | 3A4 |
| ATOM | 3539 | CD | PRO | 487 | 22.127 | −1.666 | −0.334 | 1.00 | 0.00 | 3A4 |
| ATOM | 3540 | CB | PRO | 487 | 22.967 | −2.687 | −2.354 | 1.00 | 0.00 | 3A4 |
| ATOM | 3541 | CG | PRO | 487 | 21.658 | −2.329 | −1.638 | 1.00 | 0.00 | 3A4 |
| ATOM | 3542 | C | PRO | 487 | 23.927 | −0.353 | −2.647 | 1.00 | 0.00 | 3A4 |
| ATOM | 3543 | O | PRO | 487 | 22.999 | 0.443 | −2.510 | 1.00 | 0.00 | 3A4 |
| ATOM | 3544 | N | VAL | 488 | 24.915 | −0.147 | −3.541 | 1.00 | 0.00 | 3A4 |
| ATOM | 3545 | CA | VAL | 488 | 24.946 | 1.001 | −4.417 | 1.00 | 0.00 | 3A4 |
| ATOM | 3546 | CB | VAL | 488 | 25.614 | 2.234 | −3.794 | 1.00 | 0.00 | 3A4 |
| ATOM | 3547 | CG1 | VAL | 488 | 26.927 | 1.920 | −3.030 | 1.00 | 0.00 | 3A4 |
| ATOM | 3548 | CG2 | VAL | 488 | 25.731 | 3.431 | −4.776 | 1.00 | 0.00 | 3A4 |
| ATOM | 3549 | C | VAL | 488 | 25.663 | 0.556 | −5.657 | 1.00 | 0.00 | 3A4 |
| ATOM | 3550 | O | VAL | 488 | 26.886 | 0.485 | −5.692 | 1.00 | 0.00 | 3A4 |
| ATOM | 3551 | N | VAL | 489 | 24.905 | 0.264 | −6.743 | 1.00 | 0.00 | 3A4 |
| ATOM | 3552 | CA | VAL | 489 | 25.452 | −0.202 | −8.007 | 1.00 | 0.00 | 3A4 |
| ATOM | 3553 | CB | VAL | 489 | 24.587 | −1.266 | −8.687 | 1.00 | 0.00 | 3A4 |
| ATOM | 3554 | CG1 | VAL | 489 | 25.369 | −1.938 | −9.848 | 1.00 | 0.00 | 3A4 |
| ATOM | 3555 | CG2 | VAL | 489 | 24.212 | −2.339 | −7.638 | 1.00 | 0.00 | 3A4 |
| ATOM | 3556 | C | VAL | 489 | 25.702 | 0.999 | −8.916 | 1.00 | 0.00 | 3A4 |
| ATOM | 3557 | O | VAL | 489 | 24.854 | 1.864 | −9.093 | 1.00 | 0.00 | 3A4 |
| ATOM | 3558 | N | LEU | 490 | 26.915 | 1.064 | −9.499 | 1.00 | 0.00 | 3A4 |
| ATOM | 3559 | CA | LEU | 490 | 27.387 | 2.104 | −10.386 | 1.00 | 0.00 | 3A4 |
| ATOM | 3560 | CB | LEU | 490 | 28.695 | 2.765 | −9.828 | 1.00 | 0.00 | 3A4 |
| ATOM | 3561 | CG | LEU | 490 | 29.556 | 3.732 | −10.717 | 1.00 | 0.00 | 3A4 |
| ATOM | 3562 | CD1 | LEU | 490 | 30.364 | 4.675 | −9.816 | 1.00 | 0.00 | 3A4 |
| ATOM | 3563 | CD2 | LEU | 490 | 30.579 | 3.092 | −11.694 | 1.00 | 0.00 | 3A4 |
| ATOM | 3564 | C | LEU | 490 | 27.689 | 1.443 | −11.710 | 1.00 | 0.00 | 3A4 |
| ATOM | 3565 | O | LEU | 490 | 28.311 | 0.382 | −11.714 | 1.00 | 0.00 | 3A4 |
| ATOM | 3566 | N | LYS | 491 | 27.223 | 2.080 | −12.824 | 1.00 | 0.00 | 3A4 |
| ATOM | 3567 | CA | LYS | 491 | 27.177 | 1.589 | −14.188 | 1.00 | 0.00 | 3A4 |
| ATOM | 3568 | CB | LYS | 491 | 25.982 | 2.230 | −14.965 | 1.00 | 0.00 | 3A4 |
| ATOM | 3569 | CG | LYS | 491 | 25.687 | 1.704 | −16.388 | 1.00 | 0.00 | 3A4 |
| ATOM | 3570 | CD | LYS | 491 | 25.319 | 0.219 | −16.460 | 1.00 | 0.00 | 3A4 |
| ATOM | 3571 | CE | LYS | 491 | 25.043 | −0.263 | −17.889 | 1.00 | 0.00 | 3A4 |
| ATOM | 3572 | NZ | LYS | 491 | 24.678 | −1.699 | −17.899 | 1.00 | 0.00 | 3A4 |
| ATOM | 3573 | C | LYS | 491 | 28.494 | 1.812 | −14.912 | 1.00 | 0.00 | 3A4 |
| ATOM | 3574 | O | LYS | 491 | 29.380 | 0.964 | −14.854 | 1.00 | 0.00 | 3A4 |
| ATOM | 3575 | N | VAL | 492 | 28.641 | 2.966 | −15.620 | 1.00 | 0.00 | 3A4 |
| ATOM | 3576 | CA | VAL | 492 | 29.700 | 3.255 | −16.574 | 1.00 | 0.00 | 3A4 |
| ATOM | 3577 | CB | VAL | 492 | 29.112 | 3.889 | −17.838 | 1.00 | 0.00 | 3A4 |
| ATOM | 3578 | CG1 | VAL | 492 | 28.415 | 5.242 | −17.560 | 1.00 | 0.00 | 3A4 |
| ATOM | 3579 | CG2 | VAL | 492 | 30.130 | 3.946 | −19.001 | 1.00 | 0.00 | 3A4 |
| ATOM | 3580 | C | VAL | 492 | 30.777 | 4.082 | −15.898 | 1.00 | 0.00 | 3A4 |
| ATOM | 3581 | O | VAL | 492 | 30.519 | 4.769 | −14.911 | 1.00 | 0.00 | 3A4 |
| ATOM | 3582 | N | GLU | 493 | 32.030 | 3.983 | −16.415 | 1.00 | 0.00 | 3A4 |
| ATOM | 3583 | CA | GLU | 493 | 33.215 | 4.574 | −15.836 | 1.00 | 0.00 | 3A4 |
| ATOM | 3584 | CB | GLU | 493 | 33.962 | 3.566 | −14.901 | 1.00 | 0.00 | 3A4 |
| ATOM | 3585 | CG | GLU | 493 | 34.397 | 2.206 | −15.502 | 1.00 | 0.00 | 3A4 |
| ATOM | 3586 | CD | GLU | 493 | 35.792 | 2.272 | −16.141 | 1.00 | 0.00 | 3A4 |
| ATOM | 3587 | OE1 | GLU | 493 | 36.769 | 2.568 | −15.402 | 1.00 | 0.00 | 3A4 |
| ATOM | 3588 | OE2 | GLU | 493 | 35.901 | 2.024 | −17.372 | 1.00 | 0.00 | 3A4 |
| ATOM | 3589 | C | GLU | 493 | 34.100 | 5.031 | −16.967 | 1.00 | 0.00 | 3A4 |
| ATOM | 3590 | O | GLU | 493 | 33.928 | 4.629 | −18.117 | 1.00 | 0.00 | 3A4 |
| ATOM | 3591 | N | SER | 494 | 35.109 | 5.874 | −16.624 | 1.00 | 0.00 | 3A4 |
| ATOM | 3592 | CA | SER | 494 | 36.209 | 6.283 | −17.480 | 1.00 | 0.00 | 3A4 |
| ATOM | 3593 | CB | SER | 494 | 36.076 | 7.748 | −18.010 | 1.00 | 0.00 | 3A4 |
| ATOM | 3594 | OG | SER | 494 | 35.765 | 8.692 | −16.990 | 1.00 | 0.00 | 3A4 |
| ATOM | 3595 | C | SER | 494 | 37.455 | 6.059 | −16.647 | 1.00 | 0.00 | 3A4 |
| ATOM | 3596 | O | SER | 494 | 37.360 | 5.784 | −15.452 | 1.00 | 0.00 | 3A4 |
| ATOM | 3597 | N | ARG | 495 | 38.664 | 6.138 | −17.262 | 1.00 | 0.00 | 3A4 |
| ATOM | 3598 | CA | ARG | 495 | 39.886 | 5.740 | −16.587 | 1.00 | 0.00 | 3A4 |
| ATOM | 3599 | CB | ARG | 495 | 40.090 | 4.192 | −16.590 | 1.00 | 0.00 | 3A4 |
| ATOM | 3600 | CG | ARG | 495 | 39.845 | 3.452 | −17.916 | 1.00 | 0.00 | 3A4 |
| ATOM | 3601 | CD | ARG | 495 | 39.764 | 1.935 | −17.697 | 1.00 | 0.00 | 3A4 |
| ATOM | 3602 | NE | ARG | 495 | 39.301 | 1.272 | −18.966 | 1.00 | 0.00 | 3A4 |
| ATOM | 3603 | CZ | ARG | 495 | 38.710 | 0.032 | −18.992 | 1.00 | 0.00 | 3A4 |
| ATOM | 3604 | NH1 | ARG | 495 | 38.289 | −0.478 | −20.185 | 1.00 | 0.00 | 3A4 |
| ATOM | 3605 | NH2 | ARG | 495 | 38.527 | −0.702 | −17.853 | 1.00 | 0.00 | 3A4 |
| ATOM | 3606 | C | ARG | 495 | 41.073 | 6.436 | −17.202 | 1.00 | 0.00 | 3A4 |
| ATOM | 3607 | O | ARG | 495 | 41.144 | 6.643 | −18.413 | 1.00 | 0.00 | 3A4 |
| ATOM | 3608 | N | ASP | 496 | 42.044 | 6.804 | −16.320 | 1.00 | 0.00 | 3A4 |
| ATOM | 3609 | CA | ASP | 496 | 43.290 | 7.471 | −16.647 | 1.00 | 0.00 | 3A4 |
| ATOM | 3610 | CB | ASP | 496 | 43.159 | 9.025 | −16.860 | 1.00 | 0.00 | 3A4 |
| ATOM | 3611 | CG | ASP | 496 | 42.410 | 9.788 | −15.743 | 1.00 | 0.00 | 3A4 |
| ATOM | 3612 | OD1 | ASP | 496 | 43.070 | 10.614 | −15.056 | 1.00 | 0.00 | 3A4 |
| ATOM | 3613 | OD2 | ASP | 496 | 41.179 | 9.574 | −15.577 | 1.00 | 0.00 | 3A4 |
| ATOM | 3614 | C | ASP | 496 | 44.272 | 7.071 | −15.553 | 1.00 | 0.00 | 3A4 |
| ATOM | 3615 | O | ASP | 496 | 44.432 | 5.882 | −15.283 | 1.00 | 0.00 | 3A4 |
| ATOM | 3616 | N | GLY | 497 | 44.958 | 8.050 | −14.898 | 1.00 | 0.00 | 3A4 |
| ATOM | 3617 | CA | GLY | 497 | 45.950 | 7.790 | −13.872 | 1.00 | 0.00 | 3A4 |
| ATOM | 3618 | C | GLY | 497 | 46.123 | 9.024 | −13.031 | 1.00 | 0.00 | 3A4 |
| ATOM | 3619 | O | GLY | 497 | 46.262 | 10.127 | −13.557 | 1.00 | 0.00 | 3A4 |
| ATOM | 3620 | N | THR | 498 | 46.127 | 8.846 | −11.677 | 1.00 | 0.00 | 3A4 |
| ATOM | 3621 | CA | THR | 498 | 46.355 | 9.879 | −10.674 | 1.00 | 0.00 | 3A4 |
| ATOM | 3622 | CB | THR | 498 | 45.075 | 10.401 | −9.995 | 1.00 | 0.00 | 3A4 |
| ATOM | 3623 | OG1 | THR | 498 | 44.194 | 9.349 | −9.600 | 1.00 | 0.00 | 3A4 |
| ATOM | 3624 | CG2 | THR | 498 | 44.332 | 11.355 | −10.957 | 1.00 | 0.00 | 3A4 |
| ATOM | 3625 | C | THR | 498 | 47.290 | 9.274 | −9.640 | 1.00 | 0.00 | 3A4 |
| ATOM | 3626 | O | THR | 498 | 47.189 | 8.089 | −9.326 | 1.00 | 0.00 | 3A4 |
| ATOM | 3627 | N | VAL | 499 | 48.205 | 10.109 | −9.074 | 1.00 | 0.00 | 3A4 |
| ATOM | 3628 | CA | VAL | 499 | 49.121 | 9.727 | −8.014 | 1.00 | 0.00 | 3A4 |
| ATOM | 3629 | CB | VAL | 499 | 50.272 | 8.816 | −8.476 | 1.00 | 0.00 | 3A4 |
| ATOM | 3630 | CG1 | VAL | 499 | 51.133 | 9.436 | −9.607 | 1.00 | 0.00 | 3A4 |
| ATOM | 3631 | CG2 | VAL | 499 | 51.110 | 8.313 | −7.273 | 1.00 | 0.00 | 3A4 |
| ATOM | 3632 | C | VAL | 499 | 49.614 | 11.021 | −7.391 | 1.00 | 0.00 | 3A4 |
| ATOM | 3633 | O | VAL | 499 | 49.977 | 11.961 | −8.097 | 1.00 | 0.00 | 3A4 |
| ATOM | 3634 | N | SER | 500 | 49.647 | 11.075 | −6.030 | 1.00 | 0.00 | 3A4 |
| ATOM | 3635 | CA | SER | 500 | 50.251 | 12.152 | −5.269 | 1.00 | 0.00 | 3A4 |
| ATOM | 3636 | CB | SER | 500 | 49.434 | 13.486 | −5.230 | 1.00 | 0.00 | 3A4 |
| ATOM | 3637 | OG | SER | 500 | 48.086 | 13.306 | −4.816 | 1.00 | 0.00 | 3A4 |
| ATOM | 3638 | C | SER | 500 | 50.524 | 11.618 | −3.882 | 1.00 | 0.00 | 3A4 |
| ATOM | 3639 | O | SER | 500 | 49.694 | 10.933 | −3.286 | 1.00 | 0.00 | 3A4 |
| ATOM | 3640 | N | GLY | 501 | 51.736 | 11.945 | −3.355 | 1.00 | 0.00 | 3A4 |
| ATOM | 3641 | CA | GLY | 501 | 52.249 | 11.543 | −2.061 | 1.00 | 0.00 | 3A4 |
| ATOM | 3642 | C | GLY | 501 | 53.489 | 10.727 | −2.291 | 1.00 | 0.00 | 3A4 |
| ATOM | 3643 | O | GLY | 501 | 54.576 | 11.087 | −1.841 | 1.00 | 0.00 | 3A4 |
| ATOM | 3644 | N | ALA | 502 | 53.331 | 9.596 | −3.020 | 1.00 | 0.00 | 3A4 |
| ATOM | 3645 | CA | ALA | 502 | 54.406 | 8.722 | −3.424 | 1.00 | 0.00 | 3A4 |
| ATOM | 3646 | CB | ALA | 502 | 54.772 | 7.642 | −2.379 | 1.00 | 0.00 | 3A4 |
| ATOM | 3647 | C | ALA | 502 | 53.912 | 8.031 | −4.704 | 1.00 | 0.00 | 3A4 |
| ATOM | 3648 | OT1 | ALA | 502 | 54.439 | 8.364 | −5.800 | 1.00 | 0.00 | 3A4 |
| ATOM | 3649 | OT2 | ALA | 502 | 52.987 | 7.178 | −4.607 | 1.00 | 0.00 | 3A4 |
| TER | 3650 | | ALA | 502 | | | | | | |
| HETATM | 3651 | FE | HEM | 600 | 33.118 | 10.391 | 15.288 | 1.00 | 0.00 | HEM |
| HETATM | 3652 | NA | HEM | 600 | 31.497 | 11.115 | 16.171 | 1.00 | 0.00 | HEM |
| HETATM | 3653 | NB | HEM | 600 | 32.274 | 10.658 | 13.514 | 1.00 | 0.00 | HEM |
| HETATM | 3654 | NC | HEM | 600 | 34.752 | 9.684 | 14.430 | 1.00 | 0.00 | HEM |
| HETATM | 3655 | ND | HEM | 600 | 33.949 | 10.106 | 17.065 | 1.00 | 0.00 | HEM |
| HETATM | 3656 | C1A | HEM | 600 | 31.254 | 11.251 | 17.515 | 1.00 | 0.00 | HEM |
| HETATM | 3657 | C2A | HEM | 600 | 29.887 | 11.659 | 17.752 | 1.00 | 0.00 | HEM |
| HETATM | 3658 | C3A | HEM | 600 | 29.316 | 11.871 | 16.542 | 1.00 | 0.00 | HEM |
| HETATM | 3659 | C4A | HEM | 600 | 30.322 | 11.523 | 15.568 | 1.00 | 0.00 | HEM |
| HETATM | 3660 | C1B | HEM | 600 | 31.012 | 11.136 | 13.233 | 1.00 | 0.00 | HEM |
| HETATM | 3661 | C2B | HEM | 600 | 30.761 | 11.218 | 11.804 | 1.00 | 0.00 | HEM |
| HETATM | 3662 | C3B | HEM | 600 | 31.901 | 10.761 | 11.185 | 1.00 | 0.00 | HEM |
| HETATM | 3663 | C4B | HEM | 600 | 32.828 | 10.426 | 12.273 | 1.00 | 0.00 | HEM |
| HETATM | 3664 | C1C | HEM | 600 | 35.044 | 9.645 | 13.089 | 1.00 | 0.00 | HEM |
| HETATM | 3665 | C2C | HEM | 600 | 36.395 | 9.168 | 12.838 | 1.00 | 0.00 | HEM |
| HETATM | 3666 | C3C | HEM | 600 | 36.920 | 8.833 | 14.067 | 1.00 | 0.00 | HEM |
| HETATM | 3667 | C4C | HEM | 600 | 35.879 | 9.167 | 15.033 | 1.00 | 0.00 | HEM |
| HETATM | 3668 | C1D | HEM | 600 | 35.150 | 9.494 | 17.358 | 1.00 | 0.00 | HEM |
| HETATM | 3669 | C2D | HEM | 600 | 35.382 | 9.408 | 18.787 | 1.00 | 0.00 | HEM |
| HETATM | 3670 | C3D | HEM | 600 | 34.329 | 10.035 | 19.375 | 1.00 | 0.00 | HEM |
| HETATM | 3671 | C4D | HEM | 600 | 33.438 | 10.439 | 18.306 | 1.00 | 0.00 | HEM |
| HETATM | 3672 | CHA | HEM | 600 | 32.186 | 10.978 | 18.500 | 1.00 | 0.00 | HEM |
| HETATM | 3673 | CHB | HEM | 600 | 30.115 | 11.530 | 14.204 | 1.00 | 0.00 | HEM |
| HETATM | 3674 | CHC | HEM | 600 | 34.131 | 9.963 | 12.102 | 1.00 | 0.00 | HEM |
| HETATM | 3675 | CHD | HEM | 600 | 36.032 | 9.062 | 16.407 | 1.00 | 0.00 | HEM |
| HETATM | 3676 | CMA | HEM | 600 | 27.911 | 12.344 | 16.281 | 1.00 | 0.00 | HEM |
| HETATM | 3677 | CAA | HEM | 600 | 29.208 | 11.695 | 19.088 | 1.00 | 0.00 | HEM |
| HETATM | 3678 | CBA | HEM | 600 | 29.154 | 13.045 | 19.638 | 1.00 | 0.90 | HEM |
| HETATM | 3679 | CGA | HEM | 600 | 28.459 | 13.237 | 20.946 | 1.00 | 0.00 | HEM |
| HETATM | 3680 | O1A | HEM | 600 | 28.097 | 12.278 | 21.566 | 1.00 | 0.00 | HEM |
| HETATM | 3681 | O2A | HEM | 600 | 28.217 | 14.323 | 21.438 | 1.00 | 0.00 | HEM |
| HETATM | 3682 | CMB | HEM | 600 | 29.483 | 11.742 | 11.185 | 1.00 | 0.00 | HEM |
| HETATM | 3683 | CAB | HEM | 600 | 32.219 | 10.602 | 9.818 | 1.00 | 0.00 | HEM |
| HETATM | 3684 | CBB | HEM | 600 | 31.527 | 10.978 | 8.735 | 1.00 | 0.00 | HEM |
| HETATM | 3685 | CMC | HEM | 600 | 37.047 | 9.116 | 11.471 | 1.00 | 0.00 | HEM |
| HETATM | 3686 | CAC | HEM | 600 | 38.159 | 8.288 | 14.465 | 1.00 | 0.00 | HEM |
| HETATM | 3687 | CBC | HEM | 600 | 39.265 | 8.069 | 13.758 | 1.00 | 0.00 | HEM |
| HETATM | 3688 | CMD | HEM | 600 | 36.499 | 8.680 | 19.483 | 1.00 | 0.00 | HEM |
| HETATM | 3689 | CAD | HEM | 600 | 34.101 | 10.253 | 20.849 | 1.00 | 0.00 | HEM |
| HETATM | 3690 | CBD | HEM | 600 | 34.689 | 11.583 | 21.366 | 1.00 | 0.00 | HEM |
| HETATM | 3691 | CGD | HEM | 600 | 34.355 | 11.863 | 22.843 | 1.00 | 0.00 | HEM |
| HETATM | 3692 | O1D | HEM | 600 | 35.050 | 12.735 | 23.424 | 1.00 | 0.00 | HEM |
| HETATM | 3693 | O2D | HEM | 600 | 33.381 | 11.284 | 23.380 | 1.00 | 0.00 | HEM |
| END | | | | | | | | | | |

TABLE 4
Providing the coordinates of the CYP3A model

| HEADER | CYP3A7 | | | | | | | | | | | | | | |
|---|---|---|---|---|---|---|---|---|---|---|---|---|---|---|---|
| TITLE | MODEL OF HUMAN CYTOCHROME P450 CYP3A7 | | | | | | | | | | | | | | |
| AUTHOR | N. LOISEAU, F. ANDRE, M. DELAFORGE, M. COTTEVIEILLE | | | | | | | | | | | | | | |
| SEQRES | 1 | 459 | PRO | PHE | LEU | GLY | ASN | ALA | LEU | SER | PHE | ARG | LYS | GLY | TYR |
| SEQRES | 2 | 459 | TRP | THR | PHE | ASP | MET | GLU | CYS | TYR | LYS | LYS | TYR | ARG | LYS |
| SEQRES | 3 | 459 | VAL | TRP | GLY | ILE | TYR | ASP | CYS | GLN | GLN | PRO | MET | LEU | ALA |
| SEQRES | 4 | 459 | ILE | THR | ASP | PRO | ASP | MET | ILE | LYS | THR | VAL | LEU | VAL | LYS |
| SEQRES | 5 | 459 | GLU | CYS | TYR | SER | VAL | PHE | THR | ASN | ARG | ARG | PRO | PHE | GLY |
| SEQRES | 6 | 459 | PRO | VAL | GLY | PHE | MET | LYS | ASN | ALA | ILE | SER | ILE | ALA | GLU |
| SEQRES | 7 | 459 | ASP | GLU | GLU | TRP | LYS | ARG | ILE | ARG | SER | LEU | LEU | SER | PRO |
| SEQRES | 8 | 459 | THR | PHE | THR | SER | GLY | LYS | LEU | LYS | GLU | MET | VAL | PRO | ILE |
| SEQRES | 9 | 459 | ILE | ALA | GLN | TYR | GLY | ASP | VAL | LEU | VAL | ARG | ASN | LEU | ARG |
| SEQRES | 10 | 459 | ARG | GLU | ALA | GLU | THR | GLY | LYS | PRO | VAL | THR | LEU | LYS | HIS |
| SEQRES | 11 | 459 | VAL | PHE | GLY | ALA | TYR | SER | MET | ASP | VAL | ILE | THR | SER | THR |
| SEQRES | 12 | 459 | SER | PHE | GLY | VAL | SER | ILE | ASP | SER | LEU | ASN | ASN | PRO | GLN |
| SEQRES | 13 | 459 | ASP | PRO | PHE | VAL | GLU | ASN | THR | LYS | LYS | LEU | LEU | ARG | PHE |
| SEQRES | 14 | 459 | ASN | PRO | LEU | ASP | PRO | PHE | VAL | LEU | SER | ILE | LYS | VAL | PHE |
| SEQRES | 15 | 459 | PRO | PHE | LEU | THR | PRO | ILE | LEU | GLU | ALA | LEU | ASN | ILE | THR |
| SEQRES | 16 | 459 | VAL | PHE | PRO | ARG | LYS | VAL | ILE | SER | PHE | LEU | THR | LYS | SER |
| SEQRES | 17 | 459 | VAL | LYS | GLN | ILE | LYS | GLU | GLY | ARG | LEU | LYS | GLU | THR | GLN |
| SEQRES | 18 | 459 | LYS | HIS | ARG | VAL | ASP | PHE | LEU | GLN | LEU | MET | ILE | ASP | SER |
| SEQRES | 19 | 459 | GLN | ASN | SER | LYS | ASP | SER | GLU | THR | HIS | LYS | ALA | LEU | SER |
| SEQRES | 20 | 459 | ASP | LEU | GLU | LEU | MET | ALA | GLN | SER | ILE | ILE | PHE | ILE | PHE |
| SEQRES | 21 | 459 | ALA | GLY | TYR | GLU | THR | THR | SER | SER | VAL | LEU | SER | PHE | ILE |
| SEQRES | 22 | 459 | ILE | TYR | GLU | LEU | ALA | THR | HIS | PRO | ASP | VAL | GLN | GLN | LYS |
| SEQRES | 23 | 459 | VAL | GLN | LYS | GLU | ILE | ASP | THR | VAL | LEU | PRO | ASN | LYS | ALA |
| SEQRES | 24 | 459 | PRO | PRO | THR | TYR | ASP | THR | VAL | LEU | GLN | LEU | GLU | TYR | LEU |
| SEQRES | 25 | 459 | ASP | MET | VAL | VAL | ASN | GLU | THR | LEU | ARG | LEU | PHE | PRO | VAL |
| SEQRES | 26 | 459 | ALA | MET | ARG | LEU | GLU | ARG | VAL | CYS | LYS | LYS | ASP | VAL | GLU |
| SEQRES | 27 | 459 | ILE | ASN | GLY | MET | PHE | ILE | PRO | LYS | GLY | VAL | VAL | VAL | MET |
| SEQRES | 28 | 459 | ILE | PRO | SER | TYR | VAL | LEU | HIS | HIS | ASP | PRO | LYS | TYR | TRP |
| SEQRES | 29 | 459 | THR | GLU | PRO | GLU | LYS | PHE | LEU | PRO | GLU | ARG | PHE | SER | LYS |
| SEQRES | 30 | 459 | LYS | ASN | LYS | ASP | ASN | ILE | ASP | PRO | TYR | ILE | TYR | THR | PRO |
| SEQRES | 31 | 459 | PHE | GLY | SER | GLY | PRO | ARG | ASN | CYS | ILE | GLY | MET | ARG | PHE |
| SEQRES | 32 | 459 | ALA | LEU | VAL | ASN | MET | LYS | LEU | ALA | LEU | VAL | ARG | VAL | LEU |
| SEQRES | 33 | 459 | GLN | ASN | PHE | SER | PHE | LYS | PRO | CYS | LYS | GLU | THR | GLN | ILE |
| SEQRES | 34 | 459 | PRO | LEU | LYS | LEU | ARG | PHE | GLY | GLY | LEU | LEU | LEU | THR | GLU |
| SEQRES | 35 | 459 | LYS | PRO | ILE | VAL | LEU | LYS | ALA | GLU | SER | ARG | ASP | GLU | THR |
| SEQRES | 36 | 459 | VAL | SER | GLY | ALA | | | | | | | | | |
| HET | HEM | 600 | | | | | | | | | | | | | |

| | | |
|---|---|---|
| HETNAM | HEM | HEME |
| HETSYN | HEM | 3,7,12,17-TETRAMETHYL-8,13-DIVINYL-2,18-PORPHINEDIPROPIONIC ACID |
| FORMUL | HEM | C34 H34 N4 O4 FE1 |

| | | | | | | | | | | |
|---|---|---|---|---|---|---|---|---|---|---|
| ATOM | 1 | N | PRO | 45 | 24.768 | 6.244 | −5.895 | 1.00 | 0.00 | 3A7 |
| ATOM | 2 | CD | PRO | 45 | 25.053 | 5.448 | −4.648 | 1.00 | 0.00 | 3A7 |
| ATOM | 3 | CA | PRO | 45 | 24.705 | 5.319 | −7.064 | 1.00 | 0.00 | 3A7 |
| ATOM | 4 | CB | PRO | 45 | 24.467 | 3.950 | −6.422 | 1.00 | 0.00 | 3A7 |
| ATOM | 5 | CG | PRO | 45 | 25.292 | 4.018 | −5.139 | 1.00 | 0.00 | 3A7 |
| ATOM | 6 | C | PRO | 45 | 23.594 | 5.801 | −7.950 | 1.00 | 0.00 | 3A7 |
| ATOM | 7 | O | PRO | 45 | 23.387 | 7.010 | −8.057 | 1.00 | 0.00 | 3A7 |
| ATOM | 8 | N | PHE | 46 | 22.870 | 4.857 | −8.600 | 1.00 | 0.00 | 3A7 |
| ATOM | 9 | CA | PHE | 46 | 21.751 | 5.140 | −9.472 | 1.00 | 0.00 | 3A7 |
| ATOM | 10 | CB | PHE | 46 | 21.853 | 4.416 | −10.835 | 1.00 | 0.00 | 3A7 |
| ATOM | 11 | CG | PHE | 46 | 23.083 | 4.887 | −11.553 | 1.00 | 0.00 | 3A7 |
| ATOM | 12 | CD1 | PHE | 46 | 24.173 | 4.033 | −11.730 | 1.00 | 0.00 | 3A7 |
| ATOM | 13 | CD2 | PHE | 46 | 23.155 | 6.189 | −12.049 | 1.00 | 0.00 | 3A7 |
| ATOM | 14 | CE1 | PHE | 46 | 25.317 | 4.474 | −12.390 | 1.00 | 0.00 | 3A7 |
| ATOM | 15 | CE2 | PHE | 46 | 24.297 | 6.633 | −12.709 | 1.00 | 0.00 | 3A7 |
| ATOM | 16 | CZ | PHE | 46 | 25.380 | 5.775 | −12.879 | 1.00 | 0.00 | 3A7 |
| ATOM | 17 | C | PHE | 46 | 20.491 | 4.691 | −8.782 | 1.00 | 0.00 | 3A7 |
| ATOM | 18 | O | PHE | 46 | 19.400 | 5.166 | −9.096 | 1.00 | 0.00 | 3A7 |
| ATOM | 19 | N | LEU | 47 | 20.629 | 3.749 | −7.814 | 1.00 | 0.00 | 3A7 |
| ATOM | 20 | CA | LEU | 47 | 19.541 | 3.204 | −7.035 | 1.00 | 0.00 | 3A7 |
| ATOM | 21 | CB | LEU | 47 | 19.661 | 1.667 | −6.874 | 1.00 | 0.00 | 3A7 |
| ATOM | 22 | CG | LEU | 47 | 18.490 | 0.978 | −6.129 | 1.00 | 0.00 | 3A7 |
| ATOM | 23 | CD1 | LEU | 47 | 17.133 | 1.202 | −6.826 | 1.00 | 0.00 | 3A7 |
| ATOM | 24 | CD2 | LEU | 47 | 18.768 | −0.526 | −5.942 | 1.00 | 0.00 | 3A7 |
| ATOM | 25 | C | LEU | 47 | 19.573 | 3.860 | −5.681 | 1.00 | 0.00 | 3A7 |
| ATOM | 26 | O | LEU | 47 | 20.619 | 3.930 | −5.036 | 1.00 | 0.00 | 3A7 |
| ATOM | 27 | N | GLY | 48 | 18.396 | 4.349 | −5.225 | 1.00 | 0.00 | 3A7 |
| ATOM | 28 | CA | GLY | 48 | 18.240 | 4.966 | −3.933 | 1.00 | 0.00 | 3A7 |
| ATOM | 29 | C | GLY | 48 | 16.930 | 4.491 | −3.395 | 1.00 | 0.00 | 3A7 |
| ATOM | 30 | O | GLY | 48 | 15.938 | 5.218 | −3.414 | 1.00 | 0.00 | 3A7 |
| ATOM | 31 | N | ASN | 49 | 16.911 | 3.229 | −2.898 | 1.00 | 0.00 | 3A7 |
| ATOM | 32 | CA | ASN | 49 | 15.741 | 2.585 | −2.348 | 1.00 | 0.00 | 3A7 |
| ATOM | 33 | CB | ASN | 49 | 15.445 | 1.235 | −3.059 | 1.00 | 0.00 | 3A7 |
| ATOM | 34 | CG | ASN | 49 | 14.046 | 0.705 | −2.706 | 1.00 | 0.00 | 3A7 |
| ATOM | 35 | OD1 | ASN | 49 | 13.035 | 1.349 | −3.011 | 1.00 | 0.00 | 3A7 |
| ATOM | 36 | ND2 | ASN | 49 | 14.005 | −0.494 | −2.051 | 1.00 | 0.00 | 3A7 |
| ATOM | 37 | C | ASN | 49 | 16.016 | 2.375 | −0.880 | 1.00 | 0.00 | 3A7 |
| ATOM | 38 | O | ASN | 49 | 17.169 | 2.278 | −0.463 | 1.00 | 0.00 | 3A7 |
| ATOM | 39 | N | ALA | 50 | 14.933 | 2.293 | −0.065 | 1.00 | 0.00 | 3A7 |
| ATOM | 40 | CA | ALA | 50 | 14.998 | 2.111 | 1.369 | 1.00 | 0.00 | 3A7 |
| ATOM | 41 | CB | ALA | 50 | 13.847 | 2.834 | 2.098 | 1.00 | 0.00 | 3A7 |
| ATOM | 42 | C | ALA | 50 | 14.941 | 0.641 | 1.697 | 1.00 | 0.00 | 3A7 |
| ATOM | 43 | O | ALA | 50 | 13.866 | 0.049 | 1.787 | 1.00 | 0.00 | 3A7 |
| ATOM | 44 | N | LEU | 51 | 16.133 | 0.032 | 1.885 | 1.00 | 0.00 | 3A7 |
| ATOM | 45 | CA | LEU | 51 | 16.271 | −1.360 | 2.232 | 1.00 | 0.00 | 3A7 |
| ATOM | 46 | CB | LEU | 51 | 16.271 | −2.314 | 1.001 | 1.00 | 0.00 | 3A7 |
| ATOM | 47 | CG | LEU | 51 | 17.055 | −1.862 | −0.262 | 1.00 | 0.00 | 3A7 |
| ATOM | 48 | CD1 | LEU | 51 | 18.587 | −1.811 | −0.098 | 1.00 | 0.00 | 3A7 |
| ATOM | 49 | CD2 | LEU | 51 | 16.690 | −2.765 | −1.456 | 1.00 | 0.00 | 3A7 |
| ATOM | 50 | C | LEU | 51 | 17.550 | −1.468 | 3.012 | 1.00 | 0.00 | 3A7 |
| ATOM | 51 | O | LEU | 51 | 18.327 | −0.517 | 3.085 | 1.00 | 0.00 | 3A7 |
| ATOM | 52 | N | SER | 52 | 17.794 | −2.657 | 3.613 | 1.00 | 0.00 | 3A7 |
| ATOM | 53 | CA | SER | 52 | 19.005 | −2.956 | 4.343 | 1.00 | 0.00 | 3A7 |
| ATOM | 54 | CB | SER | 52 | 18.741 | −3.361 | 5.821 | 1.00 | 0.00 | 3A7 |
| ATOM | 55 | OG | SER | 52 | 17.737 | −4.363 | 5.945 | 1.00 | 0.00 | 3A7 |
| ATOM | 56 | C | SER | 52 | 19.734 | −4.023 | 3.562 | 1.00 | 0.00 | 3A7 |
| ATOM | 57 | O | SER | 52 | 20.164 | −3.782 | 2.435 | 1.00 | 0.00 | 3A7 |
| ATOM | 58 | N | PHE | 53 | 19.898 | −5.231 | 4.155 | 1.00 | 0.00 | 3A7 |
| ATOM | 59 | CA | PHE | 53 | 20.599 | −6.341 | 3.550 | 1.00 | 0.00 | 3A7 |
| ATOM | 60 | CB | PHE | 53 | 21.908 | −6.703 | 4.297 | 1.00 | 0.00 | 3A7 |
| ATOM | 61 | CG | PHE | 53 | 22.730 | −5.456 | 4.439 | 1.00 | 0.00 | 3A7 |
| ATOM | 62 | CD1 | PHE | 53 | 22.819 | −4.809 | 5.673 | 1.00 | 0.00 | 3A7 |
| ATOM | 63 | CD2 | PHE | 53 | 23.346 | −4.880 | 3.327 | 1.00 | 0.00 | 3A7 |
| ATOM | 64 | CE1 | PHE | 53 | 23.491 | −3.598 | 5.793 | 1.00 | 0.00 | 3A7 |
| ATOM | 65 | CE2 | PHE | 53 | 24.018 | −3.667 | 3.446 | 1.00 | 0.00 | 3A7 |
| ATOM | 66 | CZ | PHE | 53 | 24.085 | −3.022 | 4.675 | 1.00 | 0.00 | 3A7 |
| ATOM | 67 | C | PHE | 53 | 19.639 | −7.494 | 3.594 | 1.00 | 0.00 | 3A7 |
| ATOM | 68 | O | PHE | 53 | 19.885 | −8.495 | 4.264 | 1.00 | 0.00 | 3A7 |
| ATOM | 69 | N | ARG | 54 | 18.491 | −7.330 | 2.875 | 1.00 | 0.00 | 3A7 |
| ATOM | 70 | CA | ARG | 54 | 17.327 | −8.205 | 2.828 | 1.00 | 0.00 | 3A7 |
| ATOM | 71 | CB | ARG | 54 | 17.629 | −9.719 | 2.641 | 1.00 | 0.00 | 3A7 |
| ATOM | 72 | CG | ARG | 54 | 18.536 | −10.045 | 1.437 | 1.00 | 0.00 | 3A7 |
| ATOM | 73 | CD | ARG | 54 | 17.996 | −9.573 | 0.079 | 1.00 | 0.00 | 3A7 |
| ATOM | 74 | NE | ARG | 54 | 19.049 | −9.836 | −0.962 | 1.00 | 0.00 | 3A7 |
| ATOM | 75 | CZ | ARG | 54 | 19.036 | −10.925 | −1.791 | 1.00 | 0.00 | 3A7 |
| ATOM | 76 | NH1 | ARG | 54 | 20.065 | −11.113 | −2.669 | 1.00 | 0.00 | 3A7 |
| ATOM | 77 | NH2 | ARG | 54 | 18.013 | −11.826 | −1.753 | 1.00 | 0.00 | 3A7 |
| ATOM | 78 | C | ARG | 54 | 16.514 | −7.983 | 4.087 | 1.00 | 0.00 | 3A7 |
| ATOM | 79 | O | ARG | 54 | 16.760 | −8.611 | 5.116 | 1.00 | 0.00 | 3A7 |
| ATOM | 80 | N | LYS | 55 | 15.545 | −7.030 | 4.028 | 1.00 | 0.00 | 3A7 |
| ATOM | 81 | CA | LYS | 55 | 14.927 | −6.447 | 5.205 | 1.00 | 0.00 | 3A7 |
| ATOM | 82 | CB | LYS | 55 | 14.520 | −4.971 | 5.023 | 1.00 | 0.00 | 3A7 |
| ATOM | 83 | CG | LYS | 55 | 13.327 | −4.656 | 4.092 | 1.00 | 0.00 | 3A7 |
| ATOM | 84 | CD | LYS | 55 | 13.574 | −4.842 | 2.582 | 1.00 | 0.00 | 3A7 |
| ATOM | 85 | CE | LYS | 55 | 13.074 | −6.171 | 1.993 | 1.00 | 0.00 | 3A7 |
| ATOM | 86 | NZ | LYS | 55 | 11.612 | −6.322 | 2.178 | 1.00 | 0.00 | 3A7 |
| ATOM | 87 | C | LYS | 55 | 13.767 | −7.241 | 5.777 | 1.00 | 0.00 | 3A7 |
| ATOM | 88 | O | LYS | 55 | 13.403 | −7.067 | 6.938 | 1.00 | 0.00 | 3A7 |
| ATOM | 89 | N | GLY | 56 | 13.158 | −8.146 | 4.991 | 1.00 | 0.00 | 3A7 |
| ATOM | 90 | CA | GLY | 56 | 12.066 | −8.950 | 5.491 | 1.00 | 0.00 | 3A7 |
| ATOM | 91 | C | GLY | 56 | 11.995 | −10.146 | 4.604 | 1.00 | 0.00 | 3A7 |
| ATOM | 92 | O | GLY | 56 | 11.061 | −10.334 | 3.823 | 1.00 | 0.00 | 3A7 |
| ATOM | 93 | N | TYR | 57 | 13.009 | −11.031 | 4.730 | 1.00 | 0.00 | 3A7 |
| ATOM | 94 | CA | TYR | 57 | 13.171 | −12.222 | 3.922 | 1.00 | 0.00 | 3A7 |
| ATOM | 95 | CB | TYR | 57 | 14.496 | −12.913 | 4.319 | 1.00 | 0.00 | 3A7 |
| ATOM | 96 | CG | TYR | 57 | 15.183 | −13.558 | 3.146 | 1.00 | 0.00 | 3A7 |
| ATOM | 97 | CD1 | TYR | 57 | 15.496 | −12.832 | 1.995 | 1.00 | 0.00 | 3A7 |
| ATOM | 98 | CD2 | TYR | 57 | 15.566 | −14.898 | 3.223 | 1.00 | 0.00 | 3A7 |
| ATOM | 99 | CE1 | TYR | 57 | 16.160 | −13.440 | 0.934 | 1.00 | 0.00 | 3A7 |
| ATOM | 100 | CE2 | TYR | 57 | 16.240 | −15.506 | 2.168 | 1.00 | 0.00 | 3A7 |
| ATOM | 101 | CZ | TYR | 57 | 16.538 | −14.777 | 1.021 | 1.00 | 0.00 | 3A7 |
| ATOM | 102 | OH | TYR | 57 | 17.229 | −15.392 | −0.043 | 1.00 | 0.00 | 3A7 |
| ATOM | 103 | C | TYR | 57 | 12.013 | −13.191 | 4.022 | 1.00 | 0.00 | 3A7 |
| ATOM | 104 | O | TYR | 57 | 11.631 | −13.781 | 3.021 | 1.00 | 0.00 | 3A7 |
| ATOM | 105 | N | TRP | 58 | 11.379 | −13.353 | 5.211 | 1.00 | 0.00 | 3A7 |
| ATOM | 106 | CA | TRP | 58 | 10.261 | −14.269 | 5.359 | 1.00 | 0.00 | 3A7 |
| ATOM | 107 | CB | TRP | 58 | 9.872 | −14.515 | 6.832 | 1.00 | 0.00 | 3A7 |
| ATOM | 108 | CG | TRP | 58 | 9.333 | −13.318 | 7.606 | 1.00 | 0.00 | 3A7 |
| ATOM | 109 | CD2 | TRP | 58 | 8.006 | −13.247 | 8.157 | 1.00 | 0.00 | 3A7 |
| ATOM | 110 | CD1 | TRP | 58 | 9.925 | −12.115 | 7.871 | 1.00 | 0.00 | 3A7 |
| ATOM | 111 | NE1 | TRP | 58 | 9.051 | −11.297 | 8.548 | 1.00 | 0.00 | 3A7 |
| ATOM | 112 | CE2 | TRP | 58 | 7.869 | −11.978 | 8.741 | 1.00 | 0.00 | 3A7 |
| ATOM | 113 | CE3 | TRP | 58 | 6.969 | −14.158 | 8.175 | 1.00 | 0.00 | 3A7 |
| ATOM | 114 | CZ2 | TRP | 58 | 6.694 | −11.604 | 9.360 | 1.00 | 0.00 | 3A7 |
| ATOM | 115 | CZ3 | TRP | 58 | 5.782 | −13.780 | 8.796 | 1.00 | 0.00 | 3A7 |
| ATOM | 116 | CH2 | TRP | 58 | 5.647 | −12.522 | 9.382 | 1.00 | 0.00 | 3A7 |
| ATOM | 117 | C | TRP | 58 | 9.036 | −13.824 | 4.612 | 1.00 | 0.00 | 3A7 |
| ATOM | 118 | O | TRP | 58 | 8.302 | −14.664 | 4.106 | 1.00 | 0.00 | 3A7 |
| ATOM | 119 | N | THR | 59 | 8.780 | −12.490 | 4.502 | 1.00 | 0.00 | 3A7 |
| ATOM | 120 | CA | THR | 59 | 7.645 | −11.989 | 3.737 | 1.00 | 0.00 | 3A7 |
| ATOM | 121 | CB | THR | 59 | 7.213 | −10.582 | 4.136 | 1.00 | 0.00 | 3A7 |
| ATOM | 122 | OG1 | THR | 59 | 8.282 | −9.643 | 4.073 | 1.00 | 0.00 | 3A7 |
| ATOM | 123 | CG2 | THR | 59 | 6.673 | −10.634 | 5.579 | 1.00 | 0.00 | 3A7 |
| ATOM | 124 | C | THR | 59 | 7.925 | −12.067 | 2.267 | 1.00 | 0.00 | 3A7 |
| ATOM | 125 | O | THR | 59 | 7.011 | −12.232 | 1.471 | 1.00 | 0.00 | 3A7 |
| ATOM | 126 | N | PHE | 60 | 9.218 | −12.018 | 1.884 | 1.00 | 0.00 | 3A7 |
| ATOM | 127 | CA | PHE | 60 | 9.659 | −12.244 | 0.530 | 1.00 | 0.00 | 3A7 |
| ATOM | 128 | CB | PHE | 60 | 11.132 | −11.803 | 0.330 | 1.00 | 0.00 | 3A7 |
| ATOM | 129 | CG | PHE | 60 | 11.512 | −11.735 | −1.126 | 1.00 | 0.00 | 3A7 |
| ATOM | 130 | CD1 | PHE | 60 | 10.889 | −10.822 | −1.978 | 1.00 | 0.00 | 3A7 |
| ATOM | 131 | CD2 | PHE | 60 | 12.481 | −12.595 | −1.647 | 1.00 | 0.00 | 3A7 |
| ATOM | 132 | CE1 | PHE | 60 | 11.222 | −10.772 | −3.328 | 1.00 | 0.00 | 3A7 |
| ATOM | 133 | CE2 | PHE | 60 | 12.816 | −12.548 | −2.997 | 1.00 | 0.00 | 3A7 |
| ATOM | 134 | CZ | PHE | 60 | 12.186 | −11.636 | −3.839 | 1.00 | 0.00 | 3A7 |
| ATOM | 135 | C | PHE | 60 | 9.505 | −13.695 | 0.122 | 1.00 | 0.00 | 3A7 |
| ATOM | 136 | O | PHE | 60 | 8.967 | −14.011 | −0.936 | 1.00 | 0.00 | 3A7 |
| ATOM | 137 | N | ASP | 61 | 9.940 | −14.632 | 0.991 | 1.00 | 0.00 | 3A7 |
| ATOM | 138 | CA | ASP | 61 | 9.855 | −16.051 | 0.737 | 1.00 | 0.00 | 3A7 |
| ATOM | 139 | CB | ASP | 61 | 10.621 | −16.892 | 1.774 | 1.00 | 0.00 | 3A7 |
| ATOM | 140 | CG | ASP | 61 | 12.134 | −16.729 | 1.612 | 1.00 | 0.00 | 3A7 |
| ATOM | 141 | OD1 | ASP | 61 | 12.612 | −16.736 | 0.447 | 1.00 | 0.00 | 3A7 |
| ATOM | 142 | OD2 | ASP | 61 | 12.818 | −16.555 | 2.655 | 1.00 | 0.00 | 3A7 |
| ATOM | 143 | C | ASP | 61 | 8.415 | −16.503 | 0.698 | 1.00 | 0.00 | 3A7 |
| ATOM | 144 | O | ASP | 61 | 8.070 | −17.371 | −0.085 | 1.00 | 0.00 | 3A7 |
| ATOM | 145 | N | MET | 62 | 7.521 | −15.870 | 1.491 | 1.00 | 0.00 | 3A7 |
| ATOM | 146 | CA | MET | 62 | 6.098 | −16.134 | 1.452 | 1.00 | 0.00 | 3A7 |
| ATOM | 147 | CB | MET | 62 | 5.374 | −15.468 | 2.623 | 1.00 | 0.00 | 3A7 |
| ATOM | 148 | CG | MET | 62 | 5.596 | −16.224 | 3.947 | 1.00 | 0.00 | 3A7 |
| ATOM | 149 | SD | MET | 62 | 4.969 | −15.359 | 5.422 | 1.00 | 0.00 | 3A7 |
| ATOM | 150 | CE | MET | 62 | 3.199 | −15.481 | 5.031 | 1.00 | 0.00 | 3A7 |
| ATOM | 151 | C | MET | 62 | 5.450 | −15.672 | 0.168 | 1.00 | 0.00 | 3A7 |
| ATOM | 152 | O | MET | 62 | 4.555 | −16.335 | −0.342 | 1.00 | 0.00 | 3A7 |
| ATOM | 153 | N | GLU | 63 | 5.922 | −14.555 | −0.430 | 1.00 | 0.00 | 3A7 |
| ATOM | 154 | CA | GLU | 63 | 5.457 | −14.094 | −1.722 | 1.00 | 0.00 | 3A7 |
| ATOM | 155 | CB | GLU | 63 | 5.987 | −12.683 | −2.039 | 1.00 | 0.00 | 3A7 |
| ATOM | 156 | CG | GLU | 63 | 5.290 | −11.593 | −1.206 | 1.00 | 0.00 | 3A7 |
| ATOM | 157 | CD | GLU | 63 | 5.961 | −10.239 | −1.427 | 1.00 | 0.00 | 3A7 |
| ATOM | 158 | OE1 | GLU | 63 | 6.943 | −10.169 | −2.214 | 1.00 | 0.00 | 3A7 |
| ATOM | 159 | OE2 | GLU | 63 | 5.495 | −9.249 | −0.802 | 1.00 | 0.00 | 3A7 |
| ATOM | 160 | C | GLU | 63 | 5.865 | −15.044 | −2.831 | 1.00 | 0.00 | 3A7 |
| ATOM | 161 | O | GLU | 63 | 5.087 | −15.339 | −3.734 | 1.00 | 0.00 | 3A7 |
| ATOM | 162 | N | CYS | 64 | 7.093 | −15.606 | −2.747 | 1.00 | 0.00 | 3A7 |
| ATOM | 163 | CA | CYS | 64 | 7.601 | −16.598 | −3.674 | 1.00 | 0.00 | 3A7 |
| ATOM | 164 | CB | CYS | 64 | 9.045 | −16.969 | −3.362 | 1.00 | 0.00 | 3A7 |
| ATOM | 165 | SG | CYS | 64 | 9.973 | −15.524 | −3.736 | 1.00 | 0.00 | 3A7 |
| ATOM | 166 | C | CYS | 64 | 6.805 | −17.853 | −3.631 | 1.00 | 0.00 | 3A7 |
| ATOM | 167 | O | CYS | 64 | 6.413 | −18.402 | −4.655 | 1.00 | 0.00 | 3A7 |
| ATOM | 168 | N | TYR | 65 | 6.477 | −18.323 | −2.413 | 1.00 | 0.00 | 3A7 |
| ATOM | 169 | CA | TYR | 65 | 5.695 | −19.526 | −2.251 | 1.00 | 0.00 | 3A7 |
| ATOM | 170 | CB | TYR | 65 | 5.638 | −19.993 | −0.802 | 1.00 | 0.00 | 3A7 |
| ATOM | 171 | CG | TYR | 65 | 7.043 | −20.246 | −0.379 | 1.00 | 0.00 | 3A7 |
| ATOM | 172 | CD1 | TYR | 65 | 7.458 | −19.725 | 0.834 | 1.00 | 0.00 | 3A7 |
| ATOM | 173 | CD2 | TYR | 65 | 7.985 | −20.850 | −1.222 | 1.00 | 0.00 | 3A7 |
| ATOM | 174 | CE1 | TYR | 65 | 8.800 | −19.750 | 1.182 | 1.00 | 0.00 | 3A7 |
| ATOM | 175 | CE2 | TYR | 65 | 9.336 | −20.816 | −0.901 | 1.00 | 0.00 | 3A7 |
| ATOM | 176 | CZ | TYR | 65 | 9.743 | −20.216 | 0.283 | 1.00 | 0.00 | 3A7 |
| ATOM | 177 | OH | TYR | 65 | 11.101 | −20.003 | 0.572 | 1.00 | 0.00 | 3A7 |
| ATOM | 178 | C | TYR | 65 | 4.275 | −19.373 | −2.701 | 1.00 | 0.00 | 3A7 |
| ATOM | 179 | O | TYR | 65 | 3.706 | −20.319 | −3.228 | 1.00 | 0.00 | 3A7 |
| ATOM | 180 | N | LYS | 66 | 3.656 | −18.174 | −2.556 | 1.00 | 0.00 | 3A7 |
| ATOM | 181 | CA | LYS | 66 | 2.313 | −17.922 | −3.043 | 1.00 | 0.00 | 3A7 |
| ATOM | 182 | CB | LYS | 66 | 1.810 | −16.564 | −2.544 | 1.00 | 0.00 | 3A7 |
| ATOM | 183 | CG | LYS | 66 | 1.450 | −16.579 | −1.049 | 1.00 | 0.00 | 3A7 |
| ATOM | 184 | CD | LYS | 66 | 1.162 | −15.185 | −0.470 | 1.00 | 0.00 | 3A7 |
| ATOM | 185 | CE | LYS | 66 | −0.063 | −14.502 | −1.089 | 1.00 | 0.00 | 3A7 |
| ATOM | 186 | NZ | LYS | 66 | −0.299 | −13.182 | −0.461 | 1.00 | 0.00 | 3A7 |
| ATOM | 187 | C | LYS | 66 | 2.244 | −17.957 | −4.556 | 1.00 | 0.00 | 3A7 |
| ATOM | 188 | O | LYS | 66 | 1.227 | −18.326 | −5.129 | 1.00 | 0.00 | 3A7 |
| ATOM | 189 | N | LYS | 67 | 3.349 | −17.595 | −5.243 | 1.00 | 0.00 | 3A7 |
| ATOM | 190 | CA | LYS | 67 | 3.417 | −17.539 | −6.682 | 1.00 | 0.00 | 3A7 |
| ATOM | 191 | CB | LYS | 67 | 4.377 | −16.414 | −7.137 | 1.00 | 0.00 | 3A7 |
| ATOM | 192 | CG | LYS | 67 | 4.368 | −16.145 | −8.651 | 1.00 | 0.00 | 3A7 |
| ATOM | 193 | CD | LYS | 67 | 5.148 | −14.878 | −9.034 | 1.00 | 0.00 | 3A7 |
| ATOM | 194 | CE | LYS | 67 | 5.201 | −14.629 | −10.547 | 1.00 | 0.00 | 3A7 |
| ATOM | 195 | NZ | LYS | 67 | 3.843 | −14.424 | −11.100 | 1.00 | 0.00 | 3A7 |
| ATOM | 196 | C | LYS | 67 | 3.853 | −18.855 | −7.284 | 1.00 | 0.00 | 3A7 |
| ATOM | 197 | O | LYS | 67 | 3.254 | −19.336 | −8.244 | 1.00 | 0.00 | 3A7 |
| ATOM | 198 | N | TYR | 68 | 4.942 | −19.451 | −6.748 | 1.00 | 0.00 | 3A7 |
| ATOM | 199 | CA | TYR | 68 | 5.626 | −20.573 | −7.355 | 1.00 | 0.00 | 3A7 |
| ATOM | 200 | CB | TYR | 68 | 7.148 | −20.402 | −7.314 | 1.00 | 0.00 | 3A7 |
| ATOM | 201 | CG | TYR | 68 | 7.522 | −19.259 | −8.216 | 1.00 | 0.00 | 3A7 |
| ATOM | 202 | CD1 | TYR | 68 | 7.789 | −17.989 | −7.702 | 1.00 | 0.00 | 3A7 |
| ATOM | 203 | CD2 | TYR | 68 | 7.593 | −19.457 | −9.595 | 1.00 | 0.00 | 3A7 |
| ATOM | 204 | CE1 | TYR | 68 | 8.122 | −16.937 | −8.550 | 1.00 | 0.00 | 3A7 |
| ATOM | 205 | CE2 | TYR | 68 | 7.927 | −18.408 | −10.447 | 1.00 | 0.00 | 3A7 |
| ATOM | 206 | CZ | TYR | 68 | 8.193 | −17.146 | −9.924 | 1.00 | 0.00 | 3A7 |
| ATOM | 207 | OH | TYR | 68 | 8.537 | −16.080 | −10.784 | 1.00 | 0.00 | 3A7 |
| ATOM | 208 | C | TYR | 68 | 5.236 | −21.899 | −6.770 | 1.00 | 0.00 | 3A7 |
| ATOM | 209 | O | TYR | 68 | 5.299 | −22.918 | −7.453 | 1.00 | 0.00 | 3A7 |
| ATOM | 210 | N | ARG | 69 | 4.785 | −21.912 | −5.500 | 1.00 | 0.00 | 3A7 |
| ATOM | 211 | CA | ARG | 69 | 4.142 | −23.040 | −4.856 | 1.00 | 0.00 | 3A7 |
| ATOM | 212 | CB | ARG | 69 | 3.046 | −23.733 | −5.717 | 1.00 | 0.00 | 3A7 |
| ATOM | 213 | CG | ARG | 69 | 1.942 | −22.762 | −6.180 | 1.00 | 0.00 | 3A7 |
| ATOM | 214 | CD | ARG | 69 | 0.844 | −23.441 | −7.009 | 1.00 | 0.00 | 3A7 |
| ATOM | 215 | NE | ARG | 69 | 1.454 | −23.933 | −8.287 | 1.00 | 0.00 | 3A7 |
| ATOM | 216 | CZ | ARG | 69 | 0.741 | −24.664 | −9.197 | 1.00 | 0.00 | 3A7 |
| ATOM | 217 | NH1 | ARG | 69 | 1.345 | −25.083 | −10.347 | 1.00 | 0.00 | 3A7 |
| ATOM | 218 | NH2 | ARG | 69 | −0.565 | −24.981 | −8.963 | 1.00 | 0.00 | 3A7 |
| ATOM | 219 | C | ARG | 69 | 5.132 | −23.968 | −4.257 | 1.00 | 0.00 | 3A7 |
| ATOM | 220 | O | ARG | 69 | 6.140 | −23.556 | −3.663 | 1.00 | 0.00 | 3A7 |
| ATOM | 221 | N | LYS | 70 | 4.815 | −25.251 | −4.585 | 1.00 | 0.00 | 3A7 |
| ATOM | 222 | CA | LYS | 70 | 5.551 | −26.459 | −4.410 | 1.00 | 0.00 | 3A7 |
| ATOM | 223 | CB | LYS | 70 | 4.828 | −27.726 | −4.955 | 1.00 | 0.00 | 3A7 |
| ATOM | 224 | CG | LYS | 70 | 3.527 | −28.142 | −4.233 | 1.00 | 0.00 | 3A7 |
| ATOM | 225 | CD | LYS | 70 | 2.245 | −27.345 | −4.536 | 1.00 | 0.00 | 3A7 |
| ATOM | 226 | CE | LYS | 70 | 1.635 | −27.577 | −5.929 | 1.00 | 0.00 | 3A7 |
| ATOM | 227 | NZ | LYS | 70 | 2.449 | −26.976 | −7.009 | 1.00 | 0.00 | 3A7 |
| ATOM | 228 | C | LYS | 70 | 6.866 | −26.352 | −5.122 | 1.00 | 0.00 | 3A7 |
| ATOM | 229 | O | LYS | 70 | 7.015 | −26.860 | −6.233 | 1.00 | 0.00 | 3A7 |
| ATOM | 230 | N | VAL | 71 | 7.834 | −25.662 | −4.449 | 1.00 | 0.00 | 3A7 |
| ATOM | 231 | CA | VAL | 71 | 9.235 | −25.641 | −4.770 | 1.00 | 0.00 | 3A7 |
| ATOM | 232 | CB | VAL | 71 | 9.760 | −26.967 | −5.362 | 1.00 | 0.00 | 3A7 |
| ATOM | 233 | CG1 | VAL | 71 | 11.270 | −26.882 | −5.635 | 1.00 | 0.00 | 3A7 |
| ATOM | 234 | CG2 | VAL | 71 | 9.523 | −28.158 | −4.411 | 1.00 | 0.00 | 3A7 |
| ATOM | 235 | C | VAL | 71 | 9.454 | −24.472 | −5.708 | 1.00 | 0.00 | 3A7 |
| ATOM | 236 | O | VAL | 71 | 8.810 | −24.376 | −6.751 | 1.00 | 0.00 | 3A7 |
| ATOM | 237 | N | TRP | 72 | 10.373 | −23.545 | −5.337 | 1.00 | 0.00 | 3A7 |
| ATOM | 238 | CA | TRP | 72 | 10.743 | −22.433 | −6.182 | 1.00 | 0.00 | 3A7 |
| ATOM | 239 | CB | TRP | 72 | 10.009 | −21.089 | −5.839 | 1.00 | 0.00 | 3A7 |
| ATOM | 240 | CG | TRP | 72 | 10.772 | −19.873 | −5.306 | 1.00 | 0.00 | 3A7 |
| ATOM | 241 | CD2 | TRP | 72 | 11.148 | −18.737 | −6.108 | 1.00 | 0.00 | 3A7 |
| ATOM | 242 | CD1 | TRP | 72 | 11.256 | −19.633 | −4.054 | 1.00 | 0.00 | 3A7 |
| ATOM | 243 | NE1 | TRP | 72 | 11.935 | −18.438 | −4.024 | 1.00 | 0.00 | 3A7 |
| ATOM | 244 | CE2 | TRP | 72 | 11.871 | −17.866 | −5.277 | 1.00 | 0.00 | 3A7 |
| ATOM | 245 | CE3 | TRP | 72 | 10.923 | −18.434 | −7.436 | 1.00 | 0.00 | 3A7 |
| ATOM | 246 | CZ2 | TRP | 72 | 12.368 | −16.672 | −5.760 | 1.00 | 0.00 | 3A7 |
| ATOM | 247 | CZ3 | TRP | 72 | 11.414 | −17.223 | −7.920 | 1.00 | 0.00 | 3A7 |
| ATOM | 248 | CH2 | TRP | 72 | 12.124 | −16.354 | −7.094 | 1.00 | 0.00 | 3A7 |
| ATOM | 249 | C | TRP | 72 | 12.229 | −22.293 | −6.064 | 1.00 | 0.00 | 3A7 |
| ATOM | 250 | O | TRP | 72 | 12.824 | −22.782 | −5.112 | 1.00 | 0.00 | 3A7 |
| ATOM | 251 | N | GLY | 73 | 12.861 | −21.582 | −7.022 | 1.00 | 0.00 | 3A7 |
| ATOM | 252 | CA | GLY | 73 | 14.278 | −21.323 | −6.985 | 1.00 | 0.00 | 3A7 |
| ATOM | 253 | C | GLY | 73 | 14.511 | −19.850 | −6.821 | 1.00 | 0.00 | 3A7 |
| ATOM | 254 | O | GLY | 73 | 13.812 | −19.031 | −7.409 | 1.00 | 0.00 | 3A7 |
| ATOM | 255 | N | ILE | 74 | 15.530 | −19.484 | −6.009 | 1.00 | 0.00 | 3A7 |
| ATOM | 256 | CA | ILE | 74 | 15.929 | −18.120 | −5.754 | 1.00 | 0.00 | 3A7 |
| ATOM | 257 | CB | ILE | 74 | 15.600 | −17.676 | −4.328 | 1.00 | 0.00 | 3A7 |
| ATOM | 258 | CG2 | ILE | 74 | 16.301 | −18.543 | −3.258 | 1.00 | 0.00 | 3A7 |
| ATOM | 259 | CG1 | ILE | 74 | 15.835 | −16.159 | −4.124 | 1.00 | 0.00 | 3A7 |
| ATOM | 260 | CD | ILE | 74 | 15.205 | −15.621 | −2.836 | 1.00 | 0.00 | 3A7 |
| ATOM | 261 | C | ILE | 74 | 17.407 | −18.077 | −6.023 | 1.00 | 0.00 | 3A7 |
| ATOM | 262 | O | ILE | 74 | 18.100 | −19.082 | −5.904 | 1.00 | 0.00 | 3A7 |
| ATOM | 263 | N | TYR | 75 | 17.928 | −16.900 | −6.425 | 1.00 | 0.00 | 3A7 |
| ATOM | 264 | CA | TYR | 75 | 19.303 | −16.765 | −6.827 | 1.00 | 0.00 | 3A7 |
| ATOM | 265 | CB | TYR | 75 | 19.424 | −16.348 | −8.308 | 1.00 | 0.00 | 3A7 |
| ATOM | 266 | CG | TYR | 75 | 18.811 | −17.400 | −9.184 | 1.00 | 0.00 | 3A7 |
| ATOM | 267 | CD1 | TYR | 75 | 17.487 | −17.281 | −9.613 | 1.00 | 0.00 | 3A7 |
| ATOM | 268 | CD2 | TYR | 75 | 19.554 | −18.513 | −9.582 | 1.00 | 0.00 | 3A7 |
| ATOM | 269 | CE1 | TYR | 75 | 16.911 | −18.261 | −10.415 | 1.00 | 0.00 | 3A7 |
| ATOM | 270 | CE2 | TYR | 75 | 18.983 | −19.489 | −10.394 | 1.00 | 0.00 | 3A7 |
| ATOM | 271 | CZ | TYR | 75 | 17.660 | −19.367 | −10.807 | 1.00 | 0.00 | 3A7 |
| ATOM | 272 | OH | TYR | 75 | 17.079 | −20.361 | −11.624 | 1.00 | 0.00 | 3A7 |
| ATOM | 273 | C | TYR | 75 | 19.927 | −15.716 | −5.954 | 1.00 | 0.00 | 3A7 |
| ATOM | 274 | O | TYR | 75 | 19.589 | −14.543 | −6.059 | 1.00 | 0.00 | 3A7 |
| ATOM | 275 | N | ASP | 76 | 20.867 | −16.106 | −5.059 | 1.00 | 0.00 | 3A7 |
| ATOM | 276 | CA | ASP | 76 | 21.559 | −15.171 | −4.194 | 1.00 | 0.00 | 3A7 |
| ATOM | 277 | CB | ASP | 76 | 21.470 | −15.513 | −2.685 | 1.00 | 0.00 | 3A7 |
| ATOM | 278 | CG | ASP | 76 | 20.053 | −15.259 | −2.172 | 1.00 | 0.00 | 3A7 |
| ATOM | 279 | OD1 | ASP | 76 | 19.105 | −15.935 | −2.652 | 1.00 | 0.00 | 3A7 |
| ATOM | 280 | OD2 | ASP | 76 | 19.905 | −14.382 | −1.279 | 1.00 | 0.00 | 3A7 |
| ATOM | 281 | C | ASP | 76 | 23.004 | −15.175 | −4.597 | 1.00 | 0.00 | 3A7 |
| ATOM | 282 | O | ASP | 76 | 23.790 | −16.000 | −4.149 | 1.00 | 0.00 | 3A7 |
| ATOM | 283 | N | CYS | 77 | 23.393 | −14.210 | −5.459 | 1.00 | 0.00 | 3A7 |
| ATOM | 284 | CA | CYS | 77 | 24.738 | −13.896 | −5.902 | 1.00 | 0.00 | 3A7 |
| ATOM | 285 | CB | CYS | 77 | 25.569 | −13.074 | −4.858 | 1.00 | 0.00 | 3A7 |
| ATOM | 286 | SG | CYS | 77 | 25.906 | −13.854 | −3.237 | 1.00 | 0.00 | 3A7 |
| ATOM | 287 | C | CYS | 77 | 25.544 | −15.056 | −6.442 | 1.00 | 0.00 | 3A7 |
| ATOM | 288 | O | CYS | 77 | 26.561 | −15.442 | −5.879 | 1.00 | 0.00 | 3A7 |
| ATOM | 289 | N | GLN | 78 | 25.091 | −15.613 | −7.583 | 1.00 | 0.00 | 3A7 |
| ATOM | 290 | CA | GLN | 78 | 25.770 | −16.622 | −8.369 | 1.00 | 0.00 | 3A7 |
| ATOM | 291 | CB | GLN | 78 | 27.325 | −16.549 | −8.440 | 1.00 | 0.00 | 3A7 |
| ATOM | 292 | CG | GLN | 78 | 27.848 | −15.222 | −9.017 | 1.00 | 0.00 | 3A7 |
| ATOM | 293 | CD | GLN | 78 | 29.378 | −15.274 | −9.063 | 1.00 | 0.00 | 3A7 |
| ATOM | 294 | OE1 | GLN | 78 | 29.961 | −16.057 | −9.823 | 1.00 | 0.00 | 3A7 |
| ATOM | 295 | NE2 | GLN | 78 | 30.031 | −14.415 | −8.222 | 1.00 | 0.00 | 3A7 |
| ATOM | 296 | C | GLN | 78 | 25.375 | −18.037 | −8.023 | 1.00 | 0.00 | 3A7 |
| ATOM | 297 | O | GLN | 78 | 25.715 | −18.949 | −8.771 | 1.00 | 0.00 | 3A7 |
| ATOM | 298 | N | GLN | 79 | 24.653 | −18.277 | −6.903 | 1.00 | 0.00 | 3A7 |
| ATOM | 299 | CA | GLN | 79 | 24.270 | −19.620 | −6.516 | 1.00 | 0.00 | 3A7 |
| ATOM | 300 | CB | GLN | 79 | 24.718 | −19.978 | −5.085 | 1.00 | 0.00 | 3A7 |
| ATOM | 301 | CG | GLN | 79 | 26.242 | −19.895 | −4.897 | 1.00 | 0.00 | 3A7 |
| ATOM | 302 | CD | GLN | 79 | 26.582 | −20.276 | −3.453 | 1.00 | 0.00 | 3A7 |
| ATOM | 303 | OE1 | GLN | 79 | 26.176 | −19.589 | −2.508 | 1.00 | 0.00 | 3A7 |
| ATOM | 304 | NE2 | GLN | 79 | 27.346 | −21.399 | −3.294 | 1.00 | 0.00 | 3A7 |
| ATOM | 305 | C | GLN | 79 | 22.765 | −19.774 | −6.615 | 1.00 | 0.00 | 3A7 |
| ATOM | 306 | O | GLN | 79 | 22.037 | −18.847 | −6.260 | 1.00 | 0.00 | 3A7 |
| ATOM | 307 | N | PRO | 80 | 22.241 | −20.926 | −7.069 | 1.00 | 0.00 | 3A7 |
| ATOM | 308 | CA | PRO | 80 | 20.824 | −21.225 | −7.042 | 1.00 | 0.00 | 3A7 |
| ATOM | 309 | CD | PRO | 80 | 22.976 | −21.867 | −7.907 | 1.00 | 0.00 | 3A7 |
| ATOM | 310 | CB | PRO | 80 | 20.605 | −22.191 | −8.214 | 1.00 | 0.00 | 3A7 |
| ATOM | 311 | CG | PRO | 80 | 21.950 | −22.915 | −8.360 | 1.00 | 0.00 | 3A7 |
| ATOM | 312 | C | PRO | 80 | 20.502 | −21.899 | −5.737 | 1.00 | 0.00 | 3A7 |
| ATOM | 313 | O | PRO | 80 | 21.269 | −22.746 | −5.277 | 1.00 | 0.00 | 3A7 |
| ATOM | 314 | N | MET | 81 | 19.342 | −21.571 | −5.140 | 1.00 | 0.00 | 3A7 |
| ATOM | 315 | CA | MET | 81 | 18.883 | −22.233 | −3.952 | 1.00 | 0.00 | 3A7 |
| ATOM | 316 | CB | MET | 81 | 19.098 | −21.392 | −2.669 | 1.00 | 0.00 | 3A7 |
| ATOM | 317 | CG | MET | 81 | 20.582 | −21.043 | −2.428 | 1.00 | 0.00 | 3A7 |
| ATOM | 318 | SD | MET | 81 | 20.947 | −20.209 | −0.854 | 1.00 | 0.00 | 3A7 |
| ATOM | 319 | CE | MET | 81 | 20.120 | −18.650 | −1.276 | 1.00 | 0.00 | 3A7 |
| ATOM | 320 | C | MET | 81 | 17.447 | −22.487 | −4.177 | 1.00 | 0.00 | 3A7 |
| ATOM | 321 | O | MET | 81 | 16.677 | −21.572 | −4.447 | 1.00 | 0.00 | 3A7 |
| ATOM | 322 | N | LEU | 82 | 17.035 | −23.763 | −4.052 | 1.00 | 0.00 | 3A7 |
| ATOM | 323 | CA | LEU | 82 | 15.657 | −24.130 | −4.201 | 1.00 | 0.00 | 3A7 |
| ATOM | 324 | CB | LEU | 82 | 15.468 | −25.434 | −4.990 | 1.00 | 0.00 | 3A7 |
| ATOM | 325 | CG | LEU | 82 | 15.574 | −25.122 | −6.509 | 1.00 | 0.00 | 3A7 |
| ATOM | 326 | CD1 | LEU | 82 | 16.903 | −25.569 | −7.137 | 1.00 | 0.00 | 3A7 |
| ATOM | 327 | CD2 | LEU | 82 | 14.352 | −25.622 | −7.300 | 1.00 | 0.00 | 3A7 |
| ATOM | 328 | C | LEU | 82 | 15.022 | −24.179 | −2.843 | 1.00 | 0.00 | 3A7 |
| ATOM | 329 | O | LEU | 82 | 15.439 | −24.903 | −1.947 | 1.00 | 0.00 | 3A7 |
| ATOM | 330 | N | ALA | 83 | 13.975 | −23.351 | −2.673 | 1.00 | 0.00 | 3A7 |
| ATOM | 331 | CA | ALA | 83 | 13.243 | −23.210 | −1.443 | 1.00 | 0.00 | 3A7 |
| ATOM | 332 | CB | ALA | 83 | 12.731 | −21.792 | −1.267 | 1.00 | 0.00 | 3A7 |
| ATOM | 333 | C | ALA | 83 | 12.040 | −24.096 | −1.430 | 1.00 | 0.00 | 3A7 |
| ATOM | 334 | O | ALA | 83 | 11.193 | −24.025 | −2.320 | 1.00 | 0.00 | 3A7 |
| ATOM | 335 | N | ILE | 84 | 11.963 | −24.967 | −0.399 | 1.00 | 0.00 | 3A7 |
| ATOM | 336 | CA | ILE | 84 | 10.949 | −25.985 | −0.290 | 1.00 | 0.00 | 3A7 |
| ATOM | 337 | CB | ILE | 84 | 11.517 | −27.364 | 0.009 | 1.00 | 0.00 | 3A7 |
| ATOM | 338 | CG2 | ILE | 84 | 10.376 | −28.307 | 0.374 | 1.00 | 0.00 | 3A7 |
| ATOM | 339 | CG1 | ILE | 84 | 12.173 | −28.016 | −1.243 | 1.00 | 0.00 | 3A7 |
| ATOM | 340 | CD | ILE | 84 | 13.207 | −27.201 | −2.013 | 1.00 | 0.00 | 3A7 |
| ATOM | 341 | C | ILE | 84 | 10.004 | −25.576 | 0.802 | 1.00 | 0.00 | 3A7 |
| ATOM | 342 | O | ILE | 84 | 10.404 | −25.289 | 1.921 | 1.00 | 0.00 | 3A7 |
| ATOM | 343 | N | THR | 85 | 8.689 | −25.597 | 0.509 | 1.00 | 0.00 | 3A7 |
| ATOM | 344 | CA | THR | 85 | 7.672 | −25.139 | 1.413 | 1.00 | 0.00 | 3A7 |
| ATOM | 345 | CB | THR | 85 | 7.073 | −23.819 | 0.999 | 1.00 | 0.00 | 3A7 |
| ATOM | 346 | OG1 | THR | 85 | 7.093 | −23.627 | −0.412 | 1.00 | 0.00 | 3A7 |
| ATOM | 347 | CG2 | THR | 85 | 7.922 | −22.782 | 1.725 | 1.00 | 0.00 | 3A7 |
| ATOM | 348 | C | THR | 85 | 6.562 | −26.125 | 1.486 | 1.00 | 0.00 | 3A7 |
| ATOM | 349 | O | THR | 85 | 6.050 | −26.730 | 0.619 | 1.00 | 0.00 | 3A7 |
| ATOM | 350 | N | ASP | 86 | 5.995 | −26.396 | 2.502 | 1.00 | 0.00 | 3A7 |
| ATOM | 351 | CA | ASP | 86 | 4.810 | −27.263 | 2.510 | 1.00 | 0.00 | 3A7 |
| ATOM | 352 | CB | ASP | 86 | 3.722 | −27.421 | 1.361 | 1.00 | 0.00 | 3A7 |
| ATOM | 353 | CG | ASP | 86 | 3.168 | −26.055 | 0.952 | 1.00 | 0.00 | 3A7 |
| ATOM | 354 | OD1 | ASP | 86 | 2.591 | −25.362 | 1.833 | 1.00 | 0.00 | 3A7 |
| ATOM | 355 | OD2 | ASP | 86 | 3.312 | −25.692 | −0.246 | 1.00 | 0.00 | 3A7 |
| ATOM | 356 | C | ASP | 86 | 5.268 | −28.602 | 3.011 | 1.00 | 0.00 | 3A7 |
| ATOM | 357 | O | ASP | 86 | 6.399 | −28.994 | 2.738 | 1.00 | 0.00 | 3A7 |
| ATOM | 358 | N | PRO | 87 | 4.433 | −29.324 | 3.756 | 1.00 | 0.00 | 3A7 |
| ATOM | 359 | CA | PRO | 87 | 4.827 | −30.519 | 4.472 | 1.00 | 0.00 | 3A7 |
| ATOM | 360 | CD | PRO | 87 | 3.262 | −28.734 | 4.388 | 1.00 | 0.00 | 3A7 |
| ATOM | 361 | CB | PRO | 87 | 3.599 | −30.942 | 5.276 | 1.00 | 0.00 | 3A7 |
| ATOM | 362 | CG | PRO | 87 | 2.505 | −29.914 | 4.977 | 1.00 | 0.00 | 3A7 |
| ATOM | 363 | C | PRO | 87 | 5.202 | −31.641 | 3.571 | 1.00 | 0.00 | 3A7 |
| ATOM | 364 | O | PRO | 87 | 6.112 | −32.375 | 3.920 | 1.00 | 0.00 | 3A7 |
| ATOM | 365 | N | ASP | 88 | 4.533 | −31.800 | 2.413 | 1.00 | 0.00 | 3A7 |
| ATOM | 366 | CA | ASP | 88 | 4.791 | −32.846 | 1.455 | 1.00 | 0.00 | 3A7 |
| ATOM | 367 | CB | ASP | 88 | 3.829 | −32.701 | 0.270 | 1.00 | 0.00 | 3A7 |
| ATOM | 368 | CG | ASP | 88 | 2.381 | −32.826 | 0.749 | 1.00 | 0.00 | 3A7 |
| ATOM | 369 | OD1 | ASP | 88 | 2.122 | −33.640 | 1.676 | 1.00 | 0.00 | 3A7 |
| ATOM | 370 | OD2 | ASP | 88 | 1.516 | −32.094 | 0.198 | 1.00 | 0.00 | 3A7 |
| ATOM | 371 | C | ASP | 88 | 6.196 | −32.787 | 0.909 | 1.00 | 0.00 | 3A7 |
| ATOM | 372 | O | ASP | 88 | 6.874 | −33.798 | 0.830 | 1.00 | 0.00 | 3A7 |
| ATOM | 373 | N | MET | 89 | 6.691 | −31.588 | 0.554 | 1.00 | 0.00 | 3A7 |
| ATOM | 374 | CA | MET | 89 | 8.014 | −31.388 | 0.009 | 1.00 | 0.00 | 3A7 |
| ATOM | 375 | CB | MET | 89 | 8.081 | −30.073 | −0.773 | 1.00 | 0.00 | 3A7 |
| ATOM | 376 | CG | MET | 89 | 7.065 | −30.035 | −1.918 | 1.00 | 0.00 | 3A7 |
| ATOM | 377 | SD | MET | 89 | 5.914 | −28.663 | −1.731 | 1.00 | 0.00 | 3A7 |
| ATOM | 378 | CE | MET | 89 | 7.247 | −27.425 | −1.755 | 1.00 | 0.00 | 3A7 |
| ATOM | 379 | C | MET | 89 | 9.069 | −31.390 | 1.081 | 1.00 | 0.00 | 3A7 |
| ATOM | 380 | O | MET | 89 | 10.152 | −31.936 | 0.893 | 1.00 | 0.00 | 3A7 |
| ATOM | 381 | N | ILE | 90 | 8.754 | −30.824 | 2.267 | 1.00 | 0.00 | 3A7 |
| ATOM | 382 | CA | ILE | 90 | 9.657 | −30.812 | 3.398 | 1.00 | 0.00 | 3A7 |
| ATOM | 383 | CB | ILE | 90 | 9.126 | −29.908 | 4.501 | 1.00 | 0.00 | 3A7 |
| ATOM | 384 | CG2 | ILE | 90 | 9.952 | −30.036 | 5.807 | 1.00 | 0.00 | 3A7 |
| ATOM | 385 | CG1 | ILE | 90 | 9.060 | −28.467 | 3.973 | 1.00 | 0.00 | 3A7 |
| ATOM | 386 | CD | ILE | 90 | 10.436 | −27.853 | 3.815 | 1.00 | 0.00 | 3A7 |
| ATOM | 387 | C | ILE | 90 | 9.898 | −32.218 | 3.920 | 1.00 | 0.00 | 3A7 |
| ATOM | 388 | O | ILE | 90 | 11.016 | −32.588 | 4.248 | 1.00 | 0.00 | 3A7 |
| ATOM | 389 | N | LYS | 91 | 8.877 | −33.100 | 3.925 | 1.00 | 0.00 | 3A7 |
| ATOM | 390 | CA | LYS | 91 | 9.058 | −34.479 | 4.328 | 1.00 | 0.00 | 3A7 |
| ATOM | 391 | CB | LYS | 91 | 7.722 | −35.240 | 4.361 | 1.00 | 0.00 | 3A7 |
| ATOM | 392 | CG | LYS | 91 | 7.790 | −36.619 | 5.036 | 1.00 | 0.00 | 3A7 |
| ATOM | 393 | CD | LYS | 91 | 6.407 | −37.269 | 5.192 | 1.00 | 0.00 | 3A7 |
| ATOM | 394 | CE | LYS | 91 | 6.465 | −38.628 | 5.898 | 1.00 | 0.00 | 3A7 |
| ATOM | 395 | NZ | LYS | 91 | 5.108 | −39.206 | 6.031 | 1.00 | 0.00 | 3A7 |
| ATOM | 396 | C | LYS | 91 | 10.010 | −35.209 | 3.414 | 1.00 | 0.00 | 3A7 |
| ATOM | 397 | O | LYS | 91 | 10.824 | −36.021 | 3.838 | 1.00 | 0.00 | 3A7 |
| ATOM | 398 | N | THR | 92 | 9.969 | −34.861 | 2.113 | 1.00 | 0.00 | 3A7 |
| ATOM | 399 | CA | THR | 92 | 10.864 | −35.382 | 1.113 | 1.00 | 0.00 | 3A7 |
| ATOM | 400 | CB | THR | 92 | 10.441 | −34.980 | −0.272 | 1.00 | 0.00 | 3A7 |
| ATOM | 401 | OG1 | THR | 92 | 9.047 | −35.206 | −0.430 | 1.00 | 0.00 | 3A7 |
| ATOM | 402 | CG2 | THR | 92 | 11.180 | −35.836 | −1.323 | 1.00 | 0.00 | 3A7 |
| ATOM | 403 | C | THR | 92 | 12.263 | −34.904 | 1.326 | 1.00 | 0.00 | 3A7 |
| ATOM | 404 | O | THR | 92 | 13.222 | −35.657 | 1.196 | 1.00 | 0.00 | 3A7 |
| ATOM | 405 | N | VAL | 93 | 12.395 | −33.620 | 1.730 | 1.00 | 0.00 | 3A7 |
| ATOM | 406 | CA | VAL | 93 | 13.674 | −33.006 | 1.938 | 1.00 | 0.00 | 3A7 |
| ATOM | 407 | CB | VAL | 93 | 13.628 | −31.500 | 2.127 | 1.00 | 0.00 | 3A7 |
| ATOM | 408 | CG1 | VAL | 93 | 12.833 | −30.752 | 1.061 | 1.00 | 0.00 | 3A7 |
| ATOM | 409 | CG2 | VAL | 93 | 13.354 | −31.074 | 3.585 | 1.00 | 0.00 | 3A7 |
| ATOM | 410 | C | VAL | 93 | 14.483 | −33.705 | 3.034 | 1.00 | 0.00 | 3A7 |
| ATOM | 411 | O | VAL | 93 | 15.703 | −33.627 | 3.002 | 1.00 | 0.00 | 3A7 |
| ATOM | 412 | N | LEU | 94 | 13.861 | −34.522 | 3.946 | 1.00 | 0.00 | 3A7 |
| ATOM | 413 | CA | LEU | 94 | 14.509 | −35.450 | 4.889 | 1.00 | 0.00 | 3A7 |
| ATOM | 414 | CB | LEU | 94 | 13.538 | −36.540 | 5.429 | 1.00 | 0.00 | 3A7 |
| ATOM | 415 | CG | LEU | 94 | 12.454 | −36.028 | 6.397 | 1.00 | 0.00 | 3A7 |
| ATOM | 416 | CD1 | LEU | 94 | 11.435 | −37.136 | 6.726 | 1.00 | 0.00 | 3A7 |
| ATOM | 417 | CD2 | LEU | 94 | 13.079 | −35.489 | 7.687 | 1.00 | 0.00 | 3A7 |
| ATOM | 418 | C | LEU | 94 | 15.673 | −36.253 | 4.325 | 1.00 | 0.00 | 3A7 |
| ATOM | 419 | O | LEU | 94 | 16.786 | −36.171 | 4.842 | 1.00 | 0.00 | 3A7 |
| ATOM | 420 | N | VAL | 95 | 15.389 | −37.041 | 3.249 | 1.00 | 0.00 | 3A7 |
| ATOM | 421 | CA | VAL | 95 | 16.254 | −37.904 | 2.446 | 1.00 | 0.00 | 3A7 |
| ATOM | 422 | CB | VAL | 95 | 17.146 | −37.147 | 1.449 | 1.00 | 0.00 | 3A7 |
| ATOM | 423 | CG1 | VAL | 95 | 18.293 | −36.331 | 2.088 | 1.00 | 0.00 | 3A7 |
| ATOM | 424 | CG2 | VAL | 95 | 17.667 | −38.131 | 0.379 | 1.00 | 0.00 | 3A7 |
| ATOM | 425 | C | VAL | 95 | 17.019 | −38.932 | 3.269 | 1.00 | 0.00 | 3A7 |
| ATOM | 426 | O | VAL | 95 | 17.910 | −38.606 | 4.052 | 1.00 | 0.00 | 3A7 |
| ATOM | 427 | N | LYS | 96 | 16.651 | −40.226 | 3.099 | 1.00 | 0.00 | 3A7 |
| ATOM | 428 | CA | LYS | 96 | 17.216 | −41.310 | 3.864 | 1.00 | 0.00 | 3A7 |
| ATOM | 429 | CB | LYS | 96 | 16.284 | −41.757 | 5.021 | 1.00 | 0.00 | 3A7 |
| ATOM | 430 | CG | LYS | 96 | 16.863 | −42.863 | 5.922 | 1.00 | 0.00 | 3A7 |
| ATOM | 431 | CD | LYS | 96 | 15.917 | −43.296 | 7.055 | 1.00 | 0.00 | 3A7 |
| ATOM | 432 | CE | LYS | 96 | 15.646 | −42.211 | 8.105 | 1.00 | 0.00 | 3A7 |
| ATOM | 433 | NZ | LYS | 96 | 16.899 | −41.808 | 8.783 | 1.00 | 0.00 | 3A7 |
| ATOM | 434 | C | LYS | 96 | 17.438 | −42.463 | 2.917 | 1.00 | 0.00 | 3A7 |
| ATOM | 435 | O | LYS | 96 | 16.460 | −42.998 | 2.397 | 1.00 | 0.00 | 3A7 |
| ATOM | 436 | N | GLU | 97 | 18.697 | −42.930 | 2.649 | 1.00 | 0.00 | 3A7 |
| ATOM | 437 | CA | GLU | 97 | 20.025 | −42.451 | 3.016 | 1.00 | 0.00 | 3A7 |
| ATOM | 438 | CB | GLU | 97 | 20.351 | −41.024 | 2.498 | 1.00 | 0.00 | 3A7 |
| ATOM | 439 | CG | GLU | 97 | 20.271 | −40.894 | 0.966 | 1.00 | 0.00 | 3A7 |
| ATOM | 440 | CD | GLU | 97 | 21.342 | −41.763 | 0.314 | 1.00 | 0.00 | 3A7 |
| ATOM | 441 | OE1 | GLU | 97 | 20.968 | −42.695 | −0.447 | 1.00 | 0.00 | 3A7 |
| ATOM | 442 | OE2 | GLU | 97 | 22.549 | −41.504 | 0.567 | 1.00 | 0.00 | 3A7 |
| ATOM | 443 | C | GLU | 97 | 20.318 | −42.531 | 4.499 | 1.00 | 0.00 | 3A7 |
| ATOM | 444 | O | GLU | 97 | 20.158 | −41.560 | 5.238 | 1.00 | 0.00 | 3A7 |
| ATOM | 445 | N | CYS | 98 | 20.813 | −43.711 | 4.956 | 1.00 | 0.00 | 3A7 |
| ATOM | 446 | CA | CYS | 98 | 21.223 | −43.956 | 6.327 | 1.00 | 0.00 | 3A7 |
| ATOM | 447 | CB | CYS | 98 | 21.159 | −45.456 | 6.703 | 1.00 | 0.00 | 3A7 |
| ATOM | 448 | SG | CYS | 98 | 19.470 | −46.111 | 6.530 | 1.00 | 0.00 | 3A7 |
| ATOM | 449 | C | CYS | 98 | 22.633 | −43.454 | 6.538 | 1.00 | 0.00 | 3A7 |
| ATOM | 450 | O | CYS | 98 | 23.032 | −43.131 | 7.656 | 1.00 | 0.00 | 3A7 |
| ATOM | 451 | N | TYR | 99 | 23.399 | −43.358 | 5.423 | 1.00 | 0.00 | 3A7 |
| ATOM | 452 | CA | TYR | 99 | 24.680 | −42.699 | 5.352 | 1.00 | 0.00 | 3A7 |
| ATOM | 453 | CB | TYR | 99 | 25.794 | −43.602 | 4.775 | 1.00 | 0.00 | 3A7 |
| ATOM | 454 | CG | TYR | 99 | 26.067 | −44.708 | 5.749 | 1.00 | 0.00 | 3A7 |
| ATOM | 455 | CD1 | TYR | 99 | 25.528 | −45.981 | 5.553 | 1.00 | 0.00 | 3A7 |
| ATOM | 456 | CD2 | TYR | 99 | 26.855 | −44.471 | 6.875 | 1.00 | 0.00 | 3A7 |
| ATOM | 457 | CE1 | TYR | 99 | 25.771 | −47.000 | 6.470 | 1.00 | 0.00 | 3A7 |
| ATOM | 458 | CE2 | TYR | 99 | 27.103 | −45.488 | 7.791 | 1.00 | 0.00 | 3A7 |
| ATOM | 459 | CZ | TYR | 99 | 26.560 | −46.754 | 7.591 | 1.00 | 0.00 | 3A7 |
| ATOM | 460 | OH | TYR | 99 | 26.807 | −47.785 | 8.524 | 1.00 | 0.00 | 3A7 |
| ATOM | 461 | C | TYR | 99 | 24.459 | −41.520 | 4.445 | 1.00 | 0.00 | 3A7 |
| ATOM | 462 | O | TYR | 99 | 24.717 | −41.584 | 3.244 | 1.00 | 0.00 | 3A7 |
| ATOM | 463 | N | SER | 100 | 23.943 | −40.407 | 5.029 | 1.00 | 0.00 | 3A7 |
| ATOM | 464 | CA | SER | 100 | 23.548 | −39.211 | 4.317 | 1.00 | 0.00 | 3A7 |
| ATOM | 465 | CB | SER | 100 | 22.340 | −38.501 | 4.990 | 1.00 | 0.00 | 3A7 |
| ATOM | 466 | OG | SER | 100 | 22.547 | −38.275 | 6.381 | 1.00 | 0.00 | 3A7 |
| ATOM | 467 | C | SER | 100 | 24.721 | −38.267 | 4.207 | 1.00 | 0.00 | 3A7 |
| ATOM | 468 | O | SER | 100 | 25.295 | −37.848 | 5.211 | 1.00 | 0.00 | 3A7 |
| ATOM | 469 | N | VAL | 101 | 25.093 | −37.923 | 2.947 | 1.00 | 0.00 | 3A7 |
| ATOM | 470 | CA | VAL | 101 | 26.233 | −37.096 | 2.616 | 1.00 | 0.00 | 3A7 |
| ATOM | 471 | CB | VAL | 101 | 26.925 | −37.529 | 1.327 | 1.00 | 0.00 | 3A7 |
| ATOM | 472 | CG1 | VAL | 101 | 28.244 | −36.746 | 1.143 | 1.00 | 0.00 | 3A7 |
| ATOM | 473 | CG2 | VAL | 101 | 27.188 | −39.049 | 1.394 | 1.00 | 0.00 | 3A7 |
| ATOM | 474 | C | VAL | 101 | 25.777 | −35.663 | 2.512 | 1.00 | 0.00 | 3A7 |
| ATOM | 475 | O | VAL | 101 | 25.384 | −35.187 | 1.447 | 1.00 | 0.00 | 3A7 |
| ATOM | 476 | N | PHE | 102 | 25.851 | −34.944 | 3.658 | 1.00 | 0.00 | 3A7 |
| ATOM | 477 | CA | PHE | 102 | 25.588 | −33.531 | 3.774 | 1.00 | 0.00 | 3A7 |
| ATOM | 478 | CB | PHE | 102 | 24.757 | −33.208 | 5.051 | 1.00 | 0.00 | 3A7 |
| ATOM | 479 | CG | PHE | 102 | 24.515 | −31.734 | 5.284 | 1.00 | 0.00 | 3A7 |
| ATOM | 480 | CD1 | PHE | 102 | 24.224 | −30.854 | 4.236 | 1.00 | 0.00 | 3A7 |
| ATOM | 481 | CD2 | PHE | 102 | 24.602 | −31.227 | 6.582 | 1.00 | 0.00 | 3A7 |
| ATOM | 482 | CE1 | PHE | 102 | 24.063 | −29.493 | 4.481 | 1.00 | 0.00 | 3A7 |
| ATOM | 483 | CE2 | PHE | 102 | 24.424 | −29.870 | 6.830 | 1.00 | 0.00 | 3A7 |
| ATOM | 484 | CZ | PHE | 102 | 24.163 | −29.000 | 5.777 | 1.00 | 0.00 | 3A7 |
| ATOM | 485 | C | PHE | 102 | 26.945 | −32.887 | 3.838 | 1.00 | 0.00 | 3A7 |
| ATOM | 486 | O | PHE | 102 | 27.728 | −33.174 | 4.741 | 1.00 | 0.00 | 3A7 |
| ATOM | 487 | N | THR | 103 | 27.238 | −31.980 | 2.869 | 1.00 | 0.00 | 3A7 |
| ATOM | 488 | CA | THR | 103 | 28.502 | −31.281 | 2.757 | 1.00 | 0.00 | 3A7 |
| ATOM | 489 | CB | THR | 103 | 28.814 | −30.904 | 1.308 | 1.00 | 0.00 | 3A7 |
| ATOM | 490 | OG1 | THR | 103 | 30.168 | −30.496 | 1.135 | 1.00 | 0.00 | 3A7 |
| ATOM | 491 | CG2 | THR | 103 | 27.858 | −29.816 | 0.779 | 1.00 | 0.00 | 3A7 |
| ATOM | 492 | C | THR | 103 | 28.484 | −30.083 | 3.685 | 1.00 | 0.00 | 3A7 |
| ATOM | 493 | O | THR | 103 | 27.430 | −29.672 | 4.169 | 1.00 | 0.00 | 3A7 |
| ATOM | 494 | N | ASN | 104 | 29.682 | −29.507 | 3.955 | 1.00 | 0.00 | 3A7 |
| ATOM | 495 | CA | ASN | 104 | 29.888 | −28.430 | 4.899 | 1.00 | 0.00 | 3A7 |
| ATOM | 496 | CB | ASN | 104 | 31.378 | −28.320 | 5.343 | 1.00 | 0.00 | 3A7 |
| ATOM | 497 | CG | ASN | 104 | 32.353 | −28.168 | 4.161 | 1.00 | 0.00 | 3A7 |
| ATOM | 498 | OD1 | ASN | 104 | 32.706 | −27.047 | 3.775 | 1.00 | 0.00 | 3A7 |
| ATOM | 499 | ND2 | ASN | 104 | 32.796 | −29.333 | 3.597 | 1.00 | 0.00 | 3A7 |
| ATOM | 500 | C | ASN | 104 | 29.418 | −27.105 | 4.342 | 1.00 | 0.00 | 3A7 |
| ATOM | 501 | O | ASN | 104 | 29.471 | −26.868 | 3.135 | 1.00 | 0.00 | 3A7 |
| ATOM | 502 | N | ARG | 105 | 28.961 | −26.205 | 5.250 | 1.00 | 0.00 | 3A7 |
| ATOM | 503 | CA | ARG | 105 | 28.538 | −24.859 | 4.929 | 1.00 | 0.00 | 3A7 |
| ATOM | 504 | CB | ARG | 105 | 27.556 | −24.275 | 5.967 | 1.00 | 0.00 | 3A7 |
| ATOM | 505 | CG | ARG | 105 | 26.214 | −25.018 | 6.037 | 1.00 | 0.00 | 3A7 |
| ATOM | 506 | CD | ARG | 105 | 25.300 | −24.418 | 7.111 | 1.00 | 0.00 | 3A7 |
| ATOM | 507 | NE | ARG | 105 | 24.029 | −25.205 | 7.173 | 1.00 | 0.00 | 3A7 |
| ATOM | 508 | CZ | ARG | 105 | 23.007 | −24.845 | 8.007 | 1.00 | 0.00 | 3A7 |
| ATOM | 509 | NH1 | ARG | 105 | 21.860 | −25.583 | 8.027 | 1.00 | 0.00 | 3A7 |
| ATOM | 510 | NH2 | ARG | 105 | 23.124 | −23.752 | 8.816 | 1.00 | 0.00 | 3A7 |
| ATOM | 511 | C | ARG | 105 | 29.756 | −23.977 | 4.884 | 1.00 | 0.00 | 3A7 |
| ATOM | 512 | O | ARG | 105 | 30.703 | −24.170 | 5.645 | 1.00 | 0.00 | 3A7 |
| ATOM | 513 | N | ARG | 106 | 29.750 | −22.991 | 3.955 | 1.00 | 0.00 | 3A7 |
| ATOM | 514 | CA | ARG | 106 | 30.909 | −22.177 | 3.639 | 1.00 | 0.00 | 3A7 |
| ATOM | 515 | CB | ARG | 106 | 30.852 | −21.586 | 2.205 | 1.00 | 0.00 | 3A7 |
| ATOM | 516 | CG | ARG | 106 | 30.675 | −22.664 | 1.121 | 1.00 | 0.00 | 3A7 |
| ATOM | 517 | CD | ARG | 106 | 30.504 | −22.088 | −0.292 | 1.00 | 0.00 | 3A7 |
| ATOM | 518 | NE | ARG | 106 | 31.739 | −21.318 | −0.652 | 1.00 | 0.00 | 3A7 |
| ATOM | 519 | CZ | ARG | 106 | 31.841 | −20.619 | −1.824 | 1.00 | 0.00 | 3A7 |
| ATOM | 520 | NH1 | ARG | 106 | 32.985 | −19.927 | −2.100 | 1.00 | 0.00 | 3A7 |
| ATOM | 521 | NH2 | ARG | 106 | 30.809 | −20.607 | −2.718 | 1.00 | 0.00 | 3A7 |
| ATOM | 522 | C | ARG | 106 | 31.190 | −21.087 | 4.647 | 1.00 | 0.00 | 3A7 |
| ATOM | 523 | O | ARG | 106 | 32.357 | −20.971 | 5.015 | 1.00 | 0.00 | 3A7 |
| ATOM | 524 | N | PRO | 107 | 30.250 | −20.287 | 5.178 | 1.00 | 0.00 | 3A7 |
| ATOM | 525 | CA | PRO | 107 | 30.565 | −19.212 | 6.105 | 1.00 | 0.00 | 3A7 |
| ATOM | 526 | CD | PRO | 107 | 28.890 | −20.159 | 4.663 | 1.00 | 0.00 | 3A7 |
| ATOM | 527 | CB | PRO | 107 | 29.245 | −18.456 | 6.287 | 1.00 | 0.00 | 3A7 |
| ATOM | 528 | CG | PRO | 107 | 28.480 | −18.724 | 4.991 | 1.00 | 0.00 | 3A7 |
| ATOM | 529 | C | PRO | 107 | 31.061 | −19.732 | 7.435 | 1.00 | 0.00 | 3A7 |
| ATOM | 530 | O | PRO | 107 | 31.834 | −19.045 | 8.098 | 1.00 | 0.00 | 3A7 |
| ATOM | 531 | N | PHE | 108 | 30.645 | −20.949 | 7.846 | 1.00 | 0.00 | 3A7 |
| ATOM | 532 | CA | PHE | 108 | 30.989 | −21.534 | 9.116 | 1.00 | 0.00 | 3A7 |
| ATOM | 533 | CB | PHE | 108 | 29.984 | −22.623 | 9.543 | 1.00 | 0.00 | 3A7 |
| ATOM | 534 | CG | PHE | 108 | 28.575 | −22.099 | 9.639 | 1.00 | 0.00 | 3A7 |
| ATOM | 535 | CD1 | PHE | 108 | 27.823 | −21.852 | 8.488 | 1.00 | 0.00 | 3A7 |
| ATOM | 536 | CD2 | PHE | 108 | 27.969 | −21.924 | 10.885 | 1.00 | 0.00 | 3A7 |
| ATOM | 537 | CE1 | PHE | 108 | 26.496 | −21.446 | 8.575 | 1.00 | 0.00 | 3A7 |
| ATOM | 538 | CE2 | PHE | 108 | 26.638 | −21.528 | 10.976 | 1.00 | 0.00 | 3A7 |
| ATOM | 539 | CZ | PHE | 108 | 25.898 | −21.298 | 9.821 | 1.00 | 0.00 | 3A7 |
| ATOM | 540 | C | PHE | 108 | 32.360 | −22.153 | 9.070 | 1.00 | 0.00 | 3A7 |
| ATOM | 541 | O | PHE | 108 | 33.016 | −22.269 | 10.095 | 1.00 | 0.00 | 3A7 |
| ATOM | 542 | N | GLY | 109 | 32.846 | −22.555 | 7.875 | 1.00 | 0.00 | 3A7 |
| ATOM | 543 | CA | GLY | 109 | 34.159 | −23.135 | 7.691 | 1.00 | 0.00 | 3A7 |
| ATOM | 544 | C | GLY | 109 | 35.333 | −22.254 | 8.056 | 1.00 | 0.00 | 3A7 |
| ATOM | 545 | O | GLY | 109 | 36.321 | −22.774 | 8.561 | 1.00 | 0.00 | 3A7 |
| ATOM | 546 | N | PRO | 110 | 35.267 | −20.938 | 7.853 | 1.00 | 0.00 | 3A7 |
| ATOM | 547 | CA | PRO | 110 | 36.311 | −20.023 | 8.270 | 1.00 | 0.00 | 3A7 |
| ATOM | 548 | CD | PRO | 110 | 34.680 | −20.430 | 6.620 | 1.00 | 0.00 | 3A7 |
| ATOM | 549 | CB | PRO | 110 | 35.805 | −18.682 | 7.743 | 1.00 | 0.00 | 3A7 |
| ATOM | 550 | CG | PRO | 110 | 35.292 | −19.054 | 6.361 | 1.00 | 0.00 | 3A7 |
| ATOM | 551 | C | PRO | 110 | 36.590 | −19.895 | 9.765 | 1.00 | 0.00 | 3A7 |
| ATOM | 552 | O | PRO | 110 | 37.717 | −19.525 | 10.094 | 1.00 | 0.00 | 3A7 |
| ATOM | 553 | N | VAL | 111 | 35.620 | −20.153 | 10.690 | 1.00 | 0.00 | 3A7 |
| ATOM | 554 | CA | VAL | 111 | 35.799 | −19.847 | 12.107 | 1.00 | 0.00 | 3A7 |
| ATOM | 555 | CB | VAL | 111 | 34.495 | −19.678 | 12.890 | 1.00 | 0.00 | 3A7 |
| ATOM | 556 | CG1 | VAL | 111 | 33.623 | −18.645 | 12.147 | 1.00 | 0.00 | 3A7 |
| ATOM | 557 | CG2 | VAL | 111 | 33.745 | −21.007 | 13.118 | 1.00 | 0.00 | 3A7 |
| ATOM | 558 | C | VAL | 111 | 36.707 | −20.855 | 12.787 | 1.00 | 0.00 | 3A7 |
| ATOM | 559 | O | VAL | 111 | 36.539 | −22.067 | 12.658 | 1.00 | 0.00 | 3A7 |
| ATOM | 560 | N | GLY | 112 | 37.725 | −20.325 | 13.502 | 1.00 | 0.00 | 3A7 |
| ATOM | 561 | CA | GLY | 112 | 38.761 | −21.102 | 14.130 | 1.00 | 0.00 | 3A7 |
| ATOM | 562 | C | GLY | 112 | 40.044 | −20.384 | 13.863 | 1.00 | 0.00 | 3A7 |
| ATOM | 563 | O | GLY | 112 | 40.101 | −19.155 | 13.891 | 1.00 | 0.00 | 3A7 |
| ATOM | 564 | N | PHE | 113 | 41.113 | −21.162 | 13.582 | 1.00 | 0.00 | 3A7 |
| ATOM | 565 | CA | PHE | 113 | 42.395 | −20.640 | 13.182 | 1.00 | 0.00 | 3A7 |
| ATOM | 566 | CB | PHE | 113 | 43.376 | −20.476 | 14.380 | 1.00 | 0.00 | 3A7 |
| ATOM | 567 | CG | PHE | 113 | 44.692 | −19.854 | 13.980 | 1.00 | 0.00 | 3A7 |
| ATOM | 568 | CD1 | PHE | 113 | 45.891 | −20.468 | 14.346 | 1.00 | 0.00 | 3A7 |
| ATOM | 569 | CD2 | PHE | 113 | 44.738 | −18.671 | 13.239 | 1.00 | 0.00 | 3A7 |
| ATOM | 570 | CE1 | PHE | 113 | 47.113 | −19.918 | 13.971 | 1.00 | 0.00 | 3A7 |
| ATOM | 571 | CE2 | PHE | 113 | 45.959 | −18.120 | 12.861 | 1.00 | 0.00 | 3A7 |
| ATOM | 572 | CZ | PHE | 113 | 47.148 | −18.744 | 13.226 | 1.00 | 0.00 | 3A7 |
| ATOM | 573 | C | PHE | 113 | 42.890 | −21.648 | 12.181 | 1.00 | 0.00 | 3A7 |
| ATOM | 574 | O | PHE | 113 | 43.853 | −22.372 | 12.431 | 1.00 | 0.00 | 3A7 |
| ATOM | 575 | N | MET | 114 | 42.193 | −21.708 | 11.011 | 1.00 | 0.00 | 3A7 |
| ATOM | 576 | CA | MET | 114 | 42.417 | −22.600 | 9.882 | 1.00 | 0.00 | 3A7 |
| ATOM | 577 | CB | MET | 114 | 43.873 | −22.624 | 9.331 | 1.00 | 0.00 | 3A7 |
| ATOM | 578 | CG | MET | 114 | 44.322 | −21.313 | 8.647 | 1.00 | 0.00 | 3A7 |
| ATOM | 579 | SD | MET | 114 | 44.745 | −19.936 | 9.761 | 1.00 | 0.00 | 3A7 |
| ATOM | 580 | CE | MET | 114 | 45.205 | −18.779 | 8.440 | 1.00 | 0.00 | 3A7 |
| ATOM | 581 | C | MET | 114 | 41.966 | −24.012 | 10.217 | 1.00 | 0.00 | 3A7 |
| ATOM | 582 | O | MET | 114 | 41.563 | −24.294 | 11.346 | 1.00 | 0.00 | 3A7 |
| ATOM | 583 | N | LYS | 115 | 42.028 | −24.924 | 9.208 | 1.00 | 0.00 | 3A7 |
| ATOM | 584 | CA | LYS | 115 | 41.701 | −26.343 | 9.268 | 1.00 | 0.00 | 3A7 |
| ATOM | 585 | CB | LYS | 115 | 42.359 | −27.158 | 10.423 | 1.00 | 0.00 | 3A7 |
| ATOM | 586 | CG | LYS | 115 | 43.868 | −27.432 | 10.251 | 1.00 | 0.00 | 3A7 |
| ATOM | 587 | CD | LYS | 115 | 44.814 | −26.252 | 10.533 | 1.00 | 0.00 | 3A7 |
| ATOM | 588 | CE | LYS | 115 | 44.796 | −25.758 | 11.988 | 1.00 | 0.00 | 3A7 |
| ATOM | 589 | NZ | LYS | 115 | 45.206 | −26.832 | 12.922 | 1.00 | 0.00 | 3A7 |
| ATOM | 590 | C | LYS | 115 | 40.202 | −26.569 | 9.300 | 1.00 | 0.00 | 3A7 |
| ATOM | 591 | O | LYS | 115 | 39.622 | −26.979 | 8.297 | 1.00 | 0.00 | 3A7 |
| ATOM | 592 | N | ASN | 116 | 39.579 | −26.341 | 10.491 | 1.00 | 0.00 | 3A7 |
| ATOM | 593 | CA | ASN | 116 | 38.192 | −26.560 | 10.889 | 1.00 | 0.00 | 3A7 |
| ATOM | 594 | CB | ASN | 116 | 37.101 | −25.724 | 10.133 | 1.00 | 0.00 | 3A7 |
| ATOM | 595 | CG | ASN | 116 | 36.831 | −26.079 | 8.657 | 1.00 | 0.00 | 3A7 |
| ATOM | 596 | OD1 | ASN | 116 | 37.384 | −25.453 | 7.745 | 1.00 | 0.00 | 3A7 |
| ATOM | 597 | ND2 | ASN | 116 | 35.919 | −27.074 | 8.437 | 1.00 | 0.00 | 3A7 |
| ATOM | 598 | C | ASN | 116 | 37.862 | −28.039 | 10.949 | 1.00 | 0.00 | 3A7 |
| ATOM | 599 | O | ASN | 116 | 38.029 | −28.777 | 9.979 | 1.00 | 0.00 | 3A7 |
| ATOM | 600 | N | ALA | 117 | 37.387 | −28.501 | 12.134 | 1.00 | 0.00 | 3A7 |
| ATOM | 601 | CA | ALA | 117 | 37.152 | −29.900 | 12.415 | 1.00 | 0.00 | 3A7 |
| ATOM | 602 | CB | ALA | 117 | 37.393 | −30.247 | 13.899 | 1.00 | 0.00 | 3A7 |
| ATOM | 603 | C | ALA | 117 | 35.750 | −30.294 | 12.024 | 1.00 | 0.00 | 3A7 |
| ATOM | 604 | O | ALA | 117 | 35.542 | −30.857 | 10.951 | 1.00 | 0.00 | 3A7 |
| ATOM | 605 | N | ILE | 118 | 34.758 | −30.027 | 12.909 | 1.00 | 0.00 | 3A7 |
| ATOM | 606 | CA | ILE | 118 | 33.403 | −30.498 | 12.732 | 1.00 | 0.00 | 3A7 |
| ATOM | 607 | CB | ILE | 118 | 33.060 | −31.666 | 13.661 | 1.00 | 0.00 | 3A7 |
| ATOM | 608 | CG2 | ILE | 118 | 33.620 | −32.948 | 13.008 | 1.00 | 0.00 | 3A7 |
| ATOM | 609 | CG1 | ILE | 118 | 33.625 | −31.523 | 15.097 | 1.00 | 0.00 | 3A7 |
| ATOM | 610 | CD | ILE | 118 | 32.902 | −30.511 | 15.983 | 1.00 | 0.00 | 3A7 |
| ATOM | 611 | C | ILE | 118 | 32.454 | −29.333 | 12.894 | 1.00 | 0.00 | 3A7 |
| ATOM | 612 | O | ILE | 118 | 31.481 | −29.399 | 13.643 | 1.00 | 0.00 | 3A7 |
| ATOM | 613 | N | SER | 119 | 32.700 | −28.231 | 12.139 | 1.00 | 0.00 | 3A7 |
| ATOM | 614 | CA | SER | 119 | 31.852 | −27.054 | 12.114 | 1.00 | 0.00 | 3A7 |
| ATOM | 615 | CB | SER | 119 | 32.666 | −25.747 | 11.972 | 1.00 | 0.00 | 3A7 |
| ATOM | 616 | OG | SER | 119 | 33.544 | −25.595 | 13.079 | 1.00 | 0.00 | 3A7 |
| ATOM | 617 | C | SER | 119 | 30.902 | −27.178 | 10.949 | 1.00 | 0.00 | 3A7 |
| ATOM | 618 | O | SER | 119 | 31.063 | −26.507 | 9.930 | 1.00 | 0.00 | 3A7 |
| ATOM | 619 | N | ILE | 120 | 29.898 | −28.096 | 11.093 | 1.00 | 0.00 | 3A7 |
| ATOM | 620 | CA | ILE | 120 | 28.963 | −28.576 | 10.086 | 1.00 | 0.00 | 3A7 |
| ATOM | 621 | CB | ILE | 120 | 28.220 | −27.497 | 9.294 | 1.00 | 0.00 | 3A7 |
| ATOM | 622 | CG2 | ILE | 120 | 27.196 | −28.166 | 8.349 | 1.00 | 0.00 | 3A7 |
| ATOM | 623 | CG1 | ILE | 120 | 27.521 | −26.482 | 10.237 | 1.00 | 0.00 | 3A7 |
| ATOM | 624 | CD | ILE | 120 | 26.467 | −27.096 | 11.166 | 1.00 | 0.00 | 3A7 |
| ATOM | 625 | C | ILE | 120 | 29.730 | −29.507 | 9.171 | 1.00 | 0.00 | 3A7 |
| ATOM | 626 | O | ILE | 120 | 30.456 | −29.081 | 8.276 | 1.00 | 0.00 | 3A7 |
| ATOM | 627 | N | ALA | 121 | 29.621 | −30.825 | 9.421 | 1.00 | 0.00 | 3A7 |
| ATOM | 628 | CA | ALA | 121 | 30.540 | −31.779 | 8.845 | 1.00 | 0.00 | 3A7 |
| ATOM | 629 | CB | ALA | 121 | 31.516 | −32.322 | 9.891 | 1.00 | 0.00 | 3A7 |
| ATOM | 630 | C | ALA | 121 | 29.781 | −32.895 | 8.190 | 1.00 | 0.00 | 3A7 |
| ATOM | 631 | O | ALA | 121 | 28.574 | −33.051 | 8.369 | 1.00 | 0.00 | 3A7 |
| ATOM | 632 | N | GLU | 122 | 30.519 | −33.699 | 7.384 | 1.00 | 0.00 | 3A7 |
| ATOM | 633 | CA | GLU | 122 | 30.006 | −34.785 | 6.580 | 1.00 | 0.00 | 3A7 |
| ATOM | 634 | CB | GLU | 122 | 30.832 | −34.988 | 5.292 | 1.00 | 0.00 | 3A7 |
| ATOM | 635 | CG | GLU | 122 | 31.075 | −33.676 | 4.532 | 1.00 | 0.00 | 3A7 |
| ATOM | 636 | CD | GLU | 122 | 31.734 | −33.979 | 3.190 | 1.00 | 0.00 | 3A7 |
| ATOM | 637 | OE1 | GLU | 122 | 31.094 | −34.679 | 2.360 | 1.00 | 0.00 | 3A7 |
| ATOM | 638 | OE2 | GLU | 122 | 32.884 | −33.510 | 2.975 | 1.00 | 0.00 | 3A7 |
| ATOM | 639 | C | GLU | 122 | 30.077 | −36.057 | 7.368 | 1.00 | 0.00 | 3A7 |
| ATOM | 640 | O | GLU | 122 | 30.753 | −36.091 | 8.393 | 1.00 | 0.00 | 3A7 |
| ATOM | 641 | N | ASP | 123 | 29.389 | −37.130 | 6.889 | 1.00 | 0.00 | 3A7 |
| ATOM | 642 | CA | ASP | 123 | 29.236 | −38.402 | 7.576 | 1.00 | 0.00 | 3A7 |
| ATOM | 643 | CB | ASP | 123 | 27.977 | −39.195 | 7.107 | 1.00 | 0.00 | 3A7 |
| ATOM | 644 | CG | ASP | 123 | 28.012 | −39.602 | 5.628 | 1.00 | 0.00 | 3A7 |
| ATOM | 645 | OD1 | ASP | 123 | 28.196 | −38.707 | 4.763 | 1.00 | 0.00 | 3A7 |
| ATOM | 646 | OD2 | ASP | 123 | 27.861 | −40.822 | 5.354 | 1.00 | 0.00 | 3A7 |
| ATOM | 647 | C | ASP | 123 | 30.485 | −39.259 | 7.471 | 1.00 | 0.00 | 3A7 |
| ATOM | 648 | O | ASP | 123 | 30.724 | −39.955 | 6.485 | 1.00 | 0.00 | 3A7 |
| ATOM | 649 | N | GLU | 124 | 31.302 | −39.189 | 8.540 | 1.00 | 0.00 | 3A7 |
| ATOM | 650 | CA | GLU | 124 | 32.560 | −39.870 | 8.683 | 1.00 | 0.00 | 3A7 |
| ATOM | 651 | CB | GLU | 124 | 33.626 | −39.519 | 7.609 | 1.00 | 0.00 | 3A7 |
| ATOM | 652 | CG | GLU | 124 | 34.965 | −40.257 | 7.793 | 1.00 | 0.00 | 3A7 |
| ATOM | 653 | CD | GLU | 124 | 35.918 | −39.851 | 6.672 | 1.00 | 0.00 | 3A7 |
| ATOM | 654 | OE1 | GLU | 124 | 36.993 | −39.275 | 6.989 | 1.00 | 0.00 | 3A7 |
| ATOM | 655 | OE2 | GLU | 124 | 35.585 | −40.115 | 5.486 | 1.00 | 0.00 | 3A7 |
| ATOM | 656 | C | GLU | 124 | 33.018 | −39.381 | 10.020 | 1.00 | 0.00 | 3A7 |
| ATOM | 657 | O | GLU | 124 | 33.474 | −40.149 | 10.866 | 1.00 | 0.00 | 3A7 |
| ATOM | 658 | N | GLU | 125 | 32.873 | −38.047 | 10.219 | 1.00 | 0.00 | 3A7 |
| ATOM | 659 | CA | GLU | 125 | 33.141 | −37.358 | 11.455 | 1.00 | 0.00 | 3A7 |
| ATOM | 660 | CB | GLU | 125 | 33.984 | −36.078 | 11.267 | 1.00 | 0.00 | 3A7 |
| ATOM | 661 | CG | GLU | 125 | 35.301 | −36.303 | 10.505 | 1.00 | 0.00 | 3A7 |
| ATOM | 662 | CD | GLU | 125 | 36.159 | −37.314 | 11.254 | 1.00 | 0.00 | 3A7 |
| ATOM | 663 | OE1 | GLU | 125 | 36.439 | −38.392 | 10.667 | 1.00 | 0.00 | 3A7 |
| ATOM | 664 | OE2 | GLU | 125 | 36.546 | −37.024 | 12.418 | 1.00 | 0.00 | 3A7 |
| ATOM | 665 | C | GLU | 125 | 31.844 | −36.969 | 12.096 | 1.00 | 0.00 | 3A7 |
| ATOM | 666 | O | GLU | 125 | 31.785 | −36.775 | 13.306 | 1.00 | 0.00 | 3A7 |
| ATOM | 667 | N | TRP | 126 | 30.739 | −36.843 | 11.315 | 1.00 | 0.00 | 3A7 |
| ATOM | 668 | CA | TRP | 126 | 29.448 | −36.458 | 11.855 | 1.00 | 0.00 | 3A7 |
| ATOM | 669 | CB | TRP | 126 | 28.460 | −36.001 | 10.762 | 1.00 | 0.00 | 3A7 |
| ATOM | 670 | CG | TRP | 126 | 27.427 | −34.955 | 11.159 | 1.00 | 0.00 | 3A7 |
| ATOM | 671 | CD2 | TRP | 126 | 27.726 | −33.684 | 11.776 | 1.00 | 0.00 | 3A7 |
| ATOM | 672 | CD1 | TRP | 126 | 26.092 | −34.933 | 10.869 | 1.00 | 0.00 | 3A7 |
| ATOM | 673 | NE1 | TRP | 126 | 25.536 | −33.741 | 11.271 | 1.00 | 0.00 | 3A7 |
| ATOM | 674 | CE2 | TRP | 126 | 26.527 | −32.954 | 11.818 | 1.00 | 0.00 | 3A7 |
| ATOM | 675 | CE3 | TRP | 126 | 28.905 | −33.139 | 12.252 | 1.00 | 0.00 | 3A7 |
| ATOM | 676 | CZ2 | TRP | 126 | 26.494 | −31.670 | 12.329 | 1.00 | 0.00 | 3A7 |
| ATOM | 677 | CZ3 | TRP | 126 | 28.868 | −31.850 | 12.774 | 1.00 | 0.00 | 3A7 |
| ATOM | 678 | CH2 | TRP | 126 | 27.680 | −31.123 | 12.810 | 1.00 | 0.00 | 3A7 |
| ATOM | 679 | C | TRP | 126 | 28.874 | −37.602 | 12.636 | 1.00 | 0.00 | 3A7 |
| ATOM | 680 | O | TRP | 126 | 28.393 | −37.423 | 13.750 | 1.00 | 0.00 | 3A7 |
| ATOM | 681 | N | LYS | 127 | 28.962 | −38.842 | 12.090 | 1.00 | 0.00 | 3A7 |
| ATOM | 682 | CA | LYS | 127 | 28.511 | −40.060 | 12.743 | 1.00 | 0.00 | 3A7 |
| ATOM | 683 | CB | LYS | 127 | 28.749 | −41.279 | 11.833 | 1.00 | 0.00 | 3A7 |
| ATOM | 684 | CG | LYS | 127 | 28.086 | −42.581 | 12.315 | 1.00 | 0.00 | 3A7 |
| ATOM | 685 | CD | LYS | 127 | 28.264 | −43.736 | 11.316 | 1.00 | 0.00 | 3A7 |
| ATOM | 686 | CE | LYS | 127 | 27.607 | −45.046 | 11.770 | 1.00 | 0.00 | 3A7 |
| ATOM | 687 | NZ | LYS | 127 | 26.139 | −44.893 | 11.883 | 1.00 | 0.00 | 3A7 |
| ATOM | 688 | C | LYS | 127 | 29.196 | −40.320 | 14.072 | 1.00 | 0.00 | 3A7 |
| ATOM | 689 | O | LYS | 127 | 28.561 | −40.651 | 15.070 | 1.00 | 0.00 | 3A7 |
| ATOM | 690 | N | ARG | 128 | 30.530 | −40.112 | 14.123 | 1.00 | 0.00 | 3A7 |
| ATOM | 691 | CA | ARG | 128 | 31.301 | −40.271 | 15.331 | 1.00 | 0.00 | 3A7 |
| ATOM | 692 | CB | ARG | 128 | 32.812 | −40.274 | 15.061 | 1.00 | 0.00 | 3A7 |
| ATOM | 693 | CG | ARG | 128 | 33.252 | −41.515 | 14.267 | 1.00 | 0.00 | 3A7 |
| ATOM | 694 | CD | ARG | 128 | 34.765 | −41.777 | 14.317 | 1.00 | 0.00 | 3A7 |
| ATOM | 695 | NE | ARG | 128 | 35.198 | −41.942 | 15.749 | 1.00 | 0.00 | 3A7 |
| ATOM | 696 | CZ | ARG | 128 | 34.997 | −43.090 | 16.469 | 1.00 | 0.00 | 3A7 |
| ATOM | 697 | NH1 | ARG | 128 | 35.389 | −43.140 | 17.776 | 1.00 | 0.00 | 3A7 |
| ATOM | 698 | NH2 | ARG | 128 | 34.422 | −44.186 | 15.894 | 1.00 | 0.00 | 3A7 |
| ATOM | 699 | C | ARG | 128 | 30.991 | −39.217 | 16.367 | 1.00 | 0.00 | 3A7 |
| ATOM | 700 | O | ARG | 128 | 30.847 | −39.549 | 17.535 | 1.00 | 0.00 | 3A7 |
| ATOM | 701 | N | ILE | 129 | 30.823 | −37.931 | 15.976 | 1.00 | 0.00 | 3A7 |
| ATOM | 702 | CA | ILE | 129 | 30.486 | −36.852 | 16.897 | 1.00 | 0.00 | 3A7 |
| ATOM | 703 | CB | ILE | 129 | 30.651 | −35.498 | 16.229 | 1.00 | 0.00 | 3A7 |
| ATOM | 704 | CG2 | ILE | 129 | 29.684 | −34.398 | 16.740 | 1.00 | 0.00 | 3A7 |
| ATOM | 705 | CG1 | ILE | 129 | 32.104 | −34.975 | 16.405 | 1.00 | 0.00 | 3A7 |
| ATOM | 706 | CD | ILE | 129 | 33.250 | −35.944 | 16.093 | 1.00 | 0.00 | 3A7 |
| ATOM | 707 | C | ILE | 129 | 29.095 | −37.028 | 17.444 | 1.00 | 0.00 | 3A7 |
| ATOM | 708 | O | ILE | 129 | 28.882 | −36.896 | 18.640 | 1.00 | 0.00 | 3A7 |
| ATOM | 709 | N | ARG | 130 | 28.114 | −37.398 | 16.595 | 1.00 | 0.00 | 3A7 |
| ATOM | 710 | CA | ARG | 130 | 26.759 | −37.661 | 17.021 | 1.00 | 0.00 | 3A7 |
| ATOM | 711 | CB | ARG | 130 | 25.849 | −37.943 | 15.811 | 1.00 | 0.00 | 3A7 |
| ATOM | 712 | CG | ARG | 130 | 25.540 | −36.681 | 14.987 | 1.00 | 0.00 | 3A7 |
| ATOM | 713 | CD | ARG | 130 | 24.607 | −36.942 | 13.796 | 1.00 | 0.00 | 3A7 |
| ATOM | 714 | NE | ARG | 130 | 25.280 | −37.896 | 12.857 | 1.00 | 0.00 | 3A7 |
| ATOM | 715 | CZ | ARG | 130 | 24.691 | −38.311 | 11.694 | 1.00 | 0.00 | 3A7 |
| ATOM | 716 | NH1 | ARG | 130 | 25.358 | −39.169 | 10.868 | 1.00 | 0.00 | 3A7 |
| ATOM | 717 | NH2 | ARG | 130 | 23.446 | −37.869 | 11.351 | 1.00 | 0.00 | 3A7 |
| ATOM | 718 | C | ARG | 130 | 26.695 | −38.843 | 17.970 | 1.00 | 0.00 | 3A7 |
| ATOM | 719 | O | ARG | 130 | 26.037 | −38.756 | 18.996 | 1.00 | 0.00 | 3A7 |
| ATOM | 720 | N | SER | 131 | 27.443 | −39.943 | 17.701 | 1.00 | 0.00 | 3A7 |
| ATOM | 721 | CA | SER | 131 | 27.492 | −41.105 | 18.571 | 1.00 | 0.00 | 3A7 |
| ATOM | 722 | CB | SER | 131 | 28.322 | −42.268 | 17.985 | 1.00 | 0.00 | 3A7 |
| ATOM | 723 | OG | SER | 131 | 29.697 | −41.986 | 17.760 | 1.00 | 0.00 | 3A7 |
| ATOM | 724 | C | SER | 131 | 28.053 | −40.816 | 19.936 | 1.00 | 0.00 | 3A7 |
| ATOM | 725 | O | SER | 131 | 27.615 | −41.352 | 20.947 | 1.00 | 0.00 | 3A7 |
| ATOM | 726 | N | LEU | 132 | 29.066 | −39.936 | 19.998 | 1.00 | 0.00 | 3A7 |
| ATOM | 727 | CA | LEU | 132 | 29.705 | −39.583 | 21.240 | 1.00 | 0.00 | 3A7 |
| ATOM | 728 | CB | LEU | 132 | 31.017 | −38.838 | 20.968 | 1.00 | 0.00 | 3A7 |
| ATOM | 729 | CG | LEU | 132 | 32.105 | −39.732 | 20.333 | 1.00 | 0.00 | 3A7 |
| ATOM | 730 | CD1 | LEU | 132 | 33.135 | −38.875 | 19.583 | 1.00 | 0.00 | 3A7 |
| ATOM | 731 | CD2 | LEU | 132 | 32.782 | −40.648 | 21.366 | 1.00 | 0.00 | 3A7 |
| ATOM | 732 | C | LEU | 132 | 28.808 | −38.721 | 22.089 | 1.00 | 0.00 | 3A7 |
| ATOM | 733 | O | LEU | 132 | 28.688 | −38.937 | 23.289 | 1.00 | 0.00 | 3A7 |
| ATOM | 734 | N | LEU | 133 | 28.116 | −37.745 | 21.460 | 1.00 | 0.00 | 3A7 |
| ATOM | 735 | CA | LEU | 133 | 27.269 | −36.794 | 22.145 | 1.00 | 0.00 | 3A7 |
| ATOM | 736 | CB | LEU | 133 | 26.997 | −35.547 | 21.272 | 1.00 | 0.00 | 3A7 |
| ATOM | 737 | CG | LEU | 133 | 28.266 | −34.787 | 20.869 | 1.00 | 0.00 | 3A7 |
| ATOM | 738 | CD1 | LEU | 133 | 27.918 | −33.493 | 20.117 | 1.00 | 0.00 | 3A7 |
| ATOM | 739 | CD2 | LEU | 133 | 29.211 | −34.589 | 22.052 | 1.00 | 0.00 | 3A7 |
| ATOM | 740 | C | LEU | 133 | 25.928 | −37.334 | 22.547 | 1.00 | 0.00 | 3A7 |
| ATOM | 741 | O | LEU | 133 | 25.403 | −36.955 | 23.590 | 1.00 | 0.00 | 3A7 |
| ATOM | 742 | N | SER | 134 | 25.333 | −38.237 | 21.738 | 1.00 | 0.00 | 3A7 |
| ATOM | 743 | CA | SER | 134 | 24.003 | −38.748 | 21.970 | 1.00 | 0.00 | 3A7 |
| ATOM | 744 | CB | SER | 134 | 23.511 | −39.662 | 20.844 | 1.00 | 0.00 | 3A7 |
| ATOM | 745 | OG | SER | 134 | 24.394 | −40.706 | 20.461 | 1.00 | 0.00 | 3A7 |
| ATOM | 746 | C | SER | 134 | 23.800 | −39.430 | 23.296 | 1.00 | 0.00 | 3A7 |
| ATOM | 747 | O | SER | 134 | 22.725 | −39.226 | 23.854 | 1.00 | 0.00 | 3A7 |
| ATOM | 748 | N | PRO | 135 | 24.715 | −40.198 | 23.891 | 1.00 | 0.00 | 3A7 |
| ATOM | 749 | CA | PRO | 135 | 24.491 | −40.773 | 25.190 | 1.00 | 0.00 | 3A7 |
| ATOM | 750 | CD | PRO | 135 | 25.854 | −40.834 | 23.260 | 1.00 | 0.00 | 3A7 |
| ATOM | 751 | CB | PRO | 135 | 25.551 | −41.871 | 25.355 | 1.00 | 0.00 | 3A7 |
| ATOM | 752 | CG | PRO | 135 | 25.961 | −42.203 | 23.924 | 1.00 | 0.00 | 3A7 |
| ATOM | 753 | C | PRO | 135 | 24.618 | −39.783 | 26.310 | 1.00 | 0.00 | 3A7 |
| ATOM | 754 | O | PRO | 135 | 23.867 | −39.912 | 27.273 | 1.00 | 0.00 | 3A7 |
| ATOM | 755 | N | THR | 136 | 25.553 | −38.805 | 26.231 | 1.00 | 0.00 | 3A7 |
| ATOM | 756 | CA | THR | 136 | 25.770 | −37.840 | 27.291 | 1.00 | 0.00 | 3A7 |
| ATOM | 757 | CB | THR | 136 | 27.148 | −37.210 | 27.207 | 1.00 | 0.00 | 3A7 |
| ATOM | 758 | OG1 | THR | 136 | 27.386 | −36.654 | 25.918 | 1.00 | 0.00 | 3A7 |
| ATOM | 759 | CG2 | THR | 136 | 28.182 | −38.319 | 27.492 | 1.00 | 0.00 | 3A7 |
| ATOM | 760 | C | THR | 136 | 24.693 | −36.769 | 27.320 | 1.00 | 0.00 | 3A7 |
| ATOM | 761 | O | THR | 136 | 24.369 | −36.212 | 28.360 | 1.00 | 0.00 | 3A7 |
| ATOM | 762 | N | PHE | 137 | 24.066 | −36.471 | 26.165 | 1.00 | 0.00 | 3A7 |
| ATOM | 763 | CA | PHE | 137 | 23.044 | −35.454 | 26.069 | 1.00 | 0.00 | 3A7 |
| ATOM | 764 | CB | PHE | 137 | 23.219 | −34.665 | 24.778 | 1.00 | 0.00 | 3A7 |
| ATOM | 765 | CG | PHE | 137 | 24.305 | −33.645 | 25.018 | 1.00 | 0.00 | 3A7 |
| ATOM | 766 | CD1 | PHE | 137 | 25.578 | −33.792 | 24.468 | 1.00 | 0.00 | 3A7 |
| ATOM | 767 | CD2 | PHE | 137 | 24.057 | −32.535 | 25.831 | 1.00 | 0.00 | 3A7 |
| ATOM | 768 | CE1 | PHE | 137 | 26.580 | −32.863 | 24.725 | 1.00 | 0.00 | 3A7 |
| ATOM | 769 | CE2 | PHE | 137 | 25.050 | −31.590 | 26.070 | 1.00 | 0.00 | 3A7 |
| ATOM | 770 | CZ | PHE | 137 | 26.316 | −31.757 | 25.524 | 1.00 | 0.00 | 3A7 |
| ATOM | 771 | C | PHE | 137 | 21.711 | −36.153 | 26.100 | 1.00 | 0.00 | 3A7 |
| ATOM | 772 | O | PHE | 137 | 20.779 | −35.799 | 25.406 | 1.00 | 0.00 | 3A7 |
| ATOM | 773 | N | THR | 138 | 21.542 | −37.179 | 26.952 | 1.00 | 0.00 | 3A7 |
| ATOM | 774 | CA | THR | 138 | 20.246 | −37.782 | 27.179 | 1.00 | 0.00 | 3A7 |
| ATOM | 775 | CB | THR | 138 | 20.313 | −39.259 | 27.505 | 1.00 | 0.00 | 3A7 |
| ATOM | 776 | OG1 | THR | 138 | 21.270 | −39.549 | 28.518 | 1.00 | 0.00 | 3A7 |
| ATOM | 777 | CG2 | THR | 138 | 20.673 | −40.035 | 26.226 | 1.00 | 0.00 | 3A7 |
| ATOM | 778 | C | THR | 138 | 19.588 | −37.063 | 28.326 | 1.00 | 0.00 | 3A7 |
| ATOM | 779 | O | THR | 138 | 20.234 | −36.412 | 29.149 | 1.00 | 0.00 | 3A7 |
| ATOM | 780 | N | SER | 139 | 18.250 | −37.211 | 28.416 | 1.00 | 0.00 | 3A7 |
| ATOM | 781 | CA | SER | 139 | 17.433 | −36.585 | 29.427 | 1.00 | 0.00 | 3A7 |
| ATOM | 782 | CB | SER | 139 | 15.917 | −36.809 | 29.183 | 1.00 | 0.00 | 3A7 |
| ATOM | 783 | OG | SER | 139 | 15.590 | −38.188 | 29.037 | 1.00 | 0.00 | 3A7 |
| ATOM | 784 | C | SER | 139 | 17.797 | −37.053 | 30.810 | 1.00 | 0.00 | 3A7 |
| ATOM | 785 | O | SER | 139 | 17.738 | −36.290 | 31.765 | 1.00 | 0.00 | 3A7 |
| ATOM | 786 | N | GLY | 140 | 18.263 | −38.318 | 30.922 | 1.00 | 0.00 | 3A7 |
| ATOM | 787 | CA | GLY | 140 | 18.713 | −38.898 | 32.158 | 1.00 | 0.00 | 3A7 |
| ATOM | 788 | C | GLY | 140 | 20.032 | −38.362 | 32.618 | 1.00 | 0.00 | 3A7 |
| ATOM | 789 | O | GLY | 140 | 20.273 | −38.260 | 33.820 | 1.00 | 0.00 | 3A7 |
| ATOM | 790 | N | LYS | 141 | 20.933 | −38.021 | 31.673 | 1.00 | 0.00 | 3A7 |
| ATOM | 791 | CA | LYS | 141 | 22.309 | −37.803 | 32.064 | 1.00 | 0.00 | 3A7 |
| ATOM | 792 | CB | LYS | 141 | 23.285 | −38.611 | 31.169 | 1.00 | 0.00 | 3A7 |
| ATOM | 793 | CG | LYS | 141 | 23.070 | −40.129 | 31.293 | 1.00 | 0.00 | 3A7 |
| ATOM | 794 | CD | LYS | 141 | 24.150 | −40.944 | 30.571 | 1.00 | 0.00 | 3A7 |
| ATOM | 795 | CE | LYS | 141 | 23.916 | −42.456 | 30.655 | 1.00 | 0.00 | 3A7 |
| ATOM | 796 | NZ | LYS | 141 | 24.978 | −43.189 | 29.928 | 1.00 | 0.00 | 3A7 |
| ATOM | 797 | C | LYS | 141 | 22.832 | −36.386 | 32.232 | 1.00 | 0.00 | 3A7 |
| ATOM | 798 | O | LYS | 141 | 22.859 | −35.680 | 33.265 | 1.00 | 0.00 | 3A7 |
| ATOM | 799 | N | LEU | 142 | 23.635 | −36.031 | 31.185 | 1.00 | 0.00 | 3A7 |
| ATOM | 800 | CA | LEU | 142 | 24.644 | −34.970 | 31.099 | 1.00 | 0.00 | 3A7 |
| ATOM | 801 | CB | LEU | 142 | 23.972 | −33.593 | 30.894 | 1.00 | 0.00 | 3A7 |
| ATOM | 802 | CG | LEU | 142 | 24.455 | −32.799 | 29.656 | 1.00 | 0.00 | 3A7 |
| ATOM | 803 | CD1 | LEU | 142 | 23.560 | −31.564 | 29.439 | 1.00 | 0.00 | 3A7 |
| ATOM | 804 | CD2 | LEU | 142 | 25.943 | −32.407 | 29.718 | 1.00 | 0.00 | 3A7 |
| ATOM | 805 | C | LEU | 142 | 25.663 | −34.903 | 32.241 | 1.00 | 0.00 | 3A7 |
| ATOM | 806 | O | LEU | 142 | 26.359 | −33.901 | 32.398 | 1.00 | 0.00 | 3A7 |
| ATOM | 807 | N | LYS | 143 | 25.729 | −35.975 | 33.085 | 1.00 | 0.00 | 3A7 |
| ATOM | 808 | CA | LYS | 143 | 26.391 | −36.077 | 34.381 | 1.00 | 0.00 | 3A7 |
| ATOM | 809 | CB | LYS | 143 | 27.931 | −35.966 | 34.315 | 1.00 | 0.00 | 3A7 |
| ATOM | 810 | CG | LYS | 143 | 28.567 | −37.003 | 33.373 | 1.00 | 0.00 | 3A7 |
| ATOM | 811 | CD | LYS | 143 | 28.188 | −38.480 | 33.614 | 1.00 | 0.00 | 3A7 |
| ATOM | 812 | CE | LYS | 143 | 28.784 | −39.129 | 34.873 | 1.00 | 0.00 | 3A7 |
| ATOM | 813 | NZ | LYS | 143 | 28.177 | −38.601 | 36.117 | 1.00 | 0.00 | 3A7 |
| ATOM | 814 | C | LYS | 143 | 25.831 | −35.060 | 35.351 | 1.00 | 0.00 | 3A7 |
| ATOM | 815 | O | LYS | 143 | 26.561 | −34.248 | 35.919 | 1.00 | 0.00 | 3A7 |
| ATOM | 816 | N | GLU | 144 | 24.476 | −35.097 | 35.479 | 1.00 | 0.00 | 3A7 |
| ATOM | 817 | CA | GLU | 144 | 23.593 | −34.122 | 36.090 | 1.00 | 0.00 | 3A7 |
| ATOM | 818 | CB | GLU | 144 | 24.058 | −33.575 | 37.466 | 1.00 | 0.00 | 3A7 |
| ATOM | 819 | CG | GLU | 144 | 24.205 | −34.669 | 38.535 | 1.00 | 0.00 | 3A7 |
| ATOM | 820 | CD | GLU | 144 | 24.730 | −34.037 | 39.820 | 1.00 | 0.00 | 3A7 |
| ATOM | 821 | OE1 | GLU | 144 | 25.849 | −34.422 | 40.255 | 1.00 | 0.00 | 3A7 |
| ATOM | 822 | OE2 | GLU | 144 | 24.022 | −33.161 | 40.384 | 1.00 | 0.00 | 3A7 |
| ATOM | 823 | C | GLU | 144 | 23.380 | −32.972 | 35.123 | 1.00 | 0.00 | 3A7 |
| ATOM | 824 | O | GLU | 144 | 24.284 | −32.166 | 34.907 | 1.00 | 0.00 | 3A7 |
| ATOM | 825 | N | MET | 145 | 22.163 | −32.867 | 34.523 | 1.00 | 0.00 | 3A7 |
| ATOM | 826 | CA | MET | 145 | 21.768 | −31.691 | 33.757 | 1.00 | 0.00 | 3A7 |
| ATOM | 827 | CB | MET | 145 | 21.203 | −31.937 | 32.347 | 1.00 | 0.00 | 3A7 |
| ATOM | 828 | CG | MET | 145 | 20.243 | −33.140 | 32.238 | 1.00 | 0.00 | 3A7 |
| ATOM | 829 | SD | MET | 145 | 19.496 | −33.350 | 30.593 | 1.00 | 0.00 | 3A7 |
| ATOM | 830 | CE | MET | 145 | 18.314 | −31.978 | 30.743 | 1.00 | 0.00 | 3A7 |
| ATOM | 831 | C | MET | 145 | 20.736 | −30.926 | 34.510 | 1.00 | 0.00 | 3A7 |
| ATOM | 832 | O | MET | 145 | 20.810 | −29.704 | 34.599 | 1.00 | 0.00 | 3A7 |
| ATOM | 833 | N | VAL | 146 | 19.718 | −31.609 | 35.082 | 1.00 | 0.00 | 3A7 |
| ATOM | 834 | CA | VAL | 146 | 18.618 | −30.943 | 35.756 | 1.00 | 0.00 | 3A7 |
| ATOM | 835 | CB | VAL | 146 | 17.494 | −31.869 | 36.146 | 1.00 | 0.00 | 3A7 |
| ATOM | 836 | CG1 | VAL | 146 | 16.290 | −30.982 | 36.556 | 1.00 | 0.00 | 3A7 |
| ATOM | 837 | CG2 | VAL | 146 | 17.145 | −32.759 | 34.935 | 1.00 | 0.00 | 3A7 |
| ATOM | 838 | C | VAL | 146 | 19.066 | −30.148 | 36.957 | 1.00 | 0.00 | 3A7 |
| ATOM | 839 | O | VAL | 146 | 18.633 | −29.004 | 37.094 | 1.00 | 0.00 | 3A7 |
| ATOM | 840 | N | PRO | 147 | 19.976 | −30.644 | 37.801 | 1.00 | 0.00 | 3A7 |
| ATOM | 841 | CA | PRO | 147 | 20.491 | −29.872 | 38.908 | 1.00 | 0.00 | 3A7 |
| ATOM | 842 | CD | PRO | 147 | 20.305 | −32.060 | 37.960 | 1.00 | 0.00 | 3A7 |
| ATOM | 843 | CB | PRO | 147 | 21.360 | −30.856 | 39.708 | 1.00 | 0.00 | 3A7 |
| ATOM | 844 | CG | PRO | 147 | 20.726 | −32.213 | 39.420 | 1.00 | 0.00 | 3A7 |
| ATOM | 845 | C | PRO | 147 | 21.286 | −28.672 | 38.470 | 1.00 | 0.00 | 3A7 |
| ATOM | 846 | O | PRO | 147 | 21.286 | −27.680 | 39.190 | 1.00 | 0.00 | 3A7 |
| ATOM | 847 | N | ILE | 148 | 21.943 | −28.722 | 37.294 | 1.00 | 0.00 | 3A7 |
| ATOM | 848 | CA | ILE | 148 | 22.723 | −27.631 | 36.762 | 1.00 | 0.00 | 3A7 |
| ATOM | 849 | CB | ILE | 148 | 23.664 | −28.114 | 35.675 | 1.00 | 0.00 | 3A7 |
| ATOM | 850 | CG2 | ILE | 148 | 24.341 | −26.923 | 34.952 | 1.00 | 0.00 | 3A7 |
| ATOM | 851 | CG1 | ILE | 148 | 24.796 | −29.020 | 36.211 | 1.00 | 0.00 | 3A7 |
| ATOM | 852 | CD | ILE | 148 | 24.460 | −30.116 | 37.225 | 1.00 | 0.00 | 3A7 |
| ATOM | 853 | C | ILE | 148 | 21.813 | −26.558 | 36.204 | 1.00 | 0.00 | 3A7 |
| ATOM | 854 | O | ILE | 148 | 22.073 | −25.361 | 36.322 | 1.00 | 0.00 | 3A7 |
| ATOM | 855 | N | ILE | 149 | 20.672 | −26.968 | 35.607 | 1.00 | 0.00 | 3A7 |
| ATOM | 856 | CA | ILE | 149 | 19.682 | −26.045 | 35.098 | 1.00 | 0.00 | 3A7 |
| ATOM | 857 | CB | ILE | 149 | 18.645 | −26.744 | 34.263 | 1.00 | 0.00 | 3A7 |
| ATOM | 858 | CG2 | ILE | 149 | 17.595 | −25.733 | 33.743 | 1.00 | 0.00 | 3A7 |
| ATOM | 859 | CG1 | ILE | 149 | 19.377 | −27.390 | 33.058 | 1.00 | 0.00 | 3A7 |
| ATOM | 860 | CD | ILE | 149 | 18.552 | −28.444 | 32.319 | 1.00 | 0.00 | 3A7 |
| ATOM | 861 | C | ILE | 149 | 19.032 | −25.338 | 36.240 | 1.00 | 0.00 | 3A7 |
| ATOM | 862 | O | ILE | 149 | 18.779 | −24.137 | 36.179 | 1.00 | 0.00 | 3A7 |
| ATOM | 863 | N | ALA | 150 | 18.793 | −26.059 | 37.357 | 1.00 | 0.00 | 3A7 |
| ATOM | 864 | CA | ALA | 150 | 18.229 | −25.493 | 38.558 | 1.00 | 0.00 | 3A7 |
| ATOM | 865 | CB | ALA | 150 | 17.894 | −26.586 | 39.578 | 1.00 | 0.00 | 3A7 |
| ATOM | 866 | C | ALA | 150 | 19.160 | −24.490 | 39.195 | 1.00 | 0.00 | 3A7 |
| ATOM | 867 | O | ALA | 150 | 18.723 | −23.461 | 39.693 | 1.00 | 0.00 | 3A7 |
| ATOM | 868 | N | GLN | 151 | 20.488 | −24.737 | 39.144 | 1.00 | 0.00 | 3A7 |
| ATOM | 869 | CA | GLN | 151 | 21.492 | −23.836 | 39.661 | 1.00 | 0.00 | 3A7 |
| ATOM | 870 | CB | GLN | 151 | 22.910 | −24.428 | 39.555 | 1.00 | 0.00 | 3A7 |
| ATOM | 871 | CG | GLN | 151 | 23.192 | −25.483 | 40.637 | 1.00 | 0.00 | 3A7 |
| ATOM | 872 | CD | GLN | 151 | 24.551 | −26.129 | 40.357 | 1.00 | 0.00 | 3A7 |
| ATOM | 873 | OE1 | GLN | 151 | 25.580 | −25.444 | 40.306 | 1.00 | 0.00 | 3A7 |
| ATOM | 874 | NE2 | GLN | 151 | 24.541 | −27.485 | 40.172 | 1.00 | 0.00 | 3A7 |
| ATOM | 875 | C | GLN | 151 | 21.475 | −22.493 | 38.989 | 1.00 | 0.00 | 3A7 |
| ATOM | 876 | O | GLN | 151 | 21.418 | −21.447 | 39.634 | 1.00 | 0.00 | 3A7 |
| ATOM | 877 | N | TYR | 152 | 21.474 | −22.502 | 37.644 | 1.00 | 0.00 | 3A7 |
| ATOM | 878 | CA | TYR | 152 | 21.438 | −21.288 | 36.872 | 1.00 | 0.00 | 3A7 |
| ATOM | 879 | CB | TYR | 152 | 21.651 | −21.508 | 35.367 | 1.00 | 0.00 | 3A7 |
| ATOM | 880 | CG | TYR | 152 | 22.236 | −20.216 | 34.776 | 1.00 | 0.00 | 3A7 |
| ATOM | 881 | CD1 | TYR | 152 | 23.578 | −19.994 | 35.099 | 1.00 | 0.00 | 3A7 |
| ATOM | 882 | CD2 | TYR | 152 | 21.411 | −19.109 | 34.632 | 1.00 | 0.00 | 3A7 |
| ATOM | 883 | CE1 | TYR | 152 | 24.053 | −18.705 | 35.335 | 1.00 | 0.00 | 3A7 |
| ATOM | 884 | CE2 | TYR | 152 | 21.870 | −17.822 | 34.898 | 1.00 | 0.00 | 3A7 |
| ATOM | 885 | CZ | TYR | 152 | 23.208 | −17.612 | 35.201 | 1.00 | 0.00 | 3A7 |
| ATOM | 886 | OH | TYR | 152 | 23.690 | −16.302 | 35.411 | 1.00 | 0.00 | 3A7 |
| ATOM | 887 | C | TYR | 152 | 20.140 | −20.556 | 37.038 | 1.00 | 0.00 | 3A7 |
| ATOM | 888 | O | TYR | 152 | 20.108 | −19.335 | 37.134 | 1.00 | 0.00 | 3A7 |
| ATOM | 889 | N | GLY | 153 | 19.025 | −21.301 | 37.117 | 1.00 | 0.00 | 3A7 |
| ATOM | 890 | CA | GLY | 153 | 17.715 | −20.733 | 37.311 | 1.00 | 0.00 | 3A7 |
| ATOM | 891 | C | GLY | 153 | 17.596 | −19.972 | 38.591 | 1.00 | 0.00 | 3A7 |
| ATOM | 892 | O | GLY | 153 | 16.977 | −18.915 | 38.657 | 1.00 | 0.00 | 3A7 |
| ATOM | 893 | N | ASP | 154 | 18.270 | −20.457 | 39.650 | 1.00 | 0.00 | 3A7 |
| ATOM | 894 | CA | ASP | 154 | 18.353 | −19.782 | 40.919 | 1.00 | 0.00 | 3A7 |
| ATOM | 895 | CB | ASP | 154 | 19.017 | −20.685 | 41.976 | 1.00 | 0.00 | 3A7 |
| ATOM | 896 | CG | ASP | 154 | 18.142 | −21.901 | 42.299 | 1.00 | 0.00 | 3A7 |
| ATOM | 897 | OD1 | ASP | 154 | 16.975 | −21.960 | 41.830 | 1.00 | 0.00 | 3A7 |
| ATOM | 898 | OD2 | ASP | 154 | 18.643 | −22.790 | 43.039 | 1.00 | 0.00 | 3A7 |
| ATOM | 899 | C | ASP | 154 | 19.130 | −18.483 | 40.808 | 1.00 | 0.00 | 3A7 |
| ATOM | 900 | O | ASP | 154 | 18.726 | −17.469 | 41.361 | 1.00 | 0.00 | 3A7 |
| ATOM | 901 | N | VAL | 155 | 20.243 | −18.474 | 40.041 | 1.00 | 0.00 | 3A7 |
| ATOM | 902 | CA | VAL | 155 | 21.039 | −17.293 | 39.761 | 1.00 | 0.00 | 3A7 |
| ATOM | 903 | CB | VAL | 155 | 22.308 | −17.637 | 38.989 | 1.00 | 0.00 | 3A7 |
| ATOM | 904 | CG1 | VAL | 155 | 23.103 | −16.370 | 38.590 | 1.00 | 0.00 | 3A7 |
| ATOM | 905 | CG2 | VAL | 155 | 23.251 | −18.537 | 39.870 | 1.00 | 0.00 | 3A7 |
| ATOM | 906 | C | VAL | 155 | 20.273 | −16.247 | 39.005 | 1.00 | 0.00 | 3A7 |
| ATOM | 907 | O | VAL | 155 | 20.407 | −15.062 | 39.286 | 1.00 | 0.00 | 3A7 |
| ATOM | 908 | N | LEU | 156 | 19.427 | −16.653 | 38.035 | 1.00 | 0.00 | 3A7 |
| ATOM | 909 | CA | LEU | 156 | 18.566 | −15.759 | 37.295 | 1.00 | 0.00 | 3A7 |
| ATOM | 910 | CB | LEU | 156 | 17.791 | −16.496 | 36.208 | 1.00 | 0.00 | 3A7 |
| ATOM | 911 | CG | LEU | 156 | 17.040 | −15.502 | 35.295 | 1.00 | 0.00 | 3A7 |
| ATOM | 912 | CD1 | LEU | 156 | 17.539 | −15.527 | 33.841 | 1.00 | 0.00 | 3A7 |
| ATOM | 913 | CD2 | LEU | 156 | 15.520 | −15.740 | 35.345 | 1.00 | 0.00 | 3A7 |
| ATOM | 914 | C | LEU | 156 | 17.570 | −15.040 | 38.134 | 1.00 | 0.00 | 3A7 |
| ATOM | 915 | O | LEU | 156 | 17.442 | −13.817 | 38.081 | 1.00 | 0.00 | 3A7 |
| ATOM | 916 | N | VAL | 157 | 16.840 | −15.805 | 38.969 | 1.00 | 0.00 | 3A7 |
| ATOM | 917 | CA | VAL | 157 | 15.830 | −15.290 | 39.861 | 1.00 | 0.00 | 3A7 |
| ATOM | 918 | CB | VAL | 157 | 15.164 | −16.419 | 40.629 | 1.00 | 0.00 | 3A7 |
| ATOM | 919 | CG1 | VAL | 157 | 14.204 | −15.903 | 41.728 | 1.00 | 0.00 | 3A7 |
| ATOM | 920 | CG2 | VAL | 157 | 14.373 | −17.263 | 39.609 | 1.00 | 0.00 | 3A7 |
| ATOM | 921 | C | VAL | 157 | 16.437 | −14.314 | 40.832 | 1.00 | 0.00 | 3A7 |
| ATOM | 922 | O | VAL | 157 | 15.906 | −13.236 | 41.069 | 1.00 | 0.00 | 3A7 |
| ATOM | 923 | N | ARG | 158 | 17.626 | −14.668 | 41.358 | 1.00 | 0.00 | 3A7 |
| ATOM | 924 | CA | ARG | 158 | 18.365 | −13.822 | 42.251 | 1.00 | 0.00 | 3A7 |
| ATOM | 925 | CB | ARG | 158 | 19.590 | −14.557 | 42.789 | 1.00 | 0.00 | 3A7 |
| ATOM | 926 | CG | ARG | 158 | 20.308 | −13.863 | 43.961 | 1.00 | 0.00 | 3A7 |
| ATOM | 927 | CD | ARG | 158 | 21.475 | −14.689 | 44.519 | 1.00 | 0.00 | 3A7 |
| ATOM | 928 | NE | ARG | 158 | 20.932 | −15.988 | 45.038 | 1.00 | 0.00 | 3A7 |
| ATOM | 929 | CZ | ARG | 158 | 21.742 | −17.042 | 45.358 | 1.00 | 0.00 | 3A7 |
| ATOM | 930 | NH1 | ARG | 158 | 21.187 | −18.206 | 45.808 | 1.00 | 0.00 | 3A7 |
| ATOM | 931 | NH2 | ARG | 158 | 23.097 | −16.943 | 45.230 | 1.00 | 0.00 | 3A7 |
| ATOM | 932 | C | ARG | 158 | 18.810 | −12.537 | 41.638 | 1.00 | 0.00 | 3A7 |
| ATOM | 933 | O | ARG | 158 | 18.732 | −11.498 | 42.274 | 1.00 | 0.00 | 3A7 |
| ATOM | 934 | N | ASN | 159 | 19.237 | −12.550 | 40.359 | 1.00 | 0.00 | 3A7 |
| ATOM | 935 | CA | ASN | 159 | 19.621 | −11.357 | 39.637 | 1.00 | 0.00 | 3A7 |
| ATOM | 936 | CB | ASN | 159 | 20.171 | −11.697 | 38.250 | 1.00 | 0.00 | 3A7 |
| ATOM | 937 | CG | ASN | 159 | 21.490 | −12.475 | 38.358 | 1.00 | 0.00 | 3A7 |
| ATOM | 938 | OD1 | ASN | 159 | 22.072 | −12.627 | 39.439 | 1.00 | 0.00 | 3A7 |
| ATOM | 939 | ND2 | ASN | 159 | 21.964 | −12.980 | 37.177 | 1.00 | 0.00 | 3A7 |
| ATOM | 940 | C | ASN | 159 | 18.466 | −10.408 | 39.454 | 1.00 | 0.00 | 3A7 |
| ATOM | 941 | O | ASN | 159 | 18.606 | −9.195 | 39.568 | 1.00 | 0.00 | 3A7 |
| ATOM | 942 | N | LEU | 160 | 17.267 | −10.966 | 39.205 | 1.00 | 0.00 | 3A7 |
| ATOM | 943 | CA | LEU | 160 | 16.066 | −10.198 | 39.031 | 1.00 | 0.00 | 3A7 |
| ATOM | 944 | CB | LEU | 160 | 14.972 | −11.061 | 38.383 | 1.00 | 0.00 | 3A7 |
| ATOM | 945 | CG | LEU | 160 | 15.234 | −11.457 | 36.902 | 1.00 | 0.00 | 3A7 |
| ATOM | 946 | CD1 | LEU | 160 | 14.149 | −12.430 | 36.402 | 1.00 | 0.00 | 3A7 |
| ATOM | 947 | CD2 | LEU | 160 | 15.359 | −10.246 | 35.959 | 1.00 | 0.00 | 3A7 |
| ATOM | 948 | C | LEU | 160 | 15.557 | −9.655 | 40.349 | 1.00 | 0.00 | 3A7 |
| ATOM | 949 | O | LEU | 160 | 14.965 | −8.580 | 40.420 | 1.00 | 0.00 | 3A7 |
| ATOM | 950 | N | ARG | 161 | 15.836 | −10.377 | 41.455 | 1.00 | 0.00 | 3A7 |
| ATOM | 951 | CA | ARG | 161 | 15.487 | −9.958 | 42.787 | 1.00 | 0.00 | 3A7 |
| ATOM | 952 | CB | ARG | 161 | 15.773 | −11.052 | 43.830 | 1.00 | 0.00 | 3A7 |
| ATOM | 953 | CG | ARG | 161 | 14.640 | −12.097 | 43.887 | 1.00 | 0.00 | 3A7 |
| ATOM | 954 | CD | ARG | 161 | 14.752 | −13.097 | 45.047 | 1.00 | 0.00 | 3A7 |
| ATOM | 955 | NE | ARG | 161 | 15.964 | −13.954 | 44.841 | 1.00 | 0.00 | 3A7 |
| ATOM | 956 | CZ | ARG | 161 | 16.221 | −15.051 | 45.617 | 1.00 | 0.00 | 3A7 |
| ATOM | 957 | NH1 | ARG | 161 | 17.333 | −15.804 | 45.374 | 1.00 | 0.00 | 3A7 |
| ATOM | 958 | NH2 | ARG | 161 | 15.375 | −15.400 | 46.629 | 1.00 | 0.00 | 3A7 |
| ATOM | 959 | C | ARG | 161 | 16.228 | −8.712 | 43.210 | 1.00 | 0.00 | 3A7 |
| ATOM | 960 | O | ARG | 161 | 15.673 | −7.843 | 43.879 | 1.00 | 0.00 | 3A7 |
| ATOM | 961 | N | ARG | 162 | 17.510 | −8.597 | 42.792 | 1.00 | 0.00 | 3A7 |
| ATOM | 962 | CA | ARG | 162 | 18.380 | −7.499 | 43.134 | 1.00 | 0.00 | 3A7 |
| ATOM | 963 | CB | ARG | 162 | 19.851 | −7.869 | 42.894 | 1.00 | 0.00 | 3A7 |
| ATOM | 964 | CG | ARG | 162 | 20.346 | −8.986 | 43.829 | 1.00 | 0.00 | 3A7 |
| ATOM | 965 | CD | ARG | 162 | 21.838 | −9.302 | 43.666 | 1.00 | 0.00 | 3A7 |
| ATOM | 966 | NE | ARG | 162 | 22.095 | −9.696 | 42.242 | 1.00 | 0.00 | 3A7 |
| ATOM | 967 | CZ | ARG | 162 | 23.363 | −9.855 | 41.755 | 1.00 | 0.00 | 3A7 |
| ATOM | 968 | NH1 | ARG | 162 | 23.548 | −10.141 | 40.433 | 1.00 | 0.00 | 3A7 |
| ATOM | 969 | NH2 | ARG | 162 | 24.444 | −9.724 | 42.577 | 1.00 | 0.00 | 3A7 |
| ATOM | 970 | C | ARG | 162 | 18.080 | −6.236 | 42.355 | 1.00 | 0.00 | 3A7 |
| ATOM | 971 | O | ARG | 162 | 18.755 | −5.220 | 42.500 | 1.00 | 0.00 | 3A7 |
| ATOM | 972 | N | GLU | 163 | 17.014 | −6.247 | 41.519 | 1.00 | 0.00 | 3A7 |
| ATOM | 973 | CA | GLU | 163 | 16.615 | −5.103 | 40.744 | 1.00 | 0.00 | 3A7 |
| ATOM | 974 | CB | GLU | 163 | 15.965 | −5.504 | 39.413 | 1.00 | 0.00 | 3A7 |
| ATOM | 975 | CG | GLU | 163 | 16.981 | −6.330 | 38.605 | 1.00 | 0.00 | 3A7 |
| ATOM | 976 | CD | GLU | 163 | 16.426 | −6.716 | 37.246 | 1.00 | 0.00 | 3A7 |
| ATOM | 977 | OE1 | GLU | 163 | 17.080 | −6.372 | 36.227 | 1.00 | 0.00 | 3A7 |
| ATOM | 978 | OE2 | GLU | 163 | 15.354 | −7.376 | 37.210 | 1.00 | 0.00 | 3A7 |
| ATOM | 979 | C | GLU | 163 | 15.784 | −4.183 | 41.582 | 1.00 | 0.00 | 3A7 |
| ATOM | 980 | O | GLU | 163 | 15.662 | −2.998 | 41.288 | 1.00 | 0.00 | 3A7 |
| ATOM | 981 | N | ALA | 164 | 15.230 | −4.708 | 42.699 | 1.00 | 0.00 | 3A7 |
| ATOM | 982 | CA | ALA | 164 | 14.728 | −3.939 | 43.812 | 1.00 | 0.00 | 3A7 |
| ATOM | 983 | CB | ALA | 164 | 15.779 | −2.963 | 44.394 | 1.00 | 0.00 | 3A7 |
| ATOM | 984 | C | ALA | 164 | 13.480 | −3.200 | 43.529 | 1.00 | 0.00 | 3A7 |
| ATOM | 985 | O | ALA | 164 | 13.505 | −1.993 | 43.644 | 1.00 | 0.00 | 3A7 |
| ATOM | 986 | N | GLU | 165 | 12.363 | −3.878 | 43.176 | 1.00 | 0.00 | 3A7 |
| ATOM | 987 | CA | GLU | 165 | 10.993 | −3.400 | 43.222 | 1.00 | 0.00 | 3A7 |
| ATOM | 988 | CB | GLU | 165 | 10.561 | −2.688 | 44.540 | 1.00 | 0.00 | 3A7 |
| ATOM | 989 | CG | GLU | 165 | 10.851 | −3.510 | 45.809 | 1.00 | 0.00 | 3A7 |
| ATOM | 990 | CD | GLU | 165 | 10.308 | −2.803 | 47.050 | 1.00 | 0.00 | 3A7 |
| ATOM | 991 | OE1 | GLU | 165 | 9.757 | −1.678 | 46.915 | 1.00 | 0.00 | 3A7 |
| ATOM | 992 | OE2 | GLU | 165 | 10.441 | −3.390 | 48.158 | 1.00 | 0.00 | 3A7 |
| ATOM | 993 | C | GLU | 165 | 10.541 | −2.574 | 42.031 | 1.00 | 0.00 | 3A7 |
| ATOM | 994 | O | GLU | 165 | 9.403 | −2.728 | 41.595 | 1.00 | 0.00 | 3A7 |
| ATOM | 995 | N | THR | 166 | 11.382 | −1.661 | 41.488 | 1.00 | 0.00 | 3A7 |
| ATOM | 996 | CA | THR | 166 | 10.979 | −0.758 | 40.425 | 1.00 | 0.00 | 3A7 |
| ATOM | 997 | CB | THR | 166 | 10.351 | 0.509 | 40.932 | 1.00 | 0.00 | 3A7 |
| ATOM | 998 | OG1 | THR | 166 | 9.175 | 0.217 | 41.676 | 1.00 | 0.00 | 3A7 |
| ATOM | 999 | CG2 | THR | 166 | 9.944 | 1.442 | 39.767 | 1.00 | 0.00 | 3A7 |
| ATOM | 1000 | C | THR | 166 | 12.235 | −0.374 | 39.728 | 1.00 | 0.00 | 3A7 |
| ATOM | 1001 | O | THR | 166 | 12.410 | −0.652 | 38.547 | 1.00 | 0.00 | 3A7 |
| ATOM | 1002 | N | GLY | 167 | 13.141 | 0.230 | 40.560 | 1.00 | 0.00 | 3A7 |
| ATOM | 1003 | CA | GLY | 167 | 14.556 | 0.524 | 40.415 | 1.00 | 0.00 | 3A7 |
| ATOM | 1004 | C | GLY | 167 | 14.980 | 0.965 | 39.058 | 1.00 | 0.00 | 3A7 |
| ATOM | 1005 | O | GLY | 167 | 15.037 | 2.155 | 38.750 | 1.00 | 0.00 | 3A7 |
| ATOM | 1006 | N | LYS | 168 | 15.246 | −0.048 | 38.208 | 1.00 | 0.00 | 3A7 |
| ATOM | 1007 | CA | LYS | 168 | 15.423 | 0.121 | 36.801 | 1.00 | 0.00 | 3A7 |
| ATOM | 1008 | CB | LYS | 168 | 16.887 | −0.172 | 36.380 | 1.00 | 0.00 | 3A7 |
| ATOM | 1009 | CG | LYS | 168 | 17.309 | 0.475 | 35.050 | 1.00 | 0.00 | 3A7 |
| ATOM | 1010 | CD | LYS | 168 | 18.803 | 0.294 | 34.725 | 1.00 | 0.00 | 3A7 |
| ATOM | 1011 | CE | LYS | 168 | 19.236 | −1.160 | 34.483 | 1.00 | 0.00 | 3A7 |
| ATOM | 1012 | NZ | LYS | 168 | 18.558 | −1.731 | 33.297 | 1.00 | 0.00 | 3A7 |
| ATOM | 1013 | C | LYS | 168 | 14.438 | −0.857 | 36.181 | 1.00 | 0.00 | 3A7 |
| ATOM | 1014 | O | LYS | 168 | 14.315 | −1.969 | 36.694 | 1.00 | 0.00 | 3A7 |
| ATOM | 1015 | N | PRO | 169 | 13.751 | −0.544 | 35.061 | 1.00 | 0.00 | 3A7 |
| ATOM | 1016 | CA | PRO | 169 | 13.228 | −1.488 | 34.077 | 1.00 | 0.00 | 3A7 |
| ATOM | 1017 | CD | PRO | 169 | 13.706 | 0.834 | 34.565 | 1.00 | 0.00 | 3A7 |
| ATOM | 1018 | CB | PRO | 169 | 12.639 | −0.568 | 33.005 | 1.00 | 0.00 | 3A7 |
| ATOM | 1019 | CG | PRO | 169 | 13.438 | 0.725 | 33.072 | 1.00 | 0.00 | 3A7 |
| ATOM | 1020 | C | PRO | 169 | 14.386 | −2.289 | 33.508 | 1.00 | 0.00 | 3A7 |
| ATOM | 1021 | O | PRO | 169 | 15.533 | −1.866 | 33.608 | 1.00 | 0.00 | 3A7 |
| ATOM | 1022 | N | VAL | 170 | 14.110 | −3.480 | 32.963 | 1.00 | 0.00 | 3A7 |
| ATOM | 1023 | CA | VAL | 170 | 15.120 | −4.462 | 32.688 | 1.00 | 0.00 | 3A7 |
| ATOM | 1024 | CB | VAL | 170 | 14.791 | −5.773 | 33.379 | 1.00 | 0.00 | 3A7 |
| ATOM | 1025 | CG1 | VAL | 170 | 15.992 | −6.742 | 33.323 | 1.00 | 0.00 | 3A7 |
| ATOM | 1026 | CG2 | VAL | 170 | 14.350 | −5.485 | 34.825 | 1.00 | 0.00 | 3A7 |
| ATOM | 1027 | C | VAL | 170 | 15.190 | −4.659 | 31.200 | 1.00 | 0.00 | 3A7 |
| ATOM | 1028 | O | VAL | 170 | 14.185 | −4.556 | 30.501 | 1.00 | 0.00 | 3A7 |
| ATOM | 1029 | N | THR | 171 | 16.400 | −4.984 | 30.690 | 1.00 | 0.00 | 3A7 |
| ATOM | 1030 | CA | THR | 171 | 16.616 | −5.364 | 29.321 | 1.00 | 0.00 | 3A7 |
| ATOM | 1031 | CB | THR | 171 | 17.889 | −4.784 | 28.730 | 1.00 | 0.00 | 3A7 |
| ATOM | 1032 | OG1 | THR | 171 | 17.858 | −3.368 | 28.857 | 1.00 | 0.00 | 3A7 |
| ATOM | 1033 | CG2 | THR | 171 | 18.018 | −5.145 | 27.233 | 1.00 | 0.00 | 3A7 |
| ATOM | 1034 | C | THR | 171 | 16.717 | −6.854 | 29.319 | 1.00 | 0.00 | 3A7 |
| ATOM | 1035 | O | THR | 171 | 17.667 | −7.434 | 29.839 | 1.00 | 0.00 | 3A7 |
| ATOM | 1036 | N | LEU | 172 | 15.716 | −7.527 | 28.719 | 1.00 | 0.00 | 3A7 |
| ATOM | 1037 | CA | LEU | 172 | 15.566 | −8.955 | 28.820 | 1.00 | 0.00 | 3A7 |
| ATOM | 1038 | CB | LEU | 172 | 14.147 | −9.409 | 28.421 | 1.00 | 0.00 | 3A7 |
| ATOM | 1039 | CG | LEU | 172 | 12.958 | −8.735 | 29.139 | 1.00 | 0.00 | 3A7 |
| ATOM | 1040 | CD1 | LEU | 172 | 13.118 | −8.664 | 30.670 | 1.00 | 0.00 | 3A7 |
| ATOM | 1041 | CD2 | LEU | 172 | 12.633 | −7.378 | 28.501 | 1.00 | 0.00 | 3A7 |
| ATOM | 1042 | C | LEU | 172 | 16.574 | −9.705 | 27.991 | 1.00 | 0.00 | 3A7 |
| ATOM | 1043 | O | LEU | 172 | 17.033 | −10.771 | 26.384 | 1.00 | 0.00 | 3A7 |
| ATOM | 1044 | N | LYS | 173 | 16.994 | −9.161 | 26.826 | 1.00 | 0.00 | 3A7 |
| ATOM | 1045 | CA | LYS | 173 | 17.961 | −9.803 | 25.952 | 1.00 | 0.00 | 3A7 |
| ATOM | 1046 | CB | LYS | 173 | 18.202 | −8.967 | 24.685 | 1.00 | 0.00 | 3A7 |
| ATOM | 1047 | CG | LYS | 173 | 19.033 | −9.687 | 23.605 | 1.00 | 0.00 | 3A7 |
| ATOM | 1048 | CD | LYS | 173 | 19.257 | −8.825 | 22.357 | 1.00 | 0.00 | 3A7 |
| ATOM | 1049 | CE | LYS | 173 | 19.931 | −9.589 | 21.211 | 1.00 | 0.00 | 3A7 |
| ATOM | 1050 | NZ | LYS | 173 | 20.065 | −8.725 | 20.017 | 1.00 | 0.00 | 3A7 |
| ATOM | 1051 | C | LYS | 173 | 19.296 | −10.057 | 26.620 | 1.00 | 0.00 | 3A7 |
| ATOM | 1052 | O | LYS | 173 | 19.912 | −11.106 | 26.445 | 1.00 | 0.00 | 3A7 |
| ATOM | 1053 | N | HIS | 174 | 19.729 | −9.103 | 27.472 | 1.00 | 0.00 | 3A7 |
| ATOM | 1054 | CA | HIS | 174 | 20.962 | −9.190 | 28.204 | 1.00 | 0.00 | 3A7 |
| ATOM | 1055 | ND1 | HIS | 174 | 23.840 | −7.893 | 28.844 | 1.00 | 0.00 | 3A7 |
| ATOM | 1056 | CG | HIS | 174 | 22.644 | −7.783 | 29.521 | 1.00 | 0.00 | 3A7 |
| ATOM | 1057 | CB | HIS | 174 | 21.288 | −7.846 | 28.865 | 1.00 | 0.00 | 3A7 |
| ATOM | 1058 | NE2 | HIS | 174 | 24.347 | −7.664 | 30.996 | 1.00 | 0.00 | 3A7 |
| ATOM | 1059 | CD2 | HIS | 174 | 22.973 | −7.645 | 30.834 | 1.00 | 0.00 | 3A7 |
| ATOM | 1060 | CE1 | HIS | 174 | 24.824 | −7.814 | 29.775 | 1.00 | 0.00 | 3A7 |
| ATOM | 1061 | C | HIS | 174 | 20.970 | −10.255 | 29.275 | 1.00 | 0.00 | 3A7 |
| ATOM | 1062 | O | HIS | 174 | 21.877 | −11.082 | 29.317 | 1.00 | 0.00 | 3A7 |
| ATOM | 1063 | N | VAL | 175 | 19.954 | −10.272 | 30.170 | 1.00 | 0.00 | 3A7 |
| ATOM | 1064 | CA | VAL | 175 | 19.876 | −11.181 | 31.303 | 1.00 | 0.00 | 3A7 |
| ATOM | 1065 | CB | VAL | 175 | 18.832 | −10.715 | 32.291 | 1.00 | 0.00 | 3A7 |
| ATOM | 1066 | CG1 | VAL | 175 | 18.836 | −11.586 | 33.568 | 1.00 | 0.00 | 3A7 |
| ATOM | 1067 | CG2 | VAL | 175 | 19.117 | −9.239 | 32.645 | 1.00 | 0.00 | 3A7 |
| ATOM | 1068 | C | VAL | 175 | 19.575 | −12.603 | 30.872 | 1.00 | 0.00 | 3A7 |
| ATOM | 1069 | O | VAL | 175 | 20.055 | −13.576 | 31.455 | 1.00 | 0.00 | 3A7 |
| ATOM | 1070 | N | PHE | 176 | 18.769 | −12.751 | 29.799 | 1.00 | 0.00 | 3A7 |
| ATOM | 1071 | CA | PHE | 176 | 18.383 | −14.047 | 29.302 | 1.00 | 0.00 | 3A7 |
| ATOM | 1072 | CB | PHE | 176 | 17.044 | −13.978 | 28.573 | 1.00 | 0.00 | 3A7 |
| ATOM | 1073 | CG | PHE | 176 | 15.984 | −14.019 | 29.651 | 1.00 | 0.00 | 3A7 |
| ATOM | 1074 | CD1 | PHE | 176 | 15.271 | −12.887 | 30.048 | 1.00 | 0.00 | 3A7 |
| ATOM | 1075 | CD2 | PHE | 176 | 15.760 | −15.216 | 30.337 | 1.00 | 0.00 | 3A7 |
| ATOM | 1076 | CE1 | PHE | 176 | 14.380 | −12.941 | 31.117 | 1.00 | 0.00 | 3A7 |
| ATOM | 1077 | CE2 | PHE | 176 | 14.857 | −15.282 | 31.394 | 1.00 | 0.00 | 3A7 |
| ATOM | 1078 | CZ | PHE | 176 | 14.171 | −14.139 | 31.790 | 1.00 | 0.00 | 3A7 |
| ATOM | 1079 | C | PHE | 176 | 19.411 | −14.648 | 28.382 | 1.00 | 0.00 | 3A7 |
| ATOM | 1080 | O | PHE | 176 | 19.429 | −15.860 | 28.180 | 1.00 | 0.00 | 3A7 |
| ATOM | 1081 | N | GLY | 177 | 20.328 | −13.824 | 27.829 | 1.00 | 0.00 | 3A7 |
| ATOM | 1082 | CA | GLY | 177 | 21.447 | −14.314 | 27.061 | 1.00 | 0.00 | 3A7 |
| ATOM | 1083 | C | GLY | 177 | 22.530 | −14.791 | 27.997 | 1.00 | 0.00 | 3A7 |
| ATOM | 1084 | O | GLY | 177 | 23.231 | −15.756 | 27.714 | 1.00 | 0.00 | 3A7 |
| ATOM | 1085 | N | ALA | 178 | 22.657 | −14.142 | 29.180 | 1.00 | 0.00 | 3A7 |
| ATOM | 1086 | CA | ALA | 178 | 23.616 | −14.483 | 30.214 | 1.00 | 0.00 | 3A7 |
| ATOM | 1087 | CB | ALA | 178 | 23.639 | −13.426 | 31.327 | 1.00 | 0.00 | 3A7 |
| ATOM | 1088 | C | ALA | 178 | 23.303 | −15.805 | 30.855 | 1.00 | 0.00 | 3A7 |
| ATOM | 1089 | O | ALA | 178 | 24.178 | −16.635 | 31.070 | 1.00 | 0.00 | 3A7 |
| ATOM | 1090 | N | TYR | 179 | 22.004 | −16.063 | 31.127 | 1.00 | 0.00 | 3A7 |
| ATOM | 1091 | CA | TYR | 179 | 21.522 | −17.288 | 31.732 | 1.00 | 0.00 | 3A7 |
| ATOM | 1092 | CB | TYR | 179 | 19.979 | −17.174 | 31.929 | 1.00 | 0.00 | 3A7 |
| ATOM | 1093 | CG | TYR | 179 | 19.104 | −18.432 | 31.952 | 1.00 | 0.00 | 3A7 |
| ATOM | 1094 | CD1 | TYR | 179 | 18.538 | −18.892 | 33.140 | 1.00 | 0.00 | 3A7 |
| ATOM | 1095 | CD2 | TYR | 179 | 18.795 | −19.133 | 30.778 | 1.00 | 0.00 | 3A7 |
| ATOM | 1096 | CE1 | TYR | 179 | 17.873 | −20.112 | 33.200 | 1.00 | 0.00 | 3A7 |
| ATOM | 1097 | CE2 | TYR | 179 | 18.128 | −20.354 | 30.824 | 1.00 | 0.00 | 3A7 |
| ATOM | 1098 | CZ | TYR | 179 | 17.709 | −20.868 | 32.046 | 1.00 | 0.00 | 3A7 |
| ATOM | 1099 | OH | TYR | 179 | 17.108 | −22.143 | 32.117 | 1.00 | 0.00 | 3A7 |
| ATOM | 1100 | C | TYR | 179 | 21.881 | −18.504 | 30.914 | 1.00 | 0.00 | 3A7 |
| ATOM | 1101 | O | TYR | 179 | 22.420 | −19.487 | 31.413 | 1.00 | 0.00 | 3A7 |
| ATOM | 1102 | N | SER | 180 | 21.607 | −18.432 | 29.602 | 1.00 | 0.00 | 3A7 |
| ATOM | 1103 | CA | SER | 180 | 21.758 | −19.553 | 28.719 | 1.00 | 0.00 | 3A7 |
| ATOM | 1104 | CB | SER | 180 | 20.943 | −19.328 | 27.464 | 1.00 | 0.00 | 3A7 |
| ATOM | 1105 | OG | SER | 180 | 21.331 | −18.109 | 26.860 | 1.00 | 0.00 | 3A7 |
| ATOM | 1106 | C | SER | 180 | 23.198 | −19.852 | 28.402 | 1.00 | 0.00 | 3A7 |
| ATOM | 1107 | O | SER | 180 | 23.565 | −21.010 | 28.278 | 1.00 | 0.00 | 3A7 |
| ATOM | 1108 | N | MET | 181 | 24.084 | −18.834 | 28.319 | 1.00 | 0.00 | 3A7 |
| ATOM | 1109 | CA | MET | 181 | 25.507 | −19.052 | 28.132 | 1.00 | 0.00 | 3A7 |
| ATOM | 1110 | CB | MET | 181 | 26.246 | −17.722 | 27.978 | 1.00 | 0.00 | 3A7 |
| ATOM | 1111 | CG | MET | 181 | 25.986 | −17.169 | 26.568 | 1.00 | 0.00 | 3A7 |
| ATOM | 1112 | SD | MET | 181 | 26.894 | −15.667 | 26.130 | 1.00 | 0.00 | 3A7 |
| ATOM | 1113 | CE | MET | 181 | 25.919 | −14.578 | 27.203 | 1.00 | 0.00 | 3A7 |
| ATOM | 1114 | C | MET | 181 | 26.127 | −19.780 | 29.282 | 1.00 | 0.00 | 3A7 |
| ATOM | 1115 | O | MET | 181 | 26.923 | −20.700 | 29.117 | 1.00 | 0.00 | 3A7 |
| ATOM | 1116 | N | ASP | 182 | 25.695 | −19.407 | 30.493 | 1.00 | 0.00 | 3A7 |
| ATOM | 1117 | CA | ASP | 182 | 26.174 | −20.008 | 31.695 | 1.00 | 0.00 | 3A7 |
| ATOM | 1118 | CB | ASP | 182 | 25.809 | −19.160 | 32.908 | 1.00 | 0.00 | 3A7 |
| ATOM | 1119 | CG | ASP | 182 | 26.545 | −17.813 | 32.877 | 1.00 | 0.00 | 3A7 |
| ATOM | 1120 | OD1 | ASP | 182 | 27.380 | −17.590 | 31.961 | 1.00 | 0.00 | 3A7 |
| ATOM | 1121 | OD2 | ASP | 182 | 26.276 | −16.986 | 33.788 | 1.00 | 0.00 | 3A7 |
| ATOM | 1122 | C | ASP | 182 | 25.645 | −21.408 | 31.886 | 1.00 | 0.00 | 3A7 |
| ATOM | 1123 | O | ASP | 182 | 26.371 | −22.258 | 32.382 | 1.00 | 0.00 | 3A7 |
| ATOM | 1124 | N | VAL | 183 | 24.401 | −21.724 | 31.448 | 1.00 | 0.00 | 3A7 |
| ATOM | 1125 | CA | VAL | 183 | 23.887 | −23.084 | 31.514 | 1.00 | 0.00 | 3A7 |
| ATOM | 1126 | CB | VAL | 183 | 22.419 | −23.173 | 31.124 | 1.00 | 0.00 | 3A7 |
| ATOM | 1127 | CG1 | VAL | 183 | 21.919 | −24.632 | 30.984 | 1.00 | 0.00 | 3A7 |
| ATOM | 1128 | CG2 | VAL | 183 | 21.578 | −22.468 | 32.200 | 1.00 | 0.00 | 3A7 |
| ATOM | 1129 | C | VAL | 183 | 24.678 | −24.016 | 30.610 | 1.00 | 0.00 | 3A7 |
| ATOM | 1130 | O | VAL | 183 | 25.065 | −25.103 | 31.027 | 1.00 | 0.00 | 3A7 |
| ATOM | 1131 | N | ILE | 184 | 24.999 | −23.597 | 29.353 | 1.00 | 0.00 | 3A7 |
| ATOM | 1132 | CA | ILE | 184 | 25.762 | −24.395 | 28.439 | 1.00 | 0.00 | 3A7 |
| ATOM | 1133 | CB | ILE | 184 | 25.800 | −23.816 | 27.039 | 1.00 | 0.00 | 3A7 |
| ATOM | 1134 | CG2 | ILE | 184 | 26.594 | −24.750 | 26.093 | 1.00 | 0.00 | 3A7 |
| ATOM | 1135 | CG1 | ILE | 184 | 24.365 | −23.617 | 26.489 | 1.00 | 0.00 | 3A7 |
| ATOM | 1136 | CD | ILE | 184 | 23.531 | −24.898 | 26.404 | 1.00 | 0.00 | 3A7 |
| ATOM | 1137 | C | ILE | 184 | 27.167 | −24.561 | 28.928 | 1.00 | 0.00 | 3A7 |
| ATOM | 1138 | O | ILE | 184 | 27.720 | −25.652 | 28.859 | 1.00 | 0.00 | 3A7 |
| ATOM | 1139 | N | THR | 185 | 27.774 | −23.489 | 29.486 | 1.00 | 0.00 | 3A7 |
| ATOM | 1140 | CA | THR | 185 | 29.125 | −23.511 | 30.002 | 1.00 | 0.00 | 3A7 |
| ATOM | 1141 | CB | THR | 185 | 29.617 | −22.111 | 30.359 | 1.00 | 0.00 | 3A7 |
| ATOM | 1142 | OG1 | THR | 185 | 29.625 | −21.307 | 29.187 | 1.00 | 0.00 | 3A7 |
| ATOM | 1143 | CG2 | THR | 185 | 31.047 | −22.108 | 30.943 | 1.00 | 0.00 | 3A7 |
| ATOM | 1144 | C | THR | 185 | 29.259 | −24.418 | 31.192 | 1.00 | 0.00 | 3A7 |
| ATOM | 1145 | O | THR | 185 | 30.220 | −25.166 | 31.343 | 1.00 | 0.00 | 3A7 |
| ATOM | 1146 | N | SER | 186 | 28.242 | −24.404 | 32.066 | 1.00 | 0.00 | 3A7 |
| ATOM | 1147 | CA | SER | 186 | 28.262 | −25.188 | 33.262 | 1.00 | 0.00 | 3A7 |
| ATOM | 1148 | CB | SER | 186 | 27.223 | −24.719 | 34.259 | 1.00 | 0.00 | 3A7 |
| ATOM | 1149 | OG | SER | 186 | 27.539 | −23.412 | 34.719 | 1.00 | 0.00 | 3A7 |
| ATOM | 1150 | C | SER | 186 | 28.037 | −26.655 | 33.000 | 1.00 | 0.00 | 3A7 |
| ATOM | 1151 | O | SER | 186 | 28.693 | −27.500 | 33.598 | 1.00 | 0.00 | 3A7 |
| ATOM | 1152 | N | THR | 187 | 27.128 | −27.006 | 32.060 | 1.00 | 0.00 | 3A7 |
| ATOM | 1153 | CA | THR | 187 | 26.865 | −28.386 | 31.711 | 1.00 | 0.00 | 3A7 |
| ATOM | 1154 | CB | THR | 187 | 25.561 | −28.580 | 30.943 | 1.00 | 0.00 | 3A7 |
| ATOM | 1155 | OG1 | THR | 187 | 25.480 | −27.748 | 29.789 | 1.00 | 0.00 | 3A7 |
| ATOM | 1156 | CG2 | THR | 187 | 24.359 | −28.285 | 31.861 | 1.00 | 0.00 | 3A7 |
| ATOM | 1157 | C | THR | 187 | 27.994 | −28.983 | 30.897 | 1.00 | 0.00 | 3A7 |
| ATOM | 1158 | O | THR | 187 | 28.202 | −30.195 | 30.899 | 1.00 | 0.00 | 3A7 |
| ATOM | 1159 | N | SER | 188 | 28.739 | −28.115 | 30.176 | 1.00 | 0.00 | 3A7 |
| ATOM | 1160 | CA | SER | 188 | 29.767 | −28.556 | 29.280 | 1.00 | 0.00 | 3A7 |
| ATOM | 1161 | CB | SER | 188 | 29.905 | −27.697 | 27.991 | 1.00 | 0.00 | 3A7 |
| ATOM | 1162 | OG | SER | 188 | 30.272 | −26.347 | 28.250 | 1.00 | 0.00 | 3A7 |
| ATOM | 1163 | C | SER | 188 | 31.105 | −28.690 | 29.970 | 1.00 | 0.00 | 3A7 |
| ATOM | 1164 | O | SER | 188 | 31.803 | −29.681 | 29.768 | 1.00 | 0.00 | 3A7 |
| ATOM | 1165 | N | PHE | 189 | 31.513 | −27.672 | 30.766 | 1.00 | 0.00 | 3A7 |
| ATOM | 1166 | CA | PHE | 189 | 32.870 | −27.581 | 31.256 | 1.00 | 0.00 | 3A7 |
| ATOM | 1167 | CB | PHE | 189 | 33.653 | −26.363 | 30.732 | 1.00 | 0.00 | 3A7 |
| ATOM | 1168 | CG | PHE | 189 | 33.446 | −26.027 | 29.300 | 1.00 | 0.00 | 3A7 |
| ATOM | 1169 | CD1 | PHE | 189 | 33.204 | −24.694 | 28.976 | 1.00 | 0.00 | 3A7 |
| ATOM | 1170 | CD2 | PHE | 189 | 33.468 | −26.986 | 28.292 | 1.00 | 0.00 | 3A7 |
| ATOM | 1171 | CE1 | PHE | 189 | 32.971 | −24.317 | 27.665 | 1.00 | 0.00 | 3A7 |
| ATOM | 1172 | CE2 | PHE | 189 | 33.182 | −26.618 | 26.984 | 1.00 | 0.00 | 3A7 |
| ATOM | 1173 | CZ | PHE | 189 | 32.933 | −25.284 | 26.668 | 1.00 | 0.00 | 3A7 |
| ATOM | 1174 | C | PHE | 189 | 32.919 | −27.463 | 32.762 | 1.00 | 0.00 | 3A7 |
| ATOM | 1175 | O | PHE | 189 | 33.993 | −27.441 | 33.361 | 1.00 | 0.00 | 3A7 |
| ATOM | 1176 | N | GLY | 190 | 31.743 | −27.378 | 33.435 | 1.00 | 0.00 | 3A7 |
| ATOM | 1177 | CA | GLY | 190 | 31.654 | −27.345 | 34.883 | 1.00 | 0.00 | 3A7 |
| ATOM | 1178 | C | GLY | 190 | 31.869 | −25.956 | 35.418 | 1.00 | 0.00 | 3A7 |
| ATOM | 1179 | O | GLY | 190 | 31.005 | −25.095 | 35.280 | 1.00 | 0.00 | 3A7 |
| ATOM | 1180 | N | VAL | 191 | 33.057 | −25.744 | 36.044 | 1.00 | 0.00 | 3A7 |
| ATOM | 1181 | CA | VAL | 191 | 33.597 | −24.510 | 36.596 | 1.00 | 0.00 | 3A7 |
| ATOM | 1182 | CB | VAL | 191 | 33.426 | −23.250 | 35.727 | 1.00 | 0.00 | 3A7 |
| ATOM | 1183 | CG1 | VAL | 191 | 32.199 | −22.372 | 36.078 | 1.00 | 0.00 | 3A7 |
| ATOM | 1184 | CG2 | VAL | 191 | 34.729 | −22.424 | 35.812 | 1.00 | 0.00 | 3A7 |
| ATOM | 1185 | C | VAL | 191 | 33.100 | −24.310 | 38.012 | 1.00 | 0.00 | 3A7 |
| ATOM | 1186 | O | VAL | 191 | 31.935 | −24.551 | 38.325 | 1.00 | 0.00 | 3A7 |
| ATOM | 1187 | N | SER | 192 | 34.012 | −23.844 | 38.900 | 1.00 | 0.00 | 3A7 |
| ATOM | 1188 | CA | SER | 192 | 33.727 | −23.531 | 40.281 | 1.00 | 0.00 | 3A7 |
| ATOM | 1189 | CB | SER | 192 | 34.779 | −24.116 | 41.252 | 1.00 | 0.00 | 3A7 |
| ATOM | 1190 | OG | SER | 192 | 34.799 | −25.534 | 41.156 | 1.00 | 0.00 | 3A7 |
| ATOM | 1191 | C | SER | 192 | 33.716 | −22.032 | 40.410 | 1.00 | 0.00 | 3A7 |
| ATOM | 1192 | O | SER | 192 | 34.136 | −21.317 | 39.501 | 1.00 | 0.00 | 3A7 |
| ATOM | 1193 | N | ILE | 193 | 33.230 | −21.524 | 41.572 | 1.00 | 0.00 | 3A7 |
| ATOM | 1194 | CA | ILE | 193 | 33.138 | −20.109 | 41.867 | 1.00 | 0.00 | 3A7 |
| ATOM | 1195 | CB | ILE | 193 | 31.890 | −19.756 | 42.671 | 1.00 | 0.00 | 3A7 |
| ATOM | 1196 | CG2 | ILE | 193 | 31.794 | −18.225 | 42.861 | 1.00 | 0.00 | 3A7 |
| ATOM | 1197 | CG1 | ILE | 193 | 30.615 | −20.322 | 41.992 | 1.00 | 0.00 | 3A7 |
| ATOM | 1198 | CD | ILE | 193 | 30.351 | −19.784 | 40.581 | 1.00 | 0.00 | 3A7 |
| ATOM | 1199 | C | ILE | 193 | 34.389 | −19.720 | 42.618 | 1.00 | 0.00 | 3A7 |
| ATOM | 1200 | O | ILE | 193 | 34.518 | −19.967 | 43.816 | 1.00 | 0.00 | 3A7 |
| ATOM | 1201 | N | ASP | 194 | 35.344 | −19.102 | 41.887 | 1.00 | 0.00 | 3A7 |
| ATOM | 1202 | CA | ASP | 194 | 36.617 | −18.685 | 42.420 | 1.00 | 0.00 | 3A7 |
| ATOM | 1203 | CB | ASP | 194 | 37.706 | −19.786 | 42.267 | 1.00 | 0.00 | 3A7 |
| ATOM | 1204 | CG | ASP | 194 | 38.984 | −19.433 | 43.034 | 1.00 | 0.00 | 3A7 |
| ATOM | 1205 | OD1 | ASP | 194 | 38.899 | −19.270 | 44.280 | 1.00 | 0.00 | 3A7 |
| ATOM | 1206 | OD2 | ASP | 194 | 40.056 | −19.326 | 42.381 | 1.00 | 0.00 | 3A7 |
| ATOM | 1207 | C | ASP | 194 | 36.993 | −17.442 | 41.654 | 1.00 | 0.00 | 3A7 |
| ATOM | 1208 | O | ASP | 194 | 37.756 | −16.608 | 42.141 | 1.00 | 0.00 | 3A7 |
| ATOM | 1209 | N | SER | 195 | 36.443 | −17.299 | 40.420 | 1.00 | 0.00 | 3A7 |
| ATOM | 1210 | CA | SER | 195 | 36.648 | −16.163 | 39.548 | 1.00 | 0.00 | 3A7 |
| ATOM | 1211 | CB | SER | 195 | 36.679 | −16.561 | 38.055 | 1.00 | 0.00 | 3A7 |
| ATOM | 1212 | OG | SER | 195 | 37.748 | −17.464 | 37.811 | 1.00 | 0.00 | 3A7 |
| ATOM | 1213 | C | SER | 195 | 35.523 | −15.185 | 39.763 | 1.00 | 0.00 | 3A7 |
| ATOM | 1214 | O | SER | 195 | 34.456 | −15.547 | 40.256 | 1.00 | 0.00 | 3A7 |
| ATOM | 1215 | N | LEU | 196 | 35.759 | −13.902 | 39.386 | 1.00 | 0.00 | 3A7 |
| ATOM | 1216 | CA | LEU | 196 | 34.826 | −12.813 | 39.580 | 1.00 | 0.00 | 3A7 |
| ATOM | 1217 | CB | LEU | 196 | 35.494 | −11.523 | 40.138 | 1.00 | 0.00 | 3A7 |
| ATOM | 1218 | CG | LEU | 196 | 36.127 | −11.639 | 41.551 | 1.00 | 0.00 | 3A7 |
| ATOM | 1219 | CD1 | LEU | 196 | 35.140 | −12.190 | 42.599 | 1.00 | 0.00 | 3A7 |
| ATOM | 1220 | CD2 | LEU | 196 | 37.469 | −12.399 | 41.574 | 1.00 | 0.00 | 3A7 |
| ATOM | 1221 | C | LEU | 196 | 34.175 | −12.481 | 38.258 | 1.00 | 0.00 | 3A7 |
| ATOM | 1222 | O | LEU | 196 | 33.796 | −11.334 | 38.022 | 1.00 | 0.00 | 3A7 |
| ATOM | 1223 | N | ASN | 197 | 34.036 | −13.503 | 37.366 | 1.00 | 0.00 | 3A7 |
| ATOM | 1224 | CA | ASN | 197 | 33.382 | −13.455 | 36.068 | 1.00 | 0.00 | 3A7 |
| ATOM | 1225 | CB | ASN | 197 | 31.946 | −12.846 | 36.138 | 1.00 | 0.00 | 3A7 |
| ATOM | 1226 | CG | ASN | 197 | 31.125 | −13.111 | 34.865 | 1.00 | 0.00 | 3A7 |
| ATOM | 1227 | OD1 | ASN | 197 | 31.170 | −14.206 | 34.295 | 1.00 | 0.00 | 3A7 |
| ATOM | 1228 | ND2 | ASN | 197 | 30.353 | −12.069 | 34.431 | 1.00 | 0.00 | 3A7 |
| ATOM | 1229 | C | ASN | 197 | 34.255 | −12.714 | 35.072 | 1.00 | 0.00 | 3A7 |
| ATOM | 1230 | O | ASN | 197 | 33.812 | −11.770 | 34.419 | 1.00 | 0.00 | 3A7 |
| ATOM | 1231 | N | ASN | 198 | 35.541 | −13.134 | 34.960 | 1.00 | 0.00 | 3A7 |
| ATOM | 1232 | CA | ASN | 198 | 36.520 | −12.476 | 34.117 | 1.00 | 0.00 | 3A7 |
| ATOM | 1233 | CB | ASN | 198 | 37.878 | −12.191 | 34.815 | 1.00 | 0.00 | 3A7 |
| ATOM | 1234 | CG | ASN | 198 | 37.631 | −11.423 | 36.121 | 1.00 | 0.00 | 3A7 |
| ATOM | 1235 | OD1 | ASN | 198 | 37.872 | −11.949 | 37.214 | 1.00 | 0.00 | 3A7 |
| ATOM | 1236 | ND2 | ASN | 198 | 37.143 | −10.153 | 35.986 | 1.00 | 0.00 | 3A7 |
| ATOM | 1237 | C | ASN | 198 | 36.725 | −13.246 | 32.825 | 1.00 | 0.00 | 3A7 |
| ATOM | 1238 | O | ASN | 198 | 36.624 | −12.598 | 31.789 | 1.00 | 0.00 | 3A7 |
| ATOM | 1239 | N | PRO | 199 | 36.993 | −14.562 | 32.735 | 1.00 | 0.00 | 3A7 |
| ATOM | 1240 | CA | PRO | 199 | 37.251 | −15.242 | 31.473 | 1.00 | 0.00 | 3A7 |
| ATOM | 1241 | CD | PRO | 199 | 37.339 | −15.415 | 33.865 | 1.00 | 0.00 | 3A7 |
| ATOM | 1242 | CB | PRO | 199 | 37.966 | −16.553 | 31.866 | 1.00 | 0.00 | 3A7 |
| ATOM | 1243 | CG | PRO | 199 | 38.421 | −16.338 | 33.313 | 1.00 | 0.00 | 3A7 |
| ATOM | 1244 | C | PRO | 199 | 35.959 | −15.538 | 30.731 | 1.00 | 0.00 | 3A7 |
| ATOM | 1245 | O | PRO | 199 | 36.016 | −15.766 | 29.524 | 1.00 | 0.00 | 3A7 |
| ATOM | 1246 | N | GLN | 200 | 34.800 | −15.560 | 31.441 | 1.00 | 0.00 | 3A7 |
| ATOM | 1247 | CA | GLN | 200 | 33.519 | −15.967 | 30.914 | 1.00 | 0.00 | 3A7 |
| ATOM | 1248 | CB | GLN | 200 | 32.614 | −16.559 | 32.023 | 1.00 | 0.00 | 3A7 |
| ATOM | 1249 | CG | GLN | 200 | 31.297 | −17.173 | 31.515 | 1.00 | 0.00 | 3A7 |
| ATOM | 1250 | CD | GLN | 200 | 30.581 | −17.885 | 32.668 | 1.00 | 0.00 | 3A7 |
| ATOM | 1251 | OE1 | GLN | 200 | 30.411 | −19.110 | 32.645 | 1.00 | 0.00 | 3A7 |
| ATOM | 1252 | NE2 | GLN | 200 | 30.150 | −17.085 | 33.690 | 1.00 | 0.00 | 3A7 |
| ATOM | 1253 | C | GLN | 200 | 32.815 | −14.819 | 30.232 | 1.00 | 0.00 | 3A7 |
| ATOM | 1254 | O | GLN | 200 | 32.140 | −15.035 | 29.233 | 1.00 | 0.00 | 3A7 |
| ATOM | 1255 | N | ASP | 201 | 32.961 | −13.568 | 30.743 | 1.00 | 0.00 | 3A7 |
| ATOM | 1256 | CA | ASP | 201 | 32.288 | −12.384 | 30.219 | 1.00 | 0.00 | 3A7 |
| ATOM | 1257 | CB | ASP | 201 | 32.223 | −11.254 | 31.288 | 1.00 | 0.00 | 3A7 |
| ATOM | 1258 | CG | ASP | 201 | 31.197 | −10.174 | 30.927 | 1.00 | 0.00 | 3A7 |
| ATOM | 1259 | OD1 | ASP | 201 | 31.610 | −8.994 | 30.772 | 1.00 | 0.00 | 3A7 |
| ATOM | 1260 | OD2 | ASP | 201 | 29.992 | −10.520 | 30.805 | 1.00 | 0.00 | 3A7 |
| ATOM | 1261 | C | ASP | 201 | 32.876 | −11.859 | 28.899 | 1.00 | 0.00 | 3A7 |
| ATOM | 1262 | O | ASP | 201 | 32.107 | −11.342 | 28.095 | 1.00 | 0.00 | 3A7 |
| ATOM | 1263 | N | PRO | 202 | 34.177 | −11.966 | 28.590 | 1.00 | 0.00 | 3A7 |
| ATOM | 1264 | CA | PRO | 202 | 34.768 | −11.731 | 27.282 | 1.00 | 0.00 | 3A7 |
| ATOM | 1265 | CD | PRO | 202 | 35.191 | −11.898 | 29.612 | 1.00 | 0.00 | 3A7 |
| ATOM | 1266 | CB | PRO | 202 | 36.280 | −11.790 | 27.543 | 1.00 | 0.00 | 3A7 |
| ATOM | 1267 | CG | PRO | 202 | 36.411 | −11.259 | 28.958 | 1.00 | 0.00 | 3A7 |
| ATOM | 1268 | C | PRO | 202 | 34.334 | −12.755 | 26.270 | 1.00 | 0.00 | 3A7 |
| ATOM | 1269 | O | PRO | 202 | 34.099 | −12.386 | 25.126 | 1.00 | 0.00 | 3A7 |
| ATOM | 1270 | N | PHE | 203 | 34.165 | −14.041 | 26.662 | 1.00 | 0.00 | 3A7 |
| ATOM | 1271 | CA | PHE | 203 | 33.692 | −15.113 | 25.807 | 1.00 | 0.00 | 3A7 |
| ATOM | 1272 | CB | PHE | 203 | 33.533 | −16.402 | 26.628 | 1.00 | 0.00 | 3A7 |
| ATOM | 1273 | CG | PHE | 203 | 33.530 | −17.655 | 25.800 | 1.00 | 0.00 | 3A7 |
| ATOM | 1274 | CD1 | PHE | 203 | 34.701 | −18.401 | 25.685 | 1.00 | 0.00 | 3A7 |
| ATOM | 1275 | CD2 | PHE | 203 | 32.364 | −18.126 | 25.192 | 1.00 | 0.00 | 3A7 |
| ATOM | 1276 | CE1 | PHE | 203 | 34.713 | −19.599 | 24.981 | 1.00 | 0.00 | 3A7 |
| ATOM | 1277 | CE2 | PHE | 203 | 32.377 | −19.316 | 24.471 | 1.00 | 0.00 | 3A7 |
| ATOM | 1278 | CZ | PHE | 203 | 33.547 | −20.060 | 24.377 | 1.00 | 0.00 | 3A7 |
| ATOM | 1279 | C | PHE | 203 | 32.360 | −14.780 | 25.173 | 1.00 | 0.00 | 3A7 |
| ATOM | 1280 | O | PHE | 203 | 32.125 | −15.040 | 23.999 | 1.00 | 0.00 | 3A7 |
| ATOM | 1281 | N | VAL | 204 | 31.455 | −14.147 | 25.959 | 1.00 | 0.00 | 3A7 |
| ATOM | 1282 | CA | VAL | 204 | 30.141 | −13.686 | 25.564 | 1.00 | 0.00 | 3A7 |
| ATOM | 1283 | CB | VAL | 204 | 29.470 | −12.914 | 26.710 | 1.00 | 0.00 | 3A7 |
| ATOM | 1284 | CG1 | VAL | 204 | 28.141 | −12.240 | 26.286 | 1.00 | 0.00 | 3A7 |
| ATOM | 1285 | CG2 | VAL | 204 | 29.308 | −13.845 | 27.927 | 1.00 | 0.00 | 3A7 |
| ATOM | 1286 | C | VAL | 204 | 30.210 | −12.702 | 24.430 | 1.00 | 0.00 | 3A7 |
| ATOM | 1287 | O | VAL | 204 | 29.521 | −12.835 | 23.424 | 1.00 | 0.00 | 3A7 |
| ATOM | 1288 | N | GLU | 205 | 31.077 | −11.674 | 24.576 | 1.00 | 0.00 | 3A7 |
| ATOM | 1289 | CA | GLU | 205 | 31.235 | −10.607 | 23.617 | 1.00 | 0.00 | 3A7 |
| ATOM | 1290 | CB | GLU | 205 | 32.215 | −9.544 | 24.146 | 1.00 | 0.00 | 3A7 |
| ATOM | 1291 | CG | GLU | 205 | 32.242 | −8.242 | 23.327 | 1.00 | 0.00 | 3A7 |
| ATOM | 1292 | CD | GLU | 205 | 33.217 | −7.267 | 23.979 | 1.00 | 0.00 | 3A7 |
| ATOM | 1293 | OE1 | GLU | 205 | 32.974 | −6.880 | 25.153 | 1.00 | 0.00 | 3A7 |
| ATOM | 1294 | OE2 | GLU | 205 | 34.219 | −6.896 | 23.310 | 1.00 | 0.00 | 3A7 |
| ATOM | 1295 | C | GLU | 205 | 31.739 | −11.106 | 22.289 | 1.00 | 0.00 | 3A7 |
| ATOM | 1296 | O | GLU | 205 | 31.308 | −10.646 | 21.238 | 1.00 | 0.00 | 3A7 |
| ATOM | 1297 | N | ASN | 206 | 32.629 | −12.123 | 22.316 | 1.00 | 0.00 | 3A7 |
| ATOM | 1298 | CA | ASN | 206 | 33.175 | −12.741 | 21.134 | 1.00 | 0.00 | 3A7 |
| ATOM | 1299 | CB | ASN | 206 | 34.358 | −13.657 | 21.467 | 1.00 | 0.00 | 3A7 |
| ATOM | 1300 | CG | ASN | 206 | 35.509 | −12.819 | 22.025 | 1.00 | 0.00 | 3A7 |
| ATOM | 1301 | OD1 | ASN | 206 | 35.798 | −12.848 | 23.224 | 1.00 | 0.00 | 3A7 |
| ATOM | 1302 | ND2 | ASN | 206 | 36.155 | −12.023 | 21.120 | 1.00 | 0.00 | 3A7 |
| ATOM | 1303 | C | ASN | 206 | 32.151 | −13.539 | 20.380 | 1.00 | 0.00 | 3A7 |
| ATOM | 1304 | O | ASN | 206 | 31.976 | −13.337 | 19.182 | 1.00 | 0.00 | 3A7 |
| ATOM | 1305 | N | THR | 207 | 31.417 | −14.452 | 21.067 | 1.00 | 0.00 | 3A7 |
| ATOM | 1306 | CA | THR | 207 | 30.406 | −15.301 | 20.464 | 1.00 | 0.00 | 3A7 |
| ATOM | 1307 | CB | THR | 207 | 29.749 | −16.188 | 21.502 | 1.00 | 0.00 | 3A7 |
| ATOM | 1308 | OG1 | THR | 207 | 30.751 | −16.917 | 22.199 | 1.00 | 0.00 | 3A7 |
| ATOM | 1309 | CG2 | THR | 207 | 28.780 | −17.201 | 20.851 | 1.00 | 0.00 | 3A7 |
| ATOM | 1310 | C | THR | 207 | 29.346 | −14.492 | 19.768 | 1.00 | 0.00 | 3A7 |
| ATOM | 1311 | O | THR | 207 | 28.951 | −14.790 | 18.646 | 1.00 | 0.00 | 3A7 |
| ATOM | 1312 | N | LYS | 208 | 28.929 | −13.379 | 20.411 | 1.00 | 0.00 | 3A7 |
| ATOM | 1313 | CA | LYS | 208 | 27.951 | −12.459 | 19.890 | 1.00 | 0.00 | 3A7 |
| ATOM | 1314 | CB | LYS | 208 | 27.604 | −11.413 | 20.954 | 1.00 | 0.00 | 3A7 |
| ATOM | 1315 | CG | LYS | 208 | 26.398 | −10.524 | 20.601 | 1.00 | 0.00 | 3A7 |
| ATOM | 1316 | CD | LYS | 208 | 25.914 | −9.608 | 21.741 | 1.00 | 0.00 | 3A7 |
| ATOM | 1317 | CE | LYS | 208 | 26.801 | −8.383 | 22.018 | 1.00 | 0.00 | 3A7 |
| ATOM | 1318 | NZ | LYS | 208 | 28.077 | −8.751 | 22.673 | 1.00 | 0.00 | 3A7 |
| ATOM | 1319 | C | LYS | 208 | 28.389 | −11.760 | 18.619 | 1.00 | 0.00 | 3A7 |
| ATOM | 1320 | O | LYS | 208 | 27.616 | −11.653 | 17.675 | 1.00 | 0.00 | 3A7 |
| ATOM | 1321 | N | LYS | 209 | 29.664 | −11.316 | 18.536 | 1.00 | 0.00 | 3A7 |
| ATOM | 1322 | CA | LYS | 209 | 30.209 | −10.686 | 17.348 | 1.00 | 0.00 | 3A7 |
| ATOM | 1323 | CB | LYS | 209 | 31.622 | −10.129 | 17.587 | 1.00 | 0.00 | 3A7 |
| ATOM | 1324 | CG | LYS | 209 | 31.660 | −8.877 | 18.478 | 1.00 | 0.00 | 3A7 |
| ATOM | 1325 | CD | LYS | 209 | 33.007 | −8.133 | 18.418 | 1.00 | 0.00 | 3A7 |
| ATOM | 1326 | CE | LYS | 209 | 34.227 | −8.963 | 18.843 | 1.00 | 0.00 | 3A7 |
| ATOM | 1327 | NZ | LYS | 209 | 34.144 | −9.343 | 20.270 | 1.00 | 0.00 | 3A7 |
| ATOM | 1328 | C | LYS | 209 | 30.275 | −11.624 | 16.153 | 1.00 | 0.00 | 3A7 |
| ATOM | 1329 | O | LYS | 209 | 29.942 | −11.252 | 15.031 | 1.00 | 0.00 | 3A7 |
| ATOM | 1330 | N | LEU | 210 | 30.662 | −12.901 | 16.376 | 1.00 | 0.00 | 3A7 |
| ATOM | 1331 | CA | LEU | 210 | 30.592 | −13.968 | 15.391 | 1.00 | 0.00 | 3A7 |
| ATOM | 1332 | CB | LEU | 210 | 30.985 | −15.328 | 16.011 | 1.00 | 0.00 | 3A7 |
| ATOM | 1333 | CG | LEU | 210 | 31.127 | −16.622 | 15.108 | 1.00 | 0.00 | 3A7 |
| ATOM | 1334 | CD1 | LEU | 210 | 31.072 | −17.872 | 16.013 | 1.00 | 0.00 | 3A7 |
| ATOM | 1335 | CD2 | LEU | 210 | 30.134 | −16.893 | 13.954 | 1.00 | 0.00 | 3A7 |
| ATOM | 1336 | C | LEU | 210 | 29.217 | −14.131 | 14.809 | 1.00 | 0.00 | 3A7 |
| ATOM | 1337 | O | LEU | 210 | 29.056 | −14.197 | 13.595 | 1.00 | 0.00 | 3A7 |
| ATOM | 1338 | N | LEU | 211 | 28.185 | −14.172 | 15.680 | 1.00 | 0.00 | 3A7 |
| ATOM | 1339 | CA | LEU | 211 | 26.814 | −14.406 | 15.302 | 1.00 | 0.00 | 3A7 |
| ATOM | 1340 | CB | LEU | 211 | 25.835 | −14.273 | 16.480 | 1.00 | 0.00 | 3A7 |
| ATOM | 1341 | CG | LEU | 211 | 26.035 | −15.159 | 17.726 | 1.00 | 0.00 | 3A7 |
| ATOM | 1342 | CD1 | LEU | 211 | 24.820 | −15.056 | 18.669 | 1.00 | 0.00 | 3A7 |
| ATOM | 1343 | CD2 | LEU | 211 | 26.398 | −16.614 | 17.396 | 1.00 | 0.00 | 3A7 |
| ATOM | 1344 | C | LEU | 211 | 26.271 | −13.442 | 14.274 | 1.00 | 0.00 | 3A7 |
| ATOM | 1345 | O | LEU | 211 | 25.479 | −13.847 | 13.418 | 1.00 | 0.00 | 3A7 |
| ATOM | 1346 | N | ARG | 212 | 26.667 | −12.140 | 14.324 | 1.00 | 0.00 | 3A7 |
| ATOM | 1347 | CA | ARG | 212 | 26.196 | −11.136 | 13.386 | 1.00 | 0.00 | 3A7 |
| ATOM | 1348 | CB | ARG | 212 | 25.985 | −9.737 | 14.028 | 1.00 | 0.00 | 3A7 |
| ATOM | 1349 | CG | ARG | 212 | 27.103 | −9.170 | 14.927 | 1.00 | 0.00 | 3A7 |
| ATOM | 1350 | CD | ARG | 212 | 28.270 | −8.508 | 14.183 | 1.00 | 0.00 | 3A7 |
| ATOM | 1351 | NE | ARG | 212 | 29.078 | −7.724 | 15.175 | 1.00 | 0.00 | 3A7 |
| ATOM | 1352 | CZ | ARG | 212 | 30.393 | −7.406 | 14.970 | 1.00 | 0.00 | 3A7 |
| ATOM | 1353 | NH1 | ARG | 212 | 31.075 | −7.901 | 13.898 | 1.00 | 0.00 | 3A7 |
| ATOM | 1354 | NH2 | ARG | 212 | 31.031 | −6.581 | 15.851 | 1.00 | 0.00 | 3A7 |
| ATOM | 1355 | C | ARG | 212 | 27.097 | −11.091 | 12.169 | 1.00 | 0.00 | 3A7 |
| ATOM | 1356 | O | ARG | 212 | 28.323 | −11.083 | 12.272 | 1.00 | 0.00 | 3A7 |
| ATOM | 1357 | N | PHE | 213 | 26.441 | −11.117 | 10.976 | 1.00 | 0.00 | 3A7 |
| ATOM | 1358 | CA | PHE | 213 | 26.943 | −11.263 | 9.611 | 1.00 | 0.00 | 3A7 |
| ATOM | 1359 | CB | PHE | 213 | 27.428 | −9.944 | 8.921 | 1.00 | 0.00 | 3A7 |
| ATOM | 1360 | CG | PHE | 213 | 28.597 | −9.221 | 9.541 | 1.00 | 0.00 | 3A7 |
| ATOM | 1361 | CD1 | PHE | 213 | 28.391 | −8.264 | 10.535 | 1.00 | 0.00 | 3A7 |
| ATOM | 1362 | CD2 | PHE | 213 | 29.890 | −9.399 | 9.039 | 1.00 | 0.00 | 3A7 |
| ATOM | 1363 | CE1 | PHE | 213 | 29.455 | −7.517 | 11.032 | 1.00 | 0.00 | 3A7 |
| ATOM | 1364 | CE2 | PHE | 213 | 30.959 | −8.666 | 9.546 | 1.00 | 0.00 | 3A7 |
| ATOM | 1365 | CZ | PHE | 213 | 30.741 | −7.723 | 10.544 | 1.00 | 0.00 | 3A7 |
| ATOM | 1366 | C | PHE | 213 | 27.926 | −12.408 | 9.426 | 1.00 | 0.00 | 3A7 |
| ATOM | 1367 | O | PHE | 213 | 29.121 | −12.215 | 9.209 | 1.00 | 0.00 | 3A7 |
| ATOM | 1368 | N | ASN | 214 | 27.395 | −13.649 | 9.491 | 1.00 | 0.00 | 3A7 |
| ATOM | 1369 | CA | ASN | 214 | 28.142 | −14.839 | 9.165 | 1.00 | 0.00 | 3A7 |
| ATOM | 1370 | CB | ASN | 214 | 29.054 | −15.372 | 10.314 | 1.00 | 0.00 | 3A7 |
| ATOM | 1371 | CG | ASN | 214 | 29.994 | −16.484 | 9.818 | 1.00 | 0.00 | 3A7 |
| ATOM | 1372 | OD1 | ASN | 214 | 30.863 | −16.242 | 8.972 | 1.00 | 0.00 | 3A7 |
| ATOM | 1373 | ND2 | ASN | 214 | 29.800 | −17.720 | 10.370 | 1.00 | 0.00 | 3A7 |
| ATOM | 1374 | C | ASN | 214 | 27.135 | −15.863 | 8.671 | 1.00 | 0.00 | 3A7 |
| ATOM | 1375 | O | ASN | 214 | 27.356 | −16.351 | 7.565 | 1.00 | 0.00 | 3A7 |
| ATOM | 1376 | N | PRO | 215 | 26.035 | −16.260 | 9.344 | 1.00 | 0.00 | 3A7 |
| ATOM | 1377 | CA | PRO | 215 | 25.150 | −17.305 | 8.858 | 1.00 | 0.00 | 3A7 |
| ATOM | 1378 | CD | PRO | 215 | 25.729 | −15.929 | 10.728 | 1.00 | 0.00 | 3A7 |
| ATOM | 1379 | CB | PRO | 215 | 24.466 | −17.865 | 10.125 | 1.00 | 0.00 | 3A7 |
| ATOM | 1380 | CG | PRO | 215 | 25.216 | −17.241 | 11.310 | 1.00 | 0.00 | 3A7 |
| ATOM | 1381 | C | PRO | 215 | 24.158 | −16.681 | 7.893 | 1.00 | 0.00 | 3A7 |
| ATOM | 1382 | O | PRO | 215 | 23.256 | −15.982 | 8.353 | 1.00 | 0.00 | 3A7 |
| ATOM | 1383 | N | LEU | 216 | 24.323 | −16.921 | 6.560 | 1.00 | 0.00 | 3A7 |
| ATOM | 1384 | CA | LEU | 216 | 23.496 | −16.424 | 5.463 | 1.00 | 0.00 | 3A7 |
| ATOM | 1385 | CB | LEU | 216 | 21.971 | −16.737 | 5.575 | 1.00 | 0.00 | 3A7 |
| ATOM | 1386 | CG | LEU | 216 | 21.559 | −18.224 | 5.393 | 1.00 | 0.00 | 3A7 |
| ATOM | 1387 | CD1 | LEU | 216 | 21.970 | −18.782 | 4.017 | 1.00 | 0.00 | 3A7 |
| ATOM | 1388 | CD2 | LEU | 216 | 22.012 | −19.148 | 6.540 | 1.00 | 0.00 | 3A7 |
| ATOM | 1389 | C | LEU | 216 | 23.663 | −14.925 | 5.304 | 1.00 | 0.00 | 3A7 |
| ATOM | 1390 | O | LEU | 216 | 22.996 | −14.143 | 5.980 | 1.00 | 0.00 | 3A7 |
| ATOM | 1391 | N | ASP | 217 | 24.571 | −14.501 | 4.392 | 1.00 | 0.00 | 3A7 |
| ATOM | 1392 | CA | ASP | 217 | 24.872 | −13.102 | 4.192 | 1.00 | 0.00 | 3A7 |
| ATOM | 1393 | CB | ASP | 217 | 26.106 | −12.608 | 5.013 | 1.00 | 0.00 | 3A7 |
| ATOM | 1394 | CG | ASP | 217 | 27.385 | −13.427 | 4.786 | 1.00 | 0.00 | 3A7 |
| ATOM | 1395 | OD1 | ASP | 217 | 28.378 | −12.833 | 4.285 | 1.00 | 0.00 | 3A7 |
| ATOM | 1396 | OD2 | ASP | 217 | 27.390 | −14.641 | 5.118 | 1.00 | 0.00 | 3A7 |
| ATOM | 1397 | C | ASP | 217 | 25.056 | −12.646 | 2.709 | 1.00 | 0.00 | 3A7 |
| ATOM | 1398 | O | ASP | 217 | 26.126 | −13.116 | 2.166 | 1.00 | 0.00 | 3A7 |
| ATOM | 1399 | N | PRO | 218 | 24.053 | −12.282 | 2.013 | 1.00 | 0.00 | 3A7 |
| ATOM | 1400 | CA | PRO | 218 | 24.193 | −11.689 | 0.691 | 1.00 | 0.00 | 3A7 |
| ATOM | 1401 | CD | PRO | 218 | 22.655 | −12.450 | 2.382 | 1.00 | 0.00 | 3A7 |
| ATOM | 1402 | CB | PRO | 218 | 22.746 | −11.477 | 0.203 | 1.00 | 0.00 | 3A7 |
| ATOM | 1403 | CG | PRO | 218 | 21.908 | −12.439 | 1.050 | 1.00 | 0.00 | 3A7 |
| ATOM | 1404 | C | PRO | 218 | 24.945 | −10.372 | 0.782 | 1.00 | 0.00 | 3A7 |
| ATOM | 1405 | O | PRO | 218 | 24.911 | −9.730 | 1.833 | 1.00 | 0.00 | 3A7 |
| ATOM | 1406 | N | PHE | 219 | 25.621 | −9.950 | −0.316 | 1.00 | 0.00 | 3A7 |
| ATOM | 1407 | CA | PHE | 219 | 26.432 | −8.745 | −0.344 | 1.00 | 0.00 | 3A7 |
| ATOM | 1408 | CB | PHE | 219 | 27.591 | −8.800 | −1.373 | 1.00 | 0.00 | 3A7 |
| ATOM | 1409 | CG | PHE | 219 | 28.495 | −9.952 | −1.047 | 1.00 | 0.00 | 3A7 |
| ATOM | 1410 | CD1 | PHE | 219 | 29.296 | −9.917 | 0.094 | 1.00 | 0.00 | 3A7 |
| ATOM | 1411 | CD2 | PHE | 219 | 28.551 | −11.070 | −1.881 | 1.00 | 0.00 | 3A7 |
| ATOM | 1412 | CE1 | PHE | 219 | 30.140 | −10.981 | 0.400 | 1.00 | 0.00 | 3A7 |
| ATOM | 1413 | CE2 | PHE | 219 | 29.393 | −12.136 | −1.578 | 1.00 | 0.00 | 3A7 |
| ATOM | 1414 | CZ | PHE | 219 | 30.189 | −12.092 | −0.437 | 1.00 | 0.00 | 3A7 |
| ATOM | 1415 | C | PHE | 219 | 25.587 | −7.501 | −0.570 | 1.00 | 0.00 | 3A7 |
| ATOM | 1416 | O | PHE | 219 | 25.626 | −6.607 | 0.273 | 1.00 | 0.00 | 3A7 |
| ATOM | 1417 | N | VAL | 220 | 24.790 | −7.366 | −1.669 | 1.00 | 0.00 | 3A7 |
| ATOM | 1418 | CA | VAL | 220 | 24.617 | −8.245 | −2.807 | 1.00 | 0.00 | 3A7 |
| ATOM | 1419 | CB | VAL | 220 | 23.148 | −8.591 | −3.051 | 1.00 | 0.00 | 3A7 |
| ATOM | 1420 | CG1 | VAL | 220 | 22.271 | −7.323 | −3.127 | 1.00 | 0.00 | 3A7 |
| ATOM | 1421 | CG2 | VAL | 220 | 22.991 | −9.517 | −4.277 | 1.00 | 0.00 | 3A7 |
| ATOM | 1422 | C | VAL | 220 | 25.287 | −7.570 | −3.983 | 1.00 | 0.00 | 3A7 |
| ATOM | 1423 | O | VAL | 220 | 25.799 | −8.235 | −4.883 | 1.00 | 0.00 | 3A7 |
| ATOM | 1424 | N | LEU | 221 | 25.336 | −6.211 | −3.963 | 1.00 | 0.00 | 3A7 |
| ATOM | 1425 | CA | LEU | 221 | 26.163 | −5.403 | −4.835 | 1.00 | 0.00 | 3A7 |
| ATOM | 1426 | CB | LEU | 221 | 25.390 | −4.284 | −5.597 | 1.00 | 0.00 | 3A7 |
| ATOM | 1427 | CG | LEU | 221 | 24.479 | −4.750 | −6.764 | 1.00 | 0.00 | 3A7 |
| ATOM | 1428 | CD1 | LEU | 221 | 25.258 | −5.539 | −7.835 | 1.00 | 0.00 | 3A7 |
| ATOM | 1429 | CD2 | LEU | 221 | 23.213 | −5.501 | −6.317 | 1.00 | 0.00 | 3A7 |
| ATOM | 1430 | C | LEU | 221 | 27.196 | −4.792 | −3.913 | 1.00 | 0.00 | 3A7 |
| ATOM | 1431 | O | LEU | 221 | 27.565 | −5.391 | −2.904 | 1.00 | 0.00 | 3A7 |
| ATOM | 1432 | N | SER | 222 | 27.682 | −3.567 | −4.239 | 1.00 | 0.00 | 3A7 |
| ATOM | 1433 | CA | SER | 222 | 28.622 | −2.816 | −3.435 | 1.00 | 0.00 | 3A7 |
| ATOM | 1434 | CB | SER | 222 | 29.722 | −2.139 | −4.284 | 1.00 | 0.00 | 3A7 |
| ATOM | 1435 | OG | SER | 222 | 30.472 | −3.121 | −4.985 | 1.00 | 0.00 | 3A7 |
| ATOM | 1436 | C | SER | 222 | 27.841 | −1.762 | −2.698 | 1.00 | 0.00 | 3A7 |
| ATOM | 1437 | O | SER | 222 | 27.764 | −0.612 | −3.127 | 1.00 | 0.00 | 3A7 |
| ATOM | 1438 | N | ILE | 223 | 27.222 | −2.164 | −1.561 | 1.00 | 0.00 | 3A7 |
| ATOM | 1439 | CA | ILE | 223 | 26.322 | −1.338 | −0.788 | 1.00 | 0.00 | 3A7 |
| ATOM | 1440 | CB | ILE | 223 | 24.971 | −2.017 | −0.555 | 1.00 | 0.00 | 3A7 |
| ATOM | 1441 | CG2 | ILE | 223 | 25.156 | −3.487 | −0.110 | 1.00 | 0.00 | 3A7 |
| ATOM | 1442 | CG1 | ILE | 223 | 24.042 | −1.193 | 0.374 | 1.00 | 0.00 | 3A7 |
| ATOM | 1443 | CD | ILE | 223 | 22.603 | −1.718 | 0.416 | 1.00 | 0.00 | 3A7 |
| ATOM | 1444 | C | ILE | 223 | 27.019 | −0.926 | 0.488 | 1.00 | 0.00 | 3A7 |
| ATOM | 1445 | O | ILE | 223 | 27.093 | 0.266 | 0.787 | 1.00 | 0.00 | 3A7 |
| ATOM | 1446 | N | LYS | 224 | 27.556 | −1.893 | 1.271 | 1.00 | 0.00 | 3A7 |
| ATOM | 1447 | CA | LYS | 224 | 28.263 | −1.581 | 2.490 | 1.00 | 0.00 | 3A7 |
| ATOM | 1448 | CB | LYS | 224 | 27.339 | −1.185 | 3.674 | 1.00 | 0.00 | 3A7 |
| ATOM | 1449 | CG | LYS | 224 | 28.088 | −0.528 | 4.849 | 1.00 | 0.00 | 3A7 |
| ATOM | 1450 | CD | LYS | 224 | 27.190 | −0.187 | 6.049 | 1.00 | 0.00 | 3A7 |
| ATOM | 1451 | CE | LYS | 224 | 26.740 | −1.411 | 6.861 | 1.00 | 0.00 | 3A7 |
| ATOM | 1452 | NZ | LYS | 224 | 27.901 | −2.112 | 7.458 | 1.00 | 0.00 | 3A7 |
| ATOM | 1453 | C | LYS | 224 | 29.052 | −2.796 | 2.890 | 1.00 | 0.00 | 3A7 |
| ATOM | 1454 | O | LYS | 224 | 30.070 | −2.683 | 3.572 | 1.00 | 0.00 | 3A7 |
| ATOM | 1455 | N | VAL | 225 | 28.582 | −4.008 | 2.483 | 1.00 | 0.00 | 3A7 |
| ATOM | 1456 | CA | VAL | 225 | 29.124 | −5.288 | 2.907 | 1.00 | 0.00 | 3A7 |
| ATOM | 1457 | CB | VAL | 225 | 28.125 | −6.430 | 2.746 | 1.00 | 0.00 | 3A7 |
| ATOM | 1458 | CG1 | VAL | 225 | 28.683 | −7.731 | 3.365 | 1.00 | 0.00 | 3A7 |
| ATOM | 1459 | CG2 | VAL | 225 | 26.805 | −6.017 | 3.429 | 1.00 | 0.00 | 3A7 |
| ATOM | 1460 | C | VAL | 225 | 30.398 | −5.583 | 2.147 | 1.00 | 0.00 | 3A7 |
| ATOM | 1461 | O | VAL | 225 | 31.486 | −5.449 | 2.708 | 1.00 | 0.00 | 3A7 |
| ATOM | 1462 | N | PHE | 226 | 30.271 | −5.986 | 0.851 | 1.00 | 0.00 | 3A7 |
| ATOM | 1463 | CA | PHE | 226 | 31.349 | −6.182 | −0.106 | 1.00 | 0.00 | 3A7 |
| ATOM | 1464 | CB | PHE | 226 | 32.367 | −4.997 | −0.098 | 1.00 | 0.00 | 3A7 |
| ATOM | 1465 | CG | PHE | 226 | 33.203 | −4.921 | −1.346 | 1.00 | 0.00 | 3A7 |
| ATOM | 1466 | CD1 | PHE | 226 | 34.585 | −5.107 | −1.283 | 1.00 | 0.00 | 3A7 |
| ATOM | 1467 | CD2 | PHE | 226 | 32.615 | −4.631 | −2.578 | 1.00 | 0.00 | 3A7 |
| ATOM | 1468 | CE1 | PHE | 226 | 35.365 | −5.025 | −2.432 | 1.00 | 0.00 | 3A7 |
| ATOM | 1469 | CE2 | PHE | 226 | 33.393 | −4.550 | −3.730 | 1.00 | 0.00 | 3A7 |
| ATOM | 1470 | CZ | PHE | 226 | 34.768 | −4.749 | −3.658 | 1.00 | 0.00 | 3A7 |
| ATOM | 1471 | C | PHE | 226 | 32.059 | −7.514 | 0.144 | 1.00 | 0.00 | 3A7 |
| ATOM | 1472 | O | PHE | 226 | 32.151 | −7.943 | 1.295 | 1.00 | 0.00 | 3A7 |
| ATOM | 1473 | N | PRO | 227 | 32.610 | −8.198 | −0.871 | 1.00 | 0.00 | 3A7 |
| ATOM | 1474 | CA | PRO | 227 | 33.431 | −9.392 | −0.698 | 1.00 | 0.00 | 3A7 |
| ATOM | 1475 | CD | PRO | 227 | 32.111 | −8.089 | −2.237 | 1.00 | 0.00 | 3A7 |
| ATOM | 1476 | CB | PRO | 227 | 33.602 | −9.944 | −2.123 | 1.00 | 0.00 | 3A7 |
| ATOM | 1477 | CG | PRO | 227 | 32.349 | −9.465 | −2.857 | 1.00 | 0.00 | 3A7 |
| ATOM | 1478 | C | PRO | 227 | 34.778 | −9.081 | −0.061 | 1.00 | 0.00 | 3A7 |
| ATOM | 1479 | O | PRO | 227 | 35.140 | −7.911 | 0.062 | 1.00 | 0.00 | 3A7 |
| ATOM | 1480 | N | PHE | 228 | 35.535 | −10.132 | 0.346 | 1.00 | 0.00 | 3A7 |
| ATOM | 1481 | CA | PHE | 228 | 36.792 | −10.004 | 1.060 | 1.00 | 0.00 | 3A7 |
| ATOM | 1482 | CB | PHE | 228 | 37.130 | −11.255 | 1.926 | 1.00 | 0.00 | 3A7 |
| ATOM | 1483 | CG | PHE | 228 | 37.053 | −12.560 | 1.172 | 1.00 | 0.00 | 3A7 |
| ATOM | 1484 | CD1 | PHE | 228 | 35.821 | −13.176 | 0.937 | 1.00 | 0.00 | 3A7 |
| ATOM | 1485 | CD2 | PHE | 228 | 38.217 | −13.193 | 0.733 | 1.00 | 0.00 | 3A7 |
| ATOM | 1486 | CE1 | PHE | 228 | 35.753 | −14.388 | 0.257 | 1.00 | 0.00 | 3A7 |
| ATOM | 1487 | CE2 | PHE | 228 | 38.152 | −14.405 | 0.053 | 1.00 | 0.00 | 3A7 |
| ATOM | 1488 | CZ | PHE | 228 | 36.919 | −15.003 | −0.187 | 1.00 | 0.00 | 3A7 |
| ATOM | 1489 | C | PHE | 228 | 37.924 | −9.676 | 0.107 | 1.00 | 0.00 | 3A7 |
| ATOM | 1490 | O | PHE | 228 | 38.323 | −10.488 | −0.727 | 1.00 | 0.00 | 3A7 |
| ATOM | 1491 | N | LEO | 229 | 38.440 | −8.432 | 0.225 | 1.00 | 0.00 | 3A7 |
| ATOM | 1492 | CA | LEO | 229 | 39.469 | −7.914 | −0.636 | 1.00 | 0.00 | 3A7 |
| ATOM | 1493 | CB | LEO | 229 | 38.924 | −7.440 | −2.010 | 1.00 | 0.00 | 3A7 |
| ATOM | 1494 | CG | LEO | 229 | 39.979 | −6.871 | −2.990 | 1.00 | 0.00 | 3A7 |
| ATOM | 1495 | CD1 | LEO | 229 | 41.083 | −7.892 | −3.326 | 1.00 | 0.00 | 3A7 |
| ATOM | 1496 | CD2 | LEO | 229 | 39.305 | −6.347 | −4.273 | 1.00 | 0.00 | 3A7 |
| ATOM | 1497 | C | LEO | 229 | 40.062 | −6.752 | 0.105 | 1.00 | 0.00 | 3A7 |
| ATOM | 1498 | O | LEO | 229 | 41.281 | −6.639 | 0.226 | 1.00 | 0.00 | 3A7 |
| ATOM | 1499 | N | THR | 230 | 39.186 | −5.847 | 0.615 | 1.00 | 0.00 | 3A7 |
| ATOM | 1500 | CA | THR | 230 | 39.568 | −4.636 | 1.308 | 1.00 | 0.00 | 3A7 |
| ATOM | 1501 | CB | THR | 230 | 38.715 | −3.444 | 0.870 | 1.00 | 0.00 | 3A7 |
| ATOM | 1502 | OG1 | THR | 230 | 39.184 | −2.215 | 1.417 | 1.00 | 0.00 | 3A7 |
| ATOM | 1503 | CG2 | THR | 230 | 37.220 | −3.639 | 1.206 | 1.00 | 0.00 | 3A7 |
| ATOM | 1504 | C | TUR | 230 | 39.476 | −4.902 | 2.807 | 1.00 | 0.00 | 3A7 |
| ATOM | 1505 | O | THR | 230 | 38.513 | −5.527 | 3.250 | 1.00 | 0.00 | 3A7 |
| ATOM | 1506 | N | PRO | 231 | 40.435 | −4.450 | 3.626 | 1.00 | 0.00 | 3A7 |
| ATOM | 1507 | CA | PRO | 231 | 40.382 | −4.534 | 5.074 | 1.00 | 0.00 | 3A7 |
| ATOM | 1508 | CD | PRO | 231 | 41.737 | −3.996 | 3.151 | 1.00 | 0.00 | 3A7 |
| ATOM | 1509 | CB | PRO | 231 | 41.866 | −4.556 | 5.472 | 1.00 | 0.00 | 3A7 |
| ATOM | 1510 | CG | PRO | 231 | 42.555 | −3.696 | 4.409 | 1.00 | 0.00 | 3A7 |
| ATOM | 1511 | C | PRO | 231 | 39.661 | −3.313 | 5.625 | 1.00 | 0.00 | 3A7 |
| ATOM | 1512 | O | PRO | 231 | 38.946 | −2.637 | 4.885 | 1.00 | 0.00 | 3A7 |
| ATOM | 1513 | N | ILE | 232 | 39.877 | −3.024 | 6.939 | 1.00 | 0.00 | 3A7 |
| ATOM | 1514 | CA | ILE | 232 | 39.462 | −1.870 | 7.738 | 1.00 | 0.00 | 3A7 |
| ATOM | 1515 | CB | ILE | 232 | 40.410 | −0.663 | 7.563 | 1.00 | 0.00 | 3A7 |
| ATOM | 1516 | CG2 | ILE | 232 | 40.450 | −0.093 | 6.123 | 1.00 | 0.00 | 3A7 |
| ATOM | 1517 | CG1 | ILE | 232 | 40.186 | 0.463 | 8.608 | 1.00 | 0.00 | 3A7 |
| ATOM | 1518 | CD | ILE | 232 | 40.563 | 0.068 | 10.038 | 1.00 | 0.00 | 3A7 |
| ATOM | 1519 | C | ILE | 232 | 37.983 | −1.493 | 7.610 | 1.00 | 0.00 | 3A7 |
| ATOM | 1520 | O | ILE | 232 | 37.631 | −0.517 | 6.949 | 1.00 | 0.00 | 3A7 |
| ATOM | 1521 | N | LEU | 233 | 37.047 | −2.244 | 8.259 | 1.00 | 0.00 | 3A7 |
| ATOM | 1522 | CA | LEU | 233 | 37.231 | −3.488 | 8.977 | 1.00 | 0.00 | 3A7 |
| ATOM | 1523 | CB | LEU | 233 | 36.638 | −3.490 | 10.418 | 1.00 | 0.00 | 3A7 |
| ATOM | 1524 | CG | LEU | 233 | 37.361 | −2.613 | 11.475 | 1.00 | 0.00 | 3A7 |
| ATOM | 1525 | CD1 | LEU | 233 | 38.832 | −3.029 | 11.668 | 1.00 | 0.00 | 3A7 |
| ATOM | 1526 | CD2 | LEU | 233 | 37.198 | −1.098 | 11.249 | 1.00 | 0.00 | 3A7 |
| ATOM | 1527 | C | LEU | 233 | 36.518 | −4.523 | 8.138 | 1.00 | 0.00 | 3A7 |
| ATOM | 1528 | O | LEU | 233 | 36.754 | −4.608 | 6.933 | 1.00 | 0.00 | 3A7 |
| ATOM | 1529 | N | GLU | 234 | 35.621 | −5.330 | 8.775 | 1.00 | 0.00 | 3A7 |
| ATOM | 1530 | CA | GLU | 234 | 34.755 | −6.337 | 8.183 | 1.00 | 0.00 | 3A7 |
| ATOM | 1531 | CB | GLU | 234 | 33.985 | −5.836 | 6.925 | 1.00 | 0.00 | 3A7 |
| ATOM | 1532 | CG | GLU | 234 | 32.750 | −6.666 | 6.514 | 1.00 | 0.00 | 3A7 |
| ATOM | 1533 | CD | GLU | 234 | 33.141 | −7.946 | 5.777 | 1.00 | 0.00 | 3A7 |
| ATOM | 1534 | OE1 | GLU | 234 | 33.797 | −7.837 | 4.707 | 1.00 | 0.00 | 3A7 |
| ATOM | 1535 | OE2 | GLU | 234 | 32.780 | −9.048 | 6.271 | 1.00 | 0.00 | 3A7 |
| ATOM | 1536 | C | GLU | 234 | 35.545 | −7.587 | 7.877 | 1.00 | 0.00 | 3A7 |
| ATOM | 1537 | O | GLU | 234 | 36.385 | −7.576 | 6.978 | 1.00 | 0.00 | 3A7 |
| ATOM | 1538 | N | ALA | 235 | 35.267 | −8.681 | 8.642 | 1.00 | 0.00 | 3A7 |
| ATOM | 1539 | CA | ALA | 235 | 35.833 | −10.014 | 8.499 | 1.00 | 0.00 | 3A7 |
| ATOM | 1540 | CB | ALA | 235 | 36.269 | −10.390 | 7.067 | 1.00 | 0.00 | 3A7 |
| ATOM | 1541 | C | ALA | 235 | 37.017 | −10.214 | 9.409 | 1.00 | 0.00 | 3A7 |
| ATOM | 1542 | O | ALA | 235 | 37.273 | −11.312 | 9.899 | 1.00 | 0.00 | 3A7 |
| ATOM | 1543 | N | LEU | 236 | 37.790 | −9.141 | 9.671 | 1.00 | 0.00 | 3A7 |
| ATOM | 1544 | CA | LEU | 236 | 38.971 | −9.206 | 10.503 | 1.00 | 0.00 | 3A7 |
| ATOM | 1545 | CB | LEU | 236 | 39.828 | −7.935 | 10.361 | 1.00 | 0.00 | 3A7 |
| ATOM | 1546 | CG | LEU | 236 | 40.242 | −7.612 | 8.906 | 1.00 | 0.00 | 3A7 |
| ATOM | 1547 | CD1 | LEU | 236 | 40.964 | −6.254 | 8.836 | 1.00 | 0.00 | 3A7 |
| ATOM | 1548 | CD2 | LEU | 236 | 41.094 | −8.728 | 8.272 | 1.00 | 0.00 | 3A7 |
| ATOM | 1549 | C | LEU | 236 | 38.601 | −9.366 | 11.956 | 1.00 | 0.00 | 3A7 |
| ATOM | 1550 | O | LEU | 236 | 39.204 | −10.138 | 12.697 | 1.00 | 0.00 | 3A7 |
| ATOM | 1551 | N | ASN | 237 | 37.530 | −8.664 | 12.387 | 1.00 | 0.00 | 3A7 |
| ATOM | 1552 | CA | ASN | 237 | 37.020 | −8.738 | 13.741 | 1.00 | 0.00 | 3A7 |
| ATOM | 1553 | CB | ASN | 237 | 35.917 | −7.685 | 13.982 | 1.00 | 0.00 | 3A7 |
| ATOM | 1554 | CG | ASN | 237 | 36.463 | −6.273 | 13.746 | 1.00 | 0.00 | 3A7 |
| ATOM | 1555 | OD1 | ASN | 237 | 35.922 | −5.526 | 12.923 | 1.00 | 0.00 | 3A7 |
| ATOM | 1556 | ND2 | ASN | 237 | 37.544 | −5.910 | 14.500 | 1.00 | 0.00 | 3A7 |
| ATOM | 1557 | C | ASN | 237 | 36.444 | −10.107 | 14.022 | 1.00 | 0.00 | 3A7 |
| ATOM | 1558 | O | ASN | 237 | 36.569 | −10.658 | 15.111 | 1.00 | 0.00 | 3A7 |
| ATOM | 1559 | N | ILE | 238 | 35.832 | −10.707 | 12.979 | 1.00 | 0.00 | 3A7 |
| ATOM | 1560 | CA | ILE | 238 | 35.229 | −12.011 | 13.021 | 1.00 | 0.00 | 3A7 |
| ATOM | 1561 | CB | ILE | 238 | 34.447 | −12.290 | 11.743 | 1.00 | 0.00 | 3A7 |
| ATOM | 1562 | CG2 | ILE | 238 | 33.714 | −13.648 | 11.845 | 1.00 | 0.00 | 3A7 |
| ATOM | 1563 | CG1 | ILE | 238 | 33.451 | −11.141 | 11.432 | 1.00 | 0.00 | 3A7 |
| ATOM | 1564 | CD | ILE | 238 | 32.367 | −10.932 | 12.495 | 1.00 | 0.00 | 3A7 |
| ATOM | 1565 | C | ILE | 238 | 36.280 | −13.064 | 13.260 | 1.00 | 0.00 | 3A7 |
| ATOM | 1566 | O | ILE | 238 | 36.094 | −13.896 | 14.106 | 1.00 | 0.00 | 3A7 |
| ATOM | 1567 | N | THR | 239 | 37.422 | −13.013 | 12.556 | 1.00 | 0.00 | 3A7 |
| ATOM | 1568 | CA | THR | 239 | 38.535 | −13.929 | 12.699 | 1.00 | 0.00 | 3A7 |
| ATOM | 1569 | CB | THR | 239 | 39.546 | −13.767 | 11.574 | 1.00 | 0.00 | 3A7 |
| ATOM | 1570 | OG1 | THR | 239 | 38.870 | −13.766 | 10.323 | 1.00 | 0.00 | 3A7 |
| ATOM | 1571 | CG2 | THR | 239 | 40.562 | −14.928 | 11.571 | 1.00 | 0.00 | 3A7 |
| ATOM | 1572 | C | THR | 239 | 39.226 | −13.773 | 14.033 | 1.00 | 0.00 | 3A7 |
| ATOM | 1573 | O | THR | 239 | 39.720 | −14.740 | 14.603 | 1.00 | 0.00 | 3A7 |
| ATOM | 1574 | N | VAL | 240 | 39.204 | −12.555 | 14.619 | 1.00 | 0.00 | 3A7 |
| ATOM | 1575 | CA | VAL | 240 | 39.759 | −12.276 | 15.932 | 1.00 | 0.00 | 3A7 |
| ATOM | 1576 | CB | VAL | 240 | 39.930 | −10.784 | 16.162 | 1.00 | 0.00 | 3A7 |
| ATOM | 1577 | CG1 | VAL | 240 | 40.276 | −10.421 | 17.627 | 1.00 | 0.00 | 3A7 |
| ATOM | 1578 | CG2 | VAL | 240 | 41.076 | −10.316 | 15.239 | 1.00 | 0.00 | 3A7 |
| ATOM | 1579 | C | VAL | 240 | 38.911 | −12.842 | 17.025 | 1.00 | 0.00 | 3A7 |
| ATOM | 1580 | O | VAL | 240 | 39.418 | −13.460 | 17.956 | 1.00 | 0.00 | 3A7 |
| ATOM | 1581 | N | PHE | 241 | 37.578 | −12.667 | 16.920 | 1.00 | 0.00 | 3A7 |
| ATOM | 1582 | CA | PHE | 241 | 36.634 | −13.266 | 17.837 | 1.00 | 0.00 | 3A7 |
| ATOM | 1583 | CB | PHE | 241 | 35.170 | −12.893 | 17.483 | 1.00 | 0.00 | 3A7 |
| ATOM | 1584 | CG | PHE | 241 | 34.222 | −14.041 | 17.098 | 1.00 | 0.00 | 3A7 |
| ATOM | 1585 | CD1 | PHE | 241 | 33.928 | −15.104 | 17.973 | 1.00 | 0.00 | 3A7 |
| ATOM | 1586 | CD2 | PHE | 241 | 34.038 | −14.250 | 15.746 | 1.00 | 0.00 | 3A7 |
| ATOM | 1587 | CE1 | PHE | 241 | 33.801 | −16.400 | 17.483 | 1.00 | 0.00 | 3A7 |
| ATOM | 1588 | CE2 | PHE | 241 | 34.006 | −15.545 | 15.244 | 1.00 | 0.00 | 3A7 |
| ATOM | 1589 | CZ | PHE | 241 | 33.918 | −16.626 | 16.119 | 1.00 | 0.00 | 3A7 |
| ATOM | 1590 | C | PHE | 241 | 36.784 | −14.763 | 17.938 | 1.00 | 0.00 | 3A7 |
| ATOM | 1591 | O | PHE | 241 | 36.807 | −15.276 | 19.050 | 1.00 | 0.00 | 3A7 |
| ATOM | 1592 | N | PRO | 242 | 36.833 | −15.511 | 16.814 | 1.00 | 0.00 | 3A7 |
| ATOM | 1593 | CA | PRO | 242 | 36.972 | −16.902 | 16.784 | 1.00 | 0.00 | 3A7 |
| ATOM | 1594 | CD | PRO | 242 | 37.457 | −15.196 | 15.686 | 1.00 | 0.00 | 3A7 |
| ATOM | 1595 | CB | PRO | 242 | 36.945 | −17.375 | 15.333 | 1.00 | 0.00 | 3A7 |
| ATOM | 1596 | CG | PRO | 242 | 37.313 | −16.221 | 14.553 | 1.00 | 0.00 | 3A7 |
| ATOM | 1597 | C | PRO | 242 | 38.274 | −17.310 | 17.435 | 1.00 | 0.00 | 3A7 |
| ATOM | 1598 | O | PRO | 242 | 38.251 | −18.268 | 18.191 | 1.00 | 0.00 | 3A7 |
| ATOM | 1599 | N | ARG | 243 | 39.409 | −16.603 | 17.262 | 1.00 | 0.00 | 3A7 |
| ATOM | 1600 | CA | ARG | 243 | 40.651 | −16.892 | 17.893 | 1.00 | 0.00 | 3A7 |
| ATOM | 1601 | CB | ARG | 243 | 41.734 | −15.942 | 17.355 | 1.00 | 0.00 | 3A7 |
| ATOM | 1602 | CG | ARG | 243 | 42.172 | −16.265 | 15.917 | 1.00 | 0.00 | 3A7 |
| ATOM | 1603 | CD | ARG | 243 | 43.102 | −15.187 | 15.348 | 1.00 | 0.00 | 3A7 |
| ATOM | 1604 | NE | ARG | 243 | 43.425 | −15.532 | 13.927 | 1.00 | 0.00 | 3A7 |
| ATOM | 1605 | CZ | ARG | 243 | 44.062 | −14.651 | 13.096 | 1.00 | 0.00 | 3A7 |
| ATOM | 1606 | NH1 | ARG | 243 | 44.310 | −15.003 | 11.800 | 1.00 | 0.00 | 3A7 |
| ATOM | 1607 | NH2 | ARG | 243 | 44.444 | −13.422 | 13.549 | 1.00 | 0.00 | 3A7 |
| ATOM | 1608 | C | ARG | 243 | 40.582 | −16.769 | 19.398 | 1.00 | 0.00 | 3A7 |
| ATOM | 1609 | O | ARG | 243 | 41.152 | −17.580 | 20.122 | 1.00 | 0.00 | 3A7 |
| ATOM | 1610 | N | LYS | 244 | 39.813 | −15.774 | 19.905 | 1.00 | 0.00 | 3A7 |
| ATOM | 1611 | CA | LYS | 244 | 39.642 | −15.565 | 21.327 | 1.00 | 0.00 | 3A7 |
| ATOM | 1612 | CB | LYS | 244 | 39.046 | −14.178 | 21.648 | 1.00 | 0.00 | 3A7 |
| ATOM | 1613 | CG | LYS | 244 | 39.241 | −13.772 | 23.119 | 1.00 | 0.00 | 3A7 |
| ATOM | 1614 | CD | LYS | 244 | 39.058 | −12.271 | 23.402 | 1.00 | 0.00 | 3A7 |
| ATOM | 1615 | CE | LYS | 244 | 40.110 | −11.365 | 22.745 | 1.00 | 0.00 | 3A7 |
| ATOM | 1616 | NZ | LYS | 244 | 41.474 | −11.713 | 23.205 | 1.00 | 0.00 | 3A7 |
| ATOM | 1617 | C | LYS | 244 | 38.844 | −16.684 | 21.965 | 1.00 | 0.00 | 3A7 |
| ATOM | 1618 | O | LYS | 244 | 39.175 | −17.150 | 23.051 | 1.00 | 0.00 | 3A7 |
| ATOM | 1619 | N | VAL | 245 | 37.806 | −17.201 | 21.259 | 1.00 | 0.00 | 3A7 |
| ATOM | 1620 | CA | VAL | 245 | 37.014 | −18.341 | 21.692 | 1.00 | 0.00 | 3A7 |
| ATOM | 1621 | CB | VAL | 245 | 35.821 | −18.595 | 20.774 | 1.00 | 0.00 | 3A7 |
| ATOM | 1622 | CG1 | VAL | 245 | 35.055 | −19.885 | 21.138 | 1.00 | 0.00 | 3A7 |
| ATOM | 1623 | CG2 | VAL | 245 | 34.893 | −17.368 | 20.861 | 1.00 | 0.00 | 3A7 |
| ATOM | 1624 | C | VAL | 245 | 37.868 | −19.581 | 21.753 | 1.00 | 0.00 | 3A7 |
| ATOM | 1625 | O | VAL | 245 | 37.867 | −20.306 | 22.748 | 1.00 | 0.00 | 3A7 |
| ATOM | 1626 | N | ILE | 246 | 38.674 | −19.822 | 20.690 | 1.00 | 0.00 | 3A7 |
| ATOM | 1627 | CA | ILE | 246 | 39.563 | −20.965 | 20.600 | 1.00 | 0.00 | 3A7 |
| ATOM | 1628 | CB | ILE | 246 | 40.252 | −21.026 | 19.232 | 1.00 | 0.00 | 3A7 |
| ATOM | 1629 | CG2 | ILE | 246 | 41.696 | −21.593 | 19.259 | 1.00 | 0.00 | 3A7 |
| ATOM | 1630 | CG1 | ILE | 246 | 39.414 | −21.855 | 18.224 | 1.00 | 0.00 | 3A7 |
| ATOM | 1631 | CD | ILE | 246 | 38.083 | −21.241 | 17.793 | 1.00 | 0.00 | 3A7 |
| ATOM | 1632 | C | ILE | 246 | 40.571 | −20.966 | 21.724 | 1.00 | 0.00 | 3A7 |
| ATOM | 1633 | O | ILE | 246 | 40.828 | −21.996 | 22.332 | 1.00 | 0.00 | 3A7 |
| ATOM | 1634 | N | SER | 247 | 41.133 | −19.787 | 22.059 | 1.00 | 0.00 | 3A7 |
| ATOM | 1635 | CA | SER | 247 | 42.119 | −19.652 | 23.098 | 1.00 | 0.00 | 3A7 |
| ATOM | 1636 | CB | SER | 247 | 42.734 | −18.250 | 23.119 | 1.00 | 0.00 | 3A7 |
| ATOM | 1637 | OG | SER | 247 | 43.425 | −18.000 | 21.903 | 1.00 | 0.00 | 3A7 |
| ATOM | 1638 | C | SER | 247 | 41.551 | −19.917 | 24.461 | 1.00 | 0.00 | 3A7 |
| ATOM | 1639 | O | SER | 247 | 42.194 | −20.579 | 25.271 | 1.00 | 0.00 | 3A7 |
| ATOM | 1640 | N | PHE | 248 | 40.320 | −19.434 | 24.749 | 1.00 | 0.00 | 3A7 |
| ATOM | 1641 | CA | PHE | 248 | 39.670 | −19.654 | 26.022 | 1.00 | 0.00 | 3A7 |
| ATOM | 1642 | CB | PHE | 248 | 38.328 | −18.900 | 26.125 | 1.00 | 0.00 | 3A7 |
| ATOM | 1643 | CG | PHE | 248 | 38.514 | −17.473 | 26.559 | 1.00 | 0.00 | 3A7 |
| ATOM | 1644 | CD1 | PHE | 248 | 37.951 | −16.425 | 25.828 | 1.00 | 0.00 | 3A7 |
| ATOM | 1645 | CD2 | PHE | 248 | 39.191 | −17.176 | 27.745 | 1.00 | 0.00 | 3A7 |
| ATOM | 1646 | CE1 | PHE | 248 | 38.058 | −15.111 | 26.274 | 1.00 | 0.00 | 3A7 |
| ATOM | 1647 | CE2 | PHE | 248 | 39.316 | −15.861 | 28.183 | 1.00 | 0.00 | 3A7 |
| ATOM | 1648 | CZ | PHE | 248 | 38.749 | −14.827 | 27.447 | 1.00 | 0.00 | 3A7 |
| ATOM | 1649 | C | PHE | 248 | 39.389 | −21.122 | 26.244 | 1.00 | 0.00 | 3A7 |
| ATOM | 1650 | O | PHE | 248 | 39.623 | −21.663 | 27.323 | 1.00 | 0.00 | 3A7 |
| ATOM | 1651 | N | LEU | 249 | 38.919 | −21.832 | 25.198 | 1.00 | 0.00 | 3A7 |
| ATOM | 1652 | CA | LEU | 249 | 38.585 | −23.234 | 25.317 | 1.00 | 0.00 | 3A7 |
| ATOM | 1653 | CB | LEU | 249 | 37.717 | −23.699 | 24.147 | 1.00 | 0.00 | 3A7 |
| ATOM | 1654 | CG | LEU | 249 | 36.376 | −22.962 | 24.103 | 1.00 | 0.00 | 3A7 |
| ATOM | 1655 | CD1 | LEU | 249 | 35.616 | −23.308 | 22.821 | 1.00 | 0.00 | 3A7 |
| ATOM | 1656 | CD2 | LEU | 249 | 35.503 | −23.243 | 25.342 | 1.00 | 0.00 | 3A7 |
| ATOM | 1657 | C | LEU | 249 | 39.811 | −24.111 | 25.396 | 1.00 | 0.00 | 3A7 |
| ATOM | 1658 | O | LEU | 249 | 39.841 | −25.088 | 26.140 | 1.00 | 0.00 | 3A7 |
| ATOM | 1659 | N | THR | 250 | 40.891 | −23.744 | 24.670 | 1.00 | 0.00 | 3A7 |
| ATOM | 1660 | CA | THR | 250 | 42.150 | −24.464 | 24.706 | 1.00 | 0.00 | 3A7 |
| ATOM | 1661 | CB | THR | 250 | 43.131 | −23.926 | 23.673 | 1.00 | 0.00 | 3A7 |
| ATOM | 1662 | OG1 | THR | 250 | 42.595 | −24.120 | 22.372 | 1.00 | 0.00 | 3A7 |
| ATOM | 1663 | CG2 | THR | 250 | 44.496 | −24.649 | 23.731 | 1.00 | 0.00 | 3A7 |
| ATOM | 1664 | C | THR | 250 | 42.765 | −24.398 | 26.085 | 1.00 | 0.00 | 3A7 |
| ATOM | 1665 | O | THR | 250 | 43.326 | −25.376 | 26.573 | 1.00 | 0.00 | 3A7 |
| ATOM | 1666 | N | LYS | 251 | 42.622 | −23.243 | 26.777 | 1.00 | 0.00 | 3A7 |
| ATOM | 1667 | CA | LYS | 251 | 43.087 | −23.060 | 28.132 | 1.00 | 0.00 | 3A7 |
| ATOM | 1668 | CB | LYS | 251 | 42.941 | −21.601 | 28.592 | 1.00 | 0.00 | 3A7 |
| ATOM | 1669 | CG | LYS | 251 | 43.599 | −21.282 | 29.945 | 1.00 | 0.00 | 3A7 |
| ATOM | 1670 | CD | LYS | 251 | 43.466 | −19.803 | 30.335 | 1.00 | 0.00 | 3A7 |
| ATOM | 1671 | CE | LYS | 251 | 44.093 | −19.494 | 31.700 | 1.00 | 0.00 | 3A7 |
| ATOM | 1672 | NZ | LYS | 251 | 43.930 | −18.062 | 32.038 | 1.00 | 0.00 | 3A7 |
| ATOM | 1673 | C | LYS | 251 | 42.337 | −23.933 | 29.091 | 1.00 | 0.00 | 3A7 |
| ATOM | 1674 | O | LYS | 251 | 42.946 | −24.559 | 29.945 | 1.00 | 0.00 | 3A7 |
| ATOM | 1675 | N | SER | 252 | 40.996 | −24.057 | 28.942 | 1.00 | 0.00 | 3A7 |
| ATOM | 1676 | CA | SER | 252 | 40.169 | −24.912 | 29.776 | 1.00 | 0.00 | 3A7 |
| ATOM | 1677 | CB | SER | 252 | 38.679 | −24.780 | 29.422 | 1.00 | 0.00 | 3A7 |
| ATOM | 1678 | OG | SER | 252 | 38.257 | −23.431 | 29.562 | 1.00 | 0.00 | 3A7 |
| ATOM | 1679 | C | SER | 252 | 40.543 | −26.371 | 29.662 | 1.00 | 0.00 | 3A7 |
| ATOM | 1680 | O | SER | 252 | 40.637 | −27.089 | 30.653 | 1.00 | 0.00 | 3A7 |
| ATOM | 1681 | N | VAL | 253 | 40.836 | −26.832 | 28.425 | 1.00 | 0.00 | 3A7 |
| ATOM | 1682 | CA | VAL | 253 | 41.211 | −28.203 | 28.125 | 1.00 | 0.00 | 3A7 |
| ATOM | 1683 | CB | VAL | 253 | 41.292 | −28.416 | 26.609 | 1.00 | 0.00 | 3A7 |
| ATOM | 1684 | CG1 | VAL | 253 | 41.865 | −29.803 | 26.237 | 1.00 | 0.00 | 3A7 |
| ATOM | 1685 | CG2 | VAL | 253 | 39.886 | −28.264 | 25.997 | 1.00 | 0.00 | 3A7 |
| ATOM | 1686 | C | VAL | 253 | 42.532 | −28.553 | 28.758 | 1.00 | 0.00 | 3A7 |
| ATOM | 1687 | O | VAL | 253 | 42.684 | −29.586 | 29.404 | 1.00 | 0.00 | 3A7 |
| ATOM | 1688 | N | LYS | 254 | 43.518 | −27.644 | 28.616 | 1.00 | 0.00 | 3A7 |
| ATOM | 1689 | CA | LYS | 254 | 44.842 | −27.794 | 29.162 | 1.00 | 0.00 | 3A7 |
| ATOM | 1690 | CB | LYS | 254 | 45.735 | −26.640 | 28.706 | 1.00 | 0.00 | 3A7 |
| ATOM | 1691 | CG | LYS | 254 | 47.220 | −26.788 | 29.086 | 1.00 | 0.00 | 3A7 |
| ATOM | 1692 | CD | LYS | 254 | 48.143 | −25.705 | 28.496 | 1.00 | 0.00 | 3A7 |
| ATOM | 1693 | CE | LYS | 254 | 48.079 | −24.338 | 29.199 | 1.00 | 0.00 | 3A7 |
| ATOM | 1694 | NZ | LYS | 254 | 46.811 | −23.622 | 28.925 | 1.00 | 0.00 | 3A7 |
| ATOM | 1695 | C | LYS | 254 | 44.830 | −27.834 | 30.665 | 1.00 | 0.00 | 3A7 |
| ATOM | 1696 | O | LYS | 254 | 45.412 | −28.728 | 31.270 | 1.00 | 0.00 | 3A7 |
| ATOM | 1697 | N | GLN | 255 | 44.098 | −26.892 | 31.313 | 1.00 | 0.00 | 3A7 |
| ATOM | 1698 | CA | GLN | 255 | 43.943 | −26.855 | 32.753 | 1.00 | 0.00 | 3A7 |
| ATOM | 1699 | CB | GLN | 255 | 43.122 | −25.635 | 33.238 | 1.00 | 0.00 | 3A7 |
| ATOM | 1700 | CG | GLN | 255 | 43.863 | −24.302 | 33.045 | 1.00 | 0.00 | 3A7 |
| ATOM | 1701 | CD | GLN | 255 | 42.977 | −23.153 | 33.535 | 1.00 | 0.00 | 3A7 |
| ATOM | 1702 | OE1 | GLN | 255 | 43.332 | −22.444 | 34.484 | 1.00 | 0.00 | 3A7 |
| ATOM | 1703 | NE2 | GLN | 255 | 41.801 | −22.974 | 32.860 | 1.00 | 0.00 | 3A7 |
| ATOM | 1704 | C | GLN | 255 | 43.283 | −28.104 | 33.282 | 1.00 | 0.00 | 3A7 |
| ATOM | 1705 | O | GLN | 255 | 43.599 | −28.568 | 34.372 | 1.00 | 0.00 | 3A7 |
| ATOM | 1706 | N | ILE | 256 | 42.375 | −28.710 | 32.490 | 1.00 | 0.00 | 3A7 |
| ATOM | 1707 | CA | ILE | 256 | 41.679 | −29.907 | 32.888 | 1.00 | 0.00 | 3A7 |
| ATOM | 1708 | CB | ILE | 256 | 40.404 | −30.098 | 32.072 | 1.00 | 0.00 | 3A7 |
| ATOM | 1709 | CG2 | ILE | 256 | 40.068 | −31.597 | 31.831 | 1.00 | 0.00 | 3A7 |
| ATOM | 1710 | CG1 | ILE | 256 | 39.198 | −29.508 | 32.862 | 1.00 | 0.00 | 3A7 |
| ATOM | 1711 | CD | ILE | 256 | 39.270 | −28.036 | 33.277 | 1.00 | 0.00 | 3A7 |
| ATOM | 1712 | C | ILE | 256 | 42.566 | −31.136 | 32.826 | 1.00 | 0.00 | 3A7 |
| ATOM | 1713 | O | ILE | 256 | 42.411 | −32.060 | 33.627 | 1.00 | 0.00 | 3A7 |
| ATOM | 1714 | N | LYS | 257 | 43.535 | −31.169 | 31.881 | 1.00 | 0.00 | 3A7 |
| ATOM | 1715 | CA | LYS | 257 | 44.464 | −32.272 | 31.759 | 1.00 | 0.00 | 3A7 |
| ATOM | 1716 | CB | LYS | 257 | 45.216 | −32.240 | 30.418 | 1.00 | 0.00 | 3A7 |
| ATOM | 1717 | CG | LYS | 257 | 44.304 | −32.548 | 29.218 | 1.00 | 0.00 | 3A7 |
| ATOM | 1718 | CD | LYS | 257 | 45.027 | −32.676 | 27.864 | 1.00 | 0.00 | 3A7 |
| ATOM | 1719 | CE | LYS | 257 | 45.583 | −31.365 | 27.283 | 1.00 | 0.00 | 3A7 |
| ATOM | 1720 | NZ | LYS | 257 | 46.799 | −30.908 | 27.996 | 1.00 | 0.00 | 3A7 |
| ATOM | 1721 | C | LYS | 257 | 45.489 | −32.284 | 32.873 | 1.00 | 0.00 | 3A7 |
| ATOM | 1722 | O | LYS | 257 | 45.944 | −33.343 | 33.303 | 1.00 | 0.00 | 3A7 |
| ATOM | 1723 | N | GLU | 258 | 45.856 | −31.083 | 33.378 | 1.00 | 0.00 | 3A7 |
| ATOM | 1724 | CA | CLU | 258 | 46.817 | −30.925 | 34.446 | 1.00 | 0.00 | 3A7 |
| ATOM | 1725 | CB | GLU | 258 | 47.454 | −29.519 | 34.428 | 1.00 | 0.00 | 3A7 |
| ATOM | 1726 | CG | GLU | 258 | 48.242 | −29.217 | 33.141 | 1.00 | 0.00 | 3A7 |
| ATOM | 1727 | CD | GLU | 258 | 49.402 | −30.199 | 33.003 | 1.00 | 0.00 | 3A7 |
| ATOM | 1728 | OE1 | GLU | 258 | 50.286 | −30.202 | 33.900 | 1.00 | 0.00 | 3A7 |
| ATOM | 1729 | OE2 | GLU | 258 | 49.418 | −30.958 | 31.997 | 1.00 | 0.00 | 3A7 |
| ATOM | 1730 | C | GLU | 258 | 46.174 | −31.151 | 35.797 | 1.00 | 0.00 | 3A7 |
| ATOM | 1731 | O | GLU | 258 | 46.851 | −31.501 | 36.763 | 1.00 | 0.00 | 3A7 |
| ATOM | 1732 | N | GLY | 259 | 44.829 | −30.971 | 35.870 | 1.00 | 0.00 | 3A7 |
| ATOM | 1733 | CA | GLY | 259 | 44.026 | −31.194 | 37.052 | 1.00 | 0.00 | 3A7 |
| ATOM | 1734 | C | GLY | 259 | 43.490 | −32.598 | 37.051 | 1.00 | 0.00 | 3A7 |
| ATOM | 1735 | O | GLY | 259 | 44.004 | −33.461 | 36.340 | 1.00 | 0.00 | 3A7 |
| ATOM | 1736 | N | ARG | 260 | 42.444 | −32.842 | 37.887 | 1.00 | 0.00 | 3A7 |
| ATOM | 1737 | CA | ARG | 260 | 41.787 | −34.118 | 38.133 | 1.00 | 0.00 | 3A7 |
| ATOM | 1738 | CB | ARG | 260 | 41.489 | −34.954 | 36.853 | 1.00 | 0.00 | 3A7 |
| ATOM | 1739 | CG | ARG | 260 | 40.639 | −36.233 | 37.027 | 1.00 | 0.00 | 3A7 |
| ATOM | 1740 | CD | ARG | 260 | 39.172 | −36.004 | 37.425 | 1.00 | 0.00 | 3A7 |
| ATOM | 1741 | NE | ARG | 260 | 39.105 | −35.667 | 38.882 | 1.00 | 0.00 | 3A7 |
| ATOM | 1742 | CZ | ARG | 260 | 37.949 | −35.260 | 39.486 | 1.00 | 0.00 | 3A7 |
| ATOM | 1743 | NH1 | ARG | 260 | 37.967 | −34.927 | 40.809 | 1.00 | 0.00 | 3A7 |
| ATOM | 1744 | NH2 | ARG | 260 | 36.784 | −35.174 | 38.781 | 1.00 | 0.00 | 3A7 |
| ATOM | 1745 | C | ARG | 260 | 42.631 | −34.898 | 39.116 | 1.00 | 0.00 | 3A7 |
| ATOM | 1746 | O | ARG | 260 | 43.344 | −35.831 | 38.749 | 1.00 | 0.00 | 3A7 |
| ATOM | 1747 | N | LEU | 261 | 42.581 | −34.475 | 40.403 | 1.00 | 0.00 | 3A7 |
| ATOM | 1748 | CA | LEU | 261 | 43.473 | −34.943 | 41.438 | 1.00 | 0.00 | 3A7 |
| ATOM | 1749 | CB | LEU | 261 | 44.463 | −33.853 | 41.945 | 1.00 | 0.00 | 3A7 |
| ATOM | 1750 | CG | LEU | 261 | 43.883 | −32.584 | 42.630 | 1.00 | 0.00 | 3A7 |
| ATOM | 1751 | CD1 | LEU | 261 | 45.023 | −31.737 | 43.230 | 1.00 | 0.00 | 3A7 |
| ATOM | 1752 | CD2 | LEU | 261 | 43.009 | −31.708 | 41.709 | 1.00 | 0.00 | 3A7 |
| ATOM | 1753 | C | LEU | 261 | 42.675 | −35.510 | 42.582 | 1.00 | 0.00 | 3A7 |
| ATOM | 1754 | O | LEU | 261 | 43.042 | −36.544 | 43.138 | 1.00 | 0.00 | 3A7 |
| ATOM | 1755 | N | LYS | 262 | 41.569 | −34.835 | 42.973 | 1.00 | 0.00 | 3A7 |
| ATOM | 1756 | CA | LYS | 262 | 40.788 | −35.239 | 44.117 | 1.00 | 0.00 | 3A7 |
| ATOM | 1757 | CB | LYS | 262 | 41.421 | −34.790 | 45.462 | 1.00 | 0.00 | 3A7 |
| ATOM | 1758 | CG | LYS | 262 | 40.810 | −35.454 | 46.708 | 1.00 | 0.00 | 3A7 |
| ATOM | 1759 | CD | LYS | 262 | 41.515 | −35.043 | 48.009 | 1.00 | 0.00 | 3A7 |
| ATOM | 1760 | CE | LYS | 262 | 41.066 | −35.853 | 49.233 | 1.00 | 0.00 | 3A7 |
| ATOM | 1761 | NZ | LYS | 262 | 39.611 | −35.711 | 49.468 | 1.00 | 0.00 | 3A7 |
| ATOM | 1762 | C | LYS | 262 | 39.442 | −34.600 | 43.941 | 1.00 | 0.00 | 3A7 |
| ATOM | 1763 | O | LYS | 262 | 39.325 | −33.548 | 43.313 | 1.00 | 0.00 | 3A7 |
| ATOM | 1764 | N | GLU | 263 | 38.381 | −35.233 | 44.510 | 1.00 | 0.00 | 3A7 |
| ATOM | 1765 | CA | GLU | 263 | 37.009 | −34.774 | 44.438 | 1.00 | 0.00 | 3A7 |
| ATOM | 1766 | CB | GLU | 263 | 36.001 | −35.949 | 44.396 | 1.00 | 0.00 | 3A7 |
| ATOM | 1767 | CG | GLU | 263 | 36.178 | −36.837 | 43.152 | 1.00 | 0.00 | 3A7 |
| ATOM | 1768 | CD | GLU | 263 | 35.147 | −37.964 | 43.149 | 1.00 | 0.00 | 3A7 |
| ATOM | 1769 | OE1 | GLU | 263 | 34.322 | −38.035 | 44.098 | 1.00 | 0.00 | 3A7 |
| ATOM | 1770 | OE2 | GLU | 263 | 35.174 | −38.773 | 42.183 | 1.00 | 0.00 | 3A7 |
| ATOM | 1771 | C | GLU | 263 | 36.698 | −33.900 | 45.630 | 1.00 | 0.00 | 3A7 |
| ATOM | 1772 | O | GLU | 263 | 35.910 | −34.265 | 46.501 | 1.00 | 0.00 | 3A7 |
| ATOM | 1773 | N | THR | 264 | 37.333 | −32.704 | 45.670 | 1.00 | 0.00 | 3A7 |
| ATOM | 1774 | CA | THR | 264 | 37.170 | −31.721 | 46.715 | 1.00 | 0.00 | 3A7 |
| ATOM | 1775 | CB | THR | 264 | 38.280 | −31.765 | 47.757 | 1.00 | 0.00 | 3A7 |
| ATOM | 1776 | OG1 | THR | 264 | 38.329 | −33.054 | 48.352 | 1.00 | 0.00 | 3A7 |
| ATOM | 1777 | CG2 | THR | 264 | 38.047 | −30.721 | 48.870 | 1.00 | 0.00 | 3A7 |
| ATOM | 1778 | C | THR | 264 | 37.164 | −30.395 | 46.003 | 1.00 | 0.00 | 3A7 |
| ATOM | 1779 | O | THR | 264 | 36.480 | −29.458 | 46.415 | 1.00 | 0.00 | 3A7 |
| ATOM | 1780 | N | GLN | 265 | 37.940 | −30.300 | 44.890 | 1.00 | 0.00 | 3A7 |
| ATOM | 1781 | CA | GLN | 265 | 38.063 | −29.123 | 44.052 | 1.00 | 0.00 | 3A7 |
| ATOM | 1782 | CB | GLN | 265 | 39.417 | −29.038 | 43.298 | 1.00 | 0.00 | 3A7 |
| ATOM | 1783 | CG | GLN | 265 | 40.645 | −28.808 | 44.201 | 1.00 | 0.00 | 3A7 |
| ATOM | 1784 | CD | GLN | 265 | 40.965 | −30.055 | 45.032 | 1.00 | 0.00 | 3A7 |
| ATOM | 1785 | OE1 | GLN | 265 | 41.070 | −31.165 | 44.497 | 1.00 | 0.00 | 3A7 |
| ATOM | 1786 | NE2 | GLN | 265 | 41.132 | −29.849 | 46.374 | 1.00 | 0.00 | 3A7 |
| ATOM | 1787 | C | GLN | 265 | 36.953 | −29.140 | 43.030 | 1.00 | 0.00 | 3A7 |
| ATOM | 1788 | O | GLN | 265 | 36.287 | −28.132 | 42.802 | 1.00 | 0.00 | 3A7 |
| ATOM | 1789 | N | LYS | 266 | 36.738 | −30.320 | 42.404 | 1.00 | 0.00 | 3A7 |
| ATOM | 1790 | CA | LYS | 266 | 35.642 | −30.556 | 41.500 | 1.00 | 0.00 | 3A7 |
| ATOM | 1791 | CB | LYS | 266 | 36.035 | −30.452 | 40.002 | 1.00 | 0.00 | 3A7 |
| ATOM | 1792 | CG | LYS | 266 | 37.305 | −31.216 | 39.591 | 1.00 | 0.00 | 3A7 |
| ATOM | 1793 | CD | LYS | 266 | 37.703 | −30.959 | 38.129 | 1.00 | 0.00 | 3A7 |
| ATOM | 1794 | CE | LYS | 266 | 38.982 | −31.700 | 37.724 | 1.00 | 0.00 | 3A7 |
| ATOM | 1795 | NZ | LYS | 266 | 39.346 | −31.401 | 36.320 | 1.00 | 0.00 | 3A7 |
| ATOM | 1796 | C | LYS | 266 | 35.129 | −31.922 | 41.858 | 1.00 | 0.00 | 3A7 |
| ATOM | 1797 | O | LYS | 266 | 35.796 | −32.934 | 41.655 | 1.00 | 0.00 | 3A7 |
| ATOM | 1798 | N | HIS | 267 | 33.902 | −31.966 | 42.437 | 1.00 | 0.00 | 3A7 |
| ATOM | 1799 | CA | HIS | 267 | 33.272 | −33.168 | 42.940 | 1.00 | 0.00 | 3A7 |
| ATOM | 1800 | ND1 | HIS | 267 | 32.504 | −35.052 | 45.499 | 1.00 | 0.00 | 3A7 |
| ATOM | 1801 | CG | HIS | 267 | 31.780 | −34.098 | 44.815 | 1.00 | 0.00 | 3A7 |
| ATOM | 1802 | CB | HIS | 267 | 32.380 | −32.874 | 44.174 | 1.00 | 0.00 | 3A7 |
| ATOM | 1803 | NE2 | HIS | 267 | 30.385 | −35.724 | 45.524 | 1.00 | 0.00 | 3A7 |
| ATOM | 1804 | CD2 | HIS | 267 | 30.489 | −34.526 | 44.840 | 1.00 | 0.00 | 3A7 |
| ATOM | 1805 | CE1 | HIS | 267 | 31.620 | −35.999 | 45.901 | 1.00 | 0.00 | 3A7 |
| ATOM | 1806 | C | HIS | 267 | 32.435 | −33.802 | 41.858 | 1.00 | 0.00 | 3A7 |
| ATOM | 1807 | O | HIS | 267 | 32.255 | −35.018 | 41.834 | 1.00 | 0.00 | 3A7 |
| ATOM | 1808 | N | ARG | 268 | 31.909 | −32.963 | 40.929 | 1.00 | 0.00 | 3A7 |
| ATOM | 1809 | CA | ARG | 268 | 31.110 | −33.390 | 39.804 | 1.00 | 0.00 | 3A7 |
| ATOM | 1810 | CB | ARG | 268 | 29.939 | −32.419 | 39.501 | 1.00 | 0.00 | 3A7 |
| ATOM | 1811 | CG | ARG | 268 | 30.357 | −30.974 | 39.172 | 1.00 | 0.00 | 3A7 |
| ATOM | 1812 | CD | ARG | 268 | 29.154 | −30.049 | 38.948 | 1.00 | 0.00 | 3A7 |
| ATOM | 1813 | NE | ARG | 268 | 29.664 | −28.690 | 38.573 | 1.00 | 0.00 | 3A7 |
| ATOM | 1814 | CZ | ARG | 268 | 28.818 | −27.637 | 38.353 | 1.00 | 0.00 | 3A7 |
| ATOM | 1815 | NH1 | ARG | 268 | 29.328 | −26.423 | 37.993 | 1.00 | 0.00 | 3A7 |
| ATOM | 1816 | NH2 | ARG | 268 | 27.470 | −27.791 | 38.491 | 1.00 | 0.00 | 3A7 |
| ATOM | 1817 | C | ARG | 268 | 31.997 | −33.501 | 38.593 | 1.00 | 0.00 | 3A7 |
| ATOM | 1818 | O | ARG | 268 | 33.012 | −32.813 | 38.485 | 1.00 | 0.00 | 3A7 |
| ATOM | 1819 | N | VAL | 269 | 31.604 | −34.383 | 37.643 | 1.00 | 0.00 | 3A7 |
| ATOM | 1820 | CA | VAL | 269 | 32.279 | −34.566 | 36.378 | 1.00 | 0.00 | 3A7 |
| ATOM | 1821 | CB | VAL | 269 | 32.595 | −36.021 | 36.063 | 1.00 | 0.00 | 3A7 |
| ATOM | 1822 | CG1 | VAL | 269 | 33.733 | −36.473 | 37.001 | 1.00 | 0.00 | 3A7 |
| ATOM | 1823 | CG2 | VAL | 269 | 31.340 | −36.903 | 36.220 | 1.00 | 0.00 | 3A7 |
| ATOM | 1824 | C | VAL | 269 | 31.408 | −33.947 | 35.315 | 1.00 | 0.00 | 3A7 |
| ATOM | 1825 | O | VAL | 269 | 30.217 | −33.712 | 35.519 | 1.00 | 0.00 | 3A7 |
| ATOM | 1826 | N | ASP | 270 | 32.030 | −33.642 | 34.156 | 1.00 | 0.00 | 3A7 |
| ATOM | 1827 | CA | ASP | 270 | 31.450 | −32.836 | 33.096 | 1.00 | 0.00 | 3A7 |
| ATOM | 1828 | CB | ASP | 270 | 31.980 | −31.399 | 33.132 | 1.00 | 0.00 | 3A7 |
| ATOM | 1829 | CG | ASP | 270 | 33.510 | −31.368 | 33.034 | 1.00 | 0.00 | 3A7 |
| ATOM | 1830 | OD1 | ASP | 270 | 34.013 | −30.868 | 31.995 | 1.00 | 0.00 | 3A7 |
| ATOM | 1831 | OD2 | ASP | 270 | 34.191 | −31.809 | 33.998 | 1.00 | 0.00 | 3A7 |
| ATOM | 1832 | C | ASP | 270 | 31.710 | −33.510 | 31.781 | 1.00 | 0.00 | 3A7 |
| ATOM | 1833 | O | ASP | 270 | 32.369 | −34.547 | 31.709 | 1.00 | 0.00 | 3A7 |
| ATOM | 1834 | N | PHE | 271 | 31.187 | −32.915 | 30.684 | 1.00 | 0.00 | 3A7 |
| ATOM | 1835 | CA | PHE | 271 | 31.302 | −33.474 | 29.357 | 1.00 | 0.00 | 3A7 |
| ATOM | 1836 | CB | PHE | 271 | 30.407 | −32.722 | 28.356 | 1.00 | 0.00 | 3A7 |
| ATOM | 1837 | CG | PHE | 271 | 30.344 | −33.517 | 27.080 | 1.00 | 0.00 | 3A7 |
| ATOM | 1838 | CD1 | PHE | 271 | 30.012 | −34.875 | 27.101 | 1.00 | 0.00 | 3A7 |
| ATOM | 1839 | CD2 | PHE | 271 | 30.758 | −32.938 | 25.887 | 1.00 | 0.00 | 3A7 |
| ATOM | 1840 | CE1 | PHE | 271 | 30.211 | −35.660 | 25.972 | 1.00 | 0.00 | 3A7 |
| ATOM | 1841 | CE2 | PHE | 271 | 30.870 | −33.707 | 24.737 | 1.00 | 0.00 | 3A7 |
| ATOM | 1842 | CZ | PHE | 271 | 30.646 | −35.078 | 24.793 | 1.00 | 0.00 | 3A7 |
| ATOM | 1843 | C | PHE | 271 | 32.719 | −33.478 | 28.848 | 1.00 | 0.00 | 3A7 |
| ATOM | 1844 | O | PHE | 271 | 33.150 | −34.431 | 28.204 | 1.00 | 0.00 | 3A7 |
| ATOM | 1845 | N | LEU | 272 | 33.494 | −32.417 | 29.146 | 1.00 | 0.00 | 3A7 |
| ATOM | 1846 | CA | LEU | 272 | 34.866 | −32.326 | 28.716 | 1.00 | 0.00 | 3A7 |
| ATOM | 1847 | CB | LEU | 272 | 35.455 | −30.979 | 29.100 | 1.00 | 0.00 | 3A7 |
| ATOM | 1848 | CG | LEU | 272 | 36.816 | −30.698 | 28.482 | 1.00 | 0.00 | 3A7 |
| ATOM | 1849 | CD1 | LEU | 272 | 36.864 | −29.210 | 28.078 | 1.00 | 0.00 | 3A7 |
| ATOM | 1850 | CD2 | LEU | 272 | 38.027 | −31.011 | 29.363 | 1.00 | 0.00 | 3A7 |
| ATOM | 1851 | C | LEU | 272 | 35.735 | −33.394 | 29.311 | 1.00 | 0.00 | 3A7 |
| ATOM | 1852 | O | LEU | 272 | 36.573 | −33.979 | 28.634 | 1.00 | 0.00 | 3A7 |
| ATOM | 1853 | N | GLN | 273 | 35.522 | −33.712 | 30.606 | 1.00 | 0.00 | 3A7 |
| ATOM | 1854 | CA | GLN | 273 | 36.255 | −34.750 | 31.288 | 1.00 | 0.00 | 3A7 |
| ATOM | 1855 | CB | GLN | 273 | 35.890 | −34.805 | 32.788 | 1.00 | 0.00 | 3A7 |
| ATOM | 1856 | CG | GLN | 273 | 36.853 | −35.634 | 33.658 | 1.00 | 0.00 | 3A7 |
| ATOM | 1857 | CD | GLN | 273 | 38.222 | −34.946 | 33.686 | 1.00 | 0.00 | 3A7 |
| ATOM | 1858 | OE1 | GLN | 273 | 38.361 | −33.845 | 34.234 | 1.00 | 0.00 | 3A7 |
| ATOM | 1859 | NE2 | GLN | 273 | 39.247 | −35.617 | 33.079 | 1.00 | 0.00 | 3A7 |
| ATOM | 1860 | C | GLN | 273 | 35.971 | −36.088 | 30.668 | 1.00 | 0.00 | 3A7 |
| ATOM | 1861 | O | GLN | 273 | 36.882 | −36.879 | 30.456 | 1.00 | 0.00 | 3A7 |
| ATOM | 1862 | N | LEU | 274 | 34.685 | −36.340 | 30.310 | 1.00 | 0.00 | 3A7 |
| ATOM | 1863 | CA | LEU | 274 | 34.245 | −37.560 | 29.669 | 1.00 | 0.00 | 3A7 |
| ATOM | 1864 | CB | LEU | 274 | 32.718 | −37.631 | 29.471 | 1.00 | 0.00 | 3A7 |
| ATOM | 1865 | CG | LEU | 274 | 31.891 | −37.798 | 30.761 | 1.00 | 0.00 | 3A7 |
| ATOM | 1866 | CD1 | LEU | 274 | 30.398 | −37.811 | 30.394 | 1.00 | 0.00 | 3A7 |
| ATOM | 1867 | CD2 | LEU | 274 | 32.273 | −39.060 | 31.558 | 1.00 | 0.00 | 3A7 |
| ATOM | 1868 | C | LEU | 274 | 34.854 | −37.729 | 28.302 | 1.00 | 0.00 | 3A7 |
| ATOM | 1869 | O | LEU | 274 | 35.232 | −38.832 | 27.930 | 1.00 | 0.00 | 3A7 |
| ATOM | 1870 | N | MET | 275 | 35.003 | −36.636 | 27.521 | 1.00 | 0.00 | 3A7 |
| ATOM | 1871 | CA | MET | 275 | 35.607 | −36.702 | 26.211 | 1.00 | 0.00 | 3A7 |
| ATOM | 1872 | CB | MET | 275 | 35.400 | −35.428 | 25.399 | 1.00 | 0.00 | 3A7 |
| ATOM | 1873 | CG | MET | 275 | 33.958 | −35.154 | 24.962 | 1.00 | 0.00 | 3A7 |
| ATOM | 1874 | SD | MET | 275 | 33.857 | −33.829 | 23.713 | 1.00 | 0.00 | 3A7 |
| ATOM | 1875 | CE | MET | 275 | 34.315 | −32.435 | 24.786 | 1.00 | 0.00 | 3A7 |
| ATOM | 1876 | C | MET | 275 | 37.090 | −36.951 | 26.286 | 1.00 | 0.00 | 3A7 |
| ATOM | 1877 | O | MET | 275 | 37.642 | −37.666 | 25.458 | 1.00 | 0.00 | 3A7 |
| ATOM | 1878 | N | ILE | 276 | 37.778 | −36.381 | 27.305 | 1.00 | 0.00 | 3A7 |
| ATOM | 1879 | CA | ILE | 276 | 39.215 | −36.516 | 27.488 | 1.00 | 0.00 | 3A7 |
| ATOM | 1880 | CB | ILE | 276 | 39.723 | −35.567 | 28.575 | 1.00 | 0.00 | 3A7 |
| ATOM | 1881 | CG2 | ILE | 276 | 41.140 | −35.906 | 29.105 | 1.00 | 0.00 | 3A7 |
| ATOM | 1882 | CG1 | ILE | 276 | 39.717 | −34.100 | 28.081 | 1.00 | 0.00 | 3A7 |
| ATOM | 1883 | CD | ILE | 276 | 40.767 | −33.779 | 27.011 | 1.00 | 0.00 | 3A7 |
| ATOM | 1884 | C | ILE | 276 | 39.615 | −37.926 | 27.833 | 1.00 | 0.00 | 3A7 |
| ATOM | 1885 | O | ILE | 276 | 40.701 | −38.375 | 27.476 | 1.00 | 0.00 | 3A7 |
| ATOM | 1886 | N | ASP | 277 | 38.749 | −38.659 | 28.548 | 1.00 | 0.00 | 3A7 |
| ATOM | 1887 | CA | ASP | 277 | 39.133 | −39.958 | 29.016 | 1.00 | 0.00 | 3A7 |
| ATOM | 1888 | CB | ASP | 277 | 38.914 | −40.156 | 30.516 | 1.00 | 0.00 | 3A7 |
| ATOM | 1889 | CG | ASP | 277 | 39.827 | −39.210 | 31.302 | 1.00 | 0.00 | 3A7 |
| ATOM | 1890 | OD1 | ASP | 277 | 40.999 | −39.021 | 30.882 | 1.00 | 0.00 | 3A7 |
| ATOM | 1891 | OD2 | ASP | 277 | 39.351 | −38.657 | 32.329 | 1.00 | 0.00 | 3A7 |
| ATOM | 1892 | C | ASP | 277 | 38.346 | −41.070 | 28.476 | 1.00 | 0.00 | 3A7 |
| ATOM | 1893 | O | ASP | 277 | 38.933 | −42.095 | 28.164 | 1.00 | 0.00 | 3A7 |
| ATOM | 1894 | N | SER | 278 | 36.987 | −41.031 | 28.602 | 1.00 | 0.00 | 3A7 |
| ATOM | 1895 | CA | SER | 278 | 36.186 | −42.176 | 29.071 | 1.00 | 0.00 | 3A7 |
| ATOM | 1896 | CB | SER | 278 | 34.677 | −41.818 | 29.196 | 1.00 | 0.00 | 3A7 |
| ATOM | 1897 | OG | SER | 278 | 34.091 | −41.424 | 27.961 | 1.00 | 0.00 | 3A7 |
| ATOM | 1898 | C | SER | 278 | 36.329 | −43.499 | 28.318 | 1.00 | 0.00 | 3A7 |
| ATOM | 1899 | O | SER | 278 | 35.567 | −43.792 | 27.399 | 1.00 | 0.00 | 3A7 |
| ATOM | 1900 | N | GLN | 279 | 37.341 | −44.307 | 28.750 | 1.00 | 0.00 | 3A7 |
| ATOM | 1901 | CA | GLN | 279 | 37.851 | −45.539 | 28.167 | 1.00 | 0.00 | 3A7 |
| ATOM | 1902 | CB | GLN | 279 | 36.793 | −46.503 | 27.561 | 1.00 | 0.00 | 3A7 |
| ATOM | 1903 | CG | GLN | 279 | 35.768 | −47.000 | 28.593 | 1.00 | 0.00 | 3A7 |
| ATOM | 1904 | CD | GLN | 279 | 34.775 | −47.928 | 27.887 | 1.00 | 0.00 | 3A7 |
| ATOM | 1905 | OE1 | GLN | 279 | 35.143 | −49.016 | 27.428 | 1.00 | 0.00 | 3A7 |
| ATOM | 1906 | NE2 | GLN | 279 | 33.487 | −47.472 | 27.805 | 1.00 | 0.00 | 3A7 |
| ATOM | 1907 | C | GLN | 279 | 38.889 | −45.204 | 27.116 | 1.00 | 0.00 | 3A7 |
| ATOM | 1908 | O | GLN | 279 | 38.687 | −45.481 | 25.937 | 1.00 | 0.00 | 3A7 |
| ATOM | 1909 | N | ASN | 280 | 40.027 | −44.587 | 27.552 | 1.00 | 0.00 | 3A7 |
| ATOM | 1910 | CA | ASN | 280 | 41.202 | −44.166 | 26.787 | 1.00 | 0.00 | 3A7 |
| ATOM | 1911 | CB | ASN | 280 | 42.008 | −45.358 | 26.209 | 1.00 | 0.00 | 3A7 |
| ATOM | 1912 | CG | ASN | 280 | 42.430 | −46.296 | 27.347 | 1.00 | 0.00 | 3A7 |
| ATOM | 1913 | OD1 | ASN | 280 | 41.937 | −47.427 | 27.447 | 1.00 | 0.00 | 3A7 |
| ATOM | 1914 | ND2 | ASN | 280 | 43.366 | −45.803 | 28.213 | 1.00 | 0.00 | 3A7 |
| ATOM | 1915 | C | ASN | 280 | 40.890 | −43.183 | 25.664 | 1.00 | 0.00 | 3A7 |
| ATOM | 1916 | O | ASN | 280 | 40.934 | −43.532 | 24.487 | 1.00 | 0.00 | 3A7 |
| ATOM | 1917 | N | SER | 281 | 40.551 | −41.923 | 26.053 | 1.00 | 0.00 | 3A7 |
| ATOM | 1918 | CA | SER | 281 | 40.076 | −40.803 | 25.252 | 1.00 | 0.00 | 3A7 |
| ATOM | 1919 | CB | SER | 281 | 40.924 | −40.513 | 23.986 | 1.00 | 0.00 | 3A7 |
| ATOM | 1920 | OG | SER | 281 | 42.283 | −40.298 | 24.340 | 1.00 | 0.00 | 3A7 |
| ATOM | 1921 | C | SER | 281 | 38.621 | −40.951 | 24.837 | 1.00 | 0.00 | 3A7 |
| ATOM | 1922 | O | SER | 281 | 38.131 | −40.172 | 24.021 | 1.00 | 0.00 | 3A7 |
| ATOM | 1923 | N | LYS | 282 | 37.924 | −42.020 | 25.342 | 1.00 | 0.00 | 3A7 |
| ATOM | 1924 | CA | LYS | 282 | 36.818 | −42.723 | 24.707 | 1.00 | 0.00 | 3A7 |
| ATOM | 1925 | CB | LYS | 282 | 35.459 | −42.013 | 24.620 | 1.00 | 0.00 | 3A7 |
| ATOM | 1926 | CG | LYS | 282 | 34.288 | −42.957 | 24.294 | 1.00 | 0.00 | 3A7 |
| ATOM | 1927 | CD | LYS | 282 | 32.914 | −42.336 | 24.590 | 1.00 | 0.00 | 3A7 |
| ATOM | 1928 | CE | LYS | 282 | 31.744 | −43.301 | 24.357 | 1.00 | 0.00 | 3A7 |
| ATOM | 1929 | NZ | LYS | 282 | 31.691 | −43.746 | 22.946 | 1.00 | 0.00 | 3A7 |
| ATOM | 1930 | C | LYS | 282 | 37.194 | −43.157 | 23.334 | 1.00 | 0.00 | 3A7 |
| ATOM | 1931 | O | LYS | 282 | 37.082 | −42.407 | 22.364 | 1.00 | 0.00 | 3A7 |
| ATOM | 1932 | N | ASP | 283 | 37.706 | −44.392 | 23.270 | 1.00 | 0.00 | 3A7 |
| ATOM | 1933 | CA | ASP | 283 | 38.125 | −44.964 | 22.041 | 1.00 | 0.00 | 3A7 |
| ATOM | 1934 | CB | ASP | 283 | 39.663 | −45.088 | 21.918 | 1.00 | 0.00 | 3A7 |
| ATOM | 1935 | CG | ASP | 283 | 40.442 | −46.002 | 22.880 | 1.00 | 0.00 | 3A7 |
| ATOM | 1936 | OD1 | ASP | 283 | 41.683 | −46.097 | 22.672 | 1.00 | 0.00 | 3A7 |
| ATOM | 1937 | OD2 | ASP | 283 | 39.853 | −46.604 | 23.810 | 1.00 | 0.00 | 3A7 |
| ATOM | 1938 | C | ASP | 283 | 37.352 | −46.233 | 21.948 | 1.00 | 0.00 | 3A7 |
| ATOM | 1939 | O | ASP | 283 | 37.675 | −47.232 | 22.581 | 1.00 | 0.00 | 3A7 |
| ATOM | 1940 | N | SER | 284 | 36.258 | −46.198 | 21.157 | 1.00 | 0.00 | 3A7 |
| ATOM | 1941 | CA | SER | 284 | 35.513 | −47.378 | 20.782 | 1.00 | 0.00 | 3A7 |
| ATOM | 1942 | CB | SER | 284 | 34.115 | −47.009 | 20.210 | 1.00 | 0.00 | 3A7 |
| ATOM | 1943 | OG | SER | 284 | 34.183 | −45.995 | 19.212 | 1.00 | 0.00 | 3A7 |
| ATOM | 1944 | C | SER | 284 | 36.266 | −48.277 | 19.834 | 1.00 | 0.00 | 3A7 |
| ATOM | 1945 | O | SER | 284 | 36.990 | −49.180 | 20.242 | 1.00 | 0.00 | 3A7 |
| ATOM | 1946 | N | GLU | 285 | 36.113 | −48.092 | 18.521 | 1.00 | 0.00 | 3A7 |
| ATOM | 1947 | CA | GLU | 285 | 36.639 | −49.081 | 17.634 | 1.00 | 0.00 | 3A7 |
| ATOM | 1948 | CB | GLU | 285 | 35.795 | −49.213 | 16.369 | 1.00 | 0.00 | 3A7 |
| ATOM | 1949 | CG | GLU | 285 | 35.628 | −47.895 | 15.591 | 1.00 | 0.00 | 3A7 |
| ATOM | 1950 | CD | GLU | 285 | 34.752 | −48.143 | 14.367 | 1.00 | 0.00 | 3A7 |
| ATOM | 1951 | OE1 | GLU | 285 | 33.566 | −48.527 | 14.555 | 1.00 | 0.00 | 3A7 |
| ATOM | 1952 | OE2 | GLU | 285 | 35.255 | −47.948 | 13.228 | 1.00 | 0.00 | 3A7 |
| ATOM | 1953 | C | GLU | 285 | 38.083 | −48.834 | 17.309 | 1.00 | 0.00 | 3A7 |
| ATOM | 1954 | O | GLU | 285 | 38.621 | −49.477 | 16.447 | 1.00 | 0.00 | 3A7 |
| ATOM | 1955 | N | THR | 286 | 38.744 | −47.901 | 18.009 | 1.00 | 0.00 | 3A7 |
| ATOM | 1956 | CA | THR | 286 | 40.099 | −47.398 | 17.894 | 1.00 | 0.00 | 3A7 |
| ATOM | 1957 | CB | THR | 286 | 41.251 | −48.366 | 17.728 | 1.00 | 0.00 | 3A7 |
| ATOM | 1958 | OG1 | THR | 286 | 41.071 | −49.460 | 18.620 | 1.00 | 0.00 | 3A7 |
| ATOM | 1959 | CG2 | THR | 286 | 42.623 | −47.726 | 18.038 | 1.00 | 0.00 | 3A7 |
| ATOM | 1960 | C | THR | 286 | 40.161 | −46.319 | 16.868 | 1.00 | 0.00 | 3A7 |
| ATOM | 1961 | O | THR | 286 | 41.095 | −45.529 | 16.847 | 1.00 | 0.00 | 3A7 |
| ATOM | 1962 | N | HIS | 287 | 39.201 | −46.294 | 15.904 | 1.00 | 0.00 | 3A7 |
| ATOM | 1963 | CA | HIS | 287 | 39.294 | −45.529 | 14.676 | 1.00 | 0.00 | 3A7 |
| ATOM | 1964 | ND1 | HIS | 287 | 37.806 | −44.452 | 11.739 | 1.00 | 0.00 | 3A7 |
| ATOM | 1965 | CG | HIS | 287 | 38.588 | −45.495 | 12.192 | 1.00 | 0.00 | 3A7 |
| ATOM | 1966 | CB | HIS | 287 | 38.440 | −46.152 | 13.541 | 1.00 | 0.00 | 3A7 |
| ATOM | 1967 | NE2 | HIS | 287 | 39.260 | −44.903 | 10.120 | 1.00 | 0.00 | 3A7 |
| ATOM | 1968 | CD2 | HIS | 287 | 39.470 | −45.756 | 11.189 | 1.00 | 0.00 | 3A7 |
| ATOM | 1969 | CE1 | HIS | 287 | 38.251 | −44.140 | 10.497 | 1.00 | 0.00 | 3A7 |
| ATOM | 1970 | C | HIS | 287 | 38.868 | −44.107 | 14.911 | 1.00 | 0.00 | 3A7 |
| ATOM | 1971 | O | HIS | 287 | 37.679 | −43.797 | 14.904 | 1.00 | 0.00 | 3A7 |
| ATOM | 1972 | N | LYS | 288 | 39.880 | −43.225 | 15.122 | 1.00 | 0.00 | 3A7 |
| ATOM | 1973 | CA | LYS | 288 | 39.776 | −41.800 | 15.358 | 1.00 | 0.00 | 3A7 |
| ATOM | 1974 | CB | LYS | 288 | 38.889 | −41.036 | 14.340 | 1.00 | 0.00 | 3A7 |
| ATOM | 1975 | CG | LYS | 288 | 39.415 | −41.157 | 12.901 | 1.00 | 0.00 | 3A7 |
| ATOM | 1976 | CD | LYS | 288 | 38.528 | −40.437 | 11.877 | 1.00 | 0.00 | 3A7 |
| ATOM | 1977 | CE | LYS | 288 | 39.036 | −40.557 | 10.434 | 1.00 | 0.00 | 3A7 |
| ATOM | 1978 | NZ | LYS | 288 | 40.358 | −39.909 | 10.282 | 1.00 | 0.00 | 3A7 |
| ATOM | 1979 | C | LYS | 288 | 39.309 | −41.522 | 16.767 | 1.00 | 0.00 | 3A7 |
| ATOM | 1980 | O | LYS | 288 | 38.116 | −41.380 | 17.030 | 1.00 | 0.00 | 3A7 |
| ATOM | 1981 | N | ALA | 289 | 40.290 | −41.406 | 17.701 | 1.00 | 0.00 | 3A7 |
| ATOM | 1982 | CA | ALA | 289 | 40.092 | −40.977 | 19.070 | 1.00 | 0.00 | 3A7 |
| ATOM | 1983 | CB | ALA | 289 | 41.158 | −41.545 | 20.027 | 1.00 | 0.00 | 3A7 |
| ATOM | 1984 | C | ALA | 289 | 40.164 | −39.473 | 19.078 | 1.00 | 0.00 | 3A7 |
| ATOM | 1985 | O | ALA | 289 | 40.670 | −38.875 | 18.128 | 1.00 | 0.00 | 3A7 |
| ATOM | 1986 | N | LEU | 290 | 39.624 | −38.812 | 20.137 | 1.00 | 0.00 | 3A7 |
| ATOM | 1987 | CA | LEU | 290 | 39.349 | −37.401 | 20.081 | 1.00 | 0.00 | 3A7 |
| ATOM | 1988 | CB | LEU | 290 | 38.229 | −37.004 | 21.014 | 1.00 | 0.00 | 3A7 |
| ATOM | 1989 | CG | LEU | 290 | 36.888 | −37.703 | 20.701 | 1.00 | 0.00 | 3A7 |
| ATOM | 1990 | CD1 | LEU | 290 | 35.892 | −37.512 | 21.861 | 1.00 | 0.00 | 3A7 |
| ATOM | 1991 | CD2 | LEU | 290 | 36.312 | −37.221 | 19.355 | 1.00 | 0.00 | 3A7 |
| ATOM | 1992 | C | LEU | 290 | 40.550 | −36.573 | 20.350 | 1.00 | 0.00 | 3A7 |
| ATOM | 1993 | O | LEU | 290 | 41.191 | −36.642 | 21.390 | 1.00 | 0.00 | 3A7 |
| ATOM | 1994 | N | SER | 291 | 40.854 | −35.731 | 19.352 | 1.00 | 0.00 | 3A7 |
| ATOM | 1995 | CA | SER | 291 | 41.954 | −34.822 | 19.363 | 1.00 | 0.00 | 3A7 |
| ATOM | 1996 | CB | SER | 291 | 42.455 | −34.592 | 17.928 | 1.00 | 0.00 | 3A7 |
| ATOM | 1997 | OG | SER | 291 | 41.422 | −34.204 | 17.030 | 1.00 | 0.00 | 3A7 |
| ATOM | 1998 | C | SER | 291 | 41.471 | −33.540 | 19.957 | 1.00 | 0.00 | 3A7 |
| ATOM | 1999 | O | SER | 291 | 40.275 | −33.273 | 19.991 | 1.00 | 0.00 | 3A7 |
| ATOM | 2000 | N | ASP | 292 | 42.400 | −32.681 | 20.410 | 1.00 | 0.00 | 3A7 |
| ATOM | 2001 | CA | ASP | 292 | 42.071 | −31.438 | 21.064 | 1.00 | 0.00 | 3A7 |
| ATOM | 2002 | CB | ASP | 292 | 43.338 | −30.745 | 21.556 | 1.00 | 0.00 | 3A7 |
| ATOM | 2003 | CG | ASP | 292 | 44.042 | −31.647 | 22.570 | 1.00 | 0.00 | 3A7 |
| ATOM | 2004 | OD1 | ASP | 292 | 43.426 | −31.941 | 23.629 | 1.00 | 0.00 | 3A7 |
| ATOM | 2005 | OD2 | ASP | 292 | 45.204 | −32.050 | 22.298 | 1.00 | 0.00 | 3A7 |
| ATOM | 2006 | C | ASP | 292 | 41.334 | −30.471 | 20.170 | 1.00 | 0.00 | 3A7 |
| ATOM | 2007 | O | ASP | 292 | 40.568 | −29.641 | 20.643 | 1.00 | 0.00 | 3A7 |
| ATOM | 2008 | N | LEU | 293 | 41.506 | −30.616 | 18.834 | 1.00 | 0.00 | 3A7 |
| ATOM | 2009 | CA | LEU | 293 | 40.836 | −29.795 | 17.858 | 1.00 | 0.00 | 3A7 |
| ATOM | 2010 | CB | LEU | 293 | 41.527 | −29.876 | 16.490 | 1.00 | 0.00 | 3A7 |
| ATOM | 2011 | CG | LEU | 293 | 43.011 | −29.436 | 16.506 | 1.00 | 0.00 | 3A7 |
| ATOM | 2012 | CD1 | LEU | 293 | 43.663 | −29.663 | 15.129 | 1.00 | 0.00 | 3A7 |
| ATOM | 2013 | CD2 | LEU | 293 | 43.189 | −27.976 | 16.965 | 1.00 | 0.00 | 3A7 |
| ATOM | 2014 | C | LEU | 293 | 39.382 | −30.187 | 17.711 | 1.00 | 0.00 | 3A7 |
| ATOM | 2015 | O | LEU | 293 | 38.493 | −29.340 | 17.738 | 1.00 | 0.00 | 3A7 |
| ATOM | 2016 | N | GLU | 294 | 39.081 | −31.502 | 17.619 | 1.00 | 0.00 | 3A7 |
| ATOM | 2017 | CA | GLU | 294 | 37.722 | −31.998 | 17.504 | 1.00 | 0.00 | 3A7 |
| ATOM | 2018 | CB | GLU | 294 | 37.697 | −33.523 | 17.310 | 1.00 | 0.00 | 3A7 |
| ATOM | 2019 | CG | GLU | 294 | 38.247 | −33.957 | 15.942 | 1.00 | 0.00 | 3A7 |
| ATOM | 2020 | CD | GLU | 294 | 38.198 | −35.480 | 15.850 | 1.00 | 0.00 | 3A7 |
| ATOM | 2021 | OE1 | GLU | 294 | 37.070 | −36.040 | 15.905 | 1.00 | 0.00 | 3A7 |
| ATOM | 2022 | OE2 | GLU | 294 | 39.285 | −36.104 | 15.722 | 1.00 | 0.00 | 3A7 |
| ATOM | 2023 | C | GLU | 294 | 36.901 | −31.676 | 18.723 | 1.00 | 0.00 | 3A7 |
| ATOM | 2024 | O | GLU | 294 | 35.776 | −31.192 | 18.632 | 1.00 | 0.00 | 3A7 |
| ATOM | 2025 | N | LEU | 295 | 37.506 | −31.876 | 19.909 | 1.00 | 0.00 | 3A7 |
| ATOM | 2026 | CA | LEU | 295 | 36.915 | −31.573 | 21.190 | 1.00 | 0.00 | 3A7 |
| ATOM | 2027 | CB | LEU | 295 | 37.911 | −31.878 | 22.313 | 1.00 | 0.00 | 3A7 |
| ATOM | 2028 | CG | LEU | 295 | 38.311 | −33.358 | 22.435 | 1.00 | 0.00 | 3A7 |
| ATOM | 2029 | CD1 | LEU | 295 | 39.434 | −33.555 | 23.470 | 1.00 | 0.00 | 3A7 |
| ATOM | 2030 | CD2 | LEU | 295 | 37.094 | −34.206 | 22.783 | 1.00 | 0.00 | 3A7 |
| ATOM | 2031 | C | LEU | 295 | 36.535 | −30.119 | 21.312 | 1.00 | 0.00 | 3A7 |
| ATOM | 2032 | O | LEU | 295 | 35.404 | −29.765 | 21.643 | 1.00 | 0.00 | 3A7 |
| ATOM | 2033 | N | MET | 296 | 37.488 | −29.212 | 21.017 | 1.00 | 0.00 | 3A7 |
| ATOM | 2034 | CA | MET | 296 | 37.285 | −27.783 | 21.050 | 1.00 | 0.00 | 3A7 |
| ATOM | 2035 | CB | MET | 296 | 38.580 | −27.070 | 20.647 | 1.00 | 0.00 | 3A7 |
| ATOM | 2036 | CG | MET | 296 | 38.420 | −25.578 | 20.350 | 1.00 | 0.00 | 3A7 |
| ATOM | 2037 | SD | MET | 296 | 40.016 | −24.717 | 20.404 | 1.00 | 0.00 | 3A7 |
| ATOM | 2038 | CE | MET | 296 | 40.777 | −25.540 | 18.974 | 1.00 | 0.00 | 3A7 |
| ATOM | 2039 | C | MET | 296 | 36.195 | −27.341 | 20.113 | 1.00 | 0.00 | 3A7 |
| ATOM | 2040 | O | MET | 296 | 35.328 | −26.566 | 20.489 | 1.00 | 0.00 | 3A7 |
| ATOM | 2041 | N | ALA | 297 | 36.174 | −27.882 | 18.877 | 1.00 | 0.00 | 3A7 |
| ATOM | 2042 | CA | ALA | 297 | 35.172 | −27.555 | 17.895 | 1.00 | 0.00 | 3A7 |
| ATOM | 2043 | CB | ALA | 297 | 35.493 | −28.182 | 16.525 | 1.00 | 0.00 | 3A7 |
| ATOM | 2044 | C | ALA | 297 | 33.783 | −27.978 | 18.300 | 1.00 | 0.00 | 3A7 |
| ATOM | 2045 | O | ALA | 297 | 32.817 | −27.301 | 17.986 | 1.00 | 0.00 | 3A7 |
| ATOM | 2046 | N | GLN | 298 | 33.648 | −29.071 | 19.085 | 1.00 | 0.00 | 3A7 |
| ATOM | 2047 | CA | GLN | 298 | 32.385 | −29.501 | 19.640 | 1.00 | 0.00 | 3A7 |
| ATOM | 2048 | CB | GLN | 298 | 32.482 | −30.905 | 20.248 | 1.00 | 0.00 | 3A7 |
| ATOM | 2049 | CG | GLN | 298 | 32.651 | −32.025 | 19.207 | 1.00 | 0.00 | 3A7 |
| ATOM | 2050 | CD | GLN | 298 | 32.958 | −33.345 | 19.922 | 1.00 | 0.00 | 3A7 |
| ATOM | 2051 | OE1 | GLN | 298 | 34.080 | −33.554 | 20.400 | 1.00 | 0.00 | 3A7 |
| ATOM | 2052 | NE2 | GLN | 298 | 31.939 | −34.253 | 19.977 | 1.00 | 0.00 | 3A7 |
| ATOM | 2053 | C | GLN | 298 | 31.922 | −28.563 | 20.719 | 1.00 | 0.00 | 3A7 |
| ATOM | 2054 | O | GLN | 298 | 30.773 | −28.142 | 20.728 | 1.00 | 0.00 | 3A7 |
| ATOM | 2055 | N | SER | 299 | 32.831 | −28.160 | 21.630 | 1.00 | 0.00 | 3A7 |
| ATOM | 2056 | CA | SER | 299 | 32.535 | −27.222 | 22.690 | 1.00 | 0.00 | 3A7 |
| ATOM | 2057 | CB | SER | 299 | 33.759 | −27.023 | 23.597 | 1.00 | 0.00 | 3A7 |
| ATOM | 2058 | OG | SER | 299 | 34.175 | −28.269 | 24.141 | 1.00 | 0.00 | 3A7 |
| ATOM | 2059 | C | SER | 299 | 32.097 | −25.874 | 22.158 | 1.00 | 0.00 | 3A7 |
| ATOM | 2060 | O | SER | 299 | 31.185 | −25.252 | 22.698 | 1.00 | 0.00 | 3A7 |
| ATOM | 2061 | N | ILE | 300 | 32.719 | −25.399 | 21.047 | 1.00 | 0.00 | 3A7 |
| ATOM | 2062 | CA | ILE | 300 | 32.361 | −24.170 | 20.361 | 1.00 | 0.00 | 3A7 |
| ATOM | 2063 | CB | ILE | 300 | 33.321 | −23.832 | 19.229 | 1.00 | 0.00 | 3A7 |
| ATOM | 2064 | CG2 | ILE | 300 | 32.843 | −22.603 | 18.412 | 1.00 | 0.00 | 3A7 |
| ATOM | 2065 | CG1 | ILE | 300 | 34.732 | −23.560 | 19.774 | 1.00 | 0.00 | 3A7 |
| ATOM | 2066 | CD | ILE | 300 | 35.811 | −23.539 | 18.691 | 1.00 | 0.00 | 3A7 |
| ATOM | 2067 | C | ILE | 300 | 30.987 | −24.293 | 19.764 | 1.00 | 0.00 | 3A7 |
| ATOM | 2068 | O | ILE | 300 | 30.162 | −23.398 | 19.914 | 1.00 | 0.00 | 3A7 |
| ATOM | 2069 | N | ILE | 301 | 30.695 | −25.431 | 19.092 | 1.00 | 0.00 | 3A7 |
| ATOM | 2070 | CA | ILE | 301 | 29.418 | −25.676 | 18.455 | 1.00 | 0.00 | 3A7 |
| ATOM | 2071 | CB | ILE | 301 | 29.475 | −26.900 | 17.553 | 1.00 | 0.00 | 3A7 |
| ATOM | 2072 | CG2 | ILE | 301 | 28.192 | −27.772 | 17.525 | 1.00 | 0.00 | 3A7 |
| ATOM | 2073 | CG1 | ILE | 301 | 29.774 | −26.460 | 16.091 | 1.00 | 0.00 | 3A7 |
| ATOM | 2074 | CD | ILE | 301 | 31.053 | −25.647 | 15.869 | 1.00 | 0.00 | 3A7 |
| ATOM | 2075 | C | ILE | 301 | 28.303 | −25.792 | 19.465 | 1.00 | 0.00 | 3A7 |
| ATOM | 2076 | O | ILE | 301 | 27.182 | −25.379 | 19.205 | 1.00 | 0.00 | 3A7 |
| ATOM | 2077 | N | PHE | 302 | 28.584 | −26.318 | 20.673 | 1.00 | 0.00 | 3A7 |
| ATOM | 2078 | CA | PHE | 302 | 27.593 | −26.507 | 21.709 | 1.00 | 0.00 | 3A7 |
| ATOM | 2079 | CB | PHE | 302 | 28.212 | −27.306 | 22.878 | 1.00 | 0.00 | 3A7 |
| ATOM | 2080 | CG | PHE | 302 | 28.239 | −28.790 | 22.660 | 1.00 | 0.00 | 3A7 |
| ATOM | 2081 | CD1 | PHE | 302 | 28.348 | −29.335 | 21.379 | 1.00 | 0.00 | 3A7 |
| ATOM | 2082 | CD2 | PHE | 302 | 28.620 | −29.577 | 23.757 | 1.00 | 0.00 | 3A7 |
| ATOM | 2083 | CE1 | PHE | 302 | 28.914 | −30.584 | 21.188 | 1.00 | 0.00 | 3A7 |
| ATOM | 2084 | CE2 | PHE | 302 | 29.170 | −30.829 | 23.567 | 1.00 | 0.00 | 3A7 |
| ATOM | 2085 | CZ | PHE | 302 | 29.356 | −31.311 | 22.282 | 1.00 | 0.00 | 3A7 |
| ATOM | 2086 | C | PHE | 302 | 27.161 | −25.150 | 22.252 | 1.00 | 0.00 | 3A7 |
| ATOM | 2087 | O | PHE | 302 | 25.980 | −24.892 | 22.478 | 1.00 | 0.00 | 3A7 |
| ATOM | 2088 | N | ILE | 303 | 28.134 | −24.221 | 22.452 | 1.00 | 0.00 | 3A7 |
| ATOM | 2089 | CA | ILE | 303 | 27.866 | −22.869 | 22.966 | 1.00 | 0.00 | 3A7 |
| ATOM | 2090 | CB | ILE | 303 | 29.091 | −22.188 | 23.539 | 1.00 | 0.00 | 3A7 |
| ATOM | 2091 | CG2 | ILE | 303 | 28.716 | −20.768 | 24.038 | 1.00 | 0.00 | 3A7 |
| ATOM | 2092 | CG1 | ILE | 303 | 29.642 | −23.043 | 24.700 | 1.00 | 0.00 | 3A7 |
| ATOM | 2093 | CD | ILE | 303 | 30.952 | −22.513 | 25.280 | 1.00 | 0.00 | 3A7 |
| ATOM | 2094 | C | ILE | 303 | 27.215 | −22.030 | 21.914 | 1.00 | 0.00 | 3A7 |
| ATOM | 2095 | O | ILE | 303 | 26.217 | −21.376 | 22.173 | 1.00 | 0.00 | 3A7 |
| ATOM | 2096 | N | PHE | 304 | 27.728 | −22.042 | 20.671 | 1.00 | 0.00 | 3A7 |
| ATOM | 2097 | CA | PHE | 304 | 27.197 | −21.301 | 19.548 | 1.00 | 0.00 | 3A7 |
| ATOM | 2098 | CB | PHE | 304 | 28.117 | −21.579 | 18.314 | 1.00 | 0.00 | 3A7 |
| ATOM | 2099 | CG | PHE | 304 | 27.669 | −20.996 | 16.996 | 1.00 | 0.00 | 3A7 |
| ATOM | 2100 | CD1 | PHE | 304 | 27.839 | −19.644 | 16.704 | 1.00 | 0.00 | 3A7 |
| ATOM | 2101 | CD2 | PHE | 304 | 27.091 | −21.829 | 16.036 | 1.00 | 0.00 | 3A7 |
| ATOM | 2102 | CE1 | PHE | 304 | 27.436 | −19.134 | 15.472 | 1.00 | 0.00 | 3A7 |
| ATOM | 2103 | CE2 | PHE | 304 | 26.678 | −21.320 | 14.811 | 1.00 | 0.00 | 3A7 |
| ATOM | 2104 | CZ | PHE | 304 | 26.851 | −19.970 | 14.526 | 1.00 | 0.00 | 3A7 |
| ATOM | 2105 | C | PHE | 304 | 25.772 | −21.649 | 19.207 | 1.00 | 0.00 | 3A7 |
| ATOM | 2106 | O | PHE | 304 | 24.909 | −20.780 | 19.129 | 1.00 | 0.00 | 3A7 |
| ATOM | 2107 | N | ALA | 305 | 25.493 | −22.954 | 19.027 | 1.00 | 0.00 | 3A7 |
| ATOM | 2108 | CA | ALA | 305 | 24.183 | −23.428 | 18.672 | 1.00 | 0.00 | 3A7 |
| ATOM | 2109 | CB | ALA | 305 | 24.206 | −24.883 | 18.216 | 1.00 | 0.00 | 3A7 |
| ATOM | 2110 | C | ALA | 305 | 23.224 | −23.354 | 19.820 | 1.00 | 0.00 | 3A7 |
| ATOM | 2111 | O | ALA | 305 | 22.041 | −23.132 | 19.620 | 1.00 | 0.00 | 3A7 |
| ATOM | 2112 | N | GLY | 306 | 23.734 | −23.533 | 21.051 | 1.00 | 0.00 | 3A7 |
| ATOM | 2113 | CA | GLY | 306 | 22.903 | −23.642 | 22.219 | 1.00 | 0.00 | 3A7 |
| ATOM | 2114 | C | GLY | 306 | 22.541 | −22.346 | 22.888 | 1.00 | 0.00 | 3A7 |
| ATOM | 2115 | O | GLY | 306 | 21.518 | −22.258 | 23.561 | 1.00 | 0.00 | 3A7 |
| ATOM | 2116 | N | TYR | 307 | 23.359 | −21.285 | 22.742 | 1.00 | 0.00 | 3A7 |
| ATOM | 2117 | CA | TYR | 307 | 23.134 | −20.054 | 23.474 | 1.00 | 0.00 | 3A7 |
| ATOM | 2118 | CB | TYR | 307 | 24.473 | −19.279 | 23.690 | 1.00 | 0.00 | 3A7 |
| ATOM | 2119 | CG | TYR | 307 | 24.492 | −17.764 | 23.545 | 1.00 | 0.00 | 3A7 |
| ATOM | 2120 | CD1 | TYR | 307 | 23.675 | −16.927 | 24.308 | 1.00 | 0.00 | 3A7 |
| ATOM | 2121 | CD2 | TYR | 307 | 25.364 | −17.182 | 22.621 | 1.00 | 0.00 | 3A7 |
| ATOM | 2122 | CE1 | TYR | 307 | 23.720 | −15.545 | 24.144 | 1.00 | 0.00 | 3A7 |
| ATOM | 2123 | CE2 | TYR | 307 | 25.420 | −15.801 | 22.460 | 1.00 | 0.00 | 3A7 |
| ATOM | 2124 | CZ | TYR | 307 | 24.595 | −14.980 | 23.222 | 1.00 | 0.00 | 3A7 |
| ATOM | 2125 | OH | TYR | 307 | 24.653 | −13.578 | 23.066 | 1.00 | 0.00 | 3A7 |
| ATOM | 2126 | C | TYR | 307 | 22.056 | −19.224 | 22.841 | 1.00 | 0.00 | 3A7 |
| ATOM | 2127 | O | TYR | 307 | 21.092 | −18.851 | 23.498 | 1.00 | 0.00 | 3A7 |
| ATOM | 2128 | N | GLU | 308 | 22.222 | −18.888 | 21.552 | 1.00 | 0.00 | 3A7 |
| ATOM | 2129 | CA | GLU | 308 | 21.442 | −17.887 | 20.875 | 1.00 | 0.00 | 3A7 |
| ATOM | 2130 | CB | GLU | 308 | 22.048 | −17.593 | 19.488 | 1.00 | 0.00 | 3A7 |
| ATOM | 2131 | CG | GLU | 308 | 22.280 | −18.862 | 18.631 | 1.00 | 0.00 | 3A7 |
| ATOM | 2132 | CD | GLU | 308 | 23.057 | −18.569 | 17.347 | 1.00 | 0.00 | 3A7 |
| ATOM | 2133 | OE1 | GLU | 308 | 23.277 | −17.372 | 17.030 | 1.00 | 0.00 | 3A7 |
| ATOM | 2134 | OE2 | GLU | 308 | 23.430 | −19.556 | 16.657 | 1.00 | 0.00 | 3A7 |
| ATOM | 2135 | C | GLU | 308 | 20.007 | −18.253 | 20.661 | 1.00 | 0.00 | 3A7 |
| ATOM | 2136 | O | GLU | 308 | 19.119 | −17.424 | 20.814 | 1.00 | 0.00 | 3A7 |
| ATOM | 2137 | N | THR | 309 | 19.741 | −19.531 | 20.323 | 1.00 | 0.00 | 3A7 |
| ATOM | 2138 | CA | THR | 309 | 18.413 | −20.055 | 20.080 | 1.00 | 0.00 | 3A7 |
| ATOM | 2139 | CB | THR | 309 | 18.504 | −21.431 | 19.437 | 1.00 | 0.00 | 3A7 |
| ATOM | 2140 | OG1 | THR | 309 | 17.244 | −21.870 | 18.974 | 1.00 | 0.00 | 3A7 |
| ATOM | 2141 | CG2 | THR | 309 | 19.256 | −22.438 | 20.330 | 1.00 | 0.00 | 3A7 |
| ATOM | 2142 | C | THR | 309 | 17.592 | −20.080 | 21.350 | 1.00 | 0.00 | 3A7 |
| ATOM | 2143 | O | THR | 309 | 16.436 | −19.681 | 21.350 | 1.00 | 0.00 | 3A7 |
| ATOM | 2144 | N | THR | 310 | 18.187 | −20.515 | 22.480 | 1.00 | 0.00 | 3A7 |
| ATOM | 2145 | CA | THR | 310 | 17.519 | −20.567 | 23.760 | 1.00 | 0.00 | 3A7 |
| ATOM | 2146 | CB | THR | 310 | 18.271 | −21.435 | 24.744 | 1.00 | 0.00 | 3A7 |
| ATOM | 2147 | OG1 | THR | 310 | 18.739 | −22.609 | 24.101 | 1.00 | 0.00 | 3A7 |
| ATOM | 2148 | CG2 | THR | 310 | 17.383 | −21.832 | 25.943 | 1.00 | 0.00 | 3A7 |
| ATOM | 2149 | C | THR | 310 | 17.233 | −19.209 | 24.344 | 1.00 | 0.00 | 3A7 |
| ATOM | 2150 | O | THR | 310 | 16.168 | −18.987 | 24.914 | 1.00 | 0.00 | 3A7 |
| ATOM | 2151 | N | SER | 311 | 18.147 | −18.228 | 24.167 | 1.00 | 0.00 | 3A7 |
| ATOM | 2152 | CA | SER | 311 | 17.925 | −16.885 | 24.659 | 1.00 | 0.00 | 3A7 |
| ATOM | 2153 | CB | SER | 311 | 19.171 | −15.993 | 24.620 | 1.00 | 0.00 | 3A7 |
| ATOM | 2154 | OG | SER | 311 | 20.346 | −16.710 | 24.906 | 1.00 | 0.00 | 3A7 |
| ATOM | 2155 | C | SER | 311 | 16.857 | −16.191 | 23.854 | 1.00 | 0.00 | 3A7 |
| ATOM | 2156 | O | SER | 311 | 15.998 | −15.515 | 24.407 | 1.00 | 0.00 | 3A7 |
| ATOM | 2157 | N | SER | 312 | 16.848 | −16.368 | 22.505 | 1.00 | 0.00 | 3A7 |
| ATOM | 2158 | CA | SER | 312 | 15.858 | −15.769 | 21.625 | 1.00 | 0.00 | 3A7 |
| ATOM | 2159 | CB | SER | 312 | 16.203 | −15.894 | 20.128 | 1.00 | 0.00 | 3A7 |
| ATOM | 2160 | OG | SER | 312 | 16.398 | −17.244 | 19.751 | 1.00 | 0.00 | 3A7 |
| ATOM | 2161 | C | SER | 312 | 14.473 | −16.334 | 21.903 | 1.00 | 0.00 | 3A7 |
| ATOM | 2162 | O | SER | 312 | 13.477 | −15.641 | 21.718 | 1.00 | 0.00 | 3A7 |
| ATOM | 2163 | N | VAL | 313 | 14.376 | −17.602 | 22.376 | 1.00 | 0.00 | 3A7 |
| ATOM | 2164 | CA | VAL | 313 | 13.115 | −18.232 | 22.678 | 1.00 | 0.00 | 3A7 |
| ATOM | 2165 | CB | VAL | 313 | 13.262 | −19.732 | 22.736 | 1.00 | 0.00 | 3A7 |
| ATOM | 2166 | CG1 | VAL | 313 | 12.003 | −20.440 | 23.260 | 1.00 | 0.00 | 3A7 |
| ATOM | 2167 | CG2 | VAL | 313 | 13.426 | −20.126 | 21.263 | 1.00 | 0.00 | 3A7 |
| ATOM | 2168 | C | VAL | 313 | 12.555 | −17.712 | 23.971 | 1.00 | 0.00 | 3A7 |
| ATOM | 2169 | O | VAL | 313 | 11.368 | −17.425 | 24.059 | 1.00 | 0.00 | 3A7 |
| ATOM | 2170 | N | LEU | 314 | 13.406 | −17.522 | 25.000 | 1.00 | 0.00 | 3A7 |
| ATOM | 2171 | CA | LEU | 314 | 13.003 | −16.991 | 26.283 | 1.00 | 0.00 | 3A7 |
| ATOM | 2172 | CB | LEU | 314 | 14.178 | −17.030 | 27.279 | 1.00 | 0.00 | 3A7 |
| ATOM | 2173 | CG | LEU | 314 | 14.712 | −18.444 | 27.608 | 1.00 | 0.00 | 3A7 |
| ATOM | 2174 | CD1 | LEU | 314 | 16.160 | −18.400 | 28.135 | 1.00 | 0.00 | 3A7 |
| ATOM | 2175 | CD2 | LEU | 314 | 13.817 | −19.188 | 28.604 | 1.00 | 0.00 | 3A7 |
| ATOM | 2176 | C | LEU | 314 | 12.519 | −15.573 | 26.181 | 1.00 | 0.00 | 3A7 |
| ATOM | 2177 | O | LEU | 314 | 11.490 | −15.204 | 26.736 | 1.00 | 0.00 | 3A7 |
| ATOM | 2178 | N | SER | 315 | 13.252 | −14.741 | 25.411 | 1.00 | 0.00 | 3A7 |
| ATOM | 2179 | CA | SER | 315 | 12.914 | −13.355 | 25.165 | 1.00 | 0.00 | 3A7 |
| ATOM | 2180 | CB | SER | 315 | 14.033 | −12.603 | 24.406 | 1.00 | 0.00 | 3A7 |
| ATOM | 2181 | OG | SER | 315 | 15.234 | −12.612 | 25.166 | 1.00 | 0.00 | 3A7 |
| ATOM | 2182 | C | SER | 315 | 11.625 | −13.209 | 24.374 | 1.00 | 0.00 | 3A7 |
| ATOM | 2183 | O | SER | 315 | 10.814 | −12.336 | 24.676 | 1.00 | 0.00 | 3A7 |
| ATOM | 2184 | N | PHE | 316 | 11.391 | −14.075 | 23.358 | 1.00 | 0.00 | 3A7 |
| ATOM | 2185 | CA | PHE | 316 | 10.164 | −14.069 | 22.587 | 1.00 | 0.00 | 3A7 |
| ATOM | 2186 | CB | PHE | 316 | 10.264 | −14.913 | 21.289 | 1.00 | 0.00 | 3A7 |
| ATOM | 2187 | CG | PHE | 316 | 10.697 | −14.085 | 20.108 | 1.00 | 0.00 | 3A7 |
| ATOM | 2188 | CD1 | PHE | 316 | 11.810 | −14.439 | 19.342 | 1.00 | 0.00 | 3A7 |
| ATOM | 2189 | CD2 | PHE | 316 | 9.933 | −12.985 | 19.709 | 1.00 | 0.00 | 3A7 |
| ATOM | 2190 | CE1 | PHE | 316 | 12.165 | −13.699 | 18.217 | 1.00 | 0.00 | 3A7 |
| ATOM | 2191 | CE2 | PHE | 316 | 10.281 | −12.246 | 18.582 | 1.00 | 0.00 | 3A7 |
| ATOM | 2192 | CZ | PHE | 316 | 11.402 | −12.600 | 17.837 | 1.00 | 0.00 | 3A7 |
| ATOM | 2193 | C | PHE | 316 | 9.015 | −14.618 | 23.406 | 1.00 | 0.00 | 3A7 |
| ATOM | 2194 | O | PHE | 316 | 7.891 | −14.133 | 23.296 | 1.00 | 0.00 | 3A7 |
| ATOM | 2195 | N | ILE | 317 | 9.257 | −15.617 | 24.306 | 1.00 | 0.00 | 3A7 |
| ATOM | 2196 | CA | ILE | 317 | 8.200 | −16.171 | 25.144 | 1.00 | 0.00 | 3A7 |
| ATOM | 2197 | CB | ILE | 317 | 8.522 | −17.418 | 26.022 | 1.00 | 0.00 | 3A7 |
| ATOM | 2198 | CG2 | ILE | 317 | 7.716 | −17.454 | 27.357 | 1.00 | 0.00 | 3A7 |
| ATOM | 2199 | CG1 | ILE | 317 | 8.150 | −18.749 | 25.331 | 1.00 | 0.00 | 3A7 |
| ATOM | 2200 | CD | ILE | 317 | 9.210 | −19.340 | 24.419 | 1.00 | 0.00 | 3A7 |
| ATOM | 2201 | C | ILE | 317 | 7.672 | −15.117 | 26.091 | 1.00 | 0.00 | 3A7 |
| ATOM | 2202 | O | ILE | 317 | 6.464 | −14.997 | 26.274 | 1.00 | 0.00 | 3A7 |
| ATOM | 2203 | N | ILE | 318 | 8.566 | −14.313 | 26.715 | 1.00 | 0.00 | 3A7 |
| ATOM | 2204 | CA | ILE | 318 | 8.192 | −13.252 | 27.635 | 1.00 | 0.00 | 3A7 |
| ATOM | 2205 | CB | ILE | 318 | 9.425 | −12.624 | 28.274 | 1.00 | 0.00 | 3A7 |
| ATOM | 2206 | CG2 | ILE | 318 | 9.076 | −11.357 | 29.092 | 1.00 | 0.00 | 3A7 |
| ATOM | 2207 | CG1 | ILE | 318 | 10.087 | −13.689 | 29.181 | 1.00 | 0.00 | 3A7 |
| ATOM | 2208 | CD | ILE | 318 | 11.481 | −13.295 | 29.665 | 1.00 | 0.00 | 3A7 |
| ATOM | 2209 | C | ILE | 318 | 7.339 | −12.213 | 26.942 | 1.00 | 0.00 | 3A7 |
| ATOM | 2210 | O | ILE | 318 | 6.296 | −11.802 | 27.441 | 1.00 | 0.00 | 3A7 |
| ATOM | 2211 | N | TYR | 319 | 7.731 | −11.823 | 25.716 | 1.00 | 0.00 | 3A7 |
| ATOM | 2212 | CA | TYR | 319 | 6.987 | −10.893 | 24.906 | 1.00 | 0.00 | 3A7 |
| ATOM | 2213 | CB | TYR | 319 | 7.729 | −10.658 | 23.571 | 1.00 | 0.00 | 3A7 |
| ATOM | 2214 | CG | TYR | 319 | 6.940 | −9.879 | 22.557 | 1.00 | 0.00 | 3A7 |
| ATOM | 2215 | CD1 | TYR | 319 | 6.414 | −8.628 | 22.863 | 1.00 | 0.00 | 3A7 |
| ATOM | 2216 | CD2 | TYR | 319 | 6.693 | −10.438 | 21.301 | 1.00 | 0.00 | 3A7 |
| ATOM | 2217 | CE1 | TYR | 319 | 5.597 | −7.976 | 21.949 | 1.00 | 0.00 | 3A7 |
| ATOM | 2218 | CE2 | TYR | 319 | 5.898 | −9.773 | 20.375 | 1.00 | 0.00 | 3A7 |
| ATOM | 2219 | CZ | TYR | 319 | 5.336 | −8.544 | 20.704 | 1.00 | 0.00 | 3A7 |
| ATOM | 2220 | OH | TYR | 319 | 4.491 | −7.887 | 19.783 | 1.00 | 0.00 | 3A7 |
| ATOM | 2221 | C | TYR | 319 | 5.568 | −11.359 | 24.631 | 1.00 | 0.00 | 3A7 |
| ATOM | 2222 | O | TYR | 319 | 4.622 | −10.588 | 24.752 | 1.00 | 0.00 | 3A7 |
| ATOM | 2223 | N | GLU | 320 | 5.404 | −12.651 | 24.277 | 1.00 | 0.00 | 3A7 |
| ATOM | 2224 | CA | GLU | 320 | 4.118 | −13.205 | 23.952 | 1.00 | 0.00 | 3A7 |
| ATOM | 2225 | CB | GLU | 320 | 4.190 | −14.545 | 23.220 | 1.00 | 0.00 | 3A7 |
| ATOM | 2226 | CG | GLU | 320 | 4.824 | −14.416 | 21.829 | 1.00 | 0.00 | 3A7 |
| ATOM | 2227 | CD | GLU | 320 | 3.795 | −13.768 | 20.915 | 1.00 | 0.00 | 3A7 |
| ATOM | 2228 | OE1 | GLU | 320 | 4.068 | −12.634 | 20.435 | 1.00 | 0.00 | 3A7 |
| ATOM | 2229 | OE2 | GLU | 320 | 2.723 | −14.384 | 20.681 | 1.00 | 0.00 | 3A7 |
| ATOM | 2230 | C | GLU | 320 | 3.209 | −13.345 | 25.140 | 1.00 | 0.00 | 3A7 |
| ATOM | 2231 | O | GLU | 320 | 2.018 | −13.087 | 25.041 | 1.00 | 0.00 | 3A7 |
| ATOM | 2232 | N | LEU | 321 | 3.746 | −13.705 | 26.321 | 1.00 | 0.00 | 3A7 |
| ATOM | 2233 | CA | LEU | 321 | 2.972 | −13.799 | 27.537 | 1.00 | 0.00 | 3A7 |
| ATOM | 2234 | CB | LEU | 321 | 3.805 | −14.427 | 28.662 | 1.00 | 0.00 | 3A7 |
| ATOM | 2235 | CG | LEU | 321 | 4.011 | −15.941 | 28.472 | 1.00 | 0.00 | 3A7 |
| ATOM | 2236 | CD1 | LEU | 321 | 5.072 | −16.456 | 29.453 | 1.00 | 0.00 | 3A7 |
| ATOM | 2237 | CD2 | LEU | 321 | 2.687 | −16.706 | 28.641 | 1.00 | 0.00 | 3A7 |
| ATOM | 2238 | C | LEU | 321 | 2.505 | −12.447 | 28.007 | 1.00 | 0.00 | 3A7 |
| ATOM | 2239 | O | LEU | 321 | 1.395 | −12.302 | 28.509 | 1.00 | 0.00 | 3A7 |
| ATOM | 2240 | N | ALA | 322 | 3.360 | −11.421 | 27.837 | 1.00 | 0.00 | 3A7 |
| ATOM | 2241 | CA | ALA | 322 | 3.065 | −10.068 | 28.220 | 1.00 | 0.00 | 3A7 |
| ATOM | 2242 | CB | ALA | 322 | 4.351 | −9.229 | 28.225 | 1.00 | 0.00 | 3A7 |
| ATOM | 2243 | C | ALA | 322 | 2.044 | −9.469 | 27.279 | 1.00 | 0.00 | 3A7 |
| ATOM | 2244 | O | ALA | 322 | 1.166 | −8.702 | 27.668 | 1.00 | 0.00 | 3A7 |
| ATOM | 2245 | N | THR | 323 | 2.110 | −9.863 | 25.996 | 1.00 | 0.00 | 3A7 |
| ATOM | 2246 | CA | THR | 323 | 1.174 | −9.402 | 25.006 | 1.00 | 0.00 | 3A7 |
| ATOM | 2247 | CB | THR | 323 | 1.714 | −9.509 | 23.612 | 1.00 | 0.00 | 3A7 |
| ATOM | 2248 | OG1 | THR | 323 | 2.224 | −10.786 | 23.282 | 1.00 | 0.00 | 3A7 |
| ATOM | 2249 | CG2 | THR | 323 | 2.782 | −8.417 | 23.418 | 1.00 | 0.00 | 3A7 |
| ATOM | 2250 | C | THR | 323 | −0.164 | −10.105 | 25.063 | 1.00 | 0.00 | 3A7 |
| ATOM | 2251 | O | THR | 323 | −1.132 | −9.627 | 24.486 | 1.00 | 0.00 | 3A7 |
| ATOM | 2252 | N | HIS | 324 | −0.258 | −11.241 | 25.774 | 1.00 | 0.00 | 3A7 |
| ATOM | 2253 | CA | HIS | 324 | −1.475 | −12.018 | 25.882 | 1.00 | 0.00 | 3A7 |
| ATOM | 2254 | ND1 | HIS | 324 | −2.250 | −12.554 | 22.767 | 1.00 | 0.00 | 3A7 |
| ATOM | 2255 | CG | HIS | 324 | −1.279 | −13.139 | 23.576 | 1.00 | 0.00 | 3A7 |
| ATOM | 2256 | CB | HIS | 324 | −1.248 | −13.311 | 25.087 | 1.00 | 0.00 | 3A7 |
| ATOM | 2257 | NE2 | HIS | 324 | −0.359 | −12.447 | 21.617 | 1.00 | 0.00 | 3A7 |
| ATOM | 2258 | CD2 | HIS | 324 | −0.126 | −13.016 | 22.856 | 1.00 | 0.00 | 3A7 |
| ATOM | 2259 | CE1 | HIS | 324 | −1.648 | −12.187 | 21.610 | 1.00 | 0.00 | 3A7 |
| ATOM | 2260 | C | HIS | 324 | −1.699 | −12.327 | 27.334 | 1.00 | 0.00 | 3A7 |
| ATOM | 2261 | O | HIS | 324 | −1.410 | −13.448 | 21.764 | 1.00 | 0.00 | 3A7 |
| ATOM | 2262 | N | PRO | 325 | −2.246 | −11.398 | 28.139 | 1.00 | 0.00 | 3A7 |
| ATOM | 2263 | CA | PRO | 325 | −2.362 | −11.530 | 29.587 | 1.00 | 0.00 | 3A7 |
| ATOM | 2264 | CD | PRO | 325 | −2.664 | −10.073 | 27.697 | 1.00 | 0.00 | 3A7 |
| ATOM | 2265 | CB | PRO | 325 | −3.005 | −10.217 | 30.049 | 1.00 | 0.00 | 3A7 |
| ATOM | 2266 | CG | PRO | 325 | −2.634 | −9.212 | 28.959 | 1.00 | 0.00 | 3A7 |
| ATOM | 2267 | C | PRO | 325 | −3.219 | −12.703 | 30.001 | 1.00 | 0.00 | 3A7 |
| ATOM | 2268 | O | PRO | 325 | −2.994 | −13.255 | 31.072 | 1.00 | 0.00 | 3A7 |
| ATOM | 2269 | N | ASP | 326 | −4.183 | −13.144 | 29.162 | 1.00 | 0.00 | 3A7 |
| ATOM | 2270 | CA | ASP | 326 | −5.016 | −14.290 | 29.448 | 1.00 | 0.00 | 3A7 |
| ATOM | 2271 | CB | ASP | 326 | −6.108 | −14.480 | 28.372 | 1.00 | 0.00 | 3A7 |
| ATOM | 2272 | CG | ASP | 326 | −7.091 | −13.305 | 28.363 | 1.00 | 0.00 | 3A7 |
| ATOM | 2273 | OD1 | ASP | 326 | −7.016 | −12.435 | 29.271 | 1.00 | 0.00 | 3A7 |
| ATOM | 2274 | OD2 | ASP | 326 | −7.946 | −13.276 | 27.437 | 1.00 | 0.00 | 3A7 |
| ATOM | 2275 | C | ASP | 326 | −4.191 | −15.557 | 29.518 | 1.00 | 0.00 | 3A7 |
| ATOM | 2276 | O | ASP | 326 | −4.391 | −16.408 | 30.379 | 1.00 | 0.00 | 3A7 |
| ATOM | 2277 | N | VAL | 327 | −3.192 | −15.680 | 28.617 | 1.00 | 0.00 | 3A7 |
| ATOM | 2278 | CA | VAL | 327 | −2.308 | −16.822 | 28.551 | 1.00 | 0.00 | 3A7 |
| ATOM | 2279 | CB | VAL | 327 | −1.522 | −16.855 | 27.249 | 1.00 | 0.00 | 3A7 |
| ATOM | 2280 | CG1 | VAL | 327 | −0.634 | −18.114 | 27.189 | 1.00 | 0.00 | 3A7 |
| ATOM | 2281 | CG2 | VAL | 327 | −2.526 | −16.830 | 26.078 | 1.00 | 0.00 | 3A7 |
| ATOM | 2282 | C | VAL | 327 | −1.357 | −16.811 | 29.718 | 1.00 | 0.00 | 3A7 |
| ATOM | 2283 | O | VAL | 327 | −1.126 | −17.833 | 30.353 | 1.00 | 0.00 | 3A7 |
| ATOM | 2284 | N | GLN | 328 | −0.822 | −15.622 | 30.057 | 1.00 | 0.00 | 3A7 |
| ATOM | 2285 | CA | GLN | 328 | 0.054 | −15.440 | 31.190 | 1.00 | 0.00 | 3A7 |
| ATOM | 2286 | CB | GLN | 328 | 0.551 | −13.994 | 31.280 | 1.00 | 0.00 | 3A7 |
| ATOM | 2287 | CG | GLN | 328 | 1.927 | −13.874 | 31.956 | 1.00 | 0.00 | 3A7 |
| ATOM | 2288 | CD | GLN | 328 | 2.430 | −12.432 | 31.850 | 1.00 | 0.00 | 3A7 |
| ATOM | 2289 | OE1 | GLN | 328 | 1.677 | −11.512 | 31.507 | 1.00 | 0.00 | 3A7 |
| ATOM | 2290 | NE2 | GLN | 328 | 3.749 | −12.247 | 32.162 | 1.00 | 0.00 | 3A7 |
| ATOM | 2291 | C | GLN | 328 | −0.565 | −15.809 | 32.500 | 1.00 | 0.00 | 3A7 |
| ATOM | 2292 | O | GLN | 328 | 0.057 | −16.448 | 33.339 | 1.00 | 0.00 | 3A7 |
| ATOM | 2293 | N | GLN | 329 | −1.847 | −15.438 | 32.692 | 1.00 | 0.00 | 3A7 |
| ATOM | 2294 | CA | GLN | 329 | −2.599 | −15.749 | 33.879 | 1.00 | 0.00 | 3A7 |
| ATOM | 2295 | CB | GLN | 329 | −3.961 | −15.032 | 33.870 | 1.00 | 0.00 | 3A7 |
| ATOM | 2296 | CG | GLN | 329 | −3.832 | −13.525 | 34.148 | 1.00 | 0.00 | 3A7 |
| ATOM | 2297 | CD | GLN | 329 | −5.215 | −12.882 | 34.023 | 1.00 | 0.00 | 3A7 |
| ATOM | 2298 | OE1 | GLN | 329 | −6.121 | −13.175 | 34.813 | 1.00 | 0.00 | 3A7 |
| ATOM | 2299 | NE2 | GLN | 329 | −5.369 | −11.987 | 33.000 | 1.00 | 0.00 | 3A7 |
| ATOM | 2300 | C | GLN | 329 | −2.821 | −17.226 | 34.022 | 1.00 | 0.00 | 3A7 |
| ATOM | 2301 | O | GLN | 329 | −2.653 | −17.780 | 35.102 | 1.00 | 0.00 | 3A7 |
| ATOM | 2302 | N | LYS | 330 | −3.153 | −17.913 | 32.907 | 1.00 | 0.00 | 3A7 |
| ATOM | 2303 | CA | LYS | 330 | −3.370 | −19.343 | 32.883 | 1.00 | 0.00 | 3A7 |
| ATOM | 2304 | CB | LYS | 330 | −3.917 | −19.754 | 31.503 | 1.00 | 0.00 | 3A7 |
| ATOM | 2305 | CG | LYS | 330 | −4.508 | −21.169 | 31.446 | 1.00 | 0.00 | 3A7 |
| ATOM | 2306 | CD | LYS | 330 | −5.246 | −21.428 | 30.124 | 1.00 | 0.00 | 3A7 |
| ATOM | 2307 | CE | LYS | 330 | −5.927 | −22.799 | 30.054 | 1.00 | 0.00 | 3A7 |
| ATOM | 2308 | NZ | LYS | 330 | −6.952 | −22.943 | 31.113 | 1.00 | 0.00 | 3A7 |
| ATOM | 2309 | C | LYS | 330 | −2.097 | −20.090 | 33.241 | 1.00 | 0.00 | 3A7 |
| ATOM | 2310 | O | LYS | 330 | −2.090 | −21.027 | 34.040 | 1.00 | 0.00 | 3A7 |
| ATOM | 2311 | N | VAL | 331 | −0.946 | −19.618 | 32.704 | 1.00 | 0.00 | 3A7 |
| ATOM | 2312 | CA | VAL | 331 | 0.363 | −20.189 | 32.957 | 1.00 | 0.00 | 3A7 |
| ATOM | 2313 | CB | VAL | 331 | 1.419 | −19.605 | 32.030 | 1.00 | 0.00 | 3A7 |
| ATOM | 2314 | CG1 | VAL | 331 | 2.843 | −20.076 | 32.382 | 1.00 | 0.00 | 3A7 |
| ATOM | 2315 | CG2 | VAL | 331 | 1.080 | −20.072 | 30.601 | 1.00 | 0.00 | 3A7 |
| ATOM | 2316 | C | VAL | 331 | 0.782 | −19.992 | 34.390 | 1.00 | 0.00 | 3A7 |
| ATOM | 2317 | O | VAL | 331 | 1.274 | −20.919 | 35.023 | 1.00 | 0.00 | 3A7 |
| ATOM | 2318 | N | GLN | 332 | 0.561 | −18.783 | 34.951 | 1.00 | 0.00 | 3A7 |
| ATOM | 2319 | CA | GLN | 332 | 0.896 | −18.455 | 36.319 | 1.00 | 0.00 | 3A7 |
| ATOM | 2320 | CB | GLN | 332 | 0.668 | −16.968 | 36.628 | 1.00 | 0.00 | 3A7 |
| ATOM | 2321 | CG | GLN | 332 | 1.756 | −16.072 | 36.011 | 1.00 | 0.00 | 3A7 |
| ATOM | 2322 | CD | GLN | 332 | 1.457 | −14.601 | 36.311 | 1.00 | 0.00 | 3A7 |
| ATOM | 2323 | OE1 | GLN | 332 | 2.196 | −13.947 | 37.057 | 1.00 | 0.00 | 3A7 |
| ATOM | 2324 | NE2 | GLN | 332 | 0.350 | −14.079 | 35.701 | 1.00 | 0.00 | 3A7 |
| ATOM | 2325 | C | GLN | 332 | 0.082 | −19.234 | 37.311 | 1.00 | 0.00 | 3A7 |
| ATOM | 2326 | O | GLN | 332 | 0.592 | −19.634 | 38.351 | 1.00 | 0.00 | 3A7 |
| ATOM | 2327 | N | LYS | 333 | −1.200 | −19.512 | 36.991 | 1.00 | 0.00 | 3A7 |
| ATOM | 2328 | CA | LYS | 333 | −2.087 | −20.320 | 37.797 | 1.00 | 0.00 | 3A7 |
| ATOM | 2329 | CB | LYS | 333 | −3.508 | −20.312 | 37.204 | 1.00 | 0.00 | 3A7 |
| ATOM | 2330 | CG | LYS | 333 | −4.571 | −21.052 | 38.035 | 1.00 | 0.00 | 3A7 |
| ATOM | 2331 | CD | LYS | 333 | −5.986 | −20.945 | 37.441 | 1.00 | 0.00 | 3A7 |
| ATOM | 2332 | CE | LYS | 333 | −6.125 | −21.617 | 36.068 | 1.00 | 0.00 | 3A7 |
| ATOM | 2333 | NZ | LYS | 333 | −7.518 | −21.516 | 35.576 | 1.00 | 0.00 | 3A7 |
| ATOM | 2334 | C | LYS | 333 | −1.600 | −21.746 | 37.882 | 1.00 | 0.00 | 3A7 |
| ATOM | 2335 | O | LYS | 333 | −1.547 | −22.344 | 38.954 | 1.00 | 0.00 | 3A7 |
| ATOM | 2336 | N | GLU | 334 | −1.171 | −22.295 | 36.728 | 1.00 | 0.00 | 3A7 |
| ATOM | 2337 | CA | GLU | 334 | −0.533 | −23.579 | 36.649 | 1.00 | 0.00 | 3A7 |
| ATOM | 2338 | CB | GLU | 334 | −0.149 | −23.952 | 35.224 | 1.00 | 0.00 | 3A7 |
| ATOM | 2339 | CG | GLU | 334 | −0.038 | −25.481 | 35.122 | 1.00 | 0.00 | 3A7 |
| ATOM | 2340 | CD | GLU | 334 | 0.087 | −25.922 | 33.672 | 1.00 | 0.00 | 3A7 |
| ATOM | 2341 | OE1 | GLU | 334 | −0.736 | −26.779 | 33.251 | 1.00 | 0.00 | 3A7 |
| ATOM | 2342 | OE2 | GLU | 334 | 1.007 | −25.424 | 32.971 | 1.00 | 0.00 | 3A7 |
| ATOM | 2343 | C | GLU | 334 | 0.701 | −23.722 | 37.472 | 1.00 | 0.00 | 3A7 |
| ATOM | 2344 | O | GLU | 334 | 0.825 | −24.667 | 38.236 | 1.00 | 0.00 | 3A7 |
| ATOM | 2345 | N | ILE | 335 | 1.627 | −22.744 | 37.370 | 1.00 | 0.00 | 3A7 |
| ATOM | 2346 | CA | ILE | 335 | 2.874 | −22.692 | 38.098 | 1.00 | 0.00 | 3A7 |
| ATOM | 2347 | CB | ILE | 335 | 3.672 | −21.474 | 37.679 | 1.00 | 0.00 | 3A7 |
| ATOM | 2348 | CG2 | ILE | 335 | 4.884 | −21.230 | 38.599 | 1.00 | 0.00 | 3A7 |
| ATOM | 2349 | CG1 | ILE | 335 | 4.128 | −21.660 | 36.215 | 1.00 | 0.00 | 3A7 |
| ATOM | 2350 | CD | ILE | 335 | 4.634 | −20.360 | 35.597 | 1.00 | 0.00 | 3A7 |
| ATOM | 2351 | C | ILE | 335 | 2.643 | −22.684 | 39.587 | 1.00 | 0.00 | 3A7 |
| ATOM | 2352 | O | ILE | 335 | 3.219 | −23.480 | 40.320 | 1.00 | 0.00 | 3A7 |
| ATOM | 2353 | N | ASP | 336 | 1.727 | −21.822 | 40.071 | 1.00 | 0.00 | 3A7 |
| ATOM | 2354 | CA | ASP | 336 | 1.416 | −21.706 | 41.476 | 1.00 | 0.00 | 3A7 |
| ATOM | 2355 | CB | ASP | 336 | 0.411 | −20.565 | 41.750 | 1.00 | 0.00 | 3A7 |
| ATOM | 2356 | CG | ASP | 336 | 1.021 | −19.199 | 41.420 | 1.00 | 0.00 | 3A7 |
| ATOM | 2357 | OD1 | ASP | 336 | 2.243 | −19.127 | 41.123 | 1.00 | 0.00 | 3A7 |
| ATOM | 2358 | OD2 | ASP | 336 | 0.255 | −18.199 | 41.470 | 1.00 | 0.00 | 3A7 |
| ATOM | 2359 | C | ASP | 336 | 0.838 | −22.996 | 42.010 | 1.00 | 0.00 | 3A7 |
| ATOM | 2360 | O | ASP | 336 | 1.183 | −23.420 | 43.106 | 1.00 | 0.00 | 3A7 |
| ATOM | 2361 | N | THR | 337 | −0.022 | −23.674 | 41.213 | 1.00 | 0.00 | 3A7 |
| ATOM | 2362 | CA | THR | 337 | −0.701 | −24.903 | 41.571 | 1.00 | 0.00 | 3A7 |
| ATOM | 2363 | CB | THR | 337 | −1.854 | −25.156 | 40.609 | 1.00 | 0.00 | 3A7 |
| ATOM | 2364 | OG1 | THR | 337 | −2.791 | −24.092 | 40.721 | 1.00 | 0.00 | 3A7 |
| ATOM | 2365 | CG2 | THR | 337 | −2.601 | −26.473 | 40.896 | 1.00 | 0.00 | 3A7 |
| ATOM | 2366 | C | THR | 337 | 0.216 | −26.114 | 41.595 | 1.00 | 0.00 | 3A7 |
| ATOM | 2367 | O | THR | 337 | 0.055 | −27.000 | 42.433 | 1.00 | 0.00 | 3A7 |
| ATOM | 2368 | N | VAL | 338 | 1.202 | −26.189 | 40.667 | 1.00 | 0.00 | 3A7 |
| ATOM | 2369 | CA | VAL | 338 | 2.055 | −27.349 | 40.503 | 1.00 | 0.00 | 3A7 |
| ATOM | 2370 | CB | VAL | 338 | 2.466 | −27.564 | 39.051 | 1.00 | 0.00 | 3A7 |
| ATOM | 2371 | CG1 | VAL | 338 | 3.403 | −28.783 | 38.900 | 1.00 | 0.00 | 3A7 |
| ATOM | 2372 | CG2 | VAL | 338 | 1.182 | −27.789 | 38.226 | 1.00 | 0.00 | 3A7 |
| ATOM | 2373 | C | VAL | 338 | 3.261 | −27.232 | 41.404 | 1.00 | 0.00 | 3A7 |
| ATOM | 2374 | O | VAL | 338 | 3.589 | −28.178 | 42.113 | 1.00 | 0.00 | 3A7 |
| ATOM | 2375 | N | LEU | 339 | 3.950 | −26.065 | 41.421 | 1.00 | 0.00 | 3A7 |
| ATOM | 2376 | CA | LEU | 339 | 5.053 | −25.835 | 42.331 | 1.00 | 0.00 | 3A7 |
| ATOM | 2377 | CB | LEU | 339 | 6.213 | −25.014 | 41.736 | 1.00 | 0.00 | 3A7 |
| ATOM | 2378 | CG | LEU | 339 | 7.082 | −25.819 | 40.766 | 1.00 | 0.00 | 3A7 |
| ATOM | 2379 | CD1 | LEU | 339 | 8.159 | −24.904 | 40.176 | 1.00 | 0.00 | 3A7 |
| ATOM | 2380 | CD2 | LEU | 339 | 7.715 | −27.051 | 41.439 | 1.00 | 0.00 | 3A7 |
| ATOM | 2381 | C | LEU | 339 | 4.552 | −25.038 | 43.521 | 1.00 | 0.00 | 3A7 |
| ATOM | 2382 | O | LEU | 339 | 4.197 | −23.878 | 43.325 | 1.00 | 0.00 | 3A7 |
| ATOM | 2383 | N | PRO | 340 | 4.486 | −25.565 | 44.752 | 1.00 | 0.00 | 3A7 |
| ATOM | 2384 | CA | PRO | 340 | 3.973 | −24.851 | 45.907 | 1.00 | 0.00 | 3A7 |
| ATOM | 2385 | CD | PRO | 340 | 4.660 | −26.986 | 45.025 | 1.00 | 0.00 | 3A7 |
| ATOM | 2386 | CB | PRO | 340 | 3.483 | −25.968 | 46.840 | 1.00 | 0.00 | 3A7 |
| ATOM | 2387 | CG | PRO | 340 | 4.398 | −27.154 | 46.523 | 1.00 | 0.00 | 3A7 |
| ATOM | 2388 | C | PRO | 340 | 5.084 | −24.042 | 46.550 | 1.00 | 0.00 | 3A7 |
| ATOM | 2389 | O | PRO | 340 | 6.241 | −24.463 | 46.512 | 1.00 | 0.00 | 3A7 |
| ATOM | 2390 | N | ASN | 341 | 4.716 | −22.893 | 47.182 | 1.00 | 0.00 | 3A7 |
| ATOM | 2391 | CA | ASN | 341 | 5.537 | −22.073 | 48.065 | 1.00 | 0.00 | 3A7 |
| ATOM | 2392 | CB | ASN | 341 | 6.167 | −22.872 | 49.244 | 1.00 | 0.00 | 3A7 |
| ATOM | 2393 | CG | ASN | 341 | 5.072 | −23.614 | 50.023 | 1.00 | 0.00 | 3A7 |
| ATOM | 2394 | OD1 | ASN | 341 | 5.051 | −24.850 | 50.059 | 1.00 | 0.00 | 3A7 |
| ATOM | 2395 | ND2 | ASN | 341 | 4.155 | −22.826 | 50.663 | 1.00 | 0.00 | 3A7 |
| ATOM | 2396 | C | ASN | 341 | 6.625 | −21.330 | 47.312 | 1.00 | 0.00 | 3A7 |
| ATOM | 2397 | O | ASN | 341 | 6.423 | −20.897 | 46.178 | 1.00 | 0.00 | 3A7 |
| ATOM | 2398 | N | LYS | 342 | 7.813 | −21.175 | 47.953 | 1.00 | 0.00 | 3A7 |
| ATOM | 2399 | CA | LYS | 342 | 8.980 | −20.529 | 47.396 | 1.00 | 0.00 | 3A7 |
| ATOM | 2400 | CB | LYS | 342 | 9.578 | −19.456 | 48.339 | 1.00 | 0.00 | 3A7 |
| ATOM | 2401 | CG | LYS | 342 | 8.601 | −18.309 | 48.647 | 1.00 | 0.00 | 3A7 |
| ATOM | 2402 | CD | LYS | 342 | 9.210 | −17.205 | 49.527 | 1.00 | 0.00 | 3A7 |
| ATOM | 2403 | CE | LYS | 342 | 9.570 | −17.674 | 50.944 | 1.00 | 0.00 | 3A7 |
| ATOM | 2404 | NZ | LYS | 342 | 10.133 | −16.560 | 51.741 | 1.00 | 0.00 | 3A7 |
| ATOM | 2405 | C | LYS | 342 | 10.016 | −21.594 | 47.146 | 1.00 | 0.00 | 3A7 |
| ATOM | 2406 | O | LYS | 342 | 11.181 | −21.442 | 47.515 | 1.00 | 0.00 | 3A7 |
| ATOM | 2407 | N | ALA | 343 | 9.595 | −22.711 | 46.501 | 1.00 | 0.00 | 3A7 |
| ATOM | 2408 | CA | ALA | 343 | 10.434 | −23.839 | 46.171 | 1.00 | 0.00 | 3A7 |
| ATOM | 2409 | CB | ALA | 343 | 9.750 | −25.189 | 46.468 | 1.00 | 0.00 | 3A7 |
| ATOM | 2410 | C | ALA | 343 | 10.737 | −23.763 | 44.700 | 1.00 | 0.00 | 3A7 |
| ATOM | 2411 | O | ALA | 343 | 9.824 | −23.520 | 43.911 | 1.00 | 0.00 | 3A7 |
| ATOM | 2412 | N | PRO | 344 | 11.980 | −24.000 | 44.274 | 1.00 | 0.00 | 3A7 |
| ATOM | 2413 | CA | PRO | 344 | 12.353 | −24.110 | 42.875 | 1.00 | 0.00 | 3A7 |
| ATOM | 2414 | CD | PRO | 344 | 13.149 | −23.771 | 45.118 | 1.00 | 0.00 | 3A7 |
| ATOM | 2415 | CB | PRO | 344 | 13.894 | −24.072 | 42.880 | 1.00 | 0.00 | 3A7 |
| ATOM | 2416 | CG | PRO | 344 | 14.248 | −23.318 | 44.160 | 1.00 | 0.00 | 3A7 |
| ATOM | 2417 | C | PRO | 344 | 11.838 | −25.414 | 42.274 | 1.00 | 0.00 | 3A7 |
| ATOM | 2418 | O | PRO | 344 | 11.420 | −26.289 | 43.033 | 1.00 | 0.00 | 3A7 |
| ATOM | 2419 | N | PRO | 345 | 11.880 | −25.589 | 40.953 | 1.00 | 0.00 | 3A7 |
| ATOM | 2420 | CA | PRO | 345 | 11.325 | −26.737 | 40.270 | 1.00 | 0.00 | 3A7 |
| ATOM | 2421 | CD | PRO | 345 | 12.098 | −24.479 | 40.043 | 1.00 | 0.00 | 3A7 |
| ATOM | 2422 | CB | PRO | 345 | 11.402 | −26.382 | 38.778 | 1.00 | 0.00 | 3A7 |
| ATOM | 2423 | CG | PRO | 345 | 11.412 | −24.857 | 38.731 | 1.00 | 0.00 | 3A7 |
| ATOM | 2424 | C | PRO | 345 | 12.117 | −27.996 | 40.521 | 1.00 | 0.00 | 3A7 |
| ATOM | 2425 | O | PRO | 345 | 13.329 | −27.934 | 40.723 | 1.00 | 0.00 | 3A7 |
| ATOM | 2426 | N | THR | 346 | 11.432 | −29.156 | 40.428 | 1.00 | 0.00 | 3A7 |
| ATOM | 2427 | CA | THR | 346 | 12.073 | −30.434 | 40.286 | 1.00 | 0.00 | 3A7 |
| ATOM | 2428 | CB | THR | 346 | 11.485 | −31.522 | 41.180 | 1.00 | 0.00 | 3A7 |
| ATOM | 2429 | OG1 | THR | 346 | 10.071 | −31.633 | 41.047 | 1.00 | 0.00 | 3A7 |
| ATOM | 2430 | CG2 | THR | 346 | 11.830 | −31.183 | 42.644 | 1.00 | 0.00 | 3A7 |
| ATOM | 2431 | C | THR | 346 | 11.920 | −30.751 | 38.827 | 1.00 | 0.00 | 3A7 |
| ATOM | 2432 | O | THR | 346 | 11.346 | −29.983 | 38.054 | 1.00 | 0.00 | 3A7 |
| ATOM | 2433 | N | TYR | 347 | 12.422 | −31.922 | 38.405 | 1.00 | 0.00 | 3A7 |
| ATOM | 2434 | CA | TYR | 347 | 12.345 | −32.298 | 37.022 | 1.00 | 0.00 | 3A7 |
| ATOM | 2435 | CB | TYR | 347 | 13.345 | −33.383 | 36.599 | 1.00 | 0.00 | 3A7 |
| ATOM | 2436 | CG | TYR | 347 | 13.830 | −34.221 | 37.758 | 1.00 | 0.00 | 3A7 |
| ATOM | 2437 | CD1 | TYR | 347 | 13.226 | −35.455 | 38.007 | 1.00 | 0.00 | 3A7 |
| ATOM | 2438 | CD2 | TYR | 347 | 14.880 | −33.809 | 38.584 | 1.00 | 0.00 | 3A7 |
| ATOM | 2439 | CE1 | TYR | 347 | 13.653 | −36.258 | 39.060 | 1.00 | 0.00 | 3A7 |
| ATOM | 2440 | CE2 | TYR | 347 | 15.310 | −34.609 | 39.638 | 1.00 | 0.00 | 3A7 |
| ATOM | 2441 | CZ | TYR | 347 | 14.697 | −35.835 | 39.877 | 1.00 | 0.00 | 3A7 |
| ATOM | 2442 | OH | TYR | 347 | 15.133 | −36.649 | 40.945 | 1.00 | 0.00 | 3A7 |
| ATOM | 2443 | C | TYR | 347 | 10.986 | −32.842 | 36.741 | 1.00 | 0.00 | 3A7 |
| ATOM | 2444 | O | TYR | 347 | 10.471 | −32.653 | 35.655 | 1.00 | 0.00 | 3A7 |
| ATOM | 2445 | N | ASP | 348 | 10.354 | −33.487 | 37.746 | 1.00 | 0.00 | 3A7 |
| ATOM | 2446 | CA | ASP | 348 | 9.033 | −34.072 | 37.677 | 1.00 | 0.00 | 3A7 |
| ATOM | 2447 | CB | ASP | 348 | 8.708 | −34.785 | 38.996 | 1.00 | 0.00 | 3A7 |
| ATOM | 2448 | CG | ASP | 348 | 9.678 | −35.950 | 39.163 | 1.00 | 0.00 | 3A7 |
| ATOM | 2449 | OD1 | ASP | 348 | 9.665 | −36.846 | 38.277 | 1.00 | 0.00 | 3A7 |
| ATOM | 2450 | OD2 | ASP | 348 | 10.439 | −35.959 | 40.167 | 1.00 | 0.00 | 3A7 |
| ATOM | 2451 | C | ASP | 348 | 7.939 | −33.064 | 37.424 | 1.00 | 0.00 | 3A7 |
| ATOM | 2452 | O | ASP | 348 | 6.884 | −33.393 | 36.890 | 1.00 | 0.00 | 3A7 |
| ATOM | 2453 | N | THR | 349 | 8.224 | −31.784 | 37.764 | 1.00 | 0.00 | 3A7 |
| ATOM | 2454 | CA | THR | 349 | 7.414 | −30.615 | 37.512 | 1.00 | 0.00 | 3A7 |
| ATOM | 2455 | CB | THR | 349 | 8.047 | −29.394 | 38.153 | 1.00 | 0.00 | 3A7 |
| ATOM | 2456 | OG1 | THR | 349 | 8.345 | −29.682 | 39.512 | 1.00 | 0.00 | 3A7 |
| ATOM | 2457 | CG2 | THR | 349 | 7.118 | −28.168 | 38.103 | 1.00 | 0.00 | 3A7 |
| ATOM | 2458 | C | THR | 349 | 7.246 | −30.355 | 36.030 | 1.00 | 0.00 | 3A7 |
| ATOM | 2459 | O | THR | 349 | 6.224 | −29.855 | 35.582 | 1.00 | 0.00 | 3A7 |
| ATOM | 2460 | N | VAL | 350 | 8.243 | −30.735 | 35.203 | 1.00 | 0.00 | 3A7 |
| ATOM | 2461 | CA | VAL | 350 | 8.261 | −30.585 | 33.760 | 1.00 | 0.00 | 3A7 |
| ATOM | 2462 | CB | VAL | 350 | 9.589 | −31.111 | 33.245 | 1.00 | 0.00 | 3A7 |
| ATOM | 2463 | CG1 | VAL | 350 | 9.700 | −31.257 | 31.710 | 1.00 | 0.00 | 3A7 |
| ATOM | 2464 | CG2 | VAL | 350 | 10.704 | −30.188 | 33.784 | 1.00 | 0.00 | 3A7 |
| ATOM | 2465 | C | VAL | 350 | 7.128 | −31.305 | 33.054 | 1.00 | 0.00 | 3A7 |
| ATOM | 2466 | O | VAL | 350 | 6.691 | −30.900 | 31.980 | 1.00 | 0.00 | 3A7 |
| ATOM | 2467 | N | LEU | 351 | 6.619 | −32.397 | 33.667 | 1.00 | 0.00 | 3A7 |
| ATOM | 2468 | CA | LEU | 351 | 5.583 | −33.220 | 33.098 | 1.00 | 0.00 | 3A7 |
| ATOM | 2469 | CB | LEU | 351 | 5.689 | −34.682 | 33.599 | 1.00 | 0.00 | 3A7 |
| ATOM | 2470 | CG | LEU | 351 | 7.076 | −35.324 | 33.359 | 1.00 | 0.00 | 3A7 |
| ATOM | 2471 | CD1 | LEU | 351 | 7.162 | −36.712 | 34.024 | 1.00 | 0.00 | 3A7 |
| ATOM | 2472 | CD2 | LEU | 351 | 7.441 | −35.402 | 31.865 | 1.00 | 0.00 | 3A7 |
| ATOM | 2473 | C | LEU | 351 | 4.203 | −32.710 | 33.432 | 1.00 | 0.00 | 3A7 |
| ATOM | 2474 | O | LEU | 351 | 3.248 | −32.996 | 32.716 | 1.00 | 0.00 | 3A7 |
| ATOM | 2475 | N | GLN | 352 | 4.059 | −31.949 | 34.546 | 1.00 | 0.00 | 3A7 |
| ATOM | 2476 | CA | GLN | 352 | 2.775 | −31.491 | 35.037 | 1.00 | 0.00 | 3A7 |
| ATOM | 2477 | CB | GLN | 352 | 2.625 | −31.677 | 36.565 | 1.00 | 0.00 | 3A7 |
| ATOM | 2478 | CG | GLN | 352 | 2.109 | −33.072 | 36.977 | 1.00 | 0.00 | 3A7 |
| ATOM | 2479 | CD | GLN | 352 | 3.125 | −34.176 | 36.678 | 1.00 | 0.00 | 3A7 |
| ATOM | 2480 | OE1 | GLN | 352 | 2.971 | −34.938 | 35.716 | 1.00 | 0.00 | 3A7 |
| ATOM | 2481 | NE2 | GLN | 352 | 4.173 | −34.268 | 37.551 | 1.00 | 0.00 | 3A7 |
| ATOM | 2482 | C | GLN | 352 | 2.540 | −30.043 | 34.711 | 1.00 | 0.00 | 3A7 |
| ATOM | 2483 | O | GLN | 352 | 1.702 | −29.394 | 35.333 | 1.00 | 0.00 | 3A7 |
| ATOM | 2484 | N | LEU | 353 | 3.254 | −29.494 | 33.712 | 1.00 | 0.00 | 3A7 |
| ATOM | 2485 | CA | LEU | 353 | 3.130 | −28.104 | 33.338 | 1.00 | 0.00 | 3A7 |
| ATOM | 2486 | CB | LEU | 353 | 4.489 | −27.396 | 33.371 | 1.00 | 0.00 | 3A7 |
| ATOM | 2487 | CG | LEU | 353 | 4.991 | −27.076 | 34.789 | 1.00 | 0.00 | 3A7 |
| ATOM | 2488 | CD1 | LEU | 353 | 6.496 | −26.787 | 34.712 | 1.00 | 0.00 | 3A7 |
| ATOM | 2489 | CD2 | LEU | 353 | 4.215 | −25.911 | 35.433 | 1.00 | 0.00 | 3A7 |
| ATOM | 2490 | C | LEU | 353 | 2.588 | −28.000 | 31.951 | 1.00 | 0.00 | 3A7 |
| ATOM | 2491 | O | LEU | 353 | 3.198 | −27.396 | 31.071 | 1.00 | 0.00 | 3A7 |
| ATOM | 2492 | N | GLU | 354 | 1.380 | −28.556 | 31.747 | 1.00 | 0.00 | 3A7 |
| ATOM | 2493 | CA | GLU | 354 | 0.702 | −28.679 | 30.481 | 1.00 | 0.00 | 3A7 |
| ATOM | 2494 | CB | GLU | 354 | −0.697 | −29.284 | 30.692 | 1.00 | 0.00 | 3A7 |
| ATOM | 2495 | CG | GLU | 354 | −0.745 | −30.790 | 31.013 | 1.00 | 0.00 | 3A7 |
| ATOM | 2496 | CD | GLU | 354 | −0.273 | −31.081 | 32.431 | 1.00 | 0.00 | 3A7 |
| ATOM | 2497 | OE1 | GLU | 354 | 0.614 | −31.962 | 32.579 | 1.00 | 0.00 | 3A7 |
| ATOM | 2498 | OE2 | GLU | 354 | −0.801 | −30.446 | 33.382 | 1.00 | 0.00 | 3A7 |
| ATOM | 2499 | C | GLU | 354 | 0.533 | −27.384 | 29.710 | 1.00 | 0.00 | 3A7 |
| ATOM | 2500 | O | GLU | 354 | 0.869 | −27.315 | 28.531 | 1.00 | 0.00 | 3A7 |
| ATOM | 2501 | N | TYR | 355 | 0.017 | −26.319 | 30.358 | 1.00 | 0.00 | 3A7 |
| ATOM | 2502 | CA | TYR | 355 | −0.255 | −25.056 | 29.708 | 1.00 | 0.00 | 3A7 |
| ATOM | 2503 | CB | TYR | 355 | −1.107 | −24.126 | 30.584 | 1.00 | 0.00 | 3A7 |
| ATOM | 2504 | CG | TYR | 355 | −2.413 | −24.797 | 30.922 | 1.00 | 0.00 | 3A7 |
| ATOM | 2505 | CD1 | TYR | 355 | −2.853 | −24.843 | 32.245 | 1.00 | 0.00 | 3A7 |
| ATOM | 2506 | CD2 | TYR | 355 | −3.183 | −25.427 | 29.938 | 1.00 | 0.00 | 3A7 |
| ATOM | 2507 | CE1 | TYR | 355 | −4.029 | −25.504 | 32.585 | 1.00 | 0.00 | 3A7 |
| ATOM | 2508 | CE2 | TYR | 355 | −4.354 | −26.101 | 30.275 | 1.00 | 0.00 | 3A7 |
| ATOM | 2509 | CZ | TYR | 355 | −4.779 | −26.138 | 31.599 | 1.00 | 0.00 | 3A7 |
| ATOM | 2510 | OH | TYR | 355 | −5.972 | −26.813 | 31.940 | 1.00 | 0.00 | 3A7 |
| ATOM | 2511 | C | TYR | 355 | 1.014 | −24.314 | 29.358 | 1.00 | 0.00 | 3A7 |
| ATOM | 2512 | O | TYR | 355 | 1.093 | −23.672 | 28.320 | 1.00 | 0.00 | 3A7 |
| ATOM | 2513 | N | LEU | 356 | 2.069 | −24.432 | 30.194 | 1.00 | 0.00 | 3A7 |
| ATOM | 2514 | CA | LEU | 356 | 3.372 | −23.853 | 29.929 | 1.00 | 0.00 | 3A7 |
| ATOM | 2515 | CB | LEU | 356 | 4.307 | −24.000 | 31.136 | 1.00 | 0.00 | 3A7 |
| ATOM | 2516 | CG | LEU | 356 | 5.331 | −22.859 | 31.211 | 1.00 | 0.00 | 3A7 |
| ATOM | 2517 | CD1 | LEU | 356 | 5.499 | −22.429 | 32.674 | 1.00 | 0.00 | 3A7 |
| ATOM | 2518 | CD2 | LEU | 356 | 6.699 | −23.187 | 30.588 | 1.00 | 0.00 | 3A7 |
| ATOM | 2519 | C | LEU | 356 | 4.082 | −24.430 | 28.735 | 1.00 | 0.00 | 3A7 |
| ATOM | 2520 | O | LEU | 356 | 4.610 | −23.709 | 27.891 | 1.00 | 0.00 | 3A7 |
| ATOM | 2521 | N | ASP | 357 | 4.076 | −25.778 | 28.619 | 1.00 | 0.00 | 3A7 |
| ATOM | 2522 | CA | ASP | 357 | 4.652 | −26.516 | 27.513 | 1.00 | 0.00 | 3A7 |
| ATOM | 2523 | CB | ASP | 357 | 4.362 | −28.050 | 27.598 | 1.00 | 0.00 | 3A7 |
| ATOM | 2524 | CG | ASP | 357 | 5.259 | −28.771 | 28.597 | 1.00 | 0.00 | 3A7 |
| ATOM | 2525 | OD1 | ASP | 357 | 5.958 | −29.726 | 28.159 | 1.00 | 0.00 | 3A7 |
| ATOM | 2526 | OD2 | ASP | 357 | 5.258 | −28.397 | 29.796 | 1.00 | 0.00 | 3A7 |
| ATOM | 2527 | C | ASP | 357 | 4.055 | −26.136 | 26.182 | 1.00 | 0.00 | 3A7 |
| ATOM | 2528 | O | ASP | 357 | 4.748 | −25.985 | 25.183 | 1.00 | 0.00 | 3A7 |
| ATOM | 2529 | N | MET | 358 | 2.719 | −25.956 | 26.163 | 1.00 | 0.00 | 3A7 |
| ATOM | 2530 | CA | MET | 358 | 1.969 | −25.573 | 24.997 | 1.00 | 0.00 | 3A7 |
| ATOM | 2531 | CB | MET | 358 | 0.466 | −25.711 | 25.263 | 1.00 | 0.00 | 3A7 |
| ATOM | 2532 | CG | MET | 358 | 0.027 | −27.178 | 25.430 | 1.00 | 0.00 | 3A7 |
| ATOM | 2533 | SD | MET | 358 | −1.535 | −27.420 | 26.341 | 1.00 | 0.00 | 3A7 |
| ATOM | 2534 | CE | MET | 358 | −2.631 | −26.461 | 25.262 | 1.00 | 0.00 | 3A7 |
| ATOM | 2535 | C | MET | 358 | 2.269 | −24.149 | 24.601 | 1.00 | 0.00 | 3A7 |
| ATOM | 2536 | O | MET | 358 | 2.344 | −23.825 | 23.421 | 1.00 | 0.00 | 3A7 |
| ATOM | 2537 | N | VAL | 359 | 2.505 | −23.255 | 25.594 | 1.00 | 0.00 | 3A7 |
| ATOM | 2538 | CA | VAL | 359 | 2.885 | −21.869 | 25.376 | 1.00 | 0.00 | 3A7 |
| ATOM | 2539 | CB | VAL | 359 | 2.885 | −21.083 | 26.684 | 1.00 | 0.00 | 3A7 |
| ATOM | 2540 | CG1 | VAL | 359 | 3.710 | −19.776 | 26.626 | 1.00 | 0.00 | 3A7 |
| ATOM | 2541 | CG2 | VAL | 359 | 1.412 | −20.783 | 27.005 | 1.00 | 0.00 | 3A7 |
| ATOM | 2542 | C | VAL | 359 | 4.233 | −21.791 | 24.701 | 1.00 | 0.00 | 3A7 |
| ATOM | 2543 | O | VAL | 359 | 4.408 | −21.063 | 23.725 | 1.00 | 0.00 | 3A7 |
| ATOM | 2544 | N | VAL | 360 | 5.224 | −22.575 | 25.182 | 1.00 | 0.00 | 3A7 |
| ATOM | 2545 | CA | VAL | 360 | 6.556 | −22.600 | 24.616 | 1.00 | 0.00 | 3A7 |
| ATOM | 2546 | CB | VAL | 360 | 7.506 | −23.421 | 25.479 | 1.00 | 0.00 | 3A7 |
| ATOM | 2547 | CG1 | VAL | 360 | 8.897 | −23.573 | 24.831 | 1.00 | 0.00 | 3A7 |
| ATOM | 2548 | CG2 | VAL | 360 | 7.633 | −22.713 | 26.844 | 1.00 | 0.00 | 3A7 |
| ATOM | 2549 | C | VAL | 360 | 6.520 | −23.153 | 23.207 | 1.00 | 0.00 | 3A7 |
| ATOM | 2550 | O | VAL | 360 | 7.168 | −22.609 | 22.323 | 1.00 | 0.00 | 3A7 |
| ATOM | 2551 | N | ASN | 361 | 5.711 | −24.208 | 22.949 | 1.00 | 0.00 | 3A7 |
| ATOM | 2552 | CA | ASN | 361 | 5.556 | −24.789 | 21.630 | 1.00 | 0.00 | 3A7 |
| ATOM | 2553 | CB | ASN | 361 | 4.650 | −26.030 | 21.669 | 1.00 | 0.00 | 3A7 |
| ATOM | 2554 | CG | ASN | 361 | 5.417 | −27.219 | 22.258 | 1.00 | 0.00 | 3A7 |
| ATOM | 2555 | OD1 | ASH | 361 | 6.641 | −27.329 | 22.121 | 1.00 | 0.00 | 3A7 |
| ATOM | 2556 | ND2 | ASN | 361 | 4.654 | −28.135 | 22.930 | 1.00 | 0.00 | 3A7 |
| ATOM | 2557 | C | ASN | 361 | 4.952 | −23.808 | 20.655 | 1.00 | 0.00 | 3A7 |
| ATOM | 2558 | O | ASN | 361 | 5.410 | −23.694 | 19.523 | 1.00 | 0.00 | 3A7 |
| ATOM | 2559 | N | GLU | 362 | 3.925 | −23.044 | 21.087 | 1.00 | 0.00 | 3A7 |
| ATOM | 2560 | CA | GLU | 362 | 3.338 | −21.994 | 20.287 | 1.00 | 0.00 | 3A7 |
| ATOM | 2561 | CB | GLU | 362 | 2.013 | −21.499 | 20.896 | 1.00 | 0.00 | 3A7 |
| ATOM | 2562 | CG | GLU | 362 | 1.224 | −20.515 | 20.011 | 1.00 | 0.00 | 3A7 |
| ATOM | 2563 | CD | GLU | 362 | 0.583 | −21.183 | 18.798 | 1.00 | 0.00 | 3A7 |
| ATOM | 2564 | OE1 | GLU | 362 | 0.987 | −22.314 | 18.429 | 1.00 | 0.00 | 3A7 |
| ATOM | 2565 | OE2 | GLU | 362 | −0.309 | −20.534 | 18.190 | 1.00 | 0.00 | 3A7 |
| ATOM | 2566 | C | GLU | 362 | 4.241 | −20.837 | 20.000 | 1.00 | 0.00 | 3A7 |
| ATOM | 2567 | O | GLU | 362 | 4.205 | −20.266 | 18.913 | 1.00 | 0.00 | 3A7 |
| ATOM | 2568 | N | THR | 363 | 5.135 | −20.478 | 20.946 | 1.00 | 0.00 | 3A7 |
| ATOM | 2569 | CA | THR | 363 | 6.090 | −19.416 | 20.720 | 1.00 | 0.00 | 3A7 |
| ATOM | 2570 | CB | THR | 363 | 6.793 | −19.002 | 21.991 | 1.00 | 0.00 | 3A7 |
| ATOM | 2571 | OG1 | THR | 363 | 5.804 | −18.623 | 22.938 | 1.00 | 0.00 | 3A7 |
| ATOM | 2572 | CG2 | THR | 363 | 7.691 | −17.778 | 21.706 | 1.00 | 0.00 | 3A7 |
| ATOM | 2573 | C | THR | 363 | 7.091 | −19.831 | 19.666 | 1.00 | 0.00 | 3A7 |
| ATOM | 2574 | O | THR | 363 | 7.464 | −19.042 | 18.805 | 1.00 | 0.00 | 3A7 |
| ATOM | 2575 | N | LEU | 364 | 7.504 | −21.115 | 19.676 | 1.00 | 0.00 | 3A7 |
| ATOM | 2576 | CA | LEU | 364 | 8.417 | −21.641 | 18.695 | 1.00 | 0.00 | 3A7 |
| ATOM | 2577 | CB | LEU | 364 | 8.957 | −23.014 | 19.086 | 1.00 | 0.00 | 3A7 |
| ATOM | 2578 | CG | LEU | 364 | 9.921 | −23.018 | 20.283 | 1.00 | 0.00 | 3A7 |
| ATOM | 2579 | CD1 | LEU | 364 | 10.242 | −24.462 | 20.696 | 1.00 | 0.00 | 3A7 |
| ATOM | 2580 | CD2 | LEU | 364 | 11.219 | −22.259 | 19.974 | 1.00 | 0.00 | 3A7 |
| ATOM | 2581 | C | LEU | 364 | 7.769 | −21.746 | 17.338 | 1.00 | 0.00 | 3A7 |
| ATOM | 2582 | O | LEU | 364 | 8.410 | −21.492 | 16.324 | 1.00 | 0.00 | 3A7 |
| ATOM | 2583 | N | ARG | 365 | 6.467 | −22.094 | 17.264 | 1.00 | 0.00 | 3A7 |
| ATOM | 2584 | CA | ARG | 365 | 5.743 | −22.109 | 16.003 | 1.00 | 0.00 | 3A7 |
| ATOM | 2585 | CB | ARG | 365 | 4.300 | −22.612 | 16.169 | 1.00 | 0.00 | 3A7 |
| ATOM | 2586 | CG | ARG | 365 | 3.614 | −22.980 | 14.858 | 1.00 | 0.00 | 3A7 |
| ATOM | 2587 | CD | ARG | 365 | 2.117 | −23.285 | 15.005 | 1.00 | 0.00 | 3A7 |
| ATOM | 2588 | NE | ARG | 365 | 1.371 | −21.987 | 15.068 | 1.00 | 0.00 | 3A7 |
| ATOM | 2589 | CZ | ARG | 365 | 0.011 | −21.942 | 15.201 | 1.00 | 0.00 | 3A7 |
| ATOM | 2590 | NH1 | ARG | 365 | −0.637 | −20.742 | 15.124 | 1.00 | 0.00 | 3A7 |
| ATOM | 2591 | NH2 | ARG | 365 | −0.703 | −23.084 | 15.418 | 1.00 | 0.00 | 3A7 |
| ATOM | 2592 | C | ARG | 365 | 5.648 | −20.756 | 15.371 | 1.00 | 0.00 | 3A7 |
| ATOM | 2593 | O | ARG | 365 | 5.941 | −20.573 | 14.192 | 1.00 | 0.00 | 3A7 |
| ATOM | 2594 | N | LEU | 366 | 5.239 | −19.760 | 16.176 | 1.00 | 0.00 | 3A7 |
| ATOM | 2595 | CA | LEU | 366 | 5.030 | −18.410 | 15.729 | 1.00 | 0.00 | 3A7 |
| ATOM | 2596 | CB | LEU | 366 | 4.204 | −17.608 | 16.752 | 1.00 | 0.00 | 3A7 |
| ATOM | 2597 | CG | LEU | 366 | 3.017 | −16.833 | 16.122 | 1.00 | 0.00 | 3A7 |
| ATOM | 2598 | CD1 | LEU | 366 | 2.029 | −16.361 | 17.201 | 1.00 | 0.00 | 3A7 |
| ATOM | 2599 | CD2 | LEU | 366 | 3.452 | −15.648 | 15.240 | 1.00 | 0.00 | 3A7 |
| ATOM | 2600 | C | LEU | 366 | 6.295 | −17.670 | 15.380 | 1.00 | 0.00 | 3A7 |
| ATOM | 2601 | O | LEU | 366 | 6.339 | −16.992 | 14.359 | 1.00 | 0.00 | 3A7 |
| ATOM | 2602 | N | PHE | 367 | 7.367 | −17.798 | 16.193 | 1.00 | 0.00 | 3A7 |
| ATOM | 2603 | CA | PHE | 367 | 8.619 | −17.136 | 15.897 | 1.00 | 0.00 | 3A7 |
| ATOM | 2604 | CB | PHE | 367 | 8.999 | −16.072 | 16.907 | 1.00 | 0.00 | 3A7 |
| ATOM | 2605 | CG | PHE | 367 | 7.886 | −15.081 | 17.042 | 1.00 | 0.00 | 3A7 |
| ATOM | 2606 | CD1 | PHE | 367 | 6.921 | −15.250 | 18.035 | 1.00 | 0.00 | 3A7 |
| ATOM | 2607 | CD2 | PHE | 367 | 7.781 | −13.998 | 16.170 | 1.00 | 0.00 | 3A7 |
| ATOM | 2608 | CE1 | PHE | 367 | 5.855 | −14.365 | 18.137 | 1.00 | 0.00 | 3A7 |
| ATOM | 2609 | CE2 | PHE | 367 | 6.723 | −13.100 | 16.285 | 1.00 | 0.00 | 3A7 |
| ATOM | 2610 | CZ | PHE | 367 | 5.757 | −13.284 | 17.269 | 1.00 | 0.00 | 3A7 |
| ATOM | 2611 | C | PHE | 367 | 9.740 | −18.121 | 15.878 | 1.00 | 0.00 | 3A7 |
| ATOM | 2612 | O | PHE | 367 | 10.525 | −18.207 | 16.823 | 1.00 | 0.00 | 3A7 |
| ATOM | 2613 | N | PRO | 368 | 9.897 | −18.877 | 14.814 | 1.00 | 0.00 | 3A7 |
| ATOM | 2614 | CA | PRO | 368 | 10.926 | −19.868 | 14.730 | 1.00 | 0.00 | 3A7 |
| ATOM | 2615 | CD | PRO | 368 | 8.891 | −19.039 | 13.788 | 1.00 | 0.00 | 3A7 |
| ATOM | 2616 | CB | PRO | 368 | 10.570 | −20.710 | 13.544 | 1.00 | 0.00 | 3A7 |
| ATOM | 2617 | CG | PRO | 368 | 9.531 | −19.942 | 12.757 | 1.00 | 0.00 | 3A7 |
| ATOM | 2618 | C | PRO | 368 | 12.257 | −19.200 | 14.589 | 1.00 | 0.00 | 3A7 |
| ATOM | 2619 | O | PRO | 368 | 12.479 | −18.434 | 13.657 | 1.00 | 0.00 | 3A7 |
| ATOM | 2620 | N | VAL | 369 | 13.143 | −19.481 | 15.561 | 1.00 | 0.00 | 3A7 |
| ATOM | 2621 | CA | VAL | 369 | 14.427 | −18.858 | 15.709 | 1.00 | 0.00 | 3A7 |
| ATOM | 2622 | CB | VAL | 369 | 15.058 | −19.329 | 16.991 | 1.00 | 0.00 | 3A7 |
| ATOM | 2623 | CG1 | VAL | 369 | 16.449 | −18.705 | 17.115 | 1.00 | 0.00 | 3A7 |
| ATOM | 2624 | CG2 | VAL | 369 | 14.135 | −18.876 | 18.133 | 1.00 | 0.00 | 3A7 |
| ATOM | 2625 | C | VAL | 369 | 15.330 | −19.141 | 14.529 | 1.00 | 0.00 | 3A7 |
| ATOM | 2626 | O | VAL | 369 | 16.105 | −18.284 | 14.109 | 1.00 | 0.00 | 3A7 |
| ATOM | 2627 | N | ALA | 370 | 15.232 | −20.349 | 13.946 | 1.00 | 0.00 | 3A7 |
| ATOM | 2628 | CA | ALA | 370 | 15.984 | −20.686 | 12.775 | 1.00 | 0.00 | 3A7 |
| ATOM | 2629 | CB | ALA | 370 | 16.677 | −22.035 | 12.966 | 1.00 | 0.00 | 3A7 |
| ATOM | 2630 | C | ALA | 370 | 15.038 | −20.739 | 11.619 | 1.00 | 0.00 | 3A7 |
| ATOM | 2631 | O | ALA | 370 | 14.503 | −21.792 | 11.309 | 1.00 | 0.00 | 3A7 |
| ATOM | 2632 | N | MET | 371 | 14.813 | −19.597 | 10.938 | 1.00 | 0.00 | 3A7 |
| ATOM | 2633 | CA | MET | 371 | 13.787 | −19.436 | 9.924 | 1.00 | 0.00 | 3A7 |
| ATOM | 2634 | CB | MET | 371 | 13.599 | −17.940 | 9.616 | 1.00 | 0.00 | 3A7 |
| ATOM | 2635 | CG | MET | 371 | 14.852 | −17.053 | 9.800 | 1.00 | 0.00 | 3A7 |
| ATOM | 2636 | SD | MET | 371 | 16.175 | −17.276 | 8.566 | 1.00 | 0.00 | 3A7 |
| ATOM | 2637 | CE | MET | 371 | 15.303 | −16.484 | 7.182 | 1.00 | 0.00 | 3A7 |
| ATOM | 2638 | C | MET | 371 | 14.093 | −20.142 | 8.625 | 1.00 | 0.00 | 3A7 |
| ATOM | 2639 | O | MET | 371 | 13.208 | −20.260 | 7.783 | 1.00 | 0.00 | 3A7 |
| ATOM | 2640 | N | ARG | 372 | 15.346 | −20.621 | 8.432 | 1.00 | 0.00 | 3A7 |
| ATOM | 2641 | CA | ARG | 372 | 15.716 | −21.368 | 7.254 | 1.00 | 0.00 | 3A7 |
| ATOM | 2642 | CB | ARG | 372 | 16.208 | −20.480 | 6.088 | 1.00 | 0.00 | 3A7 |
| ATOM | 2643 | CG | ARG | 372 | 15.140 | −19.769 | 5.248 | 1.00 | 0.00 | 3A7 |
| ATOM | 2644 | CD | ARG | 372 | 15.828 | −19.020 | 4.096 | 1.00 | 0.00 | 3A7 |
| ATOM | 2645 | NE | ARG | 372 | 14.871 | −18.673 | 2.996 | 1.00 | 0.00 | 3A7 |
| ATOM | 2646 | CZ | ARG | 372 | 14.486 | −19.599 | 2.066 | 1.00 | 0.00 | 3A7 |
| ATOM | 2647 | NH1 | ARG | 372 | 13.971 | −19.194 | 0.870 | 1.00 | 0.00 | 3A7 |
| ATOM | 2648 | NH2 | ARG | 372 | 14.646 | −20.930 | 2.301 | 1.00 | 0.00 | 3A7 |
| ATOM | 2649 | C | ARG | 372 | 16.832 | −22.309 | 7.639 | 1.00 | 0.00 | 3A7 |
| ATOM | 2650 | O | ARG | 372 | 17.619 | −22.029 | 8.542 | 1.00 | 0.00 | 3A7 |
| ATOM | 2651 | N | LEU | 373 | 16.916 | −23.453 | 6.907 | 1.00 | 0.00 | 3A7 |
| ATOM | 2652 | CA | LEU | 373 | 17.947 | −24.462 | 7.034 | 1.00 | 0.00 | 3A7 |
| ATOM | 2653 | CB | LEU | 373 | 17.400 | −25.879 | 7.360 | 1.00 | 0.00 | 3A7 |
| ATOM | 2654 | CG | LEU | 373 | 16.633 | −26.016 | 8.700 | 1.00 | 0.00 | 3A7 |
| ATOM | 2655 | CD1 | LEU | 373 | 17.347 | −25.305 | 9.860 | 1.00 | 0.00 | 3A7 |
| ATOM | 2656 | CD2 | LEU | 373 | 15.150 | −25.605 | 8.611 | 1.00 | 0.00 | 3A7 |
| ATOM | 2657 | C | LEU | 373 | 18.641 | −24.532 | 5.697 | 1.00 | 0.00 | 3A7 |
| ATOM | 2658 | O | LEU | 373 | 18.145 | −23.979 | 4.717 | 1.00 | 0.00 | 3A7 |
| ATOM | 2659 | N | GLU | 374 | 19.806 | −25.232 | 5.619 | 1.00 | 0.00 | 3A7 |
| ATOM | 2660 | CA | GLU | 374 | 20.496 | −25.477 | 4.356 | 1.00 | 0.00 | 3A7 |
| ATOM | 2661 | CB | GLU | 374 | 21.788 | −24.637 | 4.153 | 1.00 | 0.00 | 3A7 |
| ATOM | 2662 | CG | GLU | 374 | 21.575 | −23.121 | 4.279 | 1.00 | 0.00 | 3A7 |
| ATOM | 2663 | CD | GLU | 374 | 22.909 | −22.422 | 4.038 | 1.00 | 0.00 | 3A7 |
| ATOM | 2664 | OE1 | GLU | 374 | 23.435 | −21.801 | 5.000 | 1.00 | 0.00 | 3A7 |
| ATOM | 2665 | OE2 | GLU | 374 | 23.420 | −22.501 | 2.889 | 1.00 | 0.00 | 3A7 |
| ATOM | 2666 | C | GLU | 374 | 20.862 | −26.936 | 4.222 | 1.00 | 0.00 | 3A7 |
| ATOM | 2667 | O | GLU | 374 | 21.393 | −27.553 | 5.141 | 1.00 | 0.00 | 3A7 |
| ATOM | 2668 | N | ARG | 375 | 20.591 | −27.510 | 3.026 | 1.00 | 0.00 | 3A7 |
| ATOM | 2669 | CA | ARG | 375 | 20.879 | −28.878 | 2.666 | 1.00 | 0.00 | 3A7 |
| ATOM | 2670 | CB | ARG | 375 | 19.614 | −29.770 | 2.636 | 1.00 | 0.00 | 3A7 |
| ATOM | 2671 | CG | ARG | 375 | 19.033 | −30.018 | 4.037 | 1.00 | 0.00 | 3A7 |
| ATOM | 2672 | CD | ARG | 375 | 17.789 | −30.919 | 4.035 | 1.00 | 0.00 | 3A7 |
| ATOM | 2673 | NE | ARG | 375 | 17.499 | −31.381 | 5.436 | 1.00 | 0.00 | 3A7 |
| ATOM | 2674 | CZ | ARG | 375 | 16.930 | −30.590 | 6.396 | 1.00 | 0.00 | 3A7 |
| ATOM | 2675 | NH1 | ARG | 375 | 16.734 | −31.096 | 7.650 | 1.00 | 0.00 | 3A7 |
| ATOM | 2676 | NH2 | ARG | 375 | 16.558 | −29.307 | 6.125 | 1.00 | 0.00 | 3A7 |
| ATOM | 2677 | C | ARG | 375 | 21.494 | −28.826 | 1.293 | 1.00 | 0.00 | 3A7 |
| ATOM | 2678 | O | ARG | 375 | 21.347 | −27.846 | 0.569 | 1.00 | 0.00 | 3A7 |
| ATOM | 2679 | N | VAL | 376 | 22.225 | −29.890 | 0.889 | 1.00 | 0.00 | 3A7 |
| ATOM | 2680 | CA | VAL | 376 | 22.836 | −29.949 | −0.420 | 1.00 | 0.00 | 3A7 |
| ATOM | 2681 | CB | VAL | 376 | 24.347 | −29.725 | −0.397 | 1.00 | 0.00 | 3A7 |
| ATOM | 2682 | CG1 | VAL | 376 | 24.936 | −29.796 | −1.822 | 1.00 | 0.00 | 3A7 |
| ATOM | 2683 | CG2 | VAL | 376 | 24.638 | −28.346 | 0.234 | 1.00 | 0.00 | 3A7 |
| ATOM | 2684 | C | VAL | 376 | 22.508 | −31.316 | −0.934 | 1.00 | 0.00 | 3A7 |
| ATOM | 2685 | O | VAL | 376 | 22.653 | −32.316 | −0.233 | 1.00 | 0.00 | 3A7 |
| ATOM | 2686 | N | CYS | 377 | 22.071 | −31.397 | −2.212 | 1.00 | 0.00 | 3A7 |
| ATOM | 2687 | CA | CYS | 377 | 21.721 | −32.642 | −2.851 | 1.00 | 0.00 | 3A7 |
| ATOM | 2688 | CB | CYS | 377 | 20.821 | −32.435 | −4.083 | 1.00 | 0.00 | 3A7 |
| ATOM | 2689 | SG | CYS | 377 | 19.174 | −31.876 | −3.569 | 1.00 | 0.00 | 3A7 |
| ATOM | 2690 | C | CYS | 377 | 22.964 | −33.392 | −3.254 | 1.00 | 0.00 | 3A7 |
| ATOM | 2691 | O | CYS | 377 | 23.919 | −32.843 | −3.794 | 1.00 | 0.00 | 3A7 |
| ATOM | 2692 | N | LYS | 378 | 22.964 | −34.705 | −2.955 | 1.00 | 0.00 | 3A7 |
| ATOM | 2693 | CA | LYS | 378 | 24.083 | −35.594 | −3.143 | 1.00 | 0.00 | 3A7 |
| ATOM | 2694 | CB | LYS | 378 | 24.073 | −36.718 | −2.075 | 1.00 | 0.00 | 3A7 |
| ATOM | 2695 | CG | LYS | 378 | 23.127 | −36.428 | −0.894 | 1.00 | 0.00 | 3A7 |
| ATOM | 2696 | CD | LYS | 378 | 23.024 | −37.592 | 0.101 | 1.00 | 0.00 | 3A7 |
| ATOM | 2697 | CE | LYS | 378 | 21.948 | −37.375 | 1.174 | 1.00 | 0.00 | 3A7 |
| ATOM | 2698 | NZ | LYS | 378 | 22.223 | −36.159 | 1.974 | 1.00 | 0.00 | 3A7 |
| ATOM | 2699 | C | LYS | 378 | 24.040 | −36.202 | −4.524 | 1.00 | 0.00 | 3A7 |
| ATOM | 2700 | O | LYS | 378 | 25.062 | −36.609 | −5.075 | 1.00 | 0.00 | 3A7 |
| ATOM | 2701 | N | LYS | 379 | 22.814 | −36.285 | −5.081 | 1.00 | 0.00 | 3A7 |
| ATOM | 2702 | CA | LYS | 379 | 22.547 | −36.939 | −6.329 | 1.00 | 0.00 | 3A7 |
| ATOM | 2703 | CB | LYS | 379 | 22.121 | −38.417 | −6.127 | 1.00 | 0.00 | 3A7 |
| ATOM | 2704 | CG | LYS | 379 | 20.935 | −38.609 | −5.161 | 1.00 | 0.00 | 3A7 |
| ATOM | 2705 | CD | LYS | 379 | 20.582 | −40.083 | −4.899 | 1.00 | 0.00 | 3A7 |
| ATOM | 2706 | CE | LYS | 379 | 21.665 | −40.842 | −4.120 | 1.00 | 0.00 | 3A7 |
| ATOM | 2707 | NZ | LYS | 379 | 21.236 | −42.233 | −3.850 | 1.00 | 0.00 | 3A7 |
| ATOM | 2708 | C | LYS | 379 | 21.450 | −36.155 | −6.981 | 1.00 | 0.00 | 3A7 |
| ATOM | 2709 | O | LYS | 379 | 21.014 | −35.119 | −6.483 | 1.00 | 0.00 | 3A7 |
| ATOM | 2710 | N | ASP | 380 | 20.951 | −36.650 | −8.134 | 1.00 | 0.00 | 3A7 |
| ATOM | 2711 | CA | ASP | 380 | 19.820 | −36.071 | −8.818 | 1.00 | 0.00 | 3A7 |
| ATOM | 2712 | CB | ASP | 380 | 19.811 | −36.404 | −10.323 | 1.00 | 0.00 | 3A7 |
| ATOM | 2713 | CG | ASP | 380 | 21.092 | −35.921 | −10.999 | 1.00 | 0.00 | 3A7 |
| ATOM | 2714 | OD1 | ASP | 380 | 21.347 | −34.689 | −10.981 | 1.00 | 0.00 | 3A7 |
| ATOM | 2715 | OD2 | ASP | 380 | 21.824 | −36.782 | −11.556 | 1.00 | 0.00 | 3A7 |
| ATOM | 2716 | C | ASP | 380 | 18.562 | −36.616 | −8.182 | 1.00 | 0.00 | 3A7 |
| ATOM | 2717 | O | ASP | 380 | 18.496 | −37.798 | −7.848 | 1.00 | 0.00 | 3A7 |
| ATOM | 2718 | N | VAL | 381 | 17.554 | −35.744 | −7.966 | 1.00 | 0.00 | 3A7 |
| ATOM | 2719 | CA | VAL | 381 | 16.378 | −36.118 | −7.224 | 1.00 | 0.00 | 3A7 |
| ATOM | 2720 | CB | VAL | 381 | 16.557 | −35.910 | −5.720 | 1.00 | 0.00 | 3A7 |
| ATOM | 2721 | CG1 | VAL | 381 | 16.853 | −34.439 | −5.360 | 1.00 | 0.00 | 3A7 |
| ATOM | 2722 | CG2 | VAL | 381 | 15.353 | −36.489 | −4.947 | 1.00 | 0.00 | 3A7 |
| ATOM | 2723 | C | VAL | 381 | 15.248 | −35.308 | −7.781 | 1.00 | 0.00 | 3A7 |
| ATOM | 2724 | O | VAL | 381 | 15.417 | −34.151 | −8.142 | 1.00 | 0.00 | 3A7 |
| ATOM | 2725 | N | GLU | 382 | 14.045 | −35.908 | −7.874 | 1.00 | 0.00 | 3A7 |
| ATOM | 2726 | CA | GLU | 382 | 12.888 | −35.247 | −8.420 | 1.00 | 0.00 | 3A7 |
| ATOM | 2727 | CB | GLU | 382 | 12.165 | −36.100 | −9.485 | 1.00 | 0.00 | 3A7 |
| ATOM | 2728 | CG | GLU | 382 | 12.963 | −36.325 | −10.781 | 1.00 | 0.00 | 3A7 |
| ATOM | 2729 | CD | GLU | 382 | 14.109 | −37.311 | −10.571 | 1.00 | 0.00 | 3A7 |
| ATOM | 2730 | OE1 | GLU | 382 | 13.827 | −38.470 | −10.163 | 1.00 | 0.00 | 3A7 |
| ATOM | 2731 | OE2 | GLU | 382 | 15.281 | −36.921 | −10.822 | 1.00 | 0.00 | 3A7 |
| ATOM | 2732 | C | GLU | 382 | 11.952 | −34.980 | −7.271 | 1.00 | 0.00 | 3A7 |
| ATOM | 2733 | O | GLU | 382 | 11.531 | −35.909 | −6.587 | 1.00 | 0.00 | 3A7 |
| ATOM | 2734 | N | ILE | 383 | 11.615 | −33.690 | −7.028 | 1.00 | 0.00 | 3A7 |
| ATOM | 2735 | CA | ILE | 383 | 10.762 | −33.270 | −5.937 | 1.00 | 0.00 | 3A7 |
| ATOM | 2736 | CB | ILE | 383 | 11.449 | −32.299 | −4.973 | 1.00 | 0.00 | 3A7 |
| ATOM | 2737 | CG2 | ILE | 383 | 10.543 | −32.043 | −3.747 | 1.00 | 0.00 | 3A7 |
| ATOM | 2738 | CG1 | ILE | 383 | 12.865 | −32.774 | −4.555 | 1.00 | 0.00 | 3A7 |
| ATOM | 2739 | CD | ILE | 383 | 12.883 | −34.026 | −3.677 | 1.00 | 0.00 | 3A7 |
| ATOM | 2740 | C | ILE | 383 | 9.578 | −32.594 | −6.567 | 1.00 | 0.00 | 3A7 |
| ATOM | 2741 | O | ILE | 383 | 9.686 | −31.493 | −7.103 | 1.00 | 0.00 | 3A7 |
| ATOM | 2742 | N | ASN | 384 | 8.399 | −33.254 | −6.521 | 1.00 | 0.00 | 3A7 |
| ATOM | 2743 | CA | ASN | 384 | 7.130 | −32.719 | −6.976 | 1.00 | 0.00 | 3A7 |
| ATOM | 2744 | CB | ASN | 384 | 6.732 | −31.411 | −6.222 | 1.00 | 0.00 | 3A7 |
| ATOM | 2745 | CG | ASN | 384 | 5.218 | −31.147 | −6.283 | 1.00 | 0.00 | 3A7 |
| ATOM | 2746 | OD1 | ASN | 384 | 4.758 | −30.251 | −7.000 | 1.00 | 0.00 | 3A7 |
| ATOM | 2747 | ND2 | ASN | 384 | 4.443 | −31.956 | −5.498 | 1.00 | 0.00 | 3A7 |
| ATOM | 2748 | C | ASN | 384 | 7.100 | −32.504 | −8.480 | 1.00 | 0.00 | 3A7 |
| ATOM | 2749 | O | ASN | 384 | 6.410 | −31.623 | −8.988 | 1.00 | 0.00 | 3A7 |
| ATOM | 2750 | N | GLY | 385 | 7.870 | −33.322 | −9.231 | 1.00 | 0.00 | 3A7 |
| ATOM | 2751 | CA | GLY | 385 | 7.911 | −33.246 | −10.666 | 1.00 | 0.00 | 3A7 |
| ATOM | 2752 | C | GLY | 385 | 9.013 | −32.342 | −11.156 | 1.00 | 0.00 | 3A7 |
| ATOM | 2753 | O | GLY | 385 | 9.217 | −32.266 | −12.366 | 1.00 | 0.00 | 3A7 |
| ATOM | 2754 | N | MET | 386 | 9.762 | −31.631 | −10.264 | 1.00 | 0.00 | 3A7 |
| ATOM | 2755 | CA | MET | 386 | 10.855 | −30.773 | −10.695 | 1.00 | 0.00 | 3A7 |
| ATOM | 2756 | CB | MET | 386 | 10.660 | −29.264 | −10.426 | 1.00 | 0.00 | 3A7 |
| ATOM | 2757 | CG | MET | 386 | 10.337 | −28.845 | −8.990 | 1.00 | 0.00 | 3A7 |
| ATOM | 2758 | SD | MET | 386 | 9.854 | −27.091 | −8.895 | 1.00 | 0.00 | 3A7 |
| ATOM | 2759 | CE | MET | 386 | 11.448 | −26.373 | −9.388 | 1.00 | 0.00 | 3A7 |
| ATOM | 2760 | C | MET | 386 | 12.159 | −31.305 | −10.187 | 1.00 | 0.00 | 3A7 |
| ATOM | 2761 | O | MET | 386 | 12.335 | −31.654 | −9.025 | 1.00 | 0.00 | 3A7 |
| ATOM | 2762 | N | PHE | 387 | 13.122 | −31.396 | −11.125 | 1.00 | 0.00 | 3A7 |
| ATOM | 2763 | CA | PHE | 387 | 14.382 | −32.074 | −10.982 | 1.00 | 0.00 | 3A7 |
| ATOM | 2764 | CB | PHE | 387 | 14.935 | −32.474 | −12.378 | 1.00 | 0.00 | 3A7 |
| ATOM | 2765 | CG | PHE | 387 | 14.102 | −33.505 | −13.091 | 1.00 | 0.00 | 3A7 |
| ATOM | 2766 | CD1 | PHE | 387 | 14.645 | −34.767 | −13.344 | 1.00 | 0.00 | 3A7 |
| ATOM | 2767 | CD2 | PHE | 387 | 12.827 | −33.213 | −13.585 | 1.00 | 0.00 | 3A7 |
| ATOM | 2768 | CE1 | PHE | 387 | 13.920 | −35.727 | −14.045 | 1.00 | 0.00 | 3A7 |
| ATOM | 2769 | CE2 | PHE | 387 | 12.097 | −34.173 | −14.280 | 1.00 | 0.00 | 3A7 |
| ATOM | 2770 | CZ | PHE | 387 | 12.642 | −35.433 | −14.507 | 1.00 | 0.00 | 3A7 |
| ATOM | 2771 | C | PHE | 387 | 15.405 | −31.180 | −10.312 | 1.00 | 0.00 | 3A7 |
| ATOM | 2772 | O | PHE | 387 | 15.631 | −30.043 | −10.726 | 1.00 | 0.00 | 3A7 |
| ATOM | 2773 | N | ILE | 388 | 16.066 | −31.706 | −9.251 | 1.00 | 0.00 | 3A7 |
| ATOM | 2774 | CA | ILE | 388 | 17.145 | −31.049 | −8.544 | 1.00 | 0.00 | 3A7 |
| ATOM | 2775 | CB | ILE | 388 | 16.994 | −31.071 | −7.032 | 1.00 | 0.00 | 3A7 |
| ATOM | 2776 | CG2 | ILE | 388 | 18.144 | −30.276 | −6.387 | 1.00 | 0.00 | 3A7 |
| ATOM | 2777 | CG1 | ILE | 388 | 15.641 | −30.487 | −6.591 | 1.00 | 0.00 | 3A7 |
| ATOM | 2778 | CD | ILE | 388 | 15.434 | −29.010 | −6.913 | 1.00 | 0.00 | 3A7 |
| ATOM | 2779 | C | ILE | 388 | 18.438 | −31.746 | −8.935 | 1.00 | 0.00 | 3A7 |
| ATOM | 2780 | O | ILE | 388 | 18.576 | −32.952 | −8.728 | 1.00 | 0.00 | 3A7 |
| ATOM | 2781 | N | PRO | 389 | 19.415 | −31.021 | −9.496 | 1.00 | 0.00 | 3A7 |
| ATOM | 2782 | CA | PRO | 389 | 20.698 | −31.581 | −9.867 | 1.00 | 0.00 | 3A7 |
| ATOM | 2783 | CD | PRO | 389 | 19.063 | −29.901 | −10.375 | 1.00 | 0.00 | 3A7 |
| ATOM | 2784 | CB | PRO | 389 | 21.290 | −30.549 | −10.822 | 1.00 | 0.00 | 3A7 |
| ATOM | 2785 | CG | PRO | 389 | 20.081 | −29.931 | −11.509 | 1.00 | 0.00 | 3A7 |
| ATOM | 2786 | C | PRO | 389 | 21.614 | −31.785 | −8.684 | 1.00 | 0.00 | 3A7 |
| ATOM | 2787 | O | PRO | 389 | 21.474 | −31.121 | −7.659 | 1.00 | 0.00 | 3A7 |
| ATOM | 2788 | N | LYS | 390 | 22.627 | −32.663 | −8.836 | 1.00 | 0.00 | 3A7 |
| ATOM | 2789 | CA | LYS | 390 | 23.654 | −32.884 | −7.847 | 1.00 | 0.00 | 3A7 |
| ATOM | 2790 | CB | LYS | 390 | 24.733 | −33.851 | −8.363 | 1.00 | 0.00 | 3A7 |
| ATOM | 2791 | CG | LYS | 390 | 24.234 | −35.053 | −9.176 | 1.00 | 0.00 | 3A7 |
| ATOM | 2792 | CD | LYS | 390 | 25.372 | −36.029 | −9.517 | 1.00 | 0.00 | 3A7 |
| ATOM | 2793 | CE | LYS | 390 | 24.921 | −37.258 | −10.316 | 1.00 | 0.00 | 3A7 |
| ATOM | 2794 | NZ | LYS | 390 | 23.959 | −38.073 | −9.538 | 1.00 | 0.00 | 3A7 |
| ATOM | 2795 | C | LYS | 390 | 24.346 | −31.584 | −7.486 | 1.00 | 0.00 | 3A7 |
| ATOM | 2796 | O | LYS | 390 | 24.631 | −30.776 | −8.366 | 1.00 | 0.00 | 3A7 |
| ATOM | 2797 | N | GLY | 391 | 24.558 | −31.329 | −6.177 | 1.00 | 0.00 | 3A7 |
| ATOM | 2798 | CA | GLY | 391 | 25.250 | −30.153 | −5.707 | 1.00 | 0.00 | 3A7 |
| ATOM | 2799 | C | GLY | 391 | 24.397 | −28.926 | −5.567 | 1.00 | 0.00 | 3A7 |
| ATOM | 2800 | O | GLY | 391 | 24.924 | −27.848 | −5.320 | 1.00 | 0.00 | 3A7 |
| ATOM | 2801 | N | VAL | 392 | 23.052 | −29.029 | −5.690 | 1.00 | 0.00 | 3A7 |
| ATOM | 2802 | CA | VAL | 392 | 22.159 | −27.896 | −5.522 | 1.00 | 0.00 | 3A7 |
| ATOM | 2803 | CB | VAL | 392 | 20.829 | −28.126 | −6.224 | 1.00 | 0.00 | 3A7 |
| ATOM | 2804 | CG1 | VAL | 392 | 19.765 | −27.062 | −5.883 | 1.00 | 0.00 | 3A7 |
| ATOM | 2805 | CG2 | VAL | 392 | 21.074 | −28.134 | −7.744 | 1.00 | 0.00 | 3A7 |
| ATOM | 2806 | C | VAL | 392 | 21.922 | −27.684 | −4.050 | 1.00 | 0.00 | 3A7 |
| ATOM | 2807 | O | VAL | 392 | 21.849 | −28.644 | −3.290 | 1.00 | 0.00 | 3A7 |
| ATOM | 2808 | N | VAL | 393 | 21.787 | −26.403 | −3.629 | 1.00 | 0.00 | 3A7 |
| ATOM | 2809 | CA | VAL | 393 | 21.491 | −26.024 | −2.268 | 1.00 | 0.00 | 3A7 |
| ATOM | 2810 | CB | VAL | 393 | 22.118 | −24.695 | −1.900 | 1.00 | 0.00 | 3A7 |
| ATOM | 2811 | CG1 | VAL | 393 | 21.828 | −24.336 | −0.425 | 1.00 | 0.00 | 3A7 |
| ATOM | 2812 | CG2 | VAL | 393 | 23.636 | −24.785 | −2.160 | 1.00 | 0.00 | 3A7 |
| ATOM | 2813 | C | VAL | 393 | 19.992 | −25.964 | −2.130 | 1.00 | 0.00 | 3A7 |
| ATOM | 2814 | O | VAL | 393 | 19.308 | −25.288 | −2.891 | 1.00 | 0.00 | 3A7 |
| ATOM | 2815 | N | VAL | 394 | 19.457 | −26.712 | −1.145 | 1.00 | 0.00 | 3A7 |
| ATOM | 2816 | CA | VAL | 394 | 18.049 | −26.825 | −0.864 | 1.00 | 0.00 | 3A7 |
| ATOM | 2817 | CB | VAL | 394 | 17.609 | −28.282 | −0.810 | 1.00 | 0.00 | 3A7 |
| ATOM | 2818 | CG1 | VAL | 394 | 16.104 | −28.411 | −0.510 | 1.00 | 0.00 | 3A7 |
| ATOM | 2819 | CG2 | VAL | 394 | 17.948 | −28.936 | −2.163 | 1.00 | 0.00 | 3A7 |
| ATOM | 2820 | C | VAL | 394 | 17.840 | −26.177 | 0.471 | 1.00 | 0.00 | 3A7 |
| ATOM | 2821 | O | VAL | 394 | 18.601 | −26.423 | 1.400 | 1.00 | 0.00 | 3A7 |
| ATOM | 2822 | N | MET | 395 | 16.800 | −25.325 | 0.609 | 1.00 | 0.00 | 3A7 |
| ATOM | 2823 | CA | MET | 395 | 16.529 | −24.656 | 1.856 | 1.00 | 0.00 | 3A7 |
| ATOM | 2824 | CB | MET | 395 | 16.818 | −23.145 | 1.809 | 1.00 | 0.00 | 3A7 |
| ATOM | 2825 | CG | MET | 395 | 18.304 | −22.812 | 1.578 | 1.00 | 0.00 | 3A7 |
| ATOM | 2826 | SD | MET | 395 | 18.747 | −21.101 | 2.015 | 1.00 | 0.00 | 3A7 |
| ATOM | 2827 | CE | MET | 395 | 17.770 | −20.277 | 0.725 | 1.00 | 0.00 | 3A7 |
| ATOM | 2828 | C | MET | 395 | 15.093 | −24.885 | 2.216 | 1.00 | 0.00 | 3A7 |
| ATOM | 2829 | O | MET | 395 | 14.195 | −24.925 | 1.389 | 1.00 | 0.00 | 3A7 |
| ATOM | 2830 | N | ILE | 396 | 14.842 | −25.076 | 3.515 | 1.00 | 0.00 | 3A7 |
| ATOM | 2831 | CA | ILE | 396 | 13.536 | −25.356 | 4.059 | 1.00 | 0.00 | 3A7 |
| ATOM | 2832 | CB | ILE | 396 | 13.599 | −26.610 | 4.904 | 1.00 | 0.00 | 3A7 |
| ATOM | 2833 | CG2 | ILE | 396 | 12.490 | −26.667 | 5.983 | 1.00 | 0.00 | 3A7 |
| ATOM | 2834 | CG1 | ILE | 396 | 13.729 | −27.897 | 4.057 | 1.00 | 0.00 | 3A7 |
| ATOM | 2835 | CD | ILE | 396 | 15.087 | −28.104 | 3.373 | 1.00 | 0.00 | 3A7 |
| ATOM | 2836 | C | ILE | 396 | 13.188 | −24.182 | 4.902 | 1.00 | 0.00 | 3A7 |
| ATOM | 2837 | O | ILE | 396 | 13.874 | −23.964 | 5.894 | 1.00 | 0.00 | 3A7 |
| ATOM | 2838 | N | PRO | 397 | 12.167 | −23.398 | 4.590 | 1.00 | 0.00 | 3A7 |
| ATOM | 2839 | CA | PRO | 397 | 11.790 | −22.297 | 5.462 | 1.00 | 0.00 | 3A7 |
| ATOM | 2840 | CD | PRO | 397 | 11.961 | −23.022 | 3.209 | 1.00 | 0.00 | 3A7 |
| ATOM | 2841 | CB | PRO | 397 | 11.201 | −21.252 | 4.533 | 1.00 | 0.00 | 3A7 |
| ATOM | 2842 | CG | PRO | 397 | 10.809 | −22.043 | 3.293 | 1.00 | 0.00 | 3A7 |
| ATOM | 2843 | C | PRO | 397 | 10.834 | −22.717 | 6.544 | 1.00 | 0.00 | 3A7 |
| ATOM | 2844 | O | PRO | 397 | 9.629 | −22.732 | 6.310 | 1.00 | 0.00 | 3A7 |
| ATOM | 2845 | N | SER | 398 | 11.352 | −22.981 | 7.767 | 1.00 | 0.00 | 3A7 |
| ATOM | 2846 | CA | SER | 398 | 10.575 | −23.392 | 8.914 | 1.00 | 0.00 | 3A7 |
| ATOM | 2847 | CB | SER | 398 | 11.471 | −23.676 | 10.138 | 1.00 | 0.00 | 3A7 |
| ATOM | 2848 | OG | SER | 398 | 12.135 | −22.517 | 10.613 | 1.00 | 0.00 | 3A7 |
| ATOM | 2849 | C | SER | 398 | 9.531 | −22.379 | 9.316 | 1.00 | 0.00 | 3A7 |
| ATOM | 2850 | O | SER | 398 | 8.422 | −22.745 | 9.668 | 1.00 | 0.00 | 3A7 |
| ATOM | 2851 | N | TYR | 399 | 9.838 | −21.053 | 9.231 | 1.00 | 0.00 | 3A7 |
| ATOM | 2852 | CA | TYR | 399 | 8.913 | −19.958 | 9.507 | 1.00 | 0.00 | 3A7 |
| ATOM | 2853 | CB | TYR | 399 | 9.578 | −18.562 | 9.264 | 1.00 | 0.00 | 3A7 |
| ATOM | 2854 | CG | TYR | 399 | 9.084 | −17.492 | 10.212 | 1.00 | 0.00 | 3A7 |
| ATOM | 2855 | CD1 | TYR | 399 | 9.933 | −16.936 | 11.172 | 1.00 | 0.00 | 3A7 |
| ATOM | 2856 | CD2 | TYR | 399 | 7.775 | −17.008 | 10.123 | 1.00 | 0.00 | 3A7 |
| ATOM | 2857 | CE1 | TYR | 399 | 9.473 | −15.959 | 12.051 | 1.00 | 0.00 | 3A7 |
| ATOM | 2858 | CE2 | TYR | 399 | 7.308 | −16.041 | 11.007 | 1.00 | 0.00 | 3A7 |
| ATOM | 2859 | CZ | TYR | 399 | 8.156 | −15.520 | 11.975 | 1.00 | 0.00 | 3A7 |
| ATOM | 2860 | OH | TYR | 399 | 7.673 | −14.553 | 12.880 | 1.00 | 0.00 | 3A7 |
| ATOM | 2861 | C | TYR | 399 | 7.688 | −20.038 | 8.647 | 1.00 | 0.00 | 3A7 |
| ATOM | 2862 | O | TYR | 399 | 6.567 | −19.765 | 9.067 | 1.00 | 0.00 | 3A7 |
| ATOM | 2863 | N | VAL | 400 | 7.911 | −20.472 | 7.398 | 1.00 | 0.00 | 3A7 |
| ATOM | 2864 | CA | VAL | 400 | 6.877 | −20.616 | 6.436 | 1.00 | 0.00 | 3A7 |
| ATOM | 2865 | CB | VAL | 400 | 7.444 | −20.658 | 5.045 | 1.00 | 0.00 | 3A7 |
| ATOM | 2866 | CG1 | VAL | 400 | 6.294 | −20.758 | 4.020 | 1.00 | 0.00 | 3A7 |
| ATOM | 2867 | CG2 | VAL | 400 | 8.183 | −19.308 | 4.899 | 1.00 | 0.00 | 3A7 |
| ATOM | 2868 | C | VAL | 400 | 6.002 | −21.827 | 6.653 | 1.00 | 0.00 | 3A7 |
| ATOM | 2869 | O | VAL | 400 | 4.781 | −21.734 | 6.547 | 1.00 | 0.00 | 3A7 |
| ATOM | 2870 | N | LEU | 401 | 6.602 | −22.992 | 6.970 | 1.00 | 0.00 | 3A7 |
| ATOM | 2871 | CA | LEU | 401 | 5.875 | −24.224 | 7.175 | 1.00 | 0.00 | 3A7 |
| ATOM | 2872 | CB | LEU | 401 | 6.790 | −25.449 | 7.318 | 1.00 | 0.00 | 3A7 |
| ATOM | 2873 | CG | LEU | 401 | 8.101 | −25.453 | 6.535 | 1.00 | 0.00 | 3A7 |
| ATOM | 2874 | CD1 | LEU | 401 | 8.877 | −26.722 | 6.915 | 1.00 | 0.00 | 3A7 |
| ATOM | 2875 | CD2 | LEU | 401 | 7.904 | −25.294 | 5.023 | 1.00 | 0.00 | 3A7 |
| ATOM | 2876 | C | LEU | 401 | 5.031 | −24.186 | 8.449 | 1.00 | 0.00 | 3A7 |
| ATOM | 2877 | O | LEU | 401 | 3.918 | −24.705 | 8.504 | 1.00 | 0.00 | 3A7 |
| ATOM | 2878 | N | HIS | 402 | 5.545 | −23.512 | 9.509 | 1.00 | 0.00 | 3A7 |
| ATOM | 2879 | CA | HIS | 402 | 4.879 | −23.303 | 10.782 | 1.00 | 0.00 | 3A7 |
| ATOM | 2880 | ND1 | HIS | 402 | 7.090 | −24.809 | 12.042 | 1.00 | 0.00 | 3A7 |
| ATOM | 2881 | CG | HIS | 402 | 6.758 | −23.553 | 12.480 | 1.00 | 0.00 | 3A7 |
| ATOM | 2882 | CB | HIS | 402 | 5.843 | −22.628 | 11.778 | 1.00 | 0.00 | 3A7 |
| ATOM | 2883 | NE2 | HIS | 402 | 8.734 | −24.122 | 13.375 | 1.00 | 0.00 | 3A7 |
| ATOM | 2884 | CD2 | HIS | 402 | 7.762 | −23.155 | 13.305 | 1.00 | 0.00 | 3A7 |
| ATOM | 2885 | CE1 | HIS | 402 | 8.289 | −25.085 | 12.595 | 1.00 | 0.00 | 3A7 |
| ATOM | 2886 | C | HIS | 402 | 3.648 | −22.420 | 10.674 | 1.00 | 0.00 | 3A7 |
| ATOM | 2887 | O | HIS | 402 | 2.844 | −22.343 | 11.602 | 1.00 | 0.00 | 3A7 |
| ATOM | 2888 | N | HIS | 403 | 3.485 | −21.730 | 9.522 | 1.00 | 0.00 | 3A7 |
| ATOM | 2889 | CA | HIS | 403 | 2.382 | −20.839 | 9.280 | 1.00 | 0.00 | 3A7 |
| ATOM | 2890 | ND1 | HIS | 403 | 4.537 | −19.115 | 10.966 | 1.00 | 0.00 | 3A7 |
| ATOM | 2891 | CG | HIS | 403 | 3.389 | −18.746 | 10.299 | 1.00 | 0.00 | 3A7 |
| ATOM | 2892 | CB | HIS | 403 | 2.858 | −19.394 | 9.048 | 1.00 | 0.00 | 3A7 |
| ATOM | 2893 | NE2 | HIS | 403 | 3.638 | −17.451 | 12.131 | 1.00 | 0.00 | 3A7 |
| ATOM | 2894 | CD2 | HIS | 403 | 2.852 | −17.730 | 11.028 | 1.00 | 0.00 | 3A7 |
| ATOM | 2895 | CE1 | HIS | 403 | 4.638 | −18.308 | 12.051 | 1.00 | 0.00 | 3A7 |
| ATOM | 2896 | C | HIS | 403 | 1.564 | −21.280 | 8.096 | 1.00 | 0.00 | 3A7 |
| ATOM | 2897 | O | HIS | 403 | 0.830 | −20.492 | 7.500 | 1.00 | 0.00 | 3A7 |
| ATOM | 2898 | N | ASP | 404 | 1.605 | −22.588 | 7.740 | 1.00 | 0.00 | 3A7 |
| ATOM | 2899 | CA | ASP | 404 | 0.811 | −23.143 | 6.662 | 1.00 | 0.00 | 3A7 |
| ATOM | 2900 | CB | ASP | 404 | 1.274 | −24.585 | 6.322 | 1.00 | 0.00 | 3A7 |
| ATOM | 2901 | CG | ASP | 404 | 0.584 | −25.218 | 5.103 | 1.00 | 0.00 | 3A7 |
| ATOM | 2902 | OD1 | ASP | 404 | −0.209 | −24.540 | 4.402 | 1.00 | 0.00 | 3A7 |
| ATOM | 2903 | OD2 | ASP | 404 | 0.879 | −26.417 | 4.850 | 1.00 | 0.00 | 3A7 |
| ATOM | 2904 | C | ASP | 404 | −0.645 | −23.164 | 7.105 | 1.00 | 0.00 | 3A7 |
| ATOM | 2905 | O | ASP | 404 | −0.941 | −23.809 | 8.108 | 1.00 | 0.00 | 3A7 |
| ATOM | 2906 | N | PRO | 405 | −1.568 | −22.474 | 6.421 | 1.00 | 0.00 | 3A7 |
| ATOM | 2907 | CA | PRO | 405 | −2.958 | −22.344 | 6.827 | 1.00 | 0.00 | 3A7 |
| ATOM | 2908 | CD | PRO | 405 | −1.277 | −21.674 | 5.237 | 1.00 | 0.00 | 3A7 |
| ATOM | 2909 | CB | PRO | 405 | −3.543 | −21.309 | 5.854 | 1.00 | 0.00 | 3A7 |
| ATOM | 2910 | CG | PRO | 405 | −2.641 | −21.373 | 4.621 | 1.00 | 0.00 | 3A7 |
| ATOM | 2911 | C | PRO | 405 | −3.739 | −23.646 | 6.756 | 1.00 | 0.00 | 3A7 |
| ATOM | 2912 | O | PRO | 405 | −4.766 | −23.734 | 7.420 | 1.00 | 0.00 | 3A7 |
| ATOM | 2913 | N | LYS | 406 | −3.297 | −24.674 | 5.989 | 1.00 | 0.00 | 3A7 |
| ATOM | 2914 | CA | LYS | 406 | −3.983 | −25.942 | 5.892 | 1.00 | 0.00 | 3A7 |
| ATOM | 2915 | CB | LYS | 406 | −3.545 | −26.748 | 4.646 | 1.00 | 0.00 | 3A7 |
| ATOM | 2916 | CG | LYS | 406 | −3.805 | −26.004 | 3.326 | 1.00 | 0.00 | 3A7 |
| ATOM | 2917 | CD | LYS | 406 | −3.234 | −26.692 | 2.072 | 1.00 | 0.00 | 3A7 |
| ATOM | 2918 | CE | LYS | 406 | −3.939 | −27.993 | 1.650 | 1.00 | 0.00 | 3A7 |
| ATOM | 2919 | NZ | LYS | 406 | −3.601 | −29.127 | 2.541 | 1.00 | 0.00 | 3A7 |
| ATOM | 2920 | C | LYS | 406 | −3.730 | −26.791 | 7.115 | 1.00 | 0.00 | 3A7 |
| ATOM | 2921 | O | LYS | 406 | −4.554 | −27.622 | 7.479 | 1.00 | 0.00 | 3A7 |
| ATOM | 2922 | N | TYR | 407 | −2.572 | −26.584 | 7.783 | 1.00 | 0.00 | 3A7 |
| ATOM | 2923 | CA | TYR | 407 | −2.174 | −27.340 | 8.948 | 1.00 | 0.00 | 3A7 |
| ATOM | 2924 | CB | TYR | 407 | −0.677 | −27.703 | 8.898 | 1.00 | 0.00 | 3A7 |
| ATOM | 2925 | CG | TYR | 407 | −0.579 | −28.906 | 8.000 | 1.00 | 0.00 | 3A7 |
| ATOM | 2926 | CD1 | TYR | 407 | −0.674 | −28.783 | 6.613 | 1.00 | 0.00 | 3A7 |
| ATOM | 2927 | CD2 | TYR | 407 | −0.492 | −30.184 | 8.557 | 1.00 | 0.00 | 3A7 |
| ATOM | 2928 | CE1 | TYR | 407 | −0.693 | −29.912 | 5.799 | 1.00 | 0.00 | 3A7 |
| ATOM | 2929 | CE2 | TYR | 407 | −0.483 | −31.314 | 7.746 | 1.00 | 0.00 | 3A7 |
| ATOM | 2930 | CZ | TYR | 407 | −0.585 | −31.179 | 6.365 | 1.00 | 0.00 | 3A7 |
| ATOM | 2931 | OH | TYR | 407 | −0.583 | −32.324 | 5.538 | 1.00 | 0.00 | 3A7 |
| ATOM | 2932 | C | TYR | 407 | −2.474 | −26.584 | 10.218 | 1.00 | 0.00 | 3A7 |
| ATOM | 2933 | O | TYR | 407 | −2.827 | −27.189 | 11.229 | 1.00 | 0.00 | 3A7 |
| ATOM | 2934 | N | TRP | 408 | −2.334 | −25.240 | 10.202 | 1.00 | 0.00 | 3A7 |
| ATOM | 2935 | CA | TRP | 408 | −2.553 | −24.414 | 11.368 | 1.00 | 0.00 | 3A7 |
| ATOM | 2936 | CB | TRP | 408 | −1.243 | −23.786 | 11.891 | 1.00 | 0.00 | 3A7 |
| ATOM | 2937 | CG | TRP | 408 | −0.242 | −24.837 | 12.291 | 1.00 | 0.00 | 3A7 |
| ATOM | 2938 | CD2 | TRP | 408 | −0.524 | −25.818 | 13.304 | 1.00 | 0.00 | 3A7 |
| ATOM | 2939 | CD1 | TRP | 408 | 1.005 | −25.107 | 11.823 | 1.00 | 0.00 | 3A7 |
| ATOM | 2940 | NE1 | TRP | 408 | 1.515 | −26.195 | 12.482 | 1.00 | 0.00 | 3A7 |
| ATOM | 2941 | CE2 | TRP | 408 | 0.574 | −26.677 | 13.353 | 1.00 | 0.00 | 3A7 |
| ATOM | 2942 | CE3 | TRP | 408 | −1.619 | −26.011 | 14.121 | 1.00 | 0.00 | 3A7 |
| ATOM | 2943 | CZ2 | TRP | 408 | 0.578 | −27.783 | 14.170 | 1.00 | 0.00 | 3A7 |
| ATOM | 2944 | CZ3 | TRP | 408 | −1.597 | −27.102 | 14.983 | 1.00 | 0.00 | 3A7 |
| ATOM | 2945 | CH2 | TRP | 408 | −0.524 | −27.989 | 14.995 | 1.00 | 0.00 | 3A7 |
| ATOM | 2946 | C | TRP | 408 | −3.541 | −23.347 | 10.974 | 1.00 | 0.00 | 3A7 |
| ATOM | 2947 | O | TRP | 408 | −3.197 | −22.324 | 10.383 | 1.00 | 0.00 | 3A7 |
| ATOM | 2948 | N | THR | 409 | −4.838 | −23.556 | 11.320 | 1.00 | 0.00 | 3A7 |
| ATOM | 2949 | CA | THR | 409 | −5.897 | −22.587 | 11.078 | 1.00 | 0.00 | 3A7 |
| ATOM | 2950 | CB | THR | 409 | −7.275 | −23.197 | 11.275 | 1.00 | 0.00 | 3A7 |
| ATOM | 2951 | OG1 | THR | 409 | −8.322 | −22.316 | 10.873 | 1.00 | 0.00 | 3A7 |
| ATOM | 2952 | CG2 | THR | 409 | −7.503 | −23.671 | 12.727 | 1.00 | 0.00 | 3A7 |
| ATOM | 2953 | C | THR | 409 | −5.686 | −21.354 | 11.951 | 1.00 | 0.00 | 3A7 |
| ATOM | 2954 | O | THR | 409 | −5.295 | −21.439 | 13.116 | 1.00 | 0.00 | 3A7 |
| ATOM | 2955 | N | GLU | 410 | −5.888 | −20.153 | 11.363 | 1.00 | 0.00 | 3A7 |
| ATOM | 2956 | CA | GLU | 410 | −5.519 | −18.880 | 11.957 | 1.00 | 0.00 | 3A7 |
| ATOM | 2957 | CB | GLU | 410 | −6.432 | −18.485 | 13.138 | 1.00 | 0.00 | 3A7 |
| ATOM | 2958 | CG | GLU | 410 | −7.920 | −18.447 | 12.751 | 1.00 | 0.00 | 3A7 |
| ATOM | 2959 | CD | GLU | 410 | −8.744 | −18.043 | 13.970 | 1.00 | 0.00 | 3A7 |
| ATOM | 2960 | OE1 | GLU | 410 | −8.705 | −18.790 | 14.985 | 1.00 | 0.00 | 3A7 |
| ATOM | 2961 | OE2 | GLU | 410 | −9.423 | −16.984 | 13.903 | 1.00 | 0.00 | 3A7 |
| ATOM | 2962 | C | GLU | 410 | −4.059 | −18.872 | 12.399 | 1.00 | 0.00 | 3A7 |
| ATOM | 2963 | O | GLU | 410 | −3.756 | −18.654 | 13.572 | 1.00 | 0.00 | 3A7 |
| ATOM | 2964 | N | PRO | 411 | −3.140 | −19.188 | 11.476 | 1.00 | 0.00 | 3A7 |
| ATOM | 2965 | CA | PRO | 411 | −1.722 | −19.438 | 11.747 | 1.00 | 0.00 | 3A7 |
| ATOM | 2966 | CD | PRO | 411 | −3.394 | −19.045 | 10.041 | 1.00 | 0.00 | 3A7 |
| ATOM | 2967 | CB | PRO | 411 | −1.144 | −19.794 | 10.370 | 1.00 | 0.00 | 3A7 |
| ATOM | 2968 | CG | PRO | 411 | −2.020 | −19.039 | 9.368 | 1.00 | 0.00 | 3A7 |
| ATOM | 2969 | C | PRO | 411 | −1.022 | −18.216 | 12.290 | 1.00 | 0.00 | 3A7 |
| ATOM | 2970 | O | PRO | 411 | 0.020 | −18.335 | 12.931 | 1.00 | 0.00 | 3A7 |
| ATOM | 2971 | N | GLU | 412 | −1.592 | −17.019 | 12.058 | 1.00 | 0.00 | 3A7 |
| ATOM | 2972 | CA | GLU | 412 | −1.018 | −15.791 | 12.493 | 1.00 | 0.00 | 3A7 |
| ATOM | 2973 | CB | GLU | 412 | −1.368 | −14.650 | 11.516 | 1.00 | 0.00 | 3A7 |
| ATOM | 2974 | CG | GLU | 412 | −0.914 | −14.895 | 10.063 | 1.00 | 0.00 | 3A7 |
| ATOM | 2975 | CD | GLU | 412 | 0.609 | −14.849 | 9.920 | 1.00 | 0.00 | 3A7 |
| ATOM | 2976 | OE1 | GLU | 412 | 1.093 | −15.190 | 8.807 | 1.00 | 0.00 | 3A7 |
| ATOM | 2977 | OE2 | GLU | 412 | 1.306 | −14.468 | 10.897 | 1.00 | 0.00 | 3A7 |
| ATOM | 2978 | C | GLU | 412 | −1.450 | −15.419 | 13.903 | 1.00 | 0.00 | 3A7 |
| ATOM | 2979 | O | GLU | 412 | −1.079 | −14.360 | 14.405 | 1.00 | 0.00 | 3A7 |
| ATOM | 2980 | N | LYS | 413 | −2.226 | −16.290 | 14.589 | 1.00 | 0.00 | 3A7 |
| ATOM | 2981 | CA | LYS | 413 | −2.691 | −16.035 | 15.932 | 1.00 | 0.00 | 3A7 |
| ATOM | 2982 | CB | LYS | 413 | −4.204 | −16.268 | 16.093 | 1.00 | 0.00 | 3A7 |
| ATOM | 2983 | CG | LYS | 413 | −5.059 | −15.300 | 15.276 | 1.00 | 0.00 | 3A7 |
| ATOM | 2984 | CD | LYS | 413 | −6.569 | −15.457 | 15.519 | 1.00 | 0.00 | 3A7 |
| ATOM | 2985 | CE | LYS | 413 | −7.012 | −15.075 | 16.938 | 1.00 | 0.00 | 3A7 |
| ATOM | 2986 | NZ | LYS | 413 | −8.477 | −15.230 | 17.084 | 1.00 | 0.00 | 3A7 |
| ATOM | 2987 | C | LYS | 413 | −1.939 | −16.896 | 16.917 | 1.00 | 0.00 | 3A7 |
| ATOM | 2988 | O | LYS | 413 | −1.731 | −18.089 | 16.718 | 1.00 | 0.00 | 3A7 |
| ATOM | 2989 | N | PHE | 414 | −1.550 | −16.288 | 18.059 | 1.00 | 0.00 | 3A7 |
| ATOM | 2990 | CA | PHE | 414 | −0.981 | −16.968 | 19.200 | 1.00 | 0.00 | 3A7 |
| ATOM | 2991 | CB | PHE | 414 | −0.298 | −15.950 | 20.125 | 1.00 | 0.00 | 3A7 |
| ATOM | 2992 | CG | PHE | 414 | 0.563 | −16.580 | 21.179 | 1.00 | 0.00 | 3A7 |
| ATOM | 2993 | CD1 | PHE | 414 | 1.763 | −17.214 | 20.878 | 1.00 | 0.00 | 3A7 |
| ATOM | 2994 | CD2 | PHE | 414 | 0.126 | −16.541 | 22.505 | 1.00 | 0.00 | 3A7 |
| ATOM | 2995 | CE1 | PHE | 414 | 2.507 | −17.815 | 21.887 | 1.00 | 0.00 | 3A7 |
| ATOM | 2996 | CE2 | PHE | 414 | 0.864 | −17.148 | 23.513 | 1.00 | 0.00 | 3A7 |
| ATOM | 2997 | CZ | PHE | 414 | 2.048 | −17.802 | 23.199 | 1.00 | 0.00 | 3A7 |
| ATOM | 2998 | C | PHE | 414 | −2.045 | −17.668 | 19.981 | 1.00 | 0.00 | 3A7 |
| ATOM | 2999 | O | PHE | 414 | −2.819 | −17.039 | 20.697 | 1.00 | 0.00 | 3A7 |
| ATOM | 3000 | N | LEU | 415 | −2.093 | −19.006 | 19.847 | 1.00 | 0.00 | 3A7 |
| ATOM | 3001 | CA | LEU | 415 | −3.087 | −19.816 | 20.488 | 1.00 | 0.00 | 3A7 |
| ATOM | 3002 | CB | LEU | 415 | −4.201 | −20.254 | 19.504 | 1.00 | 0.00 | 3A7 |
| ATOM | 3003 | CG | LEU | 415 | −5.081 | −19.105 | 18.954 | 1.00 | 0.00 | 3A7 |
| ATOM | 3004 | CD1 | LEU | 415 | −5.988 | −19.603 | 17.812 | 1.00 | 0.00 | 3A7 |
| ATOM | 3005 | CD2 | LEU | 415 | −5.911 | −18.424 | 20.058 | 1.00 | 0.00 | 3A7 |
| ATOM | 3006 | C | LEU | 415 | −2.421 | −21.064 | 20.998 | 1.00 | 0.00 | 3A7 |
| ATOM | 3007 | O | LEU | 415 | −2.308 | −22.038 | 20.252 | 1.00 | 0.00 | 3A7 |
| ATOM | 3008 | N | PRO | 416 | −2.010 | −21.141 | 22.271 | 1.00 | 0.00 | 3A7 |
| ATOM | 3009 | CA | PRO | 416 | −1.339 | −22.301 | 22.847 | 1.00 | 0.00 | 3A7 |
| ATOM | 3010 | CD | PRO | 416 | −1.874 | −19.967 | 23.122 | 1.00 | 0.00 | 3A7 |
| ATOM | 3011 | CB | PRO | 416 | −1.071 | −21.888 | 24.299 | 1.00 | 0.00 | 3A7 |
| ATOM | 3012 | CG | PRO | 416 | −0.878 | −20.375 | 24.207 | 1.00 | 0.00 | 3A7 |
| ATOM | 3013 | C | PRO | 416 | −2.201 | −23.547 | 22.780 | 1.00 | 0.00 | 3A7 |
| ATOM | 3014 | O | PRO | 416 | −1.669 | −24.651 | 22.766 | 1.00 | 0.00 | 3A7 |
| ATOM | 3015 | N | GLU | 417 | −3.535 | −23.381 | 22.682 | 1.00 | 0.00 | 3A7 |
| ATOM | 3016 | CA | GLU | 417 | −4.480 | −24.458 | 22.678 | 1.00 | 0.00 | 3A7 |
| ATOM | 3017 | CB | GLU | 417 | −5.921 | −23.933 | 22.878 | 1.00 | 0.00 | 3A7 |
| ATOM | 3018 | CG | GLU | 417 | −6.072 | −23.126 | 24.179 | 1.00 | 0.00 | 3A7 |
| ATOM | 3019 | CD | GLU | 417 | −7.521 | −22.672 | 24.323 | 1.00 | 0.00 | 3A7 |
| ATOM | 3020 | OE1 | GLU | 417 | −8.172 | −23.077 | 25.322 | 1.00 | 0.00 | 3A7 |
| ATOM | 3021 | OE2 | GLU | 417 | −7.994 | −21.911 | 23.436 | 1.00 | 0.00 | 3A7 |
| ATOM | 3022 | C | GLU | 417 | −4.459 | −25.295 | 21.427 | 1.00 | 0.00 | 3A7 |
| ATOM | 3023 | O | GLU | 417 | −4.883 | −26.446 | 21.478 | 1.00 | 0.00 | 3A7 |
| ATOM | 3024 | N | ARG | 418 | −3.988 | −24.733 | 20.282 | 1.00 | 0.00 | 3A7 |
| ATOM | 3025 | CA | ARG | 418 | −4.140 | −25.279 | 18.944 | 1.00 | 0.00 | 3A7 |
| ATOM | 3026 | CB | ARG | 418 | −3.753 | −24.206 | 17.890 | 1.00 | 0.00 | 3A7 |
| ATOM | 3027 | CG | ARG | 418 | −4.118 | −24.508 | 16.422 | 1.00 | 0.00 | 3A7 |
| ATOM | 3028 | CD | ARG | 418 | −5.605 | −24.343 | 16.069 | 1.00 | 0.00 | 3A7 |
| ATOM | 3029 | NE | ARG | 418 | −6.397 | −25.466 | 16.668 | 1.00 | 0.00 | 3A7 |
| ATOM | 3030 | CZ | ARG | 418 | −1.765 | −25.471 | 16.670 | 1.00 | 0.00 | 3A7 |
| ATOM | 3031 | NH1 | ARG | 418 | −8.433 | −26.503 | 17.264 | 1.00 | 0.00 | 3A7 |
| ATOM | 3032 | NH2 | ARG | 418 | −8.465 | −24.456 | 16.087 | 1.00 | 0.00 | 3A7 |
| ATOM | 3033 | C | ARG | 418 | −3.324 | −26.538 | 18.706 | 1.00 | 0.00 | 3A7 |
| ATOM | 3034 | O | ARG | 418 | −2.209 | −26.489 | 18.187 | 1.00 | 0.00 | 3A7 |
| ATOM | 3035 | N | PHE | 419 | −3.919 | −27.699 | 19.103 | 1.00 | 0.00 | 3A7 |
| ATOM | 3036 | CA | PHE | 419 | −3.457 | −29.076 | 18.984 | 1.00 | 0.00 | 3A7 |
| ATOM | 3037 | CB | PHE | 419 | −3.390 | −29.565 | 17.516 | 1.00 | 0.00 | 3A7 |
| ATOM | 3038 | CG | PHE | 419 | −4.772 | −29.517 | 16.915 | 1.00 | 0.00 | 3A7 |
| ATOM | 3039 | CD1 | PHE | 419 | −5.049 | −28.723 | 15.799 | 1.00 | 0.00 | 3A7 |
| ATOM | 3040 | CD2 | PHE | 419 | −5.806 | −30.278 | 17.466 | 1.00 | 0.00 | 3A7 |
| ATOM | 3041 | CE1 | PHE | 419 | −6.325 | −28.691 | 15.246 | 1.00 | 0.00 | 3A7 |
| ATOM | 3042 | CE2 | PHE | 419 | −7.085 | −30.245 | 16.919 | 1.00 | 0.00 | 3A7 |
| ATOM | 3043 | CZ | PHE | 419 | −7.345 | −29.452 | 15.806 | 1.00 | 0.00 | 3A7 |
| ATOM | 3044 | C | PHE | 419 | −2.135 | −29.334 | 19.681 | 1.00 | 0.00 | 3A7 |
| ATOM | 3045 | O | PHE | 419 | −1.066 | −28.961 | 19.199 | 1.00 | 0.00 | 3A7 |
| ATOM | 3046 | N | SER | 420 | −2.220 | −30.000 | 20.861 | 1.00 | 0.00 | 3A7 |
| ATOM | 3047 | CA | SER | 420 | −1.120 | −30.259 | 21.764 | 1.00 | 0.00 | 3A7 |
| ATOM | 3048 | CB | SER | 420 | −1.589 | −30.137 | 23.241 | 1.00 | 0.00 | 3A7 |
| ATOM | 3049 | OG | SER | 420 | −0.513 | −30.208 | 24.172 | 1.00 | 0.00 | 3A7 |
| ATOM | 3050 | C | SER | 420 | −0.568 | −31.644 | 21.508 | 1.00 | 0.00 | 3A7 |
| ATOM | 3051 | O | SER | 420 | −1.267 | −32.527 | 21.013 | 1.00 | 0.00 | 3A7 |
| ATOM | 3052 | N | LYS | 421 | 0.722 | −31.842 | 21.877 | 1.00 | 0.00 | 3A7 |
| ATOM | 3053 | CA | LYS | 421 | 1.404 | −33.112 | 21.813 | 1.00 | 0.00 | 3A7 |
| ATOM | 3054 | CB | LYS | 421 | 2.506 | −33.196 | 20.717 | 1.00 | 0.00 | 3A7 |
| ATOM | 3055 | CG | LYS | 421 | 3.804 | −32.380 | 20.921 | 1.00 | 0.00 | 3A7 |
| ATOM | 3056 | CD | LYS | 421 | 3.679 | −30.845 | 20.996 | 1.00 | 0.00 | 3A7 |
| ATOM | 3057 | CE | LYS | 421 | 3.332 | −30.147 | 19.673 | 1.00 | 0.00 | 3A7 |
| ATOM | 3058 | NZ | LYS | 421 | 1.927 | −30.385 | 19.273 | 1.00 | 0.00 | 3A7 |
| ATOM | 3059 | C | LYS | 421 | 2.007 | −33.350 | 23.167 | 1.00 | 0.00 | 3A7 |
| ATOM | 3060 | O | LYS | 421 | 2.165 | −32.424 | 23.962 | 1.00 | 0.00 | 3A7 |
| ATOM | 3061 | N | LYS | 422 | 2.373 | −34.620 | 23.448 | 1.00 | 0.00 | 3A7 |
| ATOM | 3062 | CA | LYS | 422 | 3.032 | −34.986 | 24.675 | 1.00 | 0.00 | 3A7 |
| ATOM | 3063 | CB | LYS | 422 | 2.048 | −35.203 | 25.856 | 1.00 | 0.00 | 3A7 |
| ATOM | 3064 | CG | LYS | 422 | 2.715 | −35.433 | 27.225 | 1.00 | 0.00 | 3A7 |
| ATOM | 3065 | CD | LYS | 422 | 3.520 | −34.222 | 27.727 | 1.00 | 0.00 | 3A7 |
| ATOM | 3066 | CE | LYS | 422 | 4.151 | −34.436 | 29.109 | 1.00 | 0.00 | 3A7 |
| ATOM | 3067 | NZ | LYS | 422 | 5.140 | −35.537 | 29.071 | 1.00 | 0.00 | 3A7 |
| ATOM | 3068 | C | LYS | 422 | 3.767 | −36.262 | 24.379 | 1.00 | 0.00 | 3A7 |
| ATOM | 3069 | O | LYS | 422 | 4.874 | −36.483 | 24.867 | 1.00 | 0.00 | 3A7 |
| ATOM | 3070 | N | ASN | 423 | 3.138 | −37.137 | 23.555 | 1.00 | 0.00 | 3A7 |
| ATOM | 3071 | CA | ASN | 423 | 3.658 | −38.427 | 23.165 | 1.00 | 0.00 | 3A7 |
| ATOM | 3072 | CB | ASN | 423 | 2.646 | −39.581 | 23.442 | 1.00 | 0.00 | 3A7 |
| ATOM | 3073 | CG | ASN | 423 | 1.264 | −39.329 | 22.812 | 1.00 | 0.00 | 3A7 |
| ATOM | 3074 | OD1 | ASN | 423 | 0.498 | −38.476 | 23.277 | 1.00 | 0.00 | 3A7 |
| ATOM | 3075 | ND2 | ASN | 423 | 0.950 | −40.110 | 21.734 | 1.00 | 0.00 | 3A7 |
| ATOM | 3076 | C | ASN | 423 | 4.031 | −38.367 | 21.703 | 1.00 | 0.00 | 3A7 |
| ATOM | 3077 | O | ASN | 423 | 3.916 | −37.323 | 21.061 | 1.00 | 0.00 | 3A7 |
| ATOM | 3078 | N | LYS | 424 | 4.481 | −39.522 | 21.148 | 1.00 | 0.00 | 3A7 |
| ATOM | 3079 | CA | LYS | 424 | 4.861 | −39.684 | 19.760 | 1.00 | 0.00 | 3A7 |
| ATOM | 3080 | CB | LYS | 424 | 5.998 | −40.724 | 19.591 | 1.00 | 0.00 | 3A7 |
| ATOM | 3081 | CG | LYS | 424 | 6.530 | −40.859 | 18.153 | 1.00 | 0.00 | 3A7 |
| ATOM | 3082 | CD | LYS | 424 | 7.676 | −41.874 | 18.013 | 1.00 | 0.00 | 3A7 |
| ATOM | 3083 | CE | LYS | 424 | 8.960 | −41.460 | 18.744 | 1.00 | 0.00 | 3A7 |
| ATOM | 3084 | NZ | LYS | 424 | 10.027 | −42.465 | 18.534 | 1.00 | 0.00 | 3A7 |
| ATOM | 3085 | C | LYS | 424 | 3.649 | −40.125 | 18.981 | 1.00 | 0.00 | 3A7 |
| ATOM | 3086 | O | LYS | 424 | 3.011 | −41.116 | 19.335 | 1.00 | 0.00 | 3A7 |
| ATOM | 3087 | N | ASP | 425 | 3.321 | −39.353 | 17.910 | 1.00 | 0.00 | 3A7 |
| ATOM | 3088 | CA | ASP | 425 | 2.171 | −39.492 | 17.032 | 1.00 | 0.00 | 3A7 |
| ATOM | 3089 | CB | ASP | 425 | 1.794 | −40.959 | 16.658 | 1.00 | 0.00 | 3A7 |
| ATOM | 3090 | CG | ASP | 425 | 0.795 | −41.005 | 15.496 | 1.00 | 0.00 | 3A7 |
| ATOM | 3091 | OD1 | ASP | 425 | −0.322 | −41.550 | 15.698 | 1.00 | 0.00 | 3A7 |
| ATOM | 3092 | OD2 | ASP | 425 | 1.142 | −40.497 | 14.397 | 1.00 | 0.00 | 3A7 |
| ATOM | 3093 | C | ASP | 425 | 1.009 | −38.771 | 17.679 | 1.00 | 0.00 | 3A7 |
| ATOM | 3094 | O | ASP | 425 | 0.241 | −39.355 | 18.444 | 1.00 | 0.00 | 3A7 |
| ATOM | 3095 | N | ASN | 426 | 0.881 | −37.457 | 17.372 | 1.00 | 0.00 | 3A7 |
| ATOM | 3096 | CA | ASN | 426 | −0.110 | −36.573 | 17.945 | 1.00 | 0.00 | 3A7 |
| ATOM | 3097 | CB | ASN | 426 | 0.515 | −35.250 | 18.487 | 1.00 | 0.00 | 3A7 |
| ATOM | 3098 | CG | ASN | 426 | 1.320 | −34.458 | 17.438 | 1.00 | 0.00 | 3A7 |
| ATOM | 3099 | OD1 | ASN | 426 | 2.282 | −34.963 | 16.848 | 1.00 | 0.00 | 3A7 |
| ATOM | 3100 | ND2 | ASN | 426 | 0.912 | −33.168 | 17.234 | 1.00 | 0.00 | 3A7 |
| ATOM | 3101 | C | ASN | 426 | −1.189 | −36.329 | 16.915 | 1.00 | 0.00 | 3A7 |
| ATOM | 3102 | O | ASN | 426 | −1.547 | −37.229 | 16.157 | 1.00 | 0.00 | 3A7 |
| ATOM | 3103 | N | ILE | 427 | −1.746 | −35.090 | 16.880 | 1.00 | 0.00 | 3A7 |
| ATOM | 3104 | CA | ILE | 427 | −2.849 | −34.709 | 16.022 | 1.00 | 0.00 | 3A7 |
| ATOM | 3105 | CB | ILE | 427 | −3.731 | −33.632 | 16.647 | 1.00 | 0.00 | 3A7 |
| ATOM | 3106 | CG2 | ILE | 427 | −4.975 | −33.389 | 15.762 | 1.00 | 0.00 | 3A7 |
| ATOM | 3107 | CG1 | ILE | 427 | −4.130 | −34.009 | 18.097 | 1.00 | 0.00 | 3A7 |
| ATOM | 3108 | CD | ILE | 427 | −4.944 | −35.302 | 18.218 | 1.00 | 0.00 | 3A7 |
| ATOM | 3109 | C | ILE | 427 | −2.306 | −34.224 | 14.699 | 1.00 | 0.00 | 3A7 |
| ATOM | 3110 | O | ILE | 427 | −2.782 | −34.635 | 13.641 | 1.00 | 0.00 | 3A7 |
| ATOM | 3111 | N | ASP | 428 | −1.289 | −33.326 | 14.741 | 1.00 | 0.00 | 3A7 |
| ATOM | 3112 | CA | ASP | 428 | −0.691 | −32.752 | 13.555 | 1.00 | 0.00 | 3A7 |
| ATOM | 3113 | CB | ASP | 428 | −1.067 | −31.262 | 13.360 | 1.00 | 0.00 | 3A7 |
| ATOM | 3114 | CG | ASP | 428 | −2.578 | −31.131 | 13.173 | 1.00 | 0.00 | 3A7 |
| ATOM | 3115 | OD1 | ASP | 428 | −3.101 | −31.683 | 12.169 | 1.00 | 0.00 | 3A7 |
| ATOM | 3116 | OD2 | ASP | 428 | −3.224 | −30.473 | 14.030 | 1.00 | 0.00 | 3A7 |
| ATOM | 3117 | C | ASP | 428 | 0.815 | −32.877 | 13.650 | 1.00 | 0.00 | 3A7 |
| ATOM | 3118 | O | ASP | 428 | 1.471 | −31.935 | 14.093 | 1.00 | 0.00 | 3A7 |
| ATOM | 3119 | N | PRO | 429 | 1.414 | −34.003 | 13.251 | 1.00 | 0.00 | 3A7 |
| ATOM | 3120 | CA | PRO | 429 | 2.856 | −34.177 | 13.157 | 1.00 | 0.00 | 3A7 |
| ATOM | 3121 | CD | PRO | 429 | 0.694 | −35.245 | 12.998 | 1.00 | 0.00 | 3A7 |
| ATOM | 3122 | CB | PRO | 429 | 3.026 | −35.701 | 13.258 | 1.00 | 0.00 | 3A7 |
| ATOM | 3123 | CG | PRO | 429 | 1.763 | −36.266 | 12.604 | 1.00 | 0.00 | 3A7 |
| ATOM | 3124 | C | PRO | 429 | 3.490 | −33.658 | 11.858 | 1.00 | 0.00 | 3A7 |
| ATOM | 3125 | O | PRO | 429 | 2.892 | −33.616 | 10.780 | 1.00 | 0.00 | 3A7 |
| ATOM | 3126 | N | TYR | 430 | 4.793 | −33.325 | 11.967 | 1.00 | 0.00 | 3A7 |
| ATOM | 3127 | CA | TYR | 430 | 5.745 | −33.153 | 10.884 | 1.00 | 0.00 | 3A7 |
| ATOM | 3128 | CB | TYR | 430 | 5.621 | −34.248 | 9.770 | 1.00 | 0.00 | 3A7 |
| ATOM | 3129 | CG | TYR | 430 | 6.889 | −34.285 | 8.969 | 1.00 | 0.00 | 3A7 |
| ATOM | 3130 | CD1 | TYR | 430 | 8.048 | −34.836 | 9.516 | 1.00 | 0.00 | 3A7 |
| ATOM | 3131 | CD2 | TYR | 430 | 6.967 | −33.566 | 7.779 | 1.00 | 0.00 | 3A7 |
| ATOM | 3132 | CE1 | TYR | 430 | 9.284 | −34.596 | 8.926 | 1.00 | 0.00 | 3A7 |
| ATOM | 3133 | CE2 | TYR | 430 | 8.205 | −33.267 | 7.232 | 1.00 | 0.00 | 3A7 |
| ATOM | 3134 | CZ | TYR | 430 | 9.366 | −33.774 | 7.806 | 1.00 | 0.00 | 3A7 |
| ATOM | 3135 | OH | TYR | 430 | 10.620 | −33.421 | 7.266 | 1.00 | 0.00 | 3A7 |
| ATOM | 3136 | C | TYR | 430 | 5.763 | −31.761 | 10.274 | 1.00 | 0.00 | 3A7 |
| ATOM | 3137 | O | TYR | 430 | 6.683 | −31.428 | 9.529 | 1.00 | 0.00 | 3A7 |
| ATOM | 3138 | N | ILE | 431 | 4.795 | −30.877 | 10.599 | 1.00 | 0.00 | 3A7 |
| ATOM | 3139 | CA | ILE | 431 | 4.842 | −29.502 | 10.146 | 1.00 | 0.00 | 3A7 |
| ATOM | 3140 | CB | ILE | 431 | 3.495 | −28.893 | 9.784 | 1.00 | 0.00 | 3A7 |
| ATOM | 3141 | CG2 | ILE | 431 | 2.954 | −29.661 | 8.570 | 1.00 | 0.00 | 3A7 |
| ATOM | 3142 | CG1 | ILE | 431 | 2.477 | −28.800 | 10.949 | 1.00 | 0.00 | 3A7 |
| ATOM | 3143 | CD | ILE | 431 | 1.966 | −30.124 | 11.513 | 1.00 | 0.00 | 3A7 |
| ATOM | 3144 | C | ILE | 431 | 5.480 | −28.630 | 11.193 | 1.00 | 0.00 | 3A7 |
| ATOM | 3145 | O | ILE | 431 | 5.889 | −27.509 | 10.893 | 1.00 | 0.00 | 3A7 |
| ATOM | 3146 | N | TYR | 432 | 5.562 | −29.122 | 12.460 | 1.00 | 0.00 | 3A7 |
| ATOM | 3147 | CA | TYR | 432 | 6.198 | −28.443 | 13.571 | 1.00 | 0.00 | 3A7 |
| ATOM | 3148 | CB | TYR | 432 | 5.758 | −28.969 | 14.973 | 1.00 | 0.00 | 3A7 |
| ATOM | 3149 | CG | TYR | 432 | 4.498 | −28.367 | 15.505 | 1.00 | 0.00 | 3A7 |
| ATOM | 3150 | CD1 | TYR | 432 | 3.438 | −29.190 | 15.886 | 1.00 | 0.00 | 3A7 |
| ATOM | 3151 | CD2 | TYR | 432 | 4.439 | −27.004 | 15.799 | 1.00 | 0.00 | 3A7 |
| ATOM | 3152 | CE1 | TYR | 432 | 2.350 | −28.665 | 16.575 | 1.00 | 0.00 | 3A7 |
| ATOM | 3153 | CE2 | TYR | 432 | 3.348 | −26.477 | 16.484 | 1.00 | 0.00 | 3A7 |
| ATOM | 3154 | CZ | TYR | 432 | 2.304 | −27.307 | 16.878 | 1.00 | 0.00 | 3A7 |
| ATOM | 3155 | OH | TYR | 432 | 1.204 | −26.773 | 17.586 | 1.00 | 0.00 | 3A7 |
| ATOM | 3156 | C | TYR | 432 | 7.676 | −28.741 | 13.531 | 1.00 | 0.00 | 3A7 |
| ATOM | 3157 | O | TYR | 432 | 8.161 | −29.675 | 14.169 | 1.00 | 0.00 | 3A7 |
| ATOM | 3158 | N | THR | 433 | 8.425 | −27.919 | 12.778 | 1.00 | 0.00 | 3A7 |
| ATOM | 3159 | CA | THR | 433 | 9.824 | −28.119 | 12.506 | 1.00 | 0.00 | 3A7 |
| ATOM | 3160 | CB | THR | 433 | 10.074 | −28.167 | 10.995 | 1.00 | 0.00 | 3A7 |
| ATOM | 3161 | OG1 | THR | 433 | 9.638 | −26.980 | 10.336 | 1.00 | 0.00 | 3A7 |
| ATOM | 3162 | CG2 | THR | 433 | 9.309 | −29.368 | 10.401 | 1.00 | 0.00 | 3A7 |
| ATOM | 3163 | C | THR | 433 | 10.729 | −27.070 | 13.107 | 1.00 | 0.00 | 3A7 |
| ATOM | 3164 | O | THR | 433 | 11.720 | −26.744 | 12.459 | 1.00 | 0.00 | 3A7 |
| ATOM | 3165 | N | PRO | 434 | 10.544 | −26.494 | 14.305 | 1.00 | 0.00 | 3A7 |
| ATOM | 3166 | CA | PRO | 434 | 11.476 | −25.526 | 14.854 | 1.00 | 0.00 | 3A7 |
| ATOM | 3167 | CD | PRO | 434 | 9.603 | −26.944 | 15.329 | 1.00 | 0.00 | 3A7 |
| ATOM | 3168 | CB | PRO | 434 | 10.738 | −25.003 | 16.098 | 1.00 | 0.00 | 3A7 |
| ATOM | 3169 | CG | PRO | 434 | 9.981 | −26.220 | 16.622 | 1.00 | 0.00 | 3A7 |
| ATOM | 3170 | C | PRO | 434 | 12.807 | −26.180 | 15.226 | 1.00 | 0.00 | 3A7 |
| ATOM | 3171 | O | PRO | 434 | 13.809 | −25.477 | 15.300 | 1.00 | 0.00 | 3A7 |
| ATOM | 3172 | N | PHE | 435 | 12.847 | −27.518 | 15.456 | 1.00 | 0.00 | 3A7 |
| ATOM | 3173 | CA | PHE | 435 | 14.041 | −28.253 | 15.844 | 1.00 | 0.00 | 3A7 |
| ATOM | 3174 | CB | PHE | 435 | 13.670 | −29.173 | 17.045 | 1.00 | 0.00 | 3A7 |
| ATOM | 3175 | CG | PHE | 435 | 13.744 | −28.509 | 18.358 | 1.00 | 0.00 | 3A7 |
| ATOM | 3176 | CD1 | PHE | 435 | 14.729 | −27.557 | 18.520 | 1.00 | 0.00 | 3A7 |
| ATOM | 3177 | CD2 | PHE | 435 | 12.731 | −28.261 | 19.269 | 1.00 | 0.00 | 3A7 |
| ATOM | 3178 | CE1 | PHE | 435 | 14.264 | −26.403 | 19.460 | 1.00 | 0.00 | 3A7 |
| ATOM | 3179 | CE2 | PHE | 435 | 14.105 | −27.600 | 20.460 | 1.00 | 0.00 | 3A7 |
| ATOM | 3180 | CZ | PHE | 435 | 13.408 | −26.726 | 20.695 | 1.00 | 0.00 | 3A7 |
| ATOM | 3181 | C | PHE | 435 | 14.509 | −29.099 | 14.689 | 1.00 | 0.00 | 3A7 |
| ATOM | 3182 | O | PHE | 435 | 15.352 | −29.980 | 14.855 | 1.00 | 0.00 | 3A7 |
| ATOM | 3183 | N | GLY | 436 | 13.993 | −28.834 | 13.469 | 1.00 | 0.00 | 3A7 |
| ATOM | 3184 | CA | GLY | 436 | 14.463 | −29.495 | 12.269 | 1.00 | 0.00 | 3A7 |
| ATOM | 3185 | C | GLY | 436 | 13.733 | −30.777 | 12.129 | 1.00 | 0.00 | 3A7 |
| ATOM | 3186 | O | GLY | 436 | 12.792 | −31.061 | 12.870 | 1.00 | 0.00 | 3A7 |
| ATOM | 3187 | N | SER | 437 | 14.147 | −31.599 | 11.152 | 1.00 | 0.00 | 3A7 |
| ATOM | 3188 | CA | SER | 437 | 13.525 | −32.872 | 10.944 | 1.00 | 0.00 | 3A7 |
| ATOM | 3189 | CB | SER | 437 | 12.322 | −32.829 | 9.974 | 1.00 | 0.00 | 3A7 |
| ATOM | 3190 | OG | SER | 437 | 11.247 | −32.079 | 10.518 | 1.00 | 0.00 | 3A7 |
| ATOM | 3191 | C | SER | 437 | 14.585 | −33.730 | 10.351 | 1.00 | 0.00 | 3A7 |
| ATOM | 3192 | O | SER | 437 | 15.352 | −33.291 | 9.494 | 1.00 | 0.00 | 3A7 |
| ATOM | 3193 | N | GLY | 438 | 14.623 | −35.006 | 10.788 | 1.00 | 0.00 | 3A7 |
| ATOM | 3194 | CA | GLY | 438 | 15.462 | −36.010 | 10.184 | 1.00 | 0.00 | 3A7 |
| ATOM | 3195 | C | GLY | 438 | 16.610 | −36.414 | 11.067 | 1.00 | 0.00 | 3A7 |
| ATOM | 3196 | O | GLY | 438 | 16.526 | −36.346 | 12.293 | 1.00 | 0.00 | 3A7 |
| ATOM | 3197 | N | PRO | 439 | 17.704 | −36.873 | 10.443 | 1.00 | 0.00 | 3A7 |
| ATOM | 3198 | CA | PRO | 439 | 18.897 | −37.275 | 11.180 | 1.00 | 0.00 | 3A7 |
| ATOM | 3199 | CD | PRO | 439 | 17.653 | −37.555 | 9.152 | 1.00 | 0.00 | 3A7 |
| ATOM | 3200 | CB | PRO | 439 | 19.741 | −38.079 | 10.170 | 1.00 | 0.00 | 3A7 |
| ATOM | 3201 | CG | PRO | 439 | 18.702 | −38.666 | 9.220 | 1.00 | 0.00 | 3A7 |
| ATOM | 3202 | C | PRO | 439 | 19.700 | −36.119 | 11.729 | 1.00 | 0.00 | 3A7 |
| ATOM | 3203 | O | PRO | 439 | 20.523 | −36.342 | 12.614 | 1.00 | 0.00 | 3A7 |
| ATOM | 3204 | N | ARG | 440 | 19.490 | −34.889 | 11.217 | 1.00 | 0.00 | 3A7 |
| ATOM | 3205 | CA | ARG | 440 | 20.212 | −33.720 | 11.642 | 1.00 | 0.00 | 3A7 |
| ATOM | 3206 | CB | ARG | 440 | 20.704 | −32.913 | 10.433 | 1.00 | 0.00 | 3A7 |
| ATOM | 3207 | CG | ARG | 440 | 21.775 | −33.662 | 9.617 | 1.00 | 0.00 | 3A7 |
| ATOM | 3208 | CD | ARG | 440 | 21.846 | −33.224 | 8.148 | 1.00 | 0.00 | 3A7 |
| ATOM | 3209 | NE | ARG | 440 | 20.618 | −33.741 | 7.458 | 1.00 | 0.00 | 3A7 |
| ATOM | 3210 | CZ | ARG | 440 | 20.391 | −33.552 | 6.123 | 1.00 | 0.00 | 3A7 |
| ATOM | 3211 | NH1 | ARG | 440 | 19.299 | −34.126 | 5.537 | 1.00 | 0.00 | 3A7 |
| ATOM | 3212 | NH2 | ARG | 440 | 21.244 | −32.799 | 5.371 | 1.00 | 0.00 | 3A7 |
| ATOM | 3213 | C | ARG | 440 | 19.307 | −32.855 | 12.480 | 1.00 | 0.00 | 3A7 |
| ATOM | 3214 | O | ARG | 440 | 19.471 | −31.639 | 12.565 | 1.00 | 0.00 | 3A7 |
| ATOM | 3215 | N | ASN | 441 | 18.322 | −33.480 | 13.163 | 1.00 | 0.00 | 3A7 |
| ATOM | 3216 | CA | ASN | 441 | 17.420 | −32.809 | 14.066 | 1.00 | 0.00 | 3A7 |
| ATOM | 3217 | CB | ASN | 441 | 16.263 | −33.734 | 14.515 | 1.00 | 0.00 | 3A7 |
| ATOM | 3218 | CG | ASN | 441 | 16.713 | −34.970 | 15.324 | 1.00 | 0.00 | 3A7 |
| ATOM | 3219 | OD1 | ASN | 441 | 17.527 | −35.781 | 14.869 | 1.00 | 0.00 | 3A7 |
| ATOM | 3220 | ND2 | ASN | 441 | 16.154 | −35.093 | 16.567 | 1.00 | 0.00 | 3A7 |
| ATOM | 3221 | C | ASN | 441 | 18.197 | −32.339 | 15.260 | 1.00 | 0.00 | 3A7 |
| ATOM | 3222 | O | ASN | 441 | 19.263 | −32.884 | 15.550 | 1.00 | 0.00 | 3A7 |
| ATOM | 3223 | N | CYS | 442 | 17.690 | −31.324 | 15.990 | 1.00 | 0.00 | 3A7 |
| ATOM | 3224 | CA | CYS | 442 | 18.409 | −30.739 | 17.097 | 1.00 | 0.00 | 3A7 |
| ATOM | 3225 | CB | CYS | 442 | 17.629 | −29.548 | 17.676 | 1.00 | 0.00 | 3A7 |
| ATOM | 3226 | SG | CYS | 442 | 18.592 | −28.606 | 18.905 | 1.00 | 0.00 | 3A7 |
| ATOM | 3227 | C | CYS | 442 | 18.683 | −31.758 | 18.181 | 1.00 | 0.00 | 3A7 |
| ATOM | 3228 | O | CYS | 442 | 17.779 | −32.410 | 18.699 | 1.00 | 0.00 | 3A7 |
| ATOM | 3229 | N | ILE | 443 | 19.971 | −31.894 | 18.535 | 1.00 | 0.00 | 3A7 |
| ATOM | 3230 | CA | ILE | 443 | 20.405 | −32.830 | 19.531 | 1.00 | 0.00 | 3A7 |
| ATOM | 3231 | CB | ILE | 443 | 21.872 | −33.211 | 19.324 | 1.00 | 0.00 | 3A7 |
| ATOM | 3232 | CG2 | ILE | 443 | 22.414 | −34.095 | 20.472 | 1.00 | 0.00 | 3A7 |
| ATOM | 3233 | CG1 | ILE | 443 | 22.046 | −33.916 | 17.955 | 1.00 | 0.00 | 3A7 |
| ATOM | 3234 | CD | ILE | 443 | 21.378 | −35.291 | 17.849 | 1.00 | 0.00 | 3A7 |
| ATOM | 3235 | C | ILE | 443 | 20.167 | −32.223 | 20.902 | 1.00 | 0.00 | 3A7 |
| ATOM | 3236 | O | ILE | 443 | 20.008 | −32.929 | 21.894 | 1.00 | 0.00 | 3A7 |
| ATOM | 3237 | N | GLY | 444 | 20.106 | −30.876 | 20.975 | 1.00 | 0.00 | 3A7 |
| ATOM | 3238 | CA | GLY | 444 | 19.894 | −30.159 | 22.204 | 1.00 | 0.00 | 3A7 |
| ATOM | 3239 | C | GLY | 444 | 18.471 | −29.893 | 22.535 | 1.00 | 0.00 | 3A7 |
| ATOM | 3240 | O | GLY | 444 | 18.222 | −29.153 | 23.479 | 1.00 | 0.00 | 3A7 |
| ATOM | 3241 | N | MET | 445 | 17.493 | −30.471 | 21.788 | 1.00 | 0.00 | 3A7 |
| ATOM | 3242 | CA | MET | 445 | 16.056 | −30.254 | 21.939 | 1.00 | 0.00 | 3A7 |
| ATOM | 3243 | CB | MET | 445 | 15.279 | −31.201 | 21.002 | 1.00 | 0.00 | 3A7 |
| ATOM | 3244 | CG | MET | 445 | 13.742 | −31.222 | 21.137 | 1.00 | 0.00 | 3A7 |
| ATOM | 3245 | SD | MET | 445 | 12.898 | −32.098 | 19.780 | 1.00 | 0.00 | 3A7 |
| ATOM | 3246 | CE | MET | 445 | 13.584 | −33.749 | 20.107 | 1.00 | 0.00 | 3A7 |
| ATOM | 3247 | C | MET | 445 | 15.558 | −30.454 | 23.349 | 1.00 | 0.00 | 3A7 |
| ATOM | 3248 | O | MET | 445 | 14.887 | −29.598 | 23.919 | 1.00 | 0.00 | 3A7 |
| ATOM | 3249 | N | ARG | 446 | 15.928 | −31.580 | 23.976 | 1.00 | 0.00 | 3A7 |
| ATOM | 3250 | CA | ARG | 446 | 15.476 | −31.910 | 25.309 | 1.00 | 0.00 | 3A7 |
| ATOM | 3251 | CB | ARG | 446 | 15.826 | −33.368 | 25.684 | 1.00 | 0.00 | 3A7 |
| ATOM | 3252 | CG | ARG | 446 | 15.490 | −34.340 | 24.538 | 1.00 | 0.00 | 3A7 |
| ATOM | 3253 | CD | ARG | 446 | 15.588 | −35.824 | 24.911 | 1.00 | 0.00 | 3A7 |
| ATOM | 3254 | NE | ARG | 446 | 14.428 | −36.164 | 25.799 | 1.00 | 0.00 | 3A7 |
| ATOM | 3255 | CZ | ARG | 446 | 14.005 | −37.451 | 25.986 | 1.00 | 0.00 | 3A7 |
| ATOM | 3256 | NH1 | ARG | 446 | 12.921 | −37.696 | 26.780 | 1.00 | 0.00 | 3A7 |
| ATOM | 3257 | NH2 | ARG | 446 | 14.658 | −38.492 | 25.394 | 1.00 | 0.00 | 3A7 |
| ATOM | 3258 | C | ARG | 446 | 16.001 | −30.952 | 26.348 | 1.00 | 0.00 | 3A7 |
| ATOM | 3259 | O | ARG | 446 | 15.276 | −30.542 | 27.248 | 1.00 | 0.00 | 3A7 |
| ATOM | 3260 | N | PHE | 447 | 17.267 | −30.512 | 26.201 | 1.00 | 0.00 | 3A7 |
| ATOM | 3261 | CA | PHE | 447 | 17.865 | −29.566 | 27.113 | 1.00 | 0.00 | 3A7 |
| ATOM | 3262 | CB | PHE | 447 | 19.408 | −29.515 | 27.061 | 1.00 | 0.00 | 3A7 |
| ATOM | 3263 | CG | PHE | 447 | 19.952 | −30.827 | 26.613 | 1.00 | 0.00 | 3A7 |
| ATOM | 3264 | CD1 | PHE | 447 | 20.810 | −30.828 | 25.520 | 1.00 | 0.00 | 3A7 |
| ATOM | 3265 | CD2 | PHE | 447 | 19.433 | −32.037 | 27.086 | 1.00 | 0.00 | 3A7 |
| ATOM | 3266 | CE1 | PHE | 447 | 20.971 | −31.985 | 24.778 | 1.00 | 0.00 | 3A7 |
| ATOM | 3267 | CE2 | PHE | 447 | 19.526 | −33.173 | 26.291 | 1.00 | 0.00 | 3A7 |
| ATOM | 3268 | CZ | PHE | 447 | 20.229 | −33.109 | 25.096 | 1.00 | 0.00 | 3A7 |
| ATOM | 3269 | C | PHE | 447 | 17.285 | −28.185 | 26.918 | 1.00 | 0.00 | 3A7 |
| ATOM | 3270 | O | PHE | 447 | 17.071 | −27.458 | 27.881 | 1.00 | 0.00 | 3A7 |
| ATOM | 3271 | N | ALA | 448 | 16.949 | −27.798 | 25.656 | 1.00 | 0.00 | 3A7 |
| ATOM | 3272 | CA | ALA | 448 | 16.406 | −26.493 | 25.374 | 1.00 | 0.00 | 3A7 |
| ATOM | 3273 | CB | ALA | 448 | 16.379 | −26.216 | 23.882 | 1.00 | 0.00 | 3A7 |
| ATOM | 3274 | C | ALA | 448 | 15.020 | −26.344 | 25.914 | 1.00 | 0.00 | 3A7 |
| ATOM | 3275 | O | ALA | 448 | 14.679 | −25.313 | 26.493 | 1.00 | 0.00 | 3A7 |
| ATOM | 3276 | N | LEU | 449 | 14.197 | −27.406 | 25.800 | 1.00 | 0.00 | 3A7 |
| ATOM | 3277 | CA | LEU | 449 | 12.842 | −27.402 | 26.309 | 1.00 | 0.00 | 3A7 |
| ATOM | 3278 | CB | LEU | 449 | 12.029 | −28.605 | 25.813 | 1.00 | 0.00 | 3A7 |
| ATOM | 3279 | CG | LEU | 449 | 11.639 | −28.523 | 24.324 | 1.00 | 0.00 | 3A7 |
| ATOM | 3280 | CD1 | LEU | 449 | 11.120 | −29.879 | 23.807 | 1.00 | 0.00 | 3A7 |
| ATOM | 3281 | CD2 | LEU | 449 | 10.621 | −27.401 | 24.038 | 1.00 | 0.00 | 3A7 |
| ATOM | 3282 | C | LEU | 449 | 12.800 | −27.422 | 27.811 | 1.00 | 0.00 | 3A7 |
| ATOM | 3283 | O | LEU | 449 | 11.936 | −26.797 | 28.417 | 1.00 | 0.00 | 3A7 |
| ATOM | 3284 | N | VAL | 450 | 13.770 | −28.111 | 28.454 | 1.00 | 0.00 | 3A7 |
| ATOM | 3285 | CA | VAL | 450 | 13.883 | −28.171 | 29.893 | 1.00 | 0.00 | 3A7 |
| ATOM | 3286 | CB | VAL | 450 | 14.890 | −29.213 | 30.345 | 1.00 | 0.00 | 3A7 |
| ATOM | 3287 | CG1 | VAL | 450 | 15.177 | −29.141 | 31.862 | 1.00 | 0.00 | 3A7 |
| ATOM | 3288 | CG2 | VAL | 450 | 14.262 | −30.592 | 30.049 | 1.00 | 0.00 | 3A7 |
| ATOM | 3289 | C | VAL | 450 | 14.256 | −26.828 | 30.431 | 1.00 | 0.00 | 3A7 |
| ATOM | 3290 | O | VAL | 450 | 13.633 | −26.345 | 31.364 | 1.00 | 0.00 | 3A7 |
| ATOM | 3291 | N | ASN | 451 | 15.263 | −26.162 | 29.833 | 1.00 | 0.00 | 3A7 |
| ATOM | 3292 | CA | ASN | 451 | 15.735 | −24.855 | 30.232 | 1.00 | 0.00 | 3A7 |
| ATOM | 3293 | CB | ASN | 451 | 16.873 | −24.401 | 29.295 | 1.00 | 0.00 | 3A7 |
| ATOM | 3294 | CG | ASN | 451 | 18.141 | −25.243 | 29.476 | 1.00 | 0.00 | 3A7 |
| ATOM | 3295 | OD1 | ASN | 451 | 18.233 | −26.089 | 30.369 | 1.00 | 0.00 | 3A7 |
| ATOM | 3296 | ND2 | ASN | 451 | 19.145 | −24.981 | 28.583 | 1.00 | 0.00 | 3A7 |
| ATOM | 3297 | C | ASN | 451 | 14.659 | −23.792 | 30.150 | 1.00 | 0.00 | 3A7 |
| ATOM | 3298 | O | ASN | 451 | 14.440 | −23.023 | 31.081 | 1.00 | 0.00 | 3A7 |
| ATOM | 3299 | N | MET | 452 | 13.918 | −23.759 | 29.019 | 1.00 | 0.00 | 3A7 |
| ATOM | 3300 | CA | MET | 452 | 12.853 | −22.808 | 28.800 | 1.00 | 0.00 | 3A7 |
| ATOM | 3301 | CB | MET | 452 | 12.311 | −22.880 | 27.353 | 1.00 | 0.00 | 3A7 |
| ATOM | 3302 | CG | MET | 452 | 13.202 | −22.090 | 26.370 | 1.00 | 0.00 | 3A7 |
| ATOM | 3303 | SD | MET | 452 | 13.800 | −22.975 | 24.894 | 1.00 | 0.00 | 3A7 |
| ATOM | 3304 | CE | MET | 452 | 12.280 | −23.810 | 24.359 | 1.00 | 0.00 | 3A7 |
| ATOM | 3305 | C | MET | 452 | 11.733 | −22.982 | 29.778 | 1.00 | 0.00 | 3A7 |
| ATOM | 3306 | O | MET | 452 | 11.255 | −22.013 | 30.355 | 1.00 | 0.00 | 3A7 |
| ATOM | 3307 | N | LYS | 453 | 11.310 | −24.236 | 30.042 | 1.00 | 0.00 | 3A7 |
| ATOM | 3308 | CA | LYS | 453 | 10.240 | −24.536 | 30.965 | 1.00 | 0.00 | 3A7 |
| ATOM | 3309 | CB | LYS | 453 | 9.864 | −26.021 | 30.868 | 1.00 | 0.00 | 3A7 |
| ATOM | 3310 | CG | LYS | 453 | 8.486 | −26.409 | 31.399 | 1.00 | 0.00 | 3A7 |
| ATOM | 3311 | CD | LYS | 453 | 8.218 | −27.911 | 31.258 | 1.00 | 0.00 | 3A7 |
| ATOM | 3312 | CE | LYS | 453 | 8.280 | −28.457 | 29.817 | 1.00 | 0.00 | 3A7 |
| ATOM | 3313 | NZ | LYS | 453 | 9.661 | −28.675 | 29.322 | 1.00 | 0.00 | 3A7 |
| ATOM | 3314 | C | LYS | 453 | 10.616 | −24.214 | 32.386 | 1.00 | 0.00 | 3A7 |
| ATOM | 3315 | O | LYS | 453 | 9.869 | −23.577 | 33.112 | 1.00 | 0.00 | 3A7 |
| ATOM | 3316 | N | LEU | 454 | 11.836 | −24.594 | 32.812 | 1.00 | 0.00 | 3A7 |
| ATOM | 3317 | CA | LEU | 454 | 12.304 | −24.349 | 34.154 | 1.00 | 0.00 | 3A7 |
| ATOM | 3318 | CB | LEU | 454 | 13.614 | −25.110 | 34.423 | 1.00 | 0.00 | 3A7 |
| ATOM | 3319 | CG | LEU | 454 | 13.420 | −26.655 | 34.462 | 1.00 | 0.00 | 3A7 |
| ATOM | 3320 | CD1 | LEU | 454 | 14.712 | −27.380 | 34.874 | 1.00 | 0.00 | 3A7 |
| ATOM | 3321 | CD2 | LEU | 454 | 12.265 | −27.101 | 35.377 | 1.00 | 0.00 | 3A7 |
| ATOM | 3322 | C | LEU | 454 | 12.488 | −22.881 | 34.440 | 1.00 | 0.00 | 3A7 |
| ATOM | 3323 | O | LEU | 454 | 12.033 | −22.383 | 35.465 | 1.00 | 0.00 | 3A7 |
| ATOM | 3324 | N | ALA | 455 | 13.101 | −22.129 | 33.501 | 1.00 | 0.00 | 3A7 |
| ATOM | 3325 | CA | ALA | 455 | 13.303 | −20.699 | 33.621 | 1.00 | 0.00 | 3A7 |
| ATOM | 3326 | CB | ALA | 455 | 14.157 | −20.149 | 32.468 | 1.00 | 0.00 | 3A7 |
| ATOM | 3327 | C | ALA | 455 | 12.006 | −19.916 | 33.668 | 1.00 | 0.00 | 3A7 |
| ATOM | 3328 | O | ALA | 455 | 11.803 | −19.112 | 34.569 | 1.00 | 0.00 | 3A7 |
| ATOM | 3329 | N | LEU | 456 | 11.065 | −20.169 | 32.730 | 1.00 | 0.00 | 3A7 |
| ATOM | 3330 | CA | LEU | 456 | 9.795 | −19.468 | 32.642 | 1.00 | 0.00 | 3A7 |
| ATOM | 3331 | CB | LEU | 456 | 9.033 | −19.835 | 31.351 | 1.00 | 0.00 | 3A7 |
| ATOM | 3332 | CG | LEU | 456 | 9.514 | −19.139 | 30.053 | 1.00 | 0.00 | 3A7 |
| ATOM | 3333 | CD1 | LEU | 456 | 10.961 | −18.619 | 30.084 | 1.00 | 0.00 | 3A7 |
| ATOM | 3334 | CD2 | LEU | 456 | 9.277 | −20.054 | 28.836 | 1.00 | 0.00 | 3A7 |
| ATOM | 3335 | C | LEU | 456 | 8.902 | −19.770 | 33.817 | 1.00 | 0.00 | 3A7 |
| ATOM | 3336 | O | LEU | 456 | 8.226 | −18.888 | 34.337 | 1.00 | 0.00 | 3A7 |
| ATOM | 3337 | N | VAL | 457 | 8.933 | −21.025 | 34.323 | 1.00 | 0.00 | 3A7 |
| ATOM | 3338 | CA | VAL | 457 | 8.191 | −21.445 | 35.499 | 1.00 | 0.00 | 3A7 |
| ATOM | 3339 | CB | VAL | 457 | 8.444 | −22.911 | 35.843 | 1.00 | 0.00 | 3A7 |
| ATOM | 3340 | CG1 | VAL | 457 | 8.005 | −23.325 | 37.263 | 1.00 | 0.00 | 3A7 |
| ATOM | 3341 | CG2 | VAL | 457 | 7.670 | −23.765 | 34.841 | 1.00 | 0.00 | 3A7 |
| ATOM | 3342 | C | VAL | 457 | 8.576 | −20.633 | 36.694 | 1.00 | 0.00 | 3A7 |
| ATOM | 3343 | O | VAL | 457 | 7.731 | −20.161 | 37.444 | 1.00 | 0.00 | 3A7 |
| ATOM | 3344 | N | ARG | 458 | 9.892 | −20.443 | 36.892 | 1.00 | 0.00 | 3A7 |
| ATOM | 3345 | CA | ARG | 458 | 10.419 | −19.722 | 38.015 | 1.00 | 0.00 | 3A7 |
| ATOM | 3346 | CB | ARG | 458 | 11.926 | −19.996 | 38.164 | 1.00 | 0.00 | 3A7 |
| ATOM | 3347 | CG | ARG | 458 | 12.246 | −21.369 | 38.758 | 1.00 | 0.00 | 3A7 |
| ATOM | 3348 | CD | ARG | 458 | 13.750 | −21.581 | 38.977 | 1.00 | 0.00 | 3A7 |
| ATOM | 3349 | NE | ARG | 458 | 14.409 | −21.751 | 37.641 | 1.00 | 0.00 | 3A7 |
| ATOM | 3350 | CZ | ARG | 458 | 14.920 | −22.941 | 37.197 | 1.00 | 0.00 | 3A7 |
| ATOM | 3351 | NH1 | ARG | 458 | 15.570 | −22.979 | 35.995 | 1.00 | 0.00 | 3A7 |
| ATOM | 3352 | NH2 | ARG | 458 | 14.813 | −24.080 | 37.937 | 1.00 | 0.00 | 3A7 |
| ATOM | 3353 | C | ARG | 458 | 10.219 | −18.233 | 37.894 | 1.00 | 0.00 | 3A7 |
| ATOM | 3354 | O | ARG | 458 | 9.873 | −17.578 | 38.869 | 1.00 | 0.00 | 3A7 |
| ATOM | 3355 | N | VAL | 459 | 10.398 | −17.640 | 36.691 | 1.00 | 0.00 | 3A7 |
| ATOM | 3356 | CA | VAL | 459 | 10.219 | −16.217 | 36.448 | 1.00 | 0.00 | 3A7 |
| ATOM | 3357 | CB | VAL | 459 | 10.629 | −15.894 | 35.017 | 1.00 | 0.00 | 3A7 |
| ATOM | 3358 | CG1 | VAL | 459 | 10.199 | −14.508 | 34.526 | 1.00 | 0.00 | 3A7 |
| ATOM | 3359 | CG2 | VAL | 459 | 12.165 | −16.014 | 34.950 | 1.00 | 0.00 | 3A7 |
| ATOM | 3360 | C | VAL | 459 | 8.793 | −15.789 | 36.752 | 1.00 | 0.00 | 3A7 |
| ATOM | 3361 | O | VAL | 459 | 8.553 | −14.855 | 37.511 | 1.00 | 0.00 | 3A7 |
| ATOM | 3362 | N | LEU | 460 | 7.804 | −16.538 | 36.226 | 1.00 | 0.00 | 3A7 |
| ATOM | 3363 | CA | LEU | 460 | 6.397 | −16.237 | 36.374 | 1.00 | 0.00 | 3A7 |
| ATOM | 3364 | CB | LEU | 460 | 5.571 | −17.011 | 35.340 | 1.00 | 0.00 | 3A7 |
| ATOM | 3365 | CG | LEU | 460 | 5.883 | −16.588 | 33.893 | 1.00 | 0.00 | 3A7 |
| ATOM | 3366 | CD1 | LEU | 460 | 5.285 | −17.596 | 32.898 | 1.00 | 0.00 | 3A7 |
| ATOM | 3367 | CD2 | LEU | 460 | 5.418 | −15.150 | 33.610 | 1.00 | 0.00 | 3A7 |
| ATOM | 3368 | C | LEU | 460 | 5.857 | −16.582 | 37.736 | 1.00 | 0.00 | 3A7 |
| ATOM | 3369 | O | LEU | 460 | 4.817 | −16.068 | 38.141 | 1.00 | 0.00 | 3A7 |
| ATOM | 3370 | N | GLN | 461 | 6.570 | −17.449 | 38.498 | 1.00 | 0.00 | 3A7 |
| ATOM | 3371 | CA | GLN | 461 | 6.249 | −17.756 | 39.877 | 1.00 | 0.00 | 3A7 |
| ATOM | 3372 | CB | GLN | 461 | 6.961 | −19.025 | 40.414 | 1.00 | 0.00 | 3A7 |
| ATOM | 3373 | CG | GLN | 461 | 6.204 | −19.729 | 41.554 | 1.00 | 0.00 | 3A7 |
| ATOM | 3374 | CD | GLN | 461 | 6.924 | −21.033 | 41.909 | 1.00 | 0.00 | 3A7 |
| ATOM | 3375 | OE1 | GLN | 461 | 7.856 | −21.463 | 41.220 | 1.00 | 0.00 | 3A7 |
| ATOM | 3376 | NE2 | GLN | 461 | 6.464 | −21.670 | 43.029 | 1.00 | 0.00 | 3A7 |
| ATOM | 3377 | C | GLN | 461 | 6.618 | −16.632 | 40.808 | 1.00 | 0.00 | 3A7 |
| ATOM | 3378 | O | GLN | 461 | 5.945 | −16.385 | 41.809 | 1.00 | 0.00 | 3A7 |
| ATOM | 3379 | N | ASN | 462 | 7.741 | −15.950 | 40.487 | 1.00 | 0.00 | 3A7 |
| ATOM | 3380 | CA | ASN | 462 | 8.378 | −15.014 | 41.373 | 1.00 | 0.00 | 3A7 |
| ATOM | 3381 | CB | ASN | 462 | 9.917 | −15.166 | 41.353 | 1.00 | 0.00 | 3A7 |
| ATOM | 3382 | CG | ASN | 462 | 10.321 | −16.436 | 42.117 | 1.00 | 0.00 | 3A7 |
| ATOM | 3383 | OD1 | ASN | 462 | 10.091 | −17.563 | 41.666 | 1.00 | 0.00 | 3A7 |
| ATOM | 3384 | ND2 | ASN | 462 | 10.944 | −16.227 | 43.317 | 1.00 | 0.00 | 3A7 |
| ATOM | 3385 | C | ASN | 462 | 8.026 | −13.582 | 41.041 | 1.00 | 0.00 | 3A7 |
| ATOM | 3386 | O | ASN | 462 | 8.214 | −12.710 | 41.888 | 1.00 | 0.00 | 3A7 |
| ATOM | 3387 | N | PHE | 463 | 7.542 | −13.284 | 39.807 | 1.00 | 0.00 | 3A7 |
| ATOM | 3388 | CA | PHE | 463 | 7.396 | −11.906 | 39.372 | 1.00 | 0.00 | 3A7 |
| ATOM | 3389 | CB | PHE | 463 | 8.606 | −11.416 | 38.535 | 1.00 | 0.00 | 3A7 |
| ATOM | 3390 | CG | PHE | 463 | 9.889 | −11.494 | 39.308 | 1.00 | 0.00 | 3A7 |
| ATOM | 3391 | CD1 | PHE | 463 | 10.834 | −12.474 | 38.996 | 1.00 | 0.00 | 3A7 |
| ATOM | 3392 | CD2 | PHE | 463 | 10.162 | −10.594 | 40.340 | 1.00 | 0.00 | 3A7 |
| ATOM | 3393 | CE1 | PHE | 463 | 12.021 | −12.568 | 39.714 | 1.00 | 0.00 | 3A7 |
| ATOM | 3394 | CE2 | PHE | 463 | 11.352 | −10.684 | 41.057 | 1.00 | 0.00 | 3A7 |
| ATOM | 3395 | CZ | PHE | 463 | 12.280 | −11.673 | 40.747 | 1.00 | 0.00 | 3A7 |
| ATOM | 3396 | C | PHE | 463 | 6.160 | −11.715 | 38.530 | 1.00 | 0.00 | 3A7 |
| ATOM | 3397 | O | PHE | 463 | 5.693 | −12.637 | 37.862 | 1.00 | 0.00 | 3A7 |
| ATOM | 3398 | N | SER | 464 | 5.640 | −10.452 | 38.530 | 1.00 | 0.00 | 3A7 |
| ATOM | 3399 | CA | SER | 464 | 4.607 | −9.957 | 37.639 | 1.00 | 0.00 | 3A7 |
| ATOM | 3400 | CB | SER | 464 | 3.437 | −9.228 | 38.330 | 1.00 | 0.00 | 3A7 |
| ATOM | 3401 | OG | SER | 464 | 3.854 | −8.156 | 39.167 | 1.00 | 0.00 | 3A7 |
| ATOM | 3402 | C | SER | 464 | 5.295 | −9.043 | 36.654 | 1.00 | 0.00 | 3A7 |
| ATOM | 3403 | O | SER | 464 | 5.987 | −8.102 | 37.035 | 1.00 | 0.00 | 3A7 |
| ATOM | 3404 | N | PHE | 465 | 5.122 | −9.324 | 35.343 | 1.00 | 0.00 | 3A7 |
| ATOM | 3405 | CA | PHE | 465 | 5.823 | −8.646 | 34.275 | 1.00 | 0.00 | 3A7 |
| ATOM | 3406 | CB | PHE | 465 | 6.320 | −9.627 | 33.195 | 1.00 | 0.00 | 3A7 |
| ATOM | 3407 | CG | PHE | 465 | 7.476 | −10.312 | 33.835 | 1.00 | 0.00 | 3A7 |
| ATOM | 3408 | CD1 | PHE | 465 | 7.317 | −11.547 | 34.451 | 1.00 | 0.00 | 3A7 |
| ATOM | 3409 | CD2 | PHE | 465 | 8.689 | −9.634 | 33.959 | 1.00 | 0.00 | 3A7 |
| ATOM | 3410 | CE1 | PHE | 465 | 8.353 | −12.080 | 35.204 | 1.00 | 0.00 | 3A7 |
| ATOM | 3411 | CE2 | PHE | 465 | 9.736 | −10.176 | 34.692 | 1.00 | 0.00 | 3A7 |
| ATOM | 3412 | CZ | PHE | 465 | 9.564 | −11.399 | 35.322 | 1.00 | 0.00 | 3A7 |
| ATOM | 3413 | C | PHE | 465 | 4.919 | −7.687 | 33.592 | 1.00 | 0.00 | 3A7 |
| ATOM | 3414 | O | PHE | 465 | 3.724 | −7.936 | 33.425 | 1.00 | 0.00 | 3A7 |
| ATOM | 3415 | N | LYS | 466 | 5.493 | −6.560 | 33.115 | 1.00 | 0.00 | 3A7 |
| ATOM | 3416 | CA | LYS | 466 | 4.734 | −5.582 | 32.382 | 1.00 | 0.00 | 3A7 |
| ATOM | 3417 | CB | LYS | 466 | 4.290 | −4.427 | 33.296 | 1.00 | 0.00 | 3A7 |
| ATOM | 3418 | CG | LYS | 466 | 3.397 | −4.837 | 34.480 | 1.00 | 0.00 | 3A7 |
| ATOM | 3419 | CD | LYS | 466 | 2.972 | −3.643 | 35.348 | 1.00 | 0.00 | 3A7 |
| ATOM | 3420 | CE | LYS | 466 | 2.140 | −4.046 | 36.574 | 1.00 | 0.00 | 3A7 |
| ATOM | 3421 | NZ | LYS | 466 | 2.919 | −4.922 | 37.481 | 1.00 | 0.00 | 3A7 |
| ATOM | 3422 | C | LYS | 466 | 5.602 | −5.043 | 31.290 | 1.00 | 0.00 | 3A7 |
| ATOM | 3423 | O | LYS | 466 | 6.771 | −4.756 | 31.526 | 1.00 | 0.00 | 3A7 |
| ATOM | 3424 | N | PRO | 467 | 5.061 | −4.811 | 30.080 | 1.00 | 0.00 | 3A7 |
| ATOM | 3425 | CA | PRO | 467 | 5.708 | −4.063 | 29.024 | 1.00 | 0.00 | 3A7 |
| ATOM | 3426 | CD | PRO | 467 | 3.897 | −5.529 | 29.577 | 1.00 | 0.00 | 3A7 |
| ATOM | 3427 | CB | PRO | 467 | 4.867 | −4.318 | 27.759 | 1.00 | 0.00 | 3A7 |
| ATOM | 3428 | CG | PRO | 467 | 4.104 | −5.604 | 28.064 | 1.00 | 0.00 | 3A7 |
| ATOM | 3429 | C | PRO | 467 | 5.709 | −2.585 | 29.358 | 1.00 | 0.00 | 3A7 |
| ATOM | 3430 | O | PRO | 467 | 4.657 | −2.040 | 29.696 | 1.00 | 0.00 | 3A7 |
| ATOM | 3431 | N | CYS | 468 | 6.875 | −1.912 | 29.284 | 1.00 | 0.00 | 3A7 |
| ATOM | 3432 | CA | CYS | 468 | 7.012 | −0.524 | 29.684 | 1.00 | 0.00 | 3A7 |
| ATOM | 3433 | CB | CYS | 468 | 8.466 | −0.147 | 30.062 | 1.00 | 0.00 | 3A7 |
| ATOM | 3434 | SG | CYS | 468 | 9.057 | −1.091 | 31.487 | 1.00 | 0.00 | 3A7 |
| ATOM | 3435 | C | CYS | 468 | 6.616 | 0.383 | 28.544 | 1.00 | 0.00 | 3A7 |
| ATOM | 3436 | O | CYS | 468 | 6.411 | 1.583 | 28.713 | 1.00 | 0.00 | 3A7 |
| ATOM | 3437 | N | LYS | 469 | 6.540 | −0.205 | 27.334 | 1.00 | 0.00 | 3A7 |
| ATOM | 3438 | CA | LYS | 469 | 6.355 | 0.499 | 26.104 | 1.00 | 0.00 | 3A7 |
| ATOM | 3439 | CB | LYS | 469 | 7.640 | 0.501 | 25.281 | 1.00 | 0.00 | 3A7 |
| ATOM | 3440 | CG | LYS | 469 | 8.757 | 1.350 | 25.918 | 1.00 | 0.00 | 3A7 |
| ATOM | 3441 | CD | LYS | 469 | 10.000 | 1.530 | 25.028 | 1.00 | 0.00 | 3A7 |
| ATOM | 3442 | CE | LYS | 469 | 9.788 | 2.435 | 23.807 | 1.00 | 0.00 | 3A7 |
| ATOM | 3443 | NZ | LYS | 469 | 9.393 | 3.802 | 24.218 | 1.00 | 0.00 | 3A7 |
| ATOM | 3444 | C | LYS | 469 | 5.347 | −0.291 | 25.354 | 1.00 | 0.00 | 3A7 |
| ATOM | 3445 | O | LYS | 469 | 5.575 | −1.459 | 25.050 | 1.00 | 0.00 | 3A7 |
| ATOM | 3446 | N | GLU | 470 | 4.138 | 0.226 | 25.134 | 1.00 | 0.00 | 3A7 |
| ATOM | 3447 | CA | GLU | 470 | 3.141 | −0.604 | 24.504 | 1.00 | 0.00 | 3A7 |
| ATOM | 3448 | CB | GLU | 470 | 1.701 | −0.299 | 24.997 | 1.00 | 0.00 | 3A7 |
| ATOM | 3449 | CG | GLU | 470 | 1.488 | −0.619 | 26.491 | 1.00 | 0.00 | 3A7 |
| ATOM | 3450 | CD | GLU | 470 | 1.553 | −2.121 | 26.773 | 1.00 | 0.00 | 3A7 |
| ATOM | 3451 | OE1 | GLU | 470 | 1.559 | −2.928 | 25.806 | 1.00 | 0.00 | 3A7 |
| ATOM | 3452 | OE2 | GLU | 470 | 1.584 | −2.480 | 27.981 | 1.00 | 0.00 | 3A7 |
| ATOM | 3453 | C | GLU | 470 | 3.348 | −0.317 | 23.071 | 1.00 | 0.00 | 3A7 |
| ATOM | 3454 | O | GLU | 470 | 4.315 | −0.740 | 22.429 | 1.00 | 0.00 | 3A7 |
| ATOM | 3455 | N | THR | 471 | 2.368 | 0.501 | 22.575 | 1.00 | 0.00 | 3A7 |
| ATOM | 3456 | CA | THR | 471 | 2.160 | 1.284 | 21.360 | 1.00 | 0.00 | 3A7 |
| ATOM | 3457 | CB | THR | 471 | 2.447 | 2.764 | 21.659 | 1.00 | 0.00 | 3A7 |
| ATOM | 3458 | OG1 | THR | 471 | 1.929 | 3.641 | 20.661 | 1.00 | 0.00 | 3A7 |
| ATOM | 3459 | CG2 | THR | 471 | 3.946 | 3.049 | 21.904 | 1.00 | 0.00 | 3A7 |
| ATOM | 3460 | C | THR | 471 | 2.841 | 0.808 | 20.088 | 1.00 | 0.00 | 3A7 |
| ATOM | 3461 | O | THR | 471 | 3.308 | 1.608 | 19.278 | 1.00 | 0.00 | 3A7 |
| ATOM | 3462 | N | GLN | 472 | 2.875 | −0.535 | 19.884 | 1.00 | 0.00 | 3A7 |
| ATOM | 3463 | CA | GLN | 472 | 3.410 | −1.235 | 18.731 | 1.00 | 0.00 | 3A7 |
| ATOM | 3464 | CB | GLN | 472 | 2.623 | −0.915 | 17.428 | 1.00 | 0.00 | 3A7 |
| ATOM | 3465 | CG | GLN | 472 | 2.830 | −1.913 | 16.270 | 1.00 | 0.00 | 3A7 |
| ATOM | 3466 | CD | GLN | 472 | 2.412 | −3.322 | 16.703 | 1.00 | 0.00 | 3A7 |
| ATOM | 3467 | OE1 | GLN | 472 | 3.252 | −4.222 | 16.818 | 1.00 | 0.00 | 3A7 |
| ATOM | 3468 | NE2 | GLN | 472 | 1.077 | −3.502 | 16.944 | 1.00 | 0.00 | 3A7 |
| ATOM | 3469 | C | GLN | 472 | 4.893 | −0.974 | 18.547 | 1.00 | 0.00 | 3A7 |
| ATOM | 3470 | O | GLN | 472 | 5.362 | −0.673 | 17.450 | 1.00 | 0.00 | 3A7 |
| ATOM | 3471 | N | ILE | 473 | 5.672 | −1.090 | 19.658 | 1.00 | 0.00 | 3A7 |
| ATOM | 3472 | CA | ILE | 473 | 7.130 | −0.993 | 19.613 | 1.00 | 0.00 | 3A7 |
| ATOM | 3473 | CB | ILE | 473 | 7.790 | −0.303 | 20.808 | 1.00 | 0.00 | 3A7 |
| ATOM | 3474 | CG2 | ILE | 473 | 9.304 | −0.020 | 20.648 | 1.00 | 0.00 | 3A7 |
| ATOM | 3475 | CG1 | ILE | 473 | 7.030 | 1.009 | 21.146 | 1.00 | 0.00 | 3A7 |
| ATOM | 3476 | CD | ILE | 473 | 7.180 | 2.124 | 20.104 | 1.00 | 0.00 | 3A7 |
| ATOM | 3477 | C | ILE | 473 | 7.840 | −2.153 | 19.174 | 1.00 | 0.00 | 3A7 |
| ATOM | 3478 | O | ILE | 473 | 8.886 | −1.850 | 18.619 | 1.00 | 0.00 | 3A7 |
| ATOM | 3479 | N | PRO | 474 | 7.388 | −3.403 | 19.312 | 1.00 | 0.00 | 3A7 |
| ATOM | 3480 | CA | PRO | 474 | 8.307 | −4.483 | 19.289 | 1.00 | 0.00 | 3A7 |
| ATOM | 3481 | CD | PRO | 474 | 6.293 | −3.751 | 20.216 | 1.00 | 0.00 | 3A7 |
| ATOM | 3482 | CB | PRO | 474 | 7.554 | −5.688 | 19.660 | 1.00 | 0.00 | 3A7 |
| ATOM | 3483 | CG | PRO | 474 | 6.580 | −5.162 | 20.707 | 1.00 | 0.00 | 3A7 |
| ATOM | 3484 | C | PRO | 474 | 9.042 | −4.735 | 18.043 | 1.00 | 0.00 | 3A7 |
| ATOM | 3485 | O | PRO | 474 | 8.461 | −4.845 | 16.967 | 1.00 | 0.00 | 3A7 |
| ATOM | 3486 | N | LEU | 475 | 10.363 | −4.780 | 18.250 | 1.00 | 0.00 | 3A7 |
| ATOM | 3487 | CA | LEU | 475 | 11.281 | −4.868 | 17.212 | 1.00 | 0.00 | 3A7 |
| ATOM | 3488 | CB | LEU | 475 | 12.494 | −3.929 | 17.438 | 1.00 | 0.00 | 3A7 |
| ATOM | 3489 | CG | LEU | 475 | 12.127 | −2.431 | 17.558 | 1.00 | 0.00 | 3A7 |
| ATOM | 3490 | CD1 | LEU | 475 | 13.371 | −1.592 | 17.906 | 1.00 | 0.00 | 3A7 |
| ATOM | 3491 | CD2 | LEU | 475 | 11.432 | −1.891 | 16.293 | 1.00 | 0.00 | 3A7 |
| ATOM | 3492 | C | LEU | 475 | 11.786 | −6.229 | 17.109 | 1.00 | 0.00 | 3A7 |
| ATOM | 3493 | O | LEU | 475 | 12.315 | −6.781 | 18.064 | 1.00 | 0.00 | 3A7 |
| ATOM | 3494 | N | LYS | 476 | 11.627 | −6.792 | 15.915 | 1.00 | 0.00 | 3A7 |
| ATOM | 3495 | CA | LYS | 476 | 12.112 | −8.088 | 15.588 | 1.00 | 0.00 | 3A7 |
| ATOM | 3496 | CB | LYS | 476 | 11.115 | −8.801 | 14.712 | 1.00 | 0.00 | 3A7 |
| ATOM | 3497 | CG | LYS | 476 | 9.772 | −9.009 | 15.441 | 1.00 | 0.00 | 3A7 |
| ATOM | 3498 | CD | LYS | 476 | 8.674 | −9.612 | 14.555 | 1.00 | 0.00 | 3A7 |
| ATOM | 3499 | CE | LYS | 476 | 8.939 | −11.068 | 14.167 | 1.00 | 0.00 | 3A7 |
| ATOM | 3500 | NZ | LYS | 476 | 7.894 | −11.545 | 13.235 | 1.00 | 0.00 | 3A7 |
| ATOM | 3501 | C | LYS | 476 | 13.325 | −7.870 | 14.776 | 1.00 | 0.00 | 3A7 |
| ATOM | 3502 | O | LYS | 476 | 13.337 | −7.042 | 13.874 | 1.00 | 0.00 | 3A7 |
| ATOM | 3503 | N | LEU | 477 | 14.395 | −8.621 | 15.042 | 1.00 | 0.00 | 3A7 |
| ATOM | 3504 | CA | LEU | 477 | 15.554 | −8.551 | 14.210 | 1.00 | 0.00 | 3A7 |
| ATOM | 3505 | CB | LEU | 477 | 16.856 | −8.930 | 14.889 | 1.00 | 0.00 | 3A7 |
| ATOM | 3506 | CG | LEU | 477 | 18.083 | −8.017 | 14.617 | 1.00 | 0.00 | 3A7 |
| ATOM | 3507 | CD1 | LEU | 477 | 19.159 | −8.239 | 15.698 | 1.00 | 0.00 | 3A7 |
| ATOM | 3508 | CD2 | LEU | 477 | 18.705 | −8.182 | 13.223 | 1.00 | 0.00 | 3A7 |
| ATOM | 3509 | C | LEU | 477 | 15.422 | −9.412 | 13.099 | 1.00 | 0.00 | 3A7 |
| ATOM | 3510 | O | LEU | 477 | 14.926 | −10.517 | 13.208 | 1.00 | 0.00 | 3A7 |
| ATOM | 3511 | N | ARG | 478 | 15.810 | −8.885 | 11.967 | 1.00 | 0.00 | 3A7 |
| ATOM | 3512 | CA | ARG | 478 | 15.460 | −9.604 | 10.853 | 1.00 | 0.00 | 3A7 |
| ATOM | 3513 | CB | ARG | 478 | 14.781 | −8.647 | 9.829 | 1.00 | 0.00 | 3A7 |
| ATOM | 3514 | CG | ARG | 478 | 13.512 | −7.955 | 10.358 | 1.00 | 0.00 | 3A7 |
| ATOM | 3515 | CD | ARG | 478 | 12.226 | −8.561 | 9.784 | 1.00 | 0.00 | 3A7 |
| ATOM | 3516 | NE | ARG | 478 | 12.180 | −10.004 | 10.198 | 1.00 | 0.00 | 3A7 |
| ATOM | 3517 | CZ | ARG | 478 | 11.186 | −10.528 | 10.977 | 1.00 | 0.00 | 3A7 |
| ATOM | 3518 | NH1 | ARG | 478 | 11.256 | −11.836 | 11.366 | 1.00 | 0.00 | 3A7 |
| ATOM | 3519 | NH2 | ARG | 478 | 10.121 | −9.764 | 11.352 | 1.00 | 0.00 | 3A7 |
| ATOM | 3520 | C | ARG | 478 | 16.502 | −10.335 | 10.080 | 1.00 | 0.00 | 3A7 |
| ATOM | 3521 | O | ARG | 478 | 16.245 | −10.652 | 8.927 | 1.00 | 0.00 | 3A7 |
| ATOM | 3522 | N | PHE | 479 | 17.697 | −10.610 | 10.632 | 1.00 | 0.00 | 3A7 |
| ATOM | 3523 | CA | PHE | 479 | 18.858 | −10.706 | 9.806 | 1.00 | 0.00 | 3A7 |
| ATOM | 3524 | CB | PHE | 479 | 20.076 | −10.235 | 10.539 | 1.00 | 0.00 | 3A7 |
| ATOM | 3525 | CG | PHE | 479 | 21.147 | −9.680 | 9.633 | 1.00 | 0.00 | 3A7 |
| ATOM | 3526 | CD1 | PHE | 479 | 20.917 | −8.497 | 8.929 | 1.00 | 0.00 | 3A7 |
| ATOM | 3527 | CD2 | PHE | 479 | 22.379 | −10.322 | 9.495 | 1.00 | 0.00 | 3A7 |
| ATOM | 3528 | CE1 | PHE | 479 | 21.894 | −7.970 | 8.093 | 1.00 | 0.00 | 3A7 |
| ATOM | 3529 | CE2 | PHE | 479 | 23.356 | −9.799 | 8.652 | 1.00 | 0.00 | 3A7 |
| ATOM | 3530 | CZ | PHE | 479 | 23.115 | −8.622 | 7.951 | 1.00 | 0.00 | 3A7 |
| ATOM | 3531 | C | PHE | 479 | 19.185 | −12.054 | 9.251 | 1.00 | 0.00 | 3A7 |
| ATOM | 3532 | O | PHE | 479 | 20.077 | −12.120 | 8.413 | 1.00 | 0.00 | 3A7 |
| ATOM | 3533 | N | GLY | 480 | 18.507 | −13.153 | 9.632 | 1.00 | 0.00 | 3A7 |
| ATOM | 3534 | CA | GLY | 480 | 18.751 | −14.416 | 8.969 | 1.00 | 0.00 | 3A7 |
| ATOM | 3535 | C | GLY | 480 | 19.990 | −15.080 | 9.499 | 1.00 | 0.00 | 3A7 |
| ATOM | 3536 | O | GLY | 480 | 21.012 | −14.455 | 9.781 | 1.00 | 0.00 | 3A7 |
| ATOM | 3537 | N | GLY | 481 | 19.874 | −16.400 | 9.691 | 1.00 | 0.00 | 3A7 |
| ATOM | 3538 | CA | GLY | 481 | 20.747 | −17.199 | 10.480 | 1.00 | 0.00 | 3A7 |
| ATOM | 3539 | C | GLY | 481 | 19.999 | −17.404 | 11.756 | 1.00 | 0.00 | 3A7 |
| ATOM | 3540 | O | GLY | 481 | 19.686 | −18.530 | 12.126 | 1.00 | 0.00 | 3A7 |
| ATOM | 3541 | N | LEU | 482 | 19.687 | −16.297 | 12.465 | 1.00 | 0.00 | 3A7 |
| ATOM | 3542 | CA | LEU | 482 | 19.157 | −16.393 | 13.801 | 1.00 | 0.00 | 3A7 |
| ATOM | 3543 | CB | LEU | 482 | 20.314 | −16.416 | 14.840 | 1.00 | 0.00 | 3A7 |
| ATOM | 3544 | CG | LEU | 482 | 20.009 | −17.132 | 16.175 | 1.00 | 0.00 | 3A7 |
| ATOM | 3545 | CD1 | LEU | 482 | 18.926 | −16.443 | 17.020 | 1.00 | 0.00 | 3A7 |
| ATOM | 3546 | CD2 | LEU | 482 | 19.738 | −18.634 | 15.963 | 1.00 | 0.00 | 3A7 |
| ATOM | 3547 | C | LEU | 482 | 18.293 | −15.192 | 13.998 | 1.00 | 0.00 | 3A7 |
| ATOM | 3548 | O | LEU | 482 | 18.787 | −14.069 | 13.996 | 1.00 | 0.00 | 3A7 |
| ATOM | 3549 | N | LEU | 483 | 16.972 | −15.412 | 14.210 | 1.00 | 0.00 | 3A7 |
| ATOM | 3550 | CA | LEU | 483 | 16.003 | −14.371 | 14.462 | 1.00 | 0.00 | 3A7 |
| ATOM | 3551 | CB | LEU | 483 | 14.571 | −14.799 | 14.069 | 1.00 | 0.00 | 3A7 |
| ATOM | 3552 | CG | LEU | 483 | 14.356 | −14.905 | 12.543 | 1.00 | 0.00 | 3A7 |
| ATOM | 3553 | CD1 | LEU | 483 | 12.892 | −15.253 | 12.231 | 1.00 | 0.00 | 3A7 |
| ATOM | 3554 | CD2 | LEU | 483 | 14.772 | −13.634 | 11.775 | 1.00 | 0.00 | 3A7 |
| ATOM | 3555 | C | LEU | 483 | 15.995 | −14.055 | 15.923 | 1.00 | 0.00 | 3A7 |
| ATOM | 3556 | O | LEU | 483 | 15.799 | −14.926 | 16.767 | 1.00 | 0.00 | 3A7 |
| ATOM | 3557 | N | LEU | 484 | 16.246 | −12.778 | 16.236 | 1.00 | 0.00 | 3A7 |
| ATOM | 3558 | CA | LEU | 484 | 16.374 | −12.310 | 17.588 | 1.00 | 0.00 | 3A7 |
| ATOM | 3559 | CB | LEU | 484 | 17.753 | −11.620 | 17.812 | 1.00 | 0.00 | 3A7 |
| ATOM | 3560 | CG | LEU | 484 | 18.984 | −12.548 | 17.710 | 1.00 | 0.00 | 3A7 |
| ATOM | 3561 | CD1 | LEU | 484 | 20.271 | −11.745 | 17.445 | 1.00 | 0.00 | 3A7 |
| ATOM | 3562 | CD2 | LEU | 484 | 19.140 | −13.413 | 18.970 | 1.00 | 0.00 | 3A7 |
| ATOM | 3563 | C | LEU | 484 | 15.345 | −11.234 | 17.861 | 1.00 | 0.00 | 3A7 |
| ATOM | 3564 | O | LEU | 484 | 14.779 | −10.598 | 16.981 | 1.00 | 0.00 | 3A7 |
| ATOM | 3565 | N | THR | 485 | 15.149 | −10.896 | 19.142 | 1.00 | 0.00 | 3A7 |
| ATOM | 3566 | CA | THR | 485 | 14.449 | −9.702 | 19.526 | 1.00 | 0.00 | 3A7 |
| ATOM | 3567 | CB | THR | 485 | 13.528 | −9.902 | 20.697 | 1.00 | 0.00 | 3A7 |
| ATOM | 3568 | OG1 | THR | 485 | 12.643 | −10.981 | 20.430 | 1.00 | 0.00 | 3A7 |
| ATOM | 3569 | CG2 | THR | 485 | 12.694 | −8.634 | 20.978 | 1.00 | 0.00 | 3A7 |
| ATOM | 3570 | C | THR | 485 | 15.542 | −8.739 | 19.870 | 1.00 | 0.00 | 3A7 |
| ATOM | 3571 | O | THR | 485 | 16.525 | −9.095 | 20.514 | 1.00 | 0.00 | 3A7 |
| ATOM | 3572 | N | GLU | 486 | 15.407 | −7.491 | 19.395 | 1.00 | 0.00 | 3A7 |
| ATOM | 3573 | CA | GLU | 486 | 16.478 | −6.537 | 19.402 | 1.00 | 0.00 | 3A7 |
| ATOM | 3574 | CB | GLU | 486 | 16.229 | −5.476 | 18.329 | 1.00 | 0.00 | 3A7 |
| ATOM | 3575 | CG | GLU | 486 | 16.097 | −6.056 | 16.918 | 1.00 | 0.00 | 3A7 |
| ATOM | 3576 | CD | GLU | 486 | 16.045 | −4.920 | 15.900 | 1.00 | 0.00 | 3A7 |
| ATOM | 3577 | OE1 | GLU | 486 | 15.044 | −4.847 | 15.140 | 1.00 | 0.00 | 3A7 |
| ATOM | 3578 | OE2 | GLU | 486 | 17.014 | −4.115 | 15.863 | 1.00 | 0.00 | 3A7 |
| ATOM | 3579 | C | GLU | 486 | 16.763 | −5.904 | 20.729 | 1.00 | 0.00 | 3A7 |
| ATOM | 3580 | O | GLU | 486 | 17.821 | −6.121 | 21.297 | 1.00 | 0.00 | 3A7 |
| ATOM | 3581 | N | LYS | 487 | 15.871 | −5.074 | 21.275 | 1.00 | 0.00 | 3A7 |
| ATOM | 3582 | CA | LYS | 487 | 16.181 | −4.394 | 22.523 | 1.00 | 0.00 | 3A7 |
| ATOM | 3583 | CB | LYS | 487 | 16.681 | −2.935 | 22.345 | 1.00 | 0.00 | 3A7 |
| ATOM | 3584 | CG | LYS | 487 | 18.078 | −2.831 | 21.710 | 1.00 | 0.00 | 3A7 |
| ATOM | 3585 | CD | LYS | 487 | 18.582 | −1.386 | 21.553 | 1.00 | 0.00 | 3A7 |
| ATOM | 3586 | CE | LYS | 487 | 17.737 | −0.521 | 20.606 | 1.00 | 0.00 | 3A7 |
| ATOM | 3587 | NZ | LYS | 487 | 17.683 | −1.111 | 19.249 | 1.00 | 0.00 | 3A7 |
| ATOM | 3588 | C | LYS | 487 | 14.847 | −4.380 | 23.208 | 1.00 | 0.00 | 3A7 |
| ATOM | 3589 | O | LYS | 487 | 14.110 | −3.436 | 22.965 | 1.00 | 0.00 | 3A7 |
| ATOM | 3590 | N | PRO | 488 | 14.469 | −5.369 | 24.036 | 1.00 | 0.00 | 3A7 |
| ATOM | 3591 | CA | PRO | 488 | 13.160 | −5.418 | 24.666 | 1.00 | 0.00 | 3A7 |
| ATOM | 3592 | CD | PRO | 488 | 15.099 | −6.687 | 23.995 | 1.00 | 0.00 | 3A7 |
| ATOM | 3593 | CB | PRO | 488 | 12.772 | −6.900 | 24.541 | 1.00 | 0.00 | 3A7 |
| ATOM | 3594 | CG | PRO | 488 | 14.103 | −7.653 | 24.646 | 1.00 | 0.00 | 3A7 |
| ATOM | 3595 | C | PRO | 488 | 13.285 | −4.931 | 26.063 | 1.00 | 0.00 | 3A7 |
| ATOM | 3596 | O | PRO | 488 | 14.207 | −5.350 | 26.760 | 1.00 | 0.00 | 3A7 |
| ATOM | 3597 | N | ILE | 489 | 12.369 | −4.042 | 26.506 | 1.00 | 0.00 | 3A7 |
| ATOM | 3598 | CA | ILE | 489 | 12.431 | −3.564 | 27.858 | 1.00 | 0.00 | 3A7 |
| ATOM | 3599 | CB | ILE | 489 | 12.824 | −2.096 | 28.021 | 1.00 | 0.00 | 3A7 |
| ATOM | 3600 | CG2 | ILE | 489 | 14.258 | −1.923 | 27.475 | 1.00 | 0.00 | 3A7 |
| ATOM | 3601 | CG1 | ILE | 489 | 11.831 | −1.092 | 27.392 | 1.00 | 0.00 | 3A7 |
| ATOM | 3602 | CD | ILE | 489 | 12.182 | 0.356 | 27.747 | 1.00 | 0.00 | 3A7 |
| ATOM | 3603 | C | ILE | 489 | 11.136 | −3.837 | 28.577 | 1.00 | 0.00 | 3A7 |
| ATOM | 3604 | O | ILE | 489 | 10.052 | −3.733 | 28.009 | 1.00 | 0.00 | 3A7 |
| ATOM | 3605 | N | VAL | 490 | 11.230 | −4.209 | 29.878 | 1.00 | 0.00 | 3A7 |
| ATOM | 3606 | CA | VAL | 490 | 10.101 | −4.648 | 30.674 | 1.00 | 0.00 | 3A7 |
| ATOM | 3607 | CB | VAL | 490 | 9.925 | −6.155 | 30.572 | 1.00 | 0.00 | 3A7 |
| ATOM | 3608 | CG1 | VAL | 490 | 9.245 | −6.911 | 31.734 | 1.00 | 0.00 | 3A7 |
| ATOM | 3609 | CG2 | VAL | 490 | 9.143 | −6.483 | 29.279 | 1.00 | 0.00 | 3A7 |
| ATOM | 3610 | C | VAL | 490 | 10.398 | −4.303 | 32.085 | 1.00 | 0.00 | 3A7 |
| ATOM | 3611 | O | VAL | 490 | 11.537 | −4.064 | 32.455 | 1.00 | 0.00 | 3A7 |
| ATOM | 3612 | N | LEU | 491 | 9.344 | −4.289 | 32.927 | 1.00 | 0.00 | 3A7 |
| ATOM | 3613 | CA | LEU | 491 | 9.438 | −4.005 | 34.328 | 1.00 | 0.00 | 3A7 |
| ATOM | 3614 | CB | LEU | 491 | 8.379 | −2.956 | 34.702 | 1.00 | 0.00 | 3A7 |
| ATOM | 3615 | CG | LEU | 491 | 8.991 | −1.575 | 35.041 | 1.00 | 0.00 | 3A7 |
| ATOM | 3616 | CD1 | LEU | 491 | 7.898 | −0.495 | 35.142 | 1.00 | 0.00 | 3A7 |
| ATOM | 3617 | CD2 | LEU | 491 | 9.848 | −1.615 | 36.322 | 1.00 | 0.00 | 3A7 |
| ATOM | 3618 | C | LEU | 491 | 9.183 | −5.273 | 35.064 | 1.00 | 0.00 | 3A7 |
| ATOM | 3619 | O | LEU | 491 | 8.303 | −6.043 | 34.680 | 1.00 | 0.00 | 3A7 |
| ATOM | 3620 | N | LYS | 492 | 9.934 | −5.530 | 36.165 | 1.00 | 0.00 | 3A7 |
| ATOM | 3621 | CA | LYS | 492 | 9.855 | −6.761 | 36.930 | 1.00 | 0.00 | 3A7 |
| ATOM | 3622 | CB | LYS | 492 | 11.254 | −7.391 | 37.203 | 1.00 | 0.00 | 3A7 |
| ATOM | 3623 | CG | LYS | 492 | 12.184 | −6.661 | 38.201 | 1.00 | 0.00 | 3A7 |
| ATOM | 3624 | CD | LYS | 492 | 12.735 | −5.301 | 37.743 | 1.00 | 0.00 | 3A7 |
| ATOM | 3625 | CE | LYS | 492 | 12.568 | −4.191 | 38.784 | 1.00 | 0.00 | 3A7 |
| ATOM | 3626 | NZ | LYS | 492 | 11.143 | −3.831 | 38.925 | 1.00 | 0.00 | 3A7 |
| ATOM | 3627 | C | LYS | 492 | 9.148 | −6.537 | 38.247 | 1.00 | 0.00 | 3A7 |
| ATOM | 3628 | O | LYS | 492 | 9.344 | −7.295 | 39.196 | 1.00 | 0.00 | 3A7 |
| ATOM | 3629 | N | ALA | 493 | 8.315 | −5.466 | 38.338 | 1.00 | 0.00 | 3A7 |
| ATOM | 3630 | CA | ALA | 493 | 7.783 | −4.923 | 39.576 | 1.00 | 0.00 | 3A7 |
| ATOM | 3631 | CB | ALA | 493 | 7.164 | −3.529 | 39.347 | 1.00 | 0.00 | 3A7 |
| ATOM | 3632 | C | ALA | 493 | 6.735 | −5.798 | 40.223 | 1.00 | 0.00 | 3A7 |
| ATOM | 3633 | O | ALA | 493 | 5.607 | −5.896 | 39.745 | 1.00 | 0.00 | 3A7 |
| ATOM | 3634 | N | GLU | 494 | 7.121 | −6.449 | 41.348 | 1.00 | 0.00 | 3A7 |
| ATOM | 3635 | CA | GLU | 494 | 6.270 | −7.353 | 42.081 | 1.00 | 0.00 | 3A7 |
| ATOM | 3636 | CB | GLU | 494 | 6.221 | −8.771 | 41.447 | 1.00 | 0.00 | 3A7 |
| ATOM | 3637 | CG | GLU | 494 | 5.073 | −9.691 | 41.921 | 1.00 | 0.00 | 3A7 |
| ATOM | 3638 | CD | GLU | 494 | 5.351 | −10.327 | 43.282 | 1.00 | 0.00 | 3A7 |
| ATOM | 3639 | OE1 | GLU | 494 | 6.416 | −10.984 | 43.425 | 1.00 | 0.00 | 3A7 |
| ATOM | 3640 | OE2 | GLU | 494 | 4.495 | −10.171 | 44.193 | 1.00 | 0.00 | 3A7 |
| ATOM | 3641 | C | GLU | 494 | 6.833 | −7.451 | 43.468 | 1.00 | 0.00 | 3A7 |
| ATOM | 3642 | O | GLU | 494 | 6.097 | −7.692 | 44.424 | 1.00 | 0.00 | 3A7 |
| ATOM | 3643 | N | SER | 495 | 8.180 | −7.291 | 43.592 | 1.00 | 0.00 | 3A7 |
| ATOM | 3644 | CA | SER | 495 | 9.005 | −7.634 | 44.741 | 1.00 | 0.00 | 3A7 |
| ATOM | 3645 | CB | SER | 495 | 10.488 | −7.267 | 44.500 | 1.00 | 0.00 | 3A7 |
| ATOM | 3646 | OG | SER | 495 | 10.963 | −7.890 | 43.315 | 1.00 | 0.00 | 3A7 |
| ATOM | 3647 | C | SER | 495 | 8.584 | −6.990 | 46.046 | 1.00 | 0.00 | 3A7 |
| ATOM | 3648 | O | SER | 495 | 8.318 | −5.791 | 46.117 | 1.00 | 0.00 | 3A7 |
| ATOM | 3649 | N | ARG | 496 | 8.506 | −7.831 | 47.102 | 1.00 | 0.00 | 3A7 |
| ATOM | 3650 | CA | ARG | 496 | 8.050 | −7.468 | 48.419 | 1.00 | 0.00 | 3A7 |
| ATOM | 3651 | CB | ARG | 496 | 6.781 | −8.270 | 48.810 | 1.00 | 0.00 | 3A7 |
| ATOM | 3652 | CG | ARG | 496 | 6.173 | −7.918 | 50.178 | 1.00 | 0.00 | 3A7 |
| ATOM | 3653 | CD | ARG | 496 | 4.896 | −8.715 | 50.471 | 1.00 | 0.00 | 3A7 |
| ATOM | 3654 | NE | ARG | 496 | 4.413 | −8.345 | 51.841 | 1.00 | 0.00 | 3A7 |
| ATOM | 3655 | CZ | ARG | 496 | 3.354 | −8.981 | 52.430 | 1.00 | 0.00 | 3A7 |
| ATOM | 3656 | NH1 | ARG | 496 | 2.955 | −8.609 | 53.681 | 1.00 | 0.00 | 3A7 |
| ATOM | 3657 | NH2 | ARG | 496 | 2.696 | −9.982 | 51.778 | 1.00 | 0.00 | 3A7 |
| ATOM | 3658 | C | ARG | 496 | 9.179 | −7.785 | 49.356 | 1.00 | 0.00 | 3A7 |
| ATOM | 3659 | O | ARG | 496 | 9.927 | −8.738 | 49.139 | 1.00 | 0.00 | 3A7 |
| ATOM | 3660 | N | ASP | 497 | 9.312 | −6.984 | 50.445 | 1.00 | 0.00 | 3A7 |
| ATOM | 3661 | CA | ASP | 497 | 10.298 | −7.171 | 51.489 | 1.00 | 0.00 | 3A7 |
| ATOM | 3662 | CB | ASP | 497 | 10.821 | −5.830 | 52.089 | 1.00 | 0.00 | 3A7 |
| ATOM | 3663 | CG | ASP | 497 | 9.707 | −4.881 | 52.551 | 1.00 | 0.00 | 3A7 |
| ATOM | 3664 | OD1 | ASP | 497 | 8.940 | −4.387 | 51.682 | 1.00 | 0.00 | 3A7 |
| ATOM | 3665 | OD2 | ASP | 497 | 9.625 | −4.629 | 53.783 | 1.00 | 0.00 | 3A7 |
| ATOM | 3666 | C | ASP | 497 | 9.711 | −8.068 | 52.560 | 1.00 | 0.00 | 3A7 |
| ATOM | 3667 | O | ASP | 497 | 8.985 | −7.622 | 53.447 | 1.00 | 0.00 | 3A7 |
| ATOM | 3668 | N | GLU | 498 | 10.021 | −9.384 | 52.458 | 1.00 | 0.00 | 3A7 |
| ATOM | 3669 | CA | GLU | 498 | 9.495 | −10.423 | 53.314 | 1.00 | 0.00 | 3A7 |
| ATOM | 3670 | CB | GLU | 498 | 8.919 | −11.610 | 52.501 | 1.00 | 0.00 | 3A7 |
| ATOM | 3671 | CG | GLU | 498 | 7.782 | −11.185 | 51.557 | 1.00 | 0.00 | 3A7 |
| ATOM | 3672 | CD | GLU | 498 | 7.253 | −12.413 | 50.822 | 1.00 | 0.00 | 3A7 |
| ATOM | 3673 | OE1 | GLU | 498 | 6.045 | −12.728 | 50.989 | 1.00 | 0.00 | 3A7 |
| ATOM | 3674 | OE2 | GLU | 498 | 8.050 | −13.049 | 50.081 | 1.00 | 0.00 | 3A7 |
| ATOM | 3675 | C | GLU | 498 | 10.610 | −10.918 | 54.195 | 1.00 | 0.00 | 3A7 |
| ATOM | 3676 | O | GLU | 498 | 11.726 | −10.403 | 54.157 | 1.00 | 0.00 | 3A7 |
| ATOM | 3677 | N | THR | 499 | 10.311 | −11.961 | 55.010 | 1.00 | 0.00 | 3A7 |
| ATOM | 3678 | CA | THR | 499 | 11.248 | −12.606 | 55.905 | 1.00 | 0.00 | 3A7 |
| ATOM | 3679 | CB | THR | 499 | 10.615 | −12.970 | 57.245 | 1.00 | 0.00 | 3A7 |
| ATOM | 3680 | OG1 | THR | 499 | 9.418 | −13.726 | 57.084 | 1.00 | 0.00 | 3A7 |
| ATOM | 3681 | CG2 | THR | 499 | 10.291 | −11.664 | 57.998 | 1.00 | 0.00 | 3A7 |
| ATOM | 3682 | C | THR | 499 | 11.798 | −13.836 | 55.217 | 1.00 | 0.00 | 3A7 |
| ATOM | 3683 | O | THR | 499 | 11.311 | −14.244 | 54.163 | 1.00 | 0.00 | 3A7 |
| ATOM | 3684 | N | VAL | 500 | 12.842 | −14.455 | 55.826 | 1.00 | 0.00 | 3A7 |
| ATOM | 3685 | CA | VAL | 500 | 13.521 | −15.621 | 55.300 | 1.00 | 0.00 | 3A7 |
| ATOM | 3686 | CB | VAL | 500 | 15.021 | −15.588 | 55.594 | 1.00 | 0.00 | 3A7 |
| ATOM | 3687 | CG1 | VAL | 500 | 15.312 | −15.508 | 57.109 | 1.00 | 0.00 | 3A7 |
| ATOM | 3688 | CG2 | VAL | 500 | 15.739 | −16.768 | 54.903 | 1.00 | 0.00 | 3A7 |
| ATOM | 3689 | C | VAL | 500 | 12.857 | −16.864 | 55.847 | 1.00 | 0.00 | 3A7 |
| ATOM | 3690 | O | VAL | 500 | 12.610 | −16.984 | 57.047 | 1.00 | 0.00 | 3A7 |
| ATOM | 3691 | N | SER | 501 | 12.542 | −17.816 | 54.939 | 1.00 | 0.00 | 3A7 |
| ATOM | 3692 | CA | SER | 501 | 11.904 | −19.059 | 55.286 | 1.00 | 0.00 | 3A7 |
| ATOM | 3693 | CB | SER | 501 | 10.354 | −18.950 | 55.320 | 1.00 | 0.00 | 3A7 |
| ATOM | 3694 | OG | SER | 501 | 9.744 | −20.142 | 55.806 | 1.00 | 0.00 | 3A7 |
| ATOM | 3695 | C | SER | 501 | 12.356 | −20.035 | 54.235 | 1.00 | 0.00 | 3A7 |
| ATOM | 3696 | O | SER | 501 | 12.898 | −21.094 | 54.550 | 1.00 | 0.00 | 3A7 |
| ATOM | 3697 | N | GLY | 502 | 12.135 | −19.677 | 52.947 | 1.00 | 0.00 | 3A7 |
| ATOM | 3698 | CA | GLY | 502 | 12.533 | −20.464 | 51.805 | 1.00 | 0.00 | 3A7 |
| ATOM | 3699 | C | GLY | 502 | 13.587 | −19.693 | 51.074 | 1.00 | 0.00 | 3A7 |
| ATOM | 3700 | O | GLY | 502 | 13.335 | −18.585 | 50.602 | 1.00 | 0.00 | 3A7 |
| ATOM | 3701 | N | ALA | 503 | 14.804 | −20.283 | 50.980 | 1.00 | 0.00 | 3A7 |
| ATOM | 3702 | CA | ALA | 503 | 15.970 | −19.715 | 50.338 | 1.00 | 0.00 | 3A7 |
| ATOM | 3703 | CB | ALA | 503 | 15.775 | −19.401 | 48.838 | 1.00 | 0.00 | 3A7 |
| ATOM | 3704 | C | ALA | 503 | 16.423 | −18.443 | 51.073 | 1.00 | 0.00 | 3A7 |
| ATOM | 3705 | OT1 | ALA | 503 | 17.016 | −18.589 | 52.175 | 1.00 | 0.00 | 3A7 |
| ATOM | 3706 | OT2 | ALA | 503 | 16.167 | −17.323 | 50.557 | 1.00 | 0.00 | 3A7 |
| TER | 3707 | | ALA | 503 | | | | | | |
| HETATM | 3708 | FE | HEM | 600 | 19.802 | −26.909 | 18.195 | 1.00 | 0.00 | HEM |
| HETATM | 3709 | NA | HEM | 600 | 19.117 | −26.973 | 16.343 | 1.00 | 0.00 | HEM |
| HETATM | 3710 | NB | HEM | 600 | 18.291 | −25.763 | 18.746 | 1.00 | 0.00 | HEM |
| HETATM | 3711 | NC | HEM | 600 | 20.542 | −26.830 | 20.017 | 1.00 | 0.00 | HEM |
| HETATM | 3712 | ND | HEM | 600 | 21.332 | −28.034 | 17.616 | 1.00 | 0.00 | HEM |
| HETATM | 3713 | C1A | HEM | 600 | 19.627 | −27.653 | 15.248 | 1.00 | 0.00 | HEM |
| HETATM | 3714 | C2A | HEM | 600 | 18.787 | −27.461 | 14.072 | 1.00 | 0.00 | HEM |
| HETATM | 3715 | C3A | HEM | 600 | 17.812 | −26.605 | 14.439 | 1.00 | 0.00 | HEM |
| HETATM | 3716 | C4A | HEM | 600 | 17.993 | −26.338 | 15.849 | 1.00 | 0.00 | HEM |
| HETATM | 3717 | C1B | HEM | 600 | 17.258 | −25.288 | 17.964 | 1.00 | 0.00 | HEM |
| HETATM | 3718 | C2B | HEM | 600 | 16.334 | −24.465 | 18.724 | 1.00 | 0.00 | HEM |
| HETATM | 3719 | C3B | HEM | 600 | 16.805 | −24.471 | 20.029 | 1.00 | 0.00 | HEM |
| HETATM | 3720 | C4B | HEM | 600 | 18.026 | −25.267 | 20.000 | 1.00 | 0.00 | HEM |
| HETATM | 3721 | C1C | HEM | 600 | 20.002 | −26.220 | 21.122 | 1.00 | 0.00 | HEM |
| HETATM | 3722 | C2C | HEM | 600 | 20.837 | −26.398 | 22.303 | 1.00 | 0.00 | HEM |
| HETATM | 3723 | C3C | HEM | 600 | 21.948 | −27.105 | 21.896 | 1.00 | 0.00 | HEM |
| HETATM | 3724 | C4C | HEM | 600 | 21.734 | −27.359 | 20.473 | 1.00 | 0.00 | HEM |
| HETATM | 3725 | C1D | HEM | 600 | 22.427 | −28.419 | 18.358 | 1.00 | 0.00 | HEM |
| HETATM | 3726 | C2D | HEM | 600 | 23.355 | −29.229 | 17.581 | 1.00 | 0.00 | HEM |
| HETATM | 3727 | C3D | HEM | 600 | 22.815 | −29.362 | 16.350 | 1.00 | 0.00 | HEM |
| HETATM | 3728 | C4D | HEM | 600 | 21.589 | −28.591 | 16.366 | 1.00 | 0.00 | HEM |
| HETATM | 3729 | CHA | HEM | 600 | 20.801 | −28.392 | 15.257 | 1.00 | 0.00 | HEM |
| HETATM | 3730 | CHB | HEM | 600 | 17.121 | −25.582 | 16.615 | 1.00 | 0.00 | HEM |
| HETATM | 3731 | CHC | HEM | 600 | 18.841 | −25.488 | 21.097 | 1.00 | 0.00 | HEM |
| HETATM | 3732 | CHD | HEM | 600 | 22.615 | −28.078 | 19.676 | 1.00 | 0.00 | HEM |
| HETATM | 3733 | CMA | HEM | 600 | 16.761 | −26.027 | 13.539 | 1.00 | 0.00 | HEM |
| HETATM | 3734 | CAA | HEM | 600 | 18.874 | −28.152 | 12.738 | 1.00 | 0.00 | HEM |
| HETATM | 3735 | CBA | HEM | 600 | 19.655 | −27.341 | 11.731 | 1.00 | 0.00 | HEM |
| HETATM | 3736 | CGA | HEM | 600 | 19.868 | −28.082 | 10.424 | 1.00 | 0.00 | HEM |
| HETATM | 3737 | O1A | HEM | 600 | 18.997 | −28.897 | 10.031 | 1.00 | 0.00 | HEM |
| HETATM | 3738 | O2A | HEM | 600 | 20.918 | −27.812 | 9.777 | 1.00 | 0.00 | HEM |
| HETATM | 3739 | CMB | HEM | 600 | 15.130 | −23.749 | 18.107 | 1.00 | 0.00 | HEM |
| HETATM | 3740 | CAB | HEM | 600 | 16.311 | −23.874 | 21.208 | 1.00 | 0.00 | HEM |
| HETATM | 3741 | CBB | HEM | 600 | 15.206 | −23.138 | 21.387 | 1.00 | 0.00 | HEM |
| HETATM | 3742 | CMC | HEM | 600 | 20.479 | −25.906 | 23.689 | 1.00 | 0.00 | HEM |
| HETATM | 3743 | CAC | HEM | 600 | 23.094 | −27.556 | 22.589 | 1.00 | 0.00 | HEM |
| HETATM | 3744 | CBC | HEM | 600 | 23.305 | −27.610 | 23.914 | 1.00 | 0.00 | HEM |
| HETATM | 3745 | CMD | HEM | 600 | 24.679 | −29.779 | 18.033 | 1.00 | 0.00 | HEM |
| HETATM | 3746 | CAD | HEM | 600 | 23.358 | −30.183 | 15.203 | 1.00 | 0.00 | HEM |
| HETATM | 3747 | CBD | HEM | 600 | 22.726 | −31.591 | 15.127 | 1.00 | 0.00 | HEM |
| HETATM | 3748 | CGD | HEM | 600 | 23.332 | −32.470 | 14.027 | 1.00 | 0.00 | HEM |
| HETATM | 3749 | O1D | HEM | 600 | 23.719 | −33.625 | 14.347 | 1.00 | 0.00 | HEM |
| HETATM | 3750 | O2D | HEM | 600 | 23.390 | −32.020 | 12.856 | 1.00 | 0.00 | HEM |
| END | | | | | | | | | | |

Sequences:

SEQ ID N^{o}1: P450 Nor, crystal structure 1 rom

SEQ ID N^{o}2: P450 Ery F, crystal structure 1 oxa

SEQ ID N^{o}3: P450 Terp, crystal structure 1 cpt

SEQ ID N^{o}4: P450 Cam, crystal structure 3cpp

SEQ ID N^{o}5: P450 BM3, crystal structure 2hpd

The sequence corresponding to the PDB structure includes 471 residues. For more clarity in FIG. 1, the last 12 residues have been omitted, the C-terminal part having no equivalent counterpart in the other structures aligned.

SEQ ID N^{o}6: P450 2C5, crystal structure 1dt6

Cyp2C5 from Oryctolagus cuniculus (Rabbit), with membrane spanning residues 3-21 deleted and a 4 residue histidine tag at the C-Terminus containing additional internal mutations.

SEQ ID N^{o}7: P450 2C5 rabbit

Sequence corresponding to the non-mutated CYP 2C5 gene from Oryctolagus cuniculus (Rabbit), consistently with SwissProt CPC5_RABIT P00179.

SEQ ID N^{o} 8: CYP51, crystal structure 1e9x

Cyp51 from Mycobacterium tuberculosis, with a 4 residue histidine tag at the C-Terminus.

SEQ ID N^{o}9: CYP3A1 rat

SEQ ID N^{o}10: CYP3A3 human

Cytochrome P-450, a possible variant of CYP3A4, inducible by glucocorticoids in human liver.

SEQ ID N^{o}11: CYP3A4 human

Numbering starts at Ala 1 (first residue Met is not included, consistently with SwissProt CP34_HUMAN P08684)

SEQ ID N^{o}12: CYP3A5 human

SEQ ID N^{o}13: CYP3A43 human

SEQ ID N^{o}14: CYP3A6 rabbit

SEQ ID N^{o}15: CYP3A7 human

SEQ ID N^{o}16: CYP3A12 dog

SEQ ID N^{o}17: CYP3A29 pig

SEQ ID N^{o}18: CYP3A13 mouse

FIG. 1: Structure-based alignment of human cytochromes P450 3A3, 3A4, 3A5, 3A7 and 3A43 and of selected mammalian P450 3A isozymes, with bacterial P450 crystal template structures and rabbit P450 2C5 crystal template structure.

Sequence numbering is indicated for each enzyme of the structural template and for the human 3A4 and 3A7 isozymes, as examples given in the present invention. This alignment is first based on the structural alignment of bacterial P450s and rabbit P450 2C5 derived from GOK analysis. Human P450 3A sequences were then aligned with in-house tools that locates the CSBs on the target sequence. The alignment shown outside the CSBs is not relevant, as there is no structural information available in these regions. The CSB sequences are indicated by bold uppercase characters and are highlighted in grey. Amino acids strictly conserved between CYP3A and 2C5, or between CYP3A and all the sequences of crystal structures, are highlighted in black.

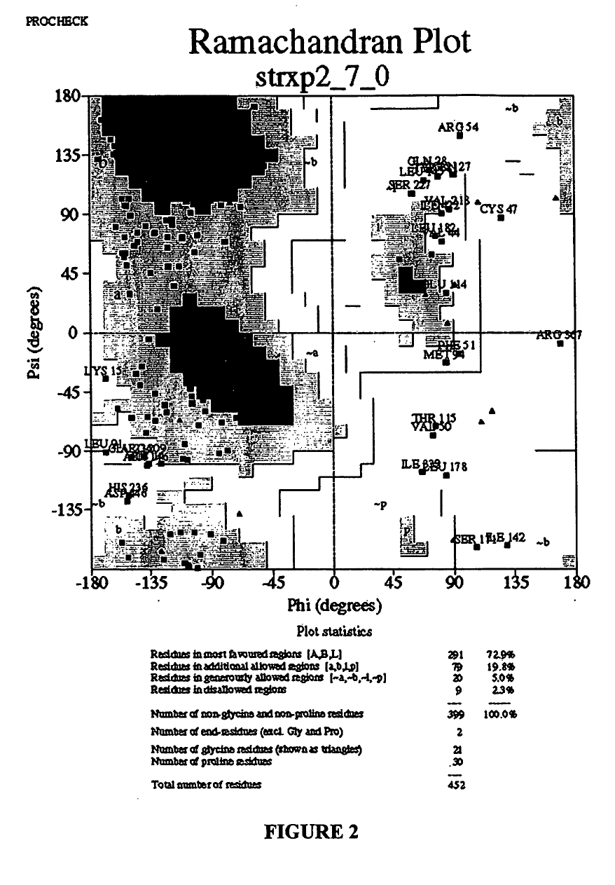

FIG. 2: Ramachandran plot of a lowest energy model of CYP3A4 produced by DYANA-XPLOR calculations from the six-template structural alignement.

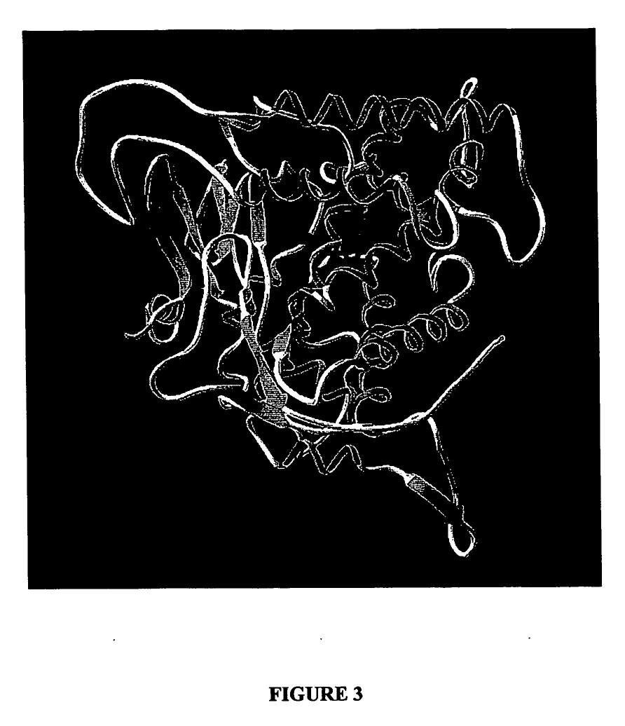

FIG. 3: view of one optimized CYP3A4 model. This figure can be replaced by the whole set of coordinates file of table 3 in the PDB format.

FIG. 4: final position of testosterone into the CYP3A4 and CYP3A7 active sites after soft-restrained dynamics docking. The active sites are characterized by six Substrates Recognition Sites (SRS, after Gotoh 1989, in bold) associated to fragments of secondary element structures (in italic).

Panel 4A In CYP3A4 active site, the docked testosterone molecule is oriented so that the A steroid cycle (carrying in position 3 a carbonyl function with an oxygen atom symbolized by a large ball ) is close to the heminic iron. This supports the propensity of CYP3A4 to metabolize testosterone in 6 β position as indicated by the black solid arrow.

Panel 4B In CYP3A7 active site, the docked testosterone molecule is oriented so that the D steroid cycle (carrying in position 17 a hydroxylic function with an oxygen atom symbolized by a large ball ) is close to the heminic iron. This supports the propensity of CYP3A7 to metabolize testosterone in 16 α position as indicated by the black solid arrow

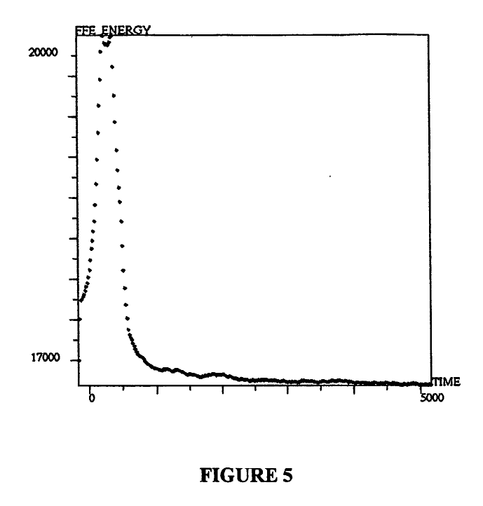

FIG. 5: Energy profile of the soft-restrained dynamics docking of testosterone into CYP3A4 model.

### EXAMPLE 1

### Determination of the 3D-structure of P450 3A4

Material

The coordinates of the six P450 crystal structures: P450cam (3cpp), P450terp (1cpt), P450BM-3 (2hpd), P450eryF (1oxa), P450 nor (1rom) and P450 2C5 (1dt6) were retrieved from the Brookhaven Protein data bank. The structural alignment and the conserved regions determination were realized using the GOK software (Jean et al. 1997) running on an Octane Silicon-Graphics workstation. Structures were built using the DYANA (Güntert et al. 1997), and X-PLOR softwares (Brünger 1992). Docking studies were performed with SYBYL 6.6 (Tripos Inc.) and TRIPOS force field. The structures were analyzed using Procheck-NMR (Laskowski et al. 1993) and visualized under SYBYL 6.6 (Tripos Inc.).

Common Structural Blocks (CSB) Determination.

The first key point of this homology modeling study is the identification of the structural elements (hereafter designed as CSBs for Common Structural Blocks) conserved among the family of cytochromes P450 of known 3D structures, and the localization of these elements in the target sequence. These two tasks are performed using the GOK software (Jean et al. 1997), and are well described in a forthcoming article (Minoletti et al., Proteins, Structure, Function and Genetics, 2002). In brief, the basic idea of CSB identification by GOK is to use an internal coordinate representation—(α,τ) in our case (another representation of φ, ψ and ω angles)—and to search for fragments in the six-template proteins having similar local trajectories in the internal coordinate space. GOK provides two adjustable parameters (the α-mesh and the α-margin) that define the tolerance on the comparison of the trajectories. These parameters were adjusted recursively to values ranging from 15 to 30° (α-mesh) and 1 to 3 (α-margin in mesh units). The evaluation of the quality of the match was measured using two multiple-way rmsd calculated in the cartesian coordinates space: mp-rms (the mean of all pairwise rms deviations) and s-rms (the mean of the deviations calculated with respect to a mean structure obtained from the average internal coordinates). For the different CSBs, mp-rms value ranged between 0.3 and 4.9 Å in average, and s-rms between 0.04 and 2.4 Å.

CYP3A4 Sequence Alignment and Evaluation of the Profile

The multiple sequence alignment derived from the CSB identification was then used to build a similarity profile. The profile is defined as a position-specific scoring table created from aligned gap-free segments such as CSBs (Jean et al. 1997). The alignment then consists in a search of the best match (as per the best score) between a CSB of sequences defined structurally (ie. independently of the nature of the aligned residues) and several other sequences that are well-aligned and exhibit a high sequence identity. In the P450 3A subfamily, many proteins exhibit high sequence identity. We extended our profile search program to take this information into account, i.e. to align the profile with a pre-defined multiple alignment of the cytochromes P450 3A subfamily members sequences (Gotoh 1992; Nelson et al. 1996). The similarity score was calculated using BLOSUM62 matrix (Henikoff and Henikoff 1992). The in-house tool SmartConsAlign (Atelier de Bio-informatique, Universitd Paris VI) described in Jean et al. 1997, allows to move the consensus matrix along the multiple sequence alignment of P450 3A family, and computes for each position a score of similarity. The best alignment found of CYP3A4 on CSBs is shown in FIG. 1.

Once the alignment is completed, the 3D model rebuilding process can incorporate the atom Cartesian coordinates of the template structures only for amino acids located in structurally conserved regions (i.e. the CSBs). The coordinates of any of the template structures can be used for determining the final template. In each CSB, amino acid positions have been renumbered according to the sequence of human P450 3A4. At a given position, when residues are identical between all the template structures and the target sequence, the 3D coordinates of the reference residues are purely assigned to the modeled (target) residue. When residues differ, only the coordinates of the backbone atoms are assigned (Cα), and sometimes Cβ when they exist. Side chains are rebuilt from libraries giving the most probable rotamers for each amino acid (see below). In some cases, it was possible to superimpose the positions of carbon atoms of lateral chains up to ranks γ and δ along the sidechain, thus explicitly defining a unique rotamer.

For amino acids located outside the CSBs (structurally variable zones that include generally loops), the rebuilding is more complex, and can be done only after rebuilding of structurally conserved zones. In the multiple structural alignment (FIG. 1), the regions separating the CSBs bring no structural information at all. Short loops are rebuilt entirely, since solutions of acceptable geometry for atoms are in limited number, i.e. the lowest energy drives the selection of the good geometry. For longer segments, various structures are provided by the constrained minimization runs, and a manual selection is operated.

Constraints Derivation and Rebuilding

A strategy inspired of the techniques commonly used to built structures from NMR data (Patard et al. 1996) is applied. The main idea is to express all available information issued from the comparison of the templates in term of geometrical constraints (distances and angles). Each constraint will be defined as an interval (for a given pair of atoms, this is the average of the six atom-atom distances found in the template structures±the standard deviation), similarly to the strategy developed by Havel and Snow (Havel and Snow 1991). However, the number of constraints corresponding to all atom-atom distances, for example, would be prohibitive for a protein of the size of the P450 (around 1,000,000 inter-residual distances if we consider 250 conserved residues and an average of four atoms per residues). Previous NMR studies (Patard et al. 1996) have shown that local constraints are sufficient to allow a correct reconstruction of a structure. This reduces drastically the number of constraints needed, and increases the flexibility of the model. In addition, similarly to what is done in protein structure determination by NMR, we can build a family of structures instead of a single model. This allows an easier analysis of the well or less well-predicted regions. This is also an advantage for the analysis of the side-chain positions, particularly in prevision of a substrate docking study. Finally, the loops are passively reconstructed with the rest of the structure. The only specific information we have introduced in variable regions was to guide all their residues to an allowed region of the Ramachandran diagram. Indeed, analysis of well-defined structures shows that nearly all residues, including those of the loops, should belong to an allowed region. The lower the proportion of residues found outside the allowed Ramachandran regions, the better the structure is. This criterion of quality has been applied to derive the model described herein.

Accordingly, we retained for model rebuilding all the distance and angle intervals corresponding to the following principles:
all distances for which the lower boundary was less than 8 Å. This cutoff is totally sufficient to ensure, at least, the formation of the local structure elements. Such a cutoff is relatively high and thus costly in terms of size of constraints file, but proved necessary to ensure good results for the P450s. This may be due to the fact that P450 enzymes are mostly formed of o-helices, the average distance between two helices being larger than between two adjacent β-strands. In addition, the percentage of residues located outside CSBs is rather high in the structural alignment of P450s, and a better convergence can be obtained only at the expense of a high number of rebuilding distance constraints.
all the distances involving at least one side-chain atom, to preserve the spatial arrangement between CSBs
finally, all the distances involving atoms of the heme group, to fix as much as possible the neighborhood of the iron atom.

The total number of distance constraints was, in these conditions, equal to 58506. Similarly, angular constraints were calculated in each building block. A CSB is indeed defined as a conserved trajectory in the φ,ψ coordinates space (or α,τ). Thus, dihedral angles φ and ψ of all residues located in CSBs can be defined as constraints, given by the average values of corresponding φ,ψ angles in the six templates±the standard deviation. To these backbone dihedral angles, can be added the side chains torsion angles χ₁, χ₂ whenever possible, as determined by the rotamer selection. The total number of dihedral angle constraints was, in these conditions, equal to 761.

Rotamer Selection

In proteins, the preferential orientation of the side chain (60°, −60°, 180°) depends on the local conformation of the residue, and thus on the nature of the secondary structure in which the residue is involved. According to the rotamer library built by Karplus and coil. (Dunbrack and Karplus 1993), to a given (φ, ψ) couple in the Ramachandran diagram can be associated a specific rotamer for each type of residue. These tables have been used to determine the most probable rotamer for each residue located in CSB, except when there are conserved atoms in the side chain that assign unambiguously a rotamer (χ₁, χ₂). The selected (χ₁, χ₂) couples were included in the above-mentioned set of angle 761 dihedral constraints.

Structure Calculation and Optimization

We used a procedure similar to structure calculation starting from NMR constraints. A first set of structures was calculated using the DYANA software (Guntert et al. 1997) and the 58506 distance and 761 angular constraints. Families of structures are generated. The energy of each structure is minimized with the procedure vtfmin in DYANA.

Due to the size and the amount of loops in the molecule, some structures presented topological defects and were discarded. The others were further optimized by using the X-PLOR software. A set of constraints was added at this stage in order to guide the loop residues to the nearest allowed region in the Ramachandran diagram. The topology and parameter files of CHARMM22 were used. The electrostatic term was turned off.

The DYANA software is unable to deal with disconnected objects. A new residue type was, thus, added to the standard amino acid library to take into account the the presence of the heme. This residue was obtained by combining the heme to a cysteine and was inserted at position 441 in the sequence of the protein (FIG. 1).

Description of the CYP3A4 Model

We rebuilt a model of the protein depleted of its first 50 residues (N-terminal domain). This segment is highly hydrophobic, and supposed to form the anchor of the protein in the membrane. There is no structural information about this putative transmembrane domain, and this segment was thus not incorporated into the modeling process, and in the final model. Such a “free” segment (with no constraints) would perturbate the convergence of computation or the stability of the whole rebuilt structure.

The quality of the various structures optimized under XPLOR was checked for the stereochemical quality (backbone and side chain conformation) by PROCHECK (Laskowski et al. 1993). The Ramachadran plot shows that our six-template approach generated converging models, possessing the same fold. The lowest energy models had 73% of their non-glycine and non-proline residues with φ, ψ conformation in the most favoured regions of the Ramachandran plot (core region), 20% in additional allowed regions, and 5% in the generously allowed regions. Only 2.3% (9 residues) had their φ, ψ conformation in disallowed regions (FIG. 2). The total number of residues in the model is 452; which 399 are non-glycine and non-proline residues, and number of residues in the native sequence is 502.

When compared to the CYP2C5 crystal structure, it can be noticed that the CYP3A4 model exhibits a good 3D similarity in the global fold than expected, since this structure counts only for one in the six-template approach. This proves that in this approach, there is no “averaging” effect, i.e. the mammalian structure had a decisive influence over the five bacterial (and fungus) templates. Our final fold of CYP3A4 is very consistent with a mammalian one, despite the fact that it has been rebuilt by using the structural information contained in non-mammalian cytochromes P450.

The active site is delimited by the six substrate recognition sites (SRS) that have been first identified and described by Gotoh (Gotoh 1992) from the unique structure available in the early 1990s (P450_{cam}), and that are today commonly accepted for depicting substrate recognition by various cytochromes P450 (especially from the family 2, but extended to other P450 families). These sites are associated with the active site and are located in the less conserved regions of the CYPs, thus possibly accounting for the various substrate specificity among P450s. When comparing our various optimized structures, it is found that SRS1 (100-125, includes helix B), SRS 2 (205-218, includes C-terminus of helix F), and SRS3 (237-249, includes N-terminus part of helix G) are located in less-defined regions, with significant variability in spatial position (flexibility). These regions correspond also to parts of the sequence that are less well-aligned. At the opposite, the SRS4 (295-320, central part of helix 1), SRS5 (363-380, C-term of helix K and β-sheet ,1-4) and SRS6 (470-490, β-sheets β4-1 and β4-2) are well-defined fragments of the structures. SRS4 and SRS5 segments in particular are correlated to regions in the sequence that are unequivocally aligned.

The only model structure of CYP3A4 that has been described in the literature and that we can handle for structural comparison, is that of Szklarz and Halpert, derived from a multiple-template approach (four-bacterial template) (Szklarz and Halpert 1997). Roughly, the same secondary structures are identified, but we found divergences in SRS location between their model and those derived from the present approach. SRS4 and SRS5 match well, but SRS2 is shifted (divergence in the position of helix F along the sequence), while SRS1 (helix B′), SRS3 (helix G) and SRS6 (sheet β4) are more notably displaced. The loops connecting the secondary structures of these SRS significantly disagree. These differences are likely to issue from a wrong alignment with the crystal P450 structures in the model of Szklarz and Halpert.

### EXAMPLE 2

### Determination of the 3D-structure of P450 3A7

The model rebuilding of CYP3A7 was performed according to the techniques described above in example 1 for CYP3A4, except that we used a restrained set of four-template structures, still including the mammalian CYP2C5, in order to test the robustness of the modeling approach. Below are pointed out only the differences in input data and the results relevant to CYP3A7.

Material

The coordinates of the four P450 crystal structures: P450BM-3 (2hpd), P450eryF (1oxa), P450 51-like from Mycobacterium tuberculosis (1e9x) and P450 2C5 (1dt6) were retrieved from the Brookhaven Protein data bank and used as initial template for GOK analysis.

Common Structural Blocks (CSB) Determination.

The GOK parameters were adjusted recursively to values ranging from 10 to 30° (α-mesh) and 1 to 3 (α-margin in mesh units). Occasionally, the α-mesh value was pushed up to 60° to refine some local structured loops (DE loop, HI loop) or short helices (such as J′). 27 CSBs have been identified. New CSBs were detected: the block 7* (between blocks 6 and 7A), the block 7B* (between 7B and 8) and the block 7C (between 7B* and 8). For the different CSBs, mp-rms value ranged between 0.12 and 4.57 Å in average.

The best alignment found of CYP3A7 on CSBs is shown in FIG. 1. On the 459 residues comprised in the model structure (the protein was rebuilt depleted of its first 44 residues from the N-terminal domain), 337 residues were found located in CSBs, i.e. 73% of residues belong to structurally conserved regions of the four-template set.

Constraints Derivation and Rebuilding

With a larger cutoff (12 Å), we obtained around 73000 distance constraints, and 900 dihedral constraints.

The residue covalently linked to the heme group is at position 442 in the sequence of the protein (FIG. 1).

Description of the CYP3A 7 Model

The four-template approach generated converging models, possessing the same fold. The PROCHECK analysis for structure quality assessment for the lowest energy models showed 74.4% of their non-glycine and non-proline residues with φ, ψ conformation in the most favoured regions of the Ramachandran plot (core region), 18.2% in additional allowed regions, and 4.7% in the generously allowed regions. 2.7% (11 residues) had their φ, ψ conformation in disallowed regions. The total number of residues in the model is 459; which 407 are non-glycine and non-proline residues, and number of residues in the native sequence is 503.

A closer inspection of the structure, and after the results of dynamics docking experiments (see below), revealed that several hydrogen bonds can hinder the main access to the active site. Thus, key residues that are likely to be involved in the recognition and admission of the substrate are Q79; F102; R105; R106; F108; F248; F304 and E374, and additionally C98 and C377 (FIG. 4B). More specifically, R105, R106, Q79 and E374 can establish mutual hydrogen bonds in one of the access channels, and are thus involved in the access of the substrate towards the active site.

### EXAMPLE 3

### Docking Strategy

Our aim in this example was to obtain the different positions of the known substrates of CYP3A in the active site, consistent with the oxidation sites and biochemical differences among the CYP3A isoforms. Considering the fact that the heme-binding site is deeply buried in the protein structure, and thus the selection and the pathway of the substrates within the enzyme structure are strongly dependent on the various possibilities of structure opening, we implemented a special approach more appropriate to flexible structures, hereafter referred as “restrained dynamics docking” or “soft-restrained dynamics docking”. This technique employs constrained molecular dynamics simulations, where the only constraints are heme-substrate distances. The successive steps are:

Conversion of the PDB XPLOR File in PDB for SYBYL File

The optimized structures with XPLOR (PDB format) are visualized with the SYBYL 6.6 software (Tripos Inc.), which implies a conversion of the file (atoms types correction) so as to make it compatible and exploitable in the constrained dynamics which will be performed with SYBYL.

Stabilization of the P450 3A4 Model Generated Under XPLOR

Then, we do agregate N^{o}1 (in the meaning of SYBYL) with all the NC_{α}CO atoms of the peptide backbone of the protein. The structure is relaxed with a dynamic of 10 ns at 100K followed by a minimization of 100 steps. Agregate N^{o}1 is then deleted.

We do agregate N^{o}2 constituted of the protein C_{α} only. The protein relaxation is reiterated with a dynamic of 10 ns at 100K and a minimization of 100 steps. Agregate N^{o}2 is then deleted.

The all protein is then relaxed with a first dynamic of 1 ns at 100K, followed by a dynamic of 1 ns at 200K and a dynamic of ions at 300K. We terminate with a minimization of 100 steps.

Restrained Dynamics Docking of the Substrate (Example: Testosterone)

We do agregate N^{o}3 constituted of all atoms outside a sphere of 20 Å around the C_{α} of residues constituting the heart of the B′ loop. We also add heminic iron to this aggregate.

The substrate is placed inside the protein, at around 30 Å from the heminic iron and next to SRS1 and SRS5 sites. The substrate is placed so that the contraints between the heminic iron and the substrate backbone go between SRS1, SRS5 and SRS3. Thus, for testosterone docking, we establish 4 distance contraints (limit below 3 Å, above 10 Å) between heminic iron and C3, C8, C10 and C13 carbons with a constraint of 2 kcal/Å on the entire structure so as to avoid to favour the approach of one part of the substrate more than the other.

We begin to perform a dynamic without contraints of the entire system at 20 K during 2 ns to stabilize the system, then we perform a dynamic under contraints at 20 K during 5 ns. We observe that the substrate worms between SRS1, SRS3 et SRS5 to reach a position at the vicinity of heminic iron. We terminate with a dynamic without contraints at 300 K to relax the system and we realize a minimization of 1000 steps.

Results

We found that the testosterone molecule is positioned at the vicinity of heminic iron in such way that the C6 of testosterone be at 4.9 Å of the iron, which is compatible with the hydroxylation of this compound to give 6β-hydroxy-testosterone (FIG. 4A).

Minimizations and dynamics with the SYBYL software are performed with the Tripos force field following the parameters: dielectric constant equal to 1 and distance-dependent, minimization method of POWELL, a minimum gradient of 0.05 kcal.mol⁻¹.Å⁻¹, electrostatics charges calculated according to the Gasteiger-Huckel method, and a NB cutoff of 8.0 Å (non-bond energies). The energetic diagram of dynamic docking of testosterone is shown in FIG. 5.

Interest of this Docking Strategy:

Most P450 isozymes recognize only one substrate (for specific catalysis in a metabolic pathway), or a very limited number of substrates, all chemically closely related. At the contrary, CYP 3A isozymes are known to recognize a large palette of substrates, and are also capable of multiple binding in the active site, up to three molecules in the vicinity of the heme, according to the model developed by Hosea et al. 2000. Multiple pharmacophoric behavior (Ekins et al. 2003), as well as allosteric or synergistic effects, characterize the members of this P450 subfamily.

The docking strategy described above can be easily extended to different binding and metabolism scenario.

For example, the docking of two or three testosterone molecules, or of two testosterone molecules and one alpha-naphtoflavone molecule (αNF) can be simulated in the following manner:
In a first step, a testosterone molecule is dynamically docked under constraints, and then released of its constraints to freely evolve in the active site and find a first bound equilibrium position.
In a next step, an external testosterone is presented, at the same entrance of the protein structure or in the vicinity of another access channel, and then dynamically docked under constraints. The system first evolves under constraints applied to the second molecule, and can be released for a subsequent free MD simulation of the two molecules bound in the active site. One can see the first bound molecule (testosterone or another substrate) to be re-oriented under the effect of the second docking, simulating a situation of cooperativity.
Similarly, the second molecule docked can be different from the first bound, e.g. a first testosterone bound to the active site followed by the docking of an αNF molecule, or the reverse situation.
One can combine of course the possibilities: for example, two molecules (identical or of different chemical nature) are docked following the two steps above, and then, after stabilization around an equilibrium position, a third molecule is introduced under constraints, and then released from its constraints to let the system evolving towards a favorable energetic conformational state. In this way, two αNF and one testosterone or one αNF and two testosterone can be docked.

Of course, not only substrates can be docked, but also inhibitors. The docking procedure above can help to measure the potential inhibitory power of a molecule, for example a compound comprising an imidazole group. A first step would include a standard constrained dynamic docking of the potential inhibitor, followed by a free MD simulation (constraints are released when the inhibitor is in the active site), or by a specifically-constrained MD simulation where the imidazole group is confined in the vicinity of the heminic iron by using an additional distance constraint Fe-imidazole. In a following step, a second substrate is dynamically docked under constraints from the exterior, and one can determine in what conditions the second molecule can chase the first one from its binding position. The strength of the additional constraint can be a measurement of the inhibitory potential.

Correspondingly, the exit pathway of the metabolites can be explored by simulating the exit of the molecule bound to the active site, using either free MD simulation (if the chemical nature of the transformed molecule allows an energetical instability), or using inverted constraints, i.e. soft distance constraints (between an external point and the bound molecule) that help to expel out the metabolite. Additionally, the best exit pathway can be deduced from the most favored energy profiles.

### REFERENCES

Aninat, C., Hayashi, Y., Andre, F., and Delaforge, M. 2001. Molecular requirements for inhibition of cytochrome p450 activities by roquefortine. Chem Res Toxicol. 14: 1259-1265.

Brünger, A. T. 1992. X-PLOR Version 3.1. A system for X-ray crystallography and NMR. version 3.1. Yale University Press, New Haven, Conn., USA.

Chothia, C., and Lesk, A. M. 1986. The relation between the divergence of sequence and structure in proteins. Embo J 5: 823-826.

Cupp-Vickery, J. R., and Poulos, T. L. 1995. Structure of cytochrome P450eryF involved in erythromycin biosynthesis. Nature Struct. Biology 2: 144-153.

Delaforge, M., André, F., Jaouen, M., Dolgos, H., Benech, H., Gomis, J. M., Noël, J. P., Cavelier, F., Verducci, J., Aubagnac, J. L., and Liebermann, B. 1997.

Metabolism of tentoxin by hepatic cytochrome P-450 3 A isozymes. Eur J Biochem. 250: 150-157.

Delaforge, M., Bouillé, G., Jaouen, M., Jankowski, C.K., Lamouroux, C., Bensoussan, C. 2001. Recognition and oxidative metabolism of cyclodipeptides by hepatic cytochrome P450. Peptides 22: 557-565.

Domanski, T. L., Liu, J., Harlow, G. R., and Halpert, J. R. 1998. Å nalysis of four residues within substrate recognition site 4 of human cytochrome P450 3 A4: role in steroid hydroxylase activity and alpha-naphthoflavone stimulation. Arch. Biochem. Biophys. 350: 223-232.)

Dunbrack, R. L. J., and Karplus, M. 1993. Backbone-dependent rotamer library for proteins- Application to side chain prediction. J. Mol. Biol. 230: 543-574.

Ekins, S., Stresser, D. M., and Williams, J. A. 2003. In vitro and pharrnacophore insights into CYP3A enzymes. Trends Pharmacol Sci. 24: 161-166.

Ferenczy, G., and Morris, G. 1989. The active site of cytochrome P450 nifedipine oxidation model building study. J. Mol. Graph 7: 206-211.

Gellner, K., Eiselt, R., Hustert, E., Arnold, H., Koch, I., Haberl, M., Deglmann, C. J., Burk, O., Buntefuss, D., Escher, S., Bishop, C., Koebe, H. G., Brinkmann, U., Klenk, H. P., Kleine, K., Meyer, U. A., and Wojnowski, L. 2001. Genomic organization of the human CYP3A locus: identification of a new inducible CYP3A gene. Pharmacogenetics. 11: 111-121.

Gotoh, O. 1992. Substrate Recognition Sites in Cytochrome-P450 Family-2 (CYP2) Proteins Inferred from Comparative Analyses of Amino Acid and Coding Nucleotide Sequences. Journal of Biological Chemistry 267: 83-90.

Guenguerich, F. P. 1995. Human cytochrome P450 enzymes. In “Cytochrome P450: structure, mechanism and biochemistry”, P. R. Ortiz de Montellano Ed., Plenum Press, pp. 537-574, New York.

Güntert, P., Mumenthaler, C., and Wüthrich, K. 1997. Torsion angle dynamics for NMR structure calculation with the new program DYANA. J. Mol. Biol. 273: 283-298.

Hasemann, C. A., Ravichandran, K. G., Peterson, J. A., and Deisenhofer, J. 1994. Crystal Structure and Refinement of Cytochrome P450(Terp) at 2.3 Å Resolution. J. Mol. Biol. 236: 1169-1185.

Havel, T. F., and Snow, M. E. 1991. A new method for building protein conformations from sequence alignments with homologues of known structure. J Mol Biol 217: 1-7.

Henikoff, S., and Henikoff, J. G. 1992. Amino acid substitution matrices from protein blocks. Proc Natll Acad Sci USA 89: 10915-10919.

Hilbert, M., Bohm, G., and Jaenicke, R. 1993. Structural relationships of homologous proteins as a fundamental principle in homology modeling. Proteins 17: 138-151.

Hosea, N. A., Miller, G. P., and Guengerich, F. P. 2000. Elucidation of distinct ligand binding sites for cytochrome P450 3 A4. Biochemistry 39: 5929-5939.

Inoue, E., Takahashi, Y., Imai, Y., Kamataki, T. Development of bacterial expression system with high yield of CYP3 A 7, a human fetus-specific form of cytochrome P450. Biochem Biophys Res Commun. 2000 Mar. 16;269(2):623-7.

Jean, P., Pothier, J., Dansette, P. M., Mansuy, D., and Viari, A. 1997. Automated multiple analysis of protein structures: application to homology modeling of cytochromes P450. Proteins 28: 388-404.

Karplus, M. and McCammon, J. A. 2002. Molecular dynamics simulations of biomolecules. Nat. Struct. Biol. 9: 646-652.

Koch, I., Weil, R., Wolbold, R., Brockmnoller, J., Hustert, E., Burk, O., Nuessler, A., Neuhaus, P., Eichelbaum, M., Zanger, U., Wojnowski, L. 2002. Interindividual variability and tissue-specificity in the expression of cytochrome P450 3 A mRNA. Drug Metab Dispos. 30: 1108-1114.

Laskowski, R. A., MacArthur, M., Moss, D. S., and Thorntorn, J. 1993. PROCHECK: a program to check the stereochemical quality of protein structures. J Appl. Crystallog. 26: 283-291.

Lewis, D. F. V. 2001. Guide to cytochrome P450 structure and function. Taylor & Francis, New York, pp. cm.

Lewis, D. F. V., Eddershaw, P. J., Goldfarb, P. S., and Tarbit, M. H. 1996. Molecular modelling of CYP3 A 4 from an alignment with CYP102: Identification of key interactions between putative active site residues and CYP3 A -specific chemicals. Xenobiotica 26: 1067-1086.

Loiseau, N. 2002. Conception d'analogues structuraux d'un cyclopeptide modele: étude du mode de reconnaissance moléculaire par trois systèmes enzymatiques membranaires. Université Paris XI, Orsay.

Nelson, D. R. 1999. Cytochrome P450 and the individuality of species. Arch Biochem Biophys 369: 1-10.

Nelson, D. R., Koymans, L., Kamataki, T., Stegeman, J. J., Feyereisen, R., Waxman, D. J., Waterman, M. R., Gotoh, O., Coon, M. J., Estabrook, R. W., et al. 1996. P450 superfamily: Update on new sequences, gene mapping, accession numbers and nomenclature. Pharmacogenetics 6: 1-42.

Park, S. Y., Shimizu, H., Adachi, S., Nakagawa, A., Tanaka, I., Nakahara, K., Shoun, H., Obayashi, E., Nakamura, H., Iizuka, T., et al. 1997. Crystal structure of nitric oxide reductase from denitrifying fungus Fusarium oxysporum. Nature Struct. Biology 4: 827-832.

Patard, L., Stoven, V., Gharib, B., Bontems, F., Lallemand, J. Y., and De Reggi, M. 1996. What function for human lithostathine? Structural investigations by three-dimensional structure modeling and high-resolution NMR spectroscopy. Protein Eng 9: 949-957.

Podust, L. M., Poulos, T. L., and Waterman, M. R. 2001. Crystal structure of cytochrome P450 14alpha -sterol demethylase (CYP51) from Mycobacterium tuberculosis in complex with azole inhibitors. Proc Natl Acad Sci USA 98: 3068-3073.

Poulos, T. L., Finzel, B. C., Gunsalus, I. C., Wagner, G. C., and Kraut, J. 1985. The 2.6 Å crystal structure of Pseudomonas putida cytochrome P450cam. J. Biol. Chem 260: 16122-16130.

Raag, R., and Poulos, T. L. 1989. Crystal structure of the carbon monoxide-substrat-cytochrome P-450cam ternary complex. Biochemistry 28: 7586-7592.

Ravichandran, K. G., Boddupalli, S. S., Hasemann, C. A., Peterson, J. A., and Deisenhofer, J. 1993. Crystal Structure of Hemoprotein Domain of P450BM-3, a Prototype for Microsomal P450's. Science 261: 731-736.

Sanchez, R., Pieper, U., Melo, F., Eswar, N., Marti-Renom, M. A., Madhusudhan, M. S., Mirkovic, N., and Sali, A. 2000. Protein structure modeling for structural genomics. Nat Struct Biol 7 Suppl: 986-990.

Schmiedlin-Ren, P., Edwards, D. J., Fitzsimmons, M. E., He, K., Lown, K. S., Woster, P. M., Rahman, A., Thummel, K. E., Fisher, J. M., Hollenberg, P. F., and Watkins, P. B. 1997. Mechanisms of enhanced oral availability of CYP3 A 4 substrates by grapefruit constituents. Decreased enterocyte CYP3 A 4 concentration and mechanism-based inactivation by furanocoumarins. Drug Metab Dispos. 25: 1228-1233.

Szklarz, G. D., and Halpert, J. R. 1997. Molecular modeling of cytochrome P450 3 A 4. J Comput Aid Molec Design 11: 265-272.

Westlind-Johnsson, A., Malmebo, S., Johansson, A., Otter, C., Andersson, T. B., Johansson, I., Edwards, R. J., Boobis, A. R., and Ingelman-Sundberg, M. 2003. Comparative analysis of CYP3A expression in human liver suggests only a minor role for CYP3 A 5 in drug metabolism. Drug Metab Dispos. 31: 755-761.

Williams, P. A., Cosme, J., Sridhar, V., Johnson, E. F., and McRee, D. E. 2000. Mammalian microsomal cytochrome P450 monooxygenase: structural adaptations for membrane binding and functional diversity. Molecular Cell 5: 121-131.

Williams, J. A., Ring, B. J., Cantrell, V. E., Jones, D. R., Eckstein, J., Ruterbories, K., Hamman, M. A., Hall, S. D., and Wrighton, S. A. 2002. Comparative metabolic capabilities of CYP3 A 4, CYP3 A 5, and CYP3 A 7. Drug Metab Dispos. 30: 883-891.

Wrighton, S. A., Schuetz, E. G., Thummel, K. E., Shen, D. D., Korzekwa, K. R., Watkins, P. B. 2000. The human CYP3A subfamily: practical considerations. Drug Metab Rev. 32: 339-361.

Yano, J. K., Koo, L. S., Schuller, D. J., Li, H., Ortiz de Montellano, P. R., and Poulos, T. L. 2000. Crystal structure of a thermophilic cytochrome P450 from the archaeon Sulfolobus solfataricus. J Biol Chem 275: 31086-31092.

### CROSS REFERENCE TO RELATED APPLICATIONS

This application is a national phase application of International Application Number PCT/IB2003/005134, filed Oct. 28, 2003, and claims the benefit of U.S. Provisional Application No. 60/421,569, filed on Oct. 28, 2002, the contents of both of which are incorporated herein by reference.

1. A method for designing a 3-dimentional (3-D) model of a protein, the 3-D representation of at least three family members has already been experimentally obtained, [said 3-D representation presenting similarities], comprising the steps of:
a. identification of common structural blocks (CSBs) among said members of said family,
b. alignment of the amino-acids primary sequence of said family members according to said structural similarities, represented by said CSBs, in order to obtain a first alignment,
c. alignment of said protein as compared on said first alignment, in order to obtain a second alignment, wherein:
i. alignment of said protein is performed in order to optimize the amino-acids alignment between said protein and said first alignment,
when one or more consensus amino-acid exists in said aligned CSBs in said first alignment, and in the amino-acid sequence of said protein, said consensus amino-acids are anchors of said second alignment,
ii. no insertion or deletion of amino-acids can be performed in the aligned CSBs, wherein insertion or deletions are possible in out-of-block regions, if better to align the primary amino-acids sequences,
d. definition of the 3-D structure of CSBS of said protein, according to the 3-D structure of the CSBs of said family members,
e. definition of the global constraints (distance and angular constraints) derived from the comparisons of the structural templates in CSBs, and definition of the local constraints (distance and angular constraints) for the atoms of residues that are not structurally determined after step d. (that are not in the CSBs),
f. selection of rotamers,
g. determination of a family of 3-D model structures of said protein, taking into account said 3-D structure of CSBs obtained in step d., said global and local constraints defined in step e., and said rotamers defined in step f.,
h. optimization of said family of 3-D models obtained in step g., by
i discarding structures that present topological defects, and
ii recalculating 3-D structures by taking electrostatic forces into account, and performing the method again from step c. downward, with modifications in the alignment between the primary sequence of said protein and said first alignment, when the obtained model structures do not satisfactorily account for known mutations having biological effects.

2. The method of claim 1, wherein said 3-D representation of family members has been obtained by crystallography or NMR.

3. The method of claim 1, wherein said alignment of said CSBs in step b. is performed by use of the GOK software.

4. The method of claim 1, wherein said alignment of said CSBs in step c. is performed by use of the GOK software.

5. The method of claim 1, wherein step d. is performed according to the following rules:
i. at a given position, when residues are identical between all the template structures and the target sequence, the 3D coordinates of the reference residues are purely assigned to the target residue,
ii. When residues differ, only the coordinates of the backbone atoms are assigned (Cα), and sometimes Cβ or Cγ when they exist.

6. The method of claim 1, wherein said definition of local constraints in step e. is performed by analysis of the allowed regions in Ramachandran diagram.

7. The method of claim 1, wherein global and local constraints are selected in step e. by the following rules:
i. all distances for which the lower boundary was less than 8 Å.
ii. all the distances involving at least one side-chain atom, to preserve the spatial arrangement between CSBs
iii. all the distances involving atoms of any active group such as an heme group, to fix as much as possible the neighborhood of said active group, such as an iron atom.

8. The method of claim 1, wherein angular constraints are selected in step e. by the following rule:
i. dihedral angles φ and ψ of all residues located in CSBs are defined as constraints, given by the average values of corresponding φ, ψ angles in said family members±the calculated standard deviation.

9. The method of claim 1, wherein said rotamers in step f. are selected from the couples according to the tables of Dunbrack and Karplus, where the choice of rotamers of a given amino acid is guided by the most favored zones in Ramachandran χ₁, χ₂ maps.

10. The method of claim 1, wherein said step g. is performed with the DYANA software.

11. The method of claim 1, wherein said optimization in step h. comprises the use of the X-Plor software, as described in A. T. Brunger, X-PLOR, version 3.1.

12. The method of claim 1, wherein said protein is a cytochrome P450 subfamily 3A comprising mammal and human cytochromes P450 3 A.

13. The method of claim 12, wherein said mammal cytochrome P450 3 A is is selected from the group comprising CYP3 A 6 (SEQ ID N^{o}14), CYP3 A 12 (SEQ ID N^{o}16), CYP3 A 29 (SEQ ID N^{o}17) and CYP3 A 13 (SEQ ID N^{o}18).

14. The method of claim 12, wherein said human cytochrome P450 subfamily 3A is selected from the group comprising CYP3 A 4 (SEQ ID N^{o}11), CYP3 A 7 (SEQ ID N^{o} 15), CYP3 A 5 (SEQ ID N^{o} 12) and CYP3 A 43 (SEQ ID N^{o} 13).

15. The method of claims 1 and 14, wherein said family members that are used for performing said first alignment for designing a 3-D model of CYP3 A 4 are chosen from Nor (SEQ ID N^{o} 1), Ery F (SEQ ID N^{o} 2), terp (SEQ ID N^{o} 3), Cam (SEQ ID N^{o} 4), BM3 (SEQ ID N^{o} 5) and 2C5 (SEQ ID N^{o} 6).

16. The method of claims 1 and 14, wherein said family members that are used for performing said first alignment for designing a 3-D model of CYP3 A 7 are chosen from Ery F (SEQ ID N^{o} 2), BM3 (SEQ ID N^{o} 5), CYP51 (SEQ ID N^{o} 8) and 2C5 (SEQ ID N^{o} 6).

17. A 3-D structure model of a protein, obtained by the method according to claim 1.

18. The model of claim 17, wherein said protein is a cytochrome P450 subfamily 3A comprising mammal and human cytochromes P450 3 A.

19. The model of claim 18, wherein said mammal cytochrome P450 3 A is selected from the group comprising CYP3 A 6 (SEQ ID N^{o}14), CYP3 A12 (SEQ ID N^{o}16), CYP3 A 29 (SEQ ID N^{o}17) and CYP3 A13 (SEQ ID N^{o}18).

20. The model of claim 18, wherein said human cytochrome P450 subfamily 3 A is selected from the group comprising CYP3 A 4 (SEQ ID N^{o}11), CYP3 A 7 (SEQ ID N^{o}15), CYP3 A 5 (SEQ ID N^{o}12) and CYP3 A 43 (SEQ ID N^{o}13).

21. The model of claim 20, wherein said protein is a cytochrome P450 3 A 4 or 3 A7.

22. The model of claim 21, wherein the residues C97; R104; F101; F107; F247; F303 and C376 are involved in the CYP 3 A 4 for the recognition and uptake of the substrate at the entry site, and its binding into the active site.

23. The model of claim 20, wherein the residues Q79; F102; R105; R106; F108; F248; F304 and E374 are involved in the CYP 3 A 7 for the recognition and uptake of the substrate at the entry site, and its binding into the active site.

24. The model of claim 22, having the 3-D atomic coordinates of Table 3.

25. The model of claim 23, having the 3-D atomic coordinates of Table 4.

26. A method for designing a protein, biological functions of which are altered, comprising:
a) obtaining a 3-D model of said protein by the method of claim 1,
b) analyzing said model of step a., and determining the amino-acids that are putatively involved in the biological functions of said protein,
c) changing said amino-acids by mutating the corresponding nucleotides on the nucleic acid sequence coding for said protein, in order to obtain a mutated protein having altered properties.

27. A computer-assisted method for performing restrained dynamics docking of a substrate on an enzyme, a 3-D structure of which is available, comprising the steps of
j. determining a force field, and independently simulating the presence of said enzyme in said force field,
k. minimizing the potential energy (Ep) linked to said force field of said 3-D structure, wherein the spatial position of some atoms of said enzyme is fixed, and wherein the other atoms are mobile, by allowing mobility of the mobile atoms, by
i. simulating an increase in temperature (in order to give kinetic energy),
ii. and minimizing the potential energy by re-specifying the temperature as 0 Kelvin (K)
l. optionally repeating step k in order to obtain other Ep minima, wherein said Ep minima are such that the structure of the protein remains folded,
m. minimizing Ep in said force field of said 3-D structure, wherein all the atoms of the protein are mobile, by
i. simulating an increase in temperature (in order to give kinetic energy), and
ii. minimizing the potential energy by re-specifying the temperature as 0 Kelvin (K)
n. simulating, at 0 K the presence of said substrate next to said enzyme,
o. optionally generating a molecular dynamics simulation on said substrate and enzyme (simulating an increase in temperature, in order to allow mobility of the atoms)
p. generating some constraints to said substrate, in order to impose that it has interaction with said enzyme,
q. generating a molecular dynamics simulation on said substrate and enzyme, with said constraints imposed in step p.,
r. optionally, generating a molecular dynamics simulation on said substrate and enzyme without said constraints of step p.

28. The method of claim 27, wherein said fixed atoms in step k. are the backbone atoms N—Cα-CO in the first minimization step and only C_{α} in subsequent minimization steps.

29. The method of claim 27, wherein said kinetic energy is simulated by temperature increase to about 100 K for about 5-20 ns.

30. The method of claim 27, wherein said force field in step j. comprises forces linked to
a. the distance between atoms,
b. the angles of valence,
c. the dihedral angles,
d. the deformation with regard to planar geometry,
e. the electrostatic field,
f. the Van der Waals forces,
g. hydrogen bonds.

31. The method of claim 27, wherein said constraints in step p. are attraction constraints to force said substrate in the active site, and wherein said constraints are not prejudiced to the exact spatial conformation of the substrate in the active site.

32. The method of claim 31, wherein said constraints are final distance constraints between some atoms of said substrate and some atoms of amino-acids present in said active site.

33. The method of claim 27, wherein step o. is performed with a simulated temperature of between about 15 and 50 K.

34. The method of claim 27, wherein step q. is performed with a simulated temperature of between about 15 and 50 K.

35. The method of claim 27, wherein step r. is performed with a simulated temperature of between about 200 and 350 K.

36. The method of claim 27, wherein said enzyme is a cytochrome P450 subfamily 3A comprising mammal and human cytochromes.

37. The method of claim 36, wherein said cytochrome is a cytochrome P450 3 A 4, and said structure is the structure obtained by the method of claim 15, in particular the model structure of claim 22.

38. The method of claim 36, wherein said substrate is a small organic compound which size can range for example from MW 288 (testosterone) to MW 1203 (cyclosporine A).

39. The method of claim 38, wherein said substrate is testosterone.

40. A computer-assisted method for performing restrained dynamics docking of at least two substrates on an enzyme, a 3-D structure of which is available, comprising the steps consisting of performing the steps depicted in claim 27 with a first substrate and repeating said steps with a second substrate when the first substrate reaches an unconstrained state after molecular dynamics simulation..

41. The method of claim 40, wherein the first and second substrates are the same molecule.

42. The method of claim 40, wherein the first and second substrates are different molecules.

43. The method of claim 41, wherein the first and second substrates display an allosteric effect.

44. The method of claim 41, wherein the first and second substrates display a synergistic effect.

45. The method of claims 41 and 42, wherein at least one of the substrates is an inhibitor or display an inhibitor-based mechanism.

46. The method of claims 41 and 42, wherein at least one of the substrates is an agonist.

47. The method of claim 40 comprising successively repeating the steps of claim 20 with a 3^{rd}, 4^{th} or 5^{th} substrate, some of them being the same or different molecules.

48. The method of claim 40, wherein said fixed atoms in step k. are the backbone atoms N—Cα-CO in the first minimization step and only C_{α} in subsequent minimization steps.

49. The method of claim 40, wherein said kinetic energy is simulated by temperature increase to about 100 K for about 5-20 ns.

50. The method of claim 40, wherein said force field in step j. comprises forces linked to:
a. the distance between atoms,
b. the angles of valence
c. the dihedral angles,
d. the deformation with regard to planar geometry,
e. the electrostatic field,
f. the Van der Waals forces
g. hydrogen bonds

51. The method of claim 40, wherein said constraints in step p. are attraction constraints to force said substrate in the active site, and wherein said constraints are not prejudiced to the exact spatial conformation of the substrate in the active site.

52. The method of claim 51, wherein said constraints are final distance constraints between some atoms of said substrate and some atoms of amino-acids present in said active site.

53. The method of claim 40, wherein step o. is performed with a simulated temperature of between about 15 and 50 K.

54. The method of claim 40, wherein step q. is performed with a simulated temperature of between about 15 and 50 K.

55. The method of claim 40, wherein step r. is performed with a simulated temperature of between about 200 and 350 K.

56. The method of claim 40, wherein said enzyme is a cytochrome P450 subfamily 3A comprising mammal and human cytochromes P450 3 A.

57. The method of claim 56, wherein said cytochrome is cytochrome P450 3 A 4, and said structure is the structure obtained by the method of claim 15, in particular the model structure of claim 22.

58. The method of claim 40, wherein said first and second substrates are small organic compounds which size can range from MW 288 (testosterone) to MW 1203 (cyclosporine A).

59. The method of claim 58, wherein said substrate is testosterone.

60. The use of the method according to claim 27 or 40 for screening, designing or identifying natural, unnatural substrates or substrate analogs, as well as inhibitors, activators or modulators of said enzyme.

61. The use of the method according to claim 40 or 47 for determining the effect of a first substrate on a second substrate.

62. The use according to claim 61 applied to pharmaceutical products.

63. The use of the method according to claim 40 or 47 for determining the effect of a first testosterone molecule on a second testosterone molecule.

64. The use of the method according to claim 40 or 47 for determining the effect of a first testosterone molecule on a second alpha-naphtoflavone molecule.

65. The use of the method according to claim 27 to 47 for determining the oxidative modification of the substrate according to the proximity to the heme of a part of the substrate.

66. The use of the method according to claim 27 to 39, or 40 to 47, for performing dynamic docking of the said metabolite, either in the absence or in the presence of the second substrate in the computed simulation.

67. The use of the method according to claims 27 to 39, or 40 to 47, to compare the energy of the bound metabolite relatively to the energy of its parent substrate bound, in order to determine if the exit of the given metabolite from the enzyme is favored or not.
